(19) United States
(12) Patent Application Publication
(10) Pub. No.: US 2011/0086834 A1
(43) Pub. Date: Apr. 14, 2011

(54) ALKYNYL ALCOHOLS AS KINASE INHIBITORS

(75) Inventors: Guoqing Chen, Palo Alto, CA (US); Timothy D. Cushing, Pacifica, CA (US); Paul Faulder, Cheshire (GB); Benjamin Fisher, San Mateo, CA (US); Xiao He, Foster City, CA (US); Kexue Li, Mountain View, CA (US); Zhihong Li, Millbrae, CA (US); Wen Liu, Foster City, CA (US); Lawrence R. Mcgee, Pacifica, CA (US); Vatee Pattaropong, Burlingame, CA (US); Jennifer L. Seganish, Scotch Plains, NJ (US); Youngshook Shin, Emeryville, CA (US); Zhulun Wang, Palo Alto, CA (US)

(73) Assignee: AMGEN INC., Thousand Oaks, CA (US)

(21) Appl. No.: 12/997,868

(22) PCT Filed: Jun. 26, 2009
(86) PCT No.: PCT/US09/03803

§ 371 Date: Dec. 13, 2010

Related U.S. Application Data
(60) Provisional application No. 61/133,249, filed on Jun. 26, 2008.

(51) Int. Cl. A61K 31/506; C07D 413/14; C07D 403/14; C07D 401/14; C07D 417/14; C07D 413/04; C07D 403/04; C07D 409/14; C07D 491/107; C07D 498/10; C07D 473/16; C07D 487/04; C07D 407/14; C07D 239/48; C07D 471/10; C07D 495/10; C07D 487/14; A61K 31/497; A61K 31/433; A61K 31/53; A61K 31/4439; A61K 31/427; A61K 31/52; A61K 31/5377; A61K 31/505; A61K 31/519; A61K 31/55; A61P 35/00; A61P 35/02; A61P 37/06; A61P 11/06; A61P 17/00; A61P 19/02; A61P 25/28; A61P 11/00; A61P 1/00; A61P 29/00

(52) U.S. Cl. 514/210.16; 544/324; 544/295; 548/128; 544/405; 544/209; 544/230; 544/71; 544/212; 546/269.7; 544/277; 548/181; 544/333; 544/122; 544/331; 544/323; 544/247; 540/521; 544/131; 544/328; 514/275; 514/255.05; 514/361; 514/245; 514/256; 514/230.5; 514/339; 514/365; 514/263.2; 514/235.2; 514/257; 514/212.06; 514/237.2

(57) ABSTRACT

Selected compounds are effective for prophylaxis and treatment of inflammation and inflammatory disorders, such as NIK-mediated disorders. The invention encompasses novel compounds, analogs, prodrugs and pharmaceutically acceptable salts thereof, pharmaceutical compositions and methods for prophylaxis and treatment of diseases and other maladies or conditions involving, inflammation and the like.

### FIELD OF THE INVENTION

This invention is in the field of pharmaceutical agents and specifically relates to compounds, compositions, uses and methods for treating inflammation and inflammatory disorders.

### BACKGROUND OF THE INVENTION

NIK is a member of the mitogen-activated protein kinase kinase kinase (MAP3K) family. It was originally identified as a serine/threonine protein kinase that interacts with TNF-receptor associated factor 2 (TRAF2) and stimulates the activation of the “classical” NF-κB pathway (Malinin, N. L., et al., (1997) Nature 385:540-4). NF-κB is a group of conserved eukaryotic transcription factors that regulate the expression of genes critical for both innate and adaptive immune responses (Hayden, M. S., and Ghosh, S. (2008) Cell 132, 344-362). The most prevalent or “classical” form of NF-κB is a heterodimer of p50 (also known as NF-κB1) and p65 (RelA), which normally retains in the cytoplasm as an inactive complex with the inhibitory proteins termed IκB. A wide variety of extracellular stimuli including the pro-inflammatory cytokine TNF can activate NF-κB by rapidly inducing the degradation of IκB (Chen, G., and D. V. Goeddel (2002) Science 296:1634-5). This allows NF-κB to translocate into the nucleus, where it activates the transcription of downstream genes. The degradation of IκB is dependent on the IκB-kinase (IKK) complex that phosphorylates IκB and tags it for proteasome-mediated degradation shortly after stimulation.

NIK has been suggested as an upstream kinase of IKK in the NF-κB pathway, as it binds and stimulates the enzyme activity of the IKK complex (see Regnier, C. H., et al. (1997) Cell 90:373-83; Woronicz, J. D., et al. (1997) Science 278:866-9; and Ling, L., Z. Cao, and D. V. Goeddel. (1998) Proc Natl Acad Sci USA 95:3792-7). However, gene-targeting experiments have clearly demonstrated that both IKK and NF-κB activation by various signals including TNF are normal in NIK-deficient cells (Yin, L., et al. (2001) Science 291:2162-5). To date, considerable evidence has accrued indicating that NIK is dispensable for the activation of the “classical” NF-κB pathway. Instead, it is indispensable for the activation of a second major form of NF-κB, consisting of a heterodimer of p52 (NF-κB2) and Rel B (see Hayden, M. S., and Ghosh, S. (2008) Cell 132, 344-362; Pomerantz, J. L., and D. Baltimore. (2002) Mol Cell 10:693-5; and Dixit, V., and T. W. Mak. (2002) Cell 111:615-9). In most types of cells, only a small amount of p52 is present relative to its precursor p 100. Like IκB, unprocessed p100 can function as a cytoplasmic inhibitor for NF-κB (Hayden, M. S., and Ghosh, S. (2008) Cell 132, 344-362). Overexpression of NIK promotes the processing of NF-κB2 precursor p100 to its active form p52 (Xiao, G., et al. (2001) Mol Cell 7:401-9), which together with Rel B binds DNA and activates the transcription of targeted genes. Moreover, p100 processing or NF-κB2 activation is defective in the absence of functional NIK (see Pomerantz, J. L., and D. Baltimore. (2002) Mol Cell 10:693-5; and Dixit, V., and T. W. Mak. (2002) Cell 111:615-9).

### NIK Controls B Cell Maturation and Secondary Lymphoid Organogenesis

NIK^{-/-} mice are grossly normal but show abnormal development of B cells and secondary lymphoid organs (Yin, L., et al. (2001) Science 291:2162-5). NIK^{-/-} mice lack all peripheral lymph nodes (LN) and fail to form Peyer's patches. The spleen and thymus also exhibit disrupted architecture. The number of mature B cells in NIK^{-/-} mice is reduced ˜60% comparing to that in wild type (WT) mice. In contrast, the numbers of other types of immune cells, including T cells and macrophages, are essentially normal. NIK^{-/-} mice have undetectable levels of serum immunoglobulin A (IgA) and greatly reduced (>60 fold) levels of IgG_{2b}. NIK^{-/-} mice are severely compromised in their capacity to mount antibody responses to foreign antigen challenge. In spite of these defects, NF-κB activation in response to TNF and many other stimuli are normal in the absence of NIK.

NIK^{-/-} mice share many deficits with alymphoplasia (aly/aly) mice (Miyawaki, S., et al. (1994) Eur J Immunol 24:429-34), a natural mutant strain that carries a single point mutation near the carboxyl-terminus of NIK (Shinkura, R., et al. (1999) Nat Genet. 22:74-7; Fagarasan, S., et al. (2000) J Exp Med 191:1477-86; and Yamada, T., et al. (2000) J Immunol 165:804-12). aly/aly mice are characterized by the systemic absence of LN and Peyer's patches, and the disorganized spleen and thymus structures. In addition, they have a decreased level of IgM and extremely low levels of IgG and IgA. Mature B cell numbers are markedly reduced in aly/aly mice, which are deficient in both humoral and cell-mediated immune responses. However, the mutant mice are still sensitive to lipopolysaccharide (LPS)-induced endotoxic shock. Up-regulation of the NF-κB-mediated genes in response to TNF and other pro-inflammatory cytokines is also intact in aly/aly mice.

### NIK is Required for BAFF-R Signaling to B Cell Maturation

BAFF (also known as BLyS, TALL-1, THANK, and zTNF4) is a member of the TNF-family and primarily produced by macrophages, monocytes, and dendritic cells (Mackay, F., et al. (2003) Annu Rev Immunol 21:231-64; Locksley, R. M., et al. (2001) Cell 104:487-501; Fagarasan, S., and T. Honjo. (2000) Science 290:89-92; and Waldschmidt, T. J., and R. J. Noelle. (2001) Science 293:2012-3). The binding of BAFF to its cognate receptor BAFF-R, which is almost exclusively expressed on B cells, stimulates B cell growth and function (Moore, P. A., et al. (1999) Science 285:260-3; Schneider, P., et al. (1999) J Exp Med 189:1747-56; Thompson, J. S., et al. (2001) Science 293:2108-11; and Yan, M., et al. (2002) Curr Biol 12:409-13). Evidence from extensive genetic and biochemical studies have made it clear that BAFF signals its activity through BAFF-R, which in turn initiates a NIK-dependent process, ultimately leading to the activation of the NF-κB2 pathway.

The production of p52 appears to correlate with the process of B cell maturation as its levels progressively increase in the mature and terminally differentiated B cells (Liou, H. C., et al. (1994) Mol Cell Biol 14:5349-59). The treatment of B cells with BAFF readily induces the processing of p100 to p52 (Claudio, E., et al. (2002) Nat Immunol 3:958-65). In contrast, administration of BAFF-neutralizing soluble receptor proteins to mice inhibits p100 processing in vivo and lowers the basal p52 levels in B cells (Kayagaki, N., et al. (2002) Immunity 17:515-24). Moreover the enzyme activity of NIK is essential for BAFF-induced p100 processing to p52 (Xiao, G., et al. (2001) Mol Cell 7:401-9; and Senftleben, U., et al. (2001) Science 293:1495-9). Therefore, a NIK-specific small molecule inhibitor will provide a powerful tool for blocking BAFF/BAFF-R signaling activity.

### NIK is Required for LTβ-R Signaling to Secondary Lymphoid Organogenesis

Lymphotoxin β receptor (LTβ-R) signaling represents a second pathway that signals its activity by promoting NIK-dependent p100 processing (Pomerantz, J. L., and D. Baltimore. (2002) Mol Cell 10:693-5; Dixit, V., and T. W. Mak. (2002) Cell 111:615-9; and Locksley, R. M., et al. (2001) Cell 104:487-501). The binding of agonistic LTβ-R antibody or its natural ligand, which is a heterotrimer of LTa and LT13 (LTα/β2), induces the processing of p100 to p52 (Dejardin, E., et al. (2002) Immunity 17:525-35; Muller, J. R., and U. Siebenlist. (2003). J Biol Chem 278:12006-12; Yilmaz, Z. B., et al. (2003) Embo J 22:121-30; and Fu, Y. X., and D. D. Chaplin. (1999) Annu Rev Immunol 17:399-433). LTβ-R is expressed predominantly on the non-lymphoid cells such as stromal cells, while the expression of its ligand is restricted to the activated lymphocytes (Crowe, P. D., et al. (1994) Science 264:707-10; and Browning, J. L., et al. (1993) Cell 72:847-56). Administration of LTα in vivo or Tg mice overexpressing LTα leads to the ectopic formation of lymph node-like tissues (Rennert, P. D., et al. (1998) Immunity 9:71-9; and Luther, S. A., et al. (2000) Immunity 12:471-81). The blockade of LTβ-R signaling by LT-neutralizing soluble receptor proteins results in the loss of secondary lymphoid organs (Schrama, D., et al. (2001) Immunity 14:111-21). Mice with targeted disruption of genes encoding LTβ-R or its ligand do not develop secondary lymphoid organs (Rennert, P. D., et al. (1996) J Exp Med 184:1999-2006; De Togni, P., et al. (1994) Science 264:703-7; and Futterer, A., et al. (1998) Immunity 9:59-70), a phenotype that is qualitatively similar to that of NIK^{-/-} mice. A stromal defect caused by impaired NIK-dependent LTβ-R signaling may account for abnormal development of secondary lymphoid organs in NIK^{-/-} mice.

### NIK is Required for RANK Signaling to Osteoclastogenesis

NIK has also been known to play a critical role in the signaling pathways elicited by several other TNF-family cytokines, including CD27L, CD40L, TWEAK and RANKL (receptor activator of NF-kappaB ligand) (Ramakrishnan, P., et al. (2004) Immunity 21, 477-489). Mice lacking functional NIK have impaired RANKL-stimulated formation of osteoclasts (Novack, D. V., et al. (2003) J Exp Med 198, 771-781), which are multinucleated cells from bone marrow responsible for removing the mineralized matrix of bone tissues. In the absence of NIK, p100 expression is increased by RANKL, but its conversion to p52 is blocked, leading to cytosolic accumulation of p100. High levels of unprocessed p100 in osteoclast precursors from NIK^{-/-} mice or a nonprocessable form of the protein in wild-type cells impair RANKL-mediated osteoclastogenesis. NIK is also required for osteoclastogenesis in response to pathologic stimuli. Tumor-induced osteolysis in NIK^{-/-} mice is completely blocked while growth of tumor cells in the bone marrow is similar to WT controls (Vaira, S., et al. (2008) Proc Natl Acad Sci USA 105, 3897-3902).

### Excess NIK-Mediated NF-κB2 Signaling Leads to Autoimmunity

Overproduction of BAFF is known to associate with the pathogenesis of various autoimmune conditions in humans and animals (Kalled, S. L. (2002) Curr Opin Investig Drugs 3:1005-10). Transgenic mice overexpressing BAFF exhibit vastly increased numbers of B cells with severely enlarged secondary lymphoid organs and abnormally elevated levels of serum immunoglobulins. They also develop a systemic lupus erythematosus (SLE)-like autoimmune phenotype (Mackay, F., et al. (1999) J Exp Med 190:1697-710; Gross, J. A., et al. (2000) Nature 404:995-9; and Khare, S. D., et al. (2000) Proc Natl Acad Sci USA 97:3370-5). As BAFF Tg mice age, they develop a secondary pathology reminiscent of Sjögren's syndrome (SS), in which end organ damages are in the inflamed salivary and lacrimal glands (Groom, J., et al. (2002) J Clin Invest 109:59-68). In humans, BAFF levels correlate with the disease severity of autoimmune syndromes, including SLE, SS and rheumatoid arthritis (RA) (Groom, J., et al. (2002) J Clin Invest 109:59-68; Zhang, J., et al. (2001) J Immunol 166:6-10; and Cheema, G. S., et al. (2001) Arthritis Rheum 44:1313-9).

The formation of secondary lymphoid organ-like tissues is a prototypic feature of many chronic inflammatory and autoimmune conditions, including RA and inflammatory bowel diseases (IBD) (Fu, Y. X., and D. D. Chaplin. (1999) Annu Rev Immunol 17:399-433). Administration of soluble LT-neutralizing receptor proteins reverses the formation of lymphoid organ-like structures and prevents the development of colitis, arthritis, and insulin-dependent diabetes mellitus in mouse models (Shao, H., et al. (2003) Eur J Immunol 33:1736-43; Ettinger, R., et al. (2001) J Exp Med 193:1333-40; and Wu, Q., et al. (2001) J Exp Med 193:1327-32). NIK^{-/-} mice were completely resistant to antigen-induced arthritis (AIA) and to a genetic, spontaneous form of arthritis (Aya, K., et al. (2005) J Clin Invest 115, 1848-1854). These mice also showed significantly less osteoclastogenesis and bone erosion in the serum transfer arthritis model. NIK is important in both the immune and bone-destructive components of inflammatory arthritis, indicating that NIK-specific inhibitors have the potential for the treatment of these chronic inflammatory diseases.

### Excess NIK-Mediated NF-κB2 Activities Lead to Malignancy

Intense B lymphocyte activities caused by excess NIK-mediated NF-κB2 activities have been implicated in various types of cancer, in particular lymphoma, leukemia and multiple myeloma (Mackay, F., et al. (2003) Annu Rev Immunol 21:231-64; Saitoh, Y., et al. (2008) Blood 111, 5118-5129; Annunziata, C. M., et al. (2007) Cancer Cell 12, 115-130; and Keats, J J., et al. (2007) Cancer Cell 12, 131-144). For example, the BAFF gene is located on a human chromosome locus frequently involved in chromosomal translocations in patients with Burkitt lymphoma-leukemia and elevated levels of BAFF are detected in sera from non-Hodgkin's lymphoma (NHL) patients. Overexpression of BAFF in mice causes the development of a submaxillary gland tumor that is composed essentially of hyperplastic B cells (Groom, J., et al. (2002) J Clin Invest 109:59-68). The NF-κB2 gene was cloned from a B cell lymphoma-associated chromosomal translocation (Neri, A., et al. (1991) Cell 67:1075-87). The translocation results in the production of a carboxyl-terminal truncated protein that is constitutively active and tumorigenic (Clana, P., et al. (1997) Oncogene 14:1805-10; and Fracchiolla, N. S., et al. (1993) Oncogene 8:2839-45). NF-κB2 rearrangements are present in ˜2% of human lymphoid tumors. Overexpression of NIK also contributes to the tumorigenesis of adult T-cell leukemia and Hodgkin Reed-Sternberg cells (Saitoh, Y., et al. (2008) Blood 111, 5118-5129). NIK is also involved in the pathogenesis of multiple myeloma (MM) (Annunziata, C. M., et al. (2007) Cancer Cell 12, 115-130; and Keats, J. J., et al. (2007) Cancer Cell 12, 131-144). Two independent studies demonstrate that MM derived cell lines or clinical samples frequently have elevated expression of NIK due to genetic or epigenetic alterations, leading to the constitutive activation of the NF-kB2 pathway.

### DESCRIPTION OF THE INVENTION

A class of compounds useful in treating inflammation is defined by formula I

A compound of formula I

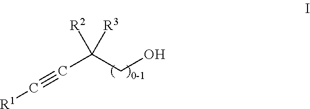

enantiomers, diastereomers and salts thereof wherein
R¹ is selected from

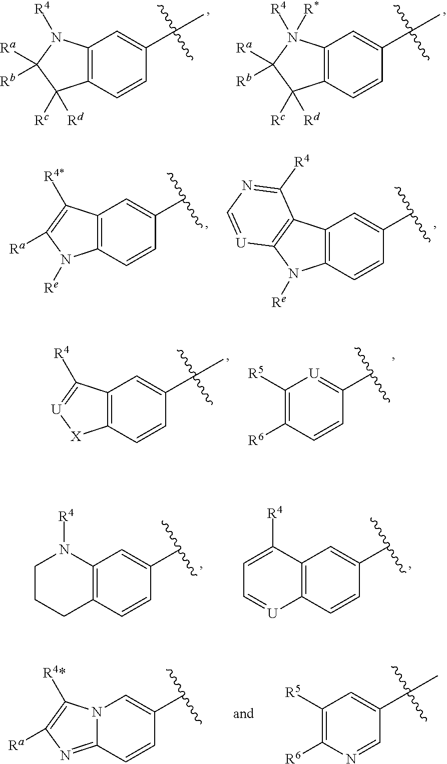

any of which may be optionally substituted with one or more R^{x} groups as allowed by valance;

provided that when R¹ is

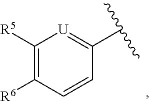

where U is CH and R⁶ is H, then R⁵ is other than

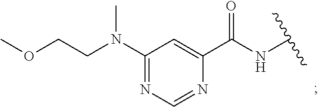

R² is alkyl or haloalkyl;
R³ is alkyl, cycloalkyl, haloalkyl, —C(═O)R⁷, —C(═O)OR⁷, —C(═O)NR⁸R⁹, aryl or heteroaryl wherein either of said aryl or heteroaryl may be optionally substituted with one or more R^{x} as allowed by valance;
or R² and R³ together with the carbon atom to which they are attached may combine to form

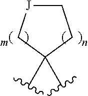

which may be optionally substituted with one or more R^{x} groups as allowed by valance;
R⁴ and R⁴* are independently
   i) pyridyl, pyrimidyl, pyrazinyl, triazinyl, purinyl, pyrrolopyrimidyl, triazolopyrimidyl, furopyrimidyl, thienopyrimidyl, oxazolopyrimidyl, or thiazolopyrimidyl, each of which is substituted with at least one R¹⁰ group, and any of which may be optionally substituted with one or more R^{x} groups as allowed by valance; or
   ii) oxazolyl, isoxazolyl, oxadiazolyl, thiazolyl, isothiazolyl, thiadiazolyl, or —C(═O)R⁷* any of which may be optionally substituted with one or more R^{x} groups as allowed by valance;
      provided R⁴ is other than —C(═O)R⁷* when R¹ is

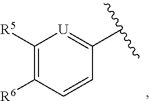

where U is CH and R⁶ is H, and R⁵ is either —(CH₂)ₖ—N(R⁸)(R⁴) or —(CH₂)ₖ—R⁴;
R⁵ is —(CH₂)ₖ—R⁴, —(CH₂)ₖ—N(R⁸)(R⁴), —(CH₂)ₖ—OR⁴, —(CH₂)ₖ—C(═O)R⁴; —(CH₂)ₖ—C(═O)OR⁴, —(CH₂)ₖ—C(═O)N(R⁸)(R⁴) or —(CH₂)ₖ—NR⁸—(C═O)R⁴;
R⁶ is H, halo, alkyl, —(CH₂)ₖ—OR¹¹, —(CH₂)ₖ—N(R¹²)(R¹³), —(CH₂)ₖ—C(═O)R¹¹, —(CH₂)ₖ—C(═O)OR¹¹;
R⁷, R⁷* and R⁷⁺ are each independently
   (i) H, or
   (ii) alkyl, alkenyl, alkynyl, cycloalkyl, heterocyclyl, aryl, heteroaryl, cycloalkylalkyl, heterocyclylalkyl, arylalkyl, heteroarylalkyl, alkoxyalkyl, hydroxyalkyl or NR⁸R⁹-alkyl any of which may be optionally substituted with one or more R^{x} groups as allowed by valance;
R⁸, R⁹, R⁸⁺ and R⁹⁺ are each independently
   (i) H;
   (ii) alkyl, alkenyl, alkynyl, cycloalkyl, heterocyclyl, aryl, heteroaryl, cycloalkylalkyl, heterocyclylalkyl, arylalkyl, heteroarylalky, alkoxyalkyl, hydroxyalkyl or (NR¹²R¹³)-alkyl, any of which may be optionally substituted with one or more R^{x} groups as allowed by valance;
   (iii) or R⁸ and R⁹ together with the nitrogen atom to which they are attached may combine to form a heterocyclyl ring optionally substituted with one or more R^{x} groups as allowed by valance;
   (iv) or R⁸⁺ and R⁹⁺ together with the nitrogen atom to which they are attached may combine to form a heterocyclyl ring optionally substituted with one or more R^{x} groups as allowed by valance;
R¹⁰ is H, —NR¹⁴R¹⁵, or —C(═O)NR¹⁴R¹⁵;
R¹¹ is
   (i) H, or
   (ii) alkyl, alkenyl, alkynyl, cycloalkyl, heterocyclyl, aryl, heteroaryl, cycloalkylalkyl, heterocyclylalkyl, arylalkyl or heteroarylalkyl any of which may be optionally substituted with one or more R^{x} groups as allowed by valance;
R¹² and R¹³ are each independently
   (i) H;
   (ii) alkyl, alkenyl, alkynyl, cycloalkyl, heterocyclyl, aryl, heteroaryl, cycloalkylalkyl, heterocyclylalkyl, arylalkyl, heteroarylalkyl, alkoxyalkyl, hydroxyalkyl or (NR⁸R⁹)-alkyl, any of which may be optionally substituted with one or more R^{x} groups as allowed by valance;
   (iii) or R¹² and R¹³ together with the nitrogen atom to which they are attached may combine to form a heterocyclyl ring optionally substituted with one or more R^{x} groups as allowed by valance;
R¹⁴ and R¹⁵ are each independently
   (i) H;
   (ii) alkyl, alkenyl, alkynyl, cycloalkyl, heterocyclyl, aryl, heteroaryl, cycloalkylalkyl, heterocyclylalkyl, arylalkyl, heteroarylalkyl, alkoxyalkyl, hydroxyalkyl or (NR¹²R¹³)-alkyl, any of which may be optionally substituted with one or more R^{x} groups as allowed by valance;
   (iii) or R¹⁴ and R¹⁵ together with the nitrogen atom to which they are attached may combine to form a heterocyclyl ring optionally substituted with one or more R^{x} groups as allowed by valance;
R^{a}, R^{b}, R^{d} and R* are each independently H or R^{x}
   or R^{a} and R^{b} together with the carbon atom to which they are attached may combine to form a carbonyl group;
   or R^{b} together with either R^{c} or R* may combine to form a bond;
   or R^{a} and R^{e}, together with the atoms to which they are respectively attached, may combine to form

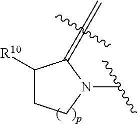

which may be optionally substituted with one or more R^{x} groups as allowed by valance;
or R^{a} and R⁴*, together with the atoms to which they are respectively attached, may combine to form

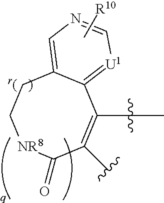

which may be optionally substituted with one or more R^{x} groups as allowed by valance;
or R^{c} and R^{d} together with the carbon atom to which they are each attached may combine to form

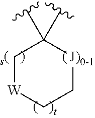

which may be optionally substituted with one or more R^{x} groups as allowed by valance;
R^{e} is H, R⁴, or R^{x};
R^{x} is an optional substituent independently selected at each occurrence from halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, -(alkylene)_{q}-OR⁷⁺, -(alkylene)_{q}-S(O)ᵥR⁷⁺, -(alkylene)_{q}-NR⁸⁺R⁹⁺, -(alkylene)_{q}-C(═O)R⁷⁺, -(alkylene)_{q}-C(═S)R⁷⁺, -(alkylene)_{q}-C(═O)OR⁷⁺, -(alkylene)_{q}-OC(═O)R⁷⁺, -(alkylene)_{q}-C(═S)OR⁷⁺, -(alkylene)_{q}-C(═O)NR⁸⁺R⁹⁺, -(alkylene)_{q}-C(═S)NR⁸⁺R⁹⁺, -(alkylene)_{q}-N(R¹⁵)C(═O)NR⁸⁺R⁹⁺, -(alkylene)_{q}-N(R¹⁵)C(═S)NR⁸⁺R⁹⁺, -(alkylene)_{q}-N(R¹⁵)C(═O)R⁷⁺, -(alkylene)_{q}-N(R¹⁵)C(═S)R⁷⁺, -(alkylene)_{q}-OC(═O)NR⁸⁺R⁹⁺, -(alkylene)_{q}-OC(═S)NR⁸⁺R⁹⁺, -(alkylene)_{q}-SO₂NR⁸⁺R⁹⁺, -(alkylene)_{q}-N(R¹⁵)SO₂R⁷⁺, -(alkylene)_{q}-N(R¹⁵)SO₂NR⁸⁺R⁹⁺, -(alkylene)_{q}-N(R¹⁵)C(═O)OR⁷⁺, -(alkylene)_{q}-N(R¹⁵)C(═S)OR⁷⁺, or -(alkylene)_{q}-N(R¹⁵)SO₂R⁷⁺;
   wherein alkylene, alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more -(alkylene)_{q}-CN, -(alkylene)_{q}-OR⁷⁺, -(alkylene)_{q}-S(O)ₙR⁷⁺, -(alkylene)_{q}-NR⁸⁺R⁹⁺, -(alkylene)_{q}-C(═O)R⁷⁺, -(alkylene)_{q}-C(═S)R⁷⁺, -(alkylene)_{q}-C(═O)OR⁷⁺, -(alkylene)_{q}-OC(═O)R⁷⁺, -(alkylene)_{q}-C(═S)OR⁷⁺, -(alkylene)_{q}-C(═O)NR⁸⁺R⁹⁺, -(alkylene)_{q}-C(═S)NR⁸⁺R⁹⁺, -(alkylene)_{q}-N(R¹⁵)C(═O)NR⁸⁺R⁹⁺, -(alkylene)_{q}-N(R¹⁵)C(═S)NR⁸⁺R⁹⁺, -(alkylene)_{q}-N(R¹⁵)C(═O)R⁷⁺, -(alkylene)_{q}-N(R¹⁵)C(═S)R⁷⁺, -(alkylene)_{q}-OC(═O)NR⁸⁺R⁹⁺, -(alkylene)_{q}-OC(═S)NR⁸⁺R⁹⁺, -(alkylene)_{q}-SO₂NR⁸⁺R⁹⁺, -(alkylene)_{q}-N(R¹⁵)SO₂R⁷⁺, -(alkylene)_{q}-N(R¹⁵)SO₂NR⁸⁺R⁹⁺, -(alkylene)_{q}-N(R¹⁵)C(═O)OR⁷⁺, -(alkylene)_{q}-N(R¹⁵)C(═S)OR⁷⁺, or -(alkylene)_{q}-N(R¹⁵)SO₂R⁷⁺;
J and W are independently —CH₂—, —N(R⁸)—, —O—, or —S(═O)ᵥ—,
X is —O— or —S(═O)ᵥ—,
U and U¹ are independently CH or N
k at each occurrence is independently 0, 1, 2 or 3;
m is 1, 2 or 3
n is 0, 1 or 2
p is 1, 2 or 3
q at each occurrence is independently 0 or 1
r is 0, 1, 2 or 3;
s is 1, 2 or 3
t is 0, 1 or 2
v at each occurrence is independently 0, 1 or 2

Preferred compounds of the current invention include compounds wherein R¹ is selected from

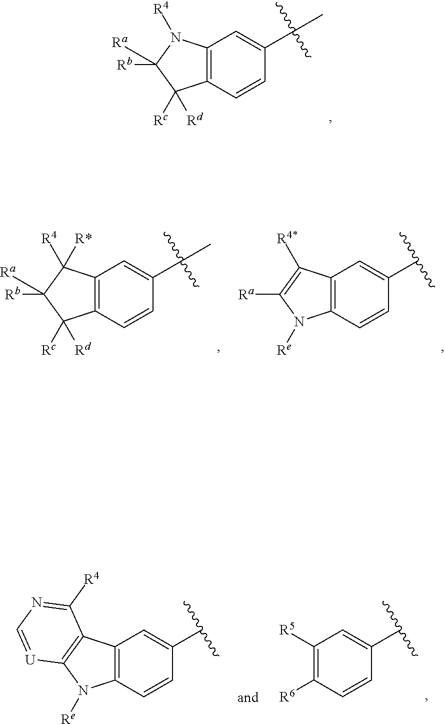

More preferred compounds of the current invention include compounds wherein R¹ is selected from

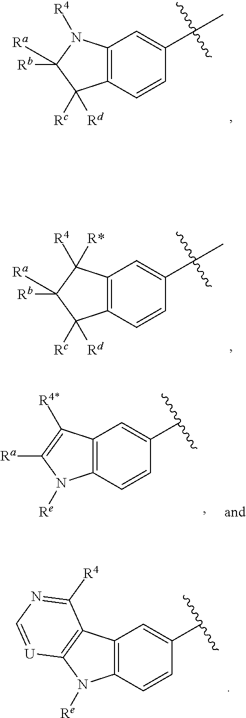

Preferred compounds of the present invention include compounds wherein
R² is alkyl; and
R³ is pyrrolyl, furanyl, thienyl, imidazolyl, pyrazolyl, oxazolyl, isoxazolyl, thiazolyl, isothiazolyl, phenyl, pyridyl, pyrimidyl, pyrazinyl, or oxadiazolyl any of which may be optionally substituted with one or more R^{x} as allowed by valance.
or R² and R³ together with the carbon atom to which they are attached may combine to form

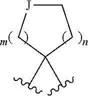

which may be optionally substituted with one or more R^{x} groups as allowed by valance; (especially preferred compounds include those where R² is alkyl; and
R³ is pyrrolyl, furanyl, thienyl, imidazolyl, pyrazolyl, oxazolyl, isoxazolyl, thiazolyl, isothiazolyl, phenyl, pyridyl, pyrimidyl, pyrazinyl, or oxadiazolyl any of which may be optionally substituted with one or more R^{x} as allowed by valance;
or R² and R³ together with the carbon atom to which they are attached may combine to form

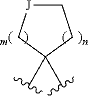

which may be optionally substituted with one or more R^{x} groups as allowed by valance).

Preferred compounds of the present invention include those wherein R², R³ and the carbon atom to which they are attached are selected from

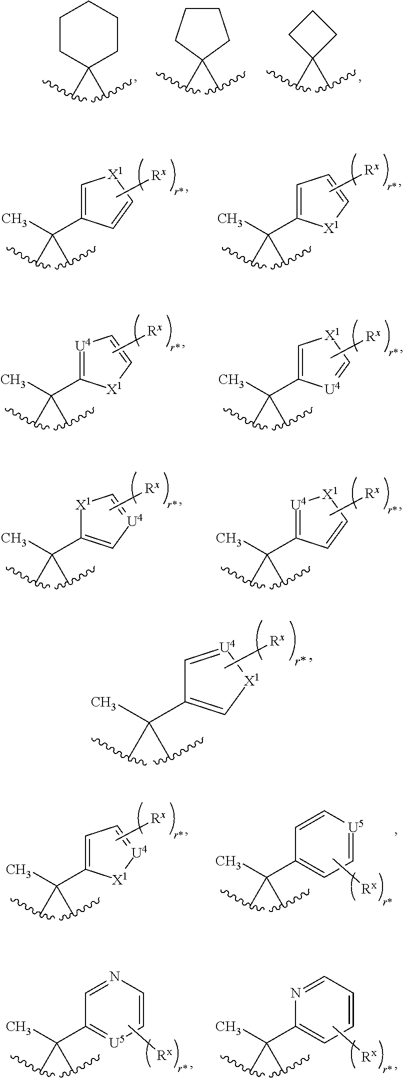

wherein
U⁴ and U⁵ are each independently N or CH
X¹ is NH, O or S(O)ᵥ
r* is 0 or an integer up to three as allowed by valance.

Preferred compounds of the present invention further include compounds wherein R⁴ is

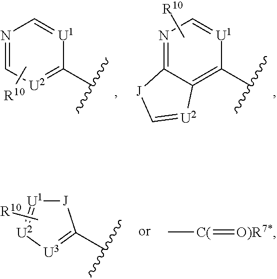

any of which may be optionally substituted, as allowed by valance, with up to three R^{x} groups independently selected from halo, cyano, alkyl, haloalkyl, alkenyl, alkynyl, aryl, -(alkylene)_{q}-NR⁸⁺R⁹⁺, -(alkylene)_{q}-C(═O)NR⁸⁺R⁹⁺, -(alkylene)_{q}-OR⁷⁺,
wherein R¹⁰ is H, —NR¹⁴R¹⁵, or —C(═O)NR¹⁴R¹⁵; and
U¹, U² and U³ are independently CH or N.

Preferred compounds of the present invention further include compounds wherein R⁴ is

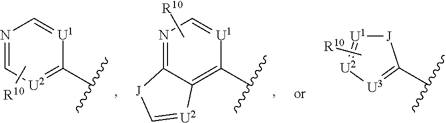

any of which may be optionally substituted, as allowed by valance, with up to three R^{x} groups independently selected from halo, cyano, alkyl, haloalkyl, alkenyl, alkynyl, aryl, -(alkylene)_{q}-NR⁸⁺R⁹⁺, -(alkylene)_{q}-C(═O)NR⁸⁺R⁹⁺, -(alkylene)_{q}-OR⁷⁺,
wherein R¹⁰ is H, —NR¹⁴R¹⁵, or —C(═O)NR¹⁴R¹⁵; and
U¹, U² and U³ are independently CH or N.

Preferred compounds of the present invention include compounds wherein R⁴ is

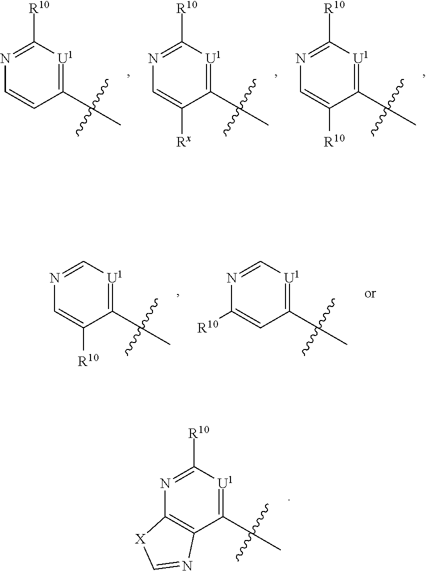

Preferred compounds of the present invention include compounds having the following formula IA

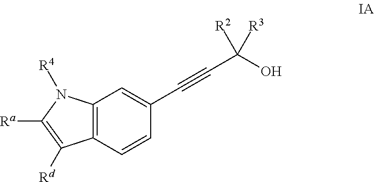

Preferred compounds of the present invention further include compounds having the following formula IB

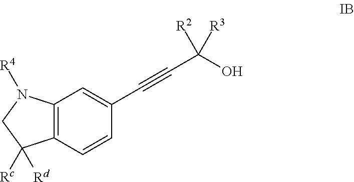

Preferred compounds of the present invention further include compounds having the following formula IC

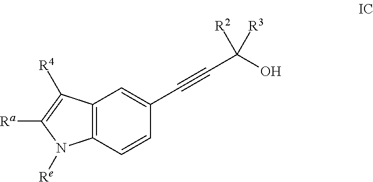

Preferred compounds of the present invention also include compounds having any and all combinations and sub-combinations of the preferred groups listed above.

Preferred compounds of the present invention include the compounds exemplified herein.

The present invention also relates to pharmaceutical compositions containing the above compounds, together with a pharmaceutically acceptable vehicle or carrier.

The present invention also relates to a method of treating inflammation and/or inflammatory disorders in a subject using the above compounds.

The invention also relates to a method of treating NIK-mediated disorders in a subject using the above compounds.

### Indications

Compounds of the present invention are useful in the treatment of inflammatory and autoimmune disorders, including RA and inflammatory bowel diseases (IBD), asthma, COPD, multiple sclerosis, colitis, arthritis, and insulin-dependent diabetes mellitus. Compounds of the present invention may also be useful in treating lymphoma, leukemia and multiple myeloma.

### DEFINITIONS

The terms “agonist” and “agonistic” when used herein refer to or describe a molecule which is capable of, directly or indirectly, substantially inducing, promoting or enhancing HGF biological activity or HGF receptor activation.

The terms “treating,” “treatment,” and “therapy” as used herein refer to curative therapy, prophylactic therapy, and preventative therapy.

The term “mammal” as used herein refers to any mammal classified as a mammal, including humans, cows, horses, dogs and cats. In a preferred embodiment of the invention, the mammal is a human.

The term “treatment” includes therapeutic treatment as well as prophylactic treatment (either preventing the onset of disorders altogether or delaying the onset of a pre-clinically evident stage of disorders in individuals).

A “pharmaceutically-acceptable derivative” denotes any salt, ester of a compound of this invention, or any other compound which upon administration to a patient is capable of providing (directly or indirectly) a compound of this invention, or a metabolite or residue thereof, characterized by the ability to inhibit angiogenesis.

The phrase “therapeutically-effective” is intended to qualify the amount of each agent, which will achieve the goal of improvement in disorder severity and the frequency of incidence over treatment of each agent by itself, while avoiding adverse side effects typically associated with alternative therapies. For example, effective neoplastic therapeutic agents prolong the survivability of the patient, inhibit the rapidly proliferating cell growth associated with the neoplasm, or effect a regression of the neoplasm.

The term “H” denotes a single hydrogen atom. This radical may be attached, for example, to an oxygen atom to form a hydroxyl radical.

Where the term “alkyl” is used, either alone or within other terms such as “haloalkyl” and “alkylamino”, it embraces linear or branched radicals having one to about twelve carbon atoms. More preferred alkyl radicals are “lower alkyl” radicals having one to about six carbon atoms. Examples of such radicals include methyl, ethyl, n-propyl, isopropyl, n-butyl, isobutyl, sec-butyl, tert-butyl, pentyl, isoamyl, hexyl and the like. Even more preferred are lower alkyl radicals having one or two carbon atoms. The term “alkylenyl” embraces bridging divalent alkyl radicals such as methylenyl and ethylenyl. The term “lower alkyl substituted with R²” does not include an acetal moiety.

The term “alkenyl” embraces linear or branched radicals having at least one carbon-carbon double bond of two to about twelve carbon atoms. More preferred alkenyl radicals are “lower alkenyl” radicals having two to about six carbon atoms. Most preferred lower alkenyl radicals are radicals having two to about four carbon atoms. Examples of alkenyl radicals include ethenyl, propenyl, allyl, propenyl, butenyl and 4-methylbutenyl. The terms “alkenyl” and “lower alkenyl”, embrace radicals having “cis” and “trans” orientations, or alternatively, “E” and “Z” orientations.

The term “alkynyl” denotes linear or branched radicals having at least one carbon-carbon triple bond and having two to about twelve carbon atoms. More preferred alkynyl radicals are “lower alkynyl” radicals having two to about six carbon atoms. Most preferred are lower alkynyl radicals having two to about four carbon atoms. Examples of such radicals include propargyl, butynyl, and the like.

Alkyl, alkylenyl, alkenyl, and alkynyl radicals may be optionally substituted with one or more functional groups such as halo, hydroxy, nitro, amino, cyano, haloalkyl, aryl, heteroaryl, heterocyclo and the like.

The term “halo” means halogens such as fluorine, chlorine, bromine or iodine atoms.

The term “haloalkyl” embraces radicals wherein any one or more of the alkyl carbon atoms is substituted with halo as defined above. Specifically embraced are monohaloalkyl, dihaloalkyl and polyhaloalkyl radicals including perhaloalkyl. A monohaloalkyl radical, for one example, may have either an iodo, bromo, chloro or fluoro atom within the radical. Dihalo and polyhaloalkyl radicals may have two or more of the same halo atoms or a combination of different halo radicals. “Lower haloalkyl” embraces radicals having 1-6 carbon atoms. Even more preferred are lower haloalkyl radicals having one to three carbon atoms. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perfluoroalkyl” means alkyl radicals having all hydrogen atoms replaced with fluoro atoms. Examples include trifluoromethyl and pentafluoroethyl.

The term “hydroxyalkyl” embraces linear or branched alkyl radicals having one to about ten carbon atoms any one of which may be substituted with one or more hydroxyl radicals. More preferred hydroxyalkyl radicals are “lower hydroxyalkyl” radicals having one to six carbon atoms and one or more hydroxyl radicals. Examples of such radicals include hydroxymethyl, hydroxyethyl, hydroxypropyl, hydroxybutyl and hydroxyhexyl. Even more preferred are lower hydroxyalkyl radicals having one to three carbon atoms.

The term “alkoxy” embraces linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy. Even more preferred are lower alkoxy radicals having one to three carbon atoms. Alkoxy radicals may be further substituted with one or more halo atoms, such as fluoro, chloro or bromo, to provide “haloalkoxy” radicals. Even more preferred are lower haloalkoxy radicals having one to three carbon atoms. Examples of such radicals include fluoromethoxy, chloromethoxy, trifluoromethoxy, trifluoroethoxy, fluoroethoxy and fluoropropoxy.

The term “aryl”, alone or in combination, means a carbocyclic aromatic system containing one or two rings wherein such rings may be attached together in a fused manner. The term “aryl” embraces aromatic radicals such as phenyl, naphthyl, indenyl, tetrahydronaphthyl, and indanyl. More preferred aryl is phenyl. Said “aryl” group may have 1 or more substituents such as lower alkyl, hydroxyl, halo, haloalkyl, nitro, cyano, alkoxy, lower alkylamino, and the like. Phenyl substituted with —O—CH₂—O— forms the aryl benzodioxolyl substituent.

The term “heterocyclyl” (or “heterocyclo”) embraces saturated, and partially saturated and heteroatom-containing ring radicals, where the heteroatoms may be selected from nitrogen, sulfur and oxygen. It does not include rings containing —O—O—, —O—S— or —S—S— portions. Said “heterocyclyl” group may have 1 to 3 substituents such as hydroxyl, Boc, halo, haloalkyl, cyano, lower alkyl, lower aralkyl, oxo, lower alkoxy, amino, lower alkylamino, and the like.

Examples of saturated heterocyclic radicals include saturated 3 to 6-membered heteromonocyclic groups containing 1 to 4 nitrogen atoms [e.g. pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocyclyl radicals include dihydrothienyl, dihydropyranyl, dihydrofuryl, dihydrothiazolyl, and the like.

Particular examples of partially saturated and saturated heterocyclyl include pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl, and the like.

The term heterocyclyl, (or heterocyclo) also embraces radicals where heterocyclic radicals are fused/condensed with aryl radicals: unsaturated condensed heterocyclic group containing 1 to 5 nitrogen atoms, for example, indolyl, isoindolyl, indolizinyl, benzimidazolyl, quinolyl, isoquinolyl, indazolyl, benzotriazolyl, tetrazolopyridazinyl [e.g., tetrazolo[1,5-b]pyridazinyl]; unsaturated condensed heterocyclic group containing 1 to 2 oxygen atoms and 1 to 3 nitrogen atoms [e.g. benzoxazolyl, benzoxadiazolyl]; unsaturated condensed heterocyclic group containing 1 to 2 sulfur atoms and 1 to 3 nitrogen atoms [e.g., benzothiazolyl, benzothiadiazolyl]; and saturated, partially unsaturated and unsaturated condensed heterocyclic group containing 1 to 2 oxygen or sulfur atoms [e.g. benzofuryl, benzothienyl, 2,3-dihydro-benzo[1,4]dioxinyl and dihydrobenzofuryl].

The term “heteroaryl” denotes aryl ring systems that contain one or more heteroatoms selected from the group O, N and S, wherein the ring nitrogen and sulfur atom(s) are optionally oxidized, and nitrogen atom(s) are optionally quarternized. Examples include unsaturated 5 to 6 membered heteromonocyclyl group containing 1 to 4 nitrogen atoms, for example, pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, 1H-1,2,3-triazolyl, 2H-1,2,3-triazolyl]; unsaturated 5- to 6-membered heteromonocyclic group containing an oxygen atom, for example, pyranyl, 2-furyl, 3-furyl, etc.; unsaturated 5 to 6-membered heteromonocyclic group containing a sulfur atom, for example, 2-thienyl, 3-thienyl, etc.; unsaturated 5- to 6-membered heteromonocyclic group containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic group containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl].

The term “sulfonyl”, whether used alone or linked to other terms such as alkylsulfonyl, denotes respectively divalent radicals —SO₂—.

The terms “sulfamyl,” “aminosulfonyl” and “sulfonamidyl,” denotes a sulfonyl radical substituted with an amine radical, forming a sulfonamide (—SO₂NH₂).

The term “alkylaminosulfonyl” includes “N-alkylaminosulfonyl” where sulfamyl radicals are independently substituted with one or two alkyl radical(s). More preferred alkylaminosulfonyl radicals are “lower alkylaminosulfonyl” radicals having one to six carbon atoms. Even more preferred are lower alkylaminosulfonyl radicals having one to three carbon atoms. Examples of such lower alkylaminosulfonyl radicals include N-methylaminosulfonyl, and N-ethylaminosulfonyl.

The terms “carboxy” or “carboxyl”, whether used alone or with other terms, such as “carboxyalkyl”, denotes —CO₂H.

The term “carbonyl”, whether used alone or with other terms, such as “aminocarbonyl”, denotes —(C═O)—.

The term “aminocarbonyl” denotes an amide group of the formula —C(═O)NH₂.

The terms “N-alkylaminocarbonyl” and “N,N-dialkylaminocarbonyl” denote aminocarbonyl radicals independently substituted with one or two alkyl radicals, respectively. More preferred are “lower alkylaminocarbonyl” having lower alkyl radicals as described above attached to an aminocarbonyl radical.

The terms “N-arylaminocarbonyl” and “N-alkyl-N-arylaminocarbonyl” denote aminocarbonyl radicals substituted, respectively, with one aryl radical, or one alkyl and one aryl radical.

The terms “heterocyclylalkylenyl” and “heterocyclylalkyl” embrace heterocyclic-substituted alkyl radicals. More preferred heterocyclylalkyl radicals are “5- or 6-membered heteroarylalkyl” radicals having alkyl portions of one to six carbon atoms and a 5- or 6-membered heteroaryl radical. Even more preferred are lower heteroarylalkylenyl radicals having alkyl portions of one to three carbon atoms. Examples include such radicals as pyridylmethyl and thienylmethyl.

The term “aralkyl” embraces aryl-substituted alkyl radicals. Preferable aralkyl radicals are “lower aralkyl” radicals having aryl radicals attached to alkyl radicals having one to six carbon atoms. Even more preferred are “phenylalkylenyl” attached to alkyl portions having one to three carbon atoms. Examples of such radicals include benzyl, diphenylmethyl and phenylethyl. The aryl in said aralkyl may be additionally substituted with halo, alkyl, alkoxy, halkoalkyl and haloalkoxy.

The term “alkylthio” embraces radicals containing a linear or branched alkyl radical, of one to ten carbon atoms, attached to a divalent sulfur atom. Even more preferred are lower alkylthio radicals having one to three carbon atoms. An example of “alkylthio” is methylthio, (CH₃S—).

The term “haloalkylthio” embraces radicals containing a haloalkyl radical, of one to ten carbon atoms, attached to a divalent sulfur atom. Even more preferred are lower haloalkylthio radicals having one to three carbon atoms. An example of “haloalkylthio” is trifluoromethylthio.

The term “alkylamino” embraces “N-alkylamino” and “N,N-dialkylamino” where amino groups are independently substituted with one alkyl radical and with two alkyl radicals, respectively. More preferred alkylamino radicals are “lower alkylamino” radicals having one or two alkyl radicals of one to six carbon atoms, attached to a nitrogen atom. Even more preferred are lower alkylamino radicals having one to three carbon atoms. Suitable alkylamino radicals may be mono or dialkylamino such as N-methylamino, N-ethylamino, N,N-dimethylamino, N,N-diethylamino and the like.

The term “arylamino” denotes amino groups, which have been substituted with one or two aryl radicals, such as N-phenylamino. The arylamino radicals may be further substituted on the aryl ring portion of the radical.

The term “heteroarylamino” denotes amino groups, which have been substituted with one or two heteroaryl radicals, such as N-thienylamino. The “heteroarylamino” radicals may be further substituted on the heteroaryl ring portion of the radical.

The term “aralkylamino” denotes amino groups, which have been substituted with one or two aralkyl radicals. More preferred are phenyl-C₁-C₃-alkylamino radicals, such as N-benzylamino. The aralkylamino radicals may be further substituted on the aryl ring portion.

The terms “N-alkyl-N-arylamino” and “N-aralkyl-N-alkylamino” denote amino groups, which have been independently substituted with one aralkyl and one alkyl radical, or one aryl and one alkyl radical, respectively, to an amino group.

The term “aminoalkyl” embraces linear or branched alkyl radicals having one to about ten carbon atoms any one of which may be substituted with one or more amino radicals. More preferred aminoalkyl radicals are “lower aminoalkyl” radicals having one to six carbon atoms and one or more amino radicals. Examples of such radicals include aminomethyl, aminoethyl, aminopropyl, aminobutyl and aminohexyl. Even more preferred are lower aminoalkyl radicals having one to three carbon atoms.

The term “alkylaminoalkyl” embraces alkyl radicals substituted with alkylamino radicals. More preferred alkylaminoalkyl radicals are “lower alkylaminoalkyl” radicals having alkyl radicals of one to six carbon atoms. Even more preferred are lower alkylaminoalkyl radicals having alkyl radicals of one to three carbon atoms. Suitable alkylaminoalkyl radicals may be mono or dialkyl substituted, such as N-methylaminomethyl, N,N-dimethyl-aminoethyl, N,N-diethylaminomethyl and the like.

The term “alkylaminoalkoxy” embraces alkoxy radicals substituted with alkylamino radicals. More preferred alkylaminoalkoxy radicals are “lower alkylaminoalkoxy” radicals having alkoxy radicals of one to six carbon atoms. Even more preferred are lower alkylaminoalkoxy radicals having alkyl radicals of one to three carbon atoms. Suitable alkylaminoalkoxy radicals may be mono or dialkyl substituted, such as N-methylaminoethoxy, N,N-dimethylaminoethoxy, N,N-diethylaminoethoxy and the like.

The term “alkylaminoalkoxyalkoxy” embraces alkoxy radicals substituted with alkylaminoalkoxy radicals. More preferred alkylaminoalkoxyalkoxy radicals are “lower alkylaminoalkoxyalkoxy” radicals having alkoxy radicals of one to six carbon atoms. Even more preferred are lower alkylaminoalkoxyalkoxy radicals having alkyl radicals of one to three carbon atoms. Suitable alkylaminoalkoxyalkoxy radicals may be mono or dialkyl substituted, such as N-methylaminomethoxyethoxy, N-methylaminoethoxyethoxy, N,N-dimethylaminoethoxyethoxy, N,N-diethylaminomethoxymethoxy and the like.

The term “carboxyalkyl” embraces linear or branched alkyl radicals having one to about ten carbon atoms any one of which may be substituted with one or more carboxy radicals. More preferred carboxyalkyl radicals are “lower carboxyalkyl” radicals having one to six carbon atoms and one carboxy radical. Examples of such radicals include carboxymethyl, carboxypropyl, and the like. Even more preferred are lower carboxyalkyl radicals having one to three CH₂ groups.

The term “halosulfonyl” embraces sulfonyl radicals substituted with a halogen radical. Examples of such halosulfonyl radicals include chlorosulfonyl and fluorosulfonyl.

The term “arylthio” embraces aryl radicals of six to ten carbon atoms, attached to a divalent sulfur atom. An example of “arylthio” is phenylthio.

The term “aralkylthio” embraces aralkyl radicals as described above, attached to a divalent sulfur atom. More preferred are phenyl-C₁-C₃-alkylthio radicals. An example of “aralkylthio” is benzylthio.

The term “aryloxy” embraces optionally substituted aryl radicals, as defined above, attached to an oxygen atom. Examples of such radicals include phenoxy.

The term “aralkoxy” embraces oxy-containing aralkyl radicals attached through an oxygen atom to other radicals. More preferred aralkoxy radicals are “lower aralkoxy” radicals having optionally substituted phenyl radicals attached to lower alkoxy radical as described above.

The term “heteroaryloxy” embraces optionally substituted heteroaryl radicals, as defined above, attached to an oxygen atom.

The term “heteroarylalkoxy” embraces oxy-containing heteroarylalkyl radicals attached through an oxygen atom to other radicals. More preferred heteroarylalkoxy radicals are “lower heteroarylalkoxy” radicals having optionally substituted heteroaryl radicals attached to lower alkoxy radical as described above.

The term “cycloalkyl” includes saturated carbocyclic groups. Preferred cycloalkyl groups include C₃-C₆ rings. More preferred compounds include, cyclopentyl, cyclopropyl, and cyclohexyl.

The term “cycloalkylalkyl” embraces cycloalkyl-substituted alkyl radicals. Preferable cycloalkylalkyl radicals are “lower cycloalkylalkyl” radicals having cycloalkyl radicals attached to alkyl radicals having one to six carbon atoms. Even more preferred are “5-6-membered cycloalkylalkyl” attached to alkyl portions having one to three carbon atoms. Examples of such radicals include cyclohexylmethyl. The cycloalkyl in said radicals may be additionally substituted with halo, alkyl, alkoxy and hydroxy.

The term “cycloalkenyl” includes carbocyclic groups having one or more carbon-carbon double bonds including “cycloalkyldienyl” compounds. Preferred cycloalkenyl groups include C₃-C₆ rings. More preferred compounds include, for example, cyclopentenyl, cyclopentadienyl, cyclohexenyl and cycloheptadienyl.

The term “comprising” is meant to be open ended, including the indicated component but not excluding other elements.

The term(s) “Formulas I, II, III, IV, V, VI and VII” either alone or in combination includes any sub formulas.

The compounds of the invention are endowed with c-Met inhibitory activity.

The present invention also comprises the use of a compound of the invention, or pharmaceutically acceptable salt thereof, in the manufacture of a medicament for the treatment either acutely or chronically of an angiogenesis mediated disease state, including those described previously. The compounds of the present invention are useful in the manufacture of an anti-cancer medicament. The compounds of the present invention are also useful in the manufacture of a medicament to attenuate or prevent disorders through inhibition of c-Met.

The present invention comprises a pharmaceutical composition comprising a therapeutically effective amount of a compound of the current invention in association with a least one pharmaceutically acceptable carrier, adjuvant or diluent.

The present invention also comprises a method of treating angiogenesis related disorders in a subject having or susceptible to such disorder, the method comprising treating the subject with a therapeutically effective amount of a compound of the current invention.

### Combinations

While the compounds of the invention can be administered as the sole active pharmaceutical agent, they can also be used in combination with one or more compounds of the invention or other agents. When administered as a combination, the therapeutic agents can be formulated as separate compositions that are administered at the same time or sequentially at different times, or the therapeutic agents can be given as a single composition.

The phrase “co-therapy” (or “combination-therapy”), in defining use of a compound of the present invention and another pharmaceutical agent, is intended to embrace administration of each agent in a sequential manner in a regimen that will provide beneficial effects of the drug combination, and is intended as well to embrace co-administration of these agents in a substantially simultaneous manner, such as in a single capsule having a fixed ratio of these active agents or in multiple, separate capsules for each agent.

Specifically, the administration of compounds of the present invention may be in conjunction with additional therapies known to those skilled in the art in the prevention or treatment of inflammatory disorders.

If formulated as a fixed dose, such combination products employ the compounds of this invention within the accepted dosage ranges.

Also included in the family of compounds of the current are the pharmaceutically acceptable salts and solvates thereof. The term “pharmaceutically-acceptable salts” embraces salts commonly used to form alkali metal salts and to form addition salts of free acids or free bases. The nature of the salt is not critical, provided that it is pharmaceutically acceptable. Suitable pharmaceutically acceptable acid addition salts of compounds of the current invention may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, arylaliphatic, heterocyclic, carboxylic and sulfonic classes of organic acids, example of which are formic, acetic, adipic, butyric, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, ethanedisulfonic, benzenesulfonic, pantothenic, 2-hydroxyethanesulfonic, toluenesulfonic, sulfanilic, cyclohexylaminosulfonic, camphoric, camphorsulfonic, digluconic, cyclopentanepropionic, dodecylsulfonic, glucoheptanoic, glycerophosphonic, heptanoic, hexanoic, 2-hydroxy-ethanesulfonic, nicotinic, 2-naphthalenesulfonic, oxalic, palmoic, pectinic, persulfuric, 2-phenylpropionic, picric, pivalic propionic, succinic, tartaric, thiocyanic, mesylic, undecanoic, stearic, algenic, β-hydroxybutyric, salicylic, galactaric and galacturonic acid. Suitable pharmaceutically-acceptable base addition salts of compounds of the current invention include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, aistidine, glucamine, isopropylamine, lysine, morpholine, N-ethyl morpholine, piperazine, piperidine, triethylamine, trimethylamine. All of these salts may be prepared by conventional means from the corresponding compound of the invention by reacting, for example, the appropriate acid or base with the compound of the current invention. When a basic group and an acid group are present in the same molecule, a compound of the current invention may also form internal salts.

### General Synthetic Procedures

The following is a key of abbreviations which may appear in the specification:
HOAc—acetic acid
MeCN, CH₃CN—acetonitrile
NH₃—ammonia
NH₄Cl—ammonium chloride
Ar—argon
HBTA—O-benzotriazol-1-yl-N,N,N′,N′-tetramethyluronium hexafluorophosphate
HATU—O-(7-azabenzotriazol-1-yl)-N,N,N′,N′-tetramethyluronium hexafluorophosphate
PyBop—benzotriazol-1-yl-oxy-tripyrrolidino-phosphonium hexafluorophosphate
Pd₂(dba)₃—bis(dibenzylideneacetone) palladium
BINAP—2,2′-bis(diphenylphosphino)-1,1′-binaphthyl
TEAC—bis(tetra-ethylammonium)carbonate
BBr₃—boron tribromide
BSA—bovine serum albumin
Br₂—bromine
BOC—butyloxycarbonyl
Cs₂CO₃—cesium carbonate
CHCl₃—chloroform
CDCl₃—chloroform deuterated
Cu—copper
CuI—copper(I) iodide
Et₂O—diethyl ether
DBU—1,8-diazabicyclo[5.4.0]undec-7-ene
DIBAL—diisobutylaluminum hydride
DIAD—diisopropyl azodicarboxylate
DIEA—diisopropylethylamine
DMF—dimethylformamide
DMAP—4-dimethylaminopyridine
DMSO—dimethylsulfoxide
EDC, EDCI—1-(3-dimethylaminopropyl)-3-ethylcarbodiimide hydrochloride
dppa—diphenylphosphoryl azide
EtOAc—ethyl acetate
FBS—fetal bovine serum
g—gram
h—hour
HBr—hydrobromic acid
HCl—hydrochloric acid
HOBt—1-hydroxybenzotriazole hydrate
H₂—hydrogen
H₂O₂-hydrogen peroxide
Fe—iron
LiHMDS—lithium bis(trimethylsilyl)-amide
LDA—Lithium diisopropylamide
MCPBA—meta-chloroperbenzoic acid
MgSO₄—magnesium sulfate
MeOH, CH₃OH—methanol
MeI—methyl iodide
CH₂Cl₂, DCM—methylene chloride
NMP—N-methylpyrrolidinone
ML, ml—milliliter
N₂—nitrogen
Pd/C—palladium on carbon
Pd(OAc)₂—palladium acetate
Pd(OH)₂—palladium hydroxide
Pd(PPh₃)₄—palladium tetrakis triphenylphosphine
Pd(dppf)Cl₂—1,1-bis(diphenylphosphino)ferrocene palladium chloride
PBS—phosphate buffered saline
POCl₃—phosphorous oxychloride
K₂CO₃—potassium carbonate
KOH—potassium hydroxide
RT—room temperature
NaHCO₃—sodium bicarbonate
NaBH₄—sodium borohydride
NaBH₃CN—sodium cyanoborohydride
NaOtBu—sodium tert-butoxide
NaOH—sodium hydroxide
NaClO₂—sodium chlorite
NaCl—sodium chloride
NaHPO₄—sodium biphospate
NaH—sodium hydride
NaI—sodium iodide
Na₂SO₄—sodium sulfate
TBTU—O-benzotriazol-1-yl-N,N,N′,N′-tetramethyluronium tetrafluoroborate
THF—tetrahydrofuran
Et₃N, TEA—triethylamine
TFA—trifluoroacetic acid
P(t-bu)₃—tri(tert-butyl)phosphine
H₂O—water

Compounds of the current invention may be synthesized according to the schemes illustrated in the following working examples, as well as through the schemes illustrated in the General Schemes Sections, and other methods known to those of skill in the art.

### General Schemes—Section A

### Building Blocks and Precursors

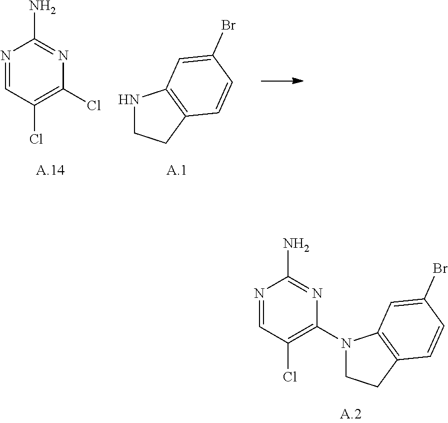

4-(6-bromoindolin-1-yl)-5-chloropyrimidin-2-amine (A.2). To a stirred solution of 4,5-dichloropyrimidin-2-amine (A.14) (1.8 g, 11 mmol) and 6-bromoindoline (A.1)(2.18 g, 1 eq) in dioxane (25 mL) was added HCl (1.25 M in EtOH, 11.4 mL) and the mixture was heated to 90° C. for 18 hrs. The reaction cooled to room temp and the solvent evaporated. The residue was suspended in 50 mL NH₄OH, and the precipitate was collected by filtration. Purification by flash chromatography on silica (10% 89:9:1 dichloromethane-methanol-NH₄OH in dichloromethane) yielded 1.86 g 4-(6-bromoindolin-1-yl)-5-chloropyrimidin-2-amine (A.2).

1H NMR (500 MHz, CHLOROFORM-d) δ ppm 8.07 (1H, s), 7.69 (1H, s), 7.09 (1H, dd, J=8.0, 2.0 Hz), 7.07 (1H, d, J=8.0 Hz), 5.08 (2H, s), 4.35 (2H, t, J=8.3 Hz), 3.11 (2H, t, J=8.3 Hz) Mass Spectrum (ESI) m/e=325.5 and 327.0 (M+1).

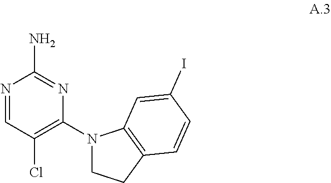

5-chloro-4-(6-iodoindolin-1-yl)pyrimidin-2-amine A.3 4-(6-bromoindolin-1-yl)-5-chloropyrimidin-2-amine A.2 (1.25 g, 3.8 mmol), CuI (0.438 g, 2.3 mmol) and NaI (1.14 g, 7.6 mmol) were added to a Schlenk tube, evacuated and backfilled with argon. Under positive pressure of argon, the screw cap was replaced with a septum. N,N′-dimethylethylenediamine (0.41 mL, 3.8 mmol) in dioxane (5 mL) was added via syringe, and the screwcap was returned under positive pressure of argon. The reaction was sealed and heated to 110° C. with stirring for 20 hours. The mixture was cooled to room temperature, diluted with 15 mL 5% aq. NH₃, and poured into 100 mL water. The solution was extracted with dichloromethane and the combined extracts were dried over MgSO₄, filtered and evaporated. Purification by flash chromatography (30% ethyl acetate in hexane) gave 0.86 g 5-chloro-4-(6-iodoindolin-1-yl)pyrimidin-2-amine A.3. Mass Spectrum (ESI) m/e=373.0 (M+1)

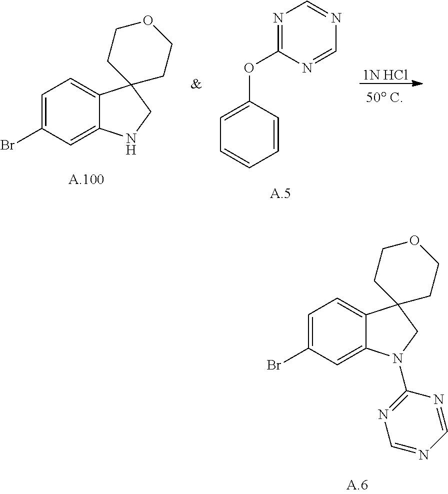

### 6-bromo-1-(1,3,5-triazin-2-yl)-1,2,2′,3′,5′,6′-hexahydrospiro[indole-3,4′-pyran] A.6

6-bromo-1,2,2′,3′,5′,6′-hexahydrospiro[indole-3,4′-pyran] (177 mg, 0.660 mmol) A.100, and 2-phenoxy-1,3,5-triazine A.5 (171 mg, 0.990 mmol) (R. Hirt, H. Nidecker, et al. (1950). Helvetica Chimica Acta 33(5): 1365-1369.), were added to a solution of isopropanol (5 ml) and 1N HCl (1 ml). The solution was heated to 50° C. for 1 hr before being cooled to room temperature. After diluting the solution with saturated NaHCO3 it was extracted with ethyl acetate. The organics were dried over MgSO₄ before being concentrated under vacuum. The residue obtained was enriched by chromatography on silica eluting with 0.5% triethylamine/5% methanol/dichloromethane. The fractions containing the product were combined and concentrated under vacuum. The residue obtained was diluted with ethyl acetate and washed with water. The organics were dried over MgSO4 and then concentrated under vacuum. 6-bromo-1-(1,3,5-triazin-2-yl)-1,2,2′,3′,5′,6′-hexahydrospiro[indole-3,4′-pyran] A.6 (170 mg) was obtained as a brownish solid.

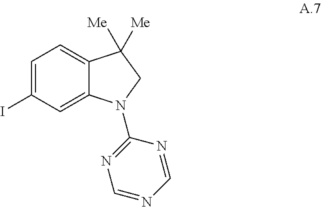

6-iodo-3,3-dimethyl-1-(1,3,5-triazin-2-yl)-2,3-dihydro-1H-indole A.7 was synthesized from A.10 by the procedure used to prepare A.6; the title compound was obtained as a brownish solid (117 mg).

ESI-MS: M+H⁺ 352.9 m/z.

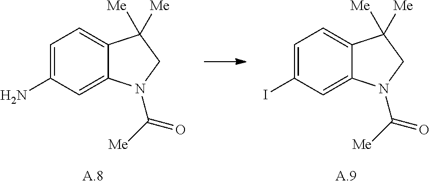

### 1-acetyl-6-iodo-3,3-dimethyl-2,3-dihydro-1H-indole A.9

In a 500 ml three-necked round bottom flask equipped with an overhead stirrer, was combined 1-acetyl-3,3-dimethyl-2,3-dihydro-1H-indol-6-amine A.8 (6.98 g, 34.22 mmol) [Chen, G., J. Adams, et al. WO 2002066470 p 263] with 30 ml of ice/water. The solution was cooled in an ice bath before concentrated HCl (6.8 ml, 81.60 mmol) was added. A solution of NaNO₂ (2.48 g, 35.93 mmol) dissolved in 30 ml of water was added drop wise over a period of 10 minutes. After 30 minutes a solution of KI (11.36 g, 68.44 mmol) dissolved in 70 ml of chloroform was added via an addition funnel over a period of 0.5 hr. Then the brownish solution was stirred at room temperature until gas evolution ceased. Partitioned the solution in a separation funnel, and washed the organic layer with saturated NaHCO₃, followed by 5% Na₂S₂O₃. The organics were dried over MgSO₄ before being concentrated under vacuum to 1/10^{th} the volume. Purified the residue obtained by chromatography on silica eluting with 20% hexane/dichloromethane. The fractions containing the product were combined and concentrated under vacuum to give 1-acetyl-6-iodo-3,3-dimethyl-2,3-dihydro-1H-indole A.9 (6.95 g, 65%) as a tan colored solid.

¹H NMR (500 MHz, (CD)₃SO) δ 8.39 (s, 1H), 7.37 (d, J=7.8 Hz, 1H), 7.08 (d, J=7.8 Hz, 1H), 3.85 (s, 2H), 2.16 (s, 3H), 1.29 (s, 6H); ESI MS: M+H⁺ 316.0 m/z.

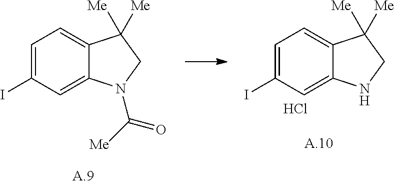

### 6-iodo-3,3-dimethyl-2,3-dihydro-1H-indole HCl A.10

1-acetyl-6-iodo-3,3-dimethyl-2,3-dihydro-1H-indole A.9 (6.95 g, 22.08 mmol) was combined with methanol and concentrated HCl (25 ml, 300 mmol). The solution was heated at a gentle reflux for 1 hr before it was cooled to room temperature. After cooling the solution to 0° C. a white solid was collected by filtration to afford 6-iodo-3,3-dimethyl-2,3-dihydro-1H-indole A.10 as the HCl salt (5.96 g, 86%).

ESI MS: M+H⁺ 274.0 m/z.

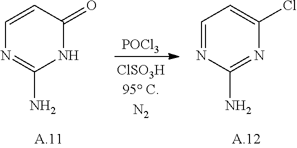

### 4-chloro-2-pyrimidinamine A.12

In a three neck 1 L round bottom flask equipped with reflux condenser was added 2-amino-4(3H)-pyrimidinone A.11 (100 g, 0.9 mol) (available from Toronto Research Chemicals) followed by POCl₃ (168 ml, 1.800 mol) at room temperature and under an atmosphere of N₂. To this was cautiously added ClSO₃H (4.8 ml, 72.01 mmol). The solution was heated to 95° C. for 4 hrs, before it was cooled to room temperature. The solution was then cooled in an ice bath before it was poured into 700 ml of ice water with vigorous stirring. Adjusted the pH to ˜7 with NH₄OH (30% by weight) (temperature was held below 20° C.). A tan colored solid was collected by filtration. The solid was dried under vacuum at 70° C. overnight to afford 4-chloro-2-pyrimidinamine A.12 (108 g, 92%) as an off white solid.

¹H NMR (400 MHz, (CD)₃SO) δ 8.17 (d, J=5.2 Hz, 1H), 7.07 (br s, 2H), 6.64 (d, J=5.2 Hz, 1H); ESI MS: M+H⁺ 130.0 m/z.

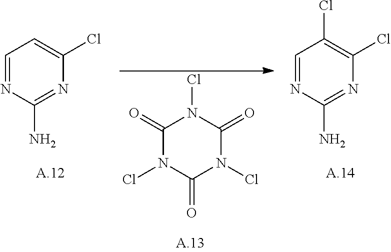

### 4,5-dichloro-2-pyrimidinamine A.14

4-chloro-2-pyrimidinamine (50 g, 450 mmol) A.12 was combined with trichloroisocyanuric acid A.13 (52 g, 225 mmol) in 450 ml of water, and 50 ml of acetic acid. The reaction was heated to 50° C. with stirring for 20 hrs. The solution was then cooled to room temperature before it was transferred into a 4 L beaker. The pH of the solution was adjusted to ˜6 with 10N NaOH (the temperature was held below 20° C. by adding ice). The pH was then adjusted to ˜7-8 with NaHCO₃. After stirring the solution for 4 hrs a pink solid was filtered off and washed with water. The pink solid was added to 1 L of water and stirred at room temperature for 1 hr. The solid was collected by filtration, washed with water and dried under vacuum at 60° C. to give 4,5-dichloro-2-pyrimidinamine A.14 (48 g).

¹H NMR (500 MHz, (CD)₃SO) δ 8.37 (s, 1H), 7.33 (br s, 2H); ESI MS: M+H⁺ 164.9 m/z.

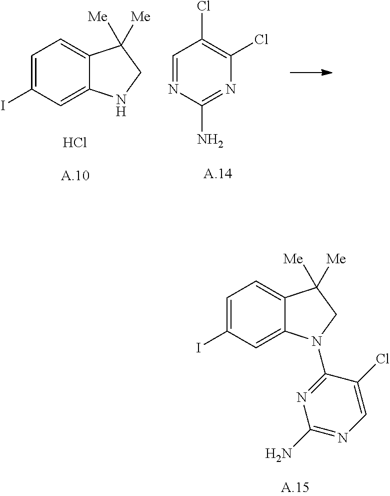

5-chloro-4-(6-iodo-3,3-dimethyl-2,3-dihydro-1H-indol-1-yl)-2-pyrimidinamine A.15 This compound was prepared from A.10 and A.14 by the procedure used to prepare A.2

¹H NMR (500 MHz, (DMSO-d6) δ 8.12 (s, 1H), 7.52 (d, J=1.5 Hz, 1H), 7.27 (dd, J=7.9 Hz, J=1.5 Hz, 1H), 7.03 (d, J=7.8 Hz, 1H), 6.69 (br s, 2H), 3.91 (s, 2H), 1.27 (s, 6H); ESI MS: M+H⁺401.2 m/z.

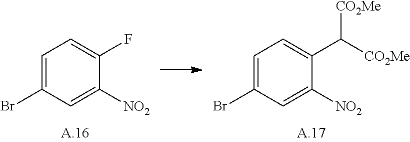

Dimethyl 2-(4-bromo-2-nitrophenyl)malonate A.17 was prepared by the method of Quallich, G. J. and P. M. Morrissey (1993). Synthesis 51-53.

To a stirred ice-cooled solution of dimethyl malonate (44.12 g, 1.5 eq.) in dry DMF (200 mL) was added K₂CO₃ powder (90.5 g, 3 eq.) followed by 4-bromo-1-fluoro-2-nitrobenzene A.16 (47.5 g, 218.2 mmol) through a syringe. The resulting mixture was stirred at ambient temperature for 24 h and then poured into ice and 2 N HCl aqueous solution. More aqueous 2 N HCl was added until the precipitation was complete. The precipitate was then collected by vacuum filtration, washed thoroughly with water and air-dried to give dimethyl 2-(4-bromo-2-nitrophenyl)malonate A.17 in nearly quantitative yield. ¹H NMR (400 MHz, DMSO-d₆) δ 8.32 (d, J=2.0 Hz, 1H), 8.02 (dd, J=8.5, 2.0 Hz, 1H), 7.52 (d, J=8.5 Hz, 1H), 5.50 (s, 1H), 3.71 (s, 6H). LCMS-ESI (POS), M/Z, M+1: Found 331.9 and 334.0.

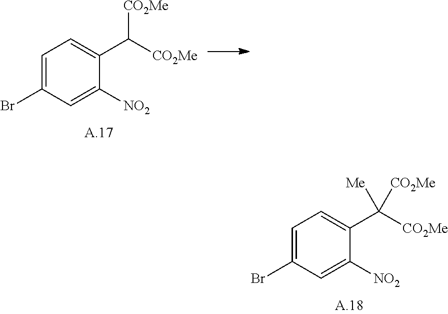

### dimethyl (4-bromo-2-nitrophenyl)(methyl)propanedioate A.18

To a solution of dimethyl 2-(4-bromo-2-nitrophenyl)malonate A.17 (70 g, 210 mmol) in 300 ml of anhydrous DMF at 0° C. was added K₂CO₃ (32 g, 231.85 mmol) under an atmosphere of nitrogen. The solution was stirred for 10 minutes before adding MeI (13.41 ml, 241.99 mmol) over a period of 0.5 hr. The solution was then allowed to slowly warm to room temperature. Stirred the solution at room temperature for 2 days before it was poured into ice cold saturated NH4Cl (1.5 L). A yellow solid was filtered off and washed with water. The yellowish solid was dissolved in ethyl acetate and transferred to a separation funnel. The organics were washed with brine and dried over MgSO4 before they were concentrated under vacuum to give dimethyl (4-bromo-2-nitrophenyl)(methyl)propanedioate A.18 (71 g, 97%) as a light brown solid.

1H NMR (500 MHz, DMSO-d₆) δ ppm 8.27 (1H, d, J=2.4 Hz), 7.96 (1H, dd, J=8.6, 2.2 Hz), 7.42 (1H, d, J=8.3 Hz), 3.66 (6H, s), 1.88 (3H, s); RPHPLC @ 8.593 min (95%).

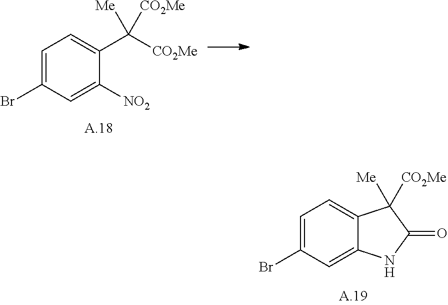

### methyl 6-bromo-3-methyl-2-oxo-2,3-dihydro-1H-indole-3-carboxylate A.19

To 450 ml of glacial acetic acid was added dimethyl (4-bromo-2-nitrophenyl)(methyl)propanedioate A.18 (71 g, 205 mmol) followed by Fe⁰ (34.35, 615 mmol). Heated the solution to 100° C. in an oil bath for 1 hour before adding more Fe⁰ (11.45 g, 205 mmol). The solution was heated at 100° C. for 3 hours before it was cooled to room temperature and concentrated under vacuum. The residue obtained was partially dissolved in ethyl acetate and a light brownish solid was filtered off and set aside. The filtrate was transferred to a separation funnel and washed with 1N HCl followed by brine. It was then dried over MgSO₄ and concentrated under vacuum. The solid obtained was triturated with ethyl acetate to give product (3 g) as a white solid. The light brownish solid from the filtration was partially dissolved in 1N HCl and stirred at room temperature overnight. It was then transferred to a separation funnel and extracted with dichloromethane. A pale white solid was filtered from the organics and washed with water to give additional product (34 g). The filtrate was dried over MgSO4 and concentrated under vacuum to give a third crop of solid (11.8 g). The combined solids were dried under vacuum to give methyl 6-bromo-3-methyl-2-oxo-2,3-dihydro-1H-indole-3-carboxylate A.19 (48.4 g, 83%) as a beige solid.

1H NMR (500 MHz, DMSO-d₆) δ ppm 10.83 (1H, s), 7.18 (2H, br s), 7.39 (1H, m), 3.58 (3H, s), 1.50 (3H, s); ESI-MS: M−282.0 m/z.

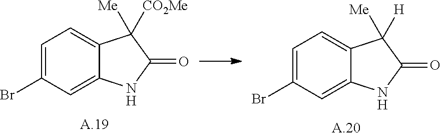

### 6-bromo-3-methyl-1,3-dihydro-2H-indol-2-one A.20

Methyl 6-bromo-3-methyl-2-oxo-2,3-dihydro-1H-indole-3-carboxylate A.19 (32.04 g, 112.8 mmol) was combined with TFA (100 ml) and 30% aqueous H₂SO₄ (30 ml). The solution was heated to 80° C. for 6 hours before being cooled to room temperature. After dilution with ice water, a precipitate was collected by filtration and dried under vacuum to give 6-bromo-3-methyl-1,3-dihydro-2H-indol-2-one A.20 (29.9 g) as a pink solid.

1H NMR (500 MHz, DMSO-d₆) δ ppm 10.44 (1H, s), 7.21 (1H, d, J=7.8 Hz), 7.12 (1H, d, J=7.8 Hz), 6.95 (1H, br. s.), 3.39 (1H, q, J=7.5 Hz), 1.31 (3H, d, J=7.8 Hz)

ESI-MS: M+H⁺226.0 m/z; RPHPLC @ 7.02 min (97%).

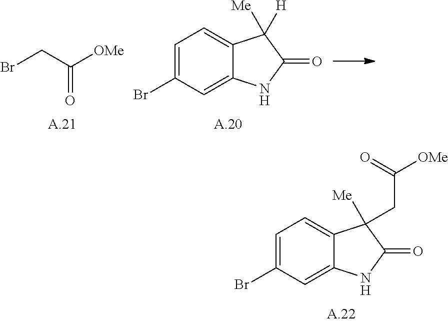

### methyl (6-bromo-3-methyl-2-oxo-2,3-dihydro-1H-indol-3-yl)acetate A.22

6-bromo-3-methyl-1,3-dihydro-2H-indol-2-one A.20 (500 mg, 2.21 mmol) and Cs₂CO₃ (1.44 g, 4.42 mmol) were combined in 4 ml of DMF. To this was then added methyl bromoacetate A.21 (203 μL, 2.21 mmol) and the solution was stirred at room temperature for 2 hours. After diluting the solution with water and adjusting the pH to 2 with 1N HCl, the aqueous layer was and extracted with ethyl acetate. The organics were then concentrated under vacuum. The residue obtained was purified by CombiFlash chromatography eluting with a gradient of dichloromethane to 3% methanol/dichloromethane. The fractions containing the product were combined and concentrated under vacuum to give methyl (6-bromo-3-methyl-2-oxo-2,3-dihydro-1H-indol-3-yl)acetate A.22 (468 mg) as an off white solid.

ESI-MS: M−296.0 m/z.

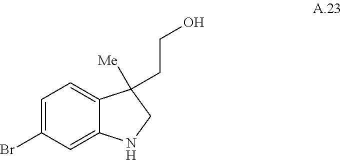

2-(6-bromo-3-methyl-2,3-dihydro-1H-indol-3-yl)ethanol A.23 was synthesized by the procedure used to prepare A.28; the title compound was obtained as a light brown gum (crude).

ESI-MS: M+256.0 m/z.

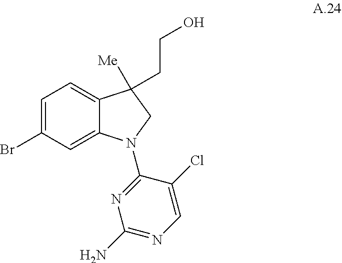

2-(6-bromo-3-methyl-1-(4-pyridinyl)-2,3-dihydro-1H-indol-3-yl)ethanol A.24 was synthesized by the procedure used to prepare A.2. The title compound was obtained as a brownish film (223 mg).ESI-MS: M+ 283.0 m/z.

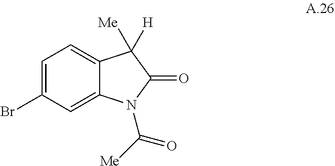

### 1-acetyl-6-bromo-3-methyl-1,3-dihydro-2H-indol-2-one A.26

6-bromo-3-methyl-1,3-dihydro-2H-indol-2-one A.20 (15 g, 66.35 mmol) was combined with 70 ml of acetic anhydride. The solution was heated at 110° C. for 2 days before it was concentrated under vacuum. The oil obtained was diluted with ethyl acetate and washed in succession with saturated NaHCO₃, water, and brine. The organics were dried over MgSO₄ before they were concentrated under vacuum. The residue obtained was heated in EtOH (80 ml) to form a clear reddish solution. After cooling to −15° C. and standing overnight, a light brown solid was collected by filtration as 1-acetyl-6-bromo-3-methyl-1,3-dihydro-2H-indol-2-one A.26 (8.8 g, 50%).

1H NMR (500 MHz, DMSO-d₆) δ ppm 8.25 (1H, d, J=1.5 Hz), 7.43 (1H, dd, J=7.8, 1.4 Hz), 7.38 (1H, d, J=8.3 Hz), 3.78 (7H, a, J=7.3 Hz), 2.56 (3H, s), 1.42 (3H, d, J=7.8 Hz).

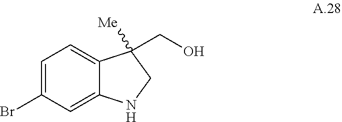

### (6-bromo-3-methyl-2,3-dihydro-1H-indol-3-yl)methanol A.28

Under an atmosphere of N₂ a solution of methyl 6-bromo-3-methyl-2-oxo-2,3-dihydro-1H-indole-3-carboxylate A.19 (5.0 g, 17.5 mmol) in anhydrous toluene (50 ml), and was heated to 85° C. A solution of RedAl (3.4M in toluene)(15 ml, 51 mmol) was then cautiously added dropwise over a period of 25 minutes. Followed by the addition of more RedAl (3.4M in toluene) (10 ml, 34 mmol) over a period of 3 min. At 45 minutes the solution was cooled to 0° C. The solution was quenched with the slow addition of 1N NaOH and then allowed to slowly warm to room temperature overnight. The next day the solution was filtered through a pad of celite and washed with ethyl acetate. The filtrate was washed with brine and then dried over MgSO₄. After concentrating the organics under vacuum the brown oil obtained was purified by flash chromatography eluting with 1.5% methanol/dichloromethane. The fractions containing the product were combined and concentrated under vacuum to give (6-bromo-3-methyl-2,3-dihydro-1H-indol-3-yl)methanol A.28 (1.6 g, 44%) as a clear light brown oil.

1H NMR (500 MHz, DMSO-d₆) δ ppm 6.86 (1H, d, J=7.8 Hz), 6.61 (1H, dd, J=7.6, 1.7 Hz), 6.56 (1H, d, J=2.0 Hz), 5.71 (1H, s), 4.81 (1H, t, J=5.6 Hz), 3.44 (1H, d, J=7.8 Hz), 3.31-3.35 (1H, m), 3.24-3.29 (1H, m), 3.05 (1H, dd, J=8.8, 2.0 Hz), 1.19 (3H, s); ESI-MS: M+H⁺ 242.4 m/z.

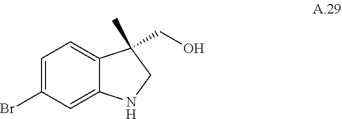

((3S*)-6-bromo-3-methyl-2,3-dihydro-1H-indol-3-yl)methanol (A.29). This compound was resolved by chiral HPLC eluting as the second peak using an AD-H column with isocratic 15% isopropanol in hexane. [α]^{D}₂₃=+29 (c=0.49 in methanol). Stereochemistry is arbitrarily assigned.

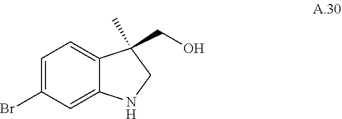

((3R*)-6-bromo-3-methyl-2,3-dihydro-1H-indol-3-yl)methanol (A.30). This compound was resolved by chiral HPLC eluting as the first peak using an AD-H column with isocratic 15% isopropanol in hexane. [α]^{D}₂₃=−31.8 (c=0.49 in methanol). Stereochemistry is arbitrarily assigned.

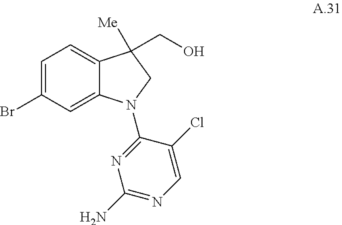

(1-(2-amino-5-chloro-4-pyrimidinyl)-6-bromo-3-methyl-2,3-dihydro-1H-indol-3-yl)methanol A.31 was synthesized from A.28 by the procedure used to prepare A.2; the title compound was obtained as an off white solid (193 mg, 32%).

ESI-MS: M+H⁺ 269.0 m/z.

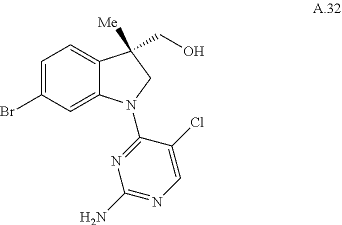

((3S*)-1-(2-amino-5-chloro-4-pyrimidinyl)-6-bromo-3-methyl-2,3-dihydro-1H-indol-3-yl)methanol A.32 was synthesized from A.29 by the procedure used to prepare A.2; the title compound was obtained as an orange solid (183 mg). Stereochemistry is arbitrarily assigned.

ESI-MS: M+H⁺ 269.0 m/z.

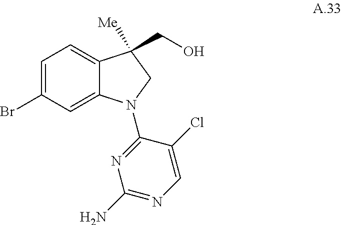

((3R*)-1-(2-amino-5-chloro-4-pyrimidinyl)-6-bromo-3-methyl-2,3-dihydro-1H-indol-3-yl)methanol A.33 was synthesized from A.30 by the procedure used to prepare A.2; the title compound was obtained as an orange solid (220 mg). Stereochemistry is arbitrarily assigned.

ESI-MS: M+H⁺ 269.0 m/z.

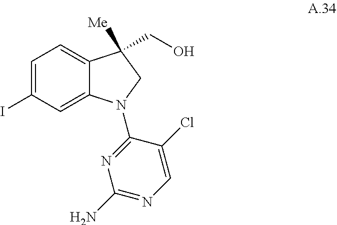

### ((3S*)-1-(2-amino-5-chloro-4-pyrimidinyl)-6-iodo-3-methyl-2,3-dihydro-1H-indol-3-yl)methanol A.34

((3S*)-1-(2-amino-5-chloro-4-pyrimidinyl)-6-bromo-3-methyl-2,3-dihydro-1H-indol-3-yl)methanol A.32 (90 mg, 0.243 mmol), CuI (3 mg, 0.016 mmol), NaI (72 mg, 486 mmol), and N,N′-dimethyl-1,2-ethanediamine (30 μL, 0.281 mmol) were combined in 6 ml of n-BuOH under an atmosphere of nitrogen. The solution was heated to a reflux for 5 hours before adding more CuI (28 mg, 0.147 mmol), and NaI (172 mg, 1.15 mmol). 2 days later added more NaI (72 mg, 486 mmol) and continued to reflux overnight before it was cooled to room temperature. Diluted the solution with water and extracted with ethyl acetate. The organics were then washed with Brine and dried over MgSO4 before being concentrated under vacuum. The residue obtained was purified by CombiFlash chromatography eluting with a gradient of 1% methanol/dichloromethane to 5% methanol/dichloromethane. The fractions containing the product were combined and concentrated under vacuum to give (1-(2-amino-5-chloro-4-pyrimidinyl)-6-iodo-3-methyl-2,3-dihydro-1H-indol-3-yl)methanol A.34 (138 mg) as a clear yellowish film.

ESI-MS: M+H⁺ 417.0 m/z, Stereochemistry is arbitrarily assigned.

### 6-bromoindolin-2-one A.35

Ref. Quallich, G. J.; Morrissey, P. M. Synthesis 1993, 51-53.

To a stirred solution of methyl (4-bromo-2-nitrophenyl)acetate A.97 (6.5 g, 23.7 mmol) in glacial acetic acid (80 mL) was added iron powder (6.6 g, 5 eq.) at room temperature. The resulting mixture was stirred in an oil bath preheated at 100° C. for 2 h at which time LC-MS showed completion. The mixture was filtered, while still warm, through a layer of celite and more glacial acetic acid was used to wash off the residual product. The filtrate was concentrated under high vacuum. The residue was mixed with ice and saturated NaHCO₃ aqueous solution and extracted with ethyl acetate (2×). The combined organics were washed with saturated NaHCO₃ aqueous solution (1×), brine (2×) and dried over Na₂SO₄. The residue after concentration in vacuo was triturated with ethyl acetate/hexanes to give 6-bromoindolin-2-one A.35 (4.8 g, 95%) as an off-white solid. ¹H NMR (400 MHz, DMSO-d₆) δ 10.48 (br s, 1H), 7.16 (d, J=6.4 Hz, 1H), 7.11 (dd, J=6.4, 1.2 Hz, 1H), 6.95 (d, J=1.2 Hz, 1H), 3.45 (s, 2H). LCMS-ESI (POS), M/Z, M+1: Found 212.0 and 214.0.

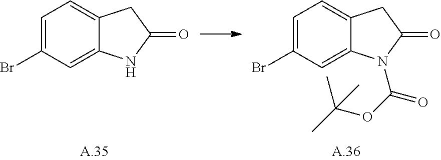

### tert-butyl 6-bromo-2-oxo-2,3-dihydro-1H-indole-1-carboxylate A.36

(By analogy to Akai, S., T. Tsujino, et al. (2004). J. Org. Chem. 69(7): 2478-2486.) To a stirred mixture of 6-bromoindolin-2-one A.35 (5.0 g, 23.6 mmol) and NaHCO₃ (10 eq., 21.0 g) in THF (130 mL) was added (Boc)₂O (2.5 eq., 14.0 g) at room temperature under N₂. The resulting mixture was heated at reflux for 3 h. After cooling, the mixture was vacuum filtered through a layer of celite and the filter cake was thoroughly washed with THF. The filtrate was concentrated in vacuo and the residue was subjected to combi-flash column chromatography (ethyl acetate/hexanes) to give tert-butyl 6-bromo-2-oxo-2,3-dihydro-1H-indole-1-carboxylate A.36 (6.0 g, 81% yield) as an off-white solid. ¹H NMR (400 MHz, CHLOROFORM-d) δ ppm 8.04 (1H, d, J=1.6 Hz), 7.29 (1H, dd, J=8.0, 1.8 Hz), 7.11 (1H, d, J=7.8 Hz), 3.60 (2H, s), 1.65 (9H, s). LCMS-ESI (POS), M/Z, M+Na⁺: Found 334.0 and 336.0.

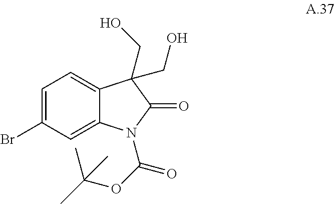

### tert-butyl 6-bromo-3,3-bis(hydroxymethyl)-2-oxo-2,3-dihydro-1H-indole-1-carboxylate A.37

(By analogy to Akai Koichi Nakazawa, Masaki Hayashi, et al. (2001) Tetrahedron: Asymmetry 12(6): 897-901.) To a stirred mixture of tert-butyl 6-bromo-2-oxo-2,3-dihydro-1H-indole-1-carboxylate (1.8 g, 5.77 mmol) and K₂CO₃ (3.0 eq., 2.4 g) in THF (100 mL) was added paraformaldehyde (24.0 eq., 4.4 g) at room temperature. The resulting mixture was stirred at ambient temperature for 1 h. Upon workup, the mixture was poured into ice and saturated NaHCO₃ aqueous solution and extracted with ethyl acetate (2×). The combined organics were washed with brine (1×), dried over Na₂SO₄, and concentrated in vacuo. The residue was subjected to combi-flash column chromatography (methanol/dichloromethane) to give tert-butyl 6-bromo-3,3-bis(hydroxymethyl)-2-oxo-2,3-dihydro-1H-indole-1-carboxylate A.37 (1.87 g, 87% yield) as a white solid. ¹H NMR (400 MHz, DMSO-d₆) δ ppm 7.89 (1H, s), 7.41 (2H, d, J=0.8 Hz), 4.96 (2H, t, J=5.1 Hz), 3.62-3.76 (4H, m), 1.57 (9H, s). LCMS-ESI (POS), M/Z, M+Na+: Found 394.0 and 396.0.

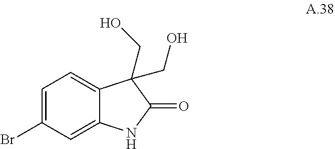

### 6-bromo-3,3-bis(hydroxymethyl)-1,3-dihydro-2H-indol-2-one A.38

tert-butyl 6-bromo-3,3-bis(hydroxymethyl)-2-oxo-2,3-dihydro-1H-indole-1-carboxylate A.37 (2.98 g, 8.00 mmol) was combined with 70 ml of dichloromethane. To this was then added TFA (6 ml) and the reaction was stirred at room temperature for 1 hour. Diluted the solution with dichloromethane and washed the organics with Brine. The aqueous washings were then extracted with ethyl acetate. The organics were combined and dried over Na₂SO₄ before they were concentrated under vacuum to give 6-bromo-3,3-bis(hydroxymethyl)-1,3-dihydro-2H-indol-2-one A.38 (2.44 g) as a white gum.

ESI-MS: M−270.0 m/z.

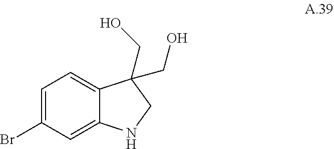

### (6-bromo-2,3-dihydro-1H-indole-3,3-diyl)dimethanol A.39

Under an atmosphere of nitrogen, 6-bromo-3,3-bis(hydroxymethyl)-1,3-dihydro-2H-indol-2-one A.38 (2.30 g, 8.45 mmol) was dissolved in 5 ml of anhydrous THF. The solution was cooled in an ice bath before adding BH₃ (10.1M in Me₂S , 4.2 ml, 42.26 mmol) over a period of 20 min. The reaction was then stirred overnight at room temperature. The solution was diluted with 10 ml of THF and quenched with the slow addition of ice followed by the slow addition of concentrated HCl (3 ml). After warming the solution to room temperature it was stirred for 15 min and then concentrated under vacuum. The solution was diluted with methanol and concentrated under vacuum, repeated four times to provide (6-bromo-2,3-dihydro-1H-indole-3,3-diyl)dimethanol A.39 (1.9 g,) as a white solid. ESI-MS: M+H⁺ 258.0 m/z.

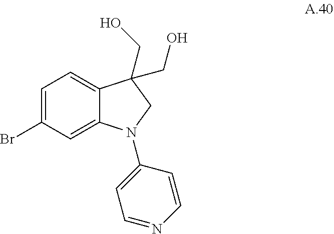

### (6-bromo-1-(4-pyridinyl)-2,3-dihydro-1H-indole-3,3-diyl)dimethanol A.40

Under an atmosphere of nitrogen, 4-chloropyridine hydrochloride (A.216) (58 mg, 0.387 mmol) and (6-bromo-2,3-dihydro-1H-indole-3,3-diyl)dimethanol A.39 (100 mg, 0.387) were combined in 1.5 ml of 1-pentanol and then heated to 140° C. for 1.5 hours. The solution was cooled to room temperature, diluted with saturated NaHCO₃, and extracted with ethyl acetate. The organics were dried over Na₂SO₄ before being concentrated under vacuum. The residue obtained was purified by CombiFlash chromatography eluting with a gradient of 4% methanol/0.4% triethylamine/dichloromethane to 10% methanol/0.9% triethylamine/dichloromethane. The fractions containing the product were combined and concentrated under vacuum to give (6-bromo-1-(4-pyridinyl)-2,3-dihydro-1H-indole-3,3-diyl)dimethanol A.40 (100 mg) as a white solid.

ESI-MS: M+H⁺ 335.0 m/z.

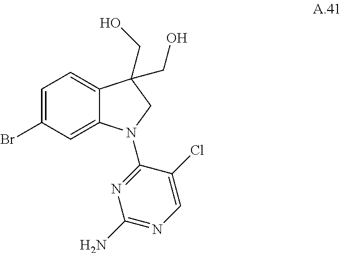

(1-(2-amino-5-chloro-4-pyrimidinyl)-6-bromo-2,3-dihydro-1H-indole-3,3-diyl)dimethanol A.41 was synthesized by the procedure used to prepare compound A.2. The title compound was obtained as a brownish film (1.8 g).

ESI-MS: M+H⁺ 385.0 m/z.

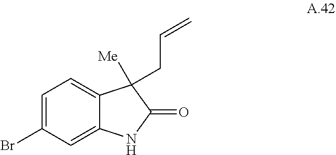

6-bromo-3-methyl-3-(2-propen-1-yl)-1,3-dihydro-2H-indol-2-one A.42 was synthesized from A.20 and allyl bromide by the procedure used to prepare A.22; the title compound was obtained as a light brown solid (300 mg).

1H NMR (500 MHz, DMSO-d₆) δ ppm 10.47 (1H, s), 7.22 (1H, d, J=7.8 Hz), 7.14 (1H, dd, J=7.8 Hz, 1.4 Hz), 6.95 (1H, d=1.9 Hz), 5.29-5.43 (1H, m), 4.84-4.99 (2H, m), 2.45-2.53 (1H, m), 2.33-2.42 (1H, m, J=13.4, 7.6 Hz), 1.24 (3H, s); ESI-MS: M−264.0 m/z.

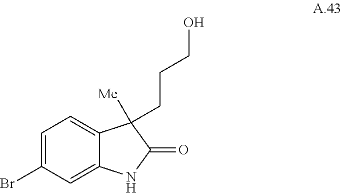

### 6-bromo-3-(3-hydroxypropyl)-3-methyl-1,3-dihydro-2H-indol-2-one A.43

6-Bromo-3-methyl-3-(2-propen-1-yl)-1,3-dihydro-2H-indol-2-one A.42 (300 mg, 1.13 mmol) was dissolved in 2 ml of anhydrous THF under an atmosphere of nitrogen cooled in an ice bath. To this was added slowly 20 ml of 9-BBN (0.5M in THF). The solution was allowed to warm to room temperature and stirred overnight. The next day the solution was cooled in an ice bath before slowly adding a solution of 2.25 ml of 50% H₂O₂ and 25 ml of 3N NaOH. After warming the solution to room temperature it was stirred for 3 hours. Diluted the solution with saturated NaHCO3 and extracted with ethyl acetate. The combined organics were washed with Brine and dried over Na₂SO₄ before being concentrated under vacuum. The residue obtained was purified by CombiFlash chromatography eluting with a gradient of 2.5% methanol/dichloromethane to 5% methanol/dichloromethane. The fractions containing the product were combined and concentrated under vacuum to give 6-bromo-3-(3-hydroxypropyl)-3-methyl-1,3-dihydro-2H-indol-2-one A.43 (271 mg) as a light brown film.

ESI-MS: M+H⁺ 284.0 m/z.

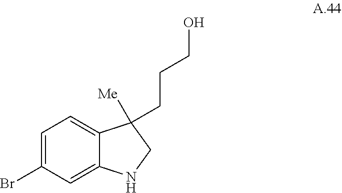

3-(6-bromo-3-methyl-2,3-dihydro-1H-indol-3-yl)-1-propanol A.44 was synthesized by the procedure used to prepare compound A.28; the title compound was obtained as a clear oil (62 mg).

ESI-MS: M+H⁺ 270.1 m/z.

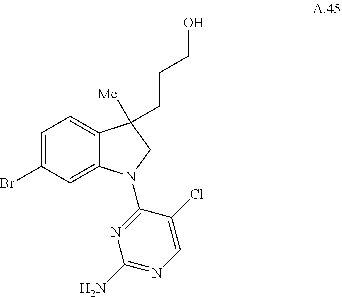

3-(1-(2-amino-5-chloro-4-pyrimidinyl)-6-bromo-3-methyl-2,3-dihydro-1H-indol-3-yl)-1-propanol A.45 was synthesized by the procedure used to prepare compound A.2; the title compound was obtained as a tan colored solid (114 mg).

ESI-MS: M+H⁺ 397.1 m/z.

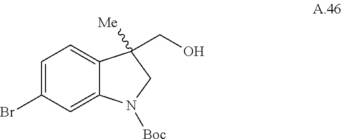

### tert-butyl 6-bromo-3-(hydroxymethyl)-3-methyl-2,3-dihydro-1H-indole-1-carboxylate A.46

To a solution of (6-bromo-3-methyl-2,3-dihydro-1H-indol-3-yl)methanol A.28 (1.55 g, 6.40 mmol), and DIEA (1.23 ml, 7.24 mmol), in 30 ml of dichloromethane was added Boc-anhydride (1.52 g, 6.96 mmol) at 0° C. The solution was then stirred overnight at room temperature before it was diluted with dichloromethane and washed with sat. NaHCO₃. The organics were dried over MgSO₄ before being concentrated under vacuum. Obtained tert-butyl 6-bromo-3-(hydroxymethyl)-3-methyl-2,3-dihydro-1H-indole-1-carboxylate A.46 (2.04 g) as a clear oil.

¹H NMR (500 MHz, DMSO-d₆) δ 7.83 (br s, 1H), 7.141 (d, J=6.4 Hz, 1H), 7.11 (dd, J=6.0, 0.8 Hz, 1H), 4.98 (t, J=4.4 Hz, 1H), 3.94 (d, J=8.8, 1H), 3.50 (d, J=8.8, 1H), 3.36 (dd, J=8.4, 4.4 Hz, 1H), 3.33 (dd, J=8.8, 4.4 Hz, 1H), 1.49 (s, 9H), 1.22 (s, 3H); ESI-MS: 2M+Na⁺ 707.0 m/z.

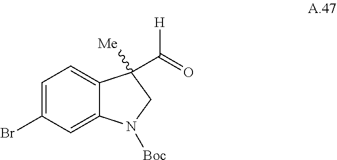

tert-butyl 6-bromo-3-formyl-3-methyl-2,3-dihydro-1H-indole-1-carboxylate A.47 tert-butyl 6-bromo-3-(hydroxymethyl)-3-methyl-2,3-dihydro-1H-indole-1-carboxylate A.46 (5.23 g, 15.28 mmol) was dissolved in 25 ml of dichloromethane and 25 ml of acetonitrile. To this was added Dess-Martin Periodinane (16.9, 39.73 mmol) and the solution was stirred at room temperature overnight. The next day the solution was concentrated under vacuum. The residue obtained was diluted with ether, filtered through a pad of celite, and washed with ether. The filtrate was partitioned with saturated NaHCO₃ and the aqueous layer was washed with ethyl acetate. The combined organics were dried over Na₂SO₄ before being concentrated under vacuum. The residue obtained was purified by CombiFlash chromatography eluting with 40% hexane/60% dichloromethane. The fractions containing the product were combined and concentrated under vacuum to give tert-butyl 6-bromo-3-formyl-3-methyl-2,3-dihydro-1H-indole-1-carboxylate A.47 (2.50 g, 48%) as a clear oil.

1H NMR (500 MHz, DMSO-d₆) δ ppm 9.56 (1H, s), 7.23 (1H, d, J=7.8 Hz), 7.20 (1H, dd, J=7.8, 1.9 Hz), 4.45 (1H, d, J=11.2 Hz), 3.70 (1H, d, J=11.7 Hz), 1.50 (9H, br. s.), 1.45 (3H, s).

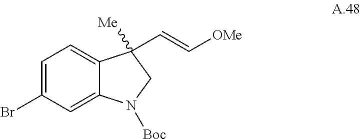

### tert-butyl 6-bromo-3-((E)-2-methoxyethenyl)-3-methyl-2,3-dihydro-1H-indole-1-carboxylate A.48

To potassium tert butoxide (0.440 g, 3.92 mmol) in 5 ml of anhydrous THF under an atmosphere of N₂ was added (methoxymethyl)triphenylphosphine chloride (1.22 g, 3.36 mmol) slowly. After 1.5 hours of stirring the solution was cooled in an ice bath before adding tert-butyl 6-bromo-3-formyl-3-methyl-2,3-dihydro-1H-indole-1-carboxylate A.47 (0.970 g, 2.05 mmol) as a solution dissolved in 4 ml of anhydrous THF. The solution was then warmed to room temperature and stirred for 2 hours before it was diluted with water and extracted with dichloromethane. The organics were dried over MgSO₄ before being concentrated under vacuum. The residue obtained was purified by CombiFlash chromatography eluting with a gradient of hexane to dichloromethane. The fractions containing the product were combined and concentrated under vacuum to give tert-butyl 6-bromo-3-((E)-2-methoxyethenyl)-3-methyl-2,3-dihydro-1H-indole-1-carboxylate (0.600 g, 57%) as a light brown oil.

1H NMR (500 MHz, DMSO-d₆) δ ppm 7.12-7.16 (2H, m), 7.06 (1H, d, J=7.8 Hz), 6.30 (1 H, d, J=12.7 Hz), 4.98 (1H, d, J=13.2 Hz), 3.81 (1H, d, J=10.8 Hz), 3.72 (1H, d, J=10.8 Hz), 3.44 (3H, s), 1.51 (9H, s), 1.33 (3H, s). See a 2:1 ratio of E:Z isomers.

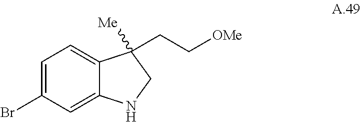

### 6-bromo-3-(2-methoxyethyl)-3-methyl-2,3-dihydro-1H-indole A.49

tert-butyl 6-bromo-3-((E)-2-methoxyethenyl)-3-methyl-2,3-dihydro-1H-indole-1-carboxylate A.48 (483 mg, 1.31 mmol) and NaBH(OAc)₃ (3.32 g, 15.74 mmol) were combined in 10 ml of dichloromethane. To this was added 1 ml of TFA and the solution was stirred at room temperature over the weekend. The reaction was then quenched with 1N NaOH and extracted with ethyl acetate. The organic layer was dried over MgSO₄ and then concentrated under vacuum. The residue obtained was dissolved in 2 ml of TFA and stirred at room temperature for 2 hours before it was concentrated under vacuum. The residue obtained was purified by CombiFlash chromatography eluting with dichloromethane to 2% methanol/dichloromethane. The fractions containing the product were combined and concentrated under vacuum to give 6-bromo-3-(2-methoxyethyl)-3-methyl-2,3-dihydro-1H-indole A.49 (121 mg) as a brownish film.

ESI-MS: M+H⁺ 270.0 m/z.

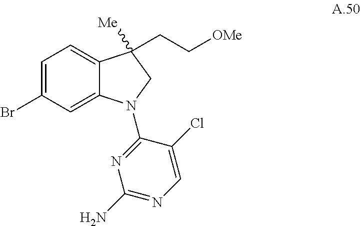

4-(6-bromo-3-(2-methoxyethyl)-3-methyl-2,3-dihydro-1H-indol-1-yl)-5-chloro-2-pyrimidinamine A.50 was synthesized from A.49 and A.14 by the procedure used to prepare A.2; the title compound was obtained as a yellowish film (20 mg, 25%).

ESI-MS: M+H⁺ 397.0 m/z.

### sodium ethyl (2E)-2-fluoro-3-hydroxy-2-propenoate A.51

To 140 ml of anhydrous ether was added in 1 g portions NaH (60% im mineral oil) (6.76 g, 169 mmol) slowly at 0° C. under an atmosphere of nitrogen. Absolute EtOH (0.8 ml) was then added to the reaction, followed by the dropwise addition of a solution of ethylformate (13.65 ml, 169 mmol) and ethylfluoroacetate (16.3 ml, 169 mmol) in 100 ml of ether over a period of 1.5 hr. The addition funnel was rinsed with 20 ml of ether and the solution was stirred for 19 hours at room temperature. After concentrating the solution under vacuum a pale yellow solid was obtained and carried on to the next step.

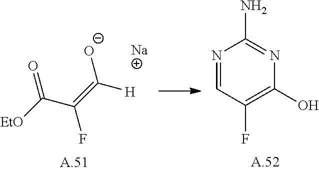

### 2-amino-5-fluoro-4-pyrimidinol A.52

[Biressi, e. a. (1963). Gazz. Chim. Ital. 93: 1268]

Guanidine HCl salt (49.5 g, 518 mmol) was neutralized by addition to a solution of NaOEt (2.68M in EtOH, 193 ml) cooled in an ice bath. The solution was stirred for 30 minutes before filtering off the sodium chloride generated through a Buchner funnel. The filtrate was then added to sodium ethyl (2E)-2-fluoro-3-hydroxy-2-propenoate A.51 in 177 ml of EtOH. The solution was heated to 90° C. for 18 hours and then it was concentrated under vacuum. To the residue obtained was added 200 ml of water followed by concentrated HCl dropwise until a pH of 5 was reached. The solids were collected by filtration through a Buchner funnel and washed with 50 ml of water to give 56.46 g of a brownish solid. 46 g of this material was purified by flash chromatography eluting with 5% water/acetonitrile. The fractions containing the product were combined and concentrated under vacuum to give 2-amino-5-fluoro-4-pyrimidinol A.52 (12.53 g) as a light brown solid.

1H NMR (500 MHz, DMSO-d₆) δ ppm 11.49 (1H, br. s.), 7.62 (1H, d, J=3.9 Hz), 6.54 (2H, br. s.)

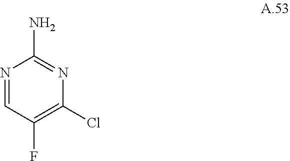

4-chloro-5-fluoro-2-pyrimidinamine A.53 was synthesized by the procedure used to prepare A.12; the title compound was obtained as a brown solid (8.66 g).

1H NMR (500 MHz, DMSO-d₆) δ ppm 8.38 (1H, d, J=1.5 Hz), 7.06 (2H, br. s.)

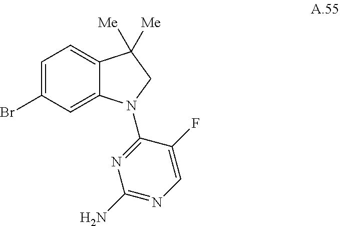

4-(6-bromo-3,3-dimethyl-2,3-dihydro-1H-indol-1-yl)-5-fluoro-2-pyrimidinamine A.55 was synthesized from A.53 and 6-bromo-3,3-dimethylindoline A.54 [Atwal, K. S., F. N. Ferrara, et al. (1996) J. Med. Chem. 39(1): 304-13] by the procedure used to prepare A.2; the title compound was obtained as a brown solid (500 mg). ESI-MS: M+H⁺337.0 m/z

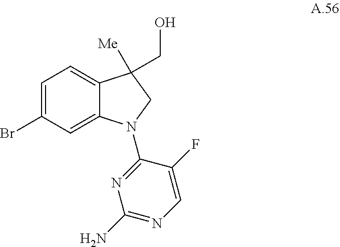

(1-(2-amino-5-fluoro-4-pyrimidinyl)-6-bromo-3-methyl-2,3-dihydro-1H-indol-3-yl)methanol A.56 was synthesized from A.28 and A.53 by the procedure used to prepare A.2; the title compound was obtained as a off white solid (129 mg, 20%).

ESI-MS: M+H⁺ 353.0 m/z

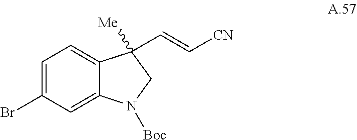

tert-butyl 6-bromo-3-((E/Z)-2-cyanoethenyl)-3-methyl-2,3-dihydro-1H-indole-1-carboxylate A.57 was synthesized from A.47 by the procedure used to prepare A.48 and carried on to the next reaction.

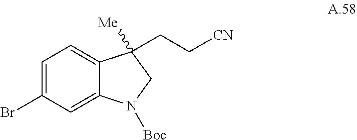

tert-butyl 6-bromo-3-(2-cyanoethyl)-3-methyl-2,3-dihydro-1H-indole-1-carboxylate A.58 tert-butyl 6-bromo-3-((E/Z)-2-cyanoethenyl)-3-methyl-2,3-dihydro-1H-indole-1-carboxylate A.57 (290 mg, 0.78 mmol), was combined with NaBH₄ (50 mg, 1.32 mmol) in 10 ml of dimethoxyethane and stirred at room temperature for 1 hour before being heated to 70° C. for 2 hours. The solution was cooled to room temperature and then quenched with saturated NH₄Cl. methanol was added to the solution and it was then concentrated under vacuum. The methanol dilution and concentration sequence was repeated 3 times. The residue obtained was diluted with water and extracted with ether. The organics were dried over MgSO₄ before they were concentrated under vacuum. The residue obtained was purified by CombiFlash chromatography. The fractions containing the product were combined and concentrated under vacuum to give tert-butyl 6-bromo-3-(2-cyanoethyl)-3-methyl-2,3-dihydro-1H-indole-1-carboxylate A.58 (228 mg, 78%) as a clear oil.

1H NMR (500 MHz, DMSO-d₆) δ ppm 7.84 (1H, br. s.), 7.21 (1H, d, J=), 7.15 (1H, dd, J=7.8, 2.0 Hz), 3.89 (1H, d, J=11.7 Hz), 3.60 (1H, d, J=11.2 Hz), 2.36-2.45 (1H, m), 2.19-2.30 (1H, m), 1.86-2.01 (2H, m), 1.50 (9H, s), 1.27 (3H, s).

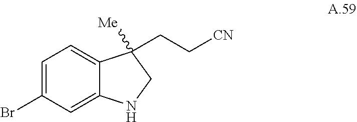

3-(6-bromo-3-methyl-2,3-dihydro-1H-indol-3-yl)propanenitrile A.59 was synthesized by the procedure used to prepare A.38 and carried on to the next step.

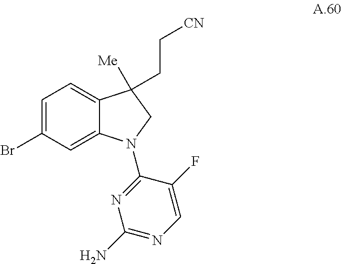

3-(1-(2-amino-5-fluoro-4-pyrimidinyl)-6-bromo-3-methyl-2,3-dihydro-1H-indol-3-yl)propanenitrile A.60 was synthesized by the procedure used to prepare A.2; the title compound was obtained as a brownish solid (137 mg, 58%).

1H NMR (500 MHz, DMSO-d₆) δ ppm 8.41 (1H, s), 8.22 (1H, d, J=7.3 Hz), 7.51 (2H, br. s.), 7.29-7.32 (2H, m), 4.32 (1H, dd, J=11.2, 5.4 Hz), 4.04 (1H, dd, J=11.5, 4.6 Hz), 2.43-2.49 (1H, m), 2.23-2.32 (1H, m), 1.90-2.04 (2H, m), 1.34 (3H, s)

ESI-MS: M+H⁺ 376.0 m/z.

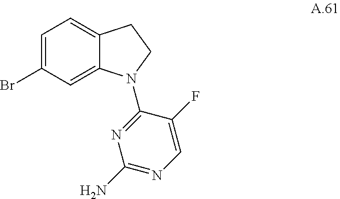

4-(6-bromo-2,3-dihydro-1H-indol-1-yl)-5-fluoro-2-pyrimidinamine A.61 was synthesized from A.1 and A.53 by the procedure used to prepare A.2; the title compound was obtained as a brown solid (273 mg).

ESI-MS: M+H⁺ 309.0 m/z

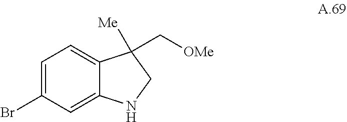

### 6-bromo-3-(methoxymethyl)-3-methyl-2,3-dihydro-1H-indole A.69

tert-butyl 6-bromo-3-(hydroxymethyl)-3-methyl-2,3-dihydro-1H-indole-1-carboxylate A.46 (450 mg, 1.31 mmol) was dissolved in 10 ml of DMF under an atmosphere of nitrogen. To this was added NaH (60% in mineral oil) (78 mg, 1.97 mmol) and the solution was stirred at room temperature for 10 minutes. MeI (0.085 ml, 1.37 mmol) was then added and the solution was stirred for 3 hours. The solution was diluted with ice water extracted with ether. The organics were dried over MgSO₄ and then concentrated under vacuum. The residue obtained was purified by CombiFlash chromatography eluting with a gradient of dichloromethane to 1% methanol/dichloromethane. The fractions containing the product were combined and concentrated under vacuum. The residue obtained was dissolved in dichloromethane (3 ml) and TFA (1.5 ml) was added. The solution was stirred at room temperature overnight. The next day the solution was concentrated under vacuum to give 6-bromo-3-(methoxymethyl)-3-methyl-2,3-dihydro-1H-indole (170 mg, 45%) A.69.

ESI-MS: M+H⁺256.0 m/z

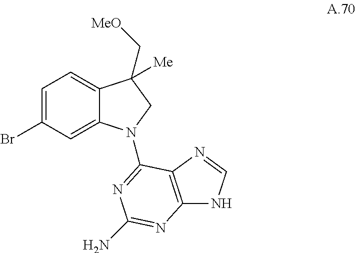

### 6-(6-bromo-3-(methoxymethyl)-3-methyl-2,3-dihydro-1H-indol-1-yl)-9H-purin-2-amine A.70

6-bromo-3-(methoxymethyl)-3-methyl-2,3-dihydro-1H-indole A.69 (170 mg, 0.60 mmol) was combined with 2-amino-6-chloropurine (200 mg, 1.2 mmol) in water (20 ml). The pH was adjusted to 2 with 1N HCl and then the solution was heated to 70° C. for 5 hours. The solution was cooled to room temperature and diluted with saturated NaHCO₃ and then extracted with ethyl acetate. The organics were dried over MgSO4 and then concentrated under vacuum. The residue obtained was purified by CombiFlash chromatography eluting with 5% methanol/dichloromethane. The fractions containing the product were combined and concentrated under vacuum to give 6-(6-bromo-3-(methoxymethyl)-3-methyl-2,3-dihydro-1H-indol-1-yl)-9H-purin-2-amine (40 mg, 17%) as a white solid.

ESI-MS: M+H⁺ 389.0 m/z

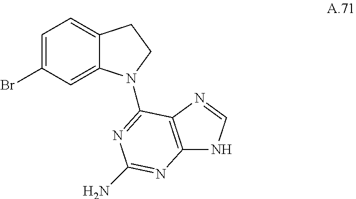

6-(6-bromo-2,3-dihydro-1H-indol-1-yl)-9H-purin-2-amine A.71 was synthesized from A.1 by the procedure used to prepare A.70; the title compound was obtained as a white solid (538 mg, 66%).

ESI-MS: M+H⁺ 331.3 m/z.

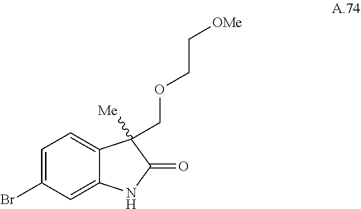

### 6-bromo-3-((2-methoxyethoxy)methyl)-3-methyl-1,3-dihydro-2H-indol-2-one A.74

Under an atmosphere of nitrogen 1-acetyl-6-bromo-3-methyl-1,3-dihydro-2H-indol-2-one A.26 (700 mg, 2.61 mmol) was dissolved in 4 ml of anhydrous THF at 0° C. To this was cautiously added NaH (60% in mineral oil) (110 mg, 2.74 mmol) followed by MEMCl (0.596 ml, 5.22 mmol). The reaction was stirred at room temperature for 2 hours. 10 ml of EtOH was added followed by 10 ml of 1N NaOH and the solution was stirred overnight. The next day the solution was diluted with saturated NH₄Cl and extracted with ethyl acetate. The organics were combined and washed with Brine before being dried over MgSO₄ and concentrated under vacuum. The residue obtained was purified by CombiFlash chromatography eluting with 20% ethyl acetate/dichloromethane. The fractions containing the product were combined and concentrated under vacuum to give 6-bromo-3-((2-methoxyethoxy)methyl)-3-methyl-1,3-dihydro-2H-indol-2-one (380 mg) as a white solid.

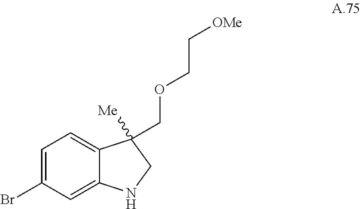

6-bromo-3-((2-methoxyethoxy)methyl)-3-methyl-2,3-dihydro-1H-indole A.75 was synthesized from A.74 by the procedure used to prepare A.28; the title compound was obtained as a light brownish oil (322 mg).

ESI-MS: M+H⁺ 300.4 m/z.

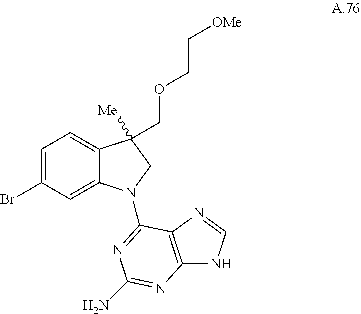

6-(6-bromo-3-((2-methoxyethoxy)methyl)-3-methyl-2,3-dihydro-1H-indol-1-yl)-9H-purin-2-amine A.76 was synthesized from A.75 by the procedure used to prepare A.70; the title compound was obtained as an off white solid (41 mg, 17%).

ESI-MS: M+ H⁺ 433.3 m/z

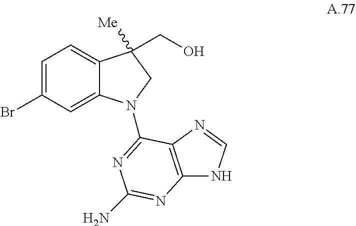

(1-(2-amino-9H-purin-6-yl)-6-bromo-3-methyl-2,3-dihydro-1H-indol-3-yl)methanol A.77 was synthesized from A.28 by the procedure used to prepare A.70; the title compound was obtained as an off white solid (40 mg, 17%).

ESI-MS: M+ H⁺ 3757.1 m/z.

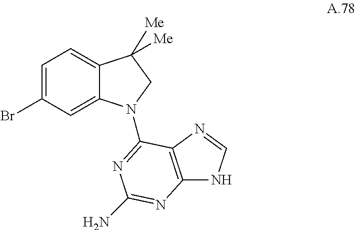

6-(6-bromo-3,3-dimethyl-2,3-dihydro-1H-indol-1-yl)-9H-purin-2-amine A.78 was obtained as a white solid (170 mg, 40%) from 6-bromo-3,3-dimethylindoline A.54 by the procedure used to prepare A.70.

ESI-MS: M+ H⁺ 359.0 m/z.

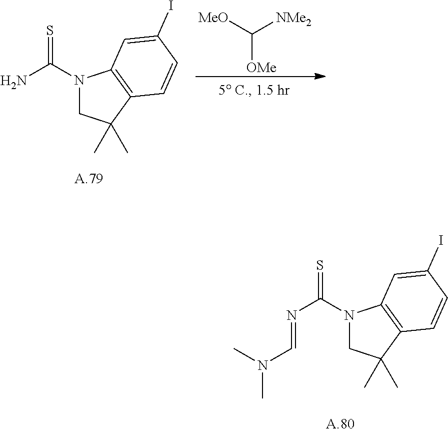

### 6-iodo-3,3-dimethylindoline-1-carbothioamide A.79

A.79 was prepared from A.10 in analogy to the procedure of Zhang, M., R. Dally, et al. (2004). Syn. Comm. 34(21): 4023-4030.

### N-((dimethylamino)methylene)-6-iodo-3,3-dimethylindoline-1-carbothioamide A.80

Dimethylformamide dimethoxyacetal (14.3 mL, 120 mmol) was cooled to 5° C. 6-iodo-3,3-dimethylindoline-1-carbothioamide A.79 (2.66 g, 8 mmol) was added slowly over 5 minutes, and the reaction stirred at 5° C. for 1.5 hours. Concentration under vacuum afforded A.80 3.1 g as an oil. 1H NMR (500 MHz, DMSO-d₆) δ ppm 8.80 (1H, s), 8.76 (1H, s), 7.37 (1H, d, J=7.6 Hz), 7.11 (1H, d, J=7.8 Hz), 4.16 (2H, s), 3.27 (3H, s), 3.16 (3H, s), 1.28 (6H, s)

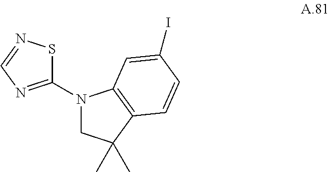

### 6-iodo-3,3-dimethyl-1-(1,2,4-thiadiazol-5-yl)indoline A.81

A solution of N-((dimethylamino)methylene)-6-iodo-3,3-dimethylindoline-1-carbothioamide A.80 (3.1 g, 8 mmol), pyridine (1.31 mL, 16 mmol) and 20 mL EtOH was stirred at room temp. A solution of hydroxylaminesulfonic acid (0.905 g, 8 mmol) in 10 mL methanol was added all at once, and the mixture was heated to reflux 17 hours. The reaction was concentrated in vacuo, dissolved in dichloromethane and washed with water, 0.1 N NaOH and water again. Dried over MgSO₄, condensed, and the residue purified by flash chromatography (0-15-100% ethyl acetate in hexane) to give 1.5 g of 6-iodo-3,3-dimethyl-1-(1,2,4-thiadiazol-5-yl)indoline A.81. 1H NMR (500 MHz, DMSO-d₆) δ ppm 8.39 (1H, s), 8.30 (1H, s), 7.43 (1 H, d, J=7.8 Hz), 7.18 (1H, d, J=7.8 Hz), 3.84 (2H, s), 1.37 (6H, s)

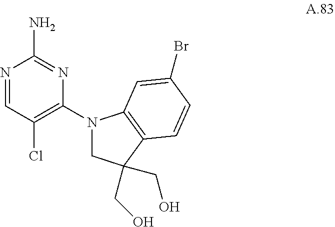

(1-(2-Amino-5-chloro-4-pyrimidinyl)-6-bromo-3-(hydroxymethyl)-2,3-dihydro-1H-indol-3-yl)methanol. A.83 was prepared from A.39 by the procedure used to prepare A.2. MS ESI (pos.) m/e: 385.0 (M+H).

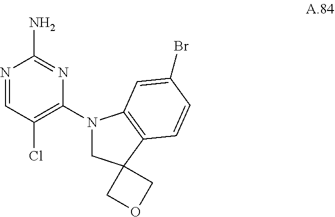

4-(6-bromospiro[indoline-3,3′-oxetane]-1-yl)-5-chloropyrimidin-2-amine A.84. To a sealed tube containing (1-(2-amino-5-chloro-4-pyrimidinyl)-6-bromo-3-(hydroxymethyl)-2,3-dihydro-1H-indol-3-yl)methanol A.83 (200.6 mg, 0.52 mmol) in pyridine (8.00 mL) was added potassium tert-butoxide (64.4 mg, 0.57 mmol) in portions. After 1 hour, para-toluene sulfonyl chloride (119.6 mg, 0.63 mmol) was added. Upon complete addition, additional potassium tert-butoxide (88.0 mg, 0.78 mmol) was added then the mixture was heated to 100° C. After 3 hours, the reaction was carefully quenched with cold water then extracted three times with dichloromethane. After concentrating under reduced pressure, the residue (191 mg) was used as is for the preparation of examples 81 and 82.

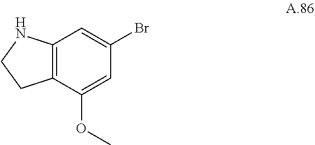

6-bromo-4-methoxyindoline A.86 To a stirred solution of 6-bromo-4-methoxy-1H-indole A.85 (1.80 g, 7.98 mmol) in acetic acid (21 mL) at room temperature was added NaBH₃CN (1.5037 g, 23.93 mmol) and the mixture was stirred at room temperature for 1 hour. To the mixture was added water (100 mL) and the mixture was cooled to 0° C. The mixture was made basic with 10 N aqueous NaOH (35 mL) to pH 14. The mixture was extracted with ether (50 mL×3). The combined organic layers were washed with water (50 mL×2) and brine (50 mL×2), dried over K₂CO₃, filtered, and concentrated under reduced pressure to give 6-bromo-4-methoxyindoline A.86 (1.56 g, 85.7% yield) as a brown oil: ¹H NMR (500 MHz, DMSO-d₆) δ ppm 6.34 (1H, d, J=1.4 Hz), 6.29 (1H, d, J=1.4 Hz), 5.71 (1H, s), 3.72 (3H, s), 3.39-3.46 (2H, m), 2.76 (2H, t, J=8.7 Hz); Mass Spectrum (ESI) m/e=228.1 [M+1 (⁷⁹Br)] and 230.1 [M+1 (⁸¹Br)].

### 4-(6-bromo-4-methoxyindolin-1-yl)-5-fluoropyrimidin-2-amine A.87

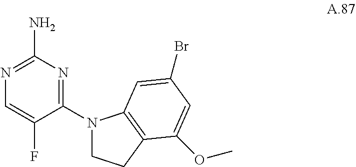

A mixture of 6-bromo-4-methoxyindoline (A.86), (0.8058 g, 5.46 mmol) and 4-chloro-5-fluoropyrimidin-2-amine (A.53), (1 g, 4.38 mmol) in 1,4-dioxane (10 mL) was stirred at 90° C. for 2 hours. The mixture was cooled to room temperature. The resulting precipitate was collected by suction filtration and washed with 1,4-dioxane to give a red solid. The product was purified by silica gel column chromatography using 10% of dichloromethane-methanol-NH₄OH (89:9:1) in dichloromethane as eluent to give 4-(6-bromo-4-methoxyindolin-1-yl)-5-fluoropyrimidin-2-amine (A.87) (0.882 g, 59.3% yield) as a yellow solid: ¹H NMR (500 MHz, DMSO-d₆) δ ppm 8.00 (1H, d, J=6.0 Hz), 7.90 (1H, s), 6.81 (1H, d, J=1.4 Hz), 6.39 (2H, s), 4.19-4.27 (2H, m), 3.81 (3H, s), 2.97 (2H, t, J=8.7 Hz); Mass Spectrum (ESI) m/e=339.0 [M+1 (⁷⁹Br)] and 341.0 [M+1 (⁸¹Br)].

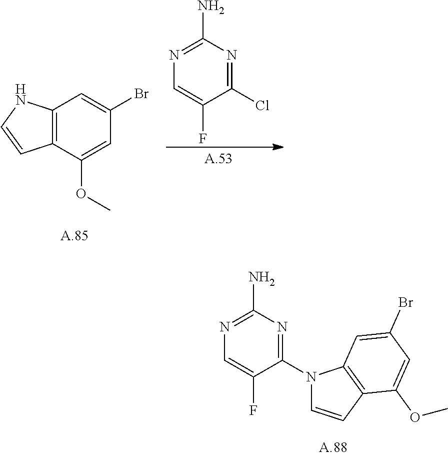

4-(6-bromo-4-methoxy-1H-indol-1-yl)-5-fluoropyrimidin-2-amine A.88 was prepared from components A.53 and A.85 by the procedure described for compound A.134: ¹H NMR (500 MHz, DMSO-d₆) δ ppm 8.45 (1H, d, J=4.1 Hz), 8.07 (1H, s), 7.68 (1H, t, J=3.2 Hz), 6.99 (2 H, s), 6.92 (1H, d, J=1.4 Hz), 6.77 (1H, d, J=3.7 Hz), 3.93 (3H, s); Mass Spectrum (ESI) m/e=337.0 [M+1 (⁷⁹Br)] and 339.0 [M+1 (⁸¹Br)].

### N-(6-bromoindolin-4-yl)acetamide

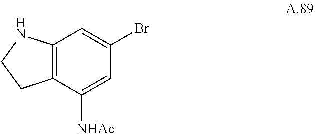

To a stirred solution of N-(6-bromo-1H-indol-4-yl)acetamide A.138 (1.086 g, 4.29 mmol) in acetic acid (10 mL) at room temperature was added NaBH₃CN (0.8092 g, 12.88 mmol) and the mixture was stirred at room temperature for 19 hours. To the mixture was added water (50 mL) and the mixture was cooled to 0° C. The mixture was made basic with 10 N aqueous NaOH (20 mL) to pH 14. The mixture was extracted with ether (50 mL×2). The combined organic layers were washed with water (50 mL×2) and brine (50 mL×1), dried over K₂CO₃, filtered, and concentrated under reduced pressure to give a brown solid. The brown solid was purified by silica gel column chromatography using 90% of ethyl acetate in hexane as eluent to give N-(6-bromoindolin-4-yl)acetamide A.89 (0.6229 g, 56.9% yield) as a tan solid: ¹H NMR (400 MHz, DMSO-d₆) δ ppm 9.25 (1H, s), 7.09 (1H, s), 6.37 (1H, s), 5.75 (1H, s), 3.42 (2H, t, J=9.1 Hz), 2.82 (2H, t, J=8.2 Hz), 2.03 (3H, s)

Mass Spectrum (ESI) m/e=255.0 [M+1 (⁷⁹Br)] and 257.0 [M+1 (⁸¹Br)].

### N-(1-(2-amino-5-chloropyrimidin-4-yl)-6-bromoindolin-4-yl)acetamide

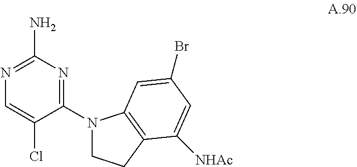

A mixture of N-(6-bromoindolin-4-yl)acetamide A.89 (0.3109 g, 1.22 mmol), 4,5-dichloropyrimidin-2-amine A.14 (0.1999 g, 1.22 mmol), and HCl (1.25 M sol. in EtOH, 0.3 mL, 0.375 mmol) in 1,4-dioxane (5 mL) was stirred at 90° C. for 27 hours. The mixture was concentrated under reduced pressure and purified by silica gel column chromatography using 0% to 100% of dichloromethane-methanol-NH₄OH (89:9:1) in dichloromethane as eluent to give N-(1-(2-amino-5-chloropyrimidin-4-yl)-6-bromoindolin-4-yl)acetamide A.90 (0.3774 g, 80.9% yield) as an orange solid: ¹H NMR (500 MHz, DMSO-d₆) δ ppm 9.50 (1H, s), 8.10 (1 H, s), 7.48 (1H, s), 7.17 (1H, s), 6.68 (2H, s), 4.20 (2H, t, J=8.2 Hz), 2.99 (2H, t, J=8.0 Hz), 2.07 (3H, s); Mass Spectrum (ESI) m/e=382.0 [M+1 (⁷⁹Br)] and 384.0 [M+1 (⁸¹Br)].

### 4-chloro-N-(4-(2-methoxyethoxy)phenyl)-1,3,5-triazin-2-amine

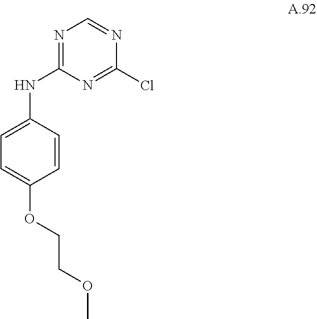

To a solution of 2.4-dichloro-1,3,5-triazine A.91 (2 g, 13.34 mmol) in DMF (10 mL) at 0° C. were added DIEA (2.4 mL, 13.77 mmol) and 4-(2-methoxyethoxy)benzenamine (2.027 g, 12.12 mmol) and the mixture was stirred at 0° C. for 30 minutes and then room temperature for 1 hour. The mixture was diluted with ethyl acetate (100 mL) and washed with brine (50 mL×1), dried over Na₂SO₄, filtered, and concentrated under reduced pressure to give a brown solid. The brown solid was purified by silica gel column chromatography using 30% of ethyl acetate in hexane as eluent to give 4-chloro-N-(4-(2-methoxyethoxy)phenyl)-1,3,5-triazin-2-amine A.92 (2.887 g, 84.8% yield) as a white solid: ¹H NMR (400 MHz, DMSO-d₆) δ ppm 10.58 (1 H, s), 8.56 (1H, d, J=5.9 Hz), 7.51 (2H, t, J=9.5 Hz), 6.96 (2H, t, J=7.9 Hz), 4.04-4.11 (2H, m), 3.65 (2H, dd, J=5.3, 3.8 Hz), 3.31 (3H, s); Mass Spectrum (ESI) m/e=281.0 [M+1].

### 4-(6-bromo-3,3-dimethylindolin-1-yl)-N-(4-(2-methoxyethoxy)phenyl)-1,3,5-triazin-2-amine

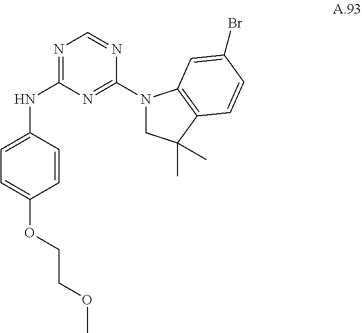

A mixture of 4-chloro-N-(4-(2-methoxyethoxy)phenyl)-1,3,5-triazin-2-amine A.92 (0.2483 g, 0.885 mmol), 6-bromo-3,3-dimethylindoline A.54 (0.2 g, 0.885 mmol), and 1N aq. HCl (10 mL) was heated at reflux with stirring for 30 minutes. The mixture was cooled to 0° C. and neutralized with 2 N aq. NaOH to pH 8.0 with stirring. The aqueous layer was extracted with ethyl acetate (50 mL×3). The combined organic layers were washed with brine (50 mL×1), dried over Na₂SO₄, filtered, and concentrated under reduced pressure to give a brown solid. The product was purified by silica gel column chromatography using 30% to 50% gradient of ethyl acetate in hexane as eluent to give 4-(6-bromo-3,3-dimethylindolin-1-yl)-N-(4-(2-methoxyethoxy)phenyl)-1,3,5-triazin-2-amine A.93 (0.1825 g, 43.9% yield) as a solid: ¹H NMR (400 MHz, DMSO-d₆) δ ppm 9.74 (1H, s), 8.09-8.58 (2H, m), 7.49 (2H, d, J=46.1 Hz), 7.08-7.26 (2H, m), 6.95 (2H, d, J=4.4 Hz), 4.05-4.11 (2H, m), 3.95 (2H, s), 3.62-3.68 (2H, m), 3.30 (3H, s), 1.31 (6H, s); Mass Spectrum (ESI) m/e=470.0 [M+1 (⁷⁹Br)] and 472.1 [M+1 (⁸¹Br)].

### 4-(6-bromoindolin-1-yl)pyrimidin-2-amine

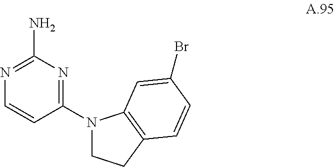

A mixture of 6-bromoindoline A.1 (3.96 g, 20 mmol) and 4-chloropyrimidin-2-amine A.12 (3.3 g, 20 mmol) in 1,4-dioxane (80 mL) was stirred at 90° C. for 21 hours. The mixture was cooled to room temperature. The resulting precipitate was collected by filtration and washed with ether (60 mL) to give 4-(6-bromoindolin-1-yl)pyrimidin-2-amine A.95 (5.35 g, 92% yield) as a yellow solid: ¹H NMR (500 MHz, DMSO-d₆) δ ppm 8.72 (1H, s), 8.06 (1H, d, J=7.1 Hz), 7.36 (1H, s), 7.26 (2H, s), 7.16 (1H, s), 6.42 (1H, d, J=7.1 Hz), 4.18 (2H, t, J=8.4 Hz), 3.19 (2H, t, J=7.7 Hz); Mass Spectrum (ESI) m/e=291.1 [M+1 (⁷⁹Br)] and 293.0 [M+1 (⁸¹Br)].

### Methyl (4-bromo-2-nitrophenyl)acetate A.97

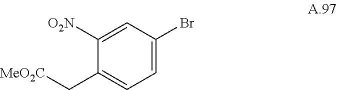

Ref. Quallich, G. J.; Morrissey, P. M. Synthesis 1993, 51-53.

To a stirred solution of dimethyl 2-(4-bromo-2-nitrophenyl)malonate A.17 (20 g, 60.2 mmol) in DMSO (200 mL) was added lithium chloride (5.11 g, 2 eq.) followed by water (1.1 mL, 1 eq.) at room temperature. The resulting mixture was stirred at 100° C. for 2 days and at ambient temperature for 4 more days. The mixture was poured into water and extracted with ethyl acetate (2×). The combined organics were washed with brine (2×) and dried over Na₂SO₄. The residue after concentration in vacuo was subjected to Combi-Flash column chromatography (ethyl acetate/hexanes) to give methyl (4-bromo-2-nitrophenyl)acetate A.97 (12 g, 73%) as a light yellow solid. ¹H NMR (400 MHz, DMSO-d₆) δ 8.30 (d, J=2.1 Hz, 1H), 7.98 (dd, J=8.2, 2.1 Hz, 1H), 7.56 (d, J=8.2 Hz, 1H), 4.08 (s, 2H), 3.63 (s, 3H). LCMS-ESI (POS), M/Z, M+1: Found 274.0 and 276.0.

### methyl 4-(4-bromo-2-nitrophenyl)-tetrahydro-2H-pyran-4-carboxylate

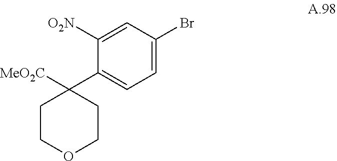

Sodium hydride (0.32 g, 8.03 mmol, 60% dispersion in oil) was added in portions at room temperature to a stirred solution of methyl 2-(4-bromo-2-nitrophenyl)acetate A.97 (1 g, 3.65 mmol) in DMSO (15 mL). After the mixture was stirred at room temperature for 30 min, sodium iodide (0.055 g, 0.365 mmol) and bis(2-bromoethyl)ether (1.27 g, 5.47 mmol) were added. The resultant mixture was stirred at 40° C. After 19 hours, the mixture was poured into brine with ice (˜50 mL), extracted with ethyl acetate (50 mL, 3 times). The combined extracts were washed with brine (80 mL, 3 times), dried (Na₂SO₄), filtered, and concentrated under reduced pressure to give 1.69 g of a dark liquid. The product was purified by silica gel column chromatography using 0 to 50% gradient of ethyl acetate in n-hexane to give methyl 4-(4-bromo-2-nitrophenyl)-tetrahydro-2H-pyran-4-carboxylate A.98 (0.45 g, 36%) as an orange syrup: ¹H NMR (500 MHz, CHLOROFORM-d) δ ppm 7.89 (1H, d, J=2.2 Hz), 7.75 (1H, dd, J=8.6, 2.2 Hz), 7.50 (1H, d, J=8.8 Hz), 3.87-3.94 (2H, m), 3.66-3.77 (5H, m), 2.33 (2H, dd, J=14.1, 2.8 Hz), 1.99-2.05 (2H, m); ESI-MS m/z 344.0 [(M+1) (⁷⁹Br)] and 346.0 [(M+1) (⁸¹Br)].

### 6-bromo-2′,3′,5′,6′-tetrahydrospiro[indole-3,4′-pyran]-2(1H)-one

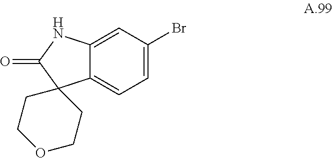

Methyl 4-(4-bromo-2-nitrophenyl)-tetrahydro-2H-pyran-4-carboxylate A.98 (6.67 g, 19.4 mmol) was dissolved in acetic acid (97 mL), Fe powder (5.42 g, 96.97 mmol) was added and the resultant mixture was stirred at 100° C. for 2.5 hours. The hot mixture was filtered through a Celite pad and the pad was washed with acetic acid (250 mL). The filtrate was concentrated under reduced pressure to give a dark syrup. The product was purified by silica gel column chromatography using 0 to 100% gradient of ethyl acetate in n-hexane) to give 4.75 g of a reddish-pink solid. The solid was suspended in 50% of ethyl acetate in hexane (20 mL), filtered, and washed with 50% of ethyl acetate in hexane (40 mL) on the filter paper to give 6-bromo-2′,3′,5′,6′-tetrahydrospiro[indole-3,4′-pyran]-2(1H)-one A.99 (3.93 g, 72%) as an orange-pink solid: ¹H NMR (500 MHz, DMSO-d₆) δ ppm 10.53 (1H, s), 7.47 (1H, d, J=7.8 Hz), 7.14 (1H, dd, J=8.1, 1.0 Hz), 6.98 (1H, d, J=1.0 Hz), 3.91-4.12 (2H, m), 3.67-3.86 (2 H, m), 1.54-1.84 (4H, m); ESI-MS m/z 282.0 [(M+1) (⁷⁹Br)] and 284.0 [(M+1) (⁸¹Br)].

### 6-bromo-1,2,2′,3′,5′,6′-hexahydrospiro[indole-3,4′-pyran] A.100

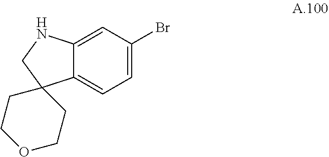

A heterogeneous mixture of 6-bromo-2′,3′,5′,6′-tetrahydrospiro[indole-3,4′-pyran]-2(1H)-one A.99 (3.5 g, 12.4 mmol) in toluene (25 mL) was stirred at 80° C. To the heated mixture was added a solution of Red-Al (65% in toluene, 11.6 mL, 37.2 mmol) and the mixture was stirred at 80° C. After 50 min, the mixture was cooled to 0° C. and quenched with 2 N of aqueous NaOH (31 mL, 62 mmol) with stirring. The mixture was extracted with ethyl acetate (100 mL, 2 times). The combined extracts were washed with brine (100 mL, 3 times), dried (Na₂SO₄), filtered, and concentrated under reduced pressure to give a green solid. The product was purified by silica gel column chromatography using 0 to 100% gradient of dichloromethane-methanol-NH₄OH (89:9:1) in dichloromethane) to give 6-bromo-1,2,2′,3′,5′,6′-hexahydrospiro[indole-3,4′-pyran] A.100 (2.13 g, 64%) as a yellow solid: ¹H NMR (500 MHz, DMSO-d₆) δ ppm 6.91-6.99 (1H, m), 6.66 (1H, dd, J=7.8, 1.7 Hz), 6.59 (1 H, d, J=1.7 Hz), 5.85 (1H, s), 3.70-3.87 (2H, m), 3.36-3.51 (4H, m), 1.65-1.84 (2H, m), 1.39-1.57 (2H, m); ESI-MS m/z 268.0 [(M+1) (⁷⁹Br)] and 270.0 [(M+1) (⁸¹Br)].

### 4-(6-bromo-2′,3′,5′,6′-tetrahydrospiro[indole-3,4′-pyran]-1(2H)-yl)-5-chloro-2-pyrimidinamine

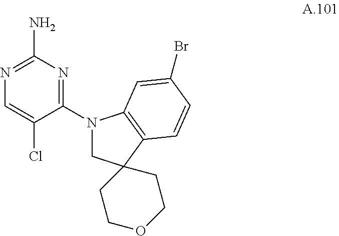

A mixture of 6-bromo-1,2,2′,3′,5′,6′-hexahydrospiro[indole-3,4′-pyran]A.100 (1 g, 3.73 mmol) and 4,5-dichloropyrimidin-2-amine A.14 (1.223 g, 7.46 mmol) in 1 N aqueous HCl (60 mL) was stirred at 60° C. for 18.5 hours. The mixture was cooled to room temperature and poured into 2 N aqueous NaOH (180 mL) with ice. The mixture was extracted with ethyl acetate (100 mL×2). The combined organic layers were washed with brine (100 mL×2), dried over Na₂SO₄, filtered, and concentrated under reduced pressure to give a pink solid. The pink solid was purified by silica gel column chromatography using 0% to 100% gradient of ethyl acetate in hexane as eluent to give 4-(6-bromo-2′,3′,5′,6′-tetrahydrospiro[indole-3,4′-pyran]-1(2H)-yl)-5-chloro-2-pyrimidinamine A.101 (1.17 g, 79.4% yield) as a light yellow solid: ¹H NMR (500 MHz, DMSO-d₆) δ ppm 8.08-8.15 (1H, m), 7.36 (1H, s), 7.17-7.27 (1H, m), 7.03-7.13 (1 H, m), 6.73 (2H, s), 4.16 (2H, s), 3.84 (2H, dd, J=11.6, 3.1 Hz), 3.46 (2H, t, J=11.5 Hz), 1.76-1.93 (2H, m), 1.56 (2H, d, J=13.2 Hz); Mass Spectrum (ESI) m/e=395.0 [M+1 (⁷⁹Br)] and 397.0 [M+1 (⁸¹Br)].

### 6-bromo-1-(5-chloropyrimidin-4-yl)-1,2,2′,3′,5′,6′-hexahydrospiro[indole-3,4′-pyran]

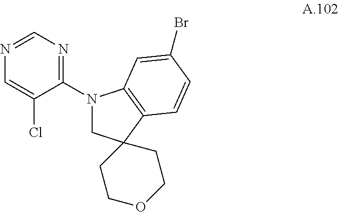

To a heterogeneous mixture of 4-(6-bromo-2′,3′,5′,6′-tetrahydrospiro[indole-3,4′-pyran]-1(2H)-yl)-5-chloro-2-pyrimidinamine A.101 (0.2 g, 0.505 mmol) in THF (1 mL) was added dropwise isoamylnitrite (0.34 mL, 2.53 mmol) with stirring and the mixture was heated at reflux for 1 hour. The mixture was poured into ice water (30 mL) and extracted with ethyl acetate (50 mL×2). The combined organic layers were washed with brine (50 mL×1), dried over MgSO₄, filtered, and concentrated under reduced pressure to give a brown solid. The product was purified by silica gel column chromatography using 0% to 100% gradient of ethyl acetate in hexane as eluent to give 6-bromo-1-(5-chloropyrimidin-4-yl)-1,2,2′,3′,5′,6′-hexahydrospiro[indole-3,4′-pyran] A.102 (0.062 g, 32.2% yield) as a brown solid: ¹H NMR (500 MHz, DMSO-d₆) δ ppm 8.74 (1H, s), 8.68 (1H, s), 7.63 (1H, d, J=1.2 Hz), 7.30 (1H, d, J=8.1 Hz), 7.18 (1H, dd, J=7.9, 1.1 Hz), 4.23-4.37 (2H, m), 3.84 (2H, dd, J=11.9, 3.3 Hz), 3.48 (2H, t, J=11.4 Hz), 1.83-1.95 (2H, m), 1.58 (2H, d, J=13.0 Hz); Mass Spectrum (ESI) m/e=380.0 [M+1 (⁷⁹Br)] and 382.0 [M+1 (⁸¹Br)].

### 1-(5-chloropyrimidin-4-yl)-6-iodo-1,2,2′,3′,5′,6′-hexahydrospiro[indole-3,4′-pyran]

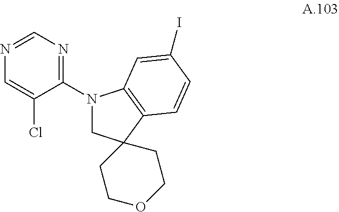

To a Schlenk tube were added 6-bromo-1-(5-chloropyrimidin-4-yl)-1,2,2′,3′,5′,6′-hexahydrospiro[indole-3,4′-pyran] A.102 (0.062 g, 0.16 mmol), CuI (0.0155 g, 0.081 mmol), NaI (0.049 g, 0.326 mmol), N1,N2-dimethylethane-1,2-diamine (0.018 mL, 0.167 mmol), and 1,4-dioxane (0.33 mL) under argon and the mixture was stirred at 110° C. for 16 hours. The mixture was cooled to room temperature, diluted with 30% of aqueous ammonia (5 mL), poured into water (20 mL), and extracted with dichloromethane (15 mL×3). The combined organic layers were dried over MgSO₄, filtered, and concentrated under reduced pressure to give a yellow solid. The yellow solid was purified by silica gel column chromatography using 0% to 100% gradient of ethyl acetate in hexane as eluent to give 1-(5-chloropyrimidin-4-yl)-6-iodo-1,2,2′,3′,5′,6′-hexahydrospiro[indole-3,4′-pyran] A.103 (0.0384 g, 55% yield) as a solid:

¹H NMR (500 MHz, DMSO-d₆) δ ppm 8.74 (1H, s), 8.67 (1H, s), 7.77 (1H, s), 7.35 (1H, d, J=8.6 Hz), 7.15 (1H, d, J=7.8 Hz), 4.29 (2H, s), 3.76-3.91 (2H, m), 3.41-3.52 (2H, m), 1.87 (2H, d, J=4.4 Hz), 1.57 (2H, d, J=13.4 Hz); Mass Spectrum (ESI) m/e=427.9 [M+1].

### 4-(6-bromo-2′,3′,5′,6′-tetrahydrospiro[indole-3,4′-pyran]-1(2H)-yl)pyrimidin-2-amine

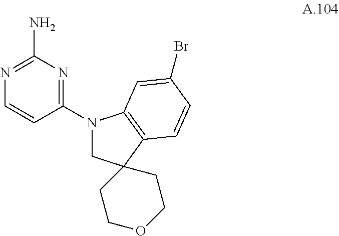

A mixture of 6-bromo-1,2,2′,3′,5′,6′-hexahydrospiro[indole-3,4′-pyran] A.100 (0.5 g, 1.86 mmol) and 4-chloropyrimidin-2-amine A.12 (0.4831 g, 3.73 mmol) in 1 N aqueous HCl (30 mL) was stirred at 60° C. for 17.5 hours. The mixture was cooled to room temperature and poured into 2 N aqueous NaOH (90 mL) with ice. The mixture was extracted with ethyl acetate (50 mL×2). The combined organic layers were washed with brine (100 mL×2), dried over Na₂SO₄, filtered, and concentrated under reduced pressure to give a pink solid. The pink solid was purified by silica gel column chromatography using 0% to 100% gradient of dichloromethane-methanol-NH₄OH (89:9:1) in dichloromethane as eluent to give 4-(6-bromo-2′,3′,5′,6′-tetrahydrospiro[indole-3,4′-pyran]-1(2H)-yl)pyrimidin-2-amine A.104 (0.595 g, 88.3% yield) as a white solid: ¹H NMR (500 MHz, DMSO-d₆) δ ppm 8.65 (1H, d, J=1.5 Hz), 8.00 (1H, d, J=5.9 Hz), 7.21 (1H, d, J=8.1 Hz), 7.09 (1H, dd, J=7.6, 1.5 Hz), 6.44 (2H, s), 6.18 (1H, d, J=5.9 Hz), 3.96 (2H, s), 3.85 (2H, dd, J=11.7, 3.7 Hz), 3.55 (2H, t, J=11.7 Hz), 1.73-1.98 (2H, m), 1.54 (2H, d, J=13.0 Hz); Mass Spectrum (ESI) m/e=361.0 [M+1 (⁷⁹Br)] and 363.1 [M+1 (⁸¹Br)].

### 6-bromo-1-pyrimidin-4-yl-1,2,2′,3′,5′,6′-hexahydrospiro[indole-3,4′-pyran]

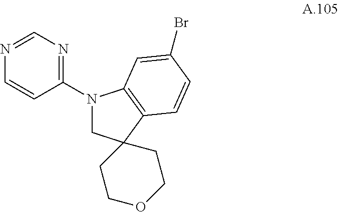

To a heterogeneous mixture of 4-(6-bromo-2′,3′,5′,6′-tetrahydrospiro[indole-3,4′-pyran]-1(2H)-yl)pyrimidin-2-amine A.104 (0.3 g, 0.0.83 mmol) in THF (1.7 mL) was added dropwise isoamylnitrite (0.56 mL, 4.15 mmol) with stirring and the mixture was heated at reflux for 45 hours. The mixture was poured into ice water (50 mL) and extracted with ethyl acetate (50 mL×3). The combined organic layers were washed with brine (100 mL×1), dried over MgSO₄, filtered, and concentrated under reduced pressure to give a brown solid. The product was purified by silica gel column chromatography using 0% to 100% gradient of dichloromethane-methanol-NH₄OH (89:9:1) in dichloromethane as eluent to give 6-bromo-1-pyrimidin-4-yl-1,2,2′,3′,5′,6′-hexahydrospiro[indole-3,4′-pyran] A.105 (0.0631 g, 21.9% yield): ¹H NMR (500 MHz, DMSO-d₆) δ ppm 8.82 (1H, s), 8.67 (1H, d, J=1.7 Hz), 8.49 (1H, d, J=6.1 Hz), 7.29 (1 H, d, J=8.1 Hz), 7.18 (1H, dd, J=7.9, 1.8 Hz), 7.03 (1H, d, J=6.1 Hz), 4.07 (2H, s), 3.87 (2H, dd, J=11.7, 3.7 Hz), 3.57 (2H, t, J=11.5 Hz), 1.79-2.01 (2H, m), 1.58 (2H, d, J=13.0 Hz); Mass Spectrum (ESI) m/e=346.0 [M+1 (⁷⁹Br)] and 348.1 [M+1 (⁸¹Br)].

### 6-iodo-1-pyrimidin-4-yl-1,2,2′,3′,5′,6′-hexahydrospiro[indole-3,4′-pyran]

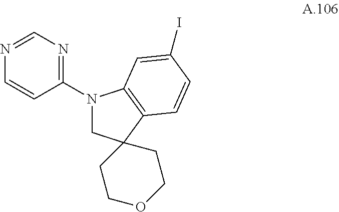

To a Schlenk tube were added 6-bromo-1-pyrimidin-4-yl-1,2,2′,3′,5′,6′-hexahydrospiro[indole-3,4′-pyran] A.105 (0.0595 g, 0.172 mmol), CuI (0.0164 g, 0.086 mmol), NaI (0.0515 g, 0.344 mmol), N1,N2-dimethylethane-1,2-diamine (0.0185 mL, 0.172 mmol), and 1,4-dioxane (0.34 mL) under argon and the mixture was stirred at 110° C. for 21.5 hours. The mixture was cooled to room temperature, diluted with 30% of aqueous ammonia (5 mL), poured into water (20 mL), and extracted with dichloromethane (15 mL×3). The combined organic layers were dried over MgSO₄, filtered, and concentrated under reduced pressure to give a yellow solid. The yellow solid was purified by silica gel column chromatography using 0% to 100% gradient of dichloromethane-methanol-NH₄OH (89:9:1) in dichloromethane as eluent to give 6-iodo-1-pyrimidin-4-yl-1,2,2′,3′,5′,6′-hexahydrospiro[indole-3,4′-pyran] A.106 (0.0464 g, 68.7% yield) as a solid: ¹H NMR (500 MHz, DMSO-d₆) δ ppm 8.85 (1H, d, J=1.5 Hz), 8.81 (1H, s), 8.48 (1 H, d, J=5.9 Hz), 7.35 (1H, dd, J=7.8, 1.5 Hz), 7.15 (1H, d, J=7.8 Hz), 7.02 (1H, d, J=5.6 Hz), 4.04 (2H, s), 3.87 (2H, dd, J=11.6, 4.0 Hz), 3.57 (2H, t, J=11.4 Hz), 1.84-1.95 (2H, m), 1.57 (2H, d, J=13.2 Hz); Mass Spectrum (ESI) m/e=394.0 [M+1].

### methyl 2-(4-bromo-2-nitrophenyl)acrylate

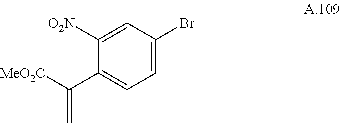

Methyl 2-(4-bromo-2-nitrophenyl)acrylate A.109 was prepared by the method of Selvakumar, N.; Azhagan, A. M.; Srinivas, D.; Krishna, G. G. Tetrahedron Lett. 2002, 43, 9175-9178.

To a stirred mixture of dimethyl 2-(4-bromo-2-nitrophenyl)malonate A.17 (8 g, 24.23 mmol) and formaldehyde (37 wt. % in water) (45 mL) was added a solution of K₂CO₃ (6.3 g, 1.9 eq.) in water (20 mL). The resulting mixture was stirred in an oil bath preheated at 60° C. for 6 h and then cooled to rt. The reaction mixture was poured into water, extracted with ethyl acetate (2×). The combined organic extracts were washed with brine (2×) and dried over Na₂SO₄. The residue after evaporation in vacuo was purified on a column of silica gel (ethyl acetate/hexanes) to afford methyl 2-(4-bromo-2-nitrophenyl)acrylate (8.5 g, 98% yield) as a colorless oil. ¹H NMR (400 MHz, DMSO-d₆) δ ppm 8.31 (1H, d, J=2.2 Hz), 8.03 (1H, dd, J=8.2, 2.0 Hz), 7.51 (1H, d, J=8.1 Hz), 6.49 (1H, s), 6.14 (1H, s), 3.65 (3H, s). LCMS-ESI (POS), M/Z, M+Na⁺: Found 308.2 and 310.2.

### methyl 2-(4-bromo-2-nitrophenyl)oxirane-2-carboxylate

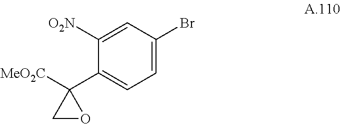

A solution of methyl 2-(4-bromo-2-nitrophenyl)acrylate A.109 (1.8603 g, 6.5 mmol) in chloroform (13 mL) was treated with 77% of m-CPBA (3.6434 g, 16.26 mmol) and heated at reflux for 48 hours. The mixture was cooled to room temperature, diluted with chloroform (150 mL), washed with 10% aqueous NaHSO₃ (100 mL), 10% aqueous NaHCO₃ (100 mL), and water (100 mL). The organic layer was dried over MgSO₄, filtered, and concentrated under reduced pressure to give a yellow oil. The yellow oil was purified by silica gel column chromatography using 0% to 70% gradient of ethyl acetate in hexane as eluent to give methyl 2-(4-bromo-2-nitrophenyl)oxirane-2-carboxylate A.110 (1.4664 g, 74.6% yield): ¹H NMR (500 MHz, DMSO-d₆) δ ppm 8.40 (1H, d, J=2.0 Hz), 8.07 (1H, dd, J=8.3, 2.0 Hz), 7.67 (1H, d, J=8.3 Hz), 3.62-3.68 (3H, m), 3.59 (1H, d, J=5.6 Hz), 3.23 (1H, d, J=5.9 Hz); Mass Spectrum (ESI) m/e=301.9 [M+1 (⁷⁹Br)] and 304.0 [M+1 (⁸¹Br)].

### methyl 2-(4-bromo-2-nitrophenyl)-4-methylmorpholine-2-carboxylate

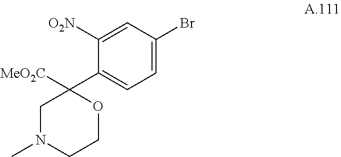

A mixture of methyl 2-(4-bromo-2-nitrophenyl)oxirane-2-carboxylate A.110 (1.4633 g, 4.84 mmol), THF (4.8 mL), and 2-(methylamino)ethanol (0.58 mL, 7.2 mmol) was stirred at 50° C. for 27 hours. The mixture was concentrated under reduced pressure to give methyl 2-(4-bromo-2-nitrophenyl)-2-hydroxy-3-((2-hydroxyethyl)(methyl)amino)propanoate as a dark red syrup: Mass Spectrum (ESI) m/e=377.0 [M+1 (⁷⁹Br)] and 379.0 [M+1 (⁸¹Br)]. The product was carried on to the next step. Methyl 2-(4-bromo-2-nitrophenyl)-2-hydroxy-3-((2-hydroxyethyl)(methyl)amino)propanoate (˜4.84 mmol) was dissolved in 1,4-dioxane (7 mL), and powdery KOH (1.0863 g, 19.36 mmol) and tris(3,6-dioxaheptyl)amine (0.019 g, 0.058 mmol) were added. To the mixture was added dropwise with stirring a solution of p-TsCl (1.2918 g, 6.78 mmol) in 1,4-dioxane (3 mL) over 3 minutes and the mixture was stirred at room temperature for 4 hours. The mixture was filtered. The filtrate was concentrated under reduced pressure and purified by silica gel column chromatography using 0% to 100% gradient of ethyl acetate in hexane as eluent to give methyl 2-(4-bromo-2-nitrophenyl)-4-methylmorpholine-2-carboxylate A.111 (0.5133 g, 29.5% overall yield over two steps) as a yellow solid: ¹H NMR (400 MHz, DMSO-d₆) δ ppm 8.04 (1H, d, J=2.0 Hz), 7.87 (1H, dd, J=8.6, 2.0 Hz), 7.64 (1H, d, J=8.6 Hz), 3.68-3.74 (1H, m), 3.67 (3H, s), 3.16-3.27 (2H, m), 2.54-2.65 (1H, m), 2.30-2.38 (1H, m), 2.16-2.26 (4H, m); Mass Spectrum (ESI) m/e=359.0 [M+1 (⁷⁹Br)] and 361.0 [M+1 (⁸¹Br)].

### 6-bromo-4′-methylspiro[indole-3,2′-[1,4]oxazinan]-2(1)-one

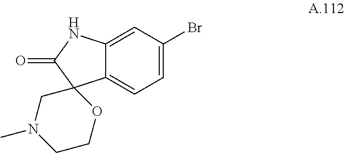

A mixture of methyl 2-(4-bromo-2-nitrophenyl)-4-methylmorpholine-2-carboxylate A.111 (0.5133 g, 1.43 mmol), acetic acid, and Fe (0.3991 g, 7.15 mmol) was stirred at 100° C. for 2 hours. The hot mixture was filtered through a Celite pad and the pad was washed with acetic acid (100 mL). The filtrate was concentrated under reduced pressure and purified by silica gel column chromatography using 0% to 90% gradient of ethyl acetate in hexane as eluent to give 6-bromo-4′-methylspiro[indole-3,2′-[1,4]oxazinan]-2(1H)-one A.112 (0.2645 g, 62.3% yield) as a white syrup: ¹H NMR (500 MHz, DMSO-d₆) δ ppm 10.58 (1H, s), 7.64 (1 H, d, J=7.8 Hz), 7.17 (1H, dd, J=8.1, 1.7 Hz), 6.99 (1H, d, J=1.5 Hz), 3.93 (2H, dd, J=6.5, 3.1 Hz), 2.67 (1H, d, J=11.7 Hz), 2.51-2.55 (1H, m), 2.39 (1H, d, J=11.5 Hz), 2.27-2.34 (1 H, m), 2.19 (3H, s); Mass Spectrum (ESI) m/e=297.0 [M+1 (⁷⁹Br)] and 298.9 [M+1 (⁸¹Br)].

### 6-bromo-4′-methyl-1,2-dihydrospiro[indole-3,2′-[1,4]oxazinane]

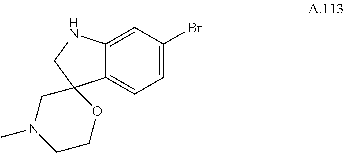

A heterogeneous mixture of 6-bromo-4′-methylspiro[indole-3,2′-[1,4]oxazinan]-2(1H)-one A.112 (0.9598 g, 3.23 mmol) in toluene (6.5 mL) was stirred at 80° C. A solution of Red-Al in toluene (3.2 M, 3 mL, 9.69 mmol) was added dropwise at 80° C. and the mixture was stirred at 80° C. for 40 minutes. The mixture was cooled to 0° C., quenched with 2 N aqueous NaOH (8 mL, 16 mmol), and extracted with ethyl acetate (50 mL×2). The combined organic layers were washed with brine (50 mL×1), dried over Na₂SO₄, filtered, and concentrated under reduced pressure to give a violet syrup. The product was purified by silica gel column chromatography using 0% to 100% gradient of dichloromethane-methanol-NH₄OH (89:9:1) in dichloromethane as eluent to give 6-bromo-4′-methyl-1,2-dihydrospiro[indole-3,2′-[1,4]oxazinane] A.113 (0.4841 g, 52.9% yield) as a light yellow solid: ¹H NMR (400 MHz, DMSO-d₆) δ ppm 7.24 (1H, d, J=8.2 Hz), 6.63-6.71 (2H, m), 5.97 (1H, s), 3.54-3.70 (2H, m), 3.39-3.49 (2H, m), 2.25-2.47 (4H, m), 2.19 (3H, s); Mass Spectrum (ESI) m/e=283.0 [M+1 (⁷⁹Br)] and 285.0 [M+1 (⁸¹Br)].

### 4-(6-bromo-4′-methylspiro[indole-3,2′-[1,4]oxazinan]-1(2H)-yl)-5-chloropyrimidin-2-amine

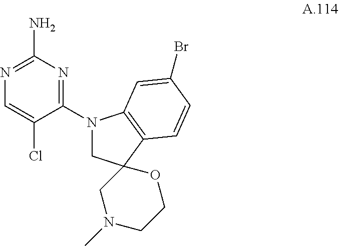

A mixture of 6-bromo-4′-methyl-1,2-dihydrospiro[indole-3,2′-[1,4]oxazinane] A.113 (0.4149 g, 2.53 mmol) and 4,5-dichloropyrimidin-2-amine A.14 (0.3582 g, 1.265 mmol) in in 1 N aqueous HCl (20 mL) was stirred at 60° C. for 19 hours. The mixture was cooled to room temperature and poured into 2 N aqueous NaOH (60 mL) with ice. The mixture was extracted with ethyl acetate (50 mL×2). The combined organic layers were washed with brine (50 mL×2), dried over Na₂SO₄, filtered, and concentrated under reduced pressure to give a yellow solid. The yellow solid was purified by silica gel column chromatography using 0% to 100% gradient of dichloromethane-methanol-NH₄OH (89:9:1) in dichloromethane as eluent to give 4-(6-bromo-4′-methylspiro[indole-3,2′-[1,4]oxazinan]-1(2H)-yl)-5-chloropyrimidin-2-amine A.114 (0.1595 g, 30.7% yield) as an orange pink syrup: ¹H NMR (500 MHz, DMSO-d₆) δ ppm 8.12 (1H, s), 7.61 (1H, d, J=1.7 Hz), 7.49 (1H, d, J=7.8 Hz), 7.14 (1H, dd, J=8.1, 1.7 Hz), 6.73 (2H, s), 4.10-4.27 (2H, m), 3.57-3.74 (2H, m), 2.42-2.61 (3H, m), 2.32 (1H, s), 2.22 (3H, s); Mass Spectrum (ESI) m/e=410.0 [M+1 (⁷⁹Br)] and 412.0 [M+1 (⁸¹Br)].

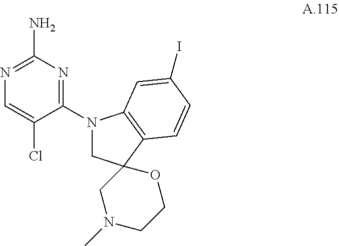

### 5-chloro-4-(6-iodo-4′-methylspiro[indole-3,2′-[1,4]oxazinan]-1(2H)-yl)pyrimidin-2-amine A.115

4-(6-bromo-4′-methylspiro[indole-3,2′-[1,4]oxazinan]-1(2H)-yl)-5-chloropyrimidin-2-amine A.114 (0.1 g, 0.243 mmol), CuI (0.023 g, 0.122 mmol) and NaI (0.073 g, 0.487 mmol) were added to a Schlenk tube, evacuated and backfilled with argon. Under positive pressure of argon, the screw cap was replaced with a septum. N,N′-dimethylethylenediamine (0.026 mL, 0.243 mmol) in 1,4-dioxane (3 mL) was added via syringe, and the screwcap was returned under positive pressure of argon. The reaction was sealed and heated to 110° C. with stirring for 20 hours. The mixture was cooled to room temperature, diluted with 30% aqueous NH₃ (5 mL), and poured into water (20 mL). The solution was extracted with dichloromethane (15 mL×3) and the combined extracts were dried over MgSO₄, filtered, and concentrated under reduced pressure. The product was purified by silica gel column chromatography using 0% to 100% gradient of dichloromethane-methanol-NH₄OH (89:9:1) in dichloromethane as eluent to give 5-chloro-4-(6-iodo-4′-methylspiro[indole-3,2′-[1,4]oxazinan]-1(2H)-yl)pyrimidin-2-amine A.115 (0.1023 g on crude) as a brown syrup: Mass Spectrum (ESI) m/e=458.0 [M+1].

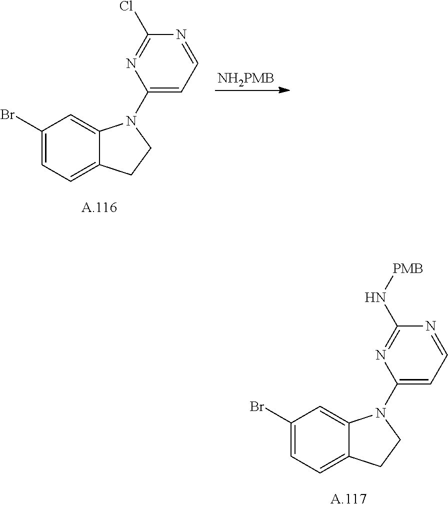

N-(4-methoxybenzyl)-4-(6-bromoindolin-1-yl)pyrimidin-2-amine (A.117): A mixture of 6-bromo-1-(2-chloropyrimidin-4-yl)indoline A.116 (160 mg, 0.52 mmol)(prepared in analogy to compound A.2), PMBNH₂ (0.10 mL, 0.78 mmol) and K₂CO₃ (107.8 mg, 0.78 mmol) in THF (15 mL) was refluxed for 4 days. After cooled to room temperature, the mixture was diluted with ether, washed with water and brine, dried and concentrated. The residue was triturated in dichloromethane and the follow up filtration gave N-(4-methoxybenzyl)-4-(6-bromoindolin-1-yl)pyrimidin-2-amine A.117 (148.2 mg, 69%): ¹H NMR (DMSO-d₆) δ 8.62 (s, 1H), 8.03 (d, J=6.0 Hz, 1H), 7.52 (dd, J=6.0, 6.0 Hz, 1H), 7.33-7.26 (m, 2H), 7.15 (d, J=8.0 Hz, 1H), 7.05 (d, J=8.0 Hz, 1H), 6.86 (d, J=8.0 Hz, 2H), 6.02 (br, 1H), 4.48 (d, J=6.5 Hz, 2H), 3.99 (t, J=8.5 Hz, 2H), 3.71 (s, 3H), 3.13 (t, J=8.5 Hz, 2H); ms 411.1 (M+H⁺).

### 4-(2-(2-(methylthio)pyrimidin-4-yl)acetyl)benzonitrile

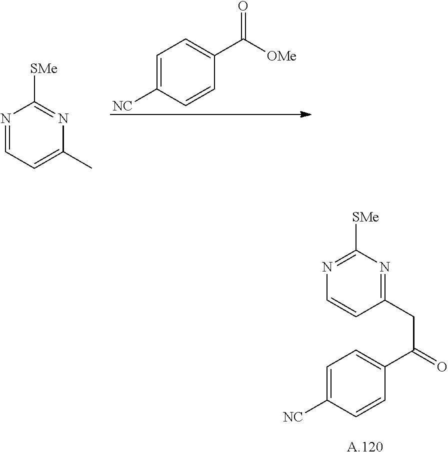

To a solution of sodium bis(trimethylsilyl) amide (2 M sol. in THF, 79 mL) was added a solution of 4-methyl-2-(methylthio)pyrimidine (10 g, 71.32 mmol) in THF (20 mL) at 0° C., and the resulting solution was stirred at 0° C. for 40 minutes. A solution of methyl 4-cyanobenzoate (11.49 g, 71.32 mmol) in THF (30 mL) was then added dropwise over 10 minutes to the mixture, and the mixture was stirred while attaining ambient temperature over 2 hours. The reaction was quenched by addition of sat'd aq. NH₄Cl (100 mL) and extracted into ethyl acetate (250 mL×2), and the combined organic layers were washed with brine (200 mL×1), dried over Na₂SO₄, filtered and concentrated under reduced pressure to give a red solid. The red solid was suspended in hexane (150 mL), sonicated, filtered, and washed with hexane (200 mL) to give 4-(2-(2-(methylthio)pyrimidin-4-yl)acetyl)benzonitrile (A.120) (17.43 g, 90.8% yield) as an orange solid. The product was carried on without purification for the next step: Mass Spectrum (ESI) m/e=270.1 (M+1).

### 4-(2-(5-bromo-2-nitrophenyl)-2-(2-(methylthio)pyrimidin-4-yl)acetyl)benzonitrile

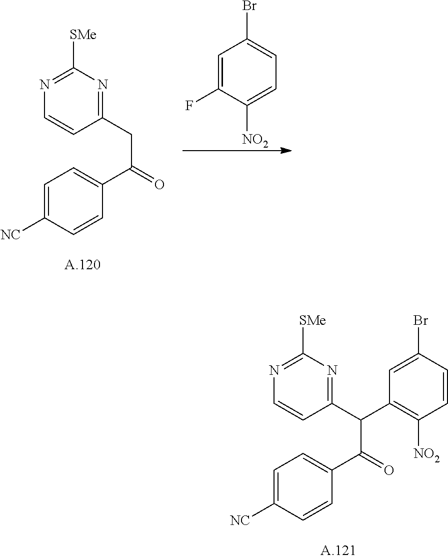

To a solution of 4-(2-(2-(methylthio)pyrimidin-4-yl)acetyl)benzonitrile A.120 (1.80 g, 6.69 mmol) in DMSO (78 mL) at 0° C. was added NaH (60% dispersion in mineral oil, 0.5348 g, 3.37 mmol) with stirring. After 5 minutes, to the mixture was added a solution of 4-bromo-2-fluoro-1-nitrobenzene (1.4709 g, 6.69 mmol) in DMSO (15 mL) with stirring, and the resulting mixture was stirred at room temperature for 18 hours. The mixture was poured into ice-water (200 mL) and extracted with ethyl acetate (300 mL×2). The combined organic layers were washed with brine (300 mL×2), dried over Na₂SO₄, filtered, and concentrated under reduced pressure to give 4-(2-(5-bromo-2-nitrophenyl)-2-(2-(methylthio)pyrimidin-4-yl)acetyl)benzonitrile A.121 (2.836 g, 90.4% yield) as an orange solid. The product was carried on in the next step: Mass Spectrum (ESI) m/e=469.0 [M+1 (⁷⁹Br)] and 471.0 [M+1 (⁸¹Br)].

### 4-(5-bromo-3-(2-(methylthio)pyrimidin-4-yl)-1H-indol-2-yl)benzonitrile

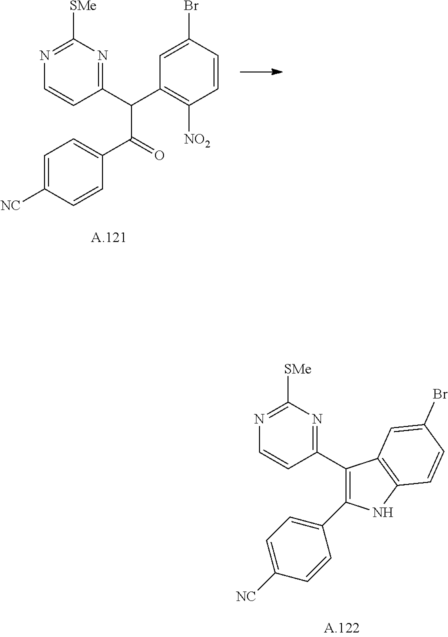

A mixture of 4-(2-(5-bromo-2-nitrophenyl)-2-(2-(methylthio)pyrimidin-4-yl)acetyl)benzonitrile A.121 (2.836 g, 6.04 mmol), Fe (1.69 g, 30.2 mmol), and acetic acid (60 mL) was heated at 95° C. with stirring for 1 hour. The mixture was cooled to room temperature, filtered, washed the solid with acetic acid (100 mL), and dried to give 4.2992 g of brown solid. The brown solid was purified by silica gel column chromatography using 30% to 50% gradient of ethyl acetate in hexane and 95% of dichloromethane in methanol as eluent to give 4-(2-(2-(methylthio)pyrimidin-4-yl)acetyl)benzonitrile A.122 (0.83 g, 32.6% yield over two steps): ¹H NMR (500 MHz, DMSO-d₆) δ ppm 12.42 (1H, s), 8.42 (1H, d, J=5.5 Hz), 8.32 (1H, d, J=1.8 Hz), 7.98 (2H, d, J=8.7 Hz), 7.76 (2H, d, J=8.2 Hz), 7.46-7.49 (1H, m), 7.40 (1H, dd, J=8.7, 1.8 Hz), 6.95 (1H, d, J=5.5 Hz), 2.39 (3H, s); Mass Spectrum (ESI) m/e=421.0 [M+1 (⁷⁹Br)] and 423.0 [M+1 (⁸¹Br)].

### 4-(3-(2-aminopyrimidin-4-yl)-5-bromo-1H-indol-2-yl)-N-tert-butylbenzamide

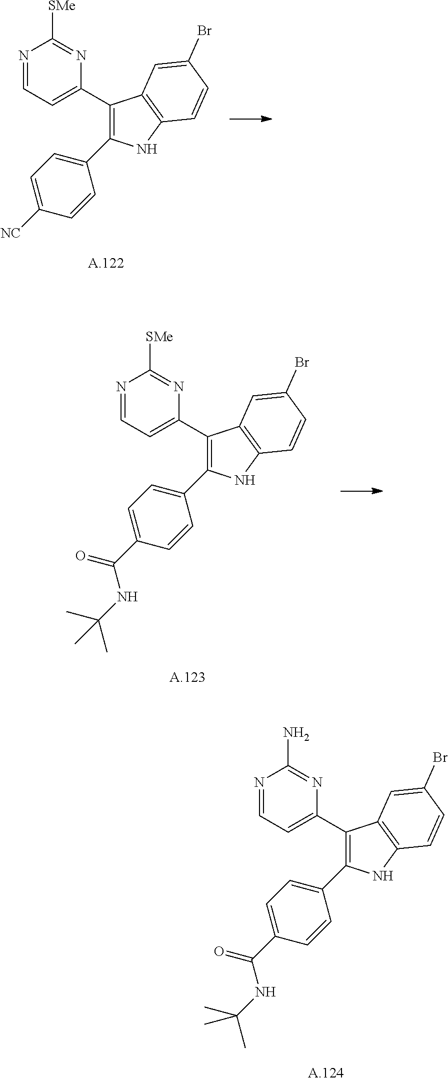

To a stirred solution of 4-(5-bromo-3-(2-(methylthio)pyrimidin-4-yl)-1H-indol-2-yl)benzonitrile A.122 (0.527 g, 1.25 mmol) in tert-butyl acetate (2.7 mL, 20.0 mmol) was added conc. H₂SO₄ (0.062 mL, 1.12 mmol) at room temperature. The mixture was stirred at 42° C. for 25 minutes. The mixture was cooled to room temperature. To the mixture was added saturated aq. NaHCO₃ to neutralize. The mixture was extracted with ethyl acetate (50 mL×2). The combined organic layers were washed with brine (50 mL×1), dried over Na₂SO₄, filtered, and concentrated under reduced pressure. The product was purified by silica gel column chromatography using 50% of ethyl acetate in hexane as eluent to give 4-(5-bromo-3-(2-(methylthio)pyrimidin-4-yl)-1H-indol-2-yl)-N-tert-butylbenzamide A.123 (0.477 g, 77% yield) as a yellow solid.

The yellow solid (0.477 g, 0.96 mmol) was dissolved in dichloromethane (20 mL) and treated with m-CPBA (0.7284 g, 2.4 mmol). The mixture was stirred at room temperature for 3 hr and concentrated under reduced pressure to give a yellow solid. The yellow solid was suspended in isopropanol (20 mL) and transferred to 150 mL of pressure vessel. To the mixture was added NH₄Cl (0.216 g, 4.04 mmol) and NH₄OH (28%, 20 mL), and the mixture was heated at 100° C. for 2 hours. The mixture was cooled to room temperature, diluted with ethyl acetate (200 mL), washed with sat'd aq. NaHCO₃ (100 mL×3), brine (100 mL×2), dried over MgSO₄, filtered, and concentrated under reduced pressure. The product was purified by silica gel column chromatography using 50% of ethyl acetate in hexane as eluent to give 4-(3-(2-aminopyrimidin-4-yl)-5-bromo-1H-indol-2-yl)-N-tert-butylbenzamide A.124 (0.237 g, 53% yield) as a yellow solid; ¹H NMR (400 MHz, DMSO-d₆) δ ppm 12.04 (1H, s), 8.36 (1H, d, J=1.9 Hz), 7.98 (1H, d, J=5.2 Hz), 7.90 (2H, d, J=8.4 Hz), 7.86 (1H, s), 7.62 (2H, d, J=8.4 Hz), 7.40 (1H, d, J=8.5 Hz), 7.32 (1H, dd, J=8.6, 1.9 Hz), 6.58 (2H, s), 6.20 (1H, d, J=5.2 Hz), 1.40 (9H, s);Mass Spectrum (ESI) m/e=464.0 [M+1 (⁷⁹Br)] and 466.1 [M+1 (⁸¹Br)].

### 4-(3-(2-aminopyrimidin-4-yl)-5-bromo-1H-indol-2-yl)benzonitrile

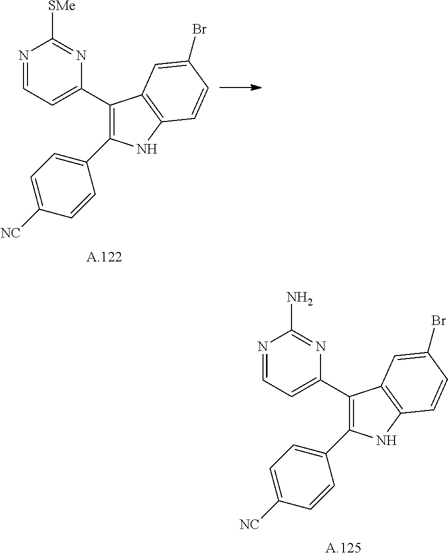

To a solution of 4-(5-bromo-3-(2-(methylthio)pyrimidin-4-yl)-1H-indol-2-yl)benzonitrile A.122 (1.493 g, 3.54 mmol) in dichloromethane (70 mL) was added m-CPBA (77%, 1.9855 g, 8.86 mmol) and the mixture was stirred at room temperature for 17 hours. The mixture was concentrated under reduced pressure to give a yellow solid. The yellow solid was suspended in isopropanol (70 mL) and transferred to 350 mL of pressure vessel. To the mixture was added NH₄Cl (0.7961 g, 14.88 mmol) and NH₄OH (28%, 70 mL) and the mixture was stirred at 100° C. for 6 hours. The mixture was diluted with ethyl acetate (200 mL) and washed with saturated aq. NaHCO₃ (100 mL×3), brine (100 mL×2), dried over MgSO₄, filtered, and concentrated under reduced pressure. The product was purified by silica gel column chromatography using 30% of dichloromethane-methanol-NH₄OH (89:9:1) in dichloromethane as eluent to give 4-(3-(2-aminopyrimidin-4-yl)-5-bromo-1H-indol-2-yl)benzonitrile A.125 (0.366 g, 26.4% overall yield over two steps) as a yellow solid: ¹H NMR (400 MHz, DMSO-d₆) δ ppm 12.16 (1H, s), 8.27 (1H, d, J=1.8 Hz), 8.05 (1H, d, J=5.2 Hz), 7.94 (2H, d, J=8.4 Hz), 7.76 (2H, d, J=8.4 Hz), 7.42 (1H, d, J=8.6 Hz), 7.34 (1H, dd, J=8.6, 1.9 Hz), 6.60 (2H, s), 6.27 (1H, d, J=5.2 Hz); Mass Spectrum (ESI) m/e=390.2 [M+1 (⁷⁹Br)] and 392.3 [M+1 (⁸¹Br)].

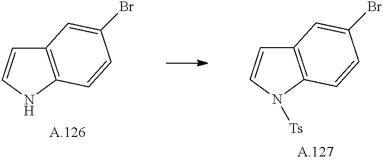

5-bromo-1-tosyl-1H-indole A.127: To a suspension of sodium hydride (60% dispersion in mineral oil, 2.448 g, 61.2 mmol) in DMF (150 mL), a solution of 5-bromo-1H-indole A.126 (10 g, 51 mmol) was added and the mixture was stirred at 0° C. for 1 hour. To the mixture was added a solution of p-TsCl (11.6695 g, 61.2 mmol) in DMF (50 mL) and the mixture was stirred at room temperature for 4.5 hours. The mixture was poured into water (500 mL) and the resulting precipitate was collected by suction filtration, washed with water (1 L), and dried to give 5-bromo-1-tosyl-1H-indole A.127 (18. g, quantitative) as a white solid: ¹H NMR (400 MHz, DMSO-d₆) δ ppm 7.78-7.93 (5H, m), 7.44-7.52 (1H, m), 7.39 (2H, d, J=8.1 Hz), 6.81 (1H, d, J=3.7 Hz), 2.32 (3H, s); Mass Spectrum (ESI) m/e=350.0 [M+1 (⁷⁹Br)] and 352.0 [M+1 (⁸¹Br)].

### 5-bromo-1-tosyl-1H-indol-3-ylboronic acid

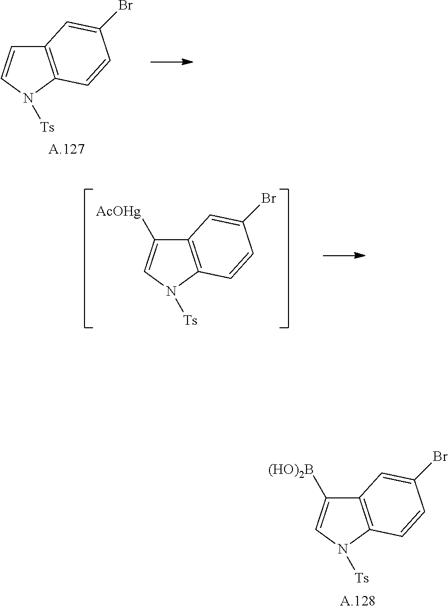

To a solution of 5-bromo-1-tosyl-1H-indole A.127 (7.6 g, 21.7 mmol) in acetic acid (145 mL) was added Hg(OAc)₂ (6.92 g, 21.7 mmol). After stirring at room temperature for 15 min, HClO₄ (5 drops) was added. The mixture was stirred at room temperature for 24 hours. The mixture was poured into water (200 mL) and the resulting precipitate was filtered, washed with water (1450 mL), and dried to give an off-white solid (12.33 g, 93.3% yield). The off white solid (5 g, 8.21 mmol) was dissolved in THF (164 mL) and to the solution at room temperature was added borane solution (1 M in THF, 41 mL, 41 mmol). After stirring at room temperature for 2 hours, to the mixture was slowly added water (49 mL). The mixture was filtered and the filtrate was concentrated under reduced pressure. The residue was extracted with ethyl acetate (150 mL×2). The combined organic layers were washed with brine (60 mL×1) and concentrated under reduced pressure to give a grey solid. The grey solid was suspended in hexane (100 mL), filtered, and washed with hexane (100 mL) to give 5-bromo-1-tosyl-1H-indol-3-ylboronic acid A.128 (3.42 g, quantitative) as a grey solid. The product as a grey solid was carried on without purification for the next step.

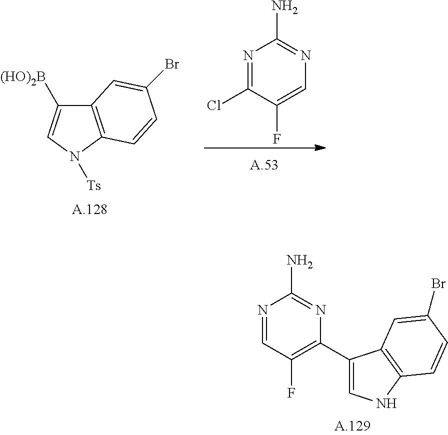

4-(5-bromo-1H-indol-3-yl)-5-fluoropyrimidin-2-amine A.129: A mixture of 5-bromo-1-tosyl-1H-indol-3-ylboronic acid (A.128) (2.62 g, 6.65 mmol), 4-chloro-5-fluoropyrimidin-2-amine A.53 (754 mg, 5.11 mmol), Pd(PPh₃)₄ (1.17 g, 1.02 mmol) and Na₂CO₃ (2M, 10 mL) in benzene (100 mL) and methanol (20 mL) was heated at 85° C. under N₂ for 24 hrs. After cooling to room temperature, the resultant mixture was diluted with ethyl acetate and filtered on a pad of Celite. The filtrates were concentrated, and the residue was charged with ethyl acetate (100 mL) and NaOH (2N, 10 mL). The mixture was heated at 90° C. for 1 hr. After cooling to room temperature, the resultant mixture was diluted with ethyl acetate and washed with water and brine, dried and concentrated. Purification of the residue by flash chromatography over silica gel, using 5:4.5:0.25 ethyl acetate-hexane-methanol, gave 4-(5-bromo-1H-indol-3-yl)-5-fluoropyrimidin-2-amine (A.129) (900.0 mg, 57%): ¹H NMR (DMSO-d₆) δ 12.04 (s, 1H), 8.87 (d, J=2.0 Hz, 1H), 8.21 (d, J=4.0 Hz, 1H), 8.15 (s, 1H), 7.47 (d, J=8.5 Hz, 1H), 7.29 (dd, J=8.5, 2.0 Hz, 1H), 6.58 (s, 2H); ms 307.0 (M+H⁺).

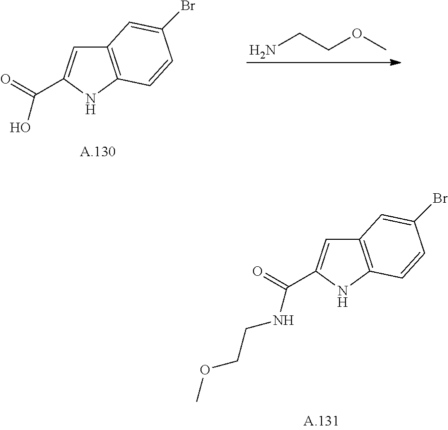

5-bromo-N-(2-methoxyethyl)-1H-indole-2-carboxamide A.131: A mixture of 5-bromo-1H-indole-2-carboxylic acid A.130 (5 g, 20.82 mmol), 2-methoxyethylamine (2.2 mL, 24.99 mmol), EDC (4.7905 g, 24.99 mmol), DMAP (4.0706 g, 33.32 mmol), and dichloromethane (100 mL) was stirred at room temperature for 18 hours. The mixture was concentrated under reduced pressure to give a yellow solid. The solid was dissolved in ethyl acetate (200 mL), washed with 2N aq. HCl (100 mL×1), water (100 mL×1), saturated aq. NaHCO₃ (100 mL×1), brine (100 mL×1), dried over Na₂SO₄, filtered, and concentrated under reduced pressure to give a yellow solid. The solid was suspended in ethyl acetate-methanol-dichloromethane (50 mL), sonicated, filtered,washed with dichloromethane. The filtrate was concentrated under reduced pressure and purified by silica gel column chromatography using 65% ethyl acetate in hexane as eluent to give 5-bromo-N-(2-methoxyethyl)-1H-indole-2-carboxamide A.131 (4.22 g, 68.2% yield) as a pale yellow solid: ¹H NMR (400 MHz, DMSO-d₆) δ ppm 11.76 (1H, s), 8.60 (1H, t, J=4.9 Hz), 7.83 (1H, d, J=1.8 Hz), 7.35-7.41 (1H, m), 7.25-7.31 (1H, m), 7.11 (1H, s), 3.40-3.51 (4H, m), 3.28 (3H, s); Mass Spectrum (ESI) m/e=297.0 [M+1 (⁷⁹Br)] and 298.9 [M+1 (⁸¹Br)].

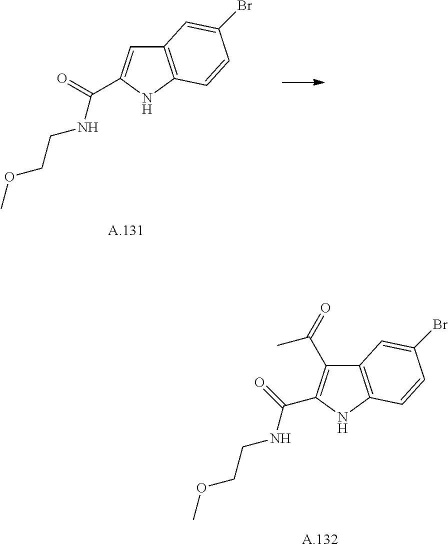

### 3-acetyl-5-bromo-N-(2-methoxyethyl)-1H-indole-2-carboxamide A.132

To a cooled solution of AlCl₃ (5.6856 g, 42.64 mol) in dichloromethane (43 mL) was added dropwise acetic anhydride (4 mL, 42.64 mmol) at 0° C. with stirring. After stirring at 0° C. for 10 minutes, to the cooled mixture was added a suspension of 5-bromo-N-(2-methoxyethyl)-1H-indole-2-carboxamide A.131 (4.22 g, 14.2 mmol) in dichloromethane (45 mL) at 0° C. and the mixture was allowed to warm to room temperature with stirring for 8 hours. The mixture was poured into ice water (250 mL) with stirring. The resulting precipitate was collected by suction filtration, washed with water (300 mL). The aq. layer was acidified with conc. HCl and extracted with ethyl acetate (350 mL×2). The precipitate and the combined organic layers were combined, washed with brine (300 mL×1), dried over Na2SO4, filtered, and concentrated under reduced pressure to give 3-acetyl-5-bromo-N-(2-methoxyethyl)-1H-indole-2-carboxamide A.132 (4.30 g, 89.3% yield): ¹H NMR (400 MHz, DMSO-d₆) δ ppm 12.75 (1 H, s), 10.28 (1H, s), 8.23 (1H, d, J=1.8 Hz), 7.49-7.53 (1H, m), 7.44 (1H, dd, J=8.6, 2.0 Hz), 3.50-3.55 (4H, m), 3.30 (3H, s), 2.65 (3H, s); Mass Spectrum (ESI) m/e=339.2 [M+1 (⁷⁹Br)] and 341.2 [M+1 (⁸¹Br)].

### 3-(2-aminopyrimidin-4-yl)-5-bromo-N-(2-methoxyethyl)-1H-indole-2-carboxamide

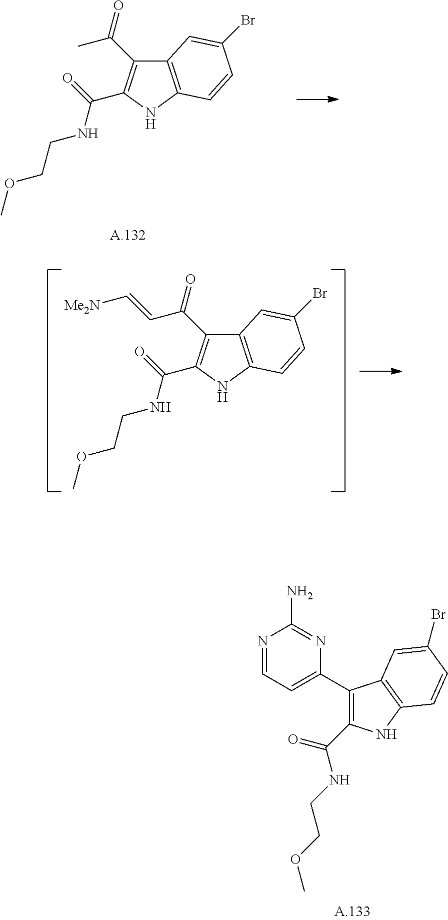

### 3-(2-aminopyrimidin-4-yl)-5-bromo-N-(2-methoxyethyl)-1H-indole-2-carboxamide A.133

A mixture of 3-acetyl-5-bromo-N-(2-methoxyethyl)-1H-indole-2-carboxamide A.132 (2 g, 5.9 mmol) in ^{t-}BuOCH(NMe₂)₂ (4.3 mL, 20.64 mmol) was stirred at 105° C. for 50 min. The mixture was cooled to room temperature. To the cooled mixture were added n-propanol (29 mL), guanidine.HCl (2.82 g, 29.48 mmol), and NaOMe/methanol (4.37 M sol., 4 mL, 17.69 mmol) and the mixture was stirred at 95° C. for 72 hours. The mixture was poured into ice water (100 mL) with stirring. The resulting precipitate was collected by suction filtration, washed with water (100 mL), and dried to give a tan solid. The product was purified by silica gel column chromatography using 95% of dichloromethane in methanol as eluent to give a tan solid. The tan solid was suspended in dichloromethane and filtered to give 3-(2-aminopyrimidin-4-yl)-5-bromo-N-(2-methoxyethyl)-1H-indole-2-carboxamide A.133 (1.644 g, 71.4% yield) as a tan solid: ¹H NMR (400 MHz, DMSO-d₆) δ ppm 12.32 (1H, s), 11.14 (1H, t, J=5.1 Hz), 8.39 (1H, d, J=5.1 Hz), 8.04 (1H, d, J=1.8 Hz), 7.48-7.54 (1H, m), 7.41 (1H, dd, J=8.8, 1.8 Hz), 7.00 (1H, d, J=5.1 Hz), 6.84 (2H, s), 3.48-3.62 (4H, m), 3.28 (3H, s); Mass Spectrum (ESI) m/e=390.2 [M+1 (⁷⁹Br)] and 392.3 [M+1 (⁸¹Br)].

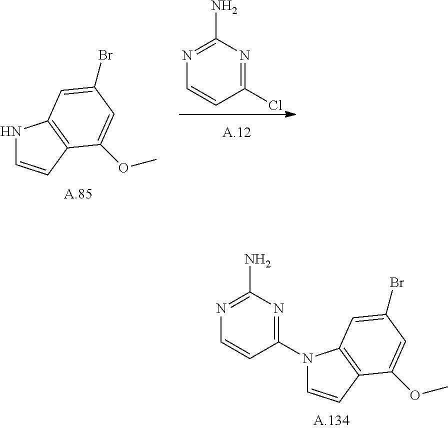

4-(6-bromo-4-methoxy-1H-indol-1-yl)pyrimidin-2-amine A.134: To a solution of 6-bromo-4-methoxy-1H-indole A.85 (2.117 g, 9.36 mmol) in DMF (46 mL) at room temperature was added Cs₂CO₃ (9.155 g, 28.1 mmol) followed by 4-chloropyrimidin-2-amine A.12 (1.456 g, 11.24 mmol) and the mixture was stirred at 80° C. for 21 hours. The mixture was poured into water (200 mL) and the aqueous layer was extracted with ethyl acetate (100 mL×2). The combined organic layers were washed with brine (100 mL×2), dried over Na₂SO₄, filtered, and concentrated under reduced pressure to give a brown solid. The product was purified by silica gel column chromatography using 65% ethyl acetate in hexane as eluent to give 4-(6-bromo-4-methoxy-1H-indol-1-yl)pyrimidin-2-amine A.134 (1.961 g, 65.6% yield) as an orange solid: ¹H NMR (400 MHz, DMSO-d₆) δ ppm 8.55 (1H, s), 8.27 (1H, d, J=5.9 Hz), 7.95 (1H, d, J=4.0 Hz), 6.89-7.04 (4H, m), 6.75 (1H, d, J=3.7 Hz), 3.92 (3H, s); Mass Spectrum (ESI) m/e=319.0 [M+1 (⁷⁹Br)] and 321.0 [M+1 (⁸¹Br)].

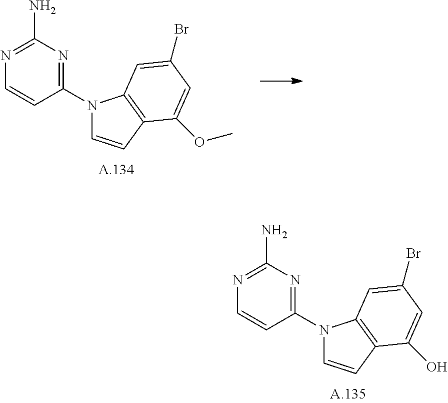

1-(2-aminopyrimidin-4-yl)-6-bromo-1H-indol-4-ol A.135: To a solution of 4-(6-bromo-4-methoxy-1H-indol-1-yl)pyrimidin-2-amine A.134 (1.868 g, 5.85 mmol) in dichloromethane (50 mL) at 0° C., was added BBr₃ (1 M sol. in dichloromethane, 29.26 mL, 29.26 mmol) dropwise. After stirring at 0° C. for 1.5 hours, the mixture was allowed to warm to room temperature. After stirring at room temperature for 48 hours, the mixture was quenched by adding water (100 mL). Then 1 N aqueous NaOH (200 mL) was added and the mixture was neutralized with 2 N aqueous HCl to pH 7.0. The mixture was extracted with dichloromethane (100 mL×3). The combined organic layers were washed with saturated aqueous NaHCO₃ (100 mL×1), brine (100 mL×2), dried over dried over Na₂SO₄, filtered, and concentrated under reduced pressure to give a brown solid. The product was purified by silica gel column chromatography using 65% to 85% gradient of ethyl acetate in hexane as eluent to give 1-(2-aminopyrimidin-4-yl)-6-bromo-1H-indol-4-ol A.135 (1.204 g, 67.4% yield) as a solid: ¹H NMR (400 MHz, DMSO-d₆) δ ppm 10.16 (1H, s), 8.37-8.40 (1H, m), 8.26 (1 H, d, J=5.9 Hz), 7.89 (1H, d, J=3.7 Hz), 6.92 (2H, s), 6.90 (1H, d, J=5.9 Hz), 6.78 (1H, dd, J=3.7, 0.7 Hz), 6.74 (1H, d, J=1.5 Hz); Mass Spectrum (ESI) m/e=305.0 [M+1 (⁷⁹Br)] and 307.0 [M+1 (⁸¹Br)].

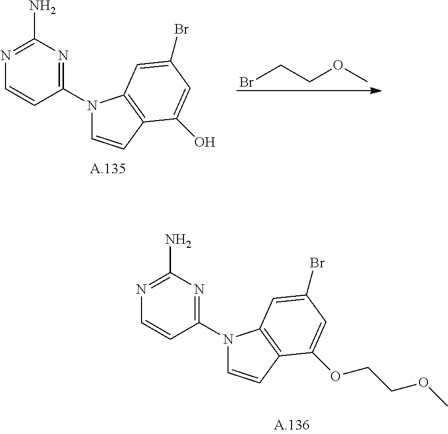

### 4-(6-bromo-4-(2-methoxyethoxy)-1H-indol-1-yl)pyrimidin-2-amine A.136

A mixture of 1-(2-aminopyrimidin-4-yl)-6-bromo-1H-indol-4-ol A.135 (0.5 g, 1.64 mmol), DMF (5 mL), Cs₂CO₃ (0.80 g, 2.46 mmol), and 1-bromo-2-methoxyethane (0.23 mL, 2.46 mmol) was stirred at room temperature for 4.5 hours. The mixture was poured into water (100 mL) with stirring. The aqueous mixture was extracted with ethyl acetate (100 mL×3). The combined organic layers were washed with brine (100 mL×1), dried over MgSO₄, filtered, and concentrated under reduced pressure and The product was purified by silica gel column chromatography using 85% ethyl acetate in hexane as eluent to give 4-(6-bromo-4-(2-methoxyethoxy)-1H-indol-1-yl)pyrimidin-2-amine A.136 (0.441 g, 74.1% yield) as a solid: ¹H NMR (400 MHz, DMSO-d₆) δ ppm 8.55 (1H, s), 8.27 (1H, d, J=5.9 Hz), 7.95 (1H, d, J=3.7 Hz), 6.96 (2H, s), 6.93 (2H, dd, J=3.7, 2.2 Hz), 6.73 (1H, d, J=3.7 Hz), 4.23-4.29 (2H, m), 3.71-3.77 (2H, m), 3.35 (3H, s); Mass Spectrum (ESI) m/e=363.0 [M+1 (⁷⁹Br)] and 365.0 [M+1 (⁸¹Br)].

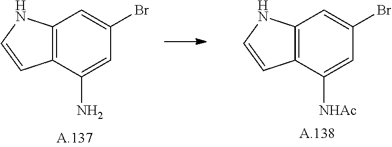

N-(6-bromo-1H-indol-4-yl)acetamide A.138: To a solution of 6-bromo-1H-indol-4-amine A.137 (0.5 g, 2.37 mmol) in Benzene (20 mL) was added acetic anhydride (0.49 mL, 5.21 mmol) and the mixture was stirred at room temperature. After stirring at room temperature for 2 hours, the mixture was concentrated under reduced pressure to give a brown solid. The brown solid was purified by silica gel column chromatography using 50% ethyl acetate in hexane as eluent to give N-(6-bromo-1H-indol-4-yl)acetamide A.138 (0.498 g, 83% yield) as a brown solid: ¹H NMR (400 MHz, DMSO-d₆) δ ppm 11.23 (1H, s), 9.70 (1H, s), 7.91 (1H, s), 7.27-7.33 (2H, m), 6.79 (1H, s), 2.16 (3H, s); Mass Spectrum (ESI) m/e=253.0 [M+1 (⁷⁹Br)] and 254.9 [M+1 (⁸¹Br)].

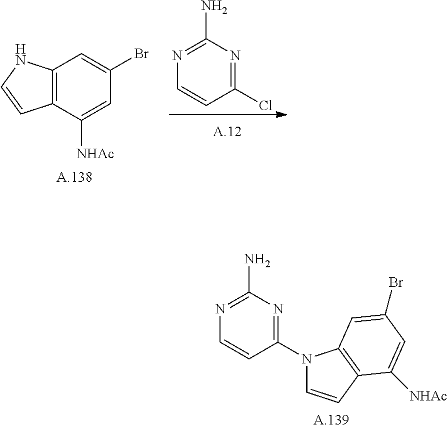

N-(1-(2-aminopyrimidin-4-yl)-6-bromo-1H-indol-4-yl)acetamide A.139: To a solution of N-(6-bromo-1H-indol-4-yl)acetamide A.138 (0.498 g, 1.97 mmol) in DMF (10 mL) at room temperature was added Cs₂CO₃ (1.9233 g, 5.9 mmol) followed by 4-chloropyrimidin-2-amine A.12 (0.306 g, 2.36 mmol) and the mixture was stirred at 80° C. for 23 hours. The mixture was poured into water (50 mL). The resulting precipitate was collected by suction filtration, washed with water (100 mL), dried, and purified by silica gel column chromatography using 30% to 100% gradient of dichloromethane-methanol-NH₄OH (89:9:1) in dichloromethane as eluent to give N-(1-(2-aminopyrimidin-4-yl)-6-bromo-1H-indol-4-yl)acetamide A.139 (0.384 g, 56.3% yield) as a brown solid: ¹H NMR (400 MHz, DMSO-d₆) δ ppm 9.83 (1H, s), 8.68 (1 H, d, J=1.1 Hz), 8.28 (1H, d, J=5.5 Hz), 8.07 (1H, d, J=1.1 Hz), 8.03 (1H, d, J=4.0 Hz), 7.12 (1H, d, J=3.7 Hz), 6.98 (2H, s), 6.92 (1H, d, J=5.5 Hz), 2.17 (3H, s); Mass Spectrum (ESI) m/e=345.9 [M+1 (⁷⁹Br)] and 347.8 [M+1 (⁸¹Br)].

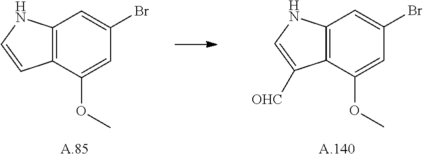

### 6-bromo-4-methoxy-1H-indole-3-carbaldehyde A.140

To a solution of 6-bromo-4-methoxy-1H-indole A.85 (2.0 g, 8.85 mmol) in DMF (9 mL) was added (chloromethylene)dimethylammonium chloride (1.699 g, 13.27 mmol) and the mixture was stirred at room temperature for 24 hours. To the mixture was added water (50 mL) and 10 N aqueous NaOH (10 mL) and the mixture was heated at reflux for 1 hour. The mixture was cooled to 0° C. The resulting precipitate was collected by filtration to give 6-bromo-4-methoxy-1H-indole-3-carbaldehyde A.140 (1.392 g, 61.9% yield) as a brown solid: ¹H NMR (400 MHz, DMSO-d₆) δ ppm 12.26 (1H, s), 10.26 (1H, s), 8.05 (1H, s), 7.30 (1H, d, J=1.5 Hz), 6.89 (1H, d, J=1.1 Hz), 3.95 (3H, s); Mass Spectrum (ESI) m/e=253.9 [M+1 (⁷⁹Br)] and 255.9 [M+1 (⁸¹Br)].

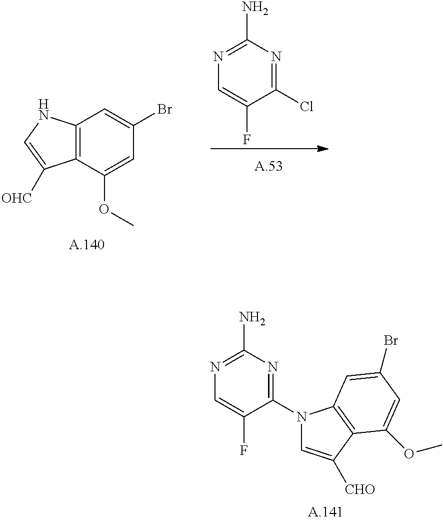

1-(2-amino-5-fluoropyrimidin-4-yl)-6-bromo-4-methoxy-1H-indole-3-carbaldehyde A.141: To a solution of 6-bromo-4-methoxy-1H-indole-3-carbaldehyde A.140 (0.65 g, 2.56 mmol) in DMF (13 mL) at room temperature was added Cs₂CO₃ (2.50 g, 7.67 mmol) followed by 4-chloro-5-fluoropyrimidin-2-amine A.53 (0.453 g, 3.07 mmol) and the mixture was stirred at 80° C. for 6 hours. The mixture was concentrated under reduced pressure and purified by silica gel column chromatography using 0% to 100% gradient of dichloromethane-methanol-NH₄OH (89:9:1) in dichloromethane as eluent to give 1-(2-amino-5-fluoropyrimidin-4-yl)-6-bromo-4-methoxy-1H-indole-3-carbaldehyde A.141 (0.319 g, 34.2% yield) as a yellow solid:

¹H NMR (500 MHz, DMSO-d₆) δ ppm 10.39 (1H, s), 8.56 (1H, d, J=3.7 Hz), 8.32 (1H, d, J=2.7 Hz), 7.95 (1H, s), 7.13 (2H, s), 7.12 (1H, d, J=1.4 Hz), 4.01 (3H, s); Mass Spectrum (ESI) m/e=365.0 [M+1 (⁷⁹Br)] and 367.0 [M+1 (⁸¹Br)].

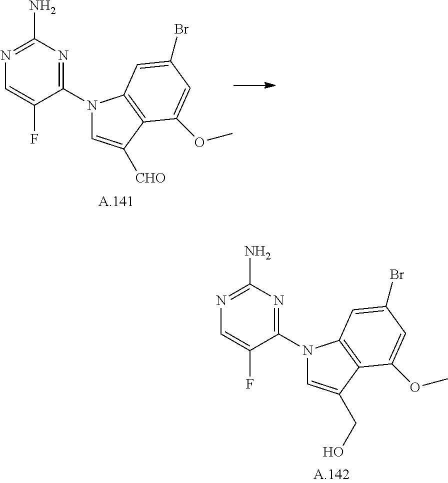

### (1-(2-amino-5-fluoropyrimidin-4-yl)-6-bromo-4-methoxy-1H-indol-3-yl)methanol A.142

To a solution of 1-(2-amino-5-fluoropyrimidin-4-yl)-6-bromo-4-methoxy-1H-indole-3-carbaldehyde A.141 (0.319 g, 0.874 mmol) in DMF-methanol (1:1, 10 mL) was added NaBH₄ (0.1432 g, 3.78 mmol) and the mixture was stirred at room temperature for 1 hour. To the mixture was added water (50 mL) with stirring. The resulting precipitate was collected by filtration, washed with water (100 mL), and dried to give (1-(2-amino-5-fluoropyrimidin-4-yl)-6-bromo-4-methoxy-1H-indol-3-yl)methanol A.142 (0.22 g, 68.4%) as a tan solid: ¹H NMR (500 MHz, DMSO-d₆) δ ppm 8.42 (1H, d, J=4.6 Hz), 8.11 (1H, s), 7.50-7.55 (1H, m), 6.96 (2H, s), 6.89 (1H, d, J=1.4 Hz), 5.02 (1H, t, J=5.5 Hz), 4.78 (2H, dd, J=5.5, 1.4 Hz), 3.89 (3 H, s); Mass Spectrum (ESI) m/e=367.0 [M+1 (⁷⁹Br)] and 369.0 [M+1 (⁸¹Br)].

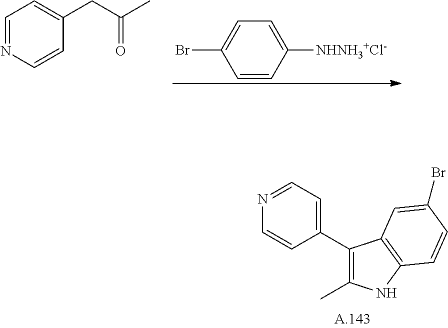

### 5-bromo-2-methyl-3-(pyridin-4-yl)-1H-indole A.143

4-pyridylacetone (2.69 g, 0.02 mmol)) and 4-bromophenylhydrazine hydrochloride (4.66 g, 0.021 mmol) in 20 ml of n-propanol were heated. Conc. Sulfuric acid (2.92 g) was added slowly to the mixture. The reaction was heated to reflux for 14 hr. After cooling, the solution was carefully quenched into sodium bicarbonate solution. Solids were collected by filtration and washed with water to give 5-bromo-2-methyl-3-(pyridin-4-yl)-1H-indole (5.65 g, 99% yield on crude, ˜78% pure by HPLC). A 1 g portion was suspended in hot ethyl acetate (40 ml). Filtration of the hot suspension afforded 0.3 g of analytically pure 5-bromo-2-methyl-3-(pyridin-4-yl)-1H-indole A.143: Mp 248.1° C.; ¹H NMR (400 MHz, DMSO-d6) δ 11.66 (s, 1H); 8.602 (dd, J=4.4, 1.6 Hz, 1H); 7.737 (d, J=2Hz, 1H); 7.491 (dd, J=4.8, 1.6 Hz, 1H); 7.350 (d, J=8.4 Hz, 1H); 7.228 (dd, J=8.4, 2 Hz, 1H); 2.529 (s, 3H) ppm; Mass Spectrum (ESI) m/e=287 [M+1 (⁷⁹Br)] and 289 [M+1 (⁸¹Br)]; CHN. Found: 58.63% C, 3.99% H, 9.75% N, 27.70% Br Theory 58.56% C, 3.86% H, 9.76% N, 27.83% Br.

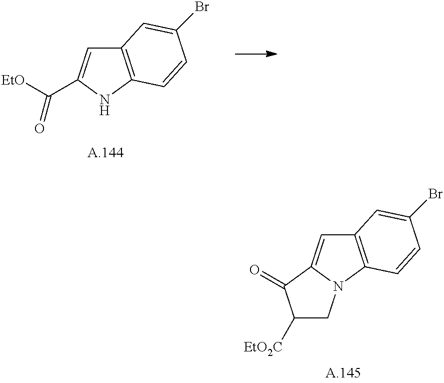

ethyl 7-bromo-1-oxo-2,3-dihydro-1H-pyrrolo[1,2-a]indole-2-carboxylate A.145: A mixture of ethyl 5-bromo-1H-indole-2-carboxylate A.144 (9.94 g, 37.1 mmol), benzene (74 mL), and ^{t-}BuOK (1 M sol. In ^{t-}BuOH, 37.1 mL, 37.1 mmol) was stirred at room temperature for 5 minutes. To the mixture was added ethyl acrylate (4.02 mL, 37.09 mmol) and the mixture was heated under reflux for 24 hours. Water (100 mL) was added to the mixture with stirring. The mixture was acidified to pH 2.0 with 2 N aqueous HCl (20 mL) and diluted with ethyl acetate (100 mL). The organic layer was separated, washed with water (100 mL×3), dried over Na₂SO₄, filtered, and concentrated under reduced pressure to give ethyl 7-bromo-1-oxo-2,3-dihydro-1H-pyrrolo[1,2-a]indole-2-carboxylate A.145 (13.71 g on crude) as a yellow oil. The product was carried on to the next step: Mass Spectrum (ESI) m/e=322.0 [M+1 (⁷⁹Br)] and 324.0 [M+1 (⁸¹Br)].

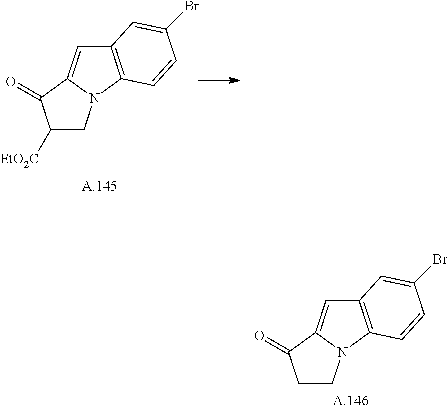

7-bromo-2,3-dihydropyrrolo[1,2-a]indol-1-one A.146: A solution of 7-bromo-1-oxo-2,3-dihydro-1H-pyrrolo[1,2-a]indole-2-carboxylate A.145 (37.09 mmol) in 95% of aqueous acetic acid (40 mL) was heated at reflux with stirring for 27 hours. Water (150 mL) was added to the mixture with stirring. The resulting precipitate was collected by filtration and washed with water (300 mL) and air-dried to give a brown solid. The brown solid was dissolved in ethyl acetate (500 mL), dried over Na2SO4, filtered, and concentrated under reduced pressure to give a brown solid. The brown solid was purified by silica gel column chromatography using 30% of ethyl acetate in hexane as eluent to give 7-bromo-2,3-dihydropyrrolo[1,2-a]indol-1-one A.146 (4.27 g, 45% overall yield over two steps) as a yellow solid: ¹H NMR (400 MHz, DMSO-d₆) δ ppm 7.98 (1H, d, J=1.8 Hz), 7.62 (1H, d, J=8.8 Hz), 7.46 (1H, dd, J=9.0, 2.0 Hz), 6.91 (1H, s), 4.46 (2H, t, J=6.2 Hz), 3.17-3.22 (2H, m); Mass Spectrum (ESI) m/e=250.0 [M+1 (⁷⁹Br)] and 251.9 [M+1 (⁸¹Br)].

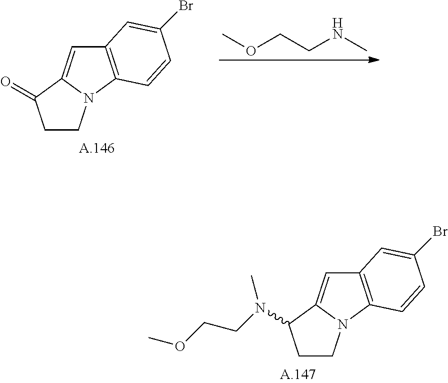

7-bromo-N-(2-methoxyethyl)-N-methyl-2,3-dihydro-1H-pyrrolo[1,2-a]indol-1-amine A.147: To a solution of 7-bromo-2,3-dihydropyrrolo[1,2-a]indol-1-one A.146 (1.5 g, 5.998 mmol) in methanol-acetic acid (20:1, 30 mL), DMF (5 mL), and DCE (8 mL), was added 2-methoxy-N-methylethanamine (1.3 mL, 11.996 mmol) followed by NaBH₃CN (1.8845 g, 29.99 mmol) and then the mixture was stirred at 80° C. for 25 hours. The mixture was poured into saturated aqueous NaHCO₃ (250 mL) with stirring and basified to pH 10.0 with 2 N aqueous NaOH (40 mL). The mixture was extracted with dichloromethane (100 mL×1). The combined organic layers were washed with water (100 mL×2), brine (100 mL×2), dried over Na₂SO₄, filtered, and concentrated under reduced pressure to give a yellow oil. The product was purified by silica gel column chromatography using 0% to 100% gradient of dichloromethane-methanol-NH₄OH (89:9:1) in dichloromethane as eluent to give 7-bromo-N-(2-methoxyethyl)-N-methyl-2,3-dihydro-1H-pyrrolo[1,2-a]indol-1-amine A.147 (1.655 g, 85.4% yield) as a brown syrup: Mass Spectrum (ESI) m/e=323.0 [M+1 (⁷⁹Br)] and 325.1 [M+1 (⁸¹Br)].

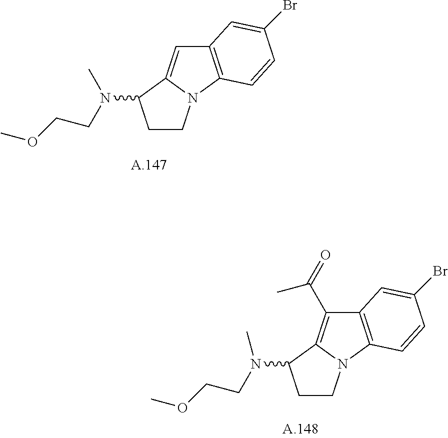

1-(7-bromo-1-((2-methoxyethyl)(methyl)amino)-2,3-dihydro-1H-pyrrolo[1,2-a]indol-9-yl)ethanone A.148: To a cooled solution of AlCl₃ (1.95 g, 14.54 mmol) in dichloromethane (14 mL) was added dropwise acetic anhydride (1.37 mL, 14.54 mmol) at 0° C. with stirring. The mixture was stirred at 0° C. for 10 minutes. To the cooed mixture was added a solution of 7-bromo-N-(2-methoxyethyl)-N-methyl-2,3-dihydro-1H-pyrrolo[1,2-a]indol-1-amine A.147 (1.567 g, 4.85 mmol) in dichloromethane (16 mL). The mixture was allowed to warm to room temperature and stirred at room temperature for 48 hours. The mixture was poured into ice water (100 mL) with stirring and extracted with dichloromethane (100 mL×2). The combined organic layers were washed with saturated aqueous NaHCO₃ (100 mL×1), brine (100 mL×1), dried over Na₂SO₄, filtered, and concentrated under reduced pressure. The product was purified by silica gel column chromatography using 10% to 20% gradient of dichloromethane-methanol-NH₄OH (89:9:1) in dichloromethane as eluent to give 1-(7-bromo-1-((2-methoxyethyl)(methyl)amino)-2,3-dihydro-1H-pyrrolo[1,2-a]indol-9-yl)ethanone A.148 (1.46 g, 82.4% yield) as a dark brown syrup: ¹H NMR (400 MHz, DMSO-d₆) δ ppm 8.38 (1H, d, J=1.2 Hz), 7.41-7.46 (1H, m), 7.35 (1H, dd, J=8.6, 2.0 Hz), 4.93 (1H, dd, J=8.4, 1.8 Hz), 4.07-4.30 (2H, m), 3.40 (2H, t, J=5.7 Hz), 3.18 (3H, s), 2.64-2.77 (2H, m), 2.63 (3H, s), 2.52-2.61 (1H, m), 2.39-2.48 (1H, m), 2.10 (3H, s); Mass Spectrum (ESI) m/e=365.0 [M+1 (⁷⁹Br)] and 367.1 [M+1 (8′Br)].

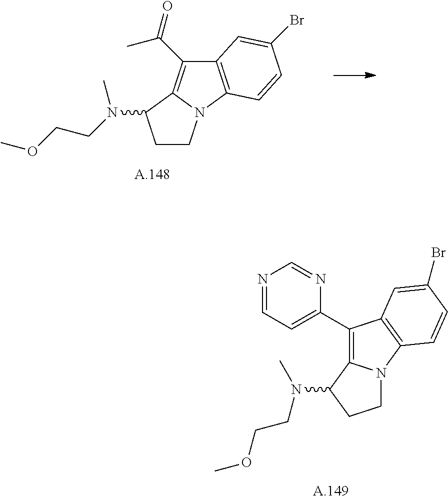

7-bromo-N-(2-methoxyethyl)-N-methyl-9-(pyrimidin-4-yl)-2,3-dihydro-1H-pyrrolo[1,2-a]indol-1-amine A.149: A mixture of 1-(7-bromo-1-((2-methoxyethyl)(methyl)amino)-2,3-dihydro-1H-pyrrolo[1,2-a]indol-9-yl)ethanone A.148 (1 g, 2.74 mmol) in ^{t-}BuOCH(NMe₂)₂ (1.2 mL, 5.48 mmol) was stirred at 105° C. After stirring at 105° C. for 2.5 hours, the mixture was cooled to room temperature. To the cooled mixture were added ⁿ⁻PrOH (26 mL), formamidine acetate (1.4251 g, 13.7 mmol), and NaOMe in methanol (4.37 M sol., 1.9 mL, 8.303 mmol) and the mixture was stirred at 95° C. for 48 hours. The mixture was poured into ice water (100 mL) with stirring. The aqueous mixture was extracted with dichloromethane (100 mL×3). The combined organic layers were washed with brine (100 mL×1), dried over Na₂SO₄, filtered, and concentrated under reduced pressure. The product was purified by silica gel column chromatography using 0% to 100% gradient of dichloromethane-methanol-NH₄OH (89:9:1) in dichloromethane as eluent to give 7-bromo-N-(2-methoxyethyl)-N-methyl-9-(pyrimidin-4-yl)-2,3-dihydro-1H-pyrrolo[1,2-a]indol-1-amine A.149 (0.635 g, 57.8% yield) as a dark red syrup: ¹H NMR (400 MHz, DMSO-d₆) δ ppm 9.13 (1H, d, J=0.8 Hz), 8.88 (1H, d, J=2.0 Hz), 8.59 (1H, d, J=5.9 Hz), 8.28 (1H, dd, J=5.7, 1.4 Hz), 7.41-7.47 (1H, m), 7.32-7.38 (1H, m), 4.93 (1H, dd, J=8.4, 2.9 Hz), 4.13-4.28 (2H, m), 3.45 (2H, t, J=5.3 Hz), 3.25 (3H, s), 2.68-2.80 (2H, m), 2.52-2.65 (2H, m), 2.11 (3H, s); Mass Spectrum (ESI) m/e=401.0 [M+1 (⁷⁹Br)] and 403.1 [M+1 (⁸¹Br)].

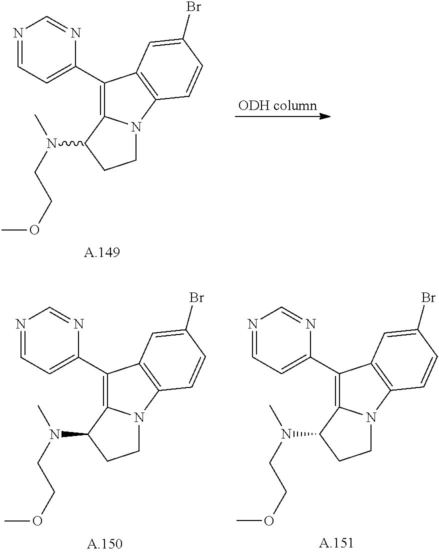

The racemic mixture A.149 was separated on a Chiralcel OD-H column using 3% isocratic of isopropanol in hexane as eluent to give two separated isomers. The stereochemistry of the two isomers was arbitrarily assigned the first peak A.150 as R-isomer and the second peak A.151 as S-isomer on OD-H column.

### (1R*)-7-bromo-N-(2-methoxyethyl)-N-methyl-9-(pyrimidin-4-yl)-2,3-dihydro-1H-pyrrolo[1,2-a]indol-1-amine A.150

1H NMR (500 MHz, DMSO-d₆) δ ppm 9.13 (1H, s), 8.88 (1H, d, J=1.7 Hz), 8.60 (1H, d, J=5.4 Hz), 8.29 (1H, d, J=5.6 Hz), 7.45 (1H, d, J=8.8 Hz), 7.36 (1H, dd, J=8.6, 2.0 Hz), 4.93 (1H, dd, J=8.3, 2.7 Hz), 4.13-4.28 (2H, m), 3.46 (2H, t, J=5.5 Hz), 3.25 (3H, s), 2.70-2.80 (2H, m), 2.52-2.66 (2H, m), 2.11 (3H, s)

### (1S*)-7-bromo-N-(2-methoxyethyl)-N-methyl-9-(pyrimidin-4-yl)-2,3-dihydro-1H-pyrrolo[1,2-a]indol-1-amine A.151

1H NMR (500 MHz, DMSO-d₆) δ ppm 9.13 (1H, s), 8.88 (1H, d, J=1.7 Hz), 8.60 (1H, d, J=5.4 Hz), 8.29 (1H, d, J=5.6 Hz), 7.45 (1H, d, J=8.8 Hz), 7.36 (1H, dd, J=8.6, 2.0 Hz), 4.93 (1H, dd, J=8.3, 2.7 Hz), 4.13-4.28 (2H, m), 3.46 (2H, t, J=5.5 Hz), 3.25 (3H, s), 2.70-2.80 (2H, m), 2.52-2.66 (2H, m), 2.11 (3H, s)

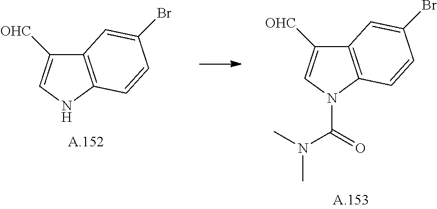

5-bromo-3-formyl-N,N-dimethyl-1H-indole-1-carboxamide A.153: To a 5-bromo-1H-indole-3-carbaldehyde A.152 (2.69 g, 12.0 mmol) in DMF (34 mL) was added NaH (60% dispersion in mineral oil, 0.72 g, 18 mmol) at 0° C. and the mixture was allowed to warm to room temperature over 10 minutes. To the mixture was added dimethylcarbamic chloride (1.33 mL, 14.4 mmol) and the mixture was stirred at room temperature for 6 hours. To the mixture was added water (100 mL). The mixture was extracted with dichloromethane (100 mL×2). The combined organic layers were washed with brine (100 mL×1), dried over Na₂SO₄, filtered, and concentrated under reduced pressure to give an orange oil. The product was purified by silica gel column chromatography using 0% to 100% gradient of ethyl acetate in hexane as eluent to give 5-bromo-3-formyl-N,N-dimethyl-1H-indole-1-carboxamide A.153 (3.50 g, 98.6% yield) as a yellow syrup: ¹H NMR (500 MHz, DMSO-d₆) δ ppm 10.01 (1 H, s), 8.64 (1H, s), 8.27 (1H, s), 7.50-7.69 (2H, m), 3.05 (6H, s); Mass Spectrum (ESI) m/e=295.0 [M+1 (⁷⁹Br)] and 297.0 [M+1 (⁸¹Br)].

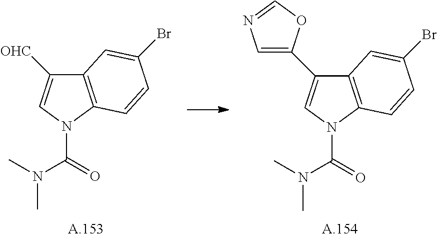

### 5-bromo-N,N-dimethyl-3-(oxazol-5-yl)-1H-indole-1-carboxamide A.154

To a solution of 5-bromo-3-formyl-N,N-dimethyl-1H-indole-1-carboxamide A.153 (3.50 g, 11.84 mmol) in DME (150 mL) was added TosMIC (2.312 g, 11.84 mmol) followed by DBU (1.95 mL, 13.03 mmol) and the mixture was stirred at 80° C. for 2 hours. The mixture was cooled to room temperature and concentrated under reduced pressure to give a dark red syrup. The product was purified by silica gel column chromatography using % ethyl acetate in hexane as eluent to give 5-bromo-N,N-dimethyl-3-(oxazol-5-yl)-1H-indole-1-carboxamide A.154 (2.755 g, 69.6% yield on crude) as a red syrup: ¹H NMR (500 MHz, DMSO-d₆) δ ppm 8.43-8.47 (1H, m), 8.11 (2H, s), 7.70-7.73 (1H, m), 7.64 (1H, d, J=8.8 Hz), 7.50 (1H, dd, J=8.8, 1.0 Hz), 3.05 (6H, s); Mass Spectrum (ESI) m/e=334.0 [M+1 (⁷⁹Br)] and 336.0 [M+1 (⁸¹Br)].

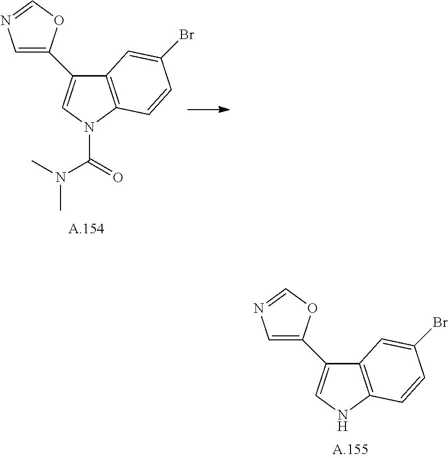

5-bromo-3-(oxazol-5-yl)-1H-indole A.155: To a solution of 5-bromo-N,N-dimethyl-3-(oxazol-5-yl)-1H-indole-1-carboxamide A.154 (2.755 g, 8.24 mmol) in methanol (100 mL) at 0° C. was added 1N aqueous NaOH (22 mL) and the mixture was stirred at 0° C. for 2 hours. The mixture was partitioned between dichloromethane (100 mL) and saturated aqueous NH₄Cl (100 mL). The organic layer was separated, dried over MgSO₄, filtered, and concentrated under reduced pressure. The product was purified by silica gel column chromatography using 0% to 100% gradient of ethyl acetate in hexane as eluent to give 5-bromo-3-(oxazol-5-yl)-1H-indole A.155 (0.41 g, 18.9% yield) as a light yellow solid: ¹H NMR (500 MHz, DMSO-d₆) δ ppm 11.78 (1H, s), 8.27-8.38 (1H, m), 8.01 (1H, d, J=1.5 Hz), 7.87 (1H, s), 7.50 (1H, s), 7.45 (1H, d, J=8.6 Hz), 7.32 (1H, dd, J=8.6, 1.2 Hz); Mass Spectrum (ESI) m/e=262.9 [M+1 (⁷⁹Br)] and 265.0 [M+1 (⁸¹Br)].

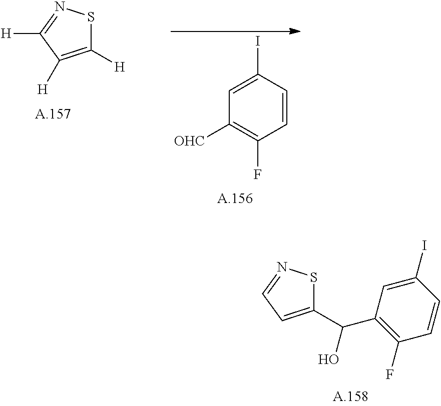

(2-fluoro-5-iodophenyl)(isothiazol-5-yl)methanol A.158: To a solution of isothiazole (A.157) (3.0 g, 35.3 mmol) in 50 mL THF at −78° C. under argon was added n-butyllithium (1.6 M in hexane, 22.1 mL, 35.3 mmol) dropwise over 10 minutes. The mixture stirred at −78° C. for 1.5 hours. A solution of 2-fluoro-5-iodobenzaldehyde (A.156)(7.35 g, 29.4 mmol) in 30 mL THF was added dropwise over 15 minutes, and the reaction stirred an additional 2 hours at −78° C. Water was added to quench the reaction, and the mixture warmed to 0° C. The solution was neutralized with 2 N HCl to pH 7, partitioned between water and dichloromethane, and the aqueous layer further extracted with dichloromethane. The combined organic extracts were washed with brine, dried over MgSO₄ and condensed. Purification by flash chromatography (0-15-100% ethyl acetate in hexane) gave 7.19 g (2-fluoro-5-iodophenyl)(isothiazol-5-yl)methanol A.158. 1H NMR (500 MHz, CHLOROFORM-d) δ ppm 8.30 (1H, s), 7.76 (1H, dd, J=6.8, 2.2 Hz), 7.56 (1H, ddd, J=8.6, 5.0, 2.3 Hz), 6.97 (1H, s), 6.79 (1H, dd, J=9.8, 8.8 Hz), 6.37 (1H, s)

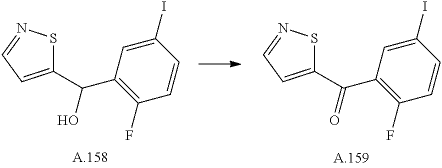

(2-fluoro-5-iodophenyl)(isothiazol-5-yl)methanone A.159: (2-fluoro-5-iodophenyl)(isothiazol-5-yl)methanol A.158 (7.19 g, 21.4 mmol) was stirred in 28 mL acetic acid at room temperature as CrO₃ (4.28 g, 42.9 mmol) was added portionwise. The resulting solution was heated to 100° C. for 15 minutes. After cooling to room temperature, the mixture was partitioned between water and dichloromethane, and the aqueous layer was further extracted with dichloromethane. The combined organic extracts were washed with 2 M aq. NaOH and water, dried over MgSO₄ and condensed to give 5.67 g (2-fluoro-5-iodophenyl)(isothiazol-5-yl)methanone A.159. 1H NMR (500 MHz, CHLOROFORM-d) δ ppm 8.60 (1H, d, J=1.2 Hz), 7.94 (1H, dd, J=6.2, 2.1 Hz), 7.84-7.92 (1H, m), 7.57 (1H, s), 7.01 (1H, t, J=9.0 Hz)

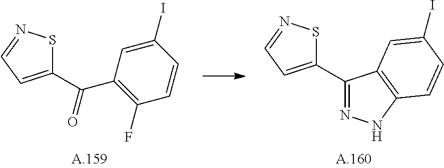

5-iodo-3-(isothiazol-5-yl)-1H-indazole A.160: To a stirring solution of (2-fluoro-5-iodophenyl)(isothiazol-5-yl)methanone A.159 (5.67 g, 17 mmol) in DMSO (17 mL) was added anhydrous hydrazine (2.14 mL, 68 mmol), and the resulting mixture was stirred at 80° C. The heat was removed after 8 hours, water was added and the solution cooled to room temperature. The mixture was extracted into ethyl acetate, dried over MgSO₄ and condensed. Purification by flash chromatography (0-10-100% ethyl acetate in hexane) gave 0.714 g 5-iodo-3-(isothiazol-5-yl)-1H-indazole A.160. 1H NMR (500 MHz, DMSO-d₆) δ ppm 13.76 (1H, s), 8.66 (1H, s), 8.52 (1H, s), 8.13 (1H, s), 7.72 (1H, d, J=8.8 Hz), 7.51 (1H, d, J=8.8 Hz)

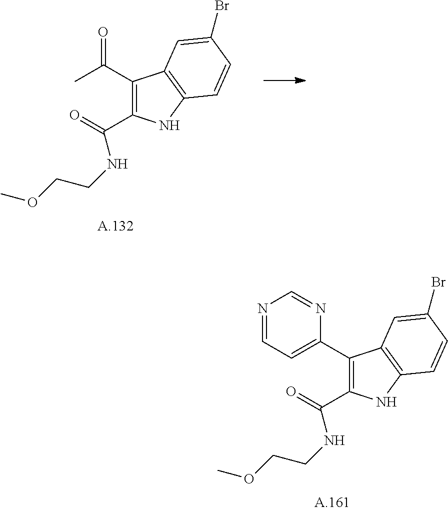

### 5-bromo-N-(2-methoxyethyl)-3-(pyrimidin-4-yl)-1H-indole-2-carboxamide A.161

This compound was prepared from compound A.132 by the procedure used to prepare compound A.149.

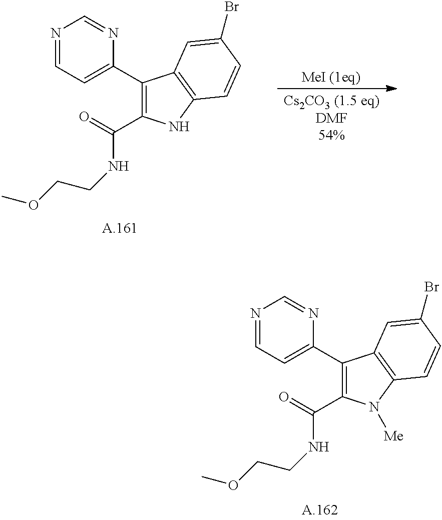

### 5-bromo-N-(2-methoxyethyl)-1-methyl-3-(pyrimidin-4-yl)-1H-indole-2-carboxamide (A.162)

To a stirred solution of A.161 (500 mg, 1.3 mmol) and Cs₂CO₃ (387 mg, 2.0 mmol) in 5 mL of DMF was added MeI (190 mg, 1.3 mmol) at room temperature and the mixture was stirred. After stirring overnight, the mixture was partitioned between DCM and water. The organic layer was washed with brine, dried over MgSO₄, filtered, and concentrated under reduced pressure. The crude product was purified by silica gel column chromatography using 0 to 100% gradient of DCM-MeOH—NH₄OH (89:9:1) in DCM to give 5-bromo-N-(2-methoxyethyl)-1-methyl-3-(pyrimidin-4-yl)-1H-indole-2-carboxamide A.162 (273 mg, 54% yield): Mass Spectrum (ESI) m/e=389.0 [M+1 (⁷⁹Br)] and 391.0 [M+1 (⁸¹Br)].

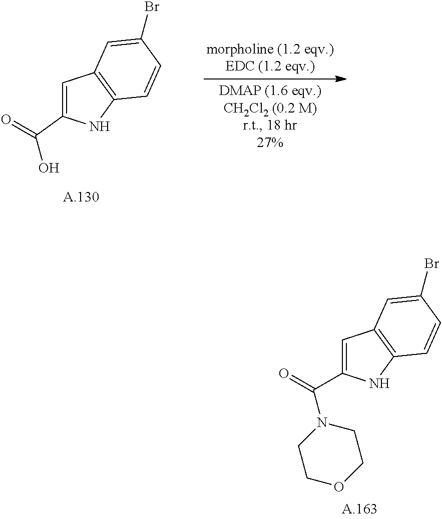

(5-bromo-1H-indol-2-yl)(morpholino)methanone A.163: A mixture of A.130 (3 g, 12.5 mmol), morpholine (1.31 mL, 15 mmol), DMAP (2.44 g, 20 mmol), and EDC (2.87 g, 15 mmol) was stirred in DCM (60 mL) at room temperature. After 18 hr, the mixture was concentrated under reduced pressure to give a yellow solid. The yellow solid was dissolved in EtOAc (200 mL). The mixture was washed with 2 N aq. HCl (100 mL), water (100 mL), sat. aq. NaHCO₃, dried over Na₂SO₄, filtered, and concentrated under reduced pressure to give a yellow solid (1.42 g). The yellow solid was triturated with acetone-EtOAc and the resulting solid was collected by filtration to give the desired product A.163 (1.034 g, 27% yield) as a yellow solid: 1H NMR (500 MHz, CHLOROFORM-d) δ ppm 9.32 (1H, s), 7.80 (1H, d, J=1.7 Hz), 7.40 (1H, dd, J=8.6, 1.7 Hz), 7.33 (1H, d, J=8.6), 6.72 (1H, dd, J=2.0, 0.7 Hz), 3.96 (4H, br. s.), 3.78-3.85 (4H, m); Mass Spectrum (ESI) m/e=309.0 [M+1 (⁷⁹Br)] and 311.0 [M+1 (⁸¹Br)].

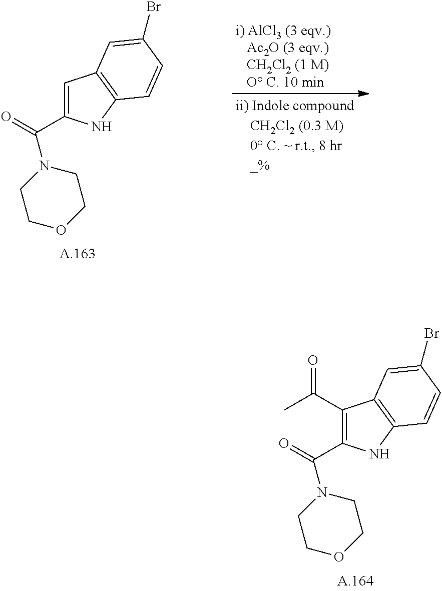

1-(5-bromo-2-(morpholine-4-carbonyl)-1H-indol-3-yl)ethanone A.164 was prepared from A.163 by the procedure used to prepare compound A.132: 1H NMR (500 MHz, CHLOROFORM-d) δ ppm 10.08 (1H, s), 8.12 (1H, d, J=1.7 Hz), 7.36 (1H, dd, J=8.8, 1.5 Hz), 7.21 (1H, d, J=8.8 Hz), 3.67 (3H, s), 2.55-2.71 (8H, m); Mass Spectrum (ESI) m/e=351.0 [M+1 (⁷⁹Br)] and 353.0 [M+1 (⁸¹Br)].

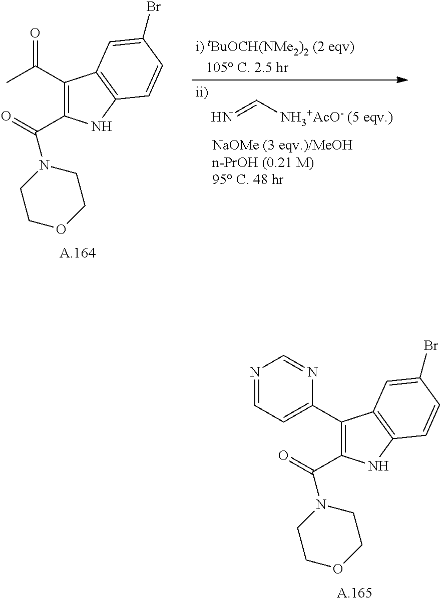

(5-bromo-3-(pyrimidin-4-yl)-1H-indol-2-yl)(morpholino)methanone A.165 was prepared from compound A.164 by the procedure used to prepare compound A.149. 1H NMR (500 MHz, CHLOROFORM-d) δ ppm 9.95, (1H, s), 9.26 (1H, s), 8.76 (1H, d, J=5.6 Hz), 8.33 (1 H, s), 7.63 (1H, dd, J=5.4, 1.2 Hz), 7.44 (1H, dd, J=8.6, 1.7 Hz), 7.37 (1H, d, J=8.6 Hz), 3.78 (m, 4H), 3.45 (m, 2H), 3.26 (m, 2H); Mass Spectrum (ESI) m/e=387.0 [M+1 (⁷⁹Br)] and 389.0 [M+1 (⁸¹Br)].

### N-cyclopropyl-5-((R)-3-hydroxy-3-(4-methyl-5H-pyrrol-2-yl)but-1-ynyl)-3-(pyrimidin-4-yl)-1H-indole-2-carboxamide

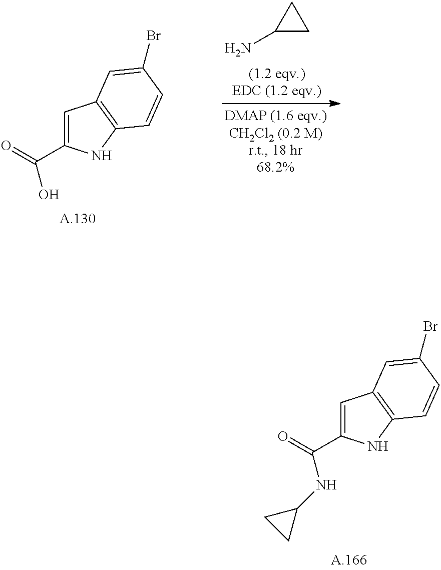

5-bromo-N-cyclopropyl-1H-indole-2-carboxamide A.166 was prepared from A.130 by the procedure used to prepare compound A.163:: 1H NMR (500 MHz, DMSO-d₆) δ ppm 11.77 (1 H, s), 8.54 (1H, d, J=3.7 Hz), 7.81 (1H, s), 7.38 (1H, d, J=8.6 Hz), 7.27 (1H, d, J=8.8 Hz), 7.07 (1H, s), 2.86 (1H, qd, J=7.4, 3.9 Hz), 0.73 (2H, td, J=6.9, 5.0 Hz), 0.58-0.62 (2H, m)

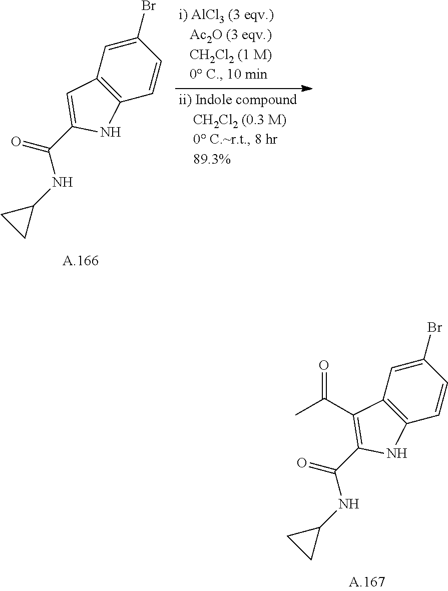

3-acetyl-5-bromo-N-cyclopropyl-1H-indole-2-carboxamide A.167 was prepared from compound A.166 by the procedure used to prepare compound A.164: Mass Spectrum (ESI) m/e=321.0 [M+1 (⁷⁹Br)] and 322.9 [M+1 (⁸¹Br)].

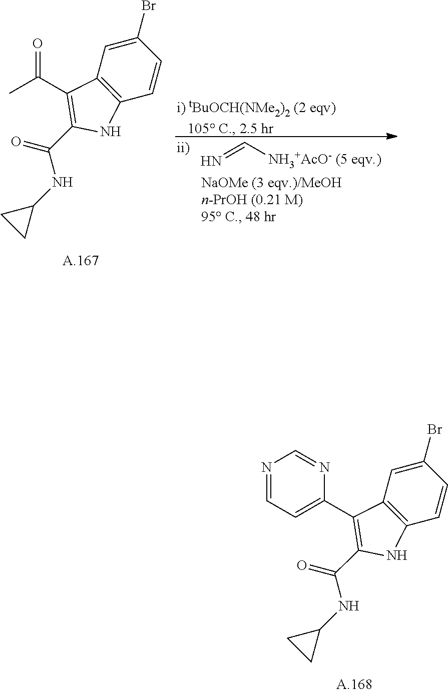

5-bromo-N-cyclopropyl-3-(pyrimidin-4-yl)-1H-indole-2-carboxamide A.168 was prepared from compound A.167 by the procedure used to prepare compound A.165: Mass Spectrum (ESI) m/e=357.0 [M+1 (⁷⁹Br)] and 359.0 [M+1 (⁸¹Br)].

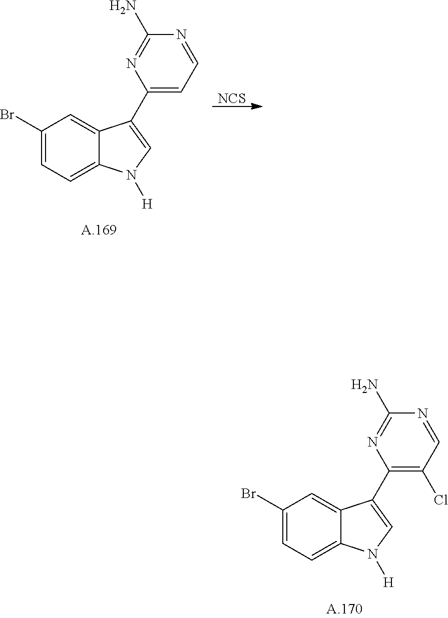

4-(5-bromo-1H-indol-3-yl)-5-chloropyrimidin-2-amine A.170: A mixture of N-chlorosuccinimide (500 mg, 3.74 mmol) and 4-(5-bromo-1H-indol-3-yl)pyrimidin-2-amine (A.169) (1.09 g, 3.74 mmol) [Fresneda, P. M., P. Molina, et al. (2000). Tetrahedron Lett. 41(24): 4777-4780] in CH₃CN (30 mL) was refluxed for 5 hrs. After cooling to room temperature, the mixture was triturated with ether. After filtration The product was purified by flash chromatography over silica gel, using 9.5:0.5 dichloromethane-methanol, to afford 4-(5-bromo-1H-indol-3-yl)-5-chloropyrimidin-2-amine (A.170)(810 mg, 67%): ¹H NMR (methanol-d₄) δ 8.78 (s, 1H), 8.47 (s, 1H), 8.21 (s, 1H), 7.41 (d, J=8.5 Hz, 1H), 7.35 (d, J=8.5 Hz, 1H), 2.04 (s, 2H); ms 325.1 (M+H⁺).

The following compounds A.171, A.172, and A.173 were prepared using the same or analogous synthetic techniques and substituting with appropriate reagents as example A.170.

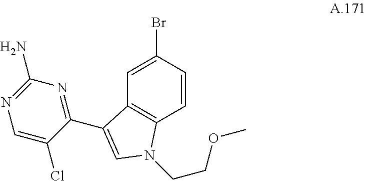

4-(5-bromo-1-(2-methoxyethyl)-1H-indol-3-yl)-5-chloropyrimidin-2-amine A.171 prepared from A.180: ms 381.0 (M+H⁺).

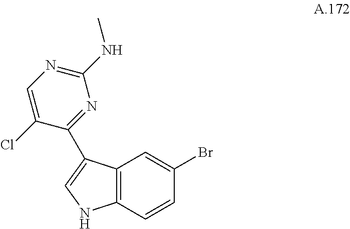

4-(5-bromo-1H-indol-3-yl)-5-chloro-N-methylpyrimidin-2-amine A.172: prepared from A.177. ¹H NMR (DMSO-d₆) δ 11.35 (s, 1H), 8.29 (br, 1H), 7.57-7.48 (m, 1H), 7.38-7.24 (m, 2H), 6.98-6.90 (m, 1H), 2.89 (br, 3H); ms 337.0/339.0 (M+H⁺).

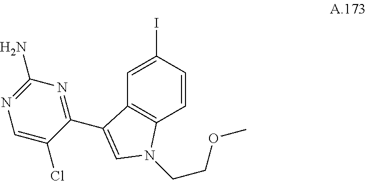

5-chloro-4-(5-iodo-1-(2-methoxyethyl)-1H-indol-3-yl)pyrimidin-2-amine A.173: prepared from A.181. ¹H NMR (DMSO-d₆) δ 8.91 (s, 1H), 8.44 (s, 1H), 8.20 (s, 1H), 7.52-7.45 (m, 2 H), 6.82 (s, 2H), 4.48-4.42 (m, 2H), 3.72-3.55 (m, 2H), 3.22 (s, 3H).

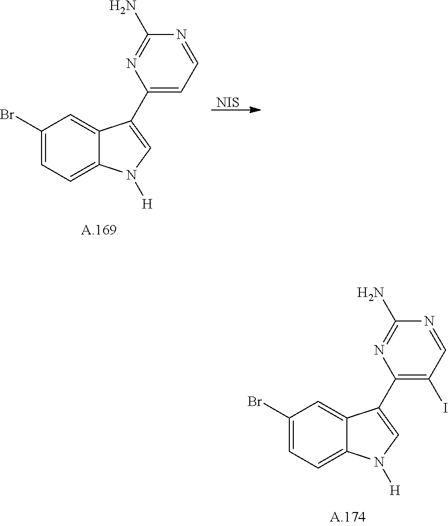

### 4-(5-bromo-1H-indol-3-yl)-5-iodopyrimidin-2-amine

A mixture of N-iodosuccinimide (1.17 g, 5.19 mmol) and 4-(5-bromo-1H-indol-3-yl)pyrimidin-2-amine (A.169) (500 mg, 1.73 mmol) in DMF (10 mL) was heated at 100° C. under N₂ for 1.5 hrs. After cooling to room temperature, the resultant mixture was poured onto ice. The precipitate was collected by filtration to afford 4-(5-bromo-1H-indol-3-yl)-5-iodopyrimidin-2-amine (A.174) (322.0 mg, 45%): ¹H NMR (DMSO-d₆) δ 11.86 (s, 1H), 8.56 (s, 1H), 8.44 (s, 1H), 8.37 (s, 1H), 7.43 (d, J=8.8 Hz, 1H), 7.29 (d, J=8.8 Hz, 1H), 6.78 (s, 2H); ms 415.0 (M+H⁺).

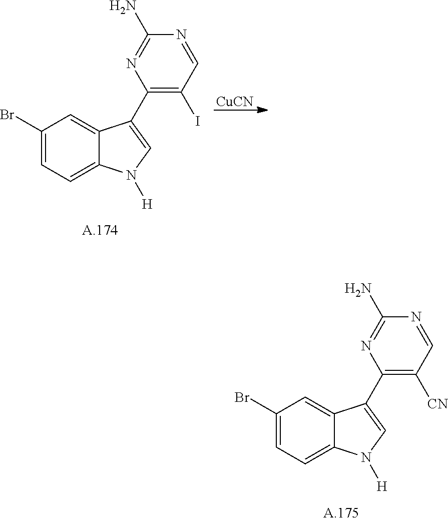

2-amino-4-(5-bromo-1H-indol-3-yl)pyrimidine-5-carbonitrile A.175: A mixture of 4-(5-bromo-1H-indol-3-yl)-5-iodopyrimidin-2-amine (A.174) (322.0 mg, 0.776 mmol) and CuCN (69.5 mg, 0.776 mmol) in DMF (10 mL) was heated at 100° C. under N₂ for 16 hrs. After cooling to room temperature, the resultant mixture was diluted with ether, washed with water and brine, dried and concentrated. Purification of the residue by flash chromatography over silica gel, using 5:4.5:0.5 ethyl acetate-hexane-methanol, gave 2-amino-4-(5-bromo-1H-indol-3-yl)pyrimidine-5-carbonitrile (A.175)(115.4 mg, 47%): ¹H NMR (DMSO-d₆) δ 12.12 (s, 1H), 8.78 (s, 1H), 8.60 (s, 1H), 8.51 (s, 1H), 7.84 (br, 1H), 7.59 (br, 1H), 7.50 (d, J=8.0 Hz, 1H), 7.38 (d, J=8.0 Hz, 1H); ms 314.0 (M+H⁺).

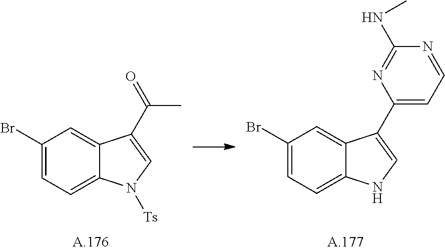

4-(5-bromo-1H-indol-3-yl)-N-methylpyrimidin-2-amine A.177: A mixture of 1-(5-bromo-1-tosyl-1H-indol-3-yl)ethanone (A.176) [Fresneda, P. M., P. Molina, et al. (2000). Tetrahedron Lett. 41(24): 4777-4780] (5.0 g, 12.8 mmol) and t-BuOCH(NMe₂)₂ (5.3 mL, 25.6 mmol) was heated at 105° C. for 3 hrs. At room temperature, n-BuOH (15 mL), 1-methylguanidine (HCl salt, 2.1 g, 19.2 mmol) and MeONa (5.33 M in methanol, 7.2 mL) were added sequentially to the mixture. The resultant mixture was heated at 95° C. for 16 hrs. After cooling to room temperature, the resultant mixture was concentrated, and the residue was diluted with water and neutralized with HCl (1M) to pH 7. The mixture was concentrated and dissolved in methanol. After filtration to remove insoluble solid, the filtrates were collected and concentrated. Purification of the residue by flash chromatography over silica gel, using 9.5:0.5 dichloromethane-methanol, gave 4-(5-bromo-1H-indol-3-yl)-N-methylpyrimidin-2-amine (A.177) (2.4 g, 62%): ¹H NMR (DMSO-d₆) δ 12.30 (s, 1H), 8.78 (s, 1H), 8.60 (s, 1H), 8.17 (d, J=6.0 Hz, 1H), 7.48 (br, 1H), 7.38 (d, J=8.5 Hz, 1H), 7.25 (d, J=6.0 Hz, 1H), 7.12 (d, J=8.5 Hz, 1H), 3.01 (br, 3H); ms 303.1 (M+H⁺).

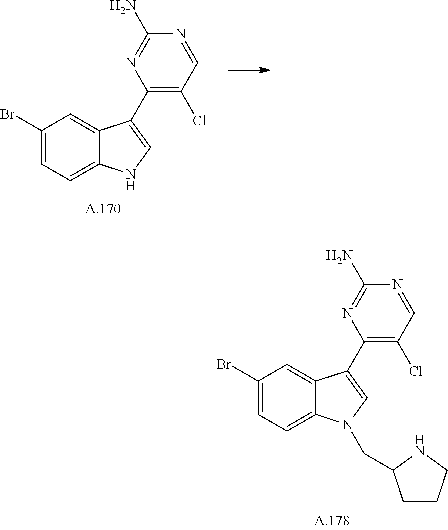

4-(5-bromo-1-(pyrrolidin-2-ylmethyl)-1H-indol-3-yl)-5-chloropyrimidin-2-amine (A.178): Maleic acid (2.86 g, 24.72 mmol) was added to pyrrolidin-2-ylmethanol (2.5 g, 24.72 mmol) (A.170) in ethyl acetate (10 mL). The resultant mixture was stirred at room temperature for 2 hrs. After concentration, NaHCO₃ (10.38 g, 123.6 mmol) in water (16 mL) was added to the residue. (BOC)₂O (6.47 g, 29.66 mmol) was then added. The mixture was stirred at room temperature for 16 hrs. After removing solid by filtration, the aqueous solution was extracted with ethyl acetate. The organics were dried and concentrated, gave crude N-BOC protected pyrrolidin-2-ylmethanol. To the crude N-BOC protected pyrrolidin-2-ylmethanol (2.5 g, 12.43 mmol) and triethylamine (4.16 ml, 29.82 mmol) in ethyl acetate at 0° C., MsCl (1.16 mL, 14.91 mmol) was added and the mixture was stirred at 0° C. for 2 hrs. The reaction was quenched with water and extracted with ethyl acetate. The organics were washed with 2N HCl, water, Sat. aq. NaHCO₃, and brine, dried and concentrated. A portion of the residue (257.6 mg, 0.924 mmol) was added to a mixture of Cs₂CO₃ (301.1 mg, 0.924 mmol) and 4-(5-bromo-1H-indol-3-yl)-5-chloropyrimidin-2-amine A.170 (100 mg, 0.308 mmol) in DMF (1 mL) and DMSO (1 mL). The mixture was heated at 110° C. for 16 hrs. After cooling to room temperature, the mixture was diluted with ether, washed with water and brine, dried and concentrated. A portion of the residue (68.5 mg, 0.135 mmol) was dissolved in dichloromethane (8 mL) and TFA (2 mL) then was added. The mixture was stirred at room temperature for 1 hr, concentrated and diluted with methanol-dichloromethane (1:9). The solution was washed with Sat. aq. NaHCO₃, and brine, dried and concentrated to afford 4-(5-bromo-1-(pyrrolidin-2-ylmethyl)-1H-indol-3-yl)-5-chloropyrimidin-2-amine (A.178) (32.0 mg, 58%): ¹H NMR (methanol-d₄) δ 8.78 (s, 1H), 8.43 (s, 1H), 8.15 (s, 1H), 7.42 (d, J=8.0 Hz, 1 H), 7.38 (d, J=8.0 Hz, 1H), 4.29-4.15 (m, 2H), 3.55-3.45 (m, 1H), 3.00-2.95 (m, 1H), 2.90-2.85 (m, 1H)), 1.92-1.80 (m, 2H), 1.80-1.72 (m, 1H), 1.55-1.46 (m, 1H); ms 406.0 (M+H⁺).

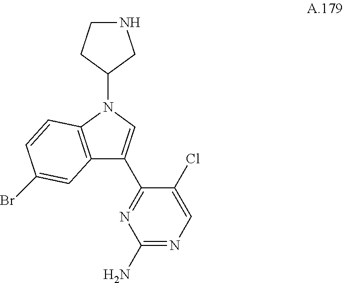

4-(5-bromo-1-(pyrrolidin-3-yl)-1H-indol-3-yl)-5-chloropyrimidin-2-amine A.179: This compound was prepared by using the same or analogous synthetic techniques and substituting with appropriate reagents as for compound A.178. ¹H NMR (DMSO-d₆) δ 8.72 (s, 1H), 8.56 (s, 1H), 8.26 (s, 1H), 7.66 (d, J=8.6 Hz, 1H), 7.44 (d, J=8.6 Hz, 1H), 6.87 (br, 2 H), 5.39 (br, 1H), 3.72-3.63 (m, 1H), 3.40-3.15 (m, 4H), 2.55-2.45 (m, 1H), 2.35-2.25 (m, 1H).

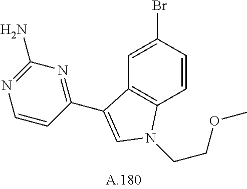

4-(5-bromo-1-(2-methoxyethyl)-1H-indol-3-yl)pyrimidin-2-amine A.180: The title compound was prepared from compound A.169 by the method used for example 124.: ¹H NMR (DMSO-d₆) δ 8.76 (s, 1H), 8.26 (s, 1H), 8.13 (d, J=5.0 Hz, 1H), 7.57 (d, J=8.0 Hz, 1 H), 7.35 (d, J=8.0 Hz, 1H), 6.96 (d, J=5.0 Hz, 1H), 6.52 (s, 2H), 4.41 (t, J=5.0 Hz, 2H), 3.71 (t, J=5.0 Hz, 2H), 3.24 (s, 3H).

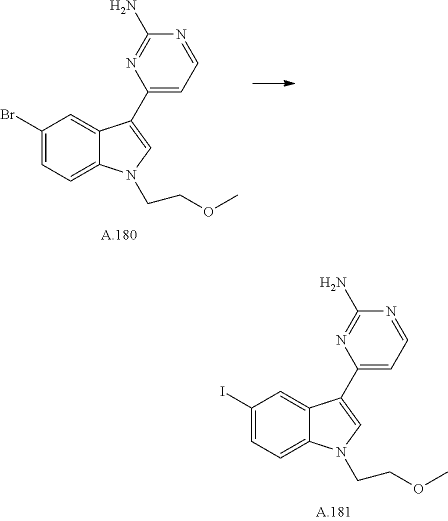

4-(5-iodo-1-(2-methoxyethyl)-1H-indol-3-yl)pyrimidin-2-amine A.181: To a mixture of 4-(5-bromo-1-(2-methoxyethyl)-1H-indol-3-yl)pyrimidin-2-amine (A.180) (445 mg, 1.28 mmol), NaI (383.7 mg, 2.56 mmol), and copper(I) iodide (42.0 mg, 0.22 mmol) in dioxane (20 mL) was added N¹,N²-dimethylethane-1,2-diamine (0.1 mL) under N₂ and heated at 110° C. under N₂ for 24 hrs. After cooled to room temperature, the resultant mixture was diluted with ether, washed with water and brine, dried and concentrated. Purification of the residue by flash chromatography over silica gel, using 4:5:1 ethyl acetate-hexane-methanol, gave 4-(5-iodo-1-(2-methoxyethyl)-1H-indol-3-yl)pyrimidin-2-amine (A.181) (358.9 mg, 70%): ¹H NMR (DMSO-d₆) δ 8.90 (s, 1H), 8.20 (s, 1H), 8.12 (d, J=8.4 Hz, 1H), 7.49 (d, J=8.4 Hz, 1H), 7.44 (d, J=8.4 Hz, 1H), 6.93 (d, J=4.8 Hz, 1H), 6.49 (s, 2H), 4.39 (t, J=4.8 Hz, 2H), 3.70 (t, J=4.8 Hz, 2H), 3.16 (s, 3H); ms 395.0 (M+H⁺).

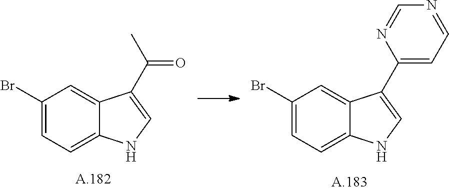

5-bromo-3-(pyrimidin-4-yl)-1H-indole A.183: To the mixture of 1-(5-bromo-1H-indol-3-yl)ethanone A.182 [Fresneda, P. M., P. Molina, et al. (2001). Tetrahedron 57(12): 2355-2364.] (500 mg, 2.1 mmol) and N,N′,N″-Methylidynetrisformamide (610 mg, 4.2 mmol) in NMP (5 ml) TsOH (199.7 mg, 1.05 mmol) was added. The mixture was heated at 185° C. for 2 days. After cooled to room temperature, the resultant mixture was diluted with ethyl acetate, washed with water and brine, dried and concentrated. Purification of the residue by flash chromatography over silica gel, using 9.5:0.5 dichloromethane-methanol, gave 5-bromo-3-(pyrimidin-4-yl)-1H-indole (A.183) (118.6 mg, 74%): ¹H NMR (DMSO-d₆) δ 9.24 (s, 1H), 8.72 (d, J=1.8 Hz, 1H), 8.63 (d, J=5.6 Hz, 1H), 8.48 (s, 1H), 7.90 (dd, J=5.6, 1.8 Hz, 1H), 7.46 (d, J=8.6 Hz, 1H), 7.34 (dd, J=8.6, 1.5 Hz, 1H); ms 274.0 (M+H⁺).

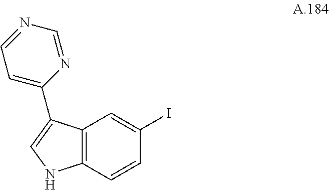

5-iodo-3-(pyrimidin-4-yl)-1H-indole A.184: This compound was prepared from A.183 by the procedure used for A.181: ¹H NMR (DMSO-d₆) δ 12.03 (s, 1H), 9.13 (s, 1H), 8.92 (s, 1 H), 8.63 (d, J=5.6 Hz, 1H), 8.42 (s, 1H), 7.89 (d, J=5.6 Hz, 1H), 7.49 (d, J=8.8 Hz, 1H), 7.24 (d, J=8.8 Hz, 1H); ms 322.0 (M+H⁺).

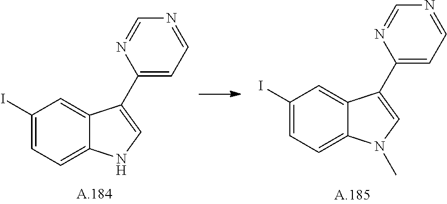

5-iodo-1-methyl-3-(pyrimidin-4-yl)-1H-indole A.185: A mixture of 5-iodo-3-(pyrimidin-4-yl)-1H-indole (A.184) (180 mg, 0.561 mmol), Dabco (6.3 mg, 0.0561 mmol), DMF (0.5 mL) and dimethyl carbonate (5 mL) was heated at 95° C. for 16 hrs. After cooling to room temperature, the resultant mixture was diluted with ethyl acetate, washed with water, aq.NH₄Cl and brine, dried and concentrated. Purification of the residue by flash chromatography over silica gel, using 9.5:0.5 dichloromethane-methanol, gave 5-iodo-1-methyl-3-(pyrimidin-4-yl)-1H-indole (A.185) (130 mg, 69%): ¹H NMR (DMSO-d₆) δ 9.13 (s, 1H), 8.92 (d, J=1.8 Hz, 1 H), 8.63 (d, J=5.6 Hz, 1H), 8.45 (s, 1H), 7.81 (d, J=5.6 Hz, 1H), 7.58 (d, J=8.6 Hz, 1H), 7.44 (d, J=8.6 Hz, 1H), 3.83 (s, 1H); ms 336.0 (M+H⁺).

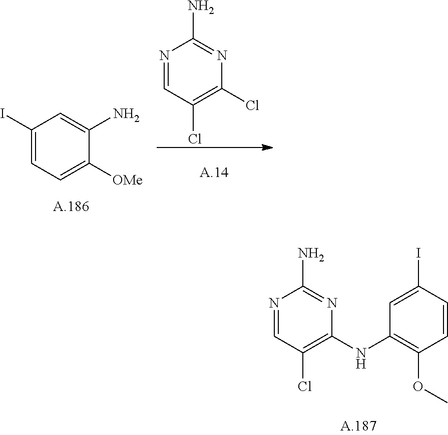

5-Chloro-N⁴-(5-iodo-2-methoxyphenyl)pyrimidine-2,4-diamine (A.187). To a flask containing 5-iodo-2-methoxybenzenamine (A.186) (472 mg, 1.9 mmol) in 1,4-dioxane (8.00 mL) was added 4,5-dichloropyrimidin-2-amine A.14 (622 mg, 3.8 mmol) and 1 N HCl (7.00 mL). The reaction was heated to 80° C. After 3 hours, the mixture was cooled to room temperature then carefully neutralized with 1N NaOH. After extracting with ethyl acetate, the solvent was removed under reduced pressure. Methanol was then added to the residue and heated to 50° C. After 15 minutes, the solvent was removed to a volume of ˜1.0 mL and cooled to room temperature. The solid was filtered and dried to yield 5-chloro-N⁴-(5-iodo-2-methoxyphenyl)pyrimidine-2,4-diamine (A.187) (341 mg, 47.7%). ¹H NMR (500 MHz, DMSO-d₆) δ ppm 8.53 (1H, d, J=2.2 Hz), 7.97 (1H, s), 7.85 (1H, s), 7.40 (1H, dd, J=8.6, 2.2 Hz), 6.90 (1H, d, J=8.6 Hz), 6.58 (2H, s), 3.86 (3H, s). MS ESI (pos.) m/e: 376.9 (M+H).

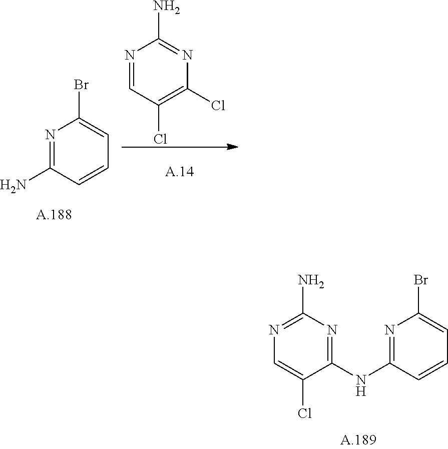

N⁴-(6-Bromopyridin-2-yl)-5-chloropyrimidine-2,4-diamine (A.189). To a dry flask containing 6-bromopyridin-2-amine A.188 (1.25 g, 7.23 mmol) and dry DMF (20.0 mL) at 0° C. was added potassium tert-butoxide (973.9 mg, 8.68 mmol) in portions. After 1 hour, 4,5-dichloropyrimidin-2-amine A.14 (1.18 g, 7.22 mmol) was added to the mixture then heated to 50° C. Upon completion, the reaction was cooled to room temperature, then quenched with water. After extracting three times with ethyl acetate, the organic solvent was removed under reduced pressure. Methanol was added to the residue and heated to 45° C. After 30 minutes, the solid was filtered hot then rinsed three times with methanol to yield N⁴-(6-bromopyridin-2-yl)-5-chloropyrimidine-2,4-diamine (A.189). (1.012 g, 47%) ¹H NMR (500 MHz, DMSO-d₆) δ ppm 8.56 (1H, s), 8.39 (1H, m), 8.06 (1H, m), 7.72 (1H, m), 7.30 (1H, m), 6.74 (2H, s).

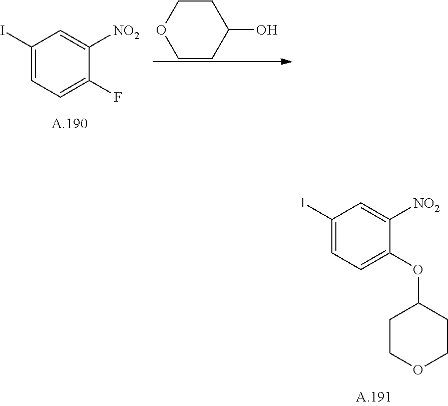

4-(4-Iodo-2-nitrophenoxy)-tetrahydro-2H-pyran (A.191). A dry flask containing tetrahydro-2H-pyran-4-ol (0.8 mL, 8.39 mmol) in dry DMF (13.00 mL) was cooled in an ice bath, then sodium hydride (60% wt. dispersion in oil) (336.2 mg, 8.41 mmol) was carefully added in portions. After 15 minutes, 1-fluoro-4-iodo-2-nitrobenzene A.190 [Prakash, G. K. S., T. Mathew, et al. (2004). J. Amer. Chem. Soc. 126(48): 15770-15776.] was added, and the mixture was stirred at room temperature. After 21 hours, the reaction was carefully quenched with cold water then extracted three times with ethyl acetate. After removing the organic solvent under reduced pressure, the residue was purified by silica gel flash chromatography (0-50% ethyl acetate in hexanes) to afford 4-(4-iodo-2-nitrophenoxy)-tetrahydro-2H-pyran (A.191) (2.02 g, 88%). ¹H NMR (500 MHz, DMSO-d₆) δ ppm 8.16 (1H, d, J=2.2 Hz), 7.90 (1 H, dd, J=8.8, 2.2 Hz), 7.29 (1H, t, J=9.4 Hz), 4.87 (1H, m), 3.82 (2H, m), 3.49 (2H, ddd, J=11.4, 7.9, 3.2 Hz), 1.99 (2H, m), 1.64 (2H, m).

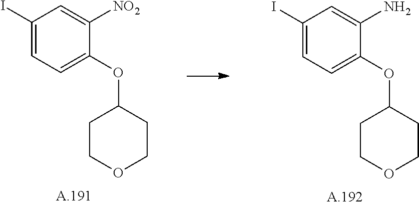

5-Iodo-2-(tetrahydro-2H-pyran-4-yloxy)benzenamine (A.192). To a flask containing 4-(4-iodo-2-nitrophenoxy)-tetrahydro-2H-pyran (A.191) (2.02 g, 5.78 mmol) and acetic acid (35.00 mL) was added iron powder (1.29 g, 23.1 mmol). The reaction was carefully heated to 100° C. After 1.25 hours, the reaction was cooled to room temperature then filtered through celite. After rinsing the filter cake three times with ethyl acetate, the filtrate was washed once with brine then concentrated. The residue was purified by silica gel flash chromatography (0-100% ethyl acetate in hexanes) to afford 5-iodo-2-(tetrahydro-2H-pyran-4-yloxy)benzenamine (A.192) (1.40 g, 76%). ¹H NMR (500 MHz, DMSO-d₆) δ ppm 6.96 (1H, d, J=2.0 Hz), 6.77 (1 H, m), 6.65 (1H, d, J=8.3 Hz), 4.93 (2H, s), 4.46 (1H, m), 3.87 (2H, m), 3.46 (2H, m), 1.91 (2H, m), 1.63 (2H, m). MS ESI (pos.) m/e: 320.0 (M+H).

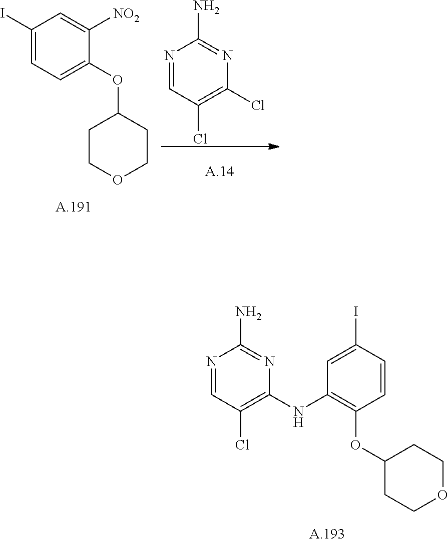

### 5-Chloro-N⁴-(5-iodo-2-(tetrahydro-2H-pyran-4-yloxy)phenyl)pyrimidine-2,4-diamine (A.193)

To a flask containing 5-iodo-2-(tetrahydro-2H-pyran-4-yloxy)benzenamine (A.192) (1.40 g, 4.38 mmol) in 1,4-dioxane (11.00 mL) was added 4,5-dichloropyrimidin-2-amine A.14 (1.44 g, 8.75 mmol) and 1 N HCl (9.00 mL). The reaction was heated to 80° C. After 21 hours, the mixture was cooled to room temperature then carefully neutralized with 1N NaOH. After extracting with ethyl acetate, the solvent was removed under reduced pressure. Methanol was then added to the residue and heated to 50° C. After 15 minutes, the solvent was removed to a volume of ˜1.0 mL and cooled to room temperature. The solid was filtered and dried to yield 5-chloro-N⁴-(5-iodo-2-(tetrahydro-2H-pyran-4-yloxy)phenyl)pyrimidine-2,4-diamine (A.193) (698 mg, 36%). ¹H NMR (500 MHz, DMSO-d₆) δ ppm 8.66 (1H, d, J=2.0 Hz), 8.00 (2H, m), 7.35 (1H, dd, J=8.6, 2.0 Hz), 7.02 (1H, m), 6.64 (2H, s), 4.69 (1H, dd, J=7.3, 3.7 Hz), 3.85 (2H, m), 3.50 (2H, ddd, J=11.2, 8.1, 2.9 Hz), 1.97 (2H, d, J=12.5 Hz), 1.66 (2H, m). MS ESI (pos.) m/e: 447.0 (M+H).

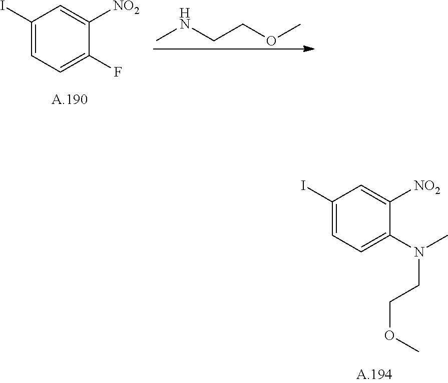

4-Iodo-N-(2-methoxyethyl)-N-methyl-2-nitrobenzenamine (A.194). A dry flask containing 2-methoxy-N-methylethanamine (1.2 mL, 11.2 mmol) in dry DMF (5.00 mL) was cooled in an ice bath, then sodium hydride (60% wt. dispersion in oil) (448.6 mg, 11.2 mmol) was carefully added in portions. After 15 minutes, 1-fluoro-4-iodo-2-nitrobenzene A.190 (2.51 g, 9.4 mmol) was added, and the mixture was stirred at room temperature. After 19 hours, the reaction was carefully quenched with cold water then extracted three times with ethyl acetate. After removing the organic solvent under reduced pressure, the residue was purified by silica gel flash chromatography (0-30% ethyl acetate in hexanes) to afford 4-iodo-N-(2-methoxyethyl)-N-methyl-2-nitrobenzenamine (A.194) (2.24 g, 71%). ¹H NMR (500 MHz, MeOH) δ ppm 7.95 (1H, d, J=2.0 Hz), 7.69 (1H, dd, J=8.8, 2.2 Hz), 7.06 (1H, d, J=8.8 Hz), 3.57 (2H, t, J=5.5 Hz), 3.37 (5H, m), 2.86 (3H, s). MS ESI (pos.) m/e: 337.0 (M+H).

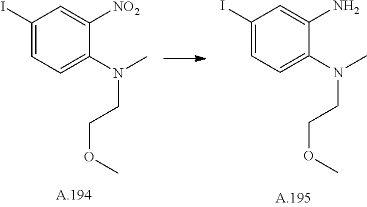

4-Iodo-N¹-(2-methoxyethyl)-N¹-methylbenzene-1,2-diamine (A.195). To a flask containing 4-iodo-N-(2-methoxyethyl)-N-methyl-2-nitrobenzenamine (A.194) (2.24 g, 6.65 mmol) and acetic acid (30.00 mL) was added iron powder (1.49 g, 26.7 mmol). The reaction was carefully heated to 100° C. After 1 hour, the reaction was cooled to room temperature then filtered through celite. After rinsing the filter cake three times with ethyl acetate, the filtrate was washed once with brine then concentrated. The residue was purified by silica gel flash chromatography (0-100% ethyl acetate in hexanes) to afford 4-iodo-N¹-(2-methoxyethyl)-N¹-methylbenzene-1,2-diamine (A.195) (1.20 g, 59%). ¹H NMR (500 MHz, DMSO-d₆) δ ppm 6.96 (1H, d, J=2.0 Hz), 6.79 (1H, dd, J=8.3, 2.0 Hz), 6.70 (1H, d, J=8.1 Hz), 5.07 (2H, s), 3.43 (2H, t, J=5.5 Hz), 3.27 (3H, s), 2.86 (2H, t, J=5.5 Hz), 2.58 (3H, s). MS ESI (pos.) m/e: 307.0 (M+H).

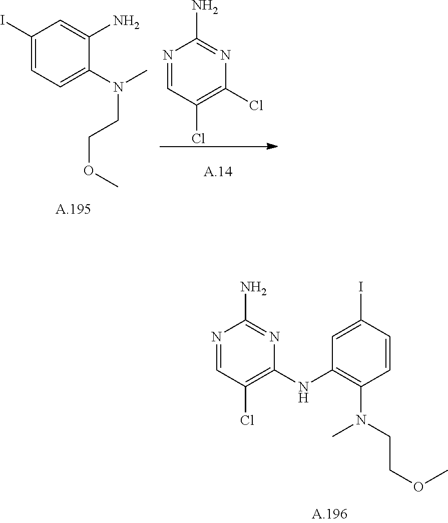

5-Chloro-N⁴-(5-iodo-2-((2-methoxyethyl)(methyl)amino)phenyl)pyrimidine-2,4-diamine (A.196). To a flask containing 4-iodo-N¹-(2-methoxyethyl)-N¹-methylbenzene-1,2-diamine (A.195) (1.20 g, 3.92 mmol) in 1,4-dioxane (11.00 mL) was added 4,5-dichloropyrimidin-2-amine A.14 (1.29 g, 7.84 mmol) and 1 N HCl (9.00 mL). The reaction was heated to 80° C. After 19.5 hours, the mixture was cooled to room temperature then carefully neutralized with 1N NaOH. After extracting with ethyl acetate, the solvent was removed under reduced pressure. The residue was purified by silica gel flash chromatography (0-40% ethyl acetate in dichloromethane) to afford 5-chloro-N⁴-(5-iodo-2-((2-methoxyethyl)(methyl)amino)phenyl)-pyrimidine-2,4-diamine (A.196) (753 mg, 44%). ¹H NMR (500 MHz, DMSO-d₆) δ ppm 8.77 (2H, m), 7.99 (1H, m), 7.37 (1H, dd, J=8.2, 2.1 Hz), 7.11 (1H, d, J=8.3 Hz), 6.63 (2H, s), 3.36 (3H, m), 3.17 (3H, m), 2.98 (2H, t, J=5.6 Hz), 2.63 (3H, s). MS ESI (pos.) m/e: 434.0 (M+H).

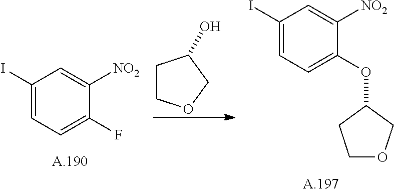

(S)-3-(4-Iodo-2-nitrophenoxy)-tetrahydrofuran (A.197). A dry flask containing 2(S)-tetrahydrofuran-3-ol (0.55 mL, 6.89 mmol) in dry DMF (10.00 mL) was cooled in an ice bath, then sodium hydride (60% wt. dispersion in oil) (275.5 mg, 6.89 mmol) was carefully added in portions. After 15 minutes, 1-fluoro-4-iodo-2-nitrobenzene A.190 (1.47 g, 5.52 mmol) was added, and the mixture was stirred at room temperature. After 19.5 hours, the reaction was carefully quenched with cold water then extracted three times with ethyl acetate. After removing the organic solvent under reduced pressure, the residue was purified by silica gel flash chromatography (0-30% ethyl acetate in hexanes) to afford (S)-3-(4-iodo-2-nitrophenoxy)-tetrahydrofuran (A.197) (1.58 g, 85%). ¹H NMR (500 MHz, DMSO-d₆) δ ppm 8.18 (1H, m), 7.95 (1H, m), 7.20 (1H, d, J=9.0 Hz), 5.23 (1H, d, J=4.4 Hz), 3.91 (1H, m), 3.81 (3H, m), 2.26 (1H, m), 1.99 (1H, m).

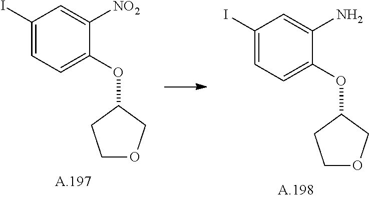

(S)-5-Iodo-2-(tetrahydrofuran-3-yloxy)benzenamine (A.198). To a flask containing (S)-3-(4-iodo-2-nitrophenoxy)-tetrahydrofuran (A.197) (1.58 g, 4.71 mmol) and acetic acid (35.00 mL) was added iron powder (1.05 g, 18.8 mmol). The reaction was carefully heated to 100° C. After 1.75 hours, the reaction was cooled to room temperature then filtered through celite. After rinsing the filter cake three times with ethyl acetate, the filtrate was washed once with brine then concentrated. The residue was purified by silica gel flash chromatography (0-100% ethyl acetate in hexanes) to afford (S)-5-iodo-2-(tetrahydrofuran-3-yloxy)benzenamine (A.198) (418.6 mg, 29%). ¹H NMR (500 MHz, DMSO-d₆) δ ppm 6.94 (1H, d, J=2.0 Hz), 6.77 (1H, dd, J=8.3, 2.2 Hz), 6.57 (1H, m), 4.93 (1H, s), 4.91 (2H, ddd, J=6.4, 4.3, 2.3 Hz), 3.88 (2H, m), 3.80 (2H, m), 2.19 (1H, m), 2.01 (1H, m). MS ESI (pos.) m/e: 306.0 (M+H).

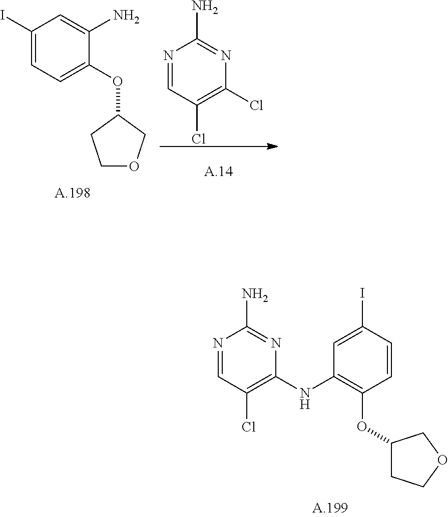

(S)-5-Chloro-N⁴-(5-iodo-2-(tetrahydrofuran-3-yloxy)phenyl)pyrimidine-2,4-diamine (A.199). To a flask containing (S)-5-iodo-2-(tetrahydrofuran-3-yloxy)benzenamine (A.198) (418.6 mg, 1.37 mmol) in 1,4-dioxane (5.00 mL) was added 4,5-dichloropyrimidin-2-amine A.14 (450.6 mg, 2.75 mmol) and 1 N HCl (5.00 mL). The reaction was heated to 80° C. After 8 hours, the mixture was cooled to room temperature then carefully neutralized with 1N NaOH. After extracting with ethyl acetate, the solvent was removed under reduced pressure. Methanol was then added to the residue and heated to 50° C. After 15 minutes, the solvent was removed to a volume of ˜1.0 mL and cooled to room temperature. The solid was filtered and dried to yield (S)-5-chloro-N⁴-(5-iodo-2-(tetrahydrofuran-3-yloxy)phenyl)pyrimidine-2,4-diamine (A.199) (451.1 mg, 76%). ¹H NMR (500 MHz, DMSO-d₆) δ ppm 8.61 (1H, d, J=1.2 Hz), 7.98 (1H, s), 7.89 (1H, s), 7.37 (1H, dd, J=8.6, 2.0 Hz), 6.91 (1H, d, J=8.6 Hz), 6.61 (2 H, s), 5.14 (1H, m), 3.89 (1H, dd, J=10.5, 4.4 Hz), 3.83 (1H, q, J=7.7 Hz), 3.79 (2H, m), 2.22 (1H, td, J=14.1, 8.3 Hz), 2.03 (1H, m). MS ESI (pos.) m/e: 432.9 (M+H).

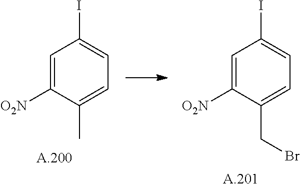

1-(Bromomethyl)-4-iodo-2-nitrobenzene (A.201). To a flask containing 4-iodo-1-methyl-2-nitrobenzene A.200 (2.52 g, 9.57 mmol) in dry CCl₄ (35.0 mL) was added N-bromosuccimide (1.88 g, 10.5 mmol) and benzoyl peroxide (47.0 mg, 0.19 mmol). The mixture was heated to reflux. After 20 hours, the reaction was cooled to room temperature then diluted with water. After extracting three times with dichloromethane, the organic solvent was removed under reduced pressure. The residue was purified by silica gel flash chromatography (6:1 hexane in dichloromethane) to afford 1-(bromomethyl)-4-iodo-2-nitrobenzene (A.201) (1.37 g, 42%).

¹H NMR (500 MHz, DMSO-d₆) δ ppm 8.38 (1H, m), 8.11 (1H, dd, J=8.1, 1.7 Hz), 7.55 (1H, m), 4.86 (2H, s).

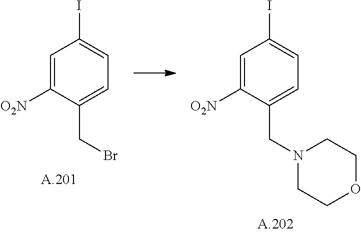

4-(4-Iodo-2-nitrobenzyl)morpholine (A.202). To a flask containing 1-(bromomethyl)-4-iodo-2-nitrobenzene (A.201) (1.30 g, 3.79 mmol) was added THF (20.0 mL) and morpholine (0.63 mL, 7.22 mmol). The mixture was stirred at room temperature for 37 hours then diluted with water. After extracting three times with dichloromethane, the organic solvent was removed under reduced pressure to yield 4-(4-iodo-2-nitrobenzyl)morpholine (A.202).

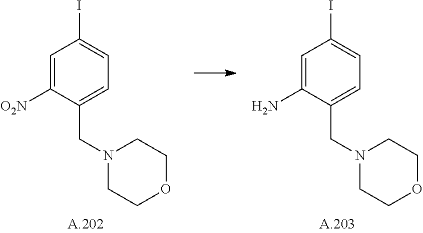

5-Iodo-2-(morpholinomethyl)benzenamine (A.203). To a flask containing 4-(4-iodo-2-nitrobenzyl)morpholine (A.202) (1.01 g, 2.90 mmol) and acetic acid (20.00 mL) was added iron powder (648.1 mg, 11.6 mmol). The reaction was carefully heated to 100° C. After 2 hours, the reaction was cooled to room temperature then filtered through celite. After rinsing the filter cake three times with ethyl acetate, the filtrate was washed once with brine then concentrated. The residue was purified by silica gel flash chromatography (0-100% ethyl acetate in hexanes) to afford 5-iodo-2-(morpholinomethyl)benzenamine (A.203) (817.9 mg, 68% for two steps.) ¹H NMR (500 MHz, DMSO-d₆) δ ppm 7.01 (1H, m), 6.82 (1H, m), 6.72 (1H, d, J=7.8 Hz), 5.44 (2H, s), 3.58 (4H, m), 2.29 (4H, m). MS ESI (pos.) m/e: 319.0 (M+H).

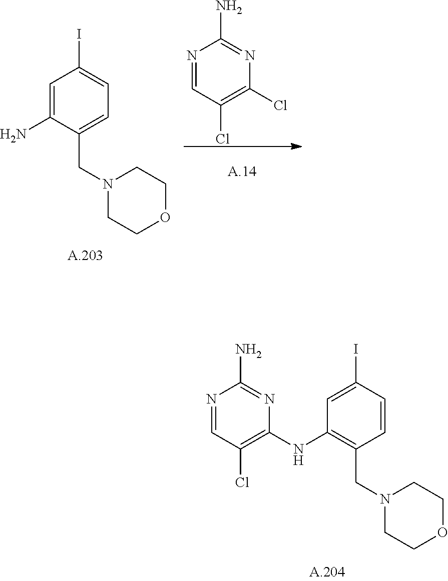

5-Chloro-N⁴-(5-iodo-2-(morpholinomethyl)phenyl)pyrimidine-2,4-diamine (A.204). To a flask containing 5-iodo-2-(morpholinomethyl)benzenamine (A.203) (817.9 mg, 2.57 mmol) in 1,4-dioxane (5.00 mL) was added 4,5-dichloropyrimidin-2-amine A.14 (842.9 mg, 5.14 mmol) and 1 N HCl (5.00 mL). The reaction was heated to 80° C. After 8 hours, the mixture was cooled to room temperature then carefully neutralized with 1N NaOH. After extracting with dichloromethane, the solvent was removed under reduced pressure. Methanol was then added to the residue and heated to 50° C. After 15 minutes, the solvent was removed to a volume of ˜1.0 mL and cooled to room temperature. The solid was filtered and dried to yield 5-chloro-N⁴-(5-iodo-2-(morpholinomethyl)phenyl)-pyrimidine-2,4-diamine (A.204) (502.1 mg, 44%). ¹H NMR (500 MHz, DMSO-d₆) δ ppm 9.93 (1H, s), 8.60 (1H, s), 7.98 (1H, s), 7.36 (1H, d, J=7.8 Hz), 7.01 (1H, d, J=7.8 Hz), 6.53 (2H, s), 3.60 (4H, m), 3.53 (2H, s), 2.38 (4 H, m). MS ESI (pos.) m/e: 445.9 (M+H).

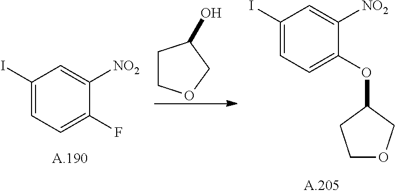

(R)-3-(4-Iodo-2-nitrophenoxy)-tetrahydrofuran (A.205). A dry flask containing 2(R)-tetrahydrofuran-3-ol (0.38 mL, 4.73 mmol) in dry DMF (10.00 mL) was cooled in an ice bath, then sodium hydride (60% wt. dispersion in oil) (189.6 mg, 4.74 mmol) was carefully added in portions. After 15 minutes, 1-fluoro-4-iodo-2-nitrobenzene A.190 (1.01 g, 3.78 mmol) was added, and the mixture was stirred at room temperature. After 21 hours, the reaction was carefully quenched with cold water then extracted three times with ethyl acetate. After removing the organic solvent under reduced pressure, the residue was purified by silica gel flash chromatography (0-30% ethyl acetate in hexanes) to afford (R)-3-(4-iodo-2-nitrophenoxy)-tetrahydrofuran (A.205) (1.42 g, 89%). ¹H NMR (500 MHz, DMSO-d₆) δ ppm 8.18 (1H, m), 7.94 (1H, m), 7.18 (1H, t, J=9.4 Hz), 5.24 (1H, m), 3.91 (1H, m), 3.81 (3H, m), 2.27 (1H, m), 1.99 (1H, m).

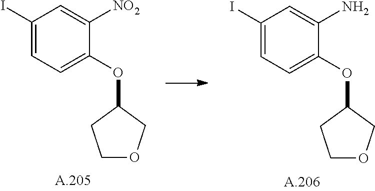

(R)-5-Iodo-2-(tetrahydrofuran-3-yloxy)benzenamine (A.206). To a flask containing (R)-3-(4-iodo-2-nitrophenoxy)-tetrahydrofuran (A.205) (1.42 g, 4.22 mmol) and acetic acid (15.00 mL) was added iron powder (941.8 mg, 16.9 mmol). The reaction was carefully heated to 100° C. After 1.5 hours, the reaction was cooled to room temperature then filtered through celite. After rinsing the filter cake three times with ethanol, the organic solvent was removed under reduced pressure to afford (R)-5-iodo-2-(tetrahydrofuran-3-yloxy)benzenamine (A.206) (501.9 mg, 39%). ¹H NMR (500 MHz, DMSO-d₆) δ ppm 6.94 (1H, d, J=2.2 Hz), 6.76 (1H, dd, J=8.3, 2.2 Hz), 6.55 (1H, d, J=8.3 Hz), 4.93 (3H, m), 3.88 (2H, m), 3.80 (2H, m), 2.19 (1 H, m), 2.01 (1H, m). MS ESI (pos.) m/e: 306.0 (M+H).

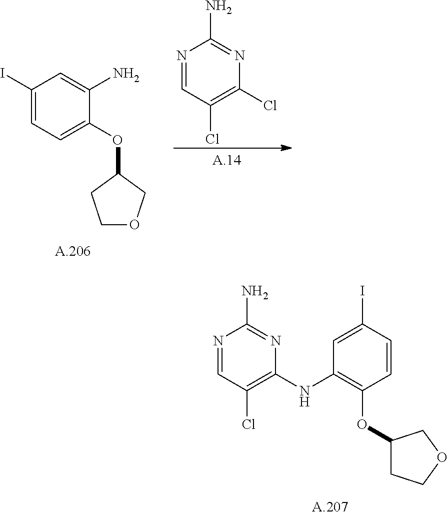

(R)-5-Chloro-N⁴-(5-iodo-2-(tetrahydrofuran-3-yloxy)phenyl)pyrimidine-2,4-diamine (A.207). To a flask containing (R)-5-iodo-2-(tetrahydrofuran-3-yloxy)benzenamine (A.206) (501.9 mg, 1.64 mmol) in 1,4-dioxane (5.00 mL) was added 4,5-dichloropyrimidin-2-amine (A.14)(539.8 mg, 3.29 mmol) and 1 N HCl (5.00 mL). The reaction was heated to 80° C. After 19 hours, the mixture was cooled to room temperature then carefully neutralized with 1N NaOH. After extracting with ethyl acetate, the solvent was removed under reduced pressure. Methanol was then added to the residue and heated to 50° C. After 15 minutes, the solvent was removed to a volume of ˜1.0 mL and cooled to room temperature. The solid was filtered and dried to yield (R)-5-chloro-N⁴-(5-iodo-2-(tetrahydrofuran-3-yloxy)phenyl)pyrimidine-2,4-diamine (A.207) (492.3 mg, 69%). ¹H NMR (500 MHz, DMSO-d₆) δ ppm 8.61 (1H, s), 7.98 (1H, s), 7.89 (1H, s), 7.38 (1H, d, J=2.0 Hz), 6.91 (1H, d, J=8.6 Hz), 6.62 (2H, s), 5.12 (1 H, s), 3.89 (1H, s), 3.84 (3H, m), (1H, m), 2.01 (1H, m). MS ESI (pos.) m/e: 433.0 (M+H).

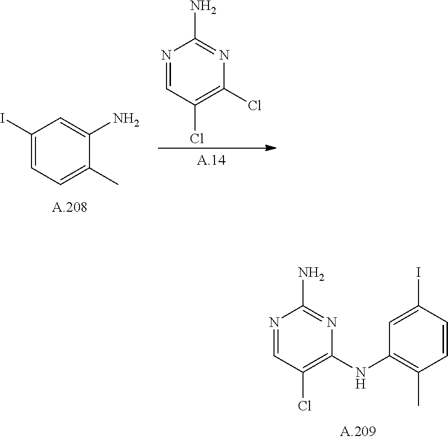

5-Chloro-N⁴-(5-iodo-2-methylphenyl)pyrimidine-2,4-diamine (A.209). To a flask containing 5-iodo-2-methylbenzenamine (A.208)(500.6 mg, 2.15 mmol) in 1,4-dioxane (5.00 mL) was added 4,5-dichloropyrimidin-2-amine (A.14)(704.7 mg, 4.30 mmol) and 1 N HCl (5.00 mL). The reaction was heated to 80° C. After 8 hours, the mixture was cooled to room temperature then carefully neutralized with 1N NaOH. After extracting with ethyl acetate, the solvent was removed under reduced pressure. Isopropanol was then added to the residue and heated to 50° C. After 15 minutes, the solvent was removed to a volume of ˜1.0 mL and cooled to room temperature. The solid was filtered and dried to yield 5-chloro-N⁴-(5-iodo-2-methylphenyl)pyrimidine-2,4-diamine (A.209). (352.3 mg, 45%). ¹H NMR (500 MHz, DMSO-d₆) δ ppm 8.34 (1H, s), 7.87 (1H, m), 7.63 (1H, d, J=1.7 Hz), 7.48 (1H, dd, J=7.9, 1.8 Hz), 7.06 (1H, d, J=8.1 Hz), 6.26 (2H, s), 2.10 (3H, s). MS ESI (pos.) m/e: 360.9 (M+H).

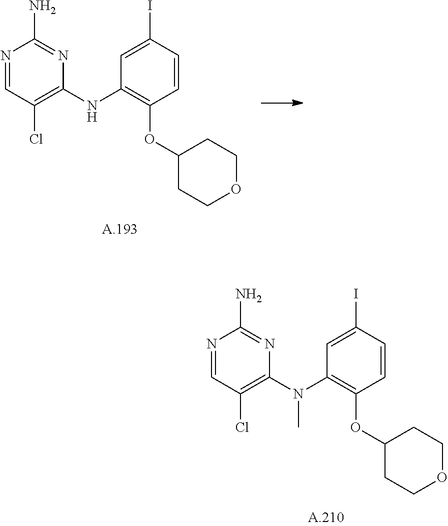

5-Chloro-N⁴-(5-iodo-2-(tetrahydro-2H-pyran-4-yloxy)phenyl)-N⁴-methylpyrimidine-2,4-diamine (A.210). A dry flask containing 5-chloro-N⁴-(5-iodo-2-(tetrahydro-2H-pyran-4-yloxy)phenyl)pyrimidine-2,4-diamine (A.193) (447.2 mg, 1.0 mmol) in dry DMF (8.00 mL) was cooled in an ice bath, then sodium hydride (60% wt. dispersion in oil) (40.9 mg, 1.02 mmol) was carefully added in portions. After 15 minutes, methyl iodide (0.056 mL, 0.90 mmol) was added, and the mixture was stirred at room temperature. After 1 hour, the reaction was carefully quenched with cold water then extracted three times with ethyl acetate. After concentration, the residue was purified by silica gel flash chromatography (50:1:1 solution of dichloromethane: methanol: 2M ammonia in methanol) to afford 5-chloro-N⁴-(5-iodo-2-(tetrahydro-2H-pyran-4-yloxy)phenyl)-N⁴-methylpyrimidine-2,4-diamine (A.210) (268.6 mg, 58.3%). MS ESI (pos.) m/e: 461.0 (M+H).

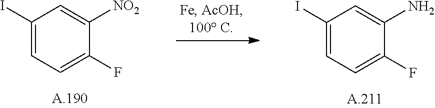

2-Fluoro-5-iodobenzenamine (A.211). To a flask containing 1-fluoro-4-iodo-2-nitrobenzene A.190 (717.1 mg, 2.69 mmol) and acetic acid (10.00 mL) was added iron powder (600.3 mg, 10.7 mmol). The reaction was carefully heated to 100° C. After 1 hour, the reaction was cooled to room temperature then filtered through celite. After rinsing the filter cake three times with ethanol, the organic solvent was removed under reduced pressure to afford 2-fluoro-5-iodobenzenamine (A.211) (609.7 mg, 96%). ¹H NMR (500 MHz, DMSO-d₆) δ ppm 7.10 (1H, m), 6.80 (2H, m), 5.35 (2H, s). MS ESI (pos.) m/e: 237.9 (M+H).

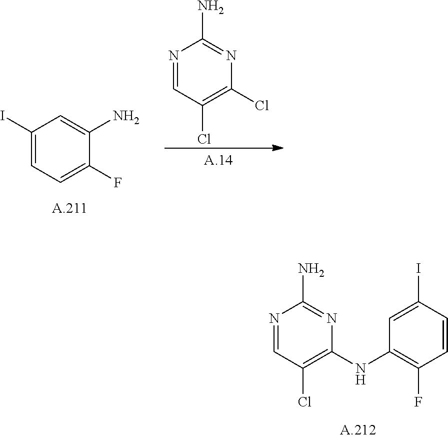

5-Chloro-N⁴-(2-fluoro-5-iodophenyl)pyrimidine-2,4-diamine (A.212). To a flask containing 2-fluoro-5-iodobenzenamine (A.211) (609.7 mg, 2.57 mmol) in 1,4-dioxane (5.00 mL) was added 4,5-dichloropyrimidin-2-amine (A.14)(844.0 mg, 5.15 mmol) and 1 N HCl (5.00 mL). The reaction was heated to 80° C. After 3 hours, the mixture was cooled to room temperature then carefully neutralized with 1N NaOH. After extracting with dichloromethane, the solvent was removed under reduced pressure. Ethyl acetate was then added to the residue and heated to 50° C. After 15 minutes, the solvent was removed to a volume of ˜1.0 mL and cooled to room temperature. The solid was filtered and dried to yield 5-chloro-N⁴-(2-fluoro-5-iodophenyl)pyrimidine-2,4-diamine (A.212) (402.8 mg, 43%). ¹H NMR (500 MHz, DMSO-d₆) δ ppm 8.45 (1H, s), 7.92 (1H, m), 7.86 (1H, dd, J=7.1, 2.2 Hz), 7.56 (1H, ddd, J=8.5, 4.5, 2.2 Hz), 7.10 (1H, dd, J=10.6, 8.7 Hz), 6.35 (2H, s). MS ESI (pos.) m/e: 364.9

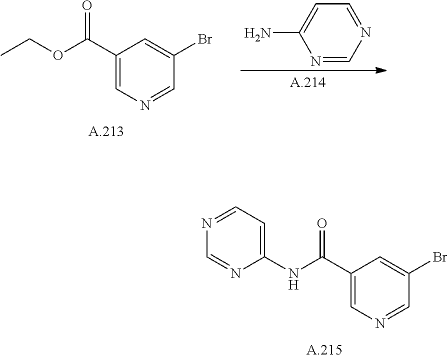

5-Bromo-N-(pyrimidin-4-yl)nicotinamide (A.215). A dry flask containing pyrimidin-4-amine A.214 (208.9 mg, 2.2 mmol) in dry DMF (8.00 mL) was cooled in an ice bath, then potassium tert-butoxide (370.2 mg, 3.3 mmol) was carefully added in portions. After 15 minutes, ethyl 5-bromonicotinate A.213 (759.7 mg, 3.3 mmol) was added, and the mixture was heated to 50° C. After 1 hour, the reaction was carefully quenched with cold water then extracted three times with dichloromethane. After concentrating under reduced pressure, isopropanol was added to the residue and heated to 50° C. After 15 minutes, the solvent was removed to a volume of ˜1.0 mL and cooled to room temperature. The solid was filtered and dried to yield 5-bromo-N-(pyrimidin-4-yl)nicotinamide (A.215)(200 mg, 33%). ¹H NMR (500 MHz, DMSO-d₆) δ ppm 11.57 (1H, s), 9.08 (1H, d, J=2.0 Hz), 8.98 (1H, s), 8.93 (1H, d, J=2.2 Hz), 8.76 (1H, d, J=5.6 Hz), 8.60 (1H, t, J=2.1 Hz), 8.19 (1H, d, J=5.9 Hz). MS ESI (pos.) m/e: 279.0 (M+H).

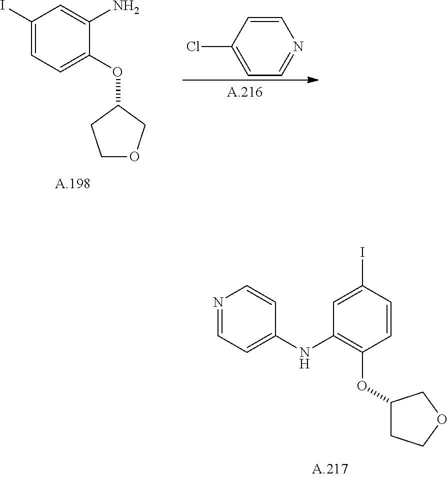

(S)-N-(5-iodo-2-(tetrahydrofuran-3-yloxy)phenyl)pyridin-4-amine (A.217). To a flask containing (S)-5-iodo-2-(tetrahydrofuran-3-yloxy)benzenamine (A.198) (396.9 mg, 1.30 mmol) in pentanol (8.00 mL) was added 4-chloropyridine (A.216)(390.8 mg, 2.6 mmol). The reaction was heated to 140° C. After 17 hours, the mixture was cooled to room temperature then carefully neutralized with 1N NaOH. After extracting with dichloromethane, the residue was purified by silica gel flash chromatography (30:1:1 solution of dichloromethane: methanol: 2M ammonia in methanol) to afford (S)-N-(5-iodo-2-(tetrahydrofuran-3-yloxy)phenyl)pyridin-4-amine (A.217) (298.6 mg, 60%). ¹H NMR (500 MHz, DMSO-d₆) δ ppm 8.15 (3H, d, J=3.7 Hz), 7.51 (1H, d, J=2.0 Hz), 7.39 (1H, dd, J=8.6, 2.0 Hz), 6.88 (1H, d, J=8.6 Hz), 6.73 (2H, d, J=6.1 Hz), 4.99 (1H, t, J=5.3 Hz), 3.86 (1H, dd, J=10.0, 4.6 Hz), 3.74 (3H, m), 2.19 (1H, m), 1.94 (1H, m). MS ESI (pos.) m/e: 382.9 (M+H).

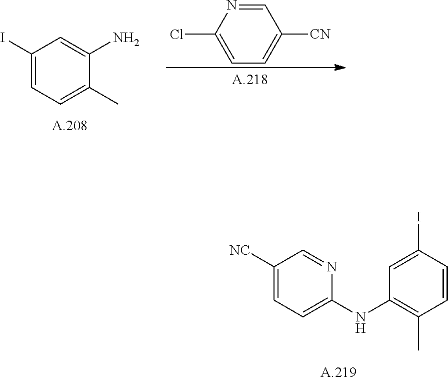

6-(5-Iodo-2-methylphenylamino)nicotinonitrile (A.219). To a flask containing 5-iodo-2-methylbenzenamine (A.208)(1.01 g, 4.32 mmol) in pentanol (16.00 mL) was added 6-chloronicotinonitrile (A.218) (1.20 g, 8.64 mmol) and 1 drop of concentrated hydrochloric acid. The reaction was heated to 140° C. After 37 hours, the mixture was cooled to room temperature then carefully neutralized with 1N NaOH. After extracting with dichloromethane, the residue was purified by silica gel flash chromatography (3:1 hexanes:ethyl acetate) to afford 6-(5-iodo-2-methylphenylamino)nicotinonitrile (A.219) (907.8 mg, 63%). ¹H NMR (500 MHz, DMSO-d₆) δ ppm 9.13 (1H, s), 8.49 (1H, s), 7.82-7.90 (2H, m), 7.45 (1H, d, J=7.8 Hz), 7.07 (1H, d, J=7.8 Hz), 6.81 (1H, d, J=8.8 Hz), 2.16 (3H, s). MS ESI (pos.) m/e: 336.0 (M+H).

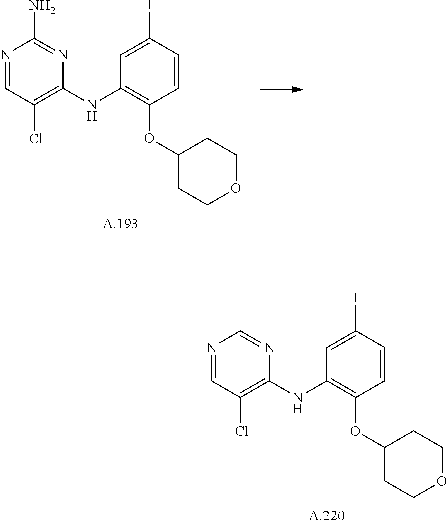

### 5-Chloro-N-(5-iodo-2-(tetrahydro-2H-pyran-4-yloxy)phenyl)pyrimidin-4-amine (A.220)

To a flask containing 5-chloro-N⁴-(5-iodo-2-(tetrahydro-2H-pyran-4-yloxy)phenyl)pyrimidine-2,4-diamine (A.193) (500.2 mg, 1.12 mmol) in THF (5.00 mL) was added isoamyl nitrite (0.7 mL, 5.23 mmol) dropwise over 5 minutes. The reaction was heated to 70° C. After 8 hours, the mixture was cooled to room temperature then carefully poured into ice water. After extracting with dichloromethane, the residue was concentrated then purified by silica gel flash chromatography (2:1 dichloromethane:ethyl acetate) to afford 5-chloro-N-(5-iodo-2-(tetrahydro-2H-pyran-4-yloxy)phenyl)pyrimidin-4-amine (A.220) (126.4 mg, 26%). ¹H NMR (500 MHz, DMSO-d₆) δ ppm 8.60 (1H, s), 8.54 (1H, s), 8.44 (2H, s), 7.45 (1H, dd, J=8.6, 2.2 Hz), 7.04 (1H, d, J=8.6 Hz), 4.68 (1H, ddd, J=7.7, 3.8, 3.7 Hz), 3.79 (2H, m), 3.48 (2H, ddd, J=11.4, 8.1, 3.2 Hz), 1.99 (2H, m), 1.62 (2H, m). MS ESI (pos.) m/e: 432.1 (M+H).

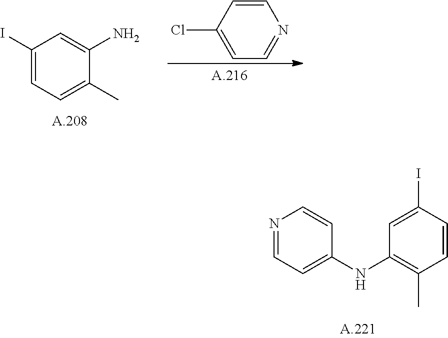

5-Chloro-N-(5-iodo-2-(tetrahydro-2H-pyran-4-yloxy)phenyl)pyrimidin-4-amine (A.221). To a flask containing 5-iodo-2-methylbenzenamine (A.208)(502.8 mg, 2.16 mmol) in pentanol (10.00 mL) was added 4-chloropyridine (A.216)(647.5 mg, 4.32 mmol). The reaction was heated to 140° C. After 4.5 days, the mixture was cooled to room temperature then filtered The filtrate was concentrated then purified by silica gel flash chromatography (30:1:1 solution of dichloromethane: methanol: 2M ammonia in methanol) to afford N-(5-iodo-2-methylphenyl)pyridin-4-amine (A.221) (499 mg, 74%). ¹H NMR (500 MHz, DMSO-d₆) δ ppm 8.36 (1H, s), 8.18 (2H, m), 7.53 (1H, d, J=1.7 Hz), 7.44 (1H, dd, J=7.8, 1.7 Hz), 7.09 (1 H, d, J=7.8 Hz), 6.64 (2H, dd, J=4.8, 1.6 Hz), 2.13 (3H, s).

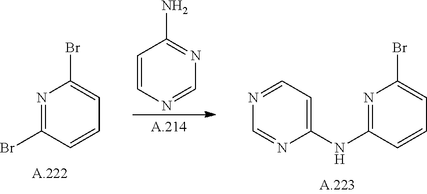

### 5-Chloro-N-(5-iodo-2-(tetrahydro-2H-pyran-4-yloxy)phenyl)pyrimidin-4-amine (A.223)

To a dry flask was added 2,6-dibromopyridine (A.222)(1.04 g, 4.41 mmol), pyrimidin-4-amine (A.214)(419.1 mg, 4.41 mmol), Pd₂(dba)₃ (80.9 mg, 0.083 mmol), Xantphos (153.3 mg, 0.27 mmol), cesium carbonate (2.01 g, 6.17 mmol), and dry dioxane (15.0 mL). After evacuating and backfilling with argon, the mixture was heated to 80° C. After 18 hours, the reaction was cooled to room temperature then filtered through celite. After rinsing the celite three times with THF, the filtrate was concentrated under reduced pressure. The residue was purified by silica gel flash chromatography (100% ethyl acetate) to afford N-(6-bromopyridin-2-yl)pyrimidin-4-amine (A.223) 510.7 mg, 46%). MS ESI (pos.) m/e: 251.0 (M+H).

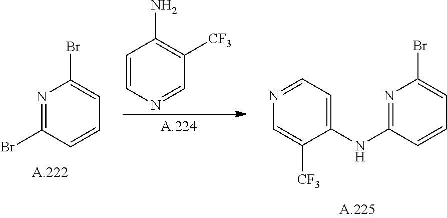

6-Bromo-N-(3-(trifluoromethyl)pyridin-4-yl)pyridin-2-amine (A.225). To a dry flask was added 2,6-dibromopyridine (A.222)(882.2 mg, 3.72 mmol), 3-(trifluoromethyl)pyridin-4-amine (A.224)(503.0 mg, 3.1 mmol), Pd₂(dba)₃ (57.7 mg, 0.063 mmol), Xantphos (108.4 mg, 0.19 mmol), cesium carbonate (1.41 g, 6.17 mmol), and dry dioxane (8 mL). After evacuating and backfilling with argon, the mixture was heated to 80° C. After 19 hours, the reaction was cooled to room temperature then filtered through celite. After rinsing the celite three times with THF, the filtrate was concentrated under reduced pressure. The residue was purified by silica gel flash chromatography (80% ethyl acetate in hexanes) to afford 6-bromo-N-(3-(trifluoromethyl)pyridin-4-yl)pyridin-2-amine (A.225) (708.9 mg, 72%). ¹H NMR (500 MHz, DMSO-d₆) δ ppm 8.88 (1H, s), 8.74 (1H, s), 8.61 (1H, d, J=5.6 Hz), 7.96 (1H, d, J=5.9 Hz), 7.67 (1H, t, J=7.8 Hz), 7.29 (1H, d, J=8.1 Hz), 7.23 (1H, d, J=7.6 Hz). MS ESI (pos.) m/e: 318.0 (M+H).

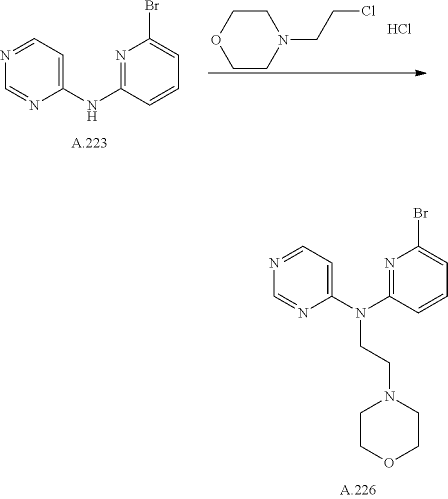

N-(6-Bromopyridin-2-yl)-N-(2-morpholinoethyl)pyrimidin-4-amine (A.226). A dry flask containing N-(6-bromopyridin-2-yl)pyrimidin-4-amine (A.223) (311.3 mL, 1.24 mmol) in dry DMF (4.00 mL) was cooled in an ice bath, then sodium hydride (60% wt. dispersion in oil) (61.1 mg, 1.53 mmol) was carefully added in portions. After 15 minutes, 4-(2-chloroethyl)morpholine hydrochloride in dichloromethane:pyridine (3:1) (277.4 mg, 1.49 mmol) was added dropwise, and the mixture was heated to 80° C. After 21 hours, the reaction was carefully quenched with cold water then extracted three times with dichloromethane. After removing the organic solvent under reduced pressure, the residue was purified by silica gel flash chromatography (35:1:1 solution of dichloromethane: methanol: 2M ammonia in methanol) to afford N-(6-bromopyridin-2-yl)-N-(2-morpholinoethyl)pyrimidin-4-amine (A.226) (168.9 mg, 37%). ¹H NMR (500 MHz, MeOH) δ ppm 8.68 (1H, s), 8.25 (1H, d, J=6.4 Hz), 7.71 (1H, t, J=7.8 Hz), 7.48 (2H, m), 6.99 (1H, dd, J=6.2, 1.3 Hz), 4.35 (2H, t, J=6.6 Hz), 3.56 (4H, m), 2.69 (2H, t, J=6.7 Hz), 2.47 (4H, m). MS ESI (pos.) m/e: 364.0 (M+H).

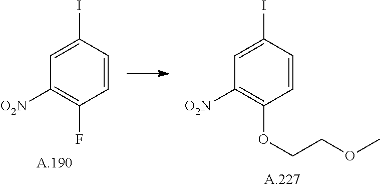

### 4-iodo-1-(2-methoxyethoxy)-2-nitrobenzene (A.227)

To a solution of 2-methoxyethanol (1.93 mL, 24.4 mmol) in DMF (24 mL) was added NaH (60% dispersion in mineral oil, 0.5865 g, 24.4 mmol) and the mixture was stirred at 0° C. for 1.5 hours. To the mixture was added a solution of 1-fluoro-4-iodo-2-nitrobenzene A.190 (5 g, 18.8 mmol) in DMF (6 mL) at 0° C. and the mixture was allowed to warm to room temperature with stirring. After stirring at room temperature for 21 hours, the mixture was poured into ice water (200 mL) and extracted with dichloromethane (100 mL×3). The combined organic layers were washed with brine (150 mL×1), dried over Na₂SO₄, filtered, and concentrated under reduced pressure to give an orange oil. The product was purified by silica gel column chromatography using 0% to 50% gradient of ethyl acetate in hexane as eluent to give 4-iodo-1-(2-methoxyethoxy)-2-nitrobenzene (A.227) (4.67 g, 77.2% yield) as an orange oil: ¹H NMR (500 MHz, DMSO-d₆) δ ppm 8.16 (1H, d, J=2.2 Hz), 7.94 (1H, dd, J=8.9, 2.3 Hz), 7.21 (1H, d, J=8.8 Hz), 4.27 (2H, dd, J=5.3, 3.8 Hz), 3.65 (2H, dd, J=5.0, 3.8 Hz), 3.29 (3H, s); Mass Spectrum (ESI) m/e=324.0 [M+1].

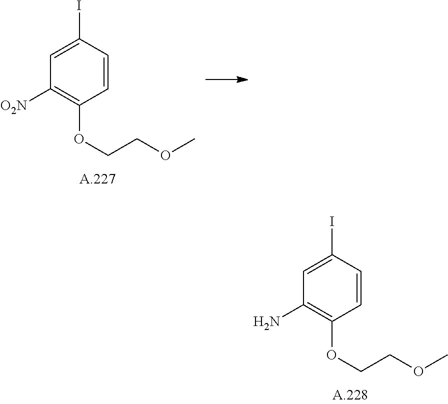

5-iodo-2-(2-methoxyethoxy)benzenamine (A.228): To a solution of 4-iodo-1-(2-methoxyethoxy)-2-nitrobenzene (A.227) (4.67 g, 14.45 mmol) in ethyl acetate (120 mL) was added SnCl₂.2H₂O (16.31 g, 72.27 mmol) and the mixture was heated at reflux for 5.5 hours. The mixture was cooled to room temperature. To the mixture was added 1 N aqueous HCl (140 mL, 140 mmol). The mixture was basified to pH 10.0 with 10 N aqueous NaOH and extracted with ethyl acetate (100 mL×2). The combined organic layers were washed with brine (100 mL×1), dried over MgSO₄, filtered, and concentrated under reduced pressure to give an orange oil. The product was purified by silica gel column chromatography using 10% to 100% gradient of ethyl acetate in hexane as eluent to give 5-iodo-2-(2-methoxyethoxy)benzenamine (A.228) (3.4984 g, 82.6% yield) as a yellow oil: ¹H NMR (500 MHz, DMSO-d₆) δ ppm 6.95 (1H, d, J=2.2 Hz), 6.71-6.79 (1H, m), 6.61 (1H, d, J=8.3 Hz), 4.83-4.92 (2H, m), 4.03 (2H, dd, J=5.5, 3.8 Hz), 3.58-3.67 (2H, m), 3.25-3.33 (3H, m);

Mass Spectrum (ESI) m/e=294.0 [M+1].

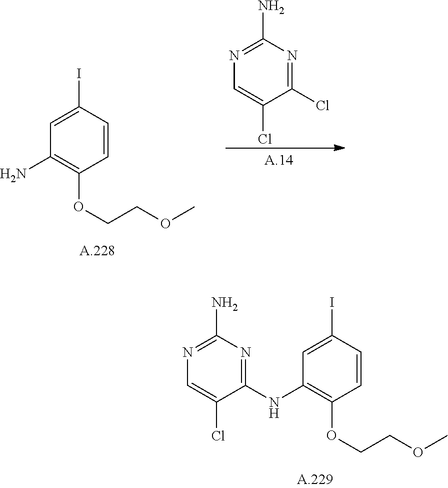

5-chloro-N4-(5-iodo-2-(2-methoxyethoxy)phenyl)pyrimidine-2,4-diamine (A.229): A mixture of 5-iodo-2-(2-methoxyethoxy)benzenamine (A.228)(3 g, 10.24 mmol) and 4,5-dichloropyrimidin-2-amine (A.14)(1.679 g, 10.24 mmol) in 1,4-dioxane (25 mL) was stirred at 90° C. for 23 hours. The mixture was cooled to room temperature and concentrated under reduced pressure. The residue was suspended in NH₄OH (100 mL). The resulting precipitate was collected by suction filtration, washed with water (200 mL), and dried to give a tan solid. The tan solid was purified by silica gel column chromatography using 10% to 20% gradient of dichloromethane-methanol-NH₄OH (89:9:1) in dichloromethane as eluent to give a tan solid. The tan solid was washed with dichloromethane-methanol (1:1) and filtered to give 5-chloro-N4-(5-iodo-2-(2-methoxyethoxy)phenyl)pyrimidine-2,4-diamine (A.229) (2.31 g, 53.7% yield) as a light orange solid: ¹H NMR (500 MHz, DMSO-d₆) δ ppm 8.66 (1H, d, J=2.0 Hz), 7.86-8.02 (2H, m), 7.28-7.41 (1H, m), 6.93 (1H, d, J=8.6 Hz), 6.64 (2H, s), 4.13-4.23 (2H, m), 3.59-3.70 (2H, m), 3.30 (3H, s); Mass Spectrum (ESI) m/e=421.0 [M+1].

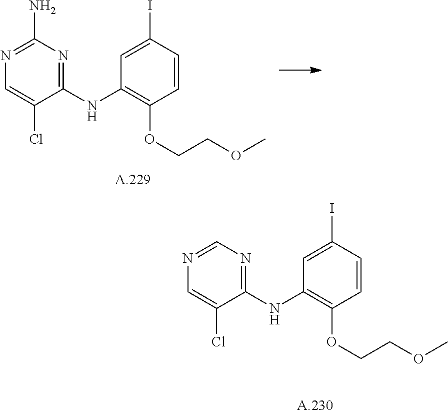

5-chloro-N-(5-iodo-2-(2-methoxyethoxy)phenyl)pyrimidin-4-amine (A.230): To a suspension of 5-chloro-N4-(5-iodo-2-(2-methoxyethoxy)phenyl)pyrimidine-2,4-diamine (A.229) (0.5 g, 1.19 mmol) in THF (1 mL) was added dropwise isoamylnitrite (0.75 mL, 5.6 mmol) with stirring and the mixture was heated at reflux for 7 hours. The mixture was poured into ice water (50 mL) and extracted with ethyl acetate (50 mL×3). The combined organic layers were washed with brine (100 mL×1), dried over MgSO₄, filtered, and concentrated under reduced pressure to give an orange syrupy solid. The product was purified by silica gel column chromatography using 0% to 100% gradient of ethyl acetate in hexane as eluent to give 5-chloro-N-(5-iodo-2-(2-methoxyethoxy)phenyl)pyrimidin-4-amine (A.230) (0.148 g, 30.6% yield): ¹H NMR (500 MHz, DMSO-d₆) δ ppm 8.61 (1H, s), 8.53 (1H, s), 8.44 (1H, d, J=2.2 Hz), 8.42 (1H, s), 7.46 (1H, dd, J=8.4, 1.8 Hz), 6.99 (1H, d, J=8.6 Hz), 4.18 (2H, dd, J=5.4, 3.7 Hz), 3.61 (2H, dd, J=5.4, 3.7 Hz), 3.26 (3H, s); Mass Spectrum (ESI) m/e=406.0 [M+1].

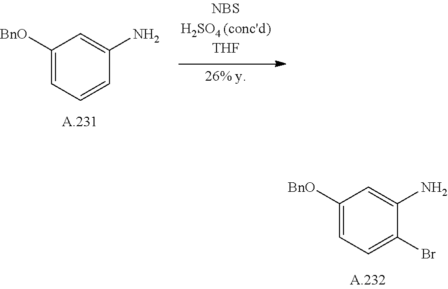

### 5-(Benzyloxy)-2-bromoaniline A.232

Ref. Boger, D. L., R. J. Wysocki, et al. (1990). J. Amer. Chem. Soc. 112(13): 5230-5240.

A solution of 3-(benzyloxy)aniline A.231 (50 mmol, 10.0 g) in THF (100 mL) was stirred in an acetone-dry ice bath. To this solution was added a few drops of concentrated H₂SO₄. The mixture was stirred in the dry ice bath for 5 min before NBS (1.0 eq, 9.0 g) was added in one-gram portions over a period of 30 min. The resulting mixture was stirred in the acetone-dry ice bath for 1 h 15 min. Na₂CO₃ (1.5 g) was added and the dry ice bath was removed. The reaction mixture was allowed to warm up to room temperature, poured into ice and saturated Na₂CO₃ aqueous solution, and extracted with ethyl acetate (2×). The combined organics were washed with brine (1×), dried over Na₂SO₄, and concentrated in vacuo. The residue was subjected to combi-flash column chromatography (ethyl acetate/hexanes) to give 5-(benzyloxy)-2-bromoaniline A.232 (3.7 g, 26% yield) as a white solid. ¹H NMR (500 MHz, CHLOROFORM-d) δ ppm 7.36-7.43 (4H, m), 7.31-7.35 (1H, m), 7.27-7.30 (1H, m), 6.42 (1H, d, J=2.7 Hz), 6.31 (1H, dd, J=8.8, 2.7 Hz), 5.01 (2H, s). LCMS-ESI (POS), M/Z, M+1: Found 278.0 and 280.0.

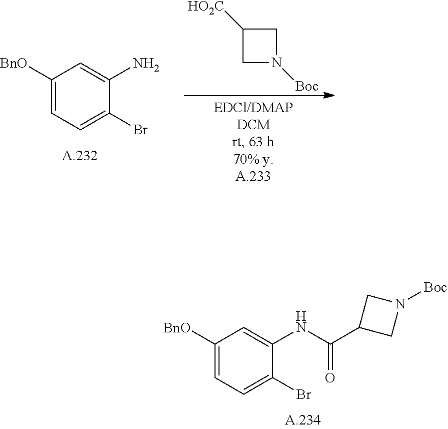

### 3-((5-(Benzyloxy)-2-bromophenyl)carbamoyl)-1-azetidinecarboxylate

Ref. Helv. Chim. Acta 2000, 83, 1247-55.

To a stirred solution of 5-(benzyloxy)-2-bromoaniline A.232 (3.7 g, 13.1 mmol) and DMAP (1.3 eq, 2.1 g) in dichloromethane (90 mL) was added 1-(tert-butoxycarbonyl)-3-azetidinecarboxylic acid A.233 (1.0 eq, 2.7 g) in one portion followed by one-portion addition of EDCI (1.3 eq, 3.3 g) at room temperature. The resulting mixture was stirred at rt for 63 h. After the solvent was removed, water was added to the residue followed by extraction with ethyl acetate (2×). The combined organics were washed with ice cold 2 N HCl aqueous solution (1×), water (1×), saturated Na₂CO₃ aqueous solution (1×), brine (1×), dried over Na₂SO₄, and concentrated in vacuo to give 3-((5-(benzyloxy)-2-bromophenyl)carbamoyl)-1-azetidinecarboxylate A.234. (4.2 g, 70% yield) as a sticky oil. LCMS-ESI (POS), M/Z, M+1: Found 361.0 and 363.0; 405.0 and 407.0; 483.0 and 485.0 (Na⁺).

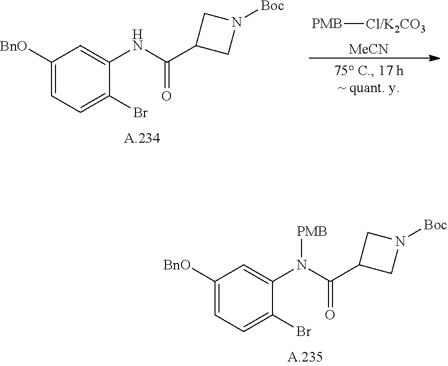

### tert-Butyl 3-((5-(benzyloxy)-2-bromophenyl)(4-methoxybenzyl)carbamoyl)-1-azetidinecarboxylate A.235

A mixture of 3-((5-(benzyloxy)-2-bromophenyl)carbamoyl)-1-azetidinecarboxylate A.234 (4.2 g, 9.1 mmol), p-methoxybenzyl chloride (1.1 eq, 1.6 g), and K₂CO₃ (3.0 eq, 3.8 g) in acetonitrile (120 mL) was refluxed for 17 h. After cooled to room temperature, the mixture was vacuum filtered through a layer of celite and the filter cake was washed with more acetonitrile and ethyl acetate. The filtrate was concentrated in vacuo and the residue was subjected to combi-flash column chromatography (ethyl acetate/hexane) to give tert-butyl 3-((5-(benzyloxy)-2-bromophenyl) (4-methoxybenzyl)carbamoyl)-1-azetidinecarboxylate A.235 (5.5 g, 100% yield) as a colorless sticky liquid. LCMS-ESI (POS), M/Z, M+1: Found 581.2 and 583.2; 525.0 and 527.0; 481.1 and 483.1.

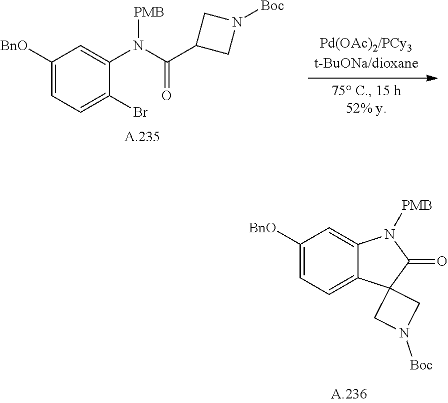

tert-butyl 6′-(benzyloxy)-1′-(4-methoxybenzyl)-2′-oxospiro[azetidine-3,3′-indoline]-1-carboxylate (A.236) was prepared according to the procedure reported in Lee, S, and J. F. Hartwig (2001). J. Org. Chem. 66(10): 3402-3415.

To a 200 mL single-necked round bottom flask were placed tert-butyl 3-((5-(benzyloxy)-2-bromophenyl) (4-methoxybenzyl)carbamoyl)-1-azetidinecarboxylate A.235 (5.5 g, 9.5 mmol), sodium tert-butoxide (1.5 eq, 1.4 g) and palladium (II) acetate (10 mol %, 0.21 g). The flask was subjected to 3 cycles of evacuation and back-filling with N₂. 1,4-dioxane was added followed by tricyclohexylphosphine (10 mol %, 0.27 g). The resulting mixture was stirred at 75° C. for 15 h. After cooling, the mixture was poured into ice and saturated NH₄Cl aqueous solution and extracted with ethyl acetate (2×). The combined organics were washed with brine (1×), dried over Na₂SO₄, and concentrated in vacuo. The residue was subjected to combi-flash column chromatography (ethyl acetate/hexanes) to give tert-butyl 6′-(benzyloxy)-1′-(4-methoxybenzyl)-2′-oxo-1H-spiro[azetidine-3,3′-indoline]-1-carboxylate A.236 (2.4 g, 52% yield) as a sticky liquid. ¹H NMR (400 MHz, CHLOROFORM-d) δ ppm 7.31-7.44 (6H, m), 7.19 (2H, d, J=8.6 Hz), 6.83 (2H, d, J=9.0 Hz), 6.67 (1H, dd, J=8.2, 2.3 Hz), 6.43 (1H, d, J=2.3 Hz), 5.02 (2H, s), 4.78 (2H, s), 4.39 (2H, d, J=8.2 Hz), 4.05 (2H, d, J=8.2 Hz), 3.78 (3 H, s), 1.50 (9H, s). LCMS-ESI (POS), M/Z, M+1: Found 445.1; 523.2 (Na⁺).

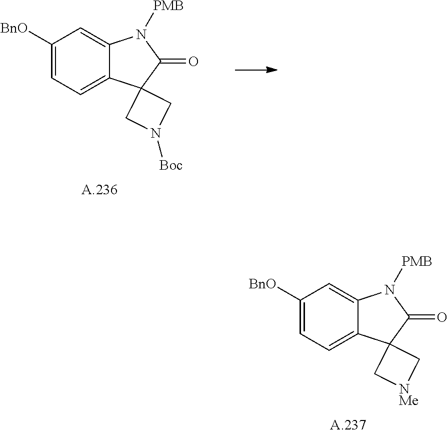

### 6′-(Benzyloxy)-1′-(4-methoxybenzyl)-1-methyl-1′,2′-dihydrospiro[azetidine-3,3′-indole] A.237

To a stirred solution of tert-butyl 6′-(benzyloxy)-1′-(4-methoxybenzyl)-2′-oxo-1H-spiro[azetidine-3,3′-indoline]-1-carboxylate A.236 (2.4 g, 4.8 mmol) in dry toluene (80 mL), which was preheated at 80° C. in an oil bath, was dropwise added a solution of Red-AL in toluene (3 M) (9.5 mL, 6.0 eq.) under a nitrogen atmosphere. The resulting mixture was stirred at this temperature for 2 h. Upon workup, the mixture was cooled in an ice-salt bath before cautiously quenched with ice-cold aqueous 2 N NaOH. The mixture was further diluted with ice-cold aqueous 2 N NaOH and extracted with ethyl acetate (3×). The combined organics were dried over anhydrous Na₂SO₄ and concentrated in vacuo to give 6′-(benzyloxy)-1′-(4-methoxybenzyl)-1-methylspiro[azetidine-3,3′-indoline] (A.237)(1.9 g, 100% yield) as a sticky liquid. ¹H NMR (400 MHz, DMSO-d₆) δ ppm 7.29-7.45 (5H, m), 7.19-7.24 (3H, m), 6.83-6.89 (2H, m), 6.35 (1H, dd, J=8.0, 2.2 Hz), 6.19 (1H, d, J=2.3 Hz), 5.02 (2H, s), 4.18 (2H, s), 3.81 (3H, s), 3.55 (2H, s), 3.40-3.45 (2H, m), 3.26-3.32 (1H, m). LCMS-ESI (POS), M/Z, M+1: Found 401.2.

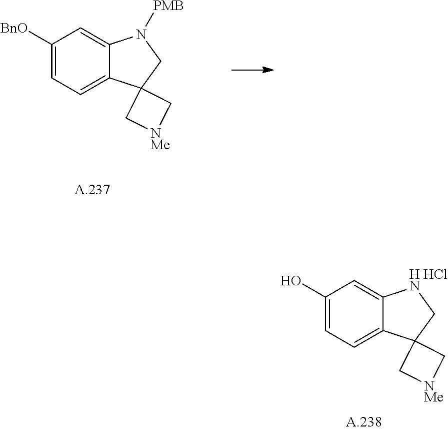

### 1-methylspiro[azetidine-3,3′-indolin]-6′-ol hydrochloride (A.238)

A mixture of 6′-(benzyloxy)-1′-(4-methoxybenzyl)-1-methylspiro[azetidine-3,3′-indoline] A.237 (1.0 g, 2.5 mmol), Pd/C (10 wt % on activated C, wet) (not weighed), and 37% HCl aqueous solution (commercial) (3 mL) in EtOH (40 mL) was balloon hydrogenated for 15 h. Upon workup, water was added and the mixture was vacuum filtered through a layer of celite. The filter cake was washed with EtOH/water (8:1, v/v). The filtrate was concentrated in vacuo to give 1-methylspiro[azetidine-3,3′-indolin]-6′-ol hydrochloride, A.238 which was used directly in the next step. LCMS-ESI (POS), M/Z, M+1: Found 191.1.

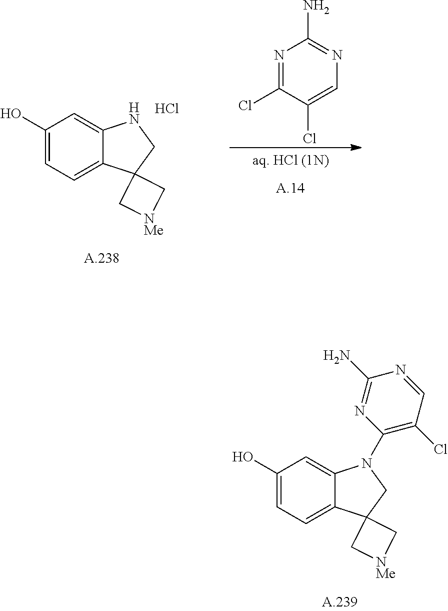

### 1′-(2-amino-5-chloropyrimidin-4-yl)-1-methylspiro[azetidine-3,3′-indolin]-6′-ol A.239

A mixture of 1-methylspiro[azetidine-3,3′-indolin]-6′-ol hydrochloride A.238 (2.5 mmol) (assuming 100% yield in the previous step) and 4,5-dichloro-2-pyrimidinamine A.14 (1.2 eq., 0.5 g) in 1N HCl aqueous solution was stirred at 60° C. for 21 h. Upon workup, the reaction mixture was cooled to room temperature and the precipitate was collected by vacuum filtration. The filter cake was washed with a small volume of ice cold 1N HCl aqueous solution and dried under high vacuum to give 1′-(2-amino-5-chloropyrimidin-4-yl)-1-methylspiro[azetidine-3,3′-indolin]-6′-ol HCl salt A.239 (0.75 g, 85% over 2 steps) as an off-white solid. LCMS-ESI (POS), M/Z, M+1: Found 318.1.

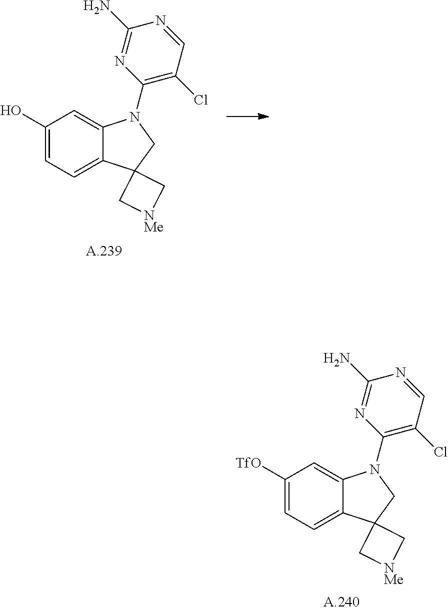

### 1′-(2-amino-5-chloropyrimidin-4-yl)-1-methylspiro[azetidine-3,3′-indoline]-6′-yl trifluoromethanesulfonate A.240

To a stirred mixture of 1′-(2-amino-5-chloropyrimidin-4-yl)-1-methylspiro[azetidine-3,3′-indolin]-6′-ol HCl salt A.239 (0.65 g, 2.0 mmol) and triethylamine (4.0 eq., 1.1 mL) in dichloromethane (20 mL) was added 1,1,1-trifluoro-N-phenyl-N-((trifluoromethyl)sulfonyl)methanesulfonamide (1.5 eq., 0.9 g) at room temperature. The resulting mixture was stirred for 24 h. Upon workup, the crude reaction mixture was subjected to combi-flash column chromatography (methanol/dichloromethane with triethylamine) to give 1′-(2-amino-5-chloropyrimidin-4-yl)-1-methylspiro[azetidine-3,3′-indoline]-6′-yl trifluoromethanesulfonate A.240 (0.5 g, 60% yield) as an off-white solid. ¹H NMR (400 MHz, DMSO-d₆) δ ppm 8.13 (1H, s), 7.71 (1H, d, J=8.2 Hz), 7.52 (1H, d, J=2.3 Hz), 7.06 (1H, dd, J=8.2, 2.3 Hz), 6.67 (2H, s), 4.49 (2H, s), 3.36-3.41 (2H, m), 3.31-3.34 (2H, m), 2.29 (3 H, s). LCMS-ESI (POS), M/Z, M+1: Found 450.0.

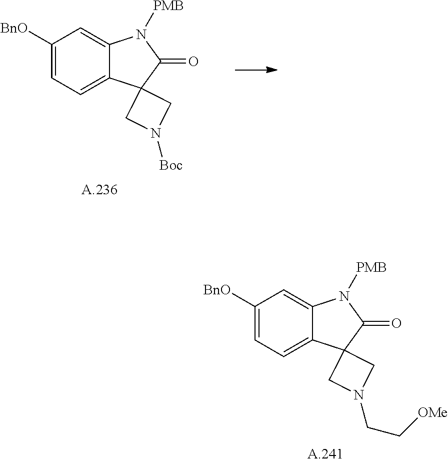

### 6′-(Benzyloxy)-1′-(4-methoxybenzyl)-1-(2-methoxyethyl)spiro[azetidine-3,3′-indolin]-2′-one A.241

To a stirred solution of tert-butyl 6′-(benzyloxy)-1′-(4-methoxybenzyl)-2′-oxo-1H-spiro[azetidine-3,3′-indoline]-1-carboxylate A.236 (0.7 g, 1.40 mmol) in dichloromethane (10 mL) was added TFA (1.0 mL) at room temperature. The resulting mixture was stirred at room temperature for 3 h. The volatiles were removed in vacuo and the residue was dissolved in DMF (15 mL). To this solution were added Cs₂CO₃ (3.0 eq., 1.36 g) and 2-bromoethyl methyl ether (1.0 eq., 0.14 mL) sequentially at room temperature. The resulting mixture was stirred at room temperature for 3 days. Upon workup, the mixture was poured into ice and 2 N NaOH aqueous solution and extracted with ethyl acetate (2×). The combined organics were washed with ice cold 2 N NaOH aqueous solution (1×) and dried over Na₂SO₄. The residue after concentration in vacuo was subjected to combi-flash column chromatography (methanol/dichloromethane with triethylamine) to give 6′-(benzyloxy)-1′-(4-methoxybenzyl)-1-(2-methoxyethyl)spiro[azetidine-3,3′-indolin]-2′-one A.241 (0.23 g, 36% yield in 2 steps) as a sticky liquid. ¹H NMR (400 MHz, CHLOROFORM-d) δ ppm 7.65 (1H, d, J=7.8 Hz), 7.29-7.44 (5H, m), 7.19 (2H, d, J=8.6 Hz), 6.82 (2H, d, J=8.6 Hz), 6.67 (1H, dd, J=8.0, 2.2 Hz), 6.41 (1H, d, J=2.3 Hz), 5.01 (2H, s), 4.77 (2H, s), 3.77 (3H, s), 3.65-3.72 (2H, m), 3.61 (2 H, d, J=7.0 Hz), 3.49 (2H, t, J=5.5 Hz), 3.38 (3H, s), 2.85 (2H, t, J=5.7 Hz). LCMS-ESI (POS), M/Z, M+1: Found 459.2.

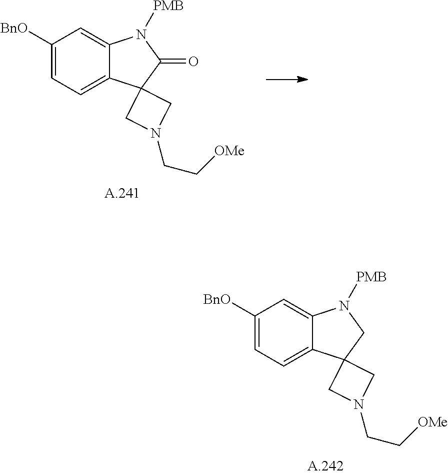

6′-(benzyloxy)-1′-(4-methoxybenzyl)-1-(2-methoxyethyl)spiro[azetidine-3,3′-indoline] A.242 was prepared from 6′-(benzyloxy)-1′-(4-methoxybenzyl)-1-(2-methoxyethyl)spiro[azetidine-3,3′-indolin]-2′-one A.241 using chemistry similar to that described for compound A.237. LCMS-ESI (POS), M/Z, M+1: Found 445.1.

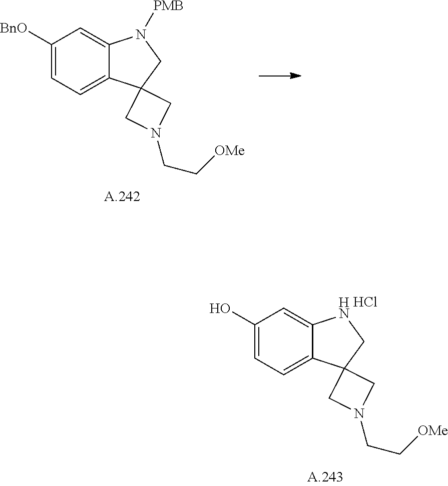

1-(2-methoxyethyl)spiro[azetidine-3,3′-indolin]-6′-ol hydrochloride A.243 was prepared from 6′-(benzyloxy)-1′-(4-methoxybenzyl)-1-(2-methoxyethyl)spiro[azetidine-3,3′-indoline]A.242 using chemistry similar to that described for compound A.238. LCMS-ESI (POS), M/Z, M+1: Found 235.1.

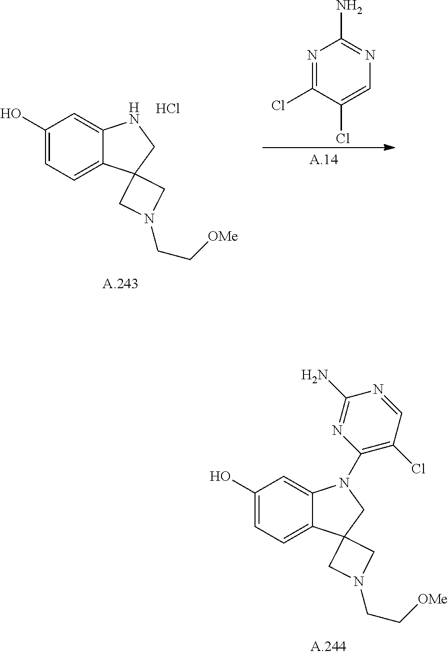

1′-(2-amino-5-chloropyrimidin-4-yl)-1-(2-methoxyethyl)spiro[azetidine-3,3′-indolin]-6′-ol A.244 was prepared from 1-(2-methoxyethyl)spiro[azetidine-3,3′-indolin]-6′-ol hydrochloride A.243 using chemistry similar to that described for compound A.239. LCMS-ESI (POS), M/Z, M+1: Found 362.0.

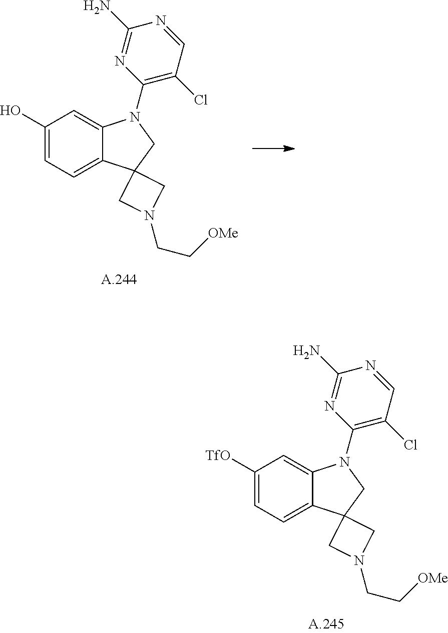

1′-(2-amino-5-chloropyrimidin-4-yl)-1-(2-methoxyethyl)spiro[azetidine-3,3′-indoline]-6′-yl trifluoromethanesulfonate A.245 was prepared from 1′-(2-amino-5-chloropyrimidin-4-yl)-1-(2-methoxyethyl)spiro[azetidine-3,3′-indolin]-6′-ol A.244 using chemistry similar to that described for compound A.240. ¹H NMR (400 MHz, CHLOROFORM-d) δ ppm 8.09 (1H, s), 7.59 (1H, d, J=2.3 Hz), 7.54 (1H, d, J=8.2 Hz), 6.89 (1H, dd, J=8.2, 2.3 Hz), 5.11 (2H, br. s.), 4.59 (2H, s), 3.48-3.55 (2H, m), 3.38-3.47 (4H, m), 3.34 (3H, s), 2.71 (2H, t, J=5.3 Hz). LCMS-ESI (POS), M/Z, M+1: Found 494.0.

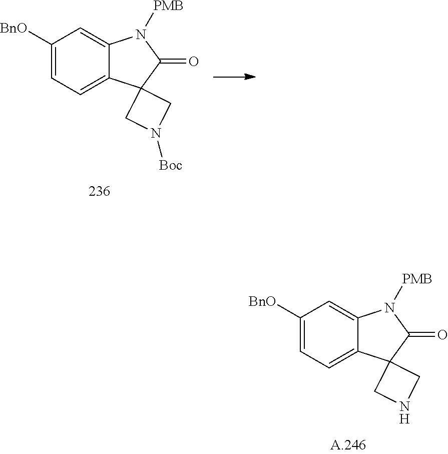

### 6′-(benzyloxy)-1′-(4-methoxybenzyl)spiro[azetidine-3,3′-indolin]-2′-one A.246

A stirred solution of tert-butyl 6′-(benzyloxy)-1′-(4-methoxybenzyl)-2′-oxo-1H-spiro[azetidine-3,3′-indoline]-1-carboxylate A.236 (0.83 g, 0.16 mmol) in dichloromethane (10 mL) was treated with TFA (2.0 mL) at room temperature for 2 h. Upon workup, the crude mixture was poured into ice and 2 N NaOH aqueous solution and extracted with ethyl acetate (2×). The combined organics were dried over Na₂SO₄ and concentrated in vacuo to give 6′-(Benzyloxy)-1′-(4-methoxybenzyl)spiro[azetidine-3,3′-indol]-2′(1′H)-one A.246 which was directly taken onto the next step. LCMS-ESI (POS), M/Z, M+1: Found 401.0.

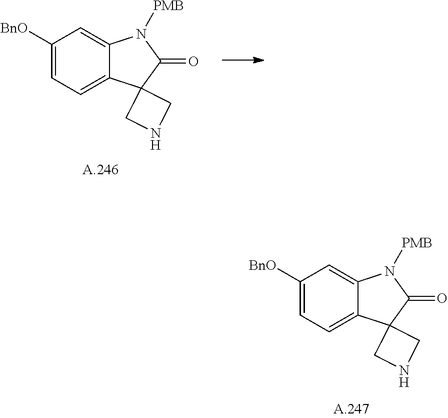

6′-(benzyloxy)-1′-(4-methoxybenzyl)spiro[azetidine-3,3′-indoline] A.247 was prepared from 6′-(benzyloxy)-1′-(4-methoxybenzyl)spiro[azetidine-3,3′-indolin]-2′-one A.246 using chemistry similar to that described for compound A.237. LCMS-ESI (POS), M/Z, M+1: Found 387.2.

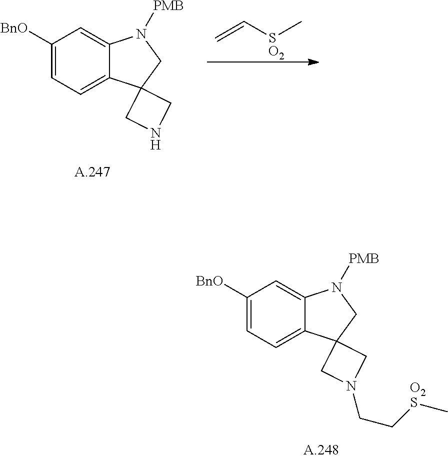

]6′-(benzyloxy)-1′-(4-methoxybenzyl)-1-(2-(methylsulfonyl)ethyl)spiro[azetidine-3,3′-indoline] A.248

A mixture of 6′-(benzyloxy)-1′-(4-methoxybenzyl)spiro[azetidine-3,3′-indoline] A.247 and vinyl methyl sulfone (1.0 eq, 0.076 g) in a mixed solvents of methanol (20 mL) and ethyl acetate (5 mL) was stirred at reflux for 15 h. Upon workup, the volatiles were removed in vacuo to give 6′-(benzyloxy)-1′-(4-methoxybenzyl)-1-(2-(methylsulfonyl)ethyl)spiro[azetidine-3,3′-indoline] A.248 which was directly taken onto the next step. LCMS-ESI (POS), M/Z, M+1: Found 493.1.

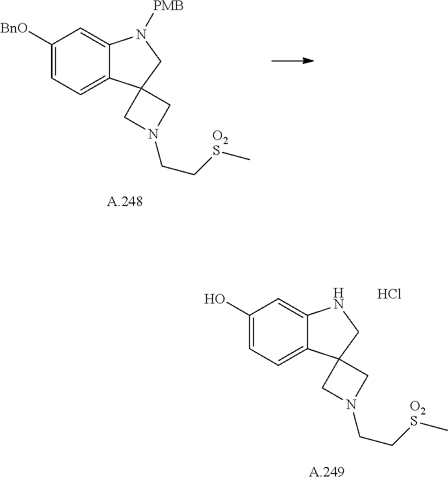

1-(2-(methylsulfonyl)ethyl)spiro[azetidine-3,3′-indolin]-6′-ol hydrochloride A.249 was prepared from 6′-(benzyloxy)-1′-(4-methoxybenzyl)-1-(2-(methylsulfonyl)ethyl)spiro[azetidine-3,3′-indoline] A.248 using chemistry similar to that described for compound A.238. LCMS-ESI (POS), M/Z, M+1: Found 283.0.

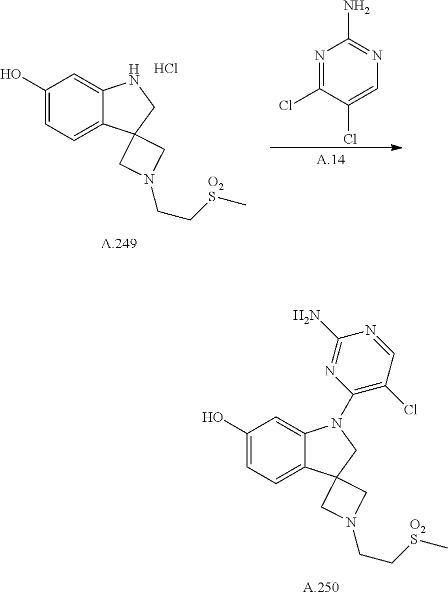

1′-(2-amino-5-chloropyrimidin-4-yl)-1-(2-(methylsulfonyl)ethyl)spiro[azetidine-3,3′-indolin]-6′-ol A.250 was prepared from 1-(2-(methylsulfonyl)ethyl)spiro[azetidine-3,3′-indolin]-6′-ol hydrochloride A.249 and A.14 using chemistry similar to that described for compound A.239. LCMS-ESI (POS), M/Z, M+1: Found 410.0.

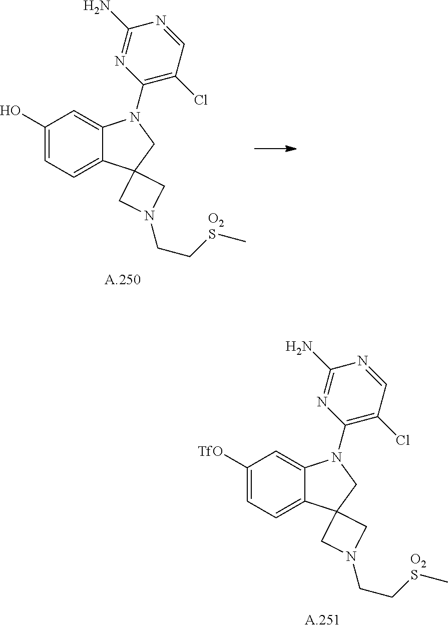

1′-(2-amino-5-chloropyrimidin-4-yl)-1-(2-(methylsulfonyl)ethyl)spiro[azetidine-3,3′-indoline]-6′-yl trifluoromethanesulfonate A.251 was prepared from 1′-(2-amino-5-chloropyrimidin-4-yl)-1-(2-(methylsulfonyl)ethyl)spiro[azetidine-3,3′-indolin]-6′-ol A.250 using chemistry similar to that described for compound A.240. LCMS-ESI (POS), M/Z, M+1: Found 542.0

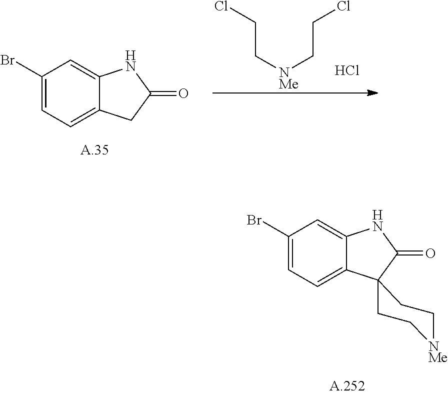

6-Bromo-1′-methylspiro[indole-3,4′-piperidin]-2-one A.252 was prepared by the method of Goehring, R. R., Org. Prep. Proced. Int. 1995, 27(6), 691. A stirred solution of 6-bromoindolin-2-one A.35 (4.4 g, 9.43 mmol) in dry THF (40 mL) was cooled to −78° C. in an acetone-dry ice bath before a solution of NaHMDS in THF (2 M) (52 mL, 5 eq.) was dropwise introduced through a syringe under a nitrogen atmosphere over a period of 25 min. The resulting mixture was stirred at −78° C. for 1 h 15 min before mechlorethamine hydrochloride was added in one portion as a solid. The resulting mixture was stirred at −78° C. for 40 min and at ambient temperature for 3 days. The reaction mixture was cooled in an ice-water bath before quenched with water followed by ice and aqueous 2 N NaOH, and then extracted with ethyl acetate (3×). The combined organics were dried over Na₂SO₄. The residue after concentration in vacuo was subjected to Combi-Flash column chromatography (methanol/triethylamine/dichloromethane) to give 6-bromo-1′-methylspiro[indole-3,4′-piperidin]-2-one A.252 (4.0 g, 65%) as an off-white solid. ¹H NMR (400 MHz, DMSO-d₆) δ 10.54 (br s, 1H), 7.42 (d, J=7.8 Hz, 1H), 7.15 (dd, J=7.8, 1.7 Hz, 1H), 7.00 (d, J=1.7 Hz, 1H), 2.95-2.75 (m, 4H), 1.90-1.65 (m, 4H). LCMS-ESI (POS), M/Z, M+1: Found 295.0 and 297.0.

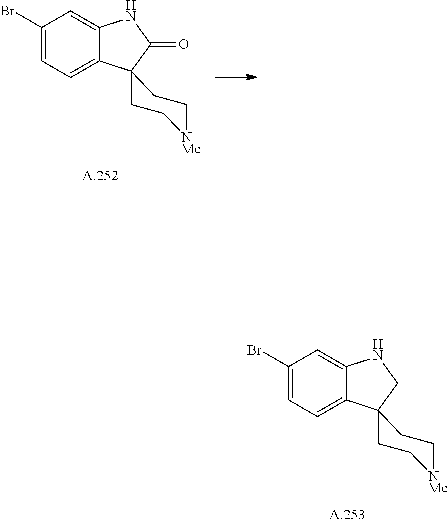

6-bromo-1′-methylspiro[indoline-3,4′-piperidine] A.253 was prepared by the method of Kucerovy, A.; Hathaway, J. S.; Mattner, P. G.; Repic, O. Synth. Commun. 1992, 22, 729. To a stirred solution of 6-bromo-1′-methylspiro[indole-3,4′-piperidin]-2-one A.252 (1.5 g, 5.08 mmol) in dry toluene (80 mL), which was preheated at 80° C. in an oil bath, was dropwise added a solution of Red-AL in toluene (3 M) (5.1 mL, 3 eq.) under a nitrogen atmosphere. The resulting mixture was stirred at this temperature for 2 h. Upon workup the mixture was cooled in an ice-salt bath before quenched with ice-cold aqueous 2 N NaOH. The mixture was further diluted with ice-cold aqueous 2 N NaOH and extracted with ethyl acetate (3×). The combined organics were dried over anhydrous Na₂SO₄ and concentrated in vacuo to give pure 6-bromo-1′-methylspiro[indoline-3,4′-piperidine] A.253 (>90% yield). ¹H NMR (400 MHz, DMSO-d₆) δ 6.91 (d, J=7.8 Hz, 1H), 6.65 (dd, J=7.8, 1.7 Hz, 1H), 6.58 (d, J=1.7 Hz, 1H), 5.81 (br s, 1H), 3.38 (s, 2H), 2.69 (br d, J=11.6 Hz, 2H), 2.19 (s, 3H), 1.97 (t, J=11.6 Hz, 2H), 1.75 (td, J=12.7, 3.6 Hz, 2H), 1.55 (d, J=12.7 Hz, 2H). LCMS-ESI (POS), M/Z, M+1: Found 281.0 and 283.0.

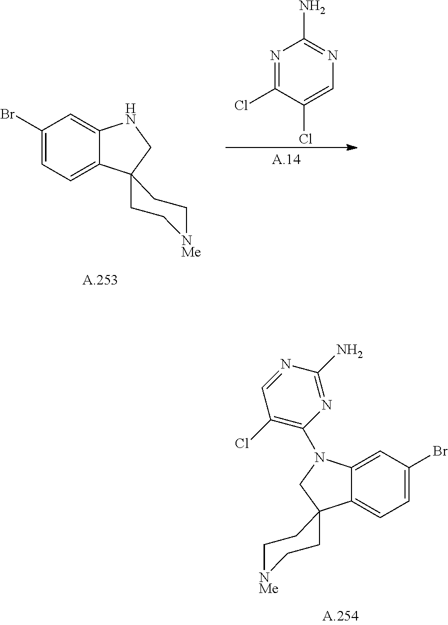

4-(6-bromo-1′-methylspiro[indoline-3,4′-piperidine]-1-yl)-5-chloropyrimidin-2-amine A.254 was prepared from 6-bromo-1′-methylspiro[indoline-3,4′-piperidine] A.253 using chemistry similar to that described for compound A.239. ¹H NMR (400 MHz, DMSO-d₆) 8 ppm 8.12 (1H, s), 7.37 (1H, s), 7.18 (1H, d, J=8.0 Hz), 7.08 (1H, d, J=8.0 Hz), 6.71 (2H, s), 4.03 (2H, s), 2.73 (2H, d, J=11.7 Hz), 2.19 (3H, s), 1.91-2.03 (2H, m), 1.84 (2H, td, J=12.4, 3.3 Hz), 1.59 (2H, d, J=12.8 Hz). LCMS-ESI (POS), M/Z, M+1: Found 408.1 and 410.0.

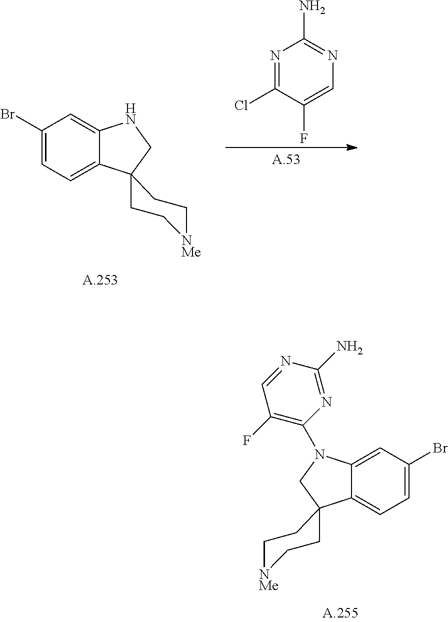

44-(6-bromo-1′-methylspiro[indoline-3,4′-piperidine]-1-yl)-5-fluoropyrimidin-2-amine A.255 was prepared from 6-bromo-1′-methylspiro[indoline-3,4′-piperidine] A.253 and 4-chloro-5-fluoro-2-pyrimidinamine A.53 using chemistry similar to that described for compound A.239. (an off-white solid). ¹H NMR (500 MHz, DMSO-d₆) δ ppm 8.14 (1H, s), 8.03 (1H, d, J=5.9 Hz), 7.17-7.23 (1H, m), 7.09-7.15 (1H, m), 6.43 (2H, s), 4.06 (2H, d, J=4.2 Hz), 2.74 (2H, d, J=11.5 Hz), 2.20 (3H, s), 1.95 (2H, t, J=11.5 Hz), 1.79-1.90 (2H, m), 1.61 (2H, d, J=13.0 Hz). LCMS-ESI (POS), M/Z, M+1: Found 392.0 and 394.0.

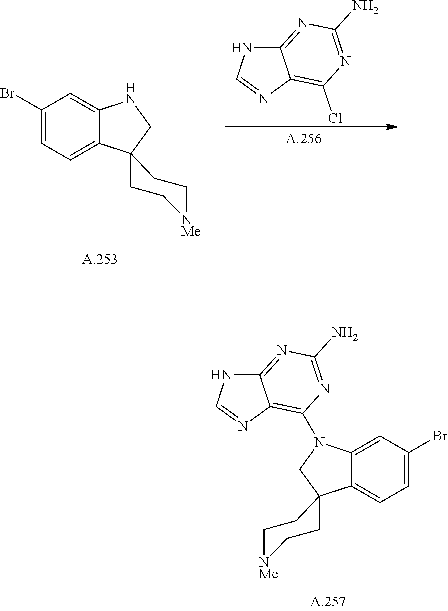

6-(6-bromo-F-methylspiro[indoline-3,4′-piperidine]-1-yl)-9H-purin-2-amine A.257 was prepared from 6-bromo-1′-methylspiro[indoline-3,4′-piperidine] A.253 and 6-chloro-9H-purin-2-amine A.256 using chemistry similar to that described for compound A.239. (an off-white solid). ¹H NMR (500 MHz, DMSO-d₆) δ ppm 8.77 (1H, d, J=1.7 Hz), 7.87 (1H, s), 7.16-7.22 (1H, m), 7.09-7.14 (1H, m), 6.14 (2H, s), 4.60 (2H, s), 2.78 (2H, d, J=11.5 Hz), 2.23 (3H, s), 1.95-2.05 (2H, m), 1.87 (2H, td, J=12.8, 3.5 Hz), 1.62 (2H, d, J=12.7 Hz). LCMS-ESI (POS), M/Z, M+1: Found 414.1 and 416.1.

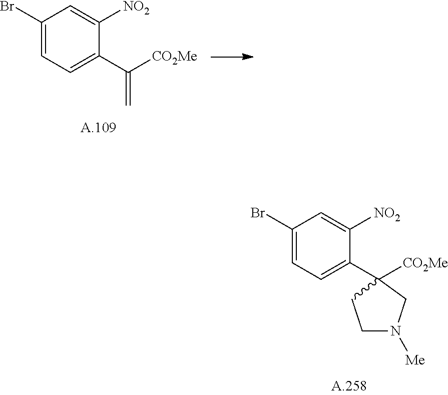

(rac)-Methyl 3-(4-bromo-2-nitrophenyl)-1-methylpyrrolidine-3-carboxylate was prepared by the method of Cravotto, G.; Giovenzana, G. B.; Pilati, T.; Sisti, M.; Palmisano, G. J. Org. Chem. 2001, 66, 8447-8453.

To a stirred solution of methyl 2-(4-bromo-2-nitrophenyl)acrylate A.109 (5.5 g, 19.22 mmol) in toluene (180 mL) was added paraformaldehyde (7.34 g, 12 eq.) in one portion at rt. The 500 mL round-bottomed flask was fitted with a Dean-Stark reflux condenser filled with 3 Å molecular sieves and the resulting mixture was heated to reflux (˜140° C.). under nitrogen. Sarcosine (5.24 g, 3 eq.) was added portionwise to the reaction mixture during a period of 1 h. Then the mixture was refluxed for a further 5.5 h. After cooling, the mixture was vacuum filtered and concentrated in vacuo and the residue was purified on a column of silica gel (methanol/dichloromethane) to give methyl 3-(4-bromo-2-nitrophenyl)-1-methylpyrrolidine-3-carboxylate A.258 (4.7 g, 72% yield) as a light yellow solid. ¹H NMR (500 MHz, DMSO-d₆) δ ppm 8.13 (1H, br. s.), 7.94 (1H, d, J=8.3 Hz), 7.79 (1H, d, J=8.8 Hz), 3.54 (3H, s), 3.24 (1 H, d, J=9.8 Hz), 2.87 (1H, br. s.), 2.66-2.81 (2H, m), 2.42 (1H, q, J=8.0 Hz), 2.29 (3H, s), 1.95-2.06 (1H, m). LCMS-ESI (POS), M/Z, M+1: Found 343.3 and 345.3.

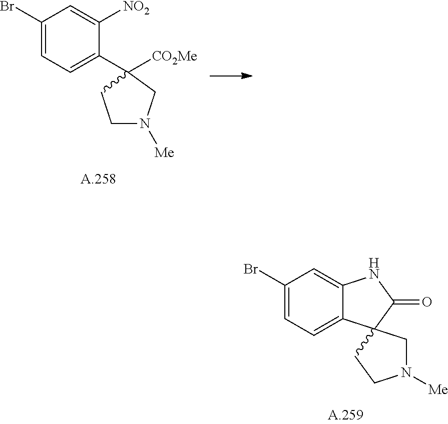

### (rac)-6-bromo-1′-methylspiro[indoline-3,3′-pyrrolidin]-2-one A.259

Ref. Quallich, G. J.; Morrissey, P. M. Synthesis 1993, 51-53.

To a stirred solution of methyl 3-(4-bromo-2-nitrophenyl)-1-methylpyrrolidine-3-carboxylate (4.7 g, 13.70 mmol) in glacial acetic acid (100 mL) was added iron powder (3.82 g, 5 eq.) and the resulting mixture was heated in an oil bath at 100° C. for 4 h. After cooling the mixture was vacuum filtered through a layer of celite. The filtrate was concentrated under high vacuum and the residue was purified on a column of silica gel (methanol/dichloromethane with triethylamine) to give 6-bromo-1′-methylspiro[indoline-3,3′-pyrrolidin]-2-one A.259 in nearly quantitative yield as an off-white solid. ¹H NMR (400 MHz, DMSO-d₆) δ ppm 10.47 (1H, br. s.), 7.25 (1H, d, J=8.1 Hz), 7.14 (1H, d), 6.95 (1H, d, J=1.8 Hz), 2.97-3.08 (1H, m), 2.73 (1 H, d, J=8.8 Hz), 2.56 (1H, d, J=8.8 Hz), 2.43-2.49 (1H, m), 2.33 (3H, s), 2.12-2.22 (1H, m), 1.84-1.94 (1H, m). LCMS-ESI (POS), M/Z, M+1: Found 281.4 and 283.3.

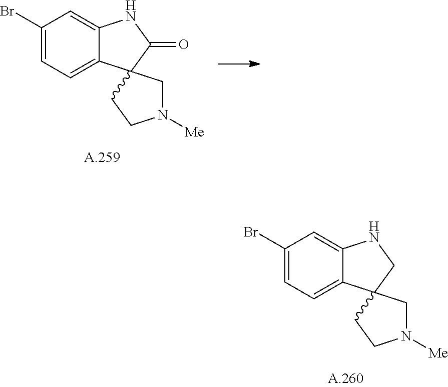

### (rac)-6-bromo-1′-methylspiro[indoline-3,3′-pyrrolidine] A.260

Ref. Kucerovy, A.; Hathaway, J. S.; Mattner, P. G.; Repic, O. Synth. Commun. 1992, 22, 729. To a stirred solution of 6-bromo-1′-methylspiro[indoline-3,3′-pyrrolidin]-2-one A.259 (3.5 g, 12.45 mmol) in dry toluene (40 mL) which was preheated at 85° C. in an oil bath was added dropwise a solution of Red-AL in toluene (3 M) (11 mL, 3 eq.) under nitrogen. The resulting mixture was heated at this temperature for 2 h and then cooled in an ice-salt bath followed by quenching with 2 N NaOH (aq.). After further dilution with 2 N NaOH (aq.), the mixture was extracted with ethyl acetate (3×) and the combined extracts were dried over Na₂SO₄ and concentrated in vacuo to give pure 6-bromo-1′-methylspiro[indoline-3,3′-pyrrolidine] A.260 (>90% yield) as an off-white solid. ¹H NMR (400 MHz, DMSO-d₆) δ ppm 6.97 (1H, d, J=7.4 Hz), 6.67 (1H, dd, J=7.8, 1.6 Hz), 6.59 (1H, d, J=2.0 Hz), 5.75 (1H, s), 3.39-3.43 (1H, m), 3.31-3.34 (1H, m), 2.61-2.69 (1H, m), 2.59 (1H, d, J=9.0 Hz), 2.51-2.56 (1H, m), 2.37 (1 H, d, J=9.0 Hz), 2.26 (3H, s), 1.83-2.01 (2H, m). LCMS-ESI (POS), M/Z, M+1: Found 267.4 and 269.4.

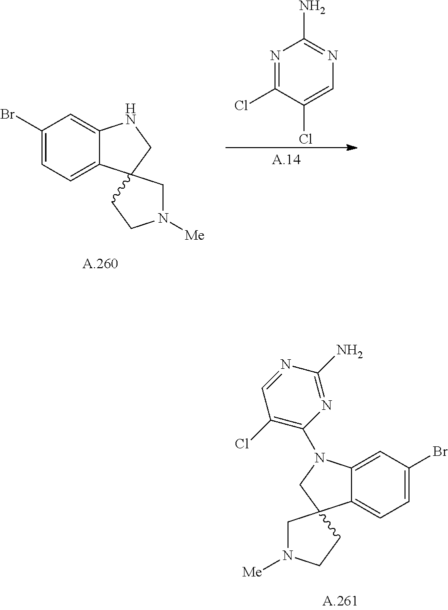

4-(6-bromo-1′-methylspiro[indoline-3,3′-pyrrolidine]-1-yl)-5-chloropyrimidin-2-amine A.261 was prepared from 6-bromo-1′-methylspiro[indoline-3,3′-pyrrolidine] A.260 using chemistry similar to that described for compound A.239. ¹H NMR (500 MHz, DMSO-d₆) δ ppm 8.10 (1H, s), 7.45 (1H, d, J=2.0 Hz), 7.23 (1H, d, J=8.3 Hz), 7.11 (1H, dd, J=8.1, 1.7 Hz), 6.69 (2H, s), 4.16 (1H, d), 4.08 (1H, d), 2.70-2.77 (1H, m), 2.67 (1H, d, J=9.3 Hz), 2.52-2.57 (1H, m), 2.41 (1H, d, J=8.8 Hz), 2.27 (3H, s), 1.95-2.12 (2H, m). LCMS-ESI (POS), M/Z, M+1: Found 394.0 and 396.0.

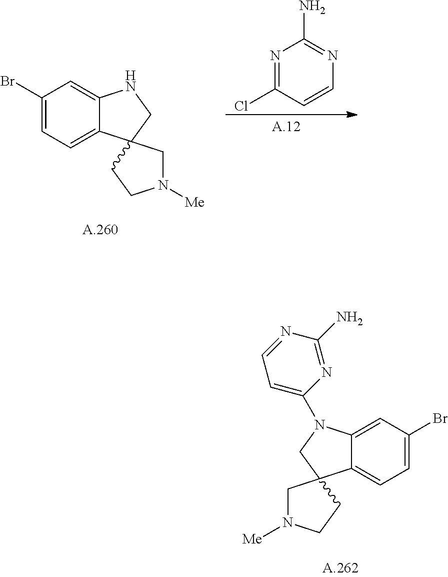

4-(6-bromo-1′-methylspiro[indoline-3,3′-pyrrolidine]-1-yl)pyrimidin-2-amine A.262 was prepared from 6-bromo-1′-methylspiro[indoline-3,4′-piperidine] A.260 and 4-chloro-2-pyrimidinamine A.12 using chemistry similar to that described for compound A.239. (an off-white solid). ¹H NMR (500 MHz, DMSO-d₆) δ ppm 8.60 (1H, d, J=2.0 Hz), 7.97 (1H, d, J=5.9 Hz), 7.23 (1H, d, J=7.8 Hz), 7.10 (1H, dd, J=7.8, 2.0 Hz), 6.43 (2H, br. s.), 6.04 (1H, d, J=5.9 Hz), 3.93-3.99 (1H, m), 3.85-3.91 (1H, m), 2.76 (1H, d, J=5.9 Hz), 2.68 (1H, d, J=8.8 Hz), 2.55-2.63 (1H, m), 2.48 (1H, d), 2.29 (3H, s), 2.03-2.13 (2H, m). LCMS-ESI (POS), M/Z, M+1: Found 360.0 and 362.0.

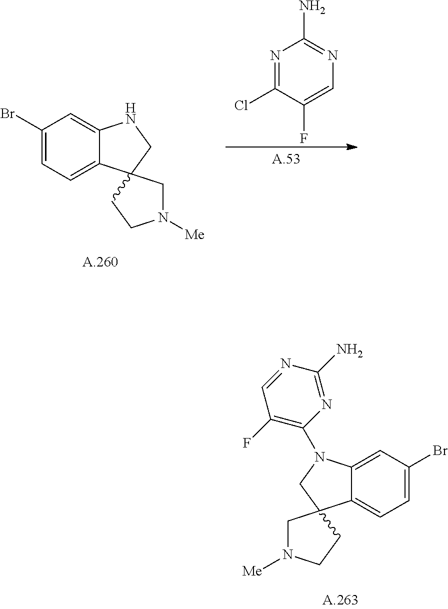

4-(6-bromo-1′-methylspiro[indoline-3,3′-pyrrolidine]-1-yl)-5-fluoropyrimidin-2-amine A.263 was prepared from 6-bromo-1′-methylspiro[indoline-3,4′-piperidine] A.260 and 4-chloro-5-fluoro-2-pyrimidinamine A.53 using chemistry similar to that described for compound A.239. (an off-white solid) ¹H NMR (400 MHz, DMSO-d₆) δ ppm 8.19 (1H, d, J=1.8 Hz), 8.02 (1H, d, J=5.9 Hz), 7.25 (1H, d), 7.15 (1H, dd, J=8.1, 1.8 Hz), 6.41 (2H, s), 4.17-4.25 (1H, m), 4.07-4.14 (1H, m), 2.68-2.80 (2H, m), 2.52-2.59 (1H, m), 2.43 (1H, d, J=9.2 Hz), 2.28 (3H, s), 2.07 (2H, t, J=7.1 Hz). LCMS-ESI (POS), M/Z, M+1: Found 378.0 and 380.0.

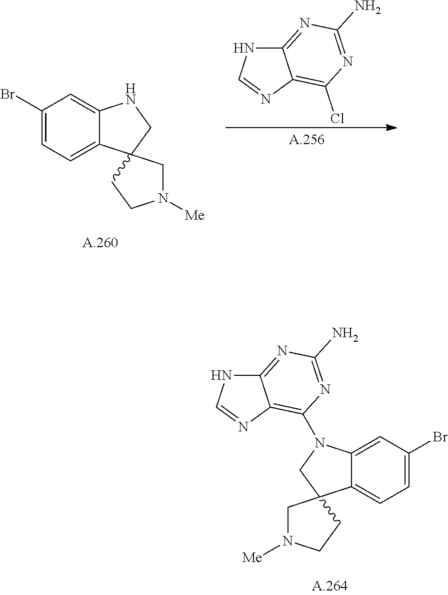

6-(6-bromo-1′-methylspiro[indoline-3,3′-pyrrolidine]-1-yl)-9H-purin-2-amine A.264 was prepared from 6-bromo-1′-methylspiro[indoline-3,4′-piperidine] A.260 and 6-chloro-9H-purin-2-amine A.256 using chemistry similar to that described for compound A.239. (an off-white solid) ¹H NMR (400 MHz, DMSO-d₆) δ ppm 12.37 (1H, br. s.), 8.76 (1H, d, J=1.8 Hz), 7.81 (1H, s), 7.26 (1H, d, J=7.7 Hz), 7.14 (1H, dd, J=8.1, 1.8 Hz), 6.13 (2H, s), 4.81 (1H, d, J=11.7 Hz), 4.53 (1H, d, J=12.1 Hz), 2.76-2.86 (1H, m), 2.72 (1H, d, J=9.1 Hz), 2.54-2.62 (1H, m), 2.46 (2H, d, J=8.8 Hz), 2.30 (3H, s), 2.01-2.18 (2H, m). LCMS-ESI (POS), M/Z, M+1: Found 400.1 and 402.1.

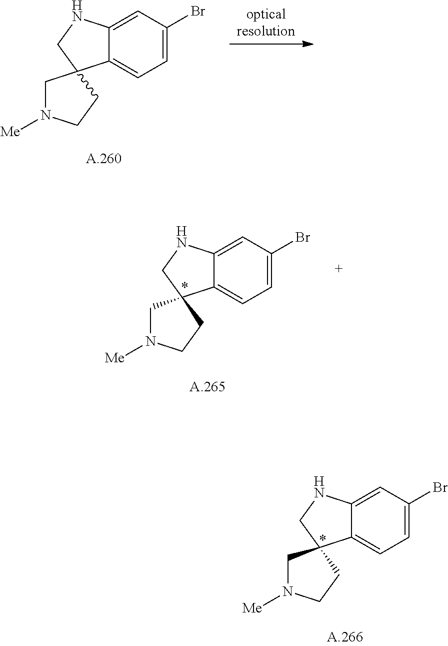

(3S*)-6-bromo-1′-methylspiro[indoline-3,3′-pyrrolidine] A.265 and (3R*)-6-bromo-1′-methylspiro[indoline-3,3′-pyrrolidine] A.266 (stereochemistry arbitrarily assigned) were obtained from optical resolution of (rac)-6-bromo-1′-methylspiro[indoline-3,3′-pyrrolidine] A.260 by the following method. Instrument: Agilent 1100 series. Column: OD-preparative (50 mm×500 mm). Solvents: 1% isopropanol in hexanes. Gradient: isochratic. Amount of sample per injection: 250 mg in methanol (6 mL). Separation quality: baseline separation. The first peak eluting off of the OD column provided (3S*)-6-bromo-1′-methylspiro[indoline-3,3′-pyrrolidine] A.265 in 94% ee. ¹H NMR (400 MHz, DMSO-d₆) δ ppm 6.97 (1H, d, J=7.4 Hz), 6.67 (1H, dd, J=7.8, 1.6 Hz), 6.59 (1H, d, J=2.0 Hz), 5.75 (1H, s), 3.39-3.43 (1H, m), 3.31-3.34 (1H, m), 2.61-2.69 (1H, m), 2.59 (1H, d, J=9.0 Hz), 2.51-2.56 (1H, m), 2.37 (1H, d, J=9.0 Hz), 2.26 (3H, s), 1.83-2.01 (2H, m). LCMS-ESI (POS), M/Z, M+1: Found 267.4 and 269.4.

The second peak furnished (3R*)-6-bromo-1′-methylspiro[indoline-3,3′-pyrrolidine] A.266 in 99% ee.

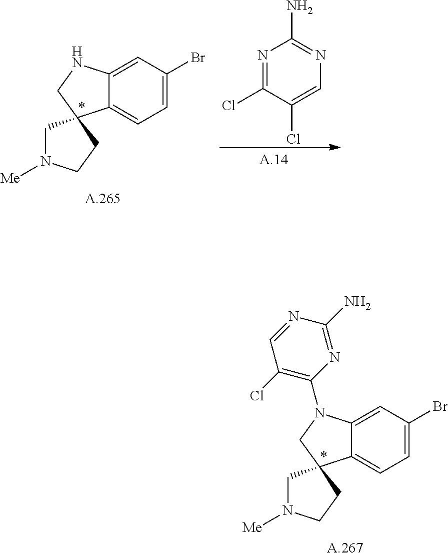

4-((3R*)-6-Bromo-1′-methylspiro[indole-3,3′-pyrrolidin]-1(2H)-yl)-5-chloro-2-pyrimidinamine A.267 was prepared from (3S*)-6-bromo-1′-methylspiro[indoline-3,3′-pyrrolidine] A.265 using chemistry similar to that described for compound A.239. ¹H NMR (400 MHz, DMSO-d₆) δ ppm 8.10 (1H, s), 7.45 (1H, d, J=1.6 Hz), 7.23 (1H, d, J=7.8 Hz), 7.10 (1H, dd, J=7.8, 1.6 Hz), 6.68 (2H, s), 4.16 (1H, d), 4.08 (1H, d), 2.69-2.78 (1H, m), 2.67 (1H, d, J=9.0 Hz), 2.52-2.58 (1H, m), 2.42 (1H, d, J=9.0 Hz), 2.27 (3H, s), 1.97-2.12 (2H, m). LCMS-ESI (POS), M/Z, M+1: Found 394.0 and 396.0.

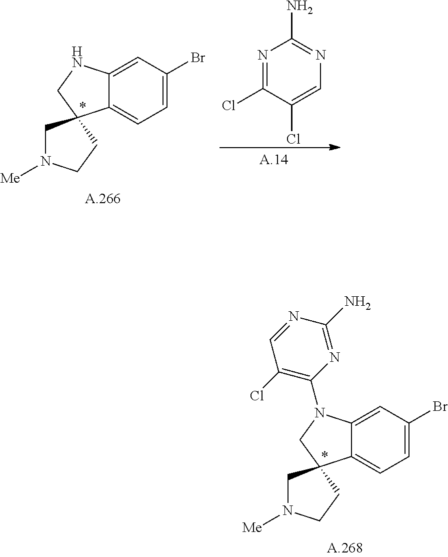

4-((3R*)-6-Bromo-1′-methylspiro[indole-3,3′-pyrrolidin]-1(2H)-yl)-5-chloro-2-pyrimidinamine A.268 was prepared from (3R*)-6-bromo-1′-methylspiro[indoline-3,3′-pyrrolidine] A.266 using chemistry similar to that described for compound A.239.

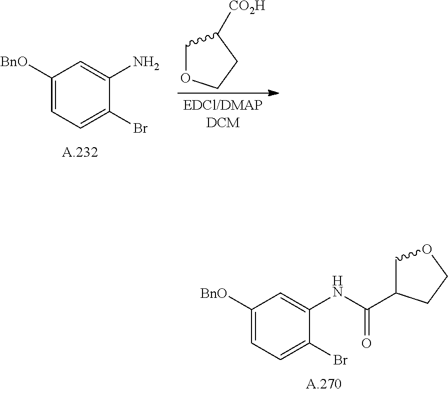

N-(5-(benzyloxy)-2-bromophenyl)tetrahydrofuran-3-carboxamide A.270 was prepared from 5-(benzyloxy)-2-bromoaniline A.232 and tetrahydro-3-furancarboxylic acid using chemistry similar to that described for compound A.234. (a white solid) ¹H NMR (400 MHz, DMSO-d₆) δ ppm 9.46 (1H, s), 7.53 (1H, d, J=8.6 Hz), 7.28-7.47 (6H, m), 6.84 (1H, dd, J=8.8, 2.9 Hz), 5.09 (2H, s), 3.92 (1H, t, J=8.0 Hz), 3.77 (2H, q, J=6.8 Hz), 3.70 (1H, q, J=7.0 Hz), 3.22-3.28 (1H, m), 2.09 (2H, q, J=6.8 Hz). LCMS-ESI (POS), M/Z, M+1: Found 376.1 and 378.1.

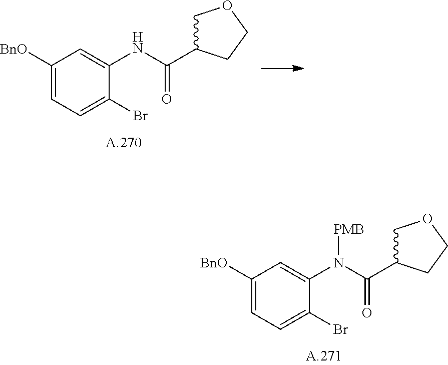

N-(5-(benzyloxy)-2-bromophenyl)-N-(4-methoxybenzyl)tetrahydrofuran-3-carboxamide A.271 (a colorless viscous liquid), was prepared from N-(5-(benzyloxy)-2-bromophenyl)tetrahydrofuran-3-carboxamide A.270 using chemistry similar to that described for compound A.235. ¹H NMR (400 MHz, DMSO-d₆) δ ppm 7.64 (1H, d, J=9.0 Hz), 7.29-7.42 (6H, m), 7.06 (2H, dd, J=8.6, 2.7 Hz), 7.00 (1H, dt, J=9.0, 3.5 Hz), 6.84 (2H, dd, J=8.6, 2.7 Hz), 6.61 (1H, t, J=3.1 Hz), 5.20 (1H, dd, J=14.3, 3.3 Hz), 4.98 (2H, s), 4.14 (1H, d, J=14.1 Hz), 3.65-3.80 (6H, m), 3.44-3.61 (2H, m), 2.59-2.72 (1H, m), 2.04-2.17 (1H, m). LCMS-ESI (POS), M/Z, M+1: Found 496.1 and 498.1.

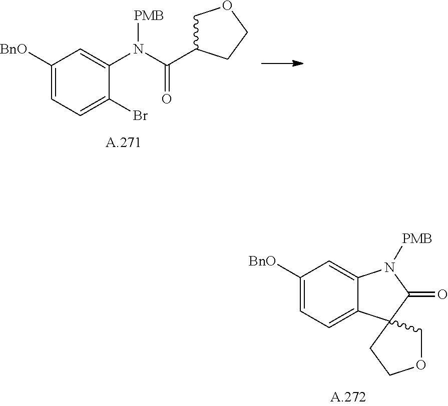

(rac)-6′6′-(benzyloxy)-1′-(4-methoxybenzyl)-4,5-dihydro-2H-spiro[furan-3,3′-indolin]-2′-one A.272 (a colorless viscous liquid) was prepared from N-(5-(benzyloxy)-2-bromophenyl)-N-(4-methoxybenzyl)tetrahydrofuran-3-carboxamide A.271 using chemistry similar to that described for compound A.236. ¹H NMR (400 MHz, DMSO-d₆) δ ppm 7.29-7.46 (6H, m), 7.18-7.26 (3H, m), 6.87 (2H, d, J=8.6 Hz), 6.62-6.68 (2H, m), 5.06 (2H, s), 4.81 (2H, s), 4.05-4.15 (2H, m), 3.85-3.90 (1H, m), 3.77 (1H, d, J=8.2 Hz), 3.71 (3H, s), 2.32 (1H, ddd, J=12.3, 7.4, 7.2 Hz), 2.08 (1H, dt, J=12.6, 6.4 Hz). LCMS-ESI (POS), M/Z, M+1: Found 416.2.

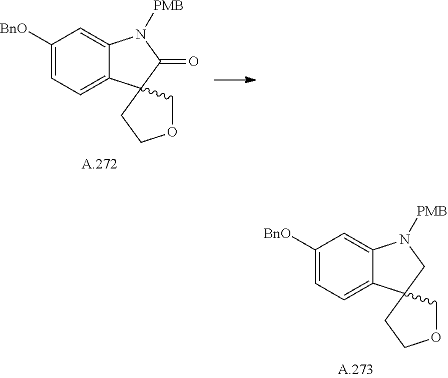

(rac)-6′-(benzyloxy)-1′-(4-methoxybenzyl)-4,5-dihydro-2H-spiro[furan-3,3′-indoline] A.273 (a colorless viscous liquid) was prepared from 6′-(benzyloxy)-1′-(4-methoxybenzyl)-4,5-dihydro-2H-spiro[furan-3,3′-indolin]-2′-one A.272 using chemistry similar to that described for compound A.237. LCMS-ESI (POS), M/Z, M+1: Found 402.2.

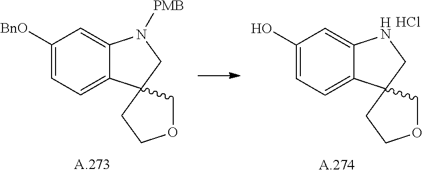

(rac)-1′,2′,4,5-Tetrahydrospiro[furan-3,3′-indol]-6′-ol hydrochloride A.274 (a colorless viscous liquid) was prepared from 6′-(benzyloxy)-1′-(4-methoxybenzyl)-4,5-dihydro-2H-spiro[furan-3,3′-indoline] A.273 using chemistry similar to that described for compound A.238. LCMS-ESI (POS), M/Z, M+1: Found 192.1.

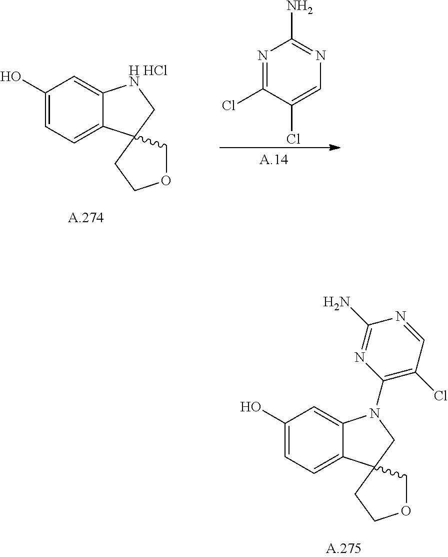

(rac)-1′-(2-Amino-5-chloro-4-pyrimidinyl)-1′,2′,4,5-tetrahydrospiro[furan-3,3′-indol]-6′-ol A.275 (an off-white solid) was prepared from 1′,2′,4,5-tetrahydrospiro[furan-3,3′-indol]-6′-ol hydrochloride A.274 and 4,5-dichloro-2-pyrimidinamine A.14 using chemistry similar to that described for compound A.239. ¹H NMR (400 MHz, DMSO-d₆) δ ppm 8.24 (1H, s), 7.19 (1 H, br. s.), 7.12 (1H, d, J=8.2 Hz), 6.52 (1H, dd, J=7.8, 2.0 Hz), 4.18-4.38 (2H, m), 3.91-4.02 (1H, m), 3.87 (1H, q, J=7.8 Hz), 3.78 (1H, d, J=8.2 Hz), 3.54 (1H, d, J=8.6 Hz), 2.05-2.23 (2H, m). LCMS-ESI (POS), M/Z, M+1: Found 319.1.

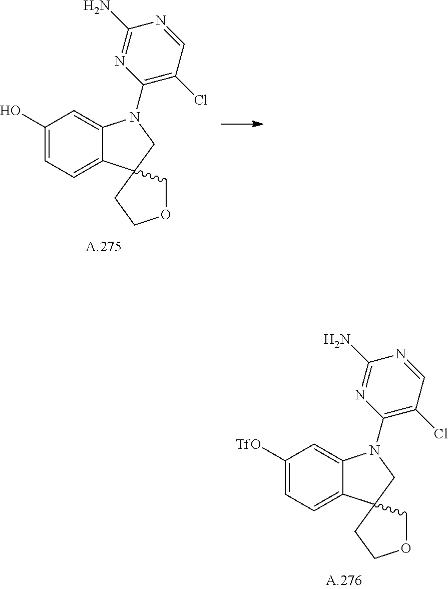

(rac)-1′-(2-amino-5-chloropyrimidin-4-yl)-4,5-dihydro-2H-spiro[furan-3,3′-indoline]-6′-yl trifluoromethanesulfonate A.276 (a colorless solid) was prepared from 1′-(2-amino-5-chloro-4-pyrimidinyl)-1′,2′,4,5-tetrahydrospiro[furan-3,3′-indol]-6′-ol A.275 using chemistry similar to that described for compound A.240. ¹H NMR (400 MHz, CHLOROFORM-d) δ ppm 8.12 (1H, s), 7.52 (1H, d, J=2.3 Hz), 7.23 (1H, d, J=8.6 Hz), 6.88 (1H, dd, J=8.2, 2.3 Hz), 5.07 (2H, br. s.), 4.38 (1H, d), 4.27 (1H, d), 4.07-4.15 (1H, m), 3.99-4.07 (1H, m), 3.88 (1 H, d), 3.79 (1H, d), 2.26-2.36 (1H, m), 2.14-2.22 (1H, m). LCMS-ESI (POS), M/Z, M+1: Found 451.0.

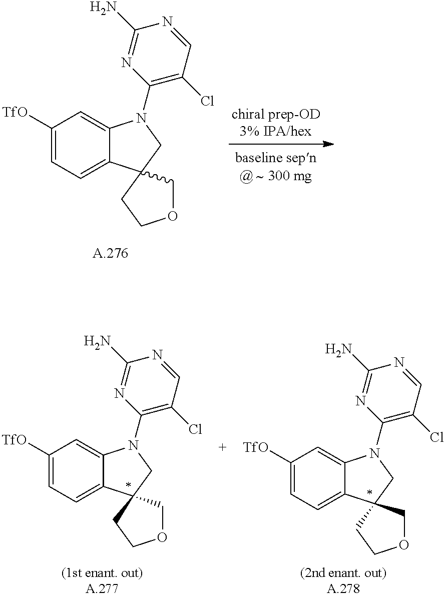

(3R*)-1′-(2-amino-5-chloropyrimidin-4-yl)-4,5-dihydro-2H-spiro[furan-3,3′-indoline]-6′-yl trifluoromethanesulfonate A.277 and (3S*)-1′-(2-amino-5-chloropyrimidin-4-yl)-4,5-dihydro-2H-spiro[furan-3,3′-indoline]-6′-yl trifluoromethanesulfonate A.278 were obtained from optical resolution of (rac)-1′-(2-amino-5-chloropyrimidin-4-yl)-4,5-dihydro-2H-spiro[furan-3,3′-indoline]-6′-yl trifluoromethanesulfonate by the following method. Instrument: Agilent 1100 series. Column: OD-preparative (50 mm×500 mm). Solvents: 3% isopropanol in hexanes. Gradient: isochratic. Amount of sample per injection: 300 mg in methanol (6 mL). Separation quality: baseline separation. The first peak eluting off of the OD column provided (3R*)-1′-(2-amino-5-chloropyrimidin-4-yl)-4,5-dihydro-2H-spiro[furan-3,3′-indoline]-6′-yl trifluoromethanesulfonate A.277 as an off-white solid.

The second peak furnished (3S*)-1′-(2-amino-5-chloropyrimidin-4-yl)-4,5-dihydro-2H-spiro[furan-3,3′-indoline]-6′-yl trifluoromethanesulfonate A.278 as an off-white solid. Stereochemistry arbitrarily assigned.

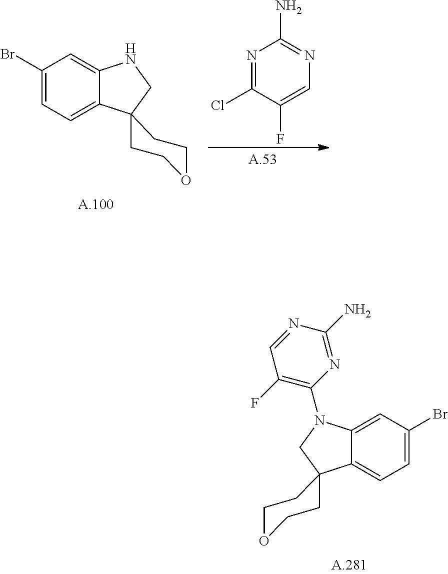

4-(6-Bromo-2′,3′,5′,6′-tetrahydrospiro[indole-3,4′-pyran]-1(2H)-yl)-5-fluoro-2-pyrimidinamine A.281 (an off-white solid) was prepared from 6-bromo-1,2,2′,3′,5′,6′-hexahydrospiro[indole-3,4′-pyran] (A.100) and 4-chloro-5-fluoro-2-pyrimidinamine A.53 using chemistry similar to that described for compound A.239. LCMS-ESI (POS), M/Z, M+1: Found 379.0 and 381.0.

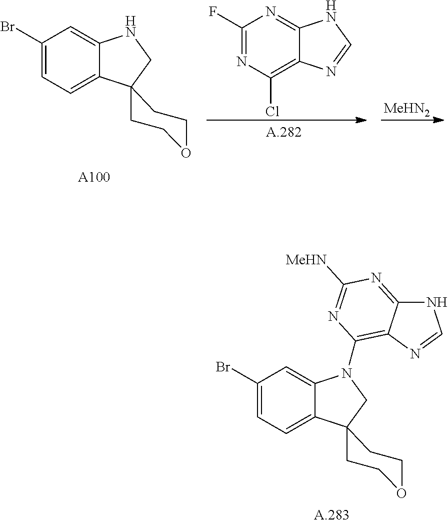

### 6-(6-Bromo-2′,3′,5′,6′-tetrahydrospiro[indole-3,4′-pyran]-1(2H)-yl)-N-methyl-9H-purin-2-amine A.283

To a stirred solution of 6-bromo-1,2,2′,3′,5′,6′-hexahydrospiro[indole-3,4′-pyran] (A.100) (0.24 g, 0.89 mmol) in t-butanol (12 mL) in a pressure vessel was added 6-chloro-2-fluoro-9H-purine A.282 (1.0 eq., 0.18 g) followed by Hunig's base (0.21 mL). The resulting mixture was stirred at 80° C. for 22 h. After cooling, methylamine (2 M solution in THF) was added to this mixture. The resulting mixture was stirred at 85° C. for 15 h. Upon workup, the volatiles were removed in vacuo and the residue was subjected to combi-flash column chromatography (methanol/dichloromethane) to give 6-(6-bromo-2′,3′,5′,6′-tetrahydrospiro[indole-3,4′-pyran]-1(2H)-yl)-N-methyl-9H-purin-2-amine A.283. LCMS-ESI (POS), M/Z, M+1: Found 415.0 and 417.0.

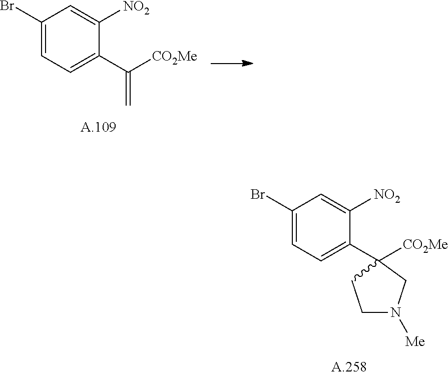

(rac)-Methyl 3-(4-bromo-2-nitrophenyl)-1-methylpyrrolidine-3-carboxylate was prepared by the method of Cravotto, G.; Giovenzana, G. B.; Pilati, T.; Sisti, M.; Palmisano, G. J. Org. Chem. 2001, 66, 8447-8453.

To a stirred solution of methyl 2-(4-bromo-2-nitrophenyl)acrylate A.109 (5.5 g, 19.22 mmol) in toluene (180 mL) was added paraformaldehyde (7.34 g, 12 eq.) in one portion at rt. The 500 mL round-bottomed flask was fitted with a Dean-Stark reflux condenser filled with 3 Å molecular sieves and the resulting mixture was heated to reflux (˜140° C.). under nitrogen. Sarcosine (5.24 g, 3 eq.) was added portionwise to the reaction mixture during a period of 1 h. Then the mixture was refluxed for a further 5.5 h. After cooling, the mixture was vacuum filtered and concentrated in vacuo and the residue was purified on a column of silica gel (methanol/dichloromethane) to give methyl 3-(4-bromo-2-nitrophenyl)-1-methylpyrrolidine-3-carboxylate A.258 (4.7 g, 72% yield) as a light yellow solid. ¹H NMR (500 MHz, DMSO-d₆) δ ppm 8.13 (1H, br. s.), 7.94 (1H, d, J=8.3 Hz), 7.79 (1H, d, J=8.8 Hz), 3.54 (3H, s), 3.24 (1 H, d, J=9.8 Hz), 2.87 (1H, br. s.), 2.66-2.81 (2H, m), 2.42 (1H, q, J=8.0 Hz), 2.29 (3H, s), 1.95-2.06 (1H, m). LCMS-ESI (POS), M/Z, M+1: Found 343.3 and 345.3.

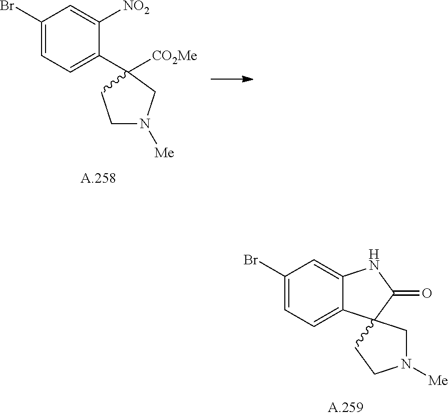

### (rac)-6-bromo-1′-methylspiro[indoline-3,3′-pyrrolidin]-2-one

Ref. Quallich, G. J.; Morrissey, P. M. Synthesis 1993, 51-53.

To a stirred solution of methyl 3-(4-bromo-2-nitrophenyl)-1-methylpyrrolidine-3-carboxylate (4.7 g, 13.70 mmol) in glacial acetic acid (100 mL) was added iron powder (3.82 g, 5 eq.) and the resulting mixture was heated in an oil bath at 100° C. for 4 h. After cooling the mixture was vacuum filtered through a layer of celite. The filtrate was concentrated under high vacuum and the residue was purified on a column of silica gel (methanol/dichloromethane with triethylamine) to give 6-bromo-1′-methylspiro[indoline-3,3′-pyrrolidin]-2-one in nearly quantitative yield as an off-white solid. ¹H NMR (400 MHz, DMSO-d₆) δ ppm 10.47 (1H, br. s.), 7.25 (1H, d, J=8.1 Hz), 7.14 (1H, d), 6.95 (1H, d, J=1.8 Hz), 2.97-3.08 (1H, m), 2.73 (1 H, d, J=8.8 Hz), 2.56 (1H, d, J=8.8 Hz), 2.43-2.49 (1H, m), 2.33 (3H, s), 2.12-2.22 (1H, m), 1.84-1.94 (1H, m). LCMS-ESI (POS), M/Z, M+1: Found 281.4 and 283.3.

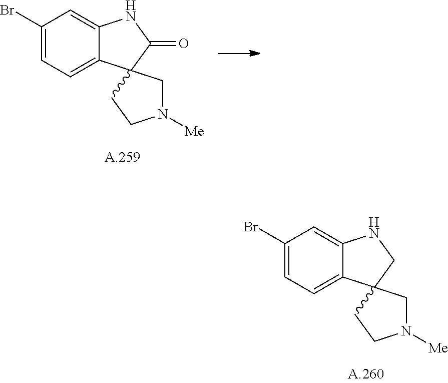

### (rac)-6-bromo-1′-methylspiro[indoline-3,3′-pyrrolidine] A.260

Ref. Kucerovy, A.; Hathaway, J. S.; Mattner, P. G.; Repic, O. Synth. Commun. 1992, 22, 729. To a stirred solution of 6-bromo-1′-methylspiro[indoline-3,3′-pyrrolidin]-2-one A.259 (3.5 g, 12.45 mmol) in dry toluene (40 mL) which was preheated at 85° C. in an oil bath was added dropwise a solution of Red-AL in toluene (3 M) (11 mL, 3 eq.) under nitrogen. The resulting mixture was heated at this temperature for 2 h and then cooled in an ice-salt bath followed by quenching with 2 N NaOH (aq.). After further dilution with 2 N NaOH (aq.), the mixture was extracted with ethyl acetate (3×) and the combined extracts were dried over Na₂SO₄ and concentrated in vacuo to give pure 6-bromo-1′-methylspiro[indoline-3,3′-pyrrolidine] A.260 (>90% yield) as an off-white solid. ¹H NMR (400 MHz, DMSO-d₆) δ ppm 6.97 (1H, d, J=7.4 Hz), 6.67 (1H, dd, J=7.8, 1.6 Hz), 6.59 (1H, d, J=2.0 Hz), 5.75 (1H, s), 3.39-3.43 (1H, m), 3.31-3.34 (1H, m), 2.61-2.69 (1H, m), 2.59 (1H, d, J=9.0 Hz), 2.51-2.56 (1H, m), 2.37 (1 H, d, J=9.0 Hz), 2.26 (3H, s), 1.83-2.01 (2H, m). LCMS-ESI (POS), M/Z, M+1: Found 267.4 and 269.4.

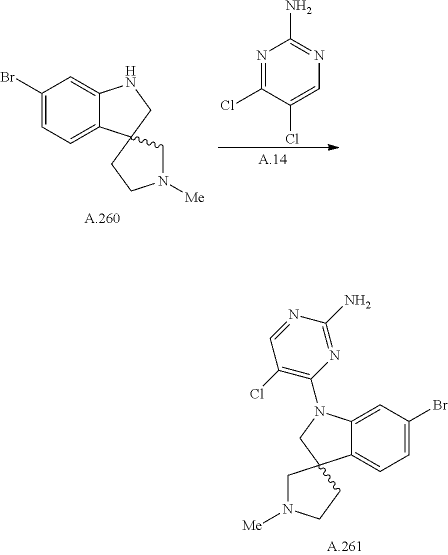

4-(6-bromo-1′-methylspiro[indoline-3,3′-pyrrolidine]-1-yl)-5-chloropyrimidin-2-amine A.261 was prepared from 6-bromo-1′-methylspiro[indoline-3,3′-pyrrolidine] A.260 using chemistry similar to that described for compound A.239. ¹H NMR (500 MHz, DMSO-d₆) δ ppm 8.10 (1H, s), 7.45 (1H, d, J=2.0 Hz), 7.23 (1H, d, J=8.3 Hz), 7.11 (1H, dd, J=8.1, 1.7 Hz), 6.69 (2H, s), 4.16 (1H, d), 4.08 (1H, d), 2.70-2.77 (1H, m), 2.67 (1H, d, J=9.3 Hz), 2.52-2.57 (1H, m), 2.41 (1H, d, J=8.8 Hz), 2.27 (3H, s), 1.95-2.12 (2H, m). LCMS-ESI (POS), M/Z, M+1: Found 394.0 and 396.0.

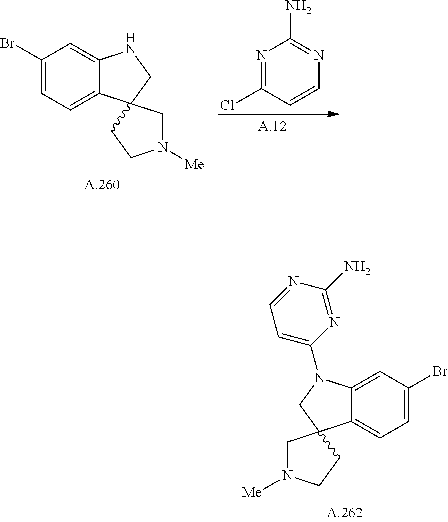

4-(6-bromo-1′-methylspiro[indoline-3,3′-pyrrolidine]-1-yl)pyrimidin-2-amine A.262 was prepared from 6-bromo-1′-methylspiro[indoline-3,4′-piperidine] A.260 and 4-chloro-2-pyrimidinamine A.12 using chemistry similar to that described for compound A.239. (an off-white solid). ¹H NMR (500 MHz, DMSO-d₆) δ ppm 8.60 (1H, d, J=2.0 Hz), 7.97 (1H, d, J=5.9 Hz), 7.23 (1H, d, J=7.8 Hz), 7.10 (1H, dd, J=7.8, 2.0 Hz), 6.43 (2H, br. s.), 6.04 (1H, d, J=5.9 Hz), 3.93-3.99 (1H, m), 3.85-3.91 (1H, m), 2.76 (1H, d, J=5.9 Hz), 2.68 (1H, d, J=8.8 Hz), 2.55-2.63 (1H, m), 2.48 (1H, d), 2.29 (3H, s), 2.03-2.13 (2H, m). LCMS-ESI (POS), M/Z, M+1: Found 360.0 and 362.0.

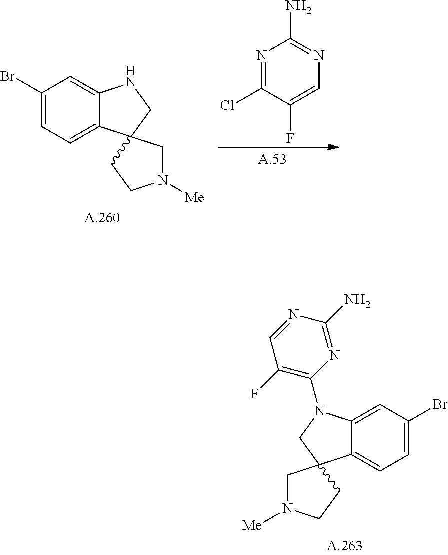

4-(6-bromo-1′-methylspiro[indoline-3,3′-pyrrolidine]-1-yl)-5-fluoropyrimidin-2-amine A.263 was prepared from 6-bromo-1′-methylspiro[indoline-3,4′-piperidine] A.260 and 4-chloro-5-fluoro-2-pyrimidinamine A.53 using chemistry similar to that described for compound A.239. (an off-white solid) ¹H NMR (400 MHz, DMSO-d₆) δ ppm 8.19 (1H, d, J=1.8 Hz), 8.02 (1H, d, J=5.9 Hz), 7.25 (1H, d), 7.15 (1H, dd, J=8.1, 1.8 Hz), 6.41 (2H, s), 4.17-4.25 (1H, m), 4.07-4.14 (1H, m), 2.68-2.80 (2H, m), 2.52-2.59 (1H, m), 2.43 (1H, d, J=9.2 Hz), 2.28 (3H, s), 2.07 (2H, t, J=7.1 Hz). LCMS-ESI (POS), M/Z, M+1: Found 378.0 and 380.0

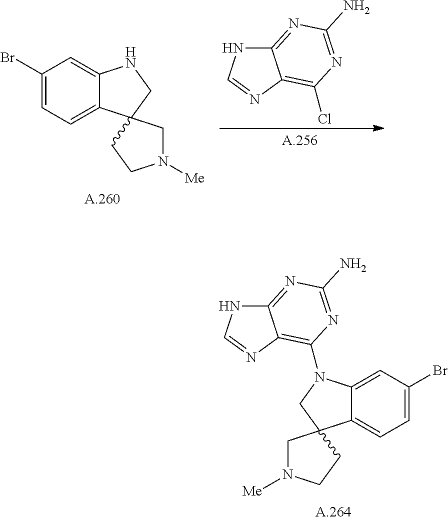

6-(6-bromo-1′-methylspiro[indoline-3,3′-pyrrolidine]-1-yl)-9H-purin-2-amine A.264 was prepared from 6-bromo-1′-methylspiro[indoline-3 ,4′-piperidine] A.260 and 6-chloro-9H-purin-2-amine A.256 using chemistry similar to that described for compound A.239. (an off-white solid) ¹H NMR (400 MHz, DMSO-d₆) δ ppm 12.37 (1H, br. s.), 8.76 (1H, d, J=1.8 Hz), 7.81 (1H, s), 7.26 (1H, d, J=7.7 Hz), 7.14 (1H, dd, J=8.1, 1.8 Hz), 6.13 (2H, s), 4.81 (1H, d, J=11.7 Hz), 4.53 (1H, d, J=12.1 Hz), 2.76-2.86 (1H, m), 2.72 (1H, d, J=9.1 Hz), 2.54-2.62 (1H, m), 2.46 (2H, d, J=8.8 Hz), 2.30 (3H, s), 2.01-2.18 (2H, m). LCMS-ESI (POS), M/Z, M+1: Found 400.1 and 402.1.

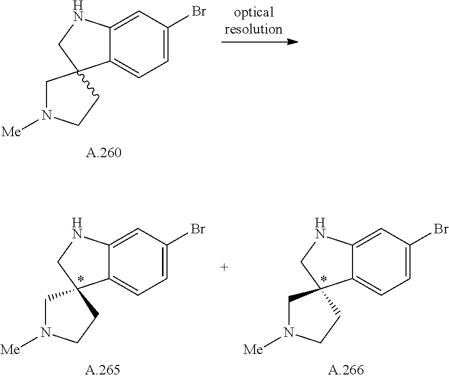

(3S*)-6-bromo-1′-methylspiro[indoline-3,3′-pyrrolidine] A.265 and (3R*)-6-bromo-1′-methylspiro[indoline-3,3′-pyrrolidine] A.266 (stereochemistry arbitrarily assigned) were obtained from optical resolution of (rac)-6-bromo-1′-methylspiro[indoline-3,3′-pyrrolidine] A.260 by the following method. Instrument: Agilent 1100 series. Column: OD-preparative (50 mm×500 mm). Solvents: 1% isopropanol in hexanes. Gradient: isochratic. Amount of sample per injection: 250 mg in methanol (6 mL). Separation quality: baseline separation. The first peak eluting off of the OD column provided (3S*)-6-bromo-1′-methylspiro[indoline-3,3′-pyrrolidine] A.265 in 94% ee. ¹H NMR (400 MHz, DMSO-d₆) δ ppm 6.97 (1H, d, J=7.4 Hz), 6.67 (1H, dd, J=7.8, 1.6 Hz), 6.59 (1H, d, J=2.0 Hz), 5.75 (1H, s), 3.39-3.43 (1H, m), 3.31-3.34 (1H, m), 2.61-2.69 (1H, m), 2.59 (1H, d, J=9.0 Hz), 2.51-2.56 (1H, m), 2.37 (1H, d, J=9.0 Hz), 2.26 (3H, s), 1.83-2.01 (2H, m). LCMS-ESI (POS), M/Z, M+1: Found 267.4 and 269.4.

The second peak furnished (3R*)-6-bromo-1′-methylspiro[indoline-3,3′-pyrrolidine] A.266 in 99% ee. (stereochemistry arbitrarily assigned.)

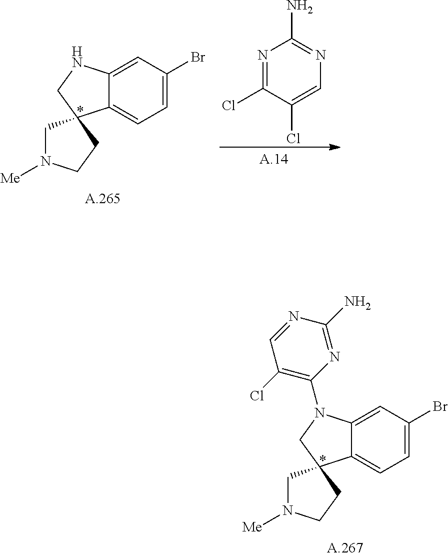

4-((3R*)-6-Bromo-1′-methylspiro[indole-3,3′-pyrrolidin]-1(2H)-yl)-5-chloro-2-pyrimidinamine A.267 was prepared from (3S*)-6-bromo-1′-methylspiro[indoline-3,3′-pyrrolidine] A.265 using chemistry similar to that described for compound A.239. ¹H NMR (400 MHz, DMSO-d₆) δ ppm 8.10 (1H, s), 7.45 (1H, d, J=1.6 Hz), 7.23 (1H, d, J=7.8 Hz), 7.10 (1H, dd, J=7.8, 1.6 Hz), 6.68 (2H, s), 4.16 (1H, d), 4.08 (1H, d), 2.69-2.78 (1H, m), 2.67 (1H, d, J=9.0 Hz), 2.52-2.58 (1H, m), 2.42 (1H, d, J=9.0 Hz), 2.27 (3H, s), 1.97-2.12 (2H, m). LCMS-ESI (POS), M/Z, M+1: Found 394.0 and 396.0.

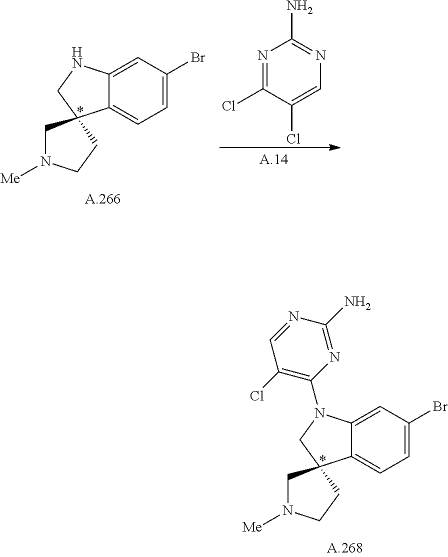

4-((3R*)-6-Bromo-1′-methylspiro[indole-3,3′-pyrrolidin]-1(2H)-yl)-5-chloro-2-pyrimidinamine A.268 was prepared from (3R*)-6-bromo-1′-methylspiro[indoline-3,3′-pyrrolidine] A.266 using chemistry similar to that described for compound A.239.

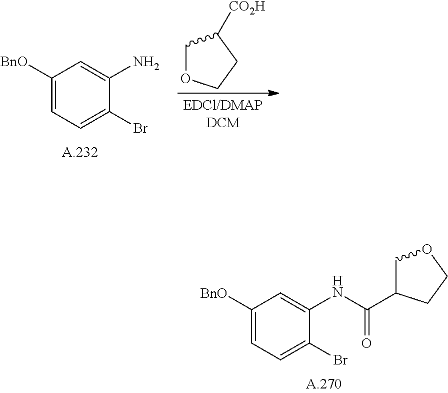

N-(5-(benzyloxy)-2-bromophenyl)tetrahydrofuran-3-carboxamide A.270 was prepared from 5-(benzyloxy)-2-bromoaniline A.232 and tetrahydro-3-furancarboxylic acid using chemistry similar to that described for compound A.234 (a white solid) ¹H NMR (400 MHz, DMSO-d₆) δ ppm 9.46 (1H, s), 7.53 (1H, d, J=8.6 Hz), 7.28-7.47 (6H, m), 6.84 (1H, dd, J=8.8, 2.9 Hz), 5.09 (2H, s), 3.92 (1H, t, J=8.0 Hz), 3.77 (2H, q, J=6.8 Hz), 3.70 (1H, q, J=7.0 Hz), 3.22-3.28 (1H, m), 2.09 (2H, q, J=6.8 Hz). LCMS-ESI (POS), M/Z, M+1: Found 376.1 and 378.1

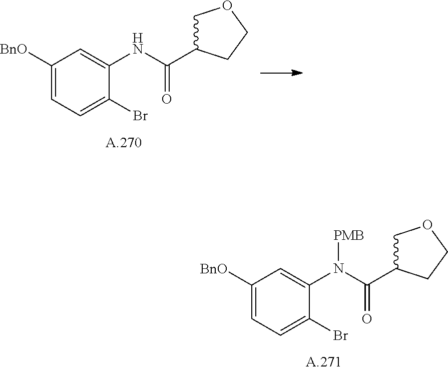

N-(5-(benzyloxy)-2-bromophenyl)-N-(4-methoxybenzyl)tetrahydrofuran-3-carboxamide A.271 (a colorless viscous liquid), was prepared from N-(5-(benzyloxy)-2-bromophenyl)tetrahydrofuran-3-carboxamide A.270 using chemistry similar to that described for compound A.235. ¹H NMR (400 MHz, DMSO-d₆) δ ppm 7.64 (1H, d, J=9.0 Hz), 7.29-7.42 (6H, m), 7.06 (2H, dd, J=8.6, 2.7 Hz), 7.00 (1H, dt, J=9.0, 3.5 Hz), 6.84 (2H, dd, J=8.6, 2.7 Hz), 6.61 (1H, t, J=3.1 Hz), 5.20 (1H, dd, J=14.3, 3.3 Hz), 4.98 (2H, s), 4.14 (1H, d, J=14.1 Hz), 3.65-3.80 (6H, m), 3.44-3.61 (2H, m), 2.59-2.72 (1H, m), 2.04-2.17 (1H, m). LCMS-ESI (POS), M/Z, M+1: Found 496.1 and 498.1.

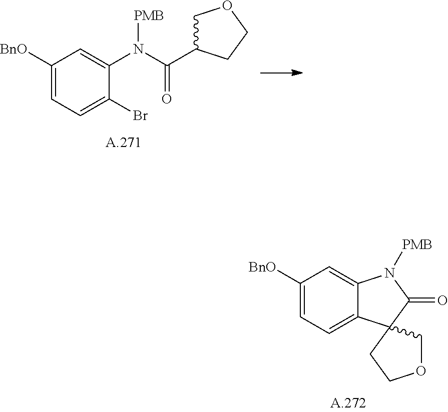

(rac)-6′6′-(benzyloxy)-1′-(4-methoxybenzyl)-4,5-dihydro-2H-spiro[furan-3,3′-indolin]-2′-one A.272 (a colorless viscous liquid) was prepared from N-(5-(benzyloxy)-2-bromophenyl)-N-(4-methoxybenzyl)tetrahydrofuran-3-carboxamide A.271 using chemistry similar to that described for compound A.236. ¹H NMR (400 MHz, DMSO-d₆) δ ppm 7.29-7.46 (6H, m), 7.18-7.26 (3H, m), 6.87 (2H, d, J=8.6 Hz), 6.62-6.68 (2H, m), 5.06 (2H, s), 4.81 (2H, s), 4.05-4.15 (2H, m), 3.85-3.90 (1H, m), 3.77 (1H, d, J=8.2 Hz), 3.71 (3H, s), 2.32 (1H, ddd, J=12.3, 7.4, 7.2 Hz), 2.08 (1H, dt, J=12.6, 6.4 Hz). LCMS-ESI (POS), M/Z, M+1: Found 416.2.

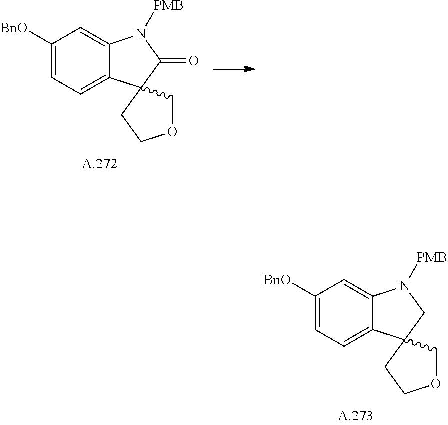

(rac)-6′-(benzyloxy)-1′-(4-methoxybenzyl)-4,5-dihydro-2H-spiro[furan-3,3′-indoline] A.273 (a colorless viscous liquid) was prepared from 6′-(benzyloxy)-1′-(4-methoxybenzyl)-4,5-dihydro-2H-spiro[furan-3,3′-indolin]-2′-one A.272 using chemistry similar to that described for compound A.237. LCMS-ESI (POS), M/Z, M+1: Found 402.2.

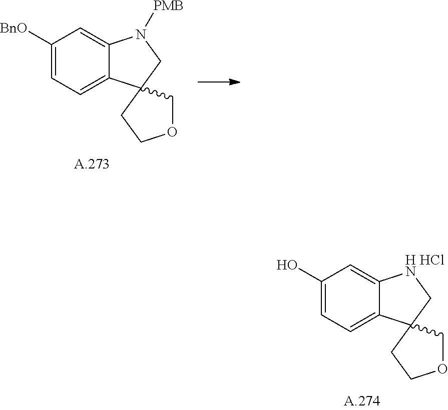

(rac)-1′,2′,4,5-Tetrahydrospiro[furan-3,3′-indol]-6′-ol hydrochloride A.274 (a colorless viscous liquid) was prepared from 6′-(benzyloxy)-1′-(4-methoxybenzyl)-4,5-dihydro-2H-spiro[furan-3,3′-indoline] A.273 using chemistry similar to that described for compound A.238. LCMS-ESI (POS), M/Z, M+1: Found 192.1.

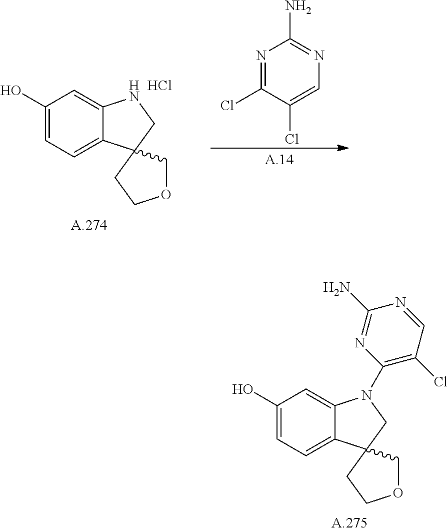

(rac)-1′-(2-Amino-5-chloro-4-pyrimidinyl)-1′,2′,4,5-tetrahydrospiro[furan-3,3′-indol]-6′-ol A.275 (an off-white solid) was prepared from 1′,2′,4,5-tetrahydrospiro[furan-3,3′-indol]-6′-ol hydrochloride A.274 and 4,5-dichloro-2-pyrimidinamine A.14 using chemistry similar to that described for compound A.239. ¹H NMR (400 MHz, DMSO-d₆) δ ppm 8.24 (1H, s), 7.19 (1 H, br. s.), 7.12 (1H, d, J=8.2 Hz), 6.52 (1H, dd, J=7.8, 2.0 Hz), 4.18-4.38 (2H, m), 3.91-4.02 (1H, m), 3.87 (1H, q, J=7.8 Hz), 3.78 (1H, d, J=8.2 Hz), 3.54 (1H, d, J=8.6 Hz), 2.05-2.23 (2H, m). LCMS-ESI (POS), M/Z, M+1: Found 319.1.

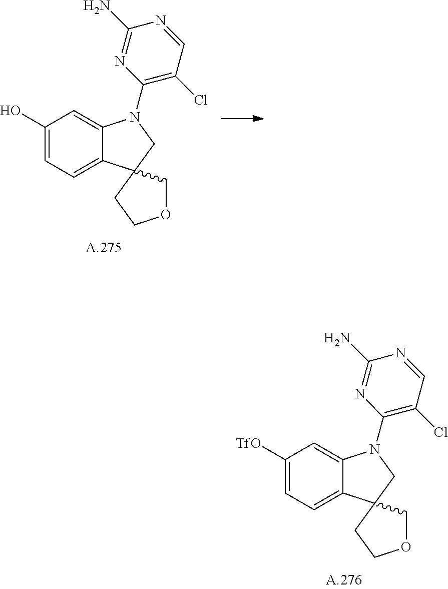

(rac)-1′-(2-amino-5-chloropyrimidin-4-yl)-4,5-dihydro-2H-spiro[furan-3,3′-indoline]-6′-yl trifluoromethanesulfonate A.276 (a colorless solid) was prepared from 1′-(2-amino-5-chloro-4-pyrimidinyl)-1′,2′,4,5-tetrahydrospiro[furan-3,3′-indol]-6′-ol A.275 using chemistry similar to that described for compound A.240. ¹H NMR (400 MHz, CHLOROFORM-d) δ ppm 8.12 (1H, s), 7.52 (1H, d, J=2.3 Hz), 7.23 (1H, d, J=8.6 Hz), 6.88 (1H, dd, J=8.2, 2.3 Hz), 5.07 (2H, br. s.), 4.38 (1H, d), 4.27 (1H, d), 4.07-4.15 (1H, m), 3.99-4.07 (1H, m), 3.88 (1 H, d), 3.79 (1H, d), 2.26-2.36 (1H, m), 2.14-2.22 (1H, m). LCMS-ESI (POS), M/Z, M+1: Found 451.0.

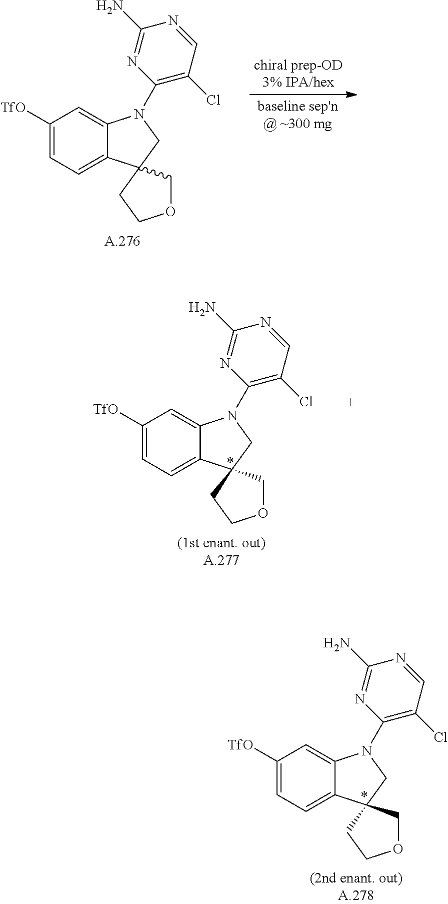

(3R*)-1′-(2-amino-5-chloropyrimidin-4-yl)-4,5-dihydro-2H-spiro[furan-3,3′-indoline]-6′-yl trifluoromethanesulfonate A.277 and (3S*)-1′-(2-amino-5-chloropyrimidin-4-yl)-4,5-dihydro-2H-spiro[furan-3,3′-indoline]-6′-yl trifluoromethanesulfonate A.278

Optical resolution of (rac)-1′-(2-amino-5-chloropyrimidin-4-yl)-4,5-dihydro-2H-spiro[furan-3,3′-indoline]-6′-yl trifluoromethanesulfonate was accomplished by the following method. Instrument: Agilent 1100 series. Column: OD-preparative (50 mm×500 mm). Solvents: 3% isopropanol in hexanes. Gradient: isochratic. Amount of sample per injection: 300 mg in methanol (6 mL). Separation quality: baseline separation. The first peak eluting off of the OD column provided (3R*)-1′-(2-amino-5-chloropyrimidin-4-yl)-4,5-dihydro-2H-spiro[furan-3,3′-indoline]-6′-yltrifluoromethanesulfonate A.277 as an off-white solid. The second peak furnished (3S*)-1′-(2-amino-5-chloropyrimidin-4-yl)-4,5-dihydro-2H-spiro[furan-3,3′-indoline]-6′-yl trifluoromethanesulfonate A.278 as an off-white solid. Stereochemistry arbitrarily assigned.

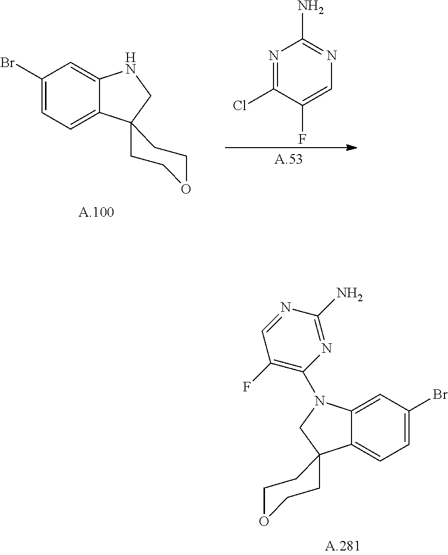

4-(6-Bromo-2′,3′,5′,6′-tetrahydrospiro[indole-3,4′-pyran]-1(2H)-yl)-5-fluoro-2-pyrimidinamine A.281 (an off-white solid) was prepared from 6-bromo-1,2,2′,3′,5′,6′-hexahydrospiro[indole-3,4′-pyran] (A.100) and 4-chloro-5-fluoro-2-pyrimidinamine A.53 using chemistry similar to that described for compound A.239. LCMS-ESI (POS), M/Z, M+1: Found 379.0 and 381.0

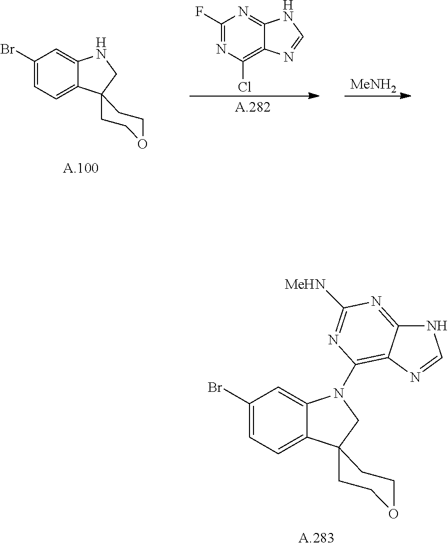

### 6-(6-Bromo-2′,3′,5′,6′-tetrahydrospiro[indole-3,4′-pyran]-1(2H)-yl)-N-methyl-9H-purin-2-amine (A.283)

To a stirred solution of 6-bromo-1,2,2′,3′,5′,6′-hexahydrospiro[indole-3,4′-pyran] (A.100) (0.24 g, 0.89 mmol) in t-butanol (12 mL) in a pressure vessel was added 6-chloro-2-fluoro-9H-purine A.282 (1.0 eq., 0.18 g) followed by Hunig's base (0.21 mL). The resulting mixture was stirred at 80° C. for 22 h. After cooling, methylamine (2 M solution in THF) was added to this mixture. The resulting mixture was stirred at 85° C. for 15 h. Upon workup, the volatiles were removed in vacuo and the residue was subjected to combi-flash column chromatography (methanol/dichloromethane) to give 6-(6-bromo-2′,3′,5′,6′-tetrahydrospiro[indole-3,4′-pyran]-1(2H)-yl)-N-methyl-9H-purin-2-amine A.283. LCMS-ESI (POS), M/Z, M+1: Found 415.0 and 417.0.

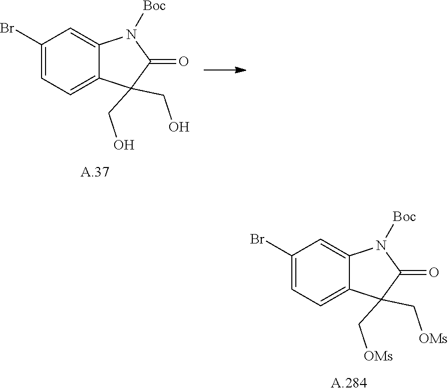

### tert-Butyl 6-bromo-3,3-bis(((methylsulfonyl)oxy)methyl)-2-oxo-2,3-dihydro-1H-indole-1-carboxylate (A.284)

To a stirred ice cooled suspension of tert-butyl 6-bromo-3,3-bis(hydroxymethyl)-2-oxo-2,3-dihydro-1H-indole-1-carboxylate A.37 (4.68 g, 12.6 mmol) in dichloromethane (100 mL) was added triethylamine (9.0 mL) through a syringe followed by MsCl (2.0 eq., 2.91 g) also through a syringe. The resulting mixture was stirred at 0° C. for 2.5 h as TLC showed completion. Upon workup, the volatiles were removed in vacuo and the residue was dissolved in dichloromethane and subjected to combi-flash column chromatography (ethyl acetate/hexanes) to give tert-butyl 6-bromo-3,3-bis(((methylsulfonyl)oxy)methyl)-2-oxo-2,3-dihydro-1H-indole-1-carboxylate A.284 (5.2 g, 79% yield) as an off-white solid. ¹H NMR (400 MHz, CHLOROFORM-d) δ ppm 8.14 (1H, d, J=2.0 Hz), 7.42 (1H, dd, J=8.0, 1.8 Hz), 7.31 (1H, d), 4.56 (2H, d, J=10.2 Hz), 4.45 (2H, d, J=10.2 Hz), 2.96 (6H, s), 1.66 (9H, s).

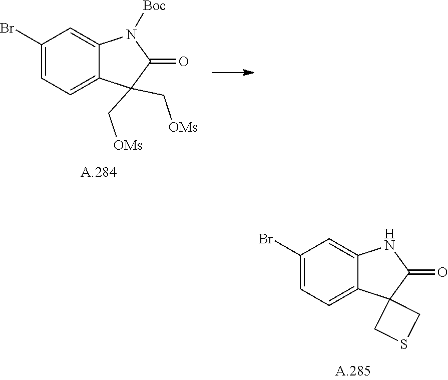

### 6-Bromospiro[indole-3,3′-thietan]-2(1H)-one A.285

Prepared by analogy to Roy, A., B. Achari, et al. (2006). Tetrahedron Lett. 47(23): 3875-3879. To a 100 mL single necked round bottom flask were placed tert-butyl 6-bromo-3,3-bis(((methylsulfonyl)oxy)methyl)-2-oxo-2,3-dihydro-1H-indole-1-carboxylate (4.51 g, 8.54 mmol) A.284 and Na₂S (1.5 eq., 1.0 g) followed by DMF (40 mL) at room temperature under N₂. The resulting mixture was stirred in an oil bath preheated at 105° C. for 38 h. Upon workup, the mixture was poured into ice and saturated NH₄Cl aqueous solution and extracted with ethyl acetate (2×). The combined organics were washed with brine (2×), dried over Na₂SO₄, and concentrated in vacuo. The residue was subjected to combi-flash column chromatography (ethyl acetate/hexanes) to give 6-bromospiro[indole-3,3′-thietan]-2(1H)-one A.285 (0.58 g, 25% yield) as an off-white solid. ¹H NMR (400 MHz, CHLOROFORM-d₆) δ ppm 7.94 (1H, d, J=7.8 Hz), 7.86 (1H, br. s.), 7.31 (1H, dd, J=8.0, 1.8 Hz), 7.07 (1H, d, J=1.6 Hz), 3.89 (2H, d, J=9.8 Hz), 3.12 (2H, d, J=9.8 Hz)

LCMS-ESI (NEG), M/Z, M−1: Found 268.0 AND 270.0.

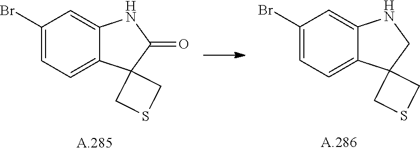

### 6-bromospiro[indoline-3,3′-thietane] A.286

To a stirred solution of 6-bromospiro[indole-3,3′-thietan]-2(1H)-one A.285 (0.58 g, 2.15 mmol) in toluene (85 mL) preheated at 80° C. was added Red-AL in toluene (3 M) (5.0 eq., 3.5 mL) under a nitrogen atmosphere. The resulting mixture was stirred at this temperature for 40 min. Upon workup, the mixture was cooled in an ice-salt bath before quenched with ice-cold aqueous 2 N NaOH. The mixture was further diluted with ice-cold aqueous 2 N NaOH and extracted with ethyl acetate (2×). The combined organics were dried over anhydrous Na₂SO₄ and concentrated in vacuo to give 6-bromo-1,2-dihydrospiro[indole-3,3′-thietane] A.286, LCMS-ESI (POS), M/Z, M+1: Found 256.0 and 258.0.

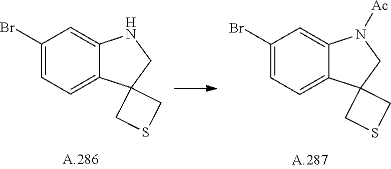

### 1-Acetyl-6-bromospiro[indoline-3,3′-thietane] A.287

To a stirred ice-cooled solution of crude 6-bromospiro[indoline-3,3′-thietane] A.286 (2.1 mmol, 100% yield assumed from the previous step), triethylamine (2.0 eq., 0.43 g), and DMAP (catalytic amount) in dichloromethane (30 mL) was added AcCl (2.0 eq., 0.34 g) under N₂. The resulting mixture was stirred at 0° C. for 5 min and at ambient temperature for 3 h. Upon workup, the mixture was poured into ice and 2 N HCl aqueous solution and extracted with ethyl acetate (2×). The combined organics were washed with brine (2×), dried over anhydrous Na₂SO₄ and concentrated in vacuo to give 1-acetyl-6-bromo-1,2-dihydrospiro[indole-3,3′-thietane] A.287. LCMS-ESI (POS), M/Z, M+1: Found 298.0 and 300.0.

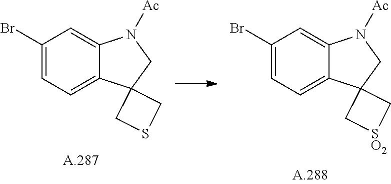

### 1-Acetyl-6-bromospiro[indoline-3,3′-thietane]1′,1′-dioxide A.288

To a stirred ice-cooled suspension of 1-acetyl-6-bromospiro[indoline-3,3′-thietane] A.287 (2.1 mmol, 100% yield assumed from the previous step) in a mixed solvent of methanol (40 mL), water (5 mL), and acetone (10 mL) was added a solution of oxone (2.0 eq., 2.6 g) in water (15 mL). The resulting mixture was stirred at ambient temperature for 20 h. Upon workup, the mixture was poured into ice and saturated NH₄Cl aqueous solution and extracted with ethyl acetate (2×). The combined organics were washed with brine (1×), dried over Na₂SO₄, and concentrated in vacuo to give 1-acetyl-6-bromospiro[indoline-3,3′-thietane]1′,1′-dioxide A.288. LCMS-ESI (POS), M/Z, M+1: Found 330.0 and 332.0.

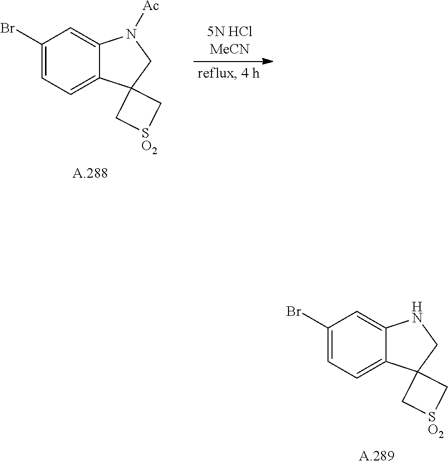

### 6-bromospiro[indoline-3,3′-thietane]1′,1′-dioxide A.289

A mixture of 1-acetyl-6-bromospiro[indoline-3,3′-thietane]1′,1′-dioxide A.288 (2.1 mmol, assumed from the previous step) and 5 N HCl (8 mL) in methanol (20 mL) was refluxed for 3 h. The volatiles were removed in vacuo to give 6-bromospiro[indoline-3,3′-thietane]1′,1′-dioxide A.289. LCMS-ESI (POS), M/Z, M+1: Found 288.0 and 290.0.

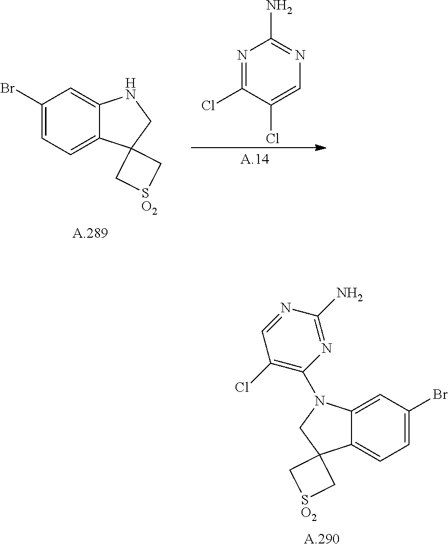

4-(6-Bromo-1′,1′-dioxidospiro[indole-3,3′-thietan]-1(2H)-yl)-5-chloro-2-pyrimidinamine A.290 (an off-white solid) was prepared from 6-bromospiro[indoline-3,3′-thietane]1′,1′-dioxide A.289 (2.1 mmol, assumed from the previous step) and 4,5-dichloro-2-pyrimidinamine A.14 (2.0 eq., 0.65 g) using chemistry similar to that described for compound A.239. [45% yield over 5 steps from 6-bromospiro[indole-3,3′-thietan]-2(1H)-one A.285]. ¹H NMR (400 MHz, DMSO-d₆) δ ppm 8.15 (1H, s), 7.59 (1H, d, J=8.2 Hz), 7.44 (1H, d, J=2.0 Hz), 7.21 (1 H, dd, J=8.0, 1.8 Hz), 6.76 (2H, s), 4.62 (2H, d), 4.43-4.57 (4H, m). LCMS-ESI (POS), M/Z, M+1: Found 414.9 and 416.8.

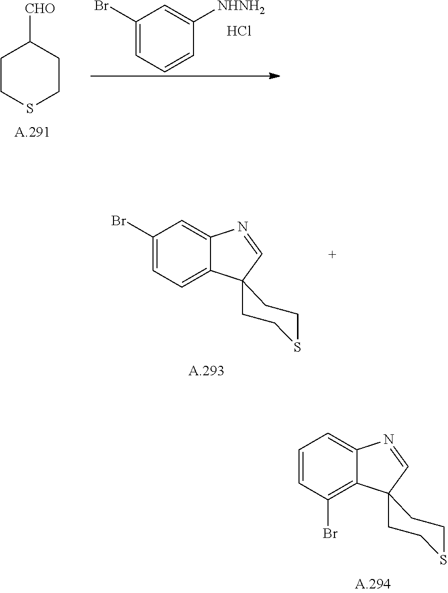

### 6-Bromo-2′,3′,5′,6′-tetrahydrospiro[indole-3,4′-thiopyran] (A.293) and 4-bromo-2′,3′,5′,6′-tetrahydrospiro[indole-3,4′-thiopyran]

Ref. Maligres, P. E., I. Houpis, et al. (1997). Tetrahedron 53(32): 10983-10992.

To a stirred ice-cooled solution of tetrahydro-2H-thiopyran-4-carbaldehyde A.291 (2.1 g, 16.1 mmol) in dichloromethane (30 mL) was added 3-bromophenylhydrazine hydrochloride (1.0 eq., 3.6 g) in one portion. The resulting mixture was stirred at 0° C. for 10 min before TFA (3.0 eq., 5.52 g) was dropwise added through a syringe. The resulting mixture was stirred at 0° C. for 10 min and then at ambient temperature for 4 h. Upon workup, the mixture was poured into ice and NH₃ water (28% commercial) and extracted with ethyl acetate (2×). The combined organics were washed with brine (2×), dried over anhydrous Na₂SO₄, and concentrated in vacuo. The residue was azeotroped with benzene (2×) to give a mixture (4.9 g) of 6-bromo-2′,3′,5′,6′-tetrahydrospiro[indole-3,4′-thiopyran] (A.293) and 4-bromo-2′,3′,5′,6′-tetrahydrospiro[indole-3,4′-thiopyran] (A.294) in a ratio of (1 to 1.3). LCMS-ESI (POS), M/Z, M+1: Found 282.0 and 284.0.

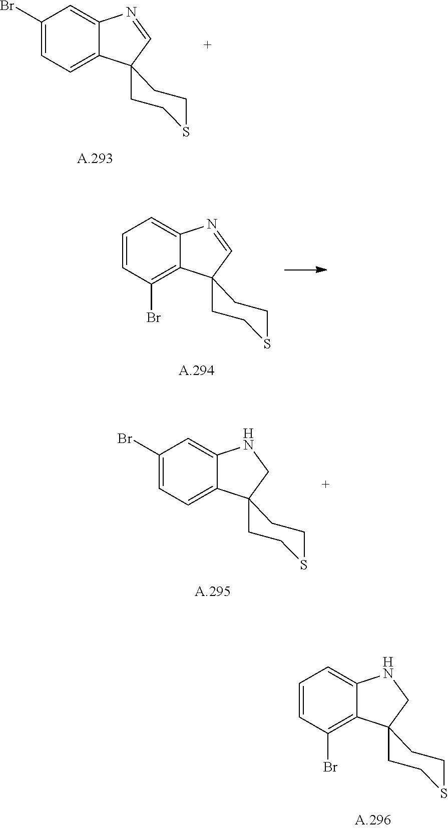

### 6-Bromo-1,2,2′,3′,5′,6′-hexahydrospiro[indole-3,4′-thiopyran] (A.295) and 4-bromo-1,2,2′,3′,5′,6′-hexahydrospiro[indole-3,4′-thiopyran] A.296

To a stirred solution of the mixture of 6-bromo-2′,3′,5′,6′-tetrahydrospiro[indole-3,4′-thiopyran] (A.293) and 4-bromo-2′,3′,5′,6′-tetrahydrospiro[indole-3,4′-thiopyran] (4.9 g) in toluene (80 mL) preheated at 80° C. was dropwise added Red-AL (15 mL) under N₂. The resulting mixture was stirred at 80° C. for 1.5 h. Upon workup, the mixture was thoroughly cooled in an ice bath before carefully quenched with ice cold 2 N NaOH. The mixture was further diluted with ice-cold aqueous 2 N NaOH and extracted with ethyl acetate (3×). The combined organics were dried over anhydrous Na₂SO₄ and concentrated in vacuo. The residue was subjected to combi-flash column chromatography (ethyl acetate/hexanes) to give 6-bromo-1,2,2′,3′,5′,6′-hexahydrospiro[indole-3,4′-thiopyran] (A.295) (1.65 g, 36% yield in 2 steps) and 4-bromo-1,2,2′,3′,5′,6′-hexahydrospiro[indole-3,4′-thiopyran] (A.296) (2.1 g, 46% yield in 2 steps) both as off-white solids.

6-bromo-1,2,2′,3′,5′,6′-hexahydrospiro[indole-3,4′-thiopyran] A.295. ¹H NMR (400 MHz, CHLOROFORM-d) δ ppm 6.89-6.94 (1H, m), 6.81-6.87 (1H, m), 6.75 (1H, d, J=1.2 Hz), 3.78 (1H, br. s.), 3.44 (2H, s), 2.69-2.83 (2H, m), 2.60 (2H, d, J=14.1 Hz), 1.87-2.07 (4H, m). LCMS-ESI (POS), M/Z, M+1: Found 284.0 and 286.0.

4-bromo-1,2,2′,3′,5′,6′-hexahydrospiro[indole-3,4′-thiopyran] A.296. ¹H NMR (400 MHz, CHLOROFORM-d) δ ppm 6.80-6.92 (2H, m), 6.55 (1H, d, J=7.4 Hz), 4.04 (0H, br. s.), 3.51 (2H, s), 2.75-2.90 (4H, m), 2.53-2.63 (2H, m), 1.88-1.99 (2H, m). LCMS-ESI (POS), M/Z, M+1: Found 284.0 and 286.0.

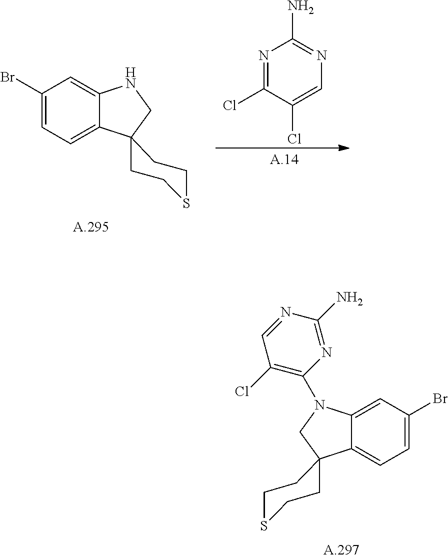

4-(6-Bromo-2′,3′,5′,6′-tetrahydrospiro[indole-3,4′-thiopyran]-1(2H)-yl)-5-chloro-2-pyrimidinamine A.297 (an off-white solid) was prepared from 6-bromo-1,2,2′,3′,5′,6′-hexahydrospiro[indole-3,4′-thiopyran] A.295 and 4,5-dichloro-2-pyrimidinamine A.14 using chemistry similar to that described for compound A.239. ¹H NMR (400 MHz, CHLOROFORM-d) δ ppm 8.08 (1H, s), 7.63 (1H, d, J=1.6 Hz), 7.12-7.20 (1H, m), 7.06 (1 H, d, J=7.8 Hz), 5.24 (2H, br. s.), 4.14 (2H, s), 2.80 (2H, ddd, J=14.6, 8.5, 6.3 Hz), 2.53-2.69 (2H, m), 1.93-2.04 (4H, m). LCMS-ESI (POS), M/Z, M+1: Found 411.0 and 412.9.

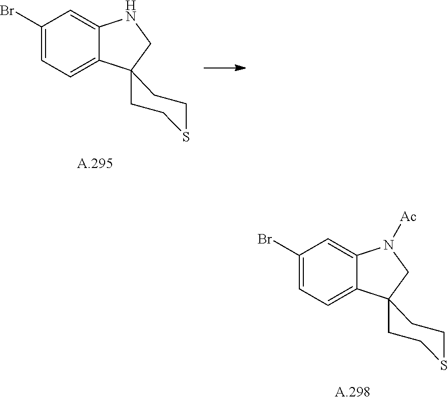

### 1-Acetyl-6-bromo-1,2,2′,3′,5′,6′-hexahydrospiro[indole-3,4′-thiopyran] A.298

To a stirred ice-cooled solution of 6-bromo-1,2,2′,3′,5′,6′-hexahydrospiro[indole-3,4′-thiopyran] A.295 (0.7 g, 2.46 mmol) and DMAP (catalytic amount) and triethylamine (1.5 eq., 0.37 g) in dichloromethane (25 mL) was added acetyl chloride through a syringe. The resulting mixture was stirred at 0° C. for 5 min and at ambient temperature for 1.5 h. Upon workup, the mixture was poured into ice and 2 NHCl aqueous solution and extracted with ethyl acetate (2×). The combined organics were washed with brine (1×), saturated NaHCO₃ aqueous solution (1×), brine (1×), and dried over anhydrous Na₂SO₄. The residue was subjected to combi-flash column chromatography (ethyl acetate/hexanes) to give 1-acetyl-6-bromo-1,2,2′,3′,5′,6′-hexahydrospiro[indole-3,4′-thiopyran] A.298 (0.8 g, quantitative yield) as an off-white solid.

¹H NMR (400 MHz, DMSO-d₆) δ ppm 8.20 (1H, s), 7.21-7.27 (1H, m), 7.15-7.21 (1H, m), 3.99 (2H, s), 2.74-2.90 (2H, m), 2.52-2.60 (2H, m), 2.22 (3H, s), 1.81-1.93 (4H, m). LCMS-ESI (POS), M/Z, M+1: Found 326.0 AND 328.0.

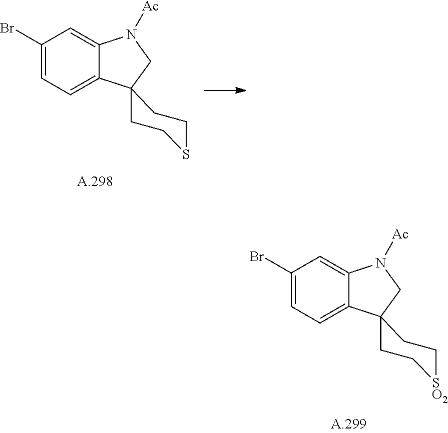

1-Acetyl-6-bromo-1,2,2′,3′,5′,6′-hexahydrospiro[indole-3,4′-thiopyran] 1′,1′-dioxide A.299 (a white solid) was prepared from 1-acetyl-6-bromo-1,2,2′,3′,5′,6′-hexahydrospiro[indole-3,4′-thiopyran] A.298 using chemistry similar to that described for compound A.288.

1H NMR (400 MHz, DMSO-d6) δ ppm 8.20 (1H, s), 7.24-7.31 (1H, m), 7.21 (1H, dd, J=8.0, 1.8 Hz), 4.18 (2H, s), 3.34-3.47 (2H, m), 3.10 (2H, d, J=12.9 Hz), 2.17-2.34 (5H, m), 1.96-2.08 (2H, m). LCMS-ESI (POS), M/Z, M+1: Found 358.0 and 360.0.

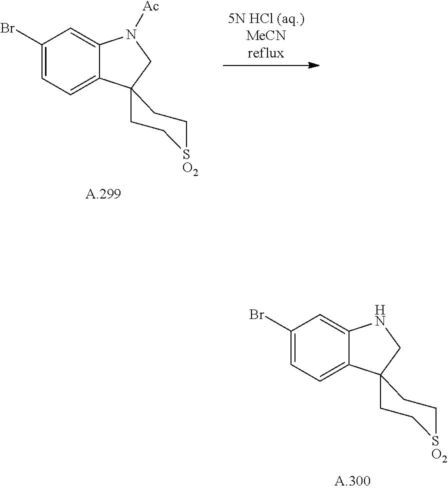

6-Bromo-1,2,2′,3′,5′,6′-hexahydrospiro[indole-3,4′-thiopyran] 1′,1′-dioxide A.300 (an off-white solid) was prepared from 1-acetyl-6-bromo-1,2,2′,3′,5′,6′-hexahydrospiro[indole-3,4′-thiopyran]1′,1′-dioxide A.299 using chemistry similar to that described for compound A.289. LCMS-ESI (POS), M/Z, M+1: Found 316.0 and 318.0.

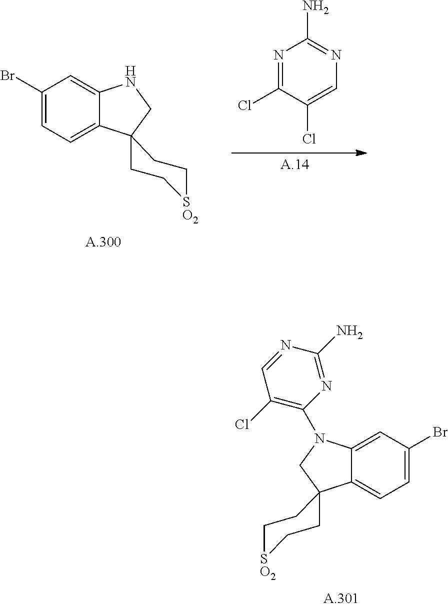

4-(6-Bromo-1′,1′-dioxido-2′,3′,5′,6′-tetrahydrospiro[indole-3,4′-thiopyran]-1(2H)-yl)-5-chloro-2-pyrimidinamine A.301 was prepared from 6-bromo-1,2,2′,3′,5′,6′-hexahydrospiro[indole-3,4′-thiopyran] 1′,1′-dioxide A.300 and 4,5-dichloro-2-pyrimidinamine A.14 using chemistry similar to that described for compound A.239 in 92% as an off-white solid. ¹H NMR (400 MHz, DMSO-d₆) δ ppm 8.15 (1H, s), 7.19-7.31 (2H, m), 7.04-7.12 (1 H, m), 6.73 (2H, s), 4.22 (2H, s), 3.33-3.41 (2H, m), 3.02-3.14 (2H, m), 2.21-2.30 (2H, m), 2.01-2.10 (2H, m). LCMS-ESI (POS), M/Z, M+1: Found 442.9 and 445.0.

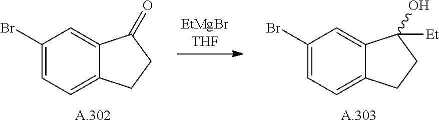

### 6-Bromo-1-ethyl-2,3-dihydro-1H-inden-1-ol

Ref. Shindo, M., Y. Sato, et al. (2001). J. Org. Chem. 66(23): 7818-7824.

To a stirred solution of EtMgBr in THF (1.0 M, commercial) (2.0 eq., 50 mL) was dropwise added at room temperature a solution of 6-bromoindanone A.302 (5.0 g, 23.69 mmol) in THF (45 mL). The resulting mixture was stirred at ambient temperature overnight. Upon workup, the mixture was cooled in an ice water before carefully quenched with saturated NH₄Cl aqueous solution followed by extraction with ethyl acetate (2×). The combined organics were washed with brine (2×), dried over anhydrous Na₂SO₄, and concentrated in vacuo to give 6-bromo-1-ethyl-2,3-dihydro-1H-inden-1-ol A.303 as a colorless liquid, ¹H NMR (500 MHz, DMSO-d₆) δ ppm 7.37 (1H, d, J=2.0 Hz), 7.35 (1H, dd, J=8.1, 2.0 Hz), 7.16 (1H, d, J=8.1 Hz), 5.01 (1H, s), 2.77-2.88 (1H, m), 2.63-2.72 (1H, m), 2.12 (1H, ddd, J=12.7, 8.3, 4.2 Hz), 1.91-1.98 (1H, m), 1.68-1.77 (1H, m), 1.54-1.65 (1H, m), 0.83 (3H, t, J=7.5 Hz).

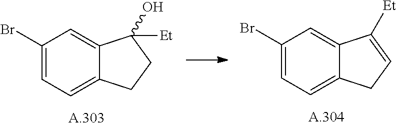

### 5-Bromo-3-ethyl-1H-indene A.304

To a stirred solution of 6-bromo-1-ethyl-2,3-dihydro-1H-inden-1-ol (A.303) (23.69 mmol, assumed from the previous step) in dichloromethane (60 mL) was added TFA (0.5 mL) at room temperature. The resulting mixture was stirred at ambient temperature for 24 h. Upon workup, the mixture was concentrated in vacuo. The residue was subjected to combi-flash column chromatography (hexanes) to give 5-bromo-3-ethyl-1H-indene A.304 (4.7 g, 90% in 2 steps) as a colorless liquid. ¹H NMR (400 MHz, CHLOROFORM-d) δ ppm 7.48 (1H, s), 7.31 (2H, s), 6.25 (1H, s), 3.29 (2H, d, J=2.2 Hz), 2.48-2.58 (2H, m), 1.29 (3H, t, J=7.3 Hz).

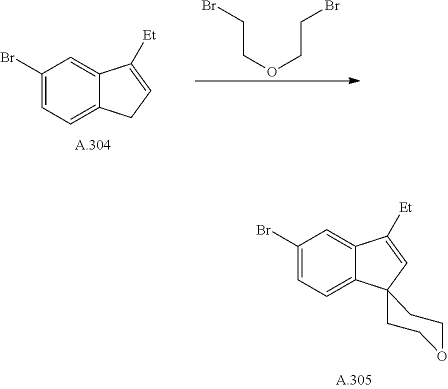

### 5-Bromo-3-ethyl-2′,3′,5′,6′-tetrahydrospiro[indene-1,4′-pyran] A.305

To a stirred ice-cooled solution of 5-bromo-3-ethyl-1H-indene A.304 (4.7 g, 21.07 mmol) in THF (20 mL) was dropwise added NaHMDS (2 M in THF) (2.0 eq., 21.1 mL) through a syringe under N₂ over a period of 10 min. The resulting mixture was stirred at 0° C. for 50 min and then transferred through a cannula to a stirred ice-cooled solution of bis(2-bromoethyl)ether in THF (25 mL) under N₂ over a period of 35 min. The resulting mixture was stirred at 0° C. for 35 min, then at ambient temperature for 2 days. Upon workup, the mixture was cooled thoroughly in an ice bath before quenched with saturated NH₄Cl aqueous solution and extracted with ethyl acetate (2×). The combined organics were washed with brine (2×) and dried over anhydrous Na₂SO₄. The residue after concentration in vacuo was subjected to combi-flash column chromatography (ethyl acetate/hexanes) to give 5-bromo-3-ethyl-2′,3′,5′,6′-tetrahydrospiro[indene-1,4′-pyran] A.305 (1.85 g, 30% yield) as a thick oil. ¹H NMR (400 MHz, CHLOROFORM-d) δ ppm 7.41 (1H, d, J=1.5 Hz), 7.35 (1H, dd, J=7.9, 1.6 Hz), 7.23 (1H, d, J=7.7 Hz), 6.64 (1H, s), 4.07 (2H, ddd, J=11.7, 4.0, 2.6 Hz), 3.77 (2H, td, J=11.9, 1.8 Hz), 2.50 (2H, qd, J=7.4, 1.3 Hz), 2.15 (2H, td, J=12.8, 4.4 Hz), 1.22-1.31 (5H, m). LCMS-ESI (POS), M/Z, M+1: Found 293.0 and 295.0.

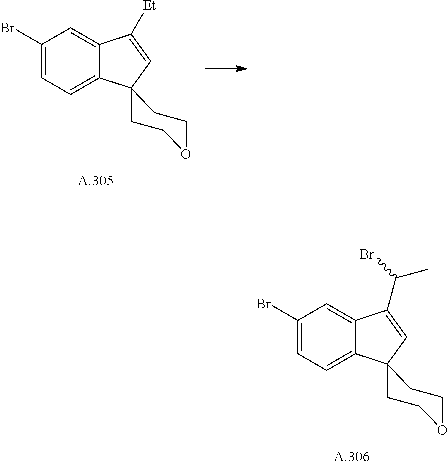

5-Bromo-3-(1-bromoethyl)-2′,3′,5′,6′-tetrahydrospiro[indene-1,4′-pyran] A.306 by analogy to Pettit, W. A. and J. W. Wilson (1977). J. Amer. Chem. Soc. 99(19): 6372-6379.

To a stirred solution of 5-bromo-3-ethyl-2′,3′,5′,6′-tetrahydrospiro[indene-1,4′-pyran] A.305 (1.2 g, 4.09 mmol) in CCl₄ (50 mL) was added NBS (1.02 eq., 0.75 g) followed by (BzO)₂ (catalytic amount). The resulting mixture was heated at reflux for 6 h. The volume of the reaction mixture was reduced. After cooling, the mixture was subjected to combi-flash column chromatography (ethyl acetate/hexanes) to give 5-bromo-3-(1-bromoethyl)-2′,3′,5′,6′-tetrahydrospiro[indene-1,4′-pyran] A.306 (0.86 g, 56% yield) as a thick oil. ¹H NMR (400 MHz, CHLOROFORM-d₆) δ ppm 7.65 (1H, d, J=1.8 Hz), 7.41 (1H, dd, J=7.9, 1.6 Hz), 7.25 (1H, d, J=8.1 Hz), 6.96 (1H, s), 5.09-5.15 (1H, m), 4.03-4.12 (2H, m), 3.76 (2H, td, J=11.9, 2.2 Hz), 2.11-2.22 (2H, m), 2.09 (3H, d, J=7.0 Hz), 1.29-1.37 (2H, m).

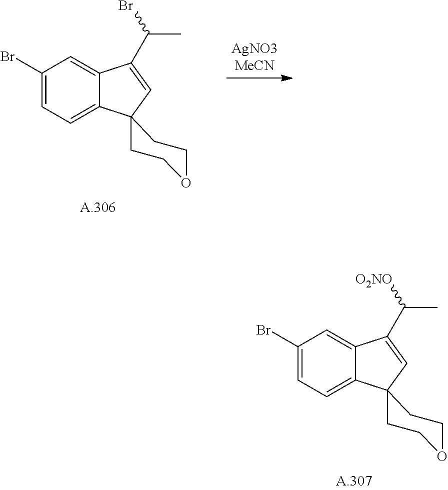

### 1-(5-Bromo-2′,3′,5′,6′-tetrahydrospiro[indene-1,4′-pyran]-3-yl)ethyl nitrate A.307

Ref.: By analogy to Kinoshita,T., Takaishi, Y. et al. (1992) J. Heterocycl. Chem._{—}29: 741-747

To a stirred solution of 5-bromo-3-(1-bromoethyl)-2′,3′,5′,6′-tetrahydrospiro[indene-1,4′-pyran] A.306 (0.95 g, 2.55 mmol) in acetonitrile (30 mL) at reflux was added a solution of silver nitrate (1.5 eq., 0.66 g) in acetonitrile (5 mL). The resulting mixture was heated at reflux for 2 h. After cooling, the mixture was filtered through a layer of celite and the filter cake was washed with ethyl acetate. The filtrate was concentrated in vacuo to give 1-(5-bromo-2′,3′,5′,6′-tetrahydrospiro[indene-1,4′-pyran]-3-yl)ethyl nitrate A.307 (1.0 g) as a thick film. ¹H NMR (500 MHz, CHLOROFORM-d) δ ppm 7.50 (1H, d, J=1.7 Hz), 7.42 (1H, dd, J=7.8, 1.7 Hz), 7.25-7.28 (1H, m), 6.98 (0H, s), 6.02 (0H, q, J=6.4 Hz), 4.04-4.12 (2H, m), 3.68-3.81 (2 H, m), 2.14-2.23 (2H, m), 1.71 (3H, d, J=6.6 Hz), 1.23-1.32 (2H, m). LCMS-ESI (POS), M/Z, M+1: Found 354.0 and 356.0.

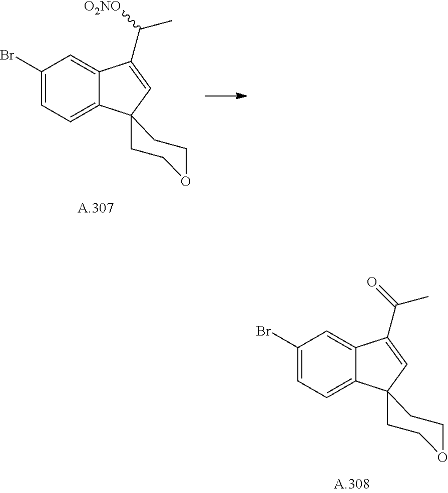

### 1-(5-Bromo-2′,3′,5′,6′-tetrahydrospiro[indene-1,4′-pyran]-3-yl)ethanone A.308

Ref. Using methods found in (1) Abdourazak, A. H., Z. Marcinow, et al. (1994). Tetrahedron Lett. 35(23): 3857-3860; (2) Kornblum, N. and H. W. Frazier (1966). J. Amer. Chem. Soc. 88(4): 865-866.

To a stirred solution of the crude 1-(5-bromo-2′,3′,5′,6′-tetrahydrospiro[indene-1,4′-pyran]-3-yl)ethyl nitrate A.307 (2.55 mmol, 100% yield assumed from the previous step) in DMSO (25 mL) was added at room temperature NaOAc (0.27 eq., 56 mg) followed by 2 drops of water through a pipette. The resulting mixture was stirred at 95° C. for 2 h at ambient temperature overnight. Upon workup, the mixture was poured into ice and brine and extracted with ethyl acetate (2×). The combined organics were washed with brine (1×), dried over anhydrous Na₂SO₄, and concentrated in vacuo. The residue was subjected to combi-flash column chromatography (ethyl acetate/hexanes) to give 1-(5-bromo-2′,3′,5′,6′-tetrahydrospiro[indene-1,4′-pyran]-3-yl)ethanone A.308 (0.3 g, 43% yield over 2 steps) as a light yellow solid. ¹H NMR (400 MHz, CHLOROFORM-d) δ ppm 8.33 (1H, d, J=1.8 Hz), 7.67 (1H, s), 7.43 (1H, dd, J=8.1, 1.8 Hz), 7.26 (1H, m), 4.09-4.18 (2H, m), 3.81 (2H, td, J=11.9, 2.2 Hz), 2.54 (3 H, s), 2.22 (2H, ddd, J=13.8, 12.0, 4.6 Hz). LCMS-ESI (POS), M/Z, M+1: Found 307.0 and 309.0.

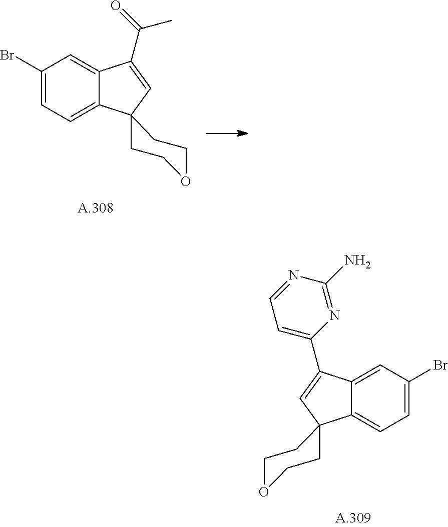

### 4-(5-Bromo-2′,3′,5′,6′-tetrahydrospiro[indene-1,4′-pyran]-3-yl)-2-pyrimidinamine A.309

1-(5-Bromo-2′,3′,5′,6′-tetrahydrospiro[indene-1,4′-pyran]-3-yl)ethanone A.308 (85 mg, 0.28 mmol) was treated neat with t-BuOCH(NMe₂)₂ (2.3 eq., 0.11 g) at 105° C. under N₂ for 3 h. The mixture was cooled before n-PrOH (8 mL), guanidine hydrochloride (5.0 eq., 0.13 g), and MeONa (5.53 M in methanol) (3.0 eq., 0.15 mL) were added sequentially. The resulting mixture was stirred at 95° C. for 3 h. Upon workup, the mixture was cooled and poured into ice and saturated NaHCO₃ aqueous solution and extracted with ethyl acetate (1×). The organic layer was washed with brine (2×), dried over anhydrous Na₂SO₄, and concentrated in vacuo to give 4-(5-bromo-2′,3′,5′,6′-tetrahydrospiro[indene-1,4′-pyran]-3-yl)-2-pyrimidinamine A.309 (0.1 g, quant. yield) as a light yellow solid. ¹H NMR (500 MHz, DMSO-d₆) δ ppm 8.38 (1H, d, J=5.1 Hz), 8.27 (1H, d, J=2.0 Hz), 7.53 (1H, s), 7.45 (1H, dd, J=7.8, 2.0 Hz), 7.23-7.33 (2H, m), 6.99 (0H, d, J=5.1 Hz), 5.17 (2H, br. s.), 4.07-4.17 (2H, m), 3.83 (2H, td, J=11.9, 1.8 Hz), 2.24 (2H, ddd, J=13.4, 12.4, 4.5 Hz), 1.39 (2H, dd, J=13.6, 1.3 Hz). LCMS-ESI (POS), M/Z, M+1: Found 358.0 and 360.1.

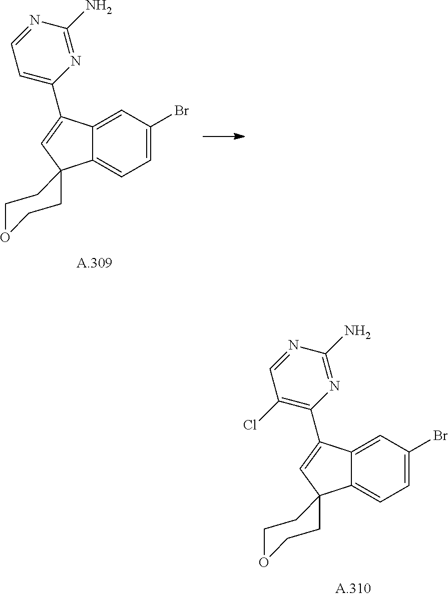

### 4-(5-Bromo-2′,3′,5′,6′-tetrahydrospiro[indene-1,4′-pyran]-3-yl)-5-chloro-2-pyrimidinamine A.310

To a stirred solution of 4-(5-bromo-2′,3′,5′,6′-tetrahydrospiro[indene-1,4′-pyran]-3-yl)-2-pyrimidinamine A.309 (0.1 g, 0.277 mmol) in acetonitrile (18 mL) was added NCS (1.0 eq., 40 mg) in one portion. The resulting mixture was stirred at reflux for 5.5 h and then concentrated in vacuo. The residue was subjected to combi-flash column chromatography (ethyl acetate/hexanes) to give 4-(5-bromo-2′,3′,5′,6′-tetrahydrospiro[indene-1,4′-pyran]-3-yl)-5-chloro-2-pyrimidinamine A.310 (65 mg, 60% yield) as an off-white solid. ¹H NMR (400 MHz, CHLOROFORM-d) δ ppm 8.38 (1H, s), 7.82 (1H, d, J=1.8 Hz), 7.61 (1H, s), 7.44 (1 H, dd, J=7.9, 1.6 Hz), 7.30 (1H, d, J=8.1 Hz), 5.15 (2H, br. s.), 4.07-4.18 (2H, m), 3.82 (2 H, td, J=12.1, 1.8 Hz), 2.20-2.33 (2H, m), 1.44 (2H, dd, J=13.5, 1.5 Hz). LCMS-ESI (POS), M/Z, M+1: Found 392.0 and 394.0.

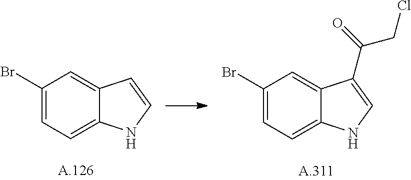

### 1-(5-Bromo-1H-indol-3-yl)-2-chloroethanone A.311

To stirred ice-cooled suspension of AlCl₃ (2.0 eq., 5.5 g) in dichloromethane (30 mL) was slowly added through a syringe chloroacetyl chloride (1.2 eq., 2.82 g) under a nitrogen atmosphere. This was followed by a solution of 5-bromoindole A.126 (4.0 g, 20.4 mmol) in dichloromethane (35 mL). The resulting mixture was stirred at 0° C. for 10 min and at ambient temperature for 4 h. Upon workup, the mixture was poured into stirred ice and water and extracted with ethyl acetate (2×). The combined organics were washed with brine (2×), dried over Na₂SO₄, and concentrated in vacuo. The residue was triturated with ethyl acetate/hexanes to give 1-(5-bromo-1H-indol-3-yl)-2-chloroethanone A.311 (3.4 g, 61% yield) as an off-white solid. ¹H NMR (400 MHz, DMSO-d₆) δ ppm 12.33 (1H, br. s.), 8.48 (1H, d, J=3.3 Hz), 8.29 (1H, d, J=1.8 Hz), 7.49 (1H, d, J=8.8 Hz), 7.38 (1H, dd, J=8.6, 2.0 Hz), 4.88 (2H, s). LCMS-ESI (POS), M/Z, M+1: Found 271.9 and 273.9.

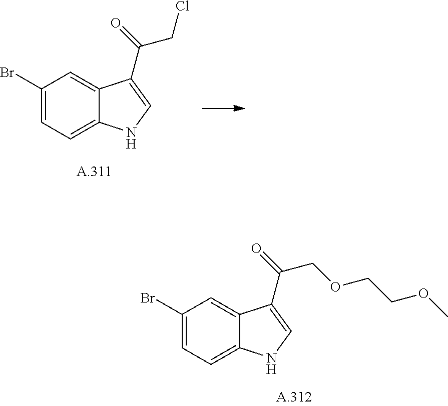

### 1-(5-Bromo-1H-indol-3-yl)-2-(2-methoxyethoxy)ethanone A.312

To a stirred ice-cooled suspension of NaH (commercial, 60% dispersion in mineral oil) (2.2 eq., 1.29 g) in DMF (25 mL) was added dropwise 2-methoxyethanol (2.0 eq., 2.24 g) through a syringe. The resulting mixture was stirred at 0° C. for 10 min before the ice bath was removed and a solution of 1-(5-bromo-1H-indol-3-yl)-2-chloroethanone A.311 (4.0 g, 14.7 mmol) in DMF (30 mL) was added through a syringe. The resulting mixture was stirred at ambient temperature for 2.5 h. Upon workup, the mixture was poured into ice and saturated NH₄Cl aqueous solution and extracted with ethyl acetate. The combined organics were washed with brine (2×), dried over Na₂SO₄, and concentrated in vacuo. The residue was triturated with ethyl acetate/hexanes to give 1-(5-bromo-1H-indol-3-yl)-2-(2-methoxyethoxy)ethanone A.312 (4.2 g, 92% yield) as an off-white solid. ¹H NMR (400 MHz, DMSO-d₆) δ ppm 12.15 (1H, br. s.), 8.42 (1H, s), 8.31 (1H, d, J=1.8 Hz), 7.47 (1H, d, J=8.4 Hz), 7.36 (1H, dd, J=8.6, 2.0 Hz), 4.56 (2H, s), 3.63-3.71 (2H, m), 3.52 (2H, dd, J=5.5, 3.7 Hz), 3.27 (3H, s). LCMS-ESI (POS), M/Z, M+1: Found 312.0 and 314.0.

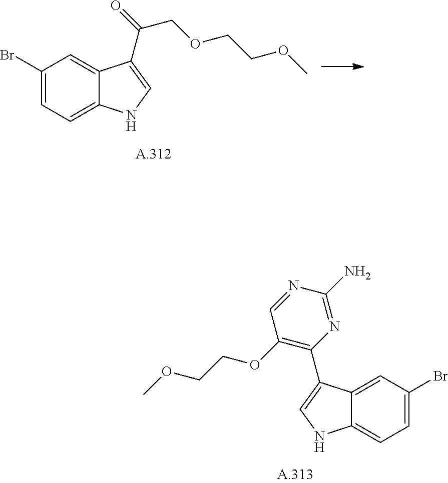

### 4-(5-Bromo-1H-indol-3-yl)-5-(2-methoxyethoxy)-2-pyrimidinamine A.313

1-(5-Bromo-1H-indol-3-yl)-2-(2-methoxyethoxy)ethanone A.312 (4.0 g, 12.8 mmol) was treated neat with t-BuOCH(NMe₂)₂ (2.0 eq., 4.47 g) at 105° C. under N₂ for 1.5 h. The mixture was cooled before n-PrOH (35 mL), guanidine hydrochloride (5.0 eq., 6.2 g), and MeONa (5.53 M in methanol) (3.0 eq., 7.5 mL) were added sequentially. The resulting mixture was stirred at 95° C. for 24 h. Upon workup, the mixture was cooled and poured into ice and saturated NaHCO₃ aqueous solution and extracted with ethyl acetate (3×). The combined organics were washed with brine (2×), dried over anhydrous Na₂SO₄, and concentrated in vacuo to give 4-(5-bromo-1H-indol-3-yl)-5-(2-methoxyethoxy)-2-pyrimidinamine A.313 (4.5 g, 97% yield) as a light yellow solid. ¹H NMR (400 MHz, DMSO-d₆) δ ppm 11.84 (1H, s), 8.94 (1H, d, J=1.8 Hz), 8.44 (1H, s), 8.05 (1H, s), 7.41 (1H, d, J=8.4 Hz), 7.30 (1H, dd, J=8.6, 2.0 Hz), 6.14 (2H, s), 4.12-4.21 (2H, m), 3.71-3.77 (2H, m), 3.36 (3H, s). LCMS-ESI (POS), M/Z, M+1: Found 363.0 and 364.9.

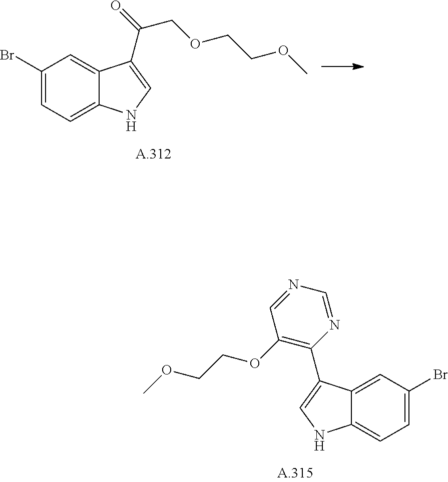

5-Bromo-3-(5-(2-methoxyethoxy)-4-pyrimidinyl)-1H-indole A.315 was prepared using chemistry similar to that described for compound A.313 except that imidoformamide hydrochloride was used in place of guanidine hydrochloride. ¹H NMR (400 MHz, DMSO-d₆) δ ppm 12.01 (1H, br. s.), 8.95 (1H, d, J=1.8 Hz), 8.83 (1H, s), 8.52 (1H, s), 8.47 (1H, s), 7.47 (1H, d, J=8.4 Hz), 7.34 (1H, dd, J=8.6, 2.0 Hz), 4.38-4.48 (2H, m), 3.81-3.87 (2H, m), 3.39 (3H, s). LCMS-ESI (POS), M/Z, M+1: Found 348.0 and 350.0.

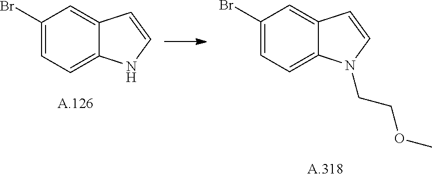

### 5-Bromo-1-(2-methoxyethyl)-1H-indole (A.318)

A stirred mixture of 5-bromoindole A.126 (3.0 g, 0.015 mol), 50% sodium hydroxide aqueous solution (30 mL), KI (catalytic amount), and tetrabutylammonium hydroxide (0.5 g, 10 mol %) in benzene (80 mL) was heated in an oil bath to reflux, at which time 1-bromo-2-methoxyethane (3.0 mL, 0.030 mol) was added through a syringe. The resulting mixture was heated at reflux for 1 h, at which time about 70% conversion was observed and more 1-bromo-2-methoxyethane (1.0 mL) was added. The reaction continued for another hour at reflux and then stirred at 70° C. overnight. Upon workup, the mixture was poured into ice water and extracted with benzene (2×). The combined organics were washed with brine (2×), dried over anhydrous sodium sulfate, and concentrated in vacuo to give 5-bromo-1-(2-methoxyethyl)-1H-indole (A.318) in nearly quantitative yield as a nearly colorless oil. ¹H NMR (400 MHz, DMSO-d₆) δ 7.72 (d, J=1.5 Hz, 1H), 7.48 (d, J=9.0 Hz, 1H), 7.40 (d, J=3.0 Hz, 1H), 7.23 (dd, =9.0, 1.5 Hz, 1H), 6.42 (d, J=3.0 Hz, 1H), 4.33 (t, J=5.0 Hz, 2H), 3.64 (t, J=5.0 Hz, 2H), 3.20 (s, 3H). LCMS-ESI (POS), M/Z, M+1: Found 254.0 and 256.0.

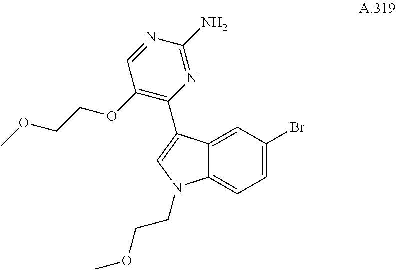

### 4-(5-bromo-1-(2-methoxyethyl)-1H-indol-3-yl)-5-(2-methoxyethoxy)-2-pyrimidinamine

A.319 was prepared in analogy to the procedure described for compound A.313 starting from compound A.318. ¹H NMR (400 MHz, DMSO-d₆) δ 8.97 (d, J=2.0 Hz, 1H), 8.47 (s, 1H), 8.08 (s, 1H), 7.58 (d, =8.7 Hz, 1H), 7.36 (dd, J=8.7, 2.0 Hz, 1H), 6.19 (s, 2H), 4.42 (t, J=5.1 Hz, 2H), 4.17 (t, J=4.4 Hz, 2H), 3.76 (t, J=4.4 Hz, 2H), 3.71 (t, J=5.1 Hz, 2H), 3.39 (s, 3H), 3.25 (s, 3H). LCMS-ESI (POS), M/Z, M+1: Found 421.0 and 423.1.

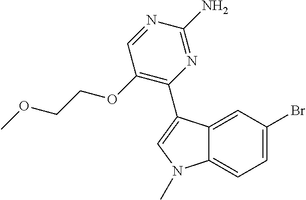

### 4-(5-bromo-1-methyl-1H-indol-3-yl)-5-(2-methoxyethoxy)-2-pyrimidinamine A.317

A.317 was prepared in analogy to the procedure described for compound A.313 starting from 5-bromo-1-methyl-1H-indole A.316.

¹H NMR (400 MHz, DMSO-d₆) δ 8.97 (d, J=2.0 Hz, 1H), 8.44 (s, 1H), 8.08 (s, 1H), 7.58 (d, =8.7 Hz, 1H), 7.36 (dd, J=8.7, 2.0 Hz, 1H), 6.19 (s, 2H), 4.17 (t, J=4.5 Hz, 2H), 3.88 (s, 3H), 3.76 (t, J=4.5 Hz, 2H), 3.40 (s, 3H). LCMS-ESI (POS), M/Z, M+1: Found 377.0 and 379.0.

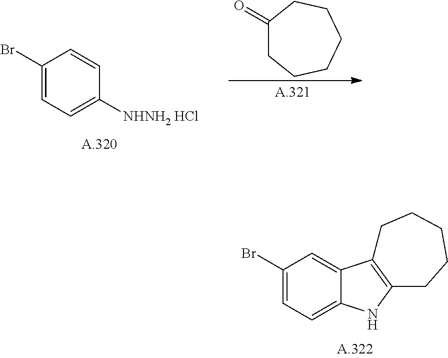

### 2-Bromo-5,6,7,8,9,10-hexahydrocyclohepta[b]indole A.322

A suspension of (4-bromophenyl)hydrazine hydrochloride A.320 (10 g, 44.7 mmol) in glacial acetic acid (150 mL) was added portion-wise into a stirred solution of cycloheptanone A.321 (1.0 eq., 5.02 g) in glacial acetic acid (50 mL) preheated at 130° C. The resulting mixture was heated to reflux (145° C.) for 2 h. Upon workup, the mixture was cooled and diluted with water while being stirred vigorously. The precipitate was collected by vacuum filtration and the filter cake was washed with water and air-dried to give 2-bromo-5,6,7,8,9,10-hexahydrocyclohepta[b]indole A.322 (11.5 g, 98% yield) as an off-white solid. ¹H NMR (500 MHz, DMSO-d₆) δ ppm 10.89 (1H, br. s.), 7.52 (1H, d, J=1.5 Hz), 7.17 (1H, d, J=8.3 Hz), 7.04 (1H, dd, J=8.6, 1.7 Hz), 2.75-2.86 (2H, m), 2.66-2.73 (2H, m), 1.77-1.88 (2H, m), 1.59-1.72 (4H, m). LCMS-ESI (POS), M/Z, M+1: Found 264.0 and 266.0.

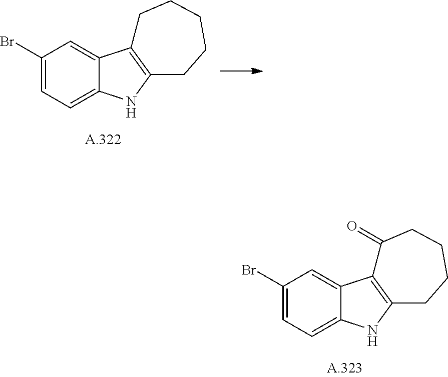

### 2-Bromo-6,7,8,9-tetrahydrocyclohepta[b]indol-10(5H)-one A.322

To a stirred ice-cooled solution of 2-bromo-5,6,7,8,9,10-hexahydrocyclohepta[b]indole A.322 (7.0 g, 26.5 mmol) in a mixed solvent consisting of THF and water (100 mL, v/v, 9:1) was added DDQ in two portions over a period of 15 min. The resulting mixture was stirred at 0° C. for 30 min and at ambient temperature for 2 h. Upon workup, water was added followed by saturated NaHCO₃ aqueous solution. The mixture was vigorously stirred and then allowed to settle. The precipitate was collected by vacuum filtration and the filter cake was washed with saturated NaHCO₃ aqueous solution followed by water. To the filtrate was added more water and the same operation was repeated as before to collect more precipitate. The filter cake was combined with that obtained from the first operation to give, after air-dried, 2-bromo-6,7,8,9-tetrahydrocyclohepta[b]indol-10(5H)-one A.323 (7.1 g, 96% yield) as an off-white solid. ¹H NMR (400 MHz, DMSO-d₆) δ ppm 11.93 (1H, br. s.), 8.28 (1H, d, J=2.6 Hz), 7.33 (1H, d, J=8.4 Hz), 7.27 (1H, dd, J=8.4, 2.6 Hz), 3.08-3.15 (2H, m), 2.63-2.70 (2H, m), 1.88-1.99 (2H, m), 1.79-1.88 (2H, m). LCMS-ESI (POS), M/Z, M+1: Found 278.0 and 280.0.

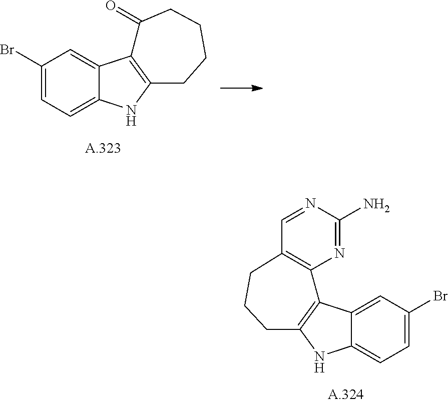

### 11-Bromo-5,6,7,8-tetrahydropyrimido[4′,′:3,4]cyclohepta[1,2-b]indol-2-amine A.324

2-Bromo-6,7,8,9-tetrahydrocyclohepta[b]indol-10(5H)-one A.323 (4.9 g, 17.6 mmol) was treated neat with t-BuOCH(NMe₂)₂ (2.0 eq., 6.14 g) at 105° C. under N₂ for 14 h. The mixture was cooled before n-PrOH (18 mL), guanidine hydrochloride (1.5 eq., 2.6 g), and MeONa (5.53 M in methanol) (1.5 eq., 5.5 mL) were added sequentially. The resulting mixture was stirred at 95° C. for 4 h and at ambient temperature for 24 h. Upon workup, the mixture was diluted with methanol and dichloromethane followed by silica gel. After concentration in vacuo, the solid mixture was dry-loaded onto silica gel column (methanol/dichloromethane) to give 11-bromo-5,6,7,8-tetrahydropyrimido[4′,′: 3 ,4]cyclohepta[1,2-b]indol-2-amine A.324 (4.2 g, 72% yield) as an off-white solid. ¹H NMR (400 MHz, DMSO-d₆) δ ppm 11.63 (1H, s), 8.88 (1H, d, J=1.8 Hz), 7.92 (1H, s), 7.19-7.30 (2H, m), 6.24 (2H, s), 3.18 (2H, t, J=6.6 Hz), 2.60-2.67 (2H, m), 1.89-1.98 (2H, m). LCMS-ESI (POS), M/Z, M+1: Found 329.0 and 331.0.

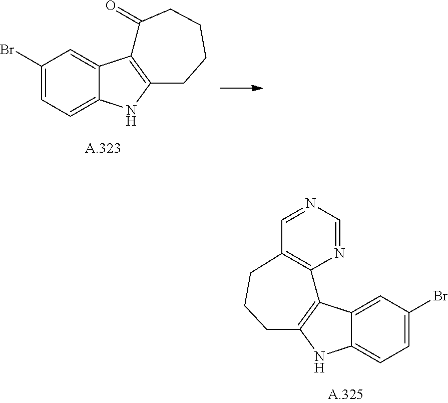

11-Bromo-5,6,7,8-tetrahydropyrimido[4′,′: 3,4]cyclohepta[1,2-b]indole A.325 was prepared using chemistry similar to that described for compound A.324 except that imidoformamide hydrochloride was used in place of guanidine hydrochloride. LCMS-ESI (POS), M/Z, M+1: Found 313.9 and 316.0.

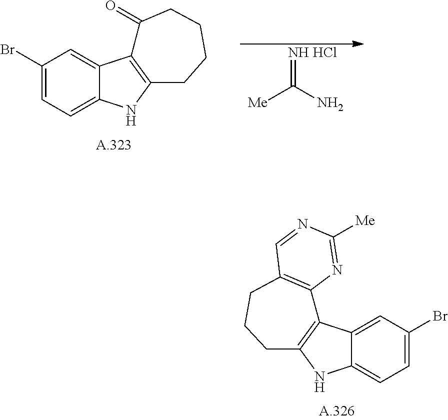

11-Bromo-2-methyl-5,6,7,8-tetrahydropyrimido[4′,5′:3,4]cyclohepta[1,2-b]indole A.326 was prepared using chemistry similar to that described for compound A.324 using acetimidamide hydrochloride in place of guanidine hydrochloride. ¹H NMR (500 MHz, DMSO-d₆) δ ppm 11.80 (1H, s), 8.91 (1H, d, J=2.0 Hz), 8.31 (1H, s), 7.23-7.34 (2H, m), 3.24 (2H, t, J=6.1 Hz), 2.78-2.84 (2H, m), 2.63 (3H, s), 1.98-2.03 (2H, m). LCMS-ESI (POS), M/Z, M+1: Found 328.0 and 330.1.

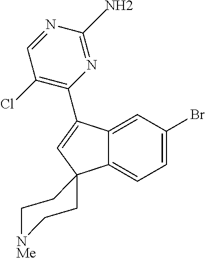

4-(5-bromo-1′-methylspiro[indene-1,4′-piperidin]-3-yl)-5-chloro-2-pyrimidinamine A.327 was prepared in the same manner as A.310 except that 2-chloro-N-(2-chloroethyl)-N-methylethanamine hydrochloride was used for the bisalkylation of A.304 and that 2 equivalents of brominating agent, i.e., NBS and NCS, were used in the allylic bromination step. LCMS-ESI (POS), M/Z, M+1: Found 405.1, 407.0 and 409.0.

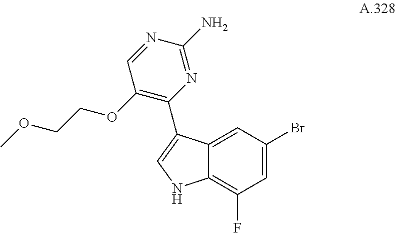

4-(5-bromo-7-fluoro-1H-indol-3-yl)-5-(2-methoxyethoxy)-2-pyrimidinamine A.328 was prepared starting with 5-bromo-7-fluoro-1H-indole using the procedures described for compound A.313.

¹H NMR (400 MHz, DMSO-d₆) δ 12.40 (s, 1H), 8.81 (d, J=1.6 Hz, 1H), 8.49 (s, 1H), 8.11 (s, 1H), 7.30 (dd, J=10.4, 1.6 Hz, 1H), 6.22 (s, 2H), 4.20-4.18 (m, 2H), 3.76-3.73 (m, 2H), 3.38 (s, 3H). LCMS-ESI (POS), M/Z, M+1: Found 381.0 and 383.0.

Compounds A.329-A.333 were prepared by the same procedures used to prepare compound A.313 beginning with compound A.311 and the appropriate alcohols as the point of diversification.

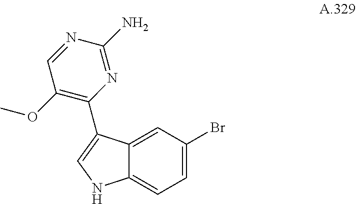

4-(5-bromo-1H-indol-3-yl)-5-methoxy-2-pyrimidinamine A.329 ¹H NMR (500 MHz, DMSO-d₆) δ 11.82 (s, 1H), 8.95 (d, J=2.0 Hz, 1H), 8.32 (d, J=2.9 Hz, 1H), 8.06 (s, 1H), 7.44 (d, J=8.6 Hz, 1H), 7.30 (dd, J=8.6, 2.0 Hz, 1H), 6.13 (s, 2H), 3.90 (s, 3H). LCMS-ESI (POS), M/Z, M+1: Found 319.0 and 321.0.

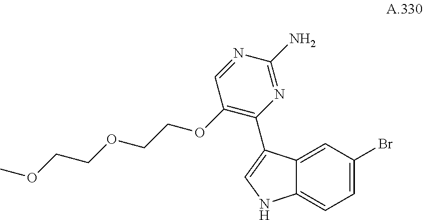

### 4-(5-bromo-1H-indol-3-yl)-5-(2-(2-methoxyethoxy)ethoxy)-2-pyrimidinamine A.330

¹H NMR (500 MHz, DMSO-d₆) δ 11.80 (s, 1H), 8.95 (d, J=2.0 Hz, 1H), 8.47 (s, 1H), 8.06 (s, 1H), 7.43 (d, J=8.5 Hz, 1H), 7.30 (dd, J=8.5, 2.0 Hz, 1H), 6.15 (s, 2H), 4.18-4.16 (m, 2H), 3.83-3.81 (m 2H), 3.64-3.62 (m, 2H), 3.53-3.51 (m, 2H), 3.27 (s, 3H). LCMS-ESI (POS), M/Z, M+1: Found 407.0 and 409.0.

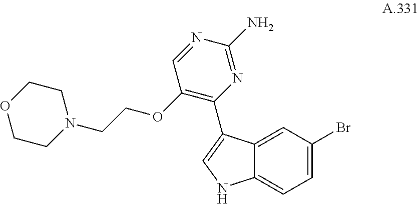

4-(5-bromo-1H-indol-3-yl)-5-(2-(4-morpholinyl)ethoxy)-2-pyrimidinamine A.331

¹H NMR (400 MHz, DMSO-d₆) δ11.89 (s, 1H), 8.97 (s, 1H), 8.73 (s, 1H), 8.09 (s, 1H), 7.44 (d, J=8.3 Hz, 1H), 7.31 (d, J=8.3 Hz, 1H), 6.14 (s, 2H), 4.16 (s, 2H), 3.65 (s, 4H), 2.76 (s, 2H), 2.52 (s, 4H). LCMS-ESI (POS), M/Z, M+1: Found 418.0 and 420.1.

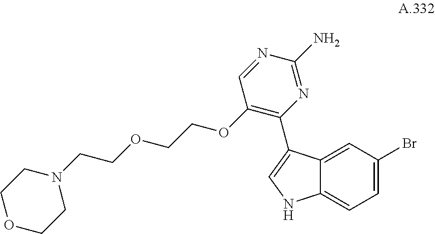

4-(5-bromo-1H-indol-3-yl)-5-(2-(2-(4-morpholinyl)ethoxy)ethoxy)-2-pyrimidinamine A.332 ¹H NMR (400 MHz, DMSO-d₆) δ 11.84 (s, 1H), 8.96 (d, J=2.0 Hz, 1H), 8.45 (s, 1H), 8.08 (s, 1H), 7.44 (d, J=8.6 Hz, 1H), 7.31 (dd, J=8.6, 2.0 Hz, 1H), 6.15 (s, 2H), 4.18 (t, J=4.4 Hz, 2H), 3.80 (t, J=4.4 Hz, 2H), 3.61 (t, J=5.9 Hz, 2H), 3.50 (t, J=4.7 Hz, 4H), 2.39 (t, J=4.7 Hz, 4H). LCMS-ESI (POS), M/Z, M+1: Found 462.0 and 464.0.

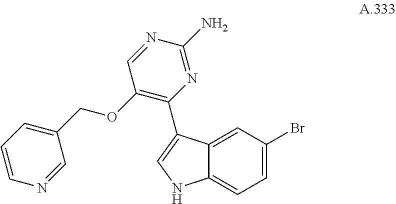

### 4-(5-bromo-1H-indol-3-yl)-5-(3-pyridinylmethoxy)-2-pyrimidinamine A.333

¹H NMR (400 MHz, DMSO-d₆) δ 11.84 (s, 1H), 8.95 (d, J=1.9 Hz, 1H), 8.63 (d, J=4.3 Hz, 1H), 8.46 (s, 1H), 8.11 (s, 1H), 7.87 (td, J=7.7, 1.9 Hz, 1H), 7.58 (d, J=7.7 Hz, 1H), 7.43-7.38 (m, 2H), 7.31 (dd, J=8.6, 2.0 Hz, 1H), 6.19 (s, 2H), 5.27 (s, 2H). LCMS-ESI (POS), M/Z, M+1: Found 396.0 and 398.0.

### General Schemes—Section B

This section illustrates procedures for making ketone building blocks for alkynyl alcohols.

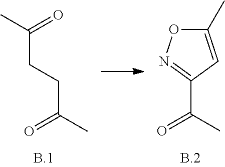

### 1-(5-methylisoxazol-3-yl)ethanone B.2

[Sauers, R. R.; Van Arnum, S. D., J. Heterocycl. Chem. 2003, 40, (4), 655-658.] 2,5-hexanedione B.1 (10 g, 87.6 mmol) was heated to 40° C., and conc. HCl (1 mL, 0.14 eq) was added. Ethylnitrite gas was generated by the dropwise addition of NaNO₂ (12.70 g, 184 mmol) in a solution of EtOH (5.6 mL) and water (50 mL) to a solution of H₂SO₄ (conc., 5.2 mL) in EtOH (5.6 mL) and water (50 mL). The ethylnitrite gas was bubbled through the acidic hexanedione solution for 2 hours while the temperature was maintained between 40-50° C. The reaction was further stirred at 40° C. for 4 hours. After cooling to room temperature, 100 mL ether was added, and the organic layer was separated, washed with sat. NaHCO₃ and brine, dried over MgSO₄ and concentrated in vacuo. Purification by flash chromatography (10% ethyl acetate in hexane) gave 1-(5-methylisoxazol-3-yl)ethanone B.2 (6.02 g). 1H NMR (500 MHz, CHLOROFORM-d) δ ppm 2.63 (3H, s), 2.48 (3H, s) Mass Spectrum (ESI) m/e=126.1 (M+1)

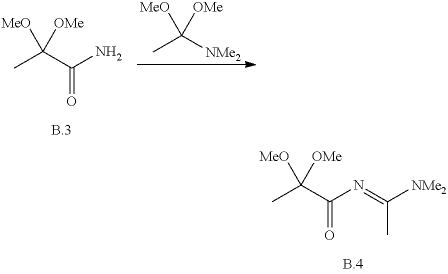

### N-(1-(dimethylamino)ethylidene)-2,2-dimethoxypropanamide B.4

[LaMattina, J. L.; Mularski, C. J., J. Org. Chem. 1984, 49, (25), 4800-4805]

A mixture of 2,2-dimethoxypropionamide B.3 (10 g, 75 mmol) and N,N-dimethylacetamide dimethylacetal (43.5 g, 326 mmol) was heated at reflux for 2 hours. The mixture was condensed in vacuo followed by distillation under reduced pressure to give N-(1-(dimethylamino)ethylidene)-2,2-dimethoxypropanamide (12.30 g) as a yellow oil. 1H NMR (500 MHz, CHLOROFORM-d) δ ppm 3.31 (6H, s), 3.10 (3H, s), 3.09 (3H, s), 2.27 (3H, s), 1.48 (3H, s) Mass Spectrum (ESI) m/e=203.2 (M+1)

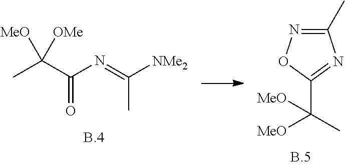

### 5-(1,1-dimethoxyethyl)-3-methyl-1,2,4-oxadiazole B.5

Hydroxylamine hydrochloride (3.8 g, 54 mmol) was dissolved in 120 mL 70% aq. acetic acid, and NaOH (2.2 g, 54 mmol) was added. To the stirring solution was added a mixture of N-(1-(dimethylamino)ethylidene)-2,2-dimethoxypropanamide B.4 in 60 mL dioxane. The reaction was heated to 100° C. for 30 minutes, cooled to room temperature and condensed in vacuo. Saturated aq. NaHCO₃ was added until the solution was slightly basic. The aqueous solution was then extracted with dichloromethane. The combined extracts were dried over MgSO₄ and evaporated to give a crude orange oil. The oil was distilled under reduced pressure to give 5-(1,1-dimethoxyethyl)-3-methyl-1,2,4-oxadiazole B.5 (6.14 g) as a clear oil. 1H NMR (500 MHz, CHLOROFORM-d) δ ppm 3.28 (6H, s), 2.39 (3H, s), 1.70 (3H, s)

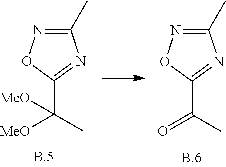

1-(3-methyl-1,2,4-oxadiazol-5-yl)ethanone B.6 5-(1,1-dimethoxyethyl)-3-methyl-1,2,4-oxadiazole B.5 (6.1 g, 35.5 mmol) and pTsOH (7.42 g, 39 mmol) were stirred in 80 mL acetone at reflux for 2 hours. The mixture was cooled to room temperature, evaporated, and the residue was tritrated with Na₂CO₃ (20% aq., 50 mL). The resulting solution was extracted with dichloromethane, and the combined extracts were dried over MgSO₄ and evaporated. The residue was distilled under reduced pressure to give 1-(3-methyl-1,2,4-oxadiazol-5-yl)ethanone B.6 (1.91 g, 43%). 1H NMR (500 MHz, CHLOROFORM-d) δ ppm 2.76 (3H, s), 2.53 (3H, s) Mass Spectrum (ESI) m/e=127.2 (M+1)

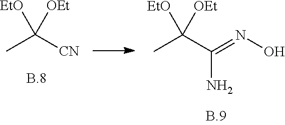

### 2,2-diethoxy-N′-hydroxypropanamidine B.9

To a stirring solution of NH₂OH—HCl (36.4 g, 524 mmol) and Na₂CO₃ (55.5 g, 524 mmol) in 150 mL water was added 2,2-diethoxypropanenitrile B.8 (10 g, 70 mmol) in 200 mL methanol, and the mixture was stirred at reflux 17 hours. The reaction was cooled to room temperature, partitioned between water and dichloromethane, and the aqueous layer was further extracted with dichloromethane. The combined organic extracts were dried over MgSO₄ and evaporated to give 2,2-diethoxy-N′-hydroxypropanamidine B.9 (3.59 g, 77%) as a white crystalline solid.

1H NMR (500 MHz, CHLOROFORM-d) δ ppm 4.87 (2H, br. s.), 3.45 (4H, q, J=7.1 Hz), 1.48 (3H, s), 1.13 (6H, t, J=7.1 Hz) Mass Spectrum (ESI) m/e=177.2 (M+1)

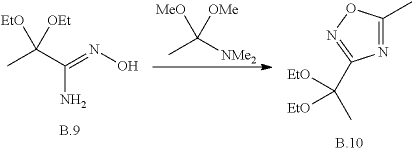

### 3-(1,1-diethoxyethyl)-5-methyl-1,2,4-oxadiazole B.10

A mixture of 2,2-diethoxy-N′-hydroxypropanamidine B.9 (1.05 g, 6 mmol) and N,N-dimethylacetamide dimethylacetal (10 mL, 75 mmol) was heated at reflux for 4 hours. The mixture was condensed in vacuo followed by flash chromatography (30-100% gradient ethyl acetate in hexane) to give 3-(1,1-diethoxyethyl)-5-methyl-1,2,4-oxadiazole B.10 (0.36 g, 66%).

1H NMR (500 MHz, CHLOROFORM-d) δ ppm 3.68 (2H, dq, J=9.1, 7.1, 7.0 Hz), 3.52 (2H, dq, J=9.2, 7.2 Hz), 2.63 (3H, s), 1.75 (3H, s), 1.27 (6H, t, J=7.0 Hz) Mass Spectrum (ESI) m/e=155.2 (M+−OEt)

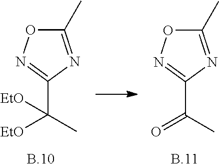

1-(5-methyl-1,2,4-oxadiazol-3-yl)ethanone B.11 This compound was prepared from B.10 in 52% yield by the procedure used for compound B.6.

1H NMR (500 MHz, CHLOROFORM-d) δ ppm 2.70 (3H, s), 2.69 (3H, s) Mass Spectrum (ESI) m/e=127.2 (M+1)

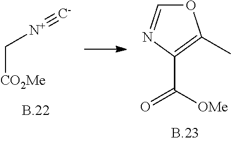

### methyl 5-methyloxazole-4-carboxylate B.23

[Suzuki M.; Iwasaki T.; Miyoshi M.; Okumura K.; K., M.; J. Org. Chem. 1973, 38, (20), 3571-3575.}

Methyl 2-isocyanoacetate B.22 (5 g, 50.5 mmol) was stirred in THF (15 mL) at room temperature as DBU (7.55 mL, 50.5 mmol) was added, followed by 4.74 mL acetic anhydride. The reaction stirred at room temperature 17 hrs. After partitioning between ethyl acetate and water, the aqueous layer was further extracted with ethyl acetate. The combined organic layers were dried over MgSO₄ and condensed. The residue was purified by flash chromatography (50-100% gradient ethyl acetate in hexane) to give methyl 5-methyloxazole-4-carboxylate B.23 (5.41 g, 76%) as a white solid. 1H NMR (500 MHz, DMSO-d₆) δ ppm 8.32 (1H, s), 3.79 (3H, s), 2.56 (3H, s) Mass Spectrum (ESI) m/e=(M+1)

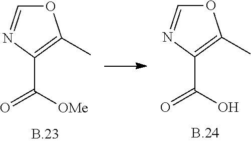

### 5-methyloxazole-4-carboxylic acid B.24

Methyl 5-methyloxazole-4-carboxylate B.23 (1 g, 7.1 mmol) was dissolved in 4 mL THF and LiOH (0.205 g, 8.5 mmol) in 12 mL water was added. After 2 hours, the THF was evaporated, and the aqueous layer was acidified with 6 N HCl and placed in the freezer to crystallize. The crystallized product was filtered and dried to give 5-methyloxazole-4-carboxylic acid. B.24 (0.69 g, 77%) 1H NMR (500 MHz, DMSO-d₆) δ ppm 12.92 (1H, br. s.), 8.29 (1H, s), 2.56 (3 H, s) Mass Spectrum (ESI) m/e=(M+1)

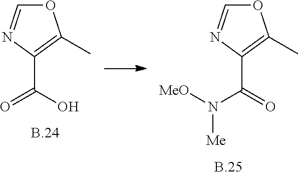

### N-methoxy-N,5-dimethyloxazole-4-carboxamide B.25

5-methyloxazole-4-carboxylic acid B.24 (490 mg, 3.9 mmol) and CDI (940 mg, 5.8 mmol) were stirred in 3 mL DMF at room temperature for 4 hours. In a separate flask, triethylamine (2.36 g, 23.4 mmol) was added to a stirring solution of N-methoxymethanamine hydrochloride (1.90 g, 19.5 mmol) in DMF (16 mL) at 0° C. To this solution was added the first mixture, and the reaction was allowed to warm to room temperature and stirred 17 hours. After being partitioned between water and ethyl acetate, the aqueous layer was further extracted with ethyl acetate, and the combined organic layers were dried over Mg SO₄ and condensed. The residue was purified by flash chromatography (1-100% gradient ethyl acetate in hexane) to give N-methoxy-N,5-dimethyloxazole-4-carboxamide B.25 (425 mg, 64%). 1H NMR (500 MHz, DMSO-d₆) δ ppm 8.31 (1H, s), 3.73 (3H, s), 3.29 (3H, s), 2.46 (3H, s) Mass Spectrum (ESI) m/e=170.9 (M+1)

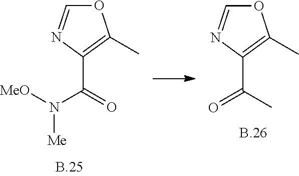

### 1-(5-methyloxazol-4-yl)ethanone B.26

To a solution of N-methoxy-N,5-dimethyloxazole-4-carboxamide B.25 (0.72 g, 4.2 mmol) in THF (30 mL) stirring at 0° C. was added methylmagnesium chloride (3.64 mL of 3 M solution in ether, 10.9 mmol). The reaction stirred at 0° C. 1 h, was quenched with methanol and acidified with 1 N HCl. The solution was diluted with dichloromethane, washed with brine, dried over MgSO₄ and condensed. Flash chromatography (1-100% gradient ethyl acetate in hexane) provided 1-(5-methyloxazol-4-yl)ethanone B.26 (106 mg, 20%). 1H NMR (500 MHz, DMSO-d₆) δ ppm 8.31 (1H, s), 3.73 (3H, s), 3.29 (3H, s), 2.46 (3H, s) Mass Spectrum (ESI) m/e=126.2 (M+1)

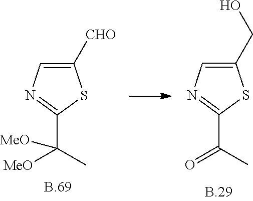

### 1-(5-(hydroxymethyl)thiazol-2-yl)ethanone B.29

2-(1,1-dimethoxyethyl)thiazole-5-carbaldehyde B.69 (1.14 g, 5.7 mmol) in 10 mL methanol was stirred at 0° C. as NaBH₄ (0.215 g, 5.7 mmol) was added portionwise. The reaction mixture stirred for 5 minutes, was quenched with sat. aq. NH₄Cl and extracted into ethyl acetate. The combined extracts were dried over MgSO₄ and condensed to give 1.18 g yellow solid. The yellow solid (0.20 g, 1 mmol) was stirred in 8 mL water-acetone (1:1 ratio) as 0.4 mL conc. HCl was added. After 2 hours, sat. aq. NaHCO₃ was added neutralize to pH 7, and the aqueous solution was extracted with dichloromethane. The combined extracts were dried over MgSO₄ and evaporated to give 1-(5-(hydroxymethyl)thiazol-2-yl)ethanone B.29 (154 mg, 98%) as a colorless crystalline solid. 1H NMR (500 MHz, CHLOROFORM-d) δ ppm 7.87 (1 H, s), 4.96 (2H, s), 2.71 (3H, s)

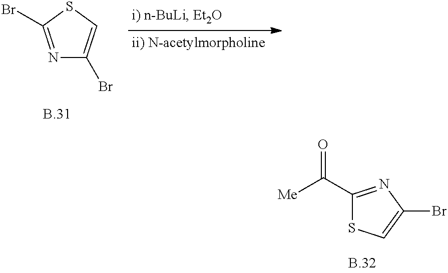

### 1-(4-bromothiazol-2-yl)ethanone B.32

[Ung, A. T.; Pyne, S. G., Tetrahedron: Asym. 1998, 9, (8), 1395-1408]

A solution of 2,4-dibromothiazole B.31 (5.0128 g, 20.63 mmol) in ether (52 mL) was cooled to −78° C. To the cooled solution was added n-BuLi (1.6 M sol. in hexane, 14.2 mL, 22.72 mmol) and the mixture was stirred at −78° C. for 30 minutes. To the cooled mixture was then added dropwise N-acetylmorpholine (3.1 mL, 26.83 mmol). The mixture was stirred at −78° C. for 1.5 hours and then room temperature for 18 hours. The mixture was diluted with ether (200 mL), washed with saturated aqueous NaHCO₃ (100 mL×1), dried over MgSO₄, filtered, and concentrated under reduced pressure. The product was purified by silica gel column chromatography using 0% to 50% gradient of ethyl acetate in hexane as eluent to give 1-(4-bromothiazol-2-yl)ethanone B.32 (3.383 g, 79.5% yield): ¹H NMR (500 MHz, CHLOROFORM-d) δ ppm 7.59 (1H, s), 2.73 (3H, s); Mass Spectrum (ESI) m/e=205.9 [M+1 (⁷⁹Br)] and 207.9 [M+1 (⁸¹Br)].

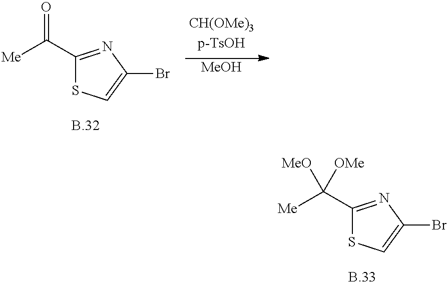

### 4-bromo-2-(1,1-dimethoxyethyl)thiazole B.33

To a solution of 1-(4-bromothiazol-2-yl)ethanone B.32 (3.383 g, 16.42 mmol) in dry methanol (55 mL) was added trimethyl orthoformate (9 mL, 82.27 mmol) and p-TsOH (2.8103 g, 14.77 mmol). The mixture was heated at reflux for 21.5 hours. The mixture was poured into saturated aqueous NaHCO₃ (100 mL). The aqueous layer was extracted with ethyl acetate (100 mL×2). The combined organic layers were washed with 1 N aqueous NaOH (100 mL×1), brine (100 mL×1), dried over MgSO₄, filtered, and concentrated under reduced pressure to give a white solid. The product was purified by silica gel column chromatography using 10% ethyl acetate in hexane as eluent to give 4-bromo-2-(1,1-dimethoxyethyl)thiazole B.33 (3.753 g, 90% yield) as a light yellow solid: ¹H NMR (400 MHz, CHLOROFORM-d) δ ppm 7.24 (1 H, s), 3.26 (6H, s), 1.73 (3H, s); Mass Spectrum (ESI) m/e=219.9 [M+1 (⁷⁹Br)] and 221.9 [M+1 (⁸¹Br)].

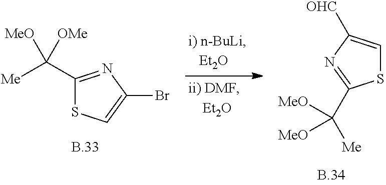

### 2-(1,1-dimethoxyethyl)thiazole-4-carbaldehyde

To a solution of 4-bromo-2-(1,1-dimethoxyethyl)thiazole B.33 (3.753 g, 14.88 mmol) in ether (55 mL) at −78° C. was added dropwise n-BuLi (1.6 M sol in hexane, 10.2 mL, 16.37 mmol) and the mixture was stirred at −78° C. After stirring at −78° C. for 30 minutes, a solution of DMF (4.6 mL, 59.53 mmol) in ether (16 mL) was added to the mixture at −78° C. and the mixture was stirred at −78° C. for 20 minutes and allowed to warm to room temperature. After stirring at room temperature for 3.5 hours, water (50 mL) was added and the aqueous mixture was extracted with ethyl acetate (50 mL×4). The combined organic layers were dried over MgSO₄, filtered, and concentrated under reduced pressure to give an orange oil. The product was purified by silica gel column chromatography using 0% to 100% gradient of ethyl acetate in hexane as eluent to give 2-(1,1-dimethoxyethyl)thiazole-4-carbaldehyde B.34 (1.87 g, 62.4% yield) as a light yellow solid: ¹H NMR (500 MHz, CHLOROFORM-d) δ ppm 10.10 (1 H, s), 8.19 (1H, s), 3.29 (6H, s), 1.78 (3H, s); Mass Spectrum (ESI) m/e=202.1 [M+1].

2-(1,1-Dimethoxyethyl)thiazole-4-carbaldehyde (B.34) was converted into 2-acetylthiazole-4-carbonitrile B.37 in three steps by the method used for the preparation of 2-acetylthiazole-5-carbonitrile B.71.

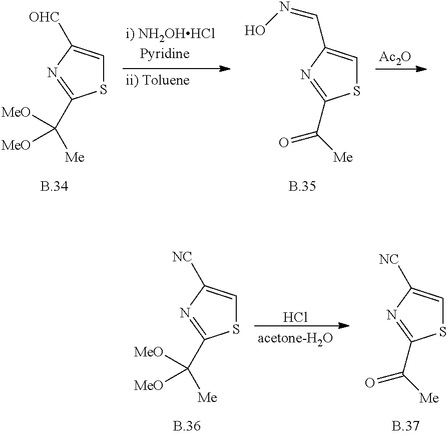

4-fluoropicolinonitrile B.40 [Kuduk, S. D.; DiPardo, R. M.; Bock, M. G., Org. Lett. 2005, 7, (4), 577-580.]

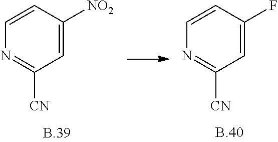

4-nitropicolinonitrile B.39 (5 g, 33.5 mmol) in 64 mL DMF was stirred at room temperature as 67 mL TBAF (1 M in THF, 67 mmol) was added. After 35 minutes, the reaction was poured into 500 mL 1:1 ethyl acetate-water. The organic layer was washed with water and brine, dried over Na₂SO₄ and condensed. The orange-brown residue was purified by flash chromatography (0-10-100% ethyl acetate in hexane) to give 4-fluoropicolinonitrile B.40 (2.78 g, 68%). 1H NMR (500 MHz, CHLOROFORM-d) δ ppm 8.72 (1H, dd, J=7.9, 5.7 Hz), 7.48 (1H, dd, J=8.1, 2.4 Hz), 7.30 (1H, ddd, J=8.1, 5.6, 2.4 Hz)

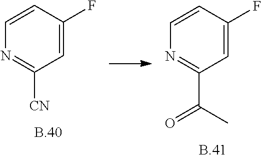

### 1-(4-fluoropyridin-2-yl)ethanone B.41

A solution of MeMgCl (3 M in THF, 22.5 mL, 67.5 mmol) was added to a stirring solution of 4-fluoropicolinonitrile B.40 (2.75 g, 22.5 mmol) in 56 mL THF at 0° C. The mixture stirred for 3 hours, gradually warming to room temperature. Sat. aq. NH₄Cl (5 mL) was added to quench the reaction, and the mixture was partitioned between ethyl acetate and water. The aqueous layer was further extracted with ethyl acetate, and the combined organic extracts were dried over Na₂SO₄ and condensed. Purification by flash chromatography (0-100% ethyl acetate in hexane) afforded 1-(4-fluoropyridin-2-yl)ethanone B.41 (1.46 g, 47%) as a yellow crystalline solid. 1H NMR (500 MHz, CHLOROFORM-d) δ ppm 8.67 (1H, dd, J=8.1, 5.4 Hz), 7.77 (1H, dd, J=9.3, 2.4 Hz), 7.21 (1H, ddd, J=8.1, 5.5, 2.6 Hz), 2.74 (3H, s)

### 1-methyl-2,3-pyrrolidinedione B.46

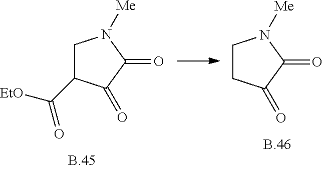

Ethyl 1-methyl-4,5-dioxo-3-pyrrolidinecarboxylate B.45 (6.76 g, 33.6 ommol) [Southwick, P. L., E. P. Previc, et al. (1956). J. Org. Chem. 21(10): 1087-1095.] was dissolved in 76 ml of acetic acid and 11 ml of 48% by weight HBr. The solution was heated to a gentle reflux for 1 hour. The reaction was then poured into 400 ml of ice and stirred until the ice melted. The aqueous layer was extracted with chloroform. The pH of the aqueous layer was adjusted to ˜7 with sat. NaHCO₃ was and then extracted with dichloromethane. The organic layer was dried over MgSO4 then concentrated under vacuum. The residue was triturated with ether and filtered to remove solids. The filtrates were concentrated under vacuum to give a reddish oil which was purified by CombiFlash chromatography eluting with acetonitrile/0.1% TFA. The fractions containing the product were concentrated under vacuum to give 1-methyl-2,3-pyrrolidinedione B.46 (214 mg,).

1H NMR (500 MHz, DMSO-d₆) δ ppm 3.56 (2H, t, J=5.4 Hz), 2.97 (3H, s), 2.62 (2H, t, J=5.5 Hz)

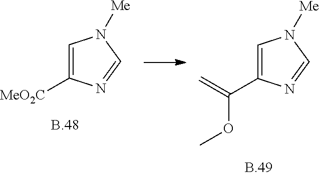

### 4-(1-methoxyethenyl)-1-methyl-1H-imidazole B.49

Methyl 1-methyl-1H-imidazole-4-carboxylate B.48 (250 mg, 1.178 mmol) was dissolved in 5 ml of anhydrous pyridine and cooled to ˜0° C. under an atmosphere of nitrogen. Tebbe Reagent (0.5M in toluene) (3.57 mL) was added slowly. The reaction was warmed to room temperature and stirred 2 hours. After cooling to 0° C., the reaction was quenched with 1 ml of 40% NaOH and 2 ml of water. The solution was diluted with 10 ml of pyridine and stirred at r.t. for 1 hour. Solvents were removed under vacuum. The residue obtained was partially dissolved in methanol and filtered. The filtrate was then purified by CombiFlash chromatography eluting with a gradient of 2% methanol/dichloromethane to 10% methanol/dichloromethane. The fractions containing the product were combined and concentrated under vacuum to give 4-(1-methoxyethenyl)-1-methyl-1H-imidazole B.49 as a brownish oil (249 mg).

ESI-MS: M+H⁺ 139.1 m/z

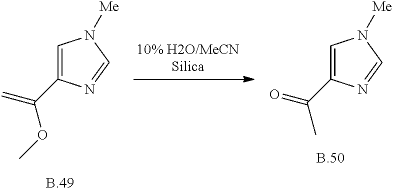

### 1-(1-methyl-1H-imidazol-4-yl)ethanone B.50

4-(1-methoxyethenyl)-1-methyl-1H-imidazole) B.49 (249 mg, 1.81 mmol), was combined with 1.9 g of silica gel in 18 ml of acetonitrile and 2 ml of water. The solution was stirred at room temperature for 20 h and then diluted with 20 ml of acetonitrile before filtering off the silica gel. The silica gel was washed with dichloromethane. The filtrate was concentrated under vacuum to give 1-(1-methyl-1H-imidazol-4-yl)ethanone B.50 (70 mg, 38%) as a white solid.

1H NMR (500 MHz, DMSO-d₆) δ ppm 7.85 (1H, s), 7.70 (1H, s), 3.70 (3H, s), 2.37 (3H, s)

### 1-(4-vinylthiophen-2-yl)ethanone B.56

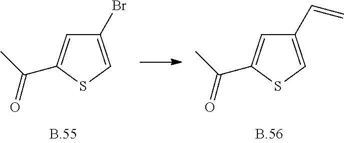

A mixture of 1-(4-bromothiophen-2-yl)ethanone B.55 (2 g, 9.75 mmol), 1,4-dioxane (32 mL), tributyl(vinyl)stannane (3.13 mL, 10.73 mmol), and Pd(PPh₃)₄ (0.5635 g, 0.49 mmol) was stirred at 100° C. for 14.5 hours. The mixture was concentrated under reduced pressure and purified by silica gel column chromatography using 0% to 40% gradient of ethyl acetate in hexane as eluent to give 1-(4-vinylthiophen-2-yl)ethanone B.56 (1.22 g, 82.2% yield) as a yellow oil: ¹H NMR (500 MHz, DMSO-d₆) δ ppm 8.18 (1H, s), 7.91 (1H, s), 6.71 (1H, dd, J=17.6, 10.8 Hz), 5.82 (1H, d, J=17.6 Hz), 5.28 (1H, d, J=11.0 Hz), 2.55 (3H, s); Mass Spectrum (ESI) m/e=153.1 [M+1].

### 1-(4-ethylthiophen-2-yl)ethanone B.57

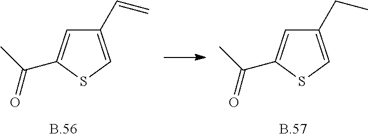

To a solution of 1-(4-vinylthiophen-2-yl)ethanone B.56 (0.674 g, 4.43 mmol) in methanol (15 mL) was added 10% Pd/C (0.4714 g, 0.443 mmol) and the mixture was stirred under H₂ gas for 1 hour. The mixture was filtered through a Celite pad and the pad was washed with methanol. The filtrate was concentrated under reduced pressure to give 1-(4-ethylthiophen-2-yl)ethanone B.57 (0.585 g, 85.7%) as a colorless oil: ¹H NMR (400 MHz, DMSO-d₆) δ ppm 7.81 (1H, s), 7.61 (1H, s), 2.61 (2H, q, J=7.4 Hz), 2.49 (3H, s), 1.19 (3H, t, J=7.6 Hz); Mass Spectrum (ESI) m/e=155.1 [M+1].

### 1-(3-methylisoxazol-5-yl)ethanone B.65

[Felman, S. W., I. Jirkovsky, et al. (1992). J. Med. Chem. 35(7): 1183-1190.]

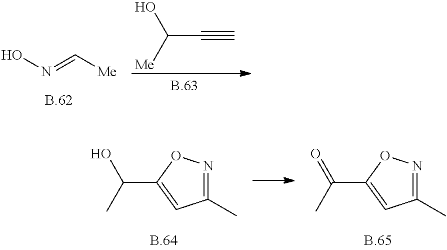

To a solution of acetaldoxime B.62 (10 g, 0.169 mmol), but-3-yn-2-ol B.63 (11.84 g, 0.169 mmol), and triethylamine (2.4 mL, 0.0169 mmol) in dichloromethane (400 mL) at 0° C. was added sodium hypochlorite solution (6% aqueous, 440 mL) over 1 hour and the mixture was allowed to warm to room temperature, and stirred at room temperature for 19 h. The organic layer was separated. The aqueous layer was extracted with dichloromethane (100 mL×2). The combined organic layers were washed with brine (200 mL×1), dried over MgSO₄, filtered, and concentrated under reduced pressure to give 1-(3-methylisoxazol-5-yl)ethanol B.64 as a yellow oil. The product was used directly for the next step. To a solution of 1-(3-methylisoxazol-5-yl)ethanol B.64 (9.54 g, 75.04 mmol) in acetone (417 mL) at 0° C. were added dropwise a solution of CrO₃ (19.06 g, 190.6 mmol) in water and 6 NH₂SO₄ (37.5 mL, 225.1 mmol) over 10 minutes. The mixture was allowed to warm to room temperature. After stirring at room temperature for 2.5 hours, saturated aqueous NaCl (100 mL) was added to the mixture. The aqueous mixture was extracted with dichloromethane (100 mL×2). The combined organic layers were washed with brine (100 mL×3), dried over MgSO₄, filtered, and concentrated under reduced pressure to give a yellow solid. The yellow solid was purified by silica gel column chromatography using 0% to 50% gradient of ethyl acetate in hexane as eluent to give 1-(3-methylisoxazol-5-yl)ethanone B.65 (2.57 g, 27.3% overall yield over two steps): ¹H NMR (400 MHz, DMSO-d₆) δ ppm 7.18 (1H, s), 2.53 (3H, s), 2.32 (3H, s); Mass Spectrum (ESI) m/e=126.2 [M+1].

### 2-(1,1-dimethoxyethyl)thiazole B.68

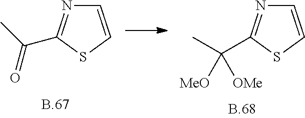

To a solution of 1-(thiazol-2-yl)ethanone B.67 (10 g, 78.63 mmol) in dry methanol (260 mL) was added trimethyl orthoformate (86 mL, 786.35 mmol) and p-TsOH (13.462 g, 70.77 mmol). The mixture was heated at reflux for 23 hours. The mixture was poured into saturated aqueous NaHCO₃ (500 mL). The aqueous layer was extracted with ethyl acetate (200 mL×4). The combined organic layers were washed with 1 N aqueous NaOH (300 mL), brine (300 mL×1), dried over Na₂SO₄, filtered, and concentrated under reduced pressure to give a yellow oil. The product was purified by silica gel column chromatography using 25% ethyl acetate in hexane as eluent to give 2-(1,1-dimethoxyethyl)thiazole B.68 (7.91 g, 58.1% yield) as a light yellow oil: ¹H NMR (400 MHz, CHLOROFORM-d) δ ppm 7.83 (1H, d, J=3.1 Hz), 7.32 (1H, d, J=3.1 Hz), 3.26 (6H, s), 1.74 (3H, s); Mass Spectrum (ESI) m/e=174.1 [M+1].

### 2-(1,1-dimethoxyethyl)thiazole-5-carbaldehyde B.69

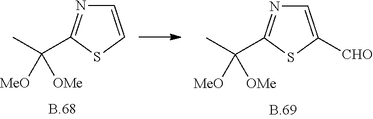

n-BuLi (1.6 M sol in hexane, 14.4 mL, 23.04 mmol) was added dropwise to a solution of 2-(1,1-dimethoxyethyl)thiazole B.68 (4 g, 23.09 mmol) in THF (50 mL) at −78° C. After stirring at −78° C. for 20 minutes, a solution of DMF (3.7 mL, 47.54 mmol) in THF (19 mL) was added and the mixture was stirred at −78° C. for 40 minutes. The reaction was allowed to warm to room temperature and stirred for 16 hours. Saturated aqueous NH₄Cl solution (50 mL) was added and the aqueous mixture was extracted with dichloromethane (100 mL×4). The combined organic layers were washed with brine (100 mL×1), dried over MgSO₄, filtered, and concentrated under reduced pressure to give a dark red oil. The product was purified by silica gel column chromatography using 0% to 70% gradient of ethyl acetate in hexane as eluent to give 2-(1,1-dimethoxyethyl)thiazole-5-carbaldehyde B.69 (2.27 g, 48.9% yield) as a yellow solid: ¹H NMR (500 MHz, CHLOROFORM-d) δ ppm 10.04 (1H, s), 8.42 (1H, s), 3.27 (6H, s), 1.74 (3H, s); Mass Spectrum (ESI) m/e=202.0 [M+1].

### 2-acetylthiazole-5-carbaldehyde oxime B.70

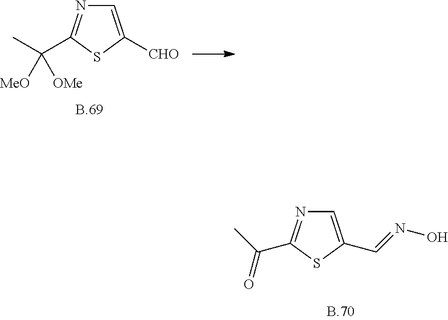

To a mixture of NH₂OH.HCl (0.38 g, 5.47 mmol) and pyridine (0.9 mL) was added 2-(1,1-dimethoxyethyl)thiazole-5-carbaldehyde B.69 (1.1 g, 5.47 mmol) in installments over 1 minute. The mixture was stirred at room temperature for 20 minutes. Toluene (13 mL) was then added and the mixture was heated at reflux using a Dean-Stark water trap for 2 hours. The mixture was poured into ice water (100 mL) and extracted with ethyl acetate (100 mL×2). The combined organic layers were washed with brine (100 mL×1), dried over Na₂SO₄, filtered, and concentrated under reduced pressure to give 2-acetylthiazole-5-carbaldehyde oxime B.70 (0.551 g, 59.2% yield) as an orange solid: ¹H NMR (500 MHz, DMSO-d₆) δ ppm 12.77 (1H, s), 8.44 (1H, s), 8.14 (1H, s), 2.65 (3H, s); Mass Spectrum (ESI) m/e=171.1 [M+1].

### 2-acetylthiazole-5-carbonitrile B.71

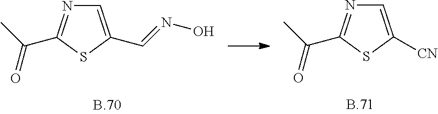

A suspension of 2-acetylthiazole-5-carbaldehyde oxime B.70 (0.551 g, 3.24 mmol) in acetic anhydride (3 mL) was heated at reflux for 4 hours. The mixture was concentrated under reduced pressure and then treated with cold 2 N aqueous Na₂CO₃ (50 mL). The aqueous mixture was extracted with dichloromethane (50 mL×3). The combined organic layers were dried over Na₂SO₄, filtered, and concentrated under reduced pressure to give a dark syrup. The product was purified by silica gel column chromatography using 0% to 50% gradient of ethyl acetate in hexane as eluent to give 2-acetylthiazole-5-carbonitrile B.71 (0.268 g, 54.3% yield) as a light yellow solid: ¹H NMR (500 MHz, CHLOROFORM-d) δ ppm 8.40 (1H, s), 2.76 (3 H, s); Mass Spectrum (ESI) m/e=152.9 [M+1].

### (2-(1,1-dimethoxyethyl)thiazol-4-yl)methanol B.76

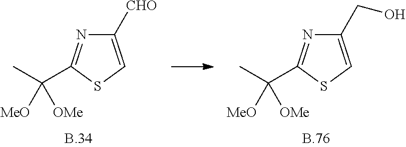

To a solution of 2-(1,1-dimethoxyethyl)thiazole-4-carbaldehyde B.34 (0.369 g, 1.83 mmol) in methanol (3.5 mL) at 0° C. was added NaBH₄ (0.0694 g, 1.83 mmol) and the mixture was stirred for 1 hour. To the mixture was added saturated aqueous NH₄Cl (20 mL). The mixture was extracted with dichloromethane (50 mL×3). The combined organic layers were dried over MgSO₄, filtered, and concentrated under reduced pressure to give (2-(1,1-dimethoxyethyl)thiazol-4-yl)methanol B.76 (0.36 g, 96.6% yield) as a white solid: ¹H NMR (400 MHz, CHLOROFORM-d) δ ppm 7.20 (1H, s), 4.80 (2H, s), 3.26 (6H, s), 1.73 (3H, s); Mass Spectrum (ESI) m/e=204.1 [M+1].

### 1-(4-(hydroxymethyl)thiazol-2-yl)ethanone

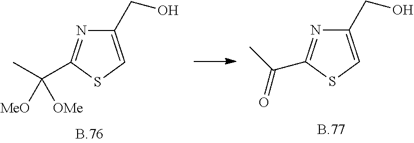

Conc. HCl (0.066 mL, 7.98 mmol) was added dropwise to a solution of (2-(1,1-dimethoxyethyl)thiazol-4-yl)methanol B.76 (0.36 g, 1.77 mmol) in acetone-water (1:1, 13.6 mL) and the mixture was stirred at room temperature for 1 hour. The acetone was removed under reduced pressure. The mixture was diluted with dichloromethane (50 mL) and washed with saturated aqueous NaHCO₃ (25 mL×2). The aqueous mixture was extracted with dichloromethane (50 mL×1). The combined organic layers were washed with brine (50 mL×1), dried over Na₂SO₄, filtered, and concentrated under reduced pressure to give colorless syrup. The product was purified by silica gel column chromatography using 0% to 100% gradient of ethyl acetate in hexane to give 1-(4-(hydroxymethyl)thiazol-2-yl)ethanone B.77 (0.214 g, 77% yield): Mass Spectrum (ESI) m/e=158.1 [M+1].

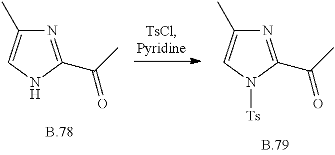

1-(4-Methyl-1-tosyl-1H-imidazol-2-yl)ethanone (B.79). p-Toluenesulfonyl chloride (1.275 g, 6.69 mmol) was added to a cold solution of 1-(4-methyl-1H-imidazol-2-yl)ethanone B.78 (691.7 mg, 5.57 mmol) [Bell, A. S., S. F. Campbell, et al. (1989). J. Med. Chem. 32(7): 1552-1558] in dry pyridine (10.00 mL). The mixture was allowed to warm to room temperature then heated to 60° C. Upon completion, the reaction was cooled, diluted with water and extracted three times with ethyl acetate. The ethyl acetate extracts were combined and washed once with brine, then dried over anhydrous sodium sulfate. After filtration and concentration, the residue was used without purification.

### General Schemes—Section C

This section illustrates procedures for making alkynyl alcohol building blocks.

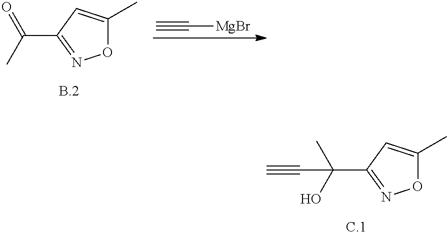

2-(5-methylisoxazol-3-yl)but-3-yn-2-ol C.1 To a stirring solution of ethynylmagnesium bromide (0.5 M in THF, 144 mL, 72 mmol) at 0° C. was added 1-(5-methylisoxazol-3-yl)ethanone (6.0 g, 48 mmol) B.2 in THF (96 mL) slowly. The reaction was allowed to warm to room temperature and stirred for 3 hours. After being quenched with conc. aq. NH₄Cl (150 mL), the THF was removed in vacuo and the aqueous residue was extracted with ethyl acetate (100 mL×2). The combined organic extracts were dried over MgSO₄, filtered, and concentrated under reduced pressure to give a dark brown oil. The dark brown oil was purified by silica gel column chromatography using 10% of ethyl acetate in hexane as eluent to give 2-(5-methylisoxazol-3-yl)but-3-yn-2-ol C.1 (4.16 g, 57% yield): ¹H NMR (500 MHz, CHLOROFORM4) δ ppm 3.15 (1H, br. s.), 2.63 (1H, s), 2.42 (3H, s), 1.86 (3H, s); Mass Spectrum (ESI) m/e=152.1 [M+1].

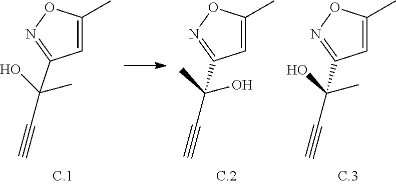

### (R)-2-(5-methylisoxazol-3-yl)but-3-yn-2-ol C.2 and (S)-2-(5-methylisoxazol-3-yl)but-3-yn-2-ol C.3

The racemic mixture C.1 was separated on preparative chiral column (Chiralcel OD-H column) using isopropanol and hexane. The stereochemistry of each enantiomer was confirmed after conversion of C.2 to 14 in Example 14 and C.3 to 15 in Example 15. Based on the biochemical potency of 14 and 15, compared to examples prepared from component C.5 and C.6, the stereochemistry was confirmed as the first eluted enantiomer C.2 as R-isomer and the second eluted enantiomer C.3 as S-isomer.

(R)-2-(5-methylisoxazol-3-yl)but-3-yn-2-ol C.2: Mass Spectrum (ESI) m/e=152.1 (M+1); [α]^{D}=−8.6. (c=0.14 in methanol)

(S)-2-(5-methylisoxazol-3-yl)but-3-yn-2-ol C.3: Mass Spectrum (ESI) m/e=152.1 (M+1); [α]^{D}=+11.6 (c=0.19 in methanol)

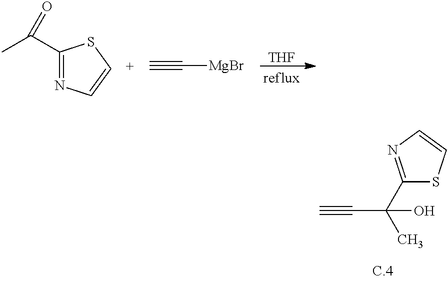

2-(Thiazol-2-yl)but-3-yn-2-ol C.4: To a solution of 0.5 M ethynylmagnesium bromide (126 mL, 63 mmol) was added solution of 2-acetylthiazole (7.72 g, 60 mmol) in THF (60 mL) at room temperature then the mixture was refluxed for 30 min. The mixture was poured into iced 2N HCl (36 mL) and extracted with ethyl ether. The organic layers were washed with brine, dried, and concentrated. The residue was washed with dichloromethane/hexane co-solvent to give the titled compound C.4 as white solid (8.45 g, 92%). ¹H NMR (400 MHz, CDCl₃) δ 8.02 (1H, d, J=3.0 Hz), 7.69 (1H, d, J=3.0 Hz), 2.76 (3H, s) ppm; LCMS-ESI (POS), M/Z, M+1: Found 154.1, Calculated 154.1.

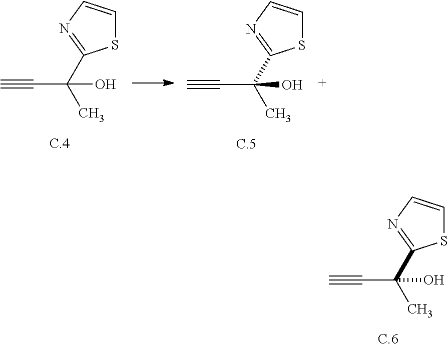

(R)-2-(Thiazol-2-yl)but-3-yn-2-ol: Racemic 2-(thiazol-2-yl)but-3yn-2-ol was resolved using chiral-HPLC (column: chiralcel-OD, 5 cm×50 cm, 20μ) eluting with 7% iso-propanol in hexane (flow rate: 100 mL/min). The first eluting compound is (S)-2-(thiazol-2-yl)but-3-yn-2-ol C.5: [α]^{D}=+37.6 in methanol. Its absolute structure was determined by crystal-structure X-ray analysis.

The second eluting compound is (R)-2-(thiazol-2-yl)but-3-yn-2-ol C.6: [α]^{D}=−30.4; −34.3 in methanol; ¹H NMR (400 MHz, CDCl₃) δ 8.02 (1H, d, J=3.0 Hz), 7.69 (1H, d, J=3.0 Hz), 2.76 (3H, s) ppm; LCMS-ESI (POS), M/Z, M+1: Found 154.1, Calculated 154.1.

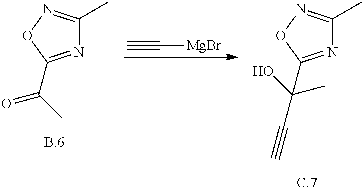

2-(3-methyl-1,2,4-oxadiazol-5-yl)but-3-yn-2-ol C.7 was prepared from B.6 in 42% yield by the procedure used for compound C.1.

1H NMR (500 MHz, CHLOROFORM-d) δ ppm 3.87 (1H, br. s.), 2.71 (1H, s), 2.44 (3H, s), 1.96 (3H, s) Mass Spectrum (ESI) m/e=153.1 (M+1)

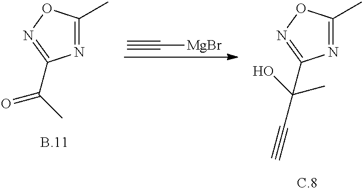

2-(5-methyl-1,2,4-oxadiazol-3-yl)but-3-yn-2-ol C.8 This compound was prepared from B.11 in 72% yield by the procedure used for compound C.1.

1H NMR (400 MHz, CHLOROFORM-d) δ ppm 3.02 (1H, br. s.), 2.68 (1H, s), 2.64 (3H, s), 1.94 (3H, s) Mass Spectrum (ESI) m/e=153.1 (M+1)

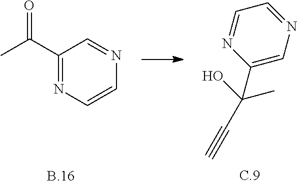

2-(pyrazin-2-yl)but-3-yn-2-ol C.9. This compound was prepared from 1-(pyrazin-2-yl)ethanone B.16 in 81% yield by the procedure used for compound C.1.

1H NMR (500 MHz, CHLOROFORM-d) δ ppm 8.98 (1H, d, J=1.2 Hz), 8.55 (2H, d, J=18.6 Hz), 4.24 (1H, s), 2.65 (1H, s), 1.86 (3H, s) Mass Spectrum (ESI) m/e=149.1 (M+1)

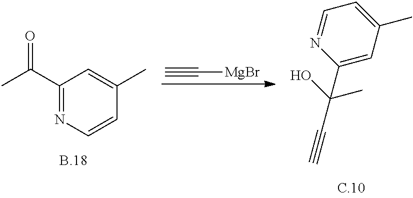

2-(4-methylpyridin-2-yl)but-3-yn-2-ol C.10. This compound was prepared from 1-(4-methylpyridin-2-yl)ethanone B.18 in 35% yield by the procedure used for compound C.1.

1H NMR (500 MHz, CHLOROFORM-d) δ ppm 8.38 (1H, d, J=5.1 Hz), 7.42 (1H, s), 7.09 (1 H, d, J=5.1 Hz), 5.57 (1H, s), 2.54 (1H, s), 2.42 (3H, s), 1.78 (3H, s) Mass Spectrum (ESI) m/e=162.1 (M+1)

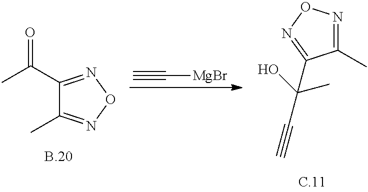

2-(4-methyl-1,2,5-oxadiazol-3-yl)but-3-yn-2-ol C.11. This compound was prepared from 1-(4-methyl-1,2,5-oxadiazol-3-yl)ethanone B.20 in 94% yield by the procedure used for compound C.1 1H NMR (400 MHz, CHLOROFORM-d₆) δ ppm 2.72 (1H, s), 2.56 (3H, s), 2.02 (3H, s) Mass Spectrum (ESI) m/e=153.1 (M+1)

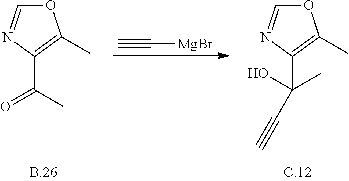

2-(5-methyloxazol-4-yl)but-3-yn-2-ol C.12 This compound was prepared from B.26 in 45% yield by the procedure used for compound C.1. 1H NMR Mass Spectrum (ESI) m/e=152.2 (M+1)

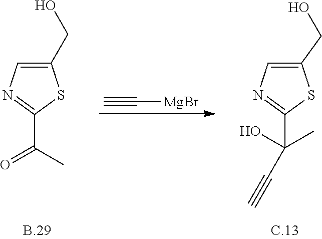

### 2-(5-(hydroxymethyl)thiazol-2-yl)but-3-yn-2-ol C.13

This compound was prepared from B.29 in 44% yield by the procedure used for compound C.1.

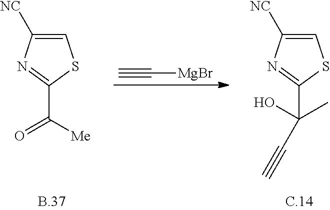

### 2-(2-hydroxybut-3-yn-2-yl)thiazole-4-carbonitrile C.14

This compound was prepared from B.37 in 10.5% yield by the procedure used for compound C.1. 1H NMR (500 MHz, CHLOROFORM-d) δ ppm 8.00 (1H, s), 3.24 (1H, s), 2.76 (1H, s), 1.98 (3H, s)

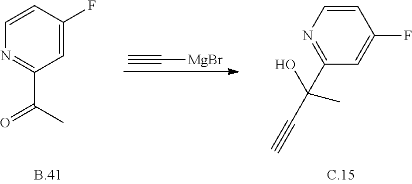

### 2-(4-fluoropyridin-2-yl)but-3-yn-2-ol C.15

This compound was prepared from B.41 in 61.5% yield by the procedure used for compound C.1. 1H NMR (500 MHz, CHLOROFORM-d) δ ppm 8.53 (1H, dd, J=8.1, 5.6 Hz), 7.36 (1H, dd, J=9.3, 2.4 Hz), 7.03 (1H, ddd, J=7.9, 5.6, 2.1 Hz), 5.07 (1H, s), 2.59 (1H, s), 1.80 (3H, s)

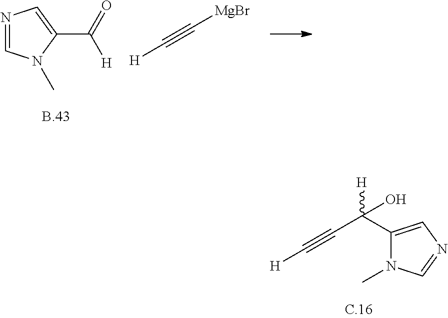

### 1-(1-methyl-1H-imidazol-5-yl)-2-propyn-1-ol C.16

This compound was prepared from B.43 by the procedure used for compound C.1 ¹H NMR (500 MHz, DMSO-d6) δ 7.56 (br s, 1H), 6.88 (br s, 1H), 6.05 (d, J=6.1 Hz, 1H), 5.48 (dd, J=5.9 Hz, J=2.2 Hz, 1H), 3.66 (s, 3H), 3.53 (br d, J=2.2 Hz, 1H); ESI MS: M+H⁺ 137.1 m/z

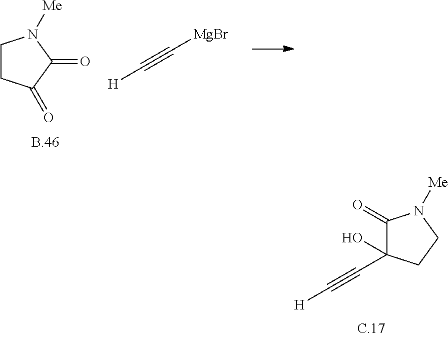

3-ethynyl-3-hydroxy-1-methyl-2-pyrrolidinone C.17 was synthesized from B.46 by the procedure used to prepare compound C.1; the title compound was obtained as a brownish oil (83 mg). ESI-MS: M+ H⁺ 140.1 m/z

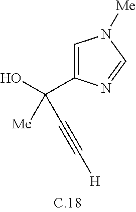

2-(1-methyl-1H-imidazol-4-yl)-3-Butyn-2-ol C.18 was synthesized from B.50 by the procedure used to prepare C.1; the title compound was obtained as a brownish oil (66 mg).

ESI-MS: M+H⁺ 151.0 m/z

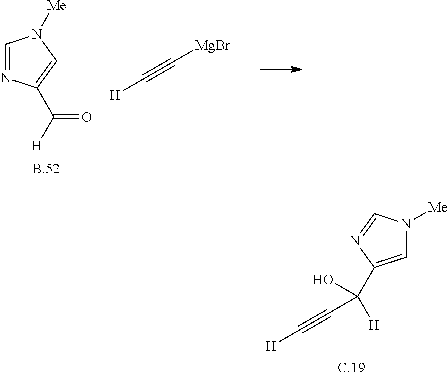

### 1-(1-methyl-1H-imidazol-4-yl)prop-2-yn-1-ol C.19

1-methyl-1H-imidazole-4-carbaldehyde B.52 was used to prepare C.19 by the procedure used to prepare C.1; the title compound was obtained as a yellow oil (654 mg).

ESI-MS: M+H⁺ 137.2 m/z

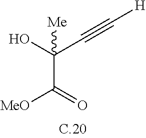

methyl 2-hydroxy-2-methyl-3-butynoate C.20 was synthesized as a yellowish oil (1.78 g) from methyl pyruvate by the procedure used to prepare C.1. 1H NMR (400 MHz, DMSO-d₆) δ ppm 6.31 (1H, s), 3.70 (3H, s), 3.47 (1H, s), 1.52 (3H, s)

### 2-(4-ethylthiophen-2-yl)but-3-yn-2-ol

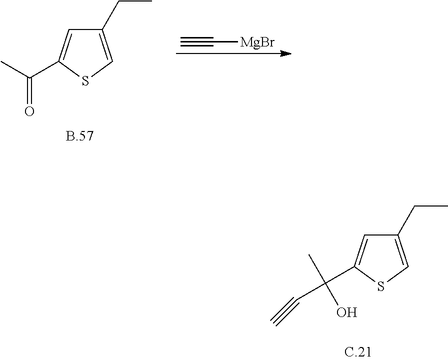

To a solution of 1-(4-ethylthiophen-2-yl)ethanone B.57 (0.58 g, 3.76 mmol) in THF was added ethynylmagnesium bromide (0.5 M sol. in THF, 8.3 mL, 4.14 mmol) and the mixture was stirred at 60° C. for 30 minutes. The mixture was poured into cold 2 N aqueous HCl. The aqueous mixture was extracted with ether (50 mL×2). The combined organic layers were dried over MgSO₄, filtered, and concentrated under reduced pressure to give a brown solid. The product was purified by silica gel column chromatography using 0% to 30% gradient of ethyl acetate in hexane as eluent to give 2-(4-ethylthiophen-2-yl)but-3-yn-2-ol C.21 (0.43 g, 63.4% yield) as a yellow oil: ¹H NMR (500 MHz, DMSO-d₆) δ ppm 6.99 (1H, d, J=1.0 Hz), 6.94 (1H, d, J=1.2 Hz), 6.31 (1H, s), 3.52 (1H, s), 2.51-2.58 (2H, m), 1.69 (3H, s), 1.15 (3 H, t, J=7.6 Hz).

### 2-cyclobutylbut-3-yn-2-ol

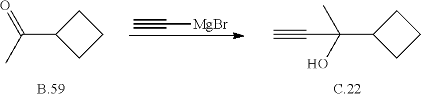

To a stirring solution of ethynylmagnesium bromide (0.5 M in THF, 61.1 mL, 30.57 mmol) in THF (40.8 mL) at 0° C. was added dropwise 1-cyclobutylethanone B.59 (2.2 mL, 20.38 mmol). The reaction was allowed to warm to room temperature and stirred for 2 hours. After being quenched with conc. aq. NH₄Cl (100 mL), the THF was removed in vacuo and the aqueous residue was extracted with ethyl acetate (100 mL×2). The combined organic extracts were dried over MgSO₄, filtered, and concentrated under reduced pressure to give 2-cyclobutylbut-3-yn-2-ol C.22 (1.57 g, 62% yield) as an orange oil: ¹H NMR (400 MHz, DMSO-d₆) δ ppm 5.12 (1H, s), 3.19 (1H, s), 2.33-2.43 (1H, m), 1.57-2.04 (6H, m), 1.19 (3 H, s).

### 2-(1-methyl-1H-pyrazol-3-yl)but-3-yn-2-ol

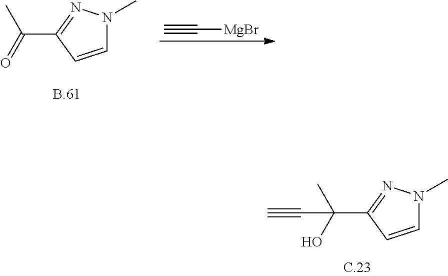

To a stirring solution of ethynylmagnesium bromide (0.5 M in THF, 121 mL, 60.4 mmol) at 0° C. was added 1-(1-methyl-1H-pyrazol-3-yl)ethanone C.23 (5 g, 40.28 mmol) in THF (80 mL) slowly. The reaction was allowed to warm to room temperature and stirred for 4 hours. After being quenched with conc. aq. NH₄Cl (200 mL), the THF was removed in vacuo and the aqueous residue was extracted with ethyl acetate (200 mL×2). The combined organic extracts were dried over MgSO₄, filtered, and concentrated under reduced pressure to give a yellow oil. The yellow oil was purified by silica gel column chromatography using 0% to 50% gradient of dichloromethane-methanol-NH₄OH (89:9:1) in dichloromethane as eluent to give 2-(1-methyl-1H-pyrazol-3-yl)but-3-yn-2-ol C.23 (4.52 g, 74.7% yield): ¹H NMR (400 MHz, DMSO-d₆) δ ppm 7.55 (1H, d, J=2.0 Hz), 6.22 (1H, d, J=2.3 Hz), 5.78 (1H, s), 3.78 (3H, s), 3.28 (1H, s), 1.65 (3H, s); Mass Spectrum (ESI) m/e=151.1 [M+1].

### 2-(3-methylisoxazol-5-yl)but-3-yn-2-ol C.24

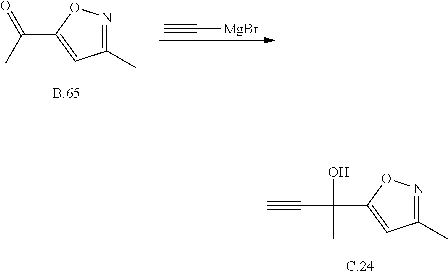

To a stirring solution of ethynylmagnesium bromide (0.5 M in THF, 62 mL, 30.76 mmol) at 0° C. was added 1-(3-methylisoxazol-5-yl)ethanone B.65 (2.57 g, 20.5 mmol) in THF (41 mL) slowly. The reaction was allowed to warm to room temperature and stirred for 5 hours. After being quenched with conc. aq. NH₄Cl (100 mL), the THF was removed in vacuo and the aqueous residue was extracted with ethyl acetate (100 mL×2). The combined organic extracts were dried over MgSO₄, filtered, and concentrated under reduced pressure to give 2-(3-methylisoxazol-5-yl)but-3-yn-2-ol C.24 (3.2 g) as a brown oil: ¹H NMR (400 MHz, DMSO-d₆) δ ppm 6.56 (1H, s), 6.27 (1H, s), 3.59 (1H, s), 2.21 (3H, s), 1.68 (3H, s); Mass Spectrum (ESI) m/e=152.2 [M+1].

### 2-(2-hydroxybut-3-yn-2-yl)thiazole-5-carbonitrile C.25

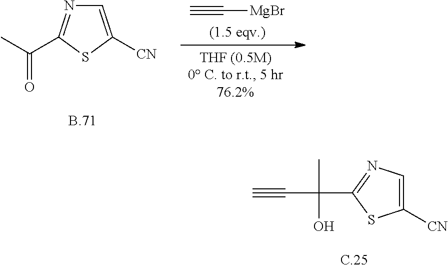

2-acetylthiazole-5-carbonitrile B.71 (0.262 g, 1.72 mmol) in THF (3.4 mL) was added slowly to a stirring solution of ethynylmagnesium bromide (0.5 M in THF, 5.2 mL, 2.58 mmol) at 0° C. The reaction was allowed to warm to room temperature and stirred for 5 hours. After quenching with conc. aq. NH₄Cl (20 mL), the organic solvent was removed in vacuo and the aqueous residue was extracted with ethyl acetate (50 mL×3). The combined organic extracts were dried over MgSO₄, filtered, and concentrated under reduced pressure to give a dark syrup. The dark syrup was purified by silica gel column chromatography using 0% to 50% gradient of ethyl acetate in hexane as eluent to give 2-(2-hydroxybut-3-yn-2-yl)thiazole-5-carbonitrile

C.25 (0.234 g, 76.2% yield) as a yellow syrup: ¹H NMR (500 MHz, DMSO-d₆) δ ppm 8.53-8.67 (1H, m), 7.38-7.53 (1H, m), 3.61-3.77 (1H, m), 1.70-1.83 (3H, m); Mass Spectrum (ESI) m/e=179.1 [M+1].

### 2-(4-(hydroxymethyl)thiazol-2-yl)but-3-yn-2-ol

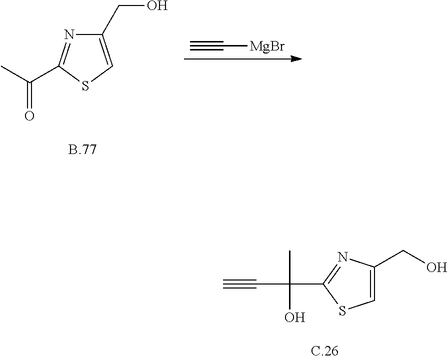

1-(4-(hydroxymethyl)thiazol-2-yl)ethanone B.77 (0.214 g, 1.36 mmol) in THF (2.7 mL) was added slowly to a stirring solution of ethynylmagnesium bromide (0.5 M in THF, 6.8 mL, 3.4 mmol) at 0° C. The reaction was allowed to warm to room temperature and stirred for 6 hours. After quenching with conc. aq. NH₄Cl (20 mL), the THF was removed in vacuo and the aqueous residue was extracted with ethyl acetate (50 mL×2). The combined organic extracts were dried over MgSO₄, filtered, and concentrated under reduced pressure to give an orange oil. The orange oil was purified by silica gel column chromatography using 0% to 100% gradient of ethyl acetate in hexane as eluent to give 2-(4-(hydroxymethyl)thiazol-2-yl)but-3-yn-2-ol C.26 (0.0794 g, 31.8% yield) as a white solid: Mass Spectrum (ESI) m/e=184.0 [M+1].

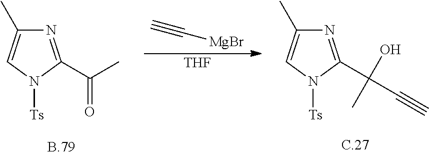

2-(4-Methyl-1-((4-methylphenyl)sulfonyl)-1H-imidazol-2-yl)-3-butyn-2-ol (C.27). To a dry flask containing dry THF (10.00 mL) was added 0.5 M solution of ethynylmagnesium bromide in THF (14.5 mL, 7.25 mmol) then cooled to 0° C. After 10 minutes, a pre-mixed solution of 1-(4-methyl-1-tosyl-1H-imidazol-2-yl)ethanone (777.7 mg, 2.79 mmol) in dry THF (5.00 mL) was added dropwise. Upon complete addition, the mixture was stirred at room temperature. After 4 hours, the reaction was quenched with saturated aq. ammonium chloride solution then extracted three times with ethyl acetate. After concentration, the residue was purified by silica gel flash chromatography (0-50% ethyl acetate in hexanes to afford 2-(4-methyl-1-((4-methylphenyl)sulfonyl)-1H-imidazol-2-yl)-3-butyn-2-ol (C.27) (0.72 g, 85%). ¹H NMR (500 MHz, DMSO-d₆) δ ppm 7.92 (2H, d, J=8.6 Hz), 7.52 (1H, d, J=1.2 Hz), 7.43 (2H, d, J=8.3 Hz), 6.16 (1H, s), 3.47 (1H, s), 2.40 (3H, s), 2.07 (3H, s), 1.80 (3H, s).

Additional alkynyl alcohols disclosure C.28-C.69 were either commercially available or prepared as in the literature or in similar fashion from the corresponding ketones.

C.28 1-ethynylcyclobutanol U.S. Pat. No. 5,583,227, 1996

C.29 2-(thiazol-4-yl)but-3-yn-2-ol (ketone precursor [38205-66-2])

C.30 2-(1-tosyl-1H-pyrazol-5-yl)but-3-yn-2-ol (derived from ketone [175277-40-4])

C.34 2-cyclopentylbut-3-yn-2-ol U.S. Pat. No. 4,608,388 , 1986

C.35 2-methylbut-3-yn-2-ol [115-19-5]

C.36 1-ethynylcyclopentanol [17356-19-3]

C.37 1-ethynylcyclohexanol [978-27-3]

C.38 benzyl 2,2-dimethylbut-3-ynoate [204588-77-2]

C.40 4-ethynyltetrahydro-2H-pyran-4-ol U.S. Pat. No. 6,130,239 , 2000

C.42 2-(thiophen-2-yl)but-3-yn-2-ol (Vaitiekunas, A. and F. F. Nord (1954). J. Amer. Chem. Soc. 76: 2733-2735)

C.43 3-ethynyltetrahydrothiophen-3-ol (Karrer, P. and A. Kieso (1944). Helv. Chim. Acta, 27: 1285.)

C.44 3-methylpent-1-yn-3-ol [77-75-8]

C.45 3,4-dimethylpent-1-yn-3-ol [1482-15-1]

C.46 3,4,4-trimethylpent-1-yn-3-ol [993-53-3]

C.47 2-(pyridin-4-yl)but-3-yn-2-ol (ketone precursor [1122-54-9])

C.48 2-(pyridin-3-yl)but-3-yn-2-ol (ketone precursor [350-03-8])

C.49 2-(pyridin-2-yl)but-3-yn-2-ol (Wrobel, D. (1964). Chemistry and Industry 1758)

C.50 2-phenylbut-3-yn-2-ol [127-66-2]

C.51 1,1,1-trifluoro-2-phenylbut-3-yn-2-ol [99727-20-5]

C.52 1,1,1-trifluoro-2-(3-fluorophenyl)but-3-yn-2-ol (ketone precursor [708-64-5])

C.53 1,1,1-trifluoro-2-methylbut-3-yn-2-ol (Tarrant P., Warner D. A., et al. (1953). J. Amer. Chem. Soc. 75(17): 4360-4362.)

C.54 2-(furan-2-yl)but-3-yn-2-ol (Semmelhack, M. F. and N. Jeong (1990). Tetrahedron Lett., 31(5): 605-608.)

C.55 2-(5-methylfuran-2-yl)but-3-yn-2-ol (ketone precursor [1193-79-9])

C.56 2-(2,5-dimethylfuran-3-yl)but-3-yn-2-ol (ketone precursor [10599-70-9])

C.57 2-(oxazol-2-yl)but-3-yn-2-ol (ketone precursor [77311-07-0])

C.58 2-(5-methyloxazol-2-yl)but-3-yn-2-ol (ketone precursor Sauers, R. R. and S. D. Van Arnum (1987). Tetrahedron Lett. 28(47): 5797-5800)

C.59 2-(thiophen-3-yl)but-3-yn-2-ol (ketone precursor [1468-83-3])

C.60 2-(4-methylthiophen-2-yl)but-3-yn-2-ol (ketone precursor [13679-73-7])

C.61 2-(3-methylthiophen-2-yl)but-3-yn-2-ol (ketone precursor [13679-72-6])

C.62 2-(5-methylthiophen-2-yl)but-3-yn-2-ol (ketone precursor [13679-74-8])

C.63 2-(5-chlorothiophen-2-yl)but-3-yn-2-ol (Vaitiekunas, A. and F. F. Nord (1954). J. Amer. Chem. Soc. 76: 2733-2735)

C.64 2-(3-chlorothiophen-2-yl)but-3-yn-2-ol (ketone precursor [89581-82-8])

C.65 2-(5-methylthiazol-2-yl)but-3-yn-2-ol (ketone precursor: Koether, M. (1953). Bull. Soc. Chim. Fr: 702-705)

C.66 2-(4-methylthiazol-2-yl)but-3-yn-2-ol (ketone precursor: Aldrich 656313)

C.67 2-(4,5-dimethylthiazol-2-yl)but-3-yn-2-ol (ketone precursor: [7531-76-2])

C.68 2-(2-methylthiazol-4-yl)but-3-yn-2-ol (ketone precursor [23002-78-0])

C.69 2-(2,4-dimethylthiazol-5-yl)but-3-yn-2-ol (ketone precursor [38295-60-6])

### Example 1

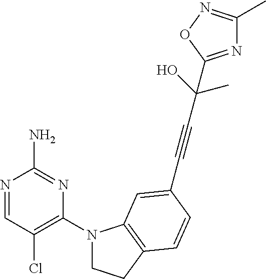

### 4-(1-(2-amino-5-chloropyrimidin-4-yl)indolin-6-yl)-2-(3-methyl-1,2,4-oxadiazol-5-yl)but-3-yn-2-ol

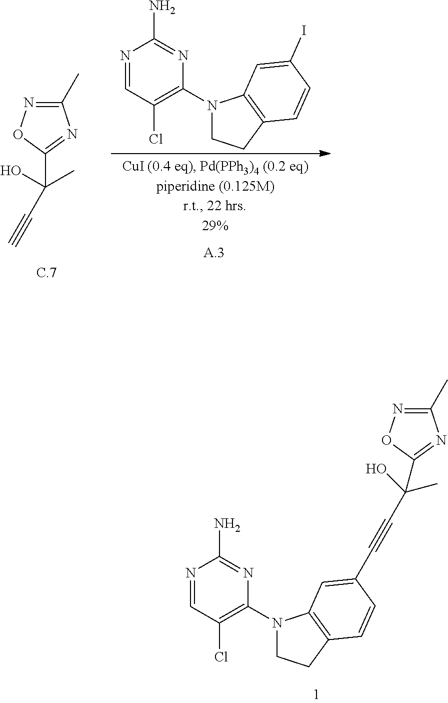

4-(1-(2-amino-5-chloropyrimidin-4-yl)indolin-6-yl)-2-(3-methyl-1,2,4-oxadiazol-5-yl)but-3-yn-2-ol (1): 5-chloro-4-(6-iodoindolin-1-yl)pyrimidin-2-amine (A.3) (100 mg, 0.27 mmol), CuI (21 mg, 0.11 mmol) and tetrakis(triphenylphosphine)palladium (64 mg, 0.055 mmol) was stirred under argon and 1.5 mL piperidine (previously degassed with argon) was added. To the stirring solution was added 2-(3-methyl-1,2,4-oxadiazol-5-yl)but-3-yn-2-ol (C.7) 61 mg, 0.4 mmol) in 1.5 mL piperidine, and the flask rinsed with 1 mL additional piperidine. The reaction stirred at room temperature for 22 hours. The mixture was condensed in vacuo and the residue purified by flash chromatography (10-100% gradient 89:9:1 dichloromethane-methanol-NH₄OH in dichloromethane) to give 31 mg 4-(1-(2-amino-5-chloropyrimidin-4-yl)indolin-6-yl)-2-(3-methyl-1,2,4-oxadiazol-5-yl)but-3-yn-2-ol (1). 1H NMR (500 MHz, DMSO-d₆) δ ppm 8.11 (1H, s), 7.24 (1H, d, J=7.3 Hz), 7.21 (1H, s), 7.09 (1H, s), 6.98 (1H, d, J=7.6 Hz), 6.66 (2H, s), 4.18 (2H, t, J=8.2 Hz), 3.11 (2H, t, J=7.8 Hz), 2.37 (3H, s), 1.88 (3H, s) Mass Spectrum (ESI) m/e=397.0, 399.1 (M+1)

Examples 2-23 were prepared using same or analogous synthetic techniques and substituting with appropriate reagent as in example 1.

### Example 2

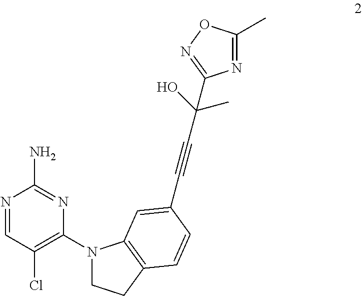

4-(1-(2-amino-5-chloropyrimidin-4-yl)indolin-6-yl)-2-(5-methyl-1,2,4-oxadiazol-3-yl)but-3-yn-2-ol (2): (from components A.3 and C.8). 1H NMR (400 MHz, DMSO-d₆) δ ppm 8.12 (1 H, s), 7.23 (1H, d, J=7.8 Hz), 7.17 (1H, s), 6.95 (1H, d, J=7.4 Hz), 6.67 (2H, s), 6.65 (1H, s), 4.17 (2H, t, J=8.0 Hz), 3.10 (2H, t, J=7.8 Hz), 2.60 (3H, s), 1.80 (3H, s) Mass Spectrum (ESI) m/e=397.1, 399.1 (M+1)

### Example 3

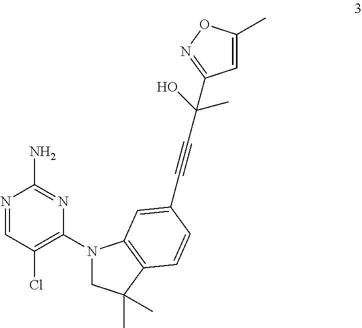

4-(1-(2-amino-5-chloropyrimidin-4-yl)-3,3-dimethylindolin-6-yl)-2-(5-methylisoxazol-3-yl)but-3-yn-2-ol (3): (from components A.15 and C.1).

1H NMR (400 MHz, DMSO-d₆) δ ppm 8.13 (1H, s), 7.21 (1H, d, J=7.4 Hz), 7.15 (1H, s), 6.99 (1H, d, J=7.8 Hz), 6.66 (2H, s), 6.41 (1H, s), 6.32 (1H, s), 3.90 (2H, s), 2.39 (3H, s), 1.77 (3H, s), 1.28 (6H, s) Mass Spectrum (ESI) m/e=424.1, 426.1 (M+1)

### Example 4

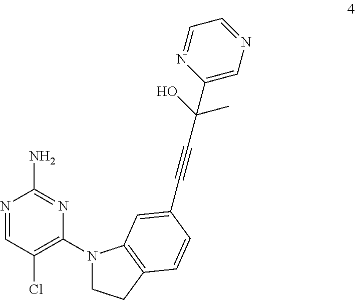

4-(1-(2-amino-5-chloropyrimidin-4-yl)indolin-6-yl)-2-(pyrazin-2-yl)but-3-yn-2-ol (4): (from components A.3 and C.9).

Mass Spectrum (ESI) m/e=393.1/395.1 (M+1)

### Example 5

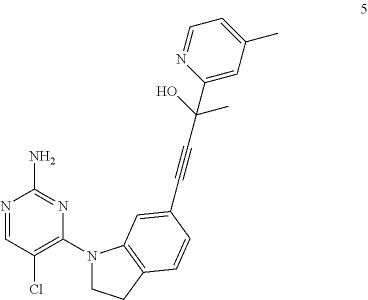

4-(1-(2-amino-5-chloropyrimidin-4-yl)indolin-6-yl)-2-(4-methylpyridin-2-yl)but-3-yn-2-ol (5): (from components A.3 and C.10).

1H NMR (400 MHz, DMSO-d₆) δ ppm 8.38 (1H, d, J=4.7 Hz), 8.10 (1H, s), 7.57 (1H, s), 7.20 (1H, d, J=7.8 Hz), 7.13 (1H, d, J=3.9 Hz), 7.10 (1H, s), 6.90 (1H, d, J=7.0 Hz), 6.66 (2 H, s), 6.22 (1H, s), 4.15 (2H, t, J=7.8 Hz), 3.08 (2H, t, J=7.8 Hz), 2.35 (3H, s), 1.76 (3H, s)

Mass Spectrum (ESI) m/e=(M+1)

### Example 6

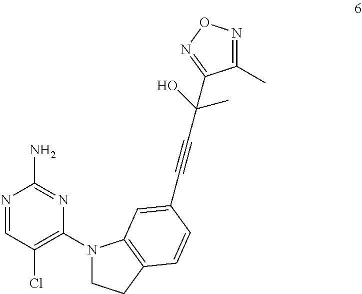

4-(1-(2-amino-5-chloropyrimidin-4-yl)indolin-6-yl)-2-(4-methyl-1,2,5-oxadiazol-3-yl)but-3-yn-2-ol (6): (from components A.3 and C.11).

1H NMR (400 MHz, DMSO-d₆) δ ppm 8.11 (1H, s), 7.24 (1H, d, J=8.2 Hz), 7.21 (1H, s), 6.98 (1H, d, J=7.0 Hz), 6.80 (1H, s), 6.67 (2H, s), 4.17 (2H, t, J=8.2 Hz), 3.10 (2H, t, J=8.2 Hz), 2.53 (3H, s), 1.92 (3H, s) Mass Spectrum (ESI) m/e=397.1, 399.1 (M+1)

### Example 7

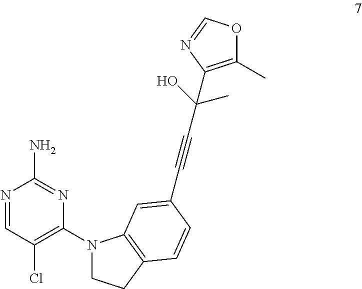

4-(1-(2-amino-5-chloropyrimidin-4-yDindolin-6-yl)-2-(5-methyloxazol-4-yl)but-3-yn-2-ol (7): (from components A.3 and C.12).

1H NMR (500 MHz, DMSO-d₆) δ ppm 8.11 (1H, s), 7.22 (1H, d, J=7.3 Hz), 7.14 (1H, s), 6.94 (1H, d, J=7.6 Hz), 6.65 (2H, s), 6.01 (1H, s), 4.16 (2H, t, J=8.2 Hz), 3.09 (2H, t, J=7.5 Hz), 2.47 (3H, s), 1.74 (3H, s) Mass Spectrum (ESI) m/e=428.0, 430.0 (M+1)

### Example 8

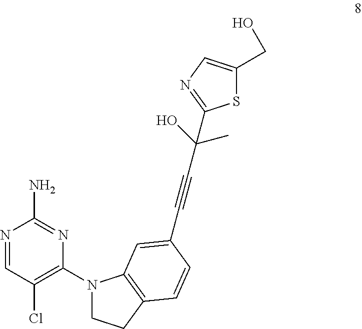

4-(1-(2-amino-5-chloropyrimidin-4-yl)indolin-6-yl)-2-(5-(hydroxymethyl)thiazol-2-yl)but-3-yn-2-ol (8): (from components A.3 and C.13).

1H NMR (500 MHz, DMSO-d₆) δ ppm 8.11 (1H, s), 7.55 (1H, s), 7.22 (1H, d, J=6.4 Hz), 7.13 (1H, s), 6.93 (1H, d, J=6.8 Hz), 6.89 (1H, s), 6.65 (2H, s), 5.50 (1H, s), 4.63 (2H, s), 4.16 (2H, s), 3.07-3.12 (2H, m), 1.83 (3H, s)

### Example 9

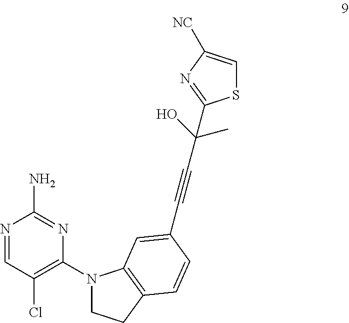

2-(4-(1-(2-amino-5-chloropyrimidin-4-yl)indolin-6-yl)-2-hydroxybut-3-yn-2-yl)thiazole-4-carbonitrile (9): (from components A.3 and C.14).

1H NMR (500 MHz, DMSO-d₆) δ ppm 8.82 (1H, s), 7.36 (1H, s), 7.17-7.25 (2H, m), 6.97 (1H, s), 6.67 (2H, s), 4.18 (2H, s), 3.11 (2H, s), 1.86 (3H, s)

### Example 10

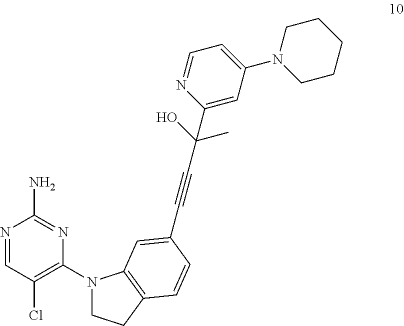

4-(1-(2-amino-5-chloropyrimidin-4-yl)indolin-6-yl)-2-(4-(piperidin-1-yl)pyridin-2-yl)but-3-yn-2-ol (10): (from components A.3 and C.15).

1H NMR (500 MHz, DMSO-d₆) δ ppm 8.14 (1H, d, J=7.6 Hz), 8.12 (1H, s), 7.57 (1H, d, J=2.0 Hz), 7.39-7.47 (2H, m), 7.10 (2H, d, J=7.6 Hz), 6.65 (2H, s), 6.44 (1H, s), 6.22 (1H, s), 4.22-4.33 (2H, m), 3.67-3.88 (4H, m), 3.20 (2H, t, J=8.2 Hz), 1.59-1.75 (9H, m)

### Example 11

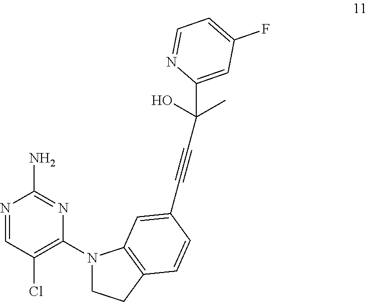

4-(1-(2-amino-5-chloropyrimidin-4-yl)indolin-6-yl)-2-(4-fluoropyridin-2-yl)but-3-yn-2-ol (11): (from components A.3 and C.15).

1H NMR (400 MHz, DMSO-d₆) δ ppm 8.11 (1H, s), 7.72 (1H, d, J=7.4 Hz), 7.39 (1H, d, J=7.8 Hz), 7.35 (1H, s), 7.04 (1H, d, J=7.0 Hz), 6.64 (2H, s), 6.37 (1H, d, J=2.3 Hz), 5.99-6.09 (2H, m), 4.24 (2H, t, J=7.8 Hz), 3.17 (2H, t, J=8.0 Hz), 1.52 (3H, s)

### Example 12

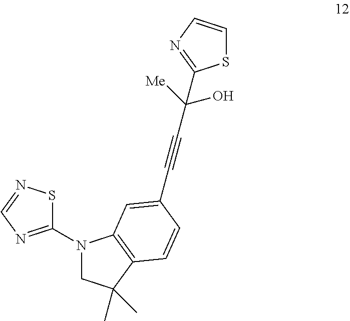

4-(3,3-dimethyl-1-(1,2,4-thiadiazol-5-yl)indolin-6-yl)-2-(thiazol-2-yl)but-3-yn-2-ol (12): (from components A.81 and C.4 using).

1H NMR (400 MHz, DMSO-d₆) δ ppm 8.38 (1H, s), 7.97 (1H, s), 7.78 (1H, d, J=2.7 Hz), 7.69 (1H, d, J=2.7 Hz), 7.36 (1H, d, J=7.8 Hz), 7.15 (1H, d, J=7.4 Hz), 7.06 (1H, s), 3.87 (2 H, s), 1.88 (3H, s), 1.38 (6H, s)

### Example 13

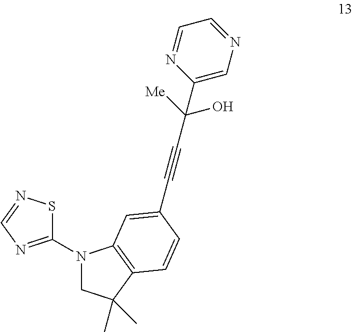

4-(3,3-dimethyl-1-(1,2,4-thiadiazol-5-yl)indolin-6-yl)-2-(pyrazin-2-yl)but-3-yn-2-ol (13): (from components A.81 and C.14).

1H NMR (400 MHz, DMSO-d₆) δ ppm 9.01 (1H, s), 8.64 (2H, dd, J=10.4, 1.8 Hz), 8.38 (1H, s), 7.96 (1H, s), 7.35 (1H, d, J=7.4 Hz), 7.14 (1H, d, J=7.8 Hz), 6.68 (1H, s), 3.87 (2H, s), 1.84 (3H, s), 1.38 (6H, s)

### Example 14

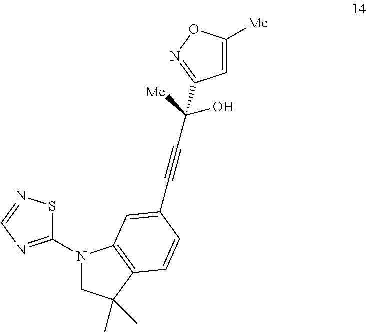

(R)-4-(3,3-dimethyl-1-(1,2,4-thiadiazol-5-yl)indolin-6-yl)-2-(5-methylisoxazol-3-yl)but-3-yn-2-ol (14): (from components A.81 and C.3).

1H NMR (500 MHz, DMSO-d₆) δ ppm 8.38 (1H, s), 7.97 (1H, s), 7.36 (1H, d, J=7.3 Hz), 7.15 (1H, d, J=7.3 Hz), 6.53 (1H, s), 6.35 (1H, s), 3.87 (2H, s), 2.41 (3H, s), 1.79 (3H, s), 1.38 (6H, s)

### Example 15

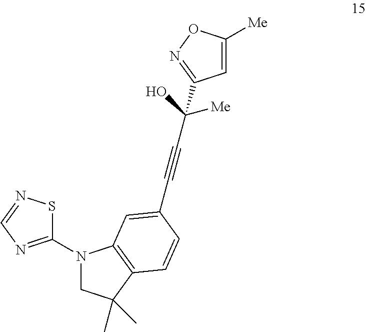

(S)-4-(3,3-dimethyl-1-(1,2,4-thiadiazol-5-yl)indolin-6-yl)-2-(5-methylisoxazol-3-yl)but-3-yn-2-ol (15): (from components A.81 and C.2).

1H NMR (500 MHz, DMSO-d₆) δ ppm 8.39 (1H, s), 7.97 (1H, s), 7.36 (1H, d, J=7.6 Hz), 7.15 (1H, d, J=7.3 Hz), 6.53 (1H, s), 6.35 (1H, s), 3.87 (2H, s), 2.41 (3H, s), 1.79 (3H, s), 1.38 (6H, s)

### Example 16

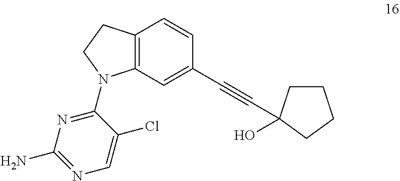

1-(2-(1-(2-amino-5-chloropyrimidin-4-yl)indolin-6-yl)ethynyl)cyclopentanol (16): (from components A.3 and C.36).

¹H NMR (methanol-d₄) δ 8.13 (s, 1H), 8.02 (s, 1H), 7.32 (d, J=8.0 Hz, 1H), δ 7.21 (d, J=8.0 Hz, 1H), 4.61 (t, J=8.0 Hz, 2H), 3.25 (t, J=8.0 Hz, 2H), 2.10-1.96 (m, 4H), 1.90-1.70 (m, 4H); ms 355.1 (M+H⁺).

### Example 17

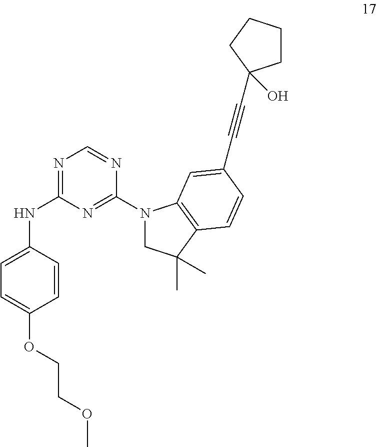

1-(2-(1-(4-(4-(2-methoxyethoxy)phenylamino)-1,3,5-triazin-2-yl)-3,3-dimethylindolin-6-yl)ethynyl)cyclopentanol (17): (from components A.93 and C.36).

¹H NMR (400 MHz, DMSO-d₆) δ ppm 9.76 (1H, s), 8.39 (2H, d, J=40.3 Hz), 7.60 (2H, s), 7.26 (1H, d, J=7.7 Hz), 7.05 (1H, dd, J=7.5, 1.3 Hz), 6.94 (2H, d, J=7.7 Hz), 5.28 (1H, s), 4.07 (2H, dd, J=5.5, 4.0 Hz), 3.96 (2H, s), 3.65 (2H, dd, J=5.3, 3.8 Hz), 3.30 (3H, s), 1.81-1.97 (4H, m), 1.62-1.80 (4H, m), 1.33 (6H, s); Mass Spectrum (ESI) m/e=500.5 [M+1].

### Example 18

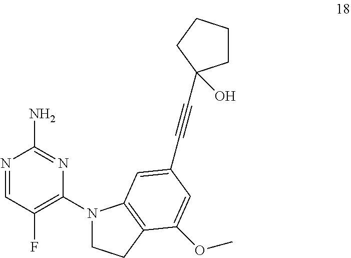

1-(2-(1-(2-amino-5-fluoropyrimidin-4-yl)-4-methoxyindolin-6-yl)ethynyl)cyclopentanol (18). (from components A.87 and C.36) ¹H NMR (500 MHz, DMSO-d₆) δ ppm 7.60 (1H, s), 6.61 (1H, s), 6.36 (2H, s), 5.75 (1H, s), 5.24 (1H, s), 4.16-4.24 (2H, m), 3.80 (3H, s), 3.01 (2H, t, J=8.7 Hz), 1.88-1.94 (4H, m), 1.63-1.78 (4H, m); Mass Spectrum (ESI) m/e=369.1 [M+1].

### Example 19

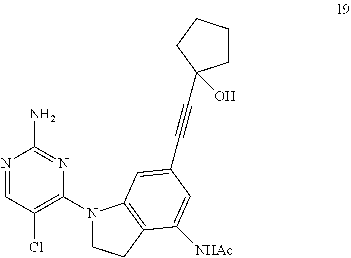

N-(1-(2-amino-5-chloropyrimidin-4-yl)-6-(2-(1-hydroxycyclopentyl)ethynyl)indolin-4-yl)acetamide (19). (from components A.90 and C.36) ¹H NMR (500 MHz, DMSO-d₆) δ ppm 9.43 (1H, s), 8.11 (1H, s), 7.31 (1H, s), 6.88 (1H, s), 6.66 (2H, s), 5.27 (1H, s), 4.15 (2H, t, J=8.2 Hz), 3.02 (2H, t, J=8.2 Hz), 2.07 (3H, s), 1.80-1.93 (4H, m), 1.61-1.76 (4H, m); Mass Spectrum (ESI) m/e=412.1 [M+1].

### Example 20

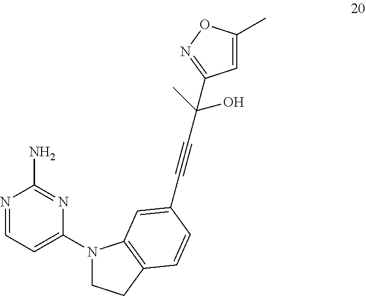

4-(1-(2-aminopyrimidin-4-yl)indolin-6-yl)-2-(5-methylisoxazol-3-yl)but-3-yn-2-ol (20). (from components A.95 and C.1) ¹H NMR (400 MHz, DMSO-d₆) δ ppm 8.43 (1H, s), 7.99 (1 H, s), 7.19 (1H, d, J=7.8 Hz), 6.96 (1H, dd, J=7.6, 1.4 Hz), 6.28-6.42 (4H, m), 6.04 (1H, d, J=5.5 Hz), 3.97 (2H, t, J=8.6 Hz), 3.17 (2H, t, J=8.6 Hz), 2.40 (3H, s), 1.81 (3H, s); Mass Spectrum (ESI) m/e=362.4 [M+1].

### Example 21

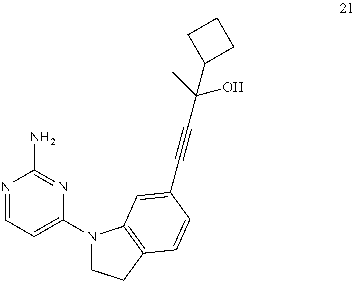

4-(1-(2-aminopyrimidin-4-yl)indolin-6-yl)-2-cyclobutylbut-3-yn-2-ol (21). (from components A.95 and C.22) ¹H NMR (400 MHz, DMSO-d₆) δ ppm 8.39 (1H, s), 8.01 (1H, s), 7.18 (1H, d, J=7.4 Hz), 6.94 (1H, dd, J=7.4, 1.2 Hz), 6.27 (2H, s), 6.05 (1H, s), 5.18 (1H, s), 3.97 (2H, t, J=8.6 Hz), 3.16 (2H, t, J=8.6 Hz), 2.52-2.56 (1H, m), 1.64-2.14 (6H, m), 1.32 (3H, s); Mass Spectrum (ESI) m/e=335.2 [M+1].

### Example 22

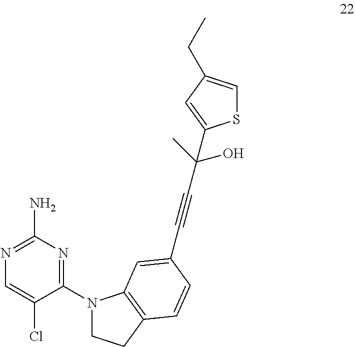

4-(1-(2-amino-5-chloropyrimidin-4-yl)indolin-6-yl)-2-(4-ethylthiophen-2-yl)but-3-yn-2-ol (22). (from components A.2 and C.26) ¹H NMR (500 MHz, DMSO-d₆) δ ppm 8.12 (1H, s), 7.23 (1H, d, J=7.8 Hz), 7.17 (1H, s), 6.94-7.03 (3H, m), 6.66 (2H, s), 6.37 (1H, s), 4.17 (2 H, t, J=8.3 Hz), 3.10 (2H, t, J=7.8 Hz), 2.51-2.58 (2H, m), 1.78 (3H, s), 1.16 (3H, t, J=7.5 Hz); Mass Spectrum (ESI) m/e=425.2 [M+1].

### Example 23

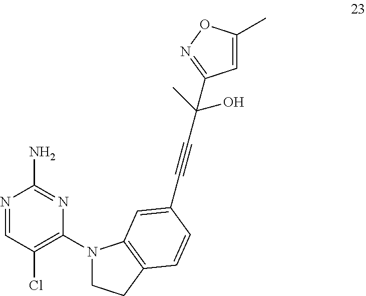

4-(1-(2-amino-5-chloropyrimidin-4-yl)indolin-6-yl)-2-(5-methylisoxazol-3-yl)but-3-yn-2-ol (23). (from components A.2 and C.1) ¹H NMR (400 MHz, DMSO-d₆) δ ppm 8.11 (1H, s), 7.22 (1H, d, J=7.4 Hz), 7.17 (1H, s), 6.95 (1H, d, J=7.4 Hz), 6.65 (2H, s), 6.42 (1H, s), 6.33 (1H, s), 4.17 (2H, t, J=8.2 Hz), 3.10 (2H, t, J=8.2 Hz), 2.39 (3H, s), 1.77 (3H, s); Mass Spectrum (ESI) m/e=396.1 [M+1].

### Example 24 and 25

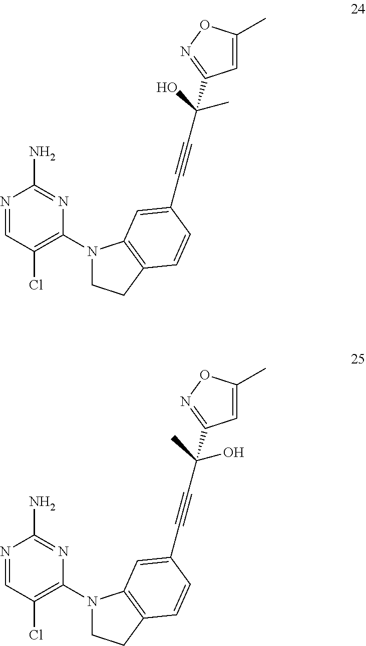

(S)-4-(1-(2-amino-5-chloropyrimidin-4-yl)indolin-6-yl)-2-(5-methylisoxazol-3-yl)but-3-yn-2-ol (24) and (R)-4-(1-(2-amino-5-chloropyrimidin-4-yl)indolin-6-yl)-2-(5-methylisoxazol-3-yl)but-3-yn-2-ol (25). The racemic mixture (23, 0.1 g) was separated on a preparative chiral column (AD-H Chiralpak, 250×20 mm) using 40% of isopropanol in hexane. The first eluted enantiomer (24) was assigned as (S)-4-(1-(2-amino-5-chloropyrimidin-4-yl)indolin-6-yl)-2-(5-methylisoxazol-3-yl)but-3-yn-2-ol (0.033 g) as a colorless syrup; the second eluted enantiomer (25) was assigned as (R)-4-(1-(2-amino-5-chloropyrimidin-4-yl)indolin-6-yl)-2-(5-methylisoxazol-3-yl)but-3-yn-2-ol (0.0325 g) as a white foam: ¹H NMR (400 MHz, DMSO-d₆) δ ppm 8.11 (1H, s), 7.22 (1H, d, J=7.4 Hz), 7.16 (1H, d, J=0.8 Hz), 6.95 (1H, dd, J=7.4, 1.6 Hz), 6.65 (2H, s), 6.41 (1H, s), 6.33 (1H, d, J=0.8 Hz), 4.17 (2H, t, J=8.2 Hz), 3.10 (2H, t, J=8.4 Hz), 2.39 (3H, s), 1.77 (3H, s); Mass Spectrum (ESI) m/e=396.1 [M+1]. Stereochemical assignment based on biological activity and analogy to compounds of defined structure.

### Example 26

Examples 26, 27 were prepared using same or analogous synthetic techniques and substituting with appropriate reagent as in example 1.

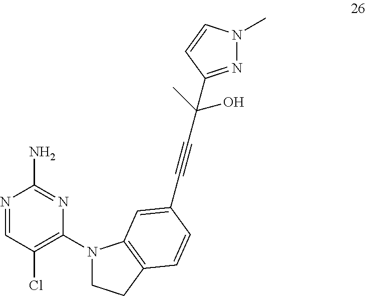

4-(1-(2-amino-5-chloropyrimidin-4-yl)indolin-6-yl)-2-(1-methyl-1H-pyrazol-3-yl)but-3-yn-2-ol (26). (from components A.2 and C.23) ¹H NMR (400 MHz, DMSO-d₆) δ ppm 8.12 (1 H, s), 7.57 (1H, d, J=2.0 Hz), 7.20 (1H, d, J=7.4 Hz), 7.12 (1H, s), 6.92 (1H, dd, J=7.4, 1.2 Hz), 6.64 (2H, s), 6.29 (1H, d, J=2.0 Hz), 5.87 (1H, s), 4.16 (2H, t, J=8.2 Hz), 3.80 (3H, s), 3.09 (2H, t, J=8.2 Hz), 1.75 (3H, s); Mass Spectrum (ESI) m/e=395.2 [M+1].

### Example 27

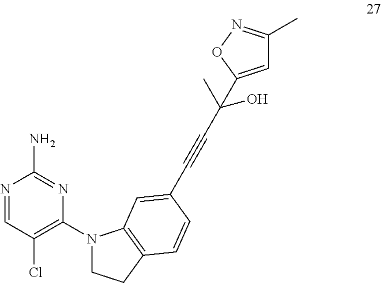

4-(1-(2-amino-5-chloropyrimidin-4-yl)indolin-6-yl)-2-(3-methylisoxazol-5-yl)but-3-yn-2-ol (27). (from components A.2 and C.24) ¹H NMR (400 MHz, DMSO-d₆) δ ppm 8.11 (1H, s), 7.18-7.26 (2H, m), 6.98 (1H, dd, J=7.6, 1.4 Hz), 6.65 (2H, s), 6.63 (1H, s), 6.35 (1H, s), 4.17 (2H, t, J=8.2 Hz), 3.11 (2H, t, J=8.2 Hz), 2.22 (3H, s), 1.77 (3H, s); Mass Spectrum (ESI) m/e=396.1 [M+1].

### Example 28 and 29

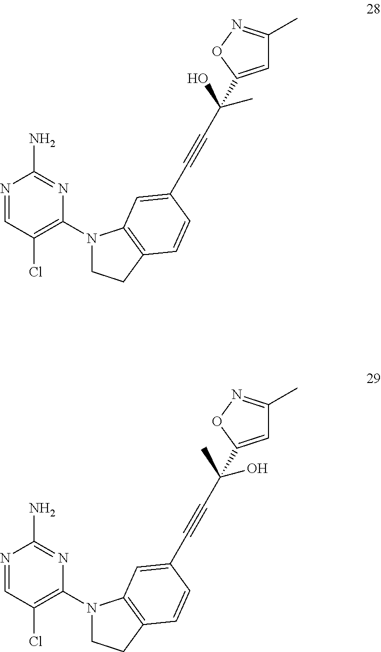

(S)-4-(1-(2-amino-5-chloropyrimidin-4-yl)indolin-6-yl)-2-(3-methylisoxazol-5-yl)but-3-yn-2-ol (28) and (R)-4-(1-(2-amino-5-chloropyrimidin-4-yl)indolin-6-yl)-2-(3-methylisoxazol-5-yl)but-3-yn-2-ol (29). The racemic mixture (27) (0.0978 g) was separated on a preparative chiral column (AD-H Chiralpak, 250×20 mm) using 40% of isopropanol in hexane. The first eluted enantiomer (28) was assigned as (S)-4-(1-(2-amino-5-chloropyrimidin-4-yl)indolin-6-yl)-2-(3-methylisoxazol-5-yl)but-3-yn-2-ol (0.033 g) as a colorless syrup; the second eluted enantiomer (29) was assigned as (R)-4-(1-(2-amino-5-chloropyrimidin-4-yl)indolin-6-yl)-2-(3-methylisoxazol-5-yl)but-3-yn-2-ol (0.0325 g) as a white foam: 1H NMR (400 MHz, CHLOROFORM-d) δ ppm 8.11 (1H, s), 7.17-7.26 (2H, m), 6.98 (1H, dd, J=7.6, 1.4 Hz), 6.65 (2H, s), 6.63 (1H, s), 6.35 (1H, s), 4.17 (2H, t, J=8.4 Hz), 3.11 (2H, t, J=8.2 Hz), 2.22 (3H, s), 1.77 (3H, s); Mass Spectrum (ESI) m/e=396.1 [M+1].

### Example 30 and 31

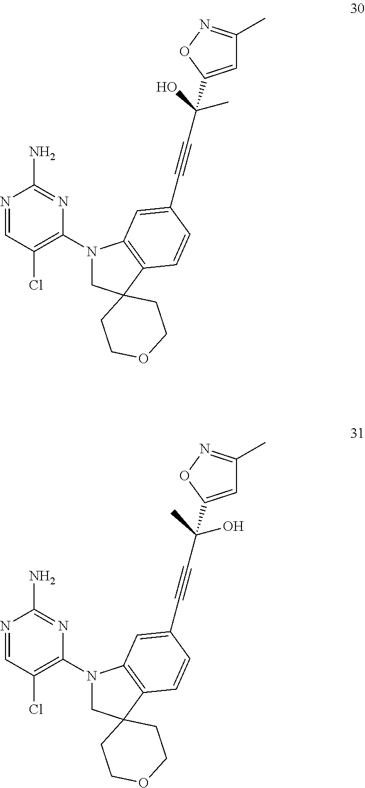

(2S)-4-(1-(2-amino-5-chloro-4-pyrimidinyl)-1,2,2′,3′,5′,6′-hexahydrospiro[indole-3,4′-pyran]-6-yl)-2-(3-methyl-5-isoxazolyl)-3-butyn-2-ol (30) and (2R)-4-(1-(2-amino-5-chloro-4-pyrimidinyl)-1,2,2′,3′,5′,6′-hexahydrospiro[indole-3,4′-pyran]-6-yl)-2-(3-methyl-5-isoxazolyl)-3-butyn-2-ol (31). The racemic mixture, prepared from components A.101 and C.24 by the procedure of example 1, was separated on a preparative chiral column (OD-H Chiralpak, 250×20 mm) using 20% of isopropanol in hexane. The first eluted enantiomer (30) was assigned as (2S)-4-(1-(2-amino-5-chloro-4-pyrimidinyl)-1,2,2′,3′,5′,6′-hexahydrospiro[indole-3,4′-pyran]-6-yl)-2-(3-methyl-5-isoxazolyl)-3-butyn-2-ol (0.0337 g, 11.4% yield) as a brown solid; the second eluted enantiomer (31) was assigned as (2R)-4-(1-(2-amino-5-chloro-4-pyrimidinyl)-1,2,2′,3′,5′,6′-hexahydrospiro[indole-3,4′-pyran]-6-yl)-2-(3-methyl-5-isoxazolyl)-3-butyn-2-ol (0.0254 g, 8.6% yield) as a brown solid: ¹H NMR (400 MHz, DMSO-d₆) δ ppm 8.14 (1H, s), 7.29 (1H, d, J=7.8 Hz), 7.13 (1H, s), 7.02 (1H, dd, J=7.6, 1.4 Hz), 6.70 (2H, s), 6.63 (1H, s), 6.35 (1H, s), 4.14 (2H, s), 3.84 (2H, dd, J=11.9, 2.5 Hz), 3.47 (2H, t, J=11.3 Hz), 2.22 (3H, s), 1.83-1.95 (2H, m), 1.77 (3H, s), 1.56 (2H, d, J=13.3 Hz); Mass Spectrum (ESI) m/e=466.1 [M+1].

### Example 32

Examples 32-39 were prepared using same or analogous synthetic techniques and substituting with appropriate reagent as in example 1.

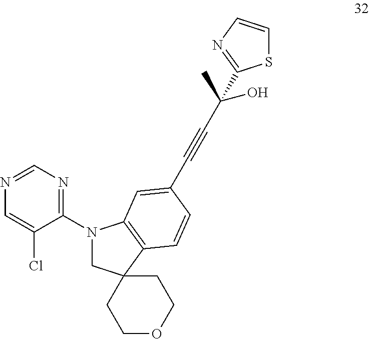

(2R)-4-[1-(5-chloropyrimidin-4-yl)-1,2,2′,3′,5′,6′-hexahydrospiro[indole-3,4′-pyran]-6-yl]-2-(1,3-thiazol-2-yl)but-3-yn-2-ol (32). (from components A.103 and C.6) ¹H NMR (500 MHz, DMSO-d₆) δ ppm 8.70 (2H, d), 7.76 (1H, s), 7.67 (1H, d, J=2.0 Hz), 7.45 (1H, s), 7.34 (1H, d, J=7.6 Hz), 7.07 (1H, d, J=7.8 Hz), 7.01 (1H, s), 4.31 (2H, s), 3.84 (2H, dd, J=11.9, 3.3 Hz), 3.47 (2H, t, J=11.5 Hz), 1.77-1.94 (5H, m), 1.56 (2H, d, J=13.0 Hz); Mass Spectrum (ESI) m/e=453.1 [M+1].

### Example 33

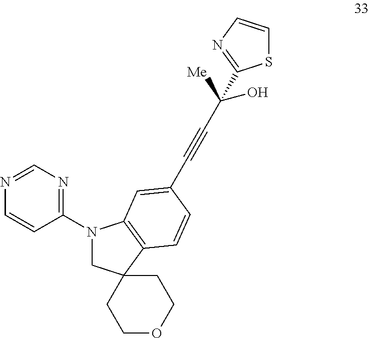

(2R)-4-(1-pyrimidin-4-yl-1,2,2′,3′,5′,6′-hexahydrospiro[indole-3,4′-pyran]-6-yl)-2-(1,3-thiazol-2-yl)but-3-yn-2-ol (33). (from components A.106 and C.6) ¹H NMR (500 MHz, DMSO-d₆) δ ppm 8.50 (1H, s), 7.78 (1H, s), 7.69 (1H, s), 7.33 (1H, d, J=7.1 Hz), 7.07 (1H, d, J=3.9 Hz), 7.04 (1H, s), 4.04 (2H, s), 3.87 (2H, d, J=9.5 Hz), 3.57 (2H, t, J=12.0 Hz), 1.82-1.99 (5H, m), 1.57 (2H, d, J=13.0 Hz); Mass Spectrum (ESI) m/e=418.51 [M+1].

### Example 34

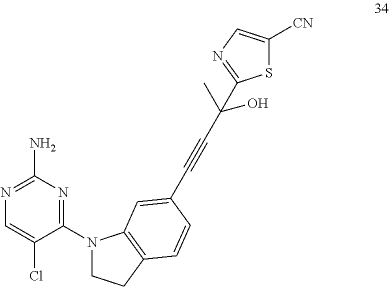

2-(4-(1-(2-amino-5-chloropyrimidin-4-yl)indolin-6-yl)-2-hydroxybut-3-yn-2-yl)thiazole-5-carbonitrile (34). (from components A.2 and C.14) ¹H NMR (400 MHz, DMSO-d₆) δ ppm 8.62 (1H, s), 8.12 (1H, s), 7.52 (1H, s), 7.23 (1H, d, J=7.8 Hz), 7.17 (1H, s), 6.95 (1H, d, J=7.8 Hz), 6.68 (2H, s), 4.16 (2H, t, J=8.2 Hz), 3.10 (2H, t, J=8.0 Hz), 1.87 (3H, s); Mass Spectrum (ESI) m/e=423.1 [M+1].

### Example 35

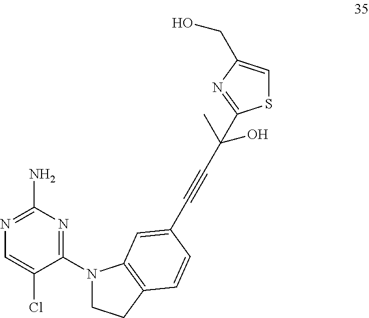

4-(1-(2-amino-5-chloropyrimidin-4-yl)indolin-6-yl)-2-(4-(hydroxymethyl)thiazol-2-yl)but-3-yn-2-ol (35). (from components A.2 and C.26) ¹H NMR (500 MHz, DMSO-d₆) δ ppm 8.11 (1H, s), 7.35 (1H, d, J=1.0 Hz), 7.22 (1H, d, J=7.8 Hz), 7.15 (1H, s), 6.87-6.97 (2H, m), 6.66 (2H, s), 5.29 (1H, t, J=5.7 Hz), 4.53 (2H, d, J=5.9 Hz), 4.16 (2H, t, J=7.9 Hz), 3.10 (2 H, t, J=8.1 Hz), 1.84 (3H, s); Mass Spectrum (ESI) m/e=428.0 [M+1].

### Example 36

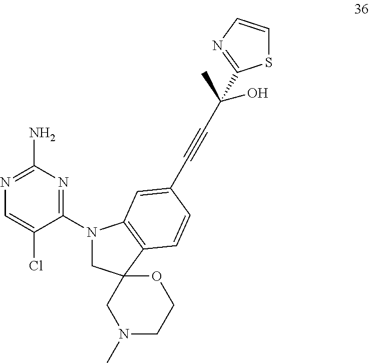

(2R)-4-[1-(2-amino-5-chloropyrimidin-4-yl)-4′-methyl-1,2-dihydrospiro[indole-3,2′-[1,4]oxazinan]-6-yl]-2-(1,3-thiazol-2-yl)but-3-yn-2-ol (36). (from components A.114 and C.6) ¹H NMR (500 MHz, DMSO-d₆) δ ppm 8.14 (1H, s), 7.76 (1H, d, J=3.2 Hz), 7.67 (1H, d, J=3.2 Hz), 7.54 (1H, d, J=6.8 Hz), 7.32 (1H, s), 6.96-7.04 (2H, m), 6.70 (2H, s), 4.08-4.24 (2H, m), 3.57-3.75 (2H, m), 2.43-2.61 (3H, m), 2.32 (1H, s), 2.22 (3H, s), 1.86 (3H, s); Mass Spectrum (ESI) m/e=483.1 [M+1].

### Example 37

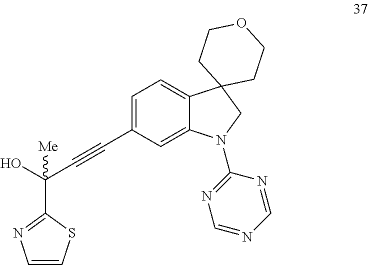

2-(1,3-thiazol-2-yl)-4-(1-(1,3,5-triazin-2-yl)-1,2,2′,3′,5′,6′-hexahydrospiro[indole-3,4′-pyran]-6-yl)-3-butyn-2-ol (37) (from components A.6 and C.4)

¹H NMR (500 MHz, (CD₃)₂CO) δ 8.70 (s, 2H), 8.37 (br m, 1H), 7.65 (br m, 1H), 7.46 (br m, 1H), 7.26 (br m, 1H), 7.08 (br m, 1H), 5.84 (s, 1H), 4.15 (s, 2H), 3.82 (br m, 2H), 3.51 (br m, 2H), 1.98 (br m, 2H) 1.89 (s, 3H), 1.52 (br m, 2H); ESI-MS: M+H⁺ 420.1 m/z; RPHPLC elutes @ 6.783 min (94%).

### Example 38

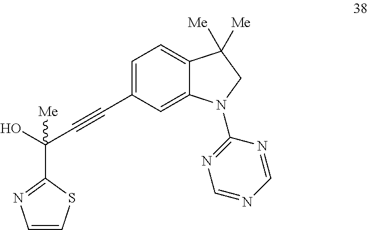

4-(3,3-dimethyl-1-(1,3,5-triazin-2-yl)-2,3-dihydro-1H-indol-6-yl)-2-(1,3-thiazol-2-yl)-3-butyn-2-ol (38): (from components A.7 and C.4). ¹H NMR (400 MHz, DMSO-d6) δ 8.89 (br s, 2H), 8.34 (d, J=1 Hz, 1H), 7.77 (d, J=3.4 Hz, 1H), 7.69 (d, J=3.25 Hz, 1H), 7.34 (d, J=7.7 Hz, 1H), 7.15 (dd, J=7.7 Hz, J=1.3 Hz, 1H), 7.06 (s, 1H), 3.99 (s, 2H), 1.88 (s, 3H), 1.34 (s, 6H); ESI-MS: M+H⁺ 378.1 m/z; RPHPLC elutes @ 7.894 min (95%).

### Example 39

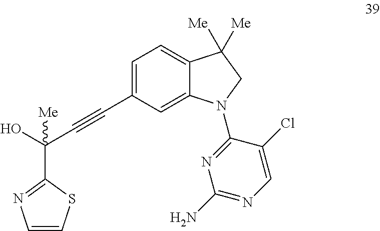

4-(1-(2-amino-5-chloro-4-pyrimidinyl)-3,3-dimethyl-2,3-dihydro-1H-indol-6-yl)-2-(1,3-thiazol-2-yl)-3-Butyn-2-ol (39) from components A.15 and C.4. ¹H NMR (400 MHz, DMSO-d6) δ 8.12 (s, 1H), 7.76 (d, J=3.2 Hz, 1H), 7.67 (d, J=3.2 Hz, 1H), 7.21 (d, J=7.6 Hz, 1H), 7.12 (d, J=1 Hz, 1H), 6.98 (dd, J=8 Hz, J=1.3 Hz, 1H), 6.93 (s, 1H), 6.65 (br s, 2H), 3.89 (s, 2H), 1.85 (s, 3H), 1.28 (s, 6H); ESI-MS: M+H⁺ 426.1 m/z; RPHPLC @ 6.602 min (95%).

### Example 40 and 41

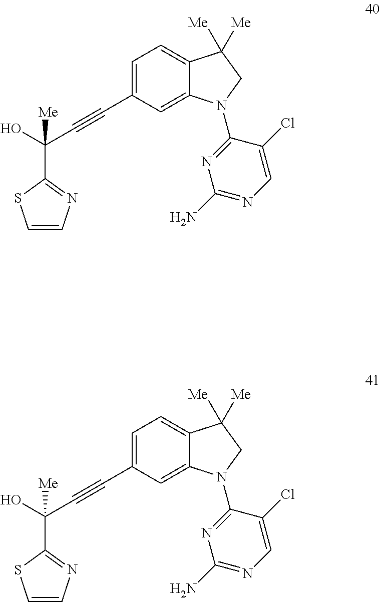

(2R)-4-(1-(2-amino-5-chloro-4-pyrimidinyl)-3,3-dimethyl-2,3-dihydro-1H-indol-6-yl)-2-(1,3-thiazol-2-yl)-3-butyn-2-ol (40) The racemic mixture (39) was separated on a preparative chiral column AD-H eluting with 18% iPrOH/hexane then with 30% iPrOH/hexane. The first peak to elute gave (2R)-4-(1-(2-amino-5-chloro-4-pyrimidinyl)-3,3-dimethyl-2,3-dihydro-1H-indol-6-yl)-2-(1,3-thiazol-2-yl)-3-butyn-2-ol (40) (76 mg, 22%)) as a tan colored solid.

¹H NMR (500 MHz, DMSO-d6) δ 8.12 (s, 1H), 7.76 (d, J=3.2 Hz, 1H), 7.67 (d, J=3.2 Hz, 1H), 7.22 (d, J=7.6 Hz, 1H), 7.13 (d, J=1 Hz, 1H), 6.99 (dd, J=7.6 Hz, J=1.1 Hz, 1H), 6.96 (s, 1H), 6.68 (br s, 2H), 3.9 (s, 2H), 1.86 (s, 3H), 1.29 (s, 6H); ESI-MS: M+H⁺ 426.1 m/z; RPHPLC @ 6.609 min (97%); [α]^{D}₂₀=−45.2° (2S)-4-(1-(2-amino-5-chloro-4-pyrimidinyl)-3,3-dimethyl-2,3-dihydro-1H-indol-6-yl)-2-(1,3-thiazol-2-yl)-3-butyn-2-ol (41) The second peak to elute gave (2S)-4-(1-(2-amino-5-chloro-4-pyrimidinyl)-3,3-dimethyl-2,3-dihydro-1H-indol-6-yl)-2-(1,3-thiazol-2-yl)-3-butyn-2-ol (41) as a tan colored solid (76 mg, 24%). Stereochemistry assigned by biological activity and analogy to compounds of defined structure.

### Example 42 and 43

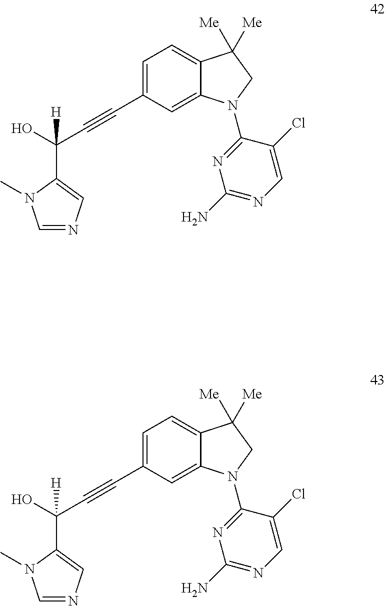

(1R*)-3-(1-(2-amino-5-chloro-4-pyrimidinyl)-3,3-dimethyl-2,3-dihydro-1H-indol-6-yl)-1-(1-methyl-1H-imidazol-5-yl)-2-propyn-1-ol (42) The racemic mixture prepared from components A.15 and C.16 by the procedure of example 1, was separated on a preparative chiral column AD-H eluting with 25% iPrOH/hexane afforded (1R*)-3-(1-(2-amino-5-chloro-4-pyrimidinyl)-3,3-dimethyl-2,3-dihydro-1H-indol-6-yl)-1-(1-methyl-1H-imidazol-5-yl)-2-propyn-1-ol (42) as an off white solid (11 mg, 22%). Stereochemistry arbitrarily assigned as R for the second peak to elute from the chiral column.

¹H NMR (500 MHz, (CDCl₃) δ 7.94 (s, 1H), 7.48 (br s, 1H), 7.19 (br s, 1H), 7.02 (br s, 1H), 7.01 (br s, 2H), 5.62 (s, 1H), 4.98 (s, 2H), 4.34 (s, 2H), 3.71 (s, 3H), 1.25 (s, 6H); ESI MS: M+H⁺ 409.1 m/z; RPHPLC @ 5.218 min (95%); Analytical chiral column AD-H (isocratic 25% iPrOH/hexane) elutes @ 13.318 min.

(1S*)-3-(1-(2-amino-5-chloro-4-pyrimidinyl)-3,3-dimethyl-2,3-dihydro-1H-indol-6-yl)-1-(1-methyl-1H-imidazol-5-yl)-2-propyn-1-ol (43): The first peak to elute from the chiral column afforded (1S*)-3-(1-(2-amino-5-chloro-4-pyrimidinyl)-3,3-dimethyl-2,3-dihydro-1H-indol-6-yl)-1-(1-methyl-1H-imidazol-5-yl)-2-propyn-1-ol (43) as an off white solid (18 mg, 36%).

### Example 44

Examples 44-48 were prepared using same or analogous synthetic techniques and substituting with appropriate reagent as in example 1.

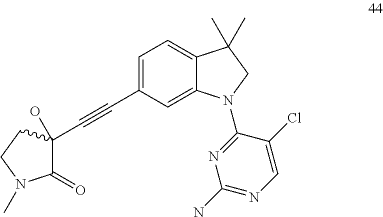

(3S)-3-((1-(2-amino-5-chloro-4-pyrimidinyl)-3,3-dimethyl-2,3-dihydro-1H-indol-6-yl)ethynyl)-3-hydroxy-1-methyl-2-pyrrolidinone (44). The title compound prepared from components A.15 and C.17 was obtained as a light brown film (116 mg, 47%).

¹H NMR (500 MHz, DMSO-d6) δ 8.05 (s, 1H), 7.15 (d, J=7.6 Hz, 1H), 7.09 (br s, 1H), 6.92 (br d, J=8.3 Hz, 1H), 6.61 (br s, 2H), 6.33 (s, 1H), 3.84 (s, 2H), 3.26 (m, 2H), 2.71 (s, 3H), 2.33 (m, 1H), 2.08 (m, 1H), 1.21 (s, 6H); ESI-MS: M+H⁺ 412.1 m/z; RPHPLC elutes @ 5.960 min (97%).

### Example 45

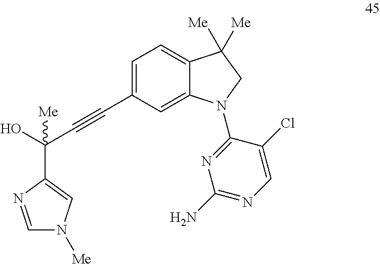

4-(1-(2-amino-5-chloro-4-pyrimidinyl)-3,3-dimethyl-2,3-dihydro-1H-indol-6-yl)-2-(1-methyl-1H-imidazol-4-yl)-3-butyn-2-ol (45). The title compound prepared from components A.15 and C.18 was obtained as a yellow solid (5 mg, 3%).

¹H NMR (500 MHz, (CDCl₃) δ 7.98 (s, 1H), 7.49 (br s, 1H), 7.19 (br s, 2H), 7.02 (br d, J=7 Hz, 1H), 6.99 (br d, J=7 Hz, 1H), 6.91 (br s, 1H), 4.93 (br s, 1H), 3.94 (s, 2H), 3.60 (s, 3H), 1.85 (s, 3H); 1.18 (s, 6H); ESI-MS: M+H⁺ 425.8 m/z; RPHPLC @ 6.633 min (97%).

### Example 46

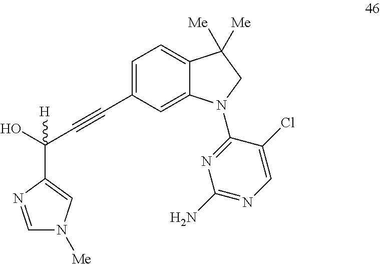

3-(1-(2-amino-5-chloro-4-pyrimidinyl)-3,3-dimethyl-2,3-dihydro-1H-indol-6-yl)-1-(1-methyl-1H-imidazol-4-yl)-2-propyn-1-ol (46) The title compound prepared from components A.15 and C.19 was obtained as a yellow solid (162 mg, 86%).

¹H NMR (500 MHz, DMSO-d6) δ 8.12 (s, 1H), 7.51 (br s, 1H), 7.22 (d, J=7.5 Hz, 1H), 7.19 (d, J=1.2 Hz, 1H), 7.13 (s, 1H), 7.02 (dd, J=7.6 Hz, J=1.2 Hz, 1H), 6.68 (br s, 2H), 5.76 (d, J=6.1 Hz, 1H), 5.41 (d, J=6.2 Hz, 1H), 3.91 (s, 2H), 3.63 (s, 3H), 1.29 (s, 6H); ESI-MS: M+H⁺409.1 m/z; RPHPLC @ 5.245 min (˜85%).

### Example 47

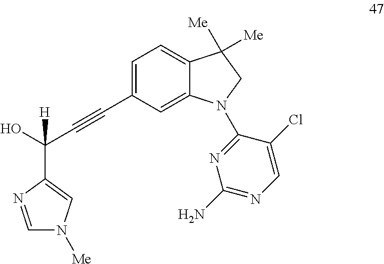

(1R*)-3-(1-(2-amino-5-chloro-4-pyrimidinyl)-3,3-dimethyl-2,3-dihydro-1H-indol-6-yl)-1-(1-methyl-1H-imidazol-4-yl)-2-propyn-1-ol (47) Separation of the enantiomers of example 46 on a preparative chiral column AD-H eluting with 30% iPrOH/hexane afforded (1R*)-3-(1-(2-amino-5-chloro-4-pyrimidinyl)-3,3-dimethyl-2,3-dihydro-1H-indol-6-yl)-1-(1-methyl-1H-imidazol-4-yl)-2-propyn-1-ol (47) as a light yellow solid (40 mg, 21%). Stereochemistry assigned as R for the second peak to elute from the chiral column based on biological activity.

### Example 48

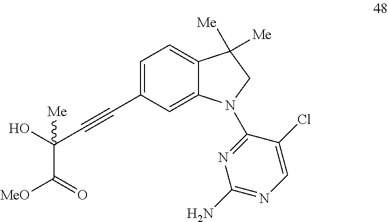

methyl 4-(1-(2-amino-5-chloro-4-pyrimidinyl)-3,3-dimethyl-2,3-dihydro-1H-indol-6-yl)-2-hydroxy-2-methyl-3-butynoate (48) (prepared from components A.15 and C.20). ¹H NMR (400 MHz, DMSO-d6) δ 8.12 (s, 1H), 7.22 (d, J=7.6 Hz, 1H), 7.15 (d, J=1 Hz, 1H), 6.99 (dd, J=7.6 Hz, J=1.2 Hz, 1H), 6.66 (s, 2H), 6.39 (s, 1H), 3.73 (s, 3H), 3.30 (s, 2H), 1.62 (s, 3H), 1.28 (s, 6H); ESI-MS: M+H⁺401.0 m/z; RPHPLC @ 6.530 min (97%).

### Example 49

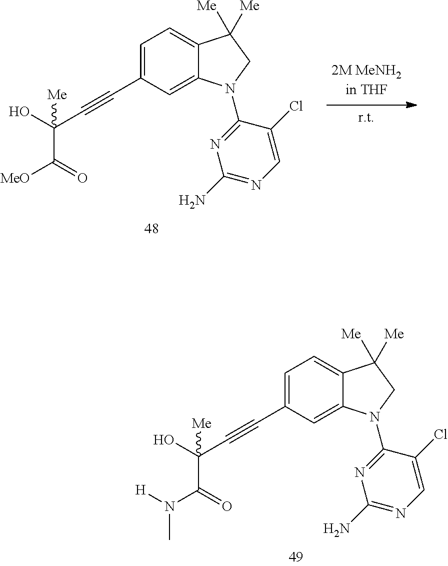

### 4-(1-(2-amino-5-chloro-4-pyrimidinyl)-3,3-dimethyl-2,3-dihydro-1H-indol-6-yl)-2-hydroxy-N,2-dimethyl-3-butynamide (49)

methyl 4-(1-(2-amino-5-chloro-4-pyrimidinyl)-3,3-dimethyl-2,3-dihydro-1H-indol-6-yl)-2-hydroxy-2-methyl-3-butynoate 48 (22 mg, 0.056 mmol) was dissolved in 2 ml of 2M Methylamine in THF. The reaction was stirred at 40° C. for 4 h was and then concentrated under vacuum. The residue was purified by CombiFlash chromatography eluting with a gradient of 40% ethyl acetate/dichloromethane to 100% ethyl acetate. The fractions containing pure product were combined and concentrated under vacuum to give 4-(1-(2-amino-5-chloro-4-pyrimidinyl)-3,3-dimethyl-2,3-dihydro-1H-indol-6-yl)-2-hydroxy-N,2-dimethyl-3-butynamide (16 mg, 71%) (49) as a white solid.

¹H NMR (500 MHz, DMSO-d6) δ 8.13 (s, 1H), 7.89 (br m, 1H), 7.22 (d, J=7.6 Hz, 1H), 7.12 (s, 1H), 6.98 (dd, J=7.8 Hz, J=1.2 Hz, 1H), 6.68 (s, 2H), 6.41 (s, 1H), 3.90 (s, 2H), 2.65 (d, J=4.6 Hz, 3H), 1.57 (s, 3H), 1.29 (s, 6H); ESI-MS: M+H⁺ 400.1 m/z; RPHPLC @ 5.820 min (97%).

### Example 50

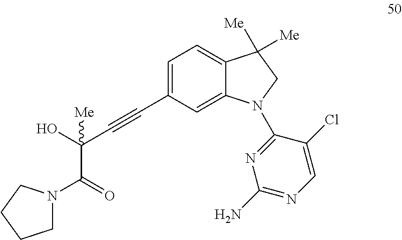

4-(1-(2-amino-5-chloro-4-pyrimidinyl)-3,3-dimethyl-2,3-dihydro-1H-indol-6-yl)-2-methyl-1-oxo-1-(1-pyrrolidinyl)-3-butyn-2-ol (50) was isolated as a side product from the synthesis of methyl 4-(1-(2-amino-5-chloro-4-pyrimidinyl)-3,3-dimethyl-2,3-dihydro-1H-indol-6-yl)-2-hydroxy-2-methyl-3-butynoate (48); the title compound was obtained as a brownish solid (38 mg, 10%).

¹H NMR (400 MHz, DMSO-d6) δ 8.11 (s, 1H), 7.21 (d, J=7.6 Hz1H), 7.15 (s, 1H), 6.99 (dd, J=7.6 Hz, J=1.2 Hz, 1H), 6.66 (s, 2H), 5.98 (s, 1H), 3.88 (s, 2H), 3.84 (m, 2H), 3.36 (m, 2H), 1.84 (m, 2H), 1.80 (m, 2H), 1.60 (s, 3H), 1.28 (s, 6H); ESI-MS: M+H⁺ 440.2 m/z; RPHPLC @ 6.726 min (97%).

### Example 51

Examples 51-56 were prepared using same or analogous synthetic techniques and substituting with appropriate reagent as in example 1.

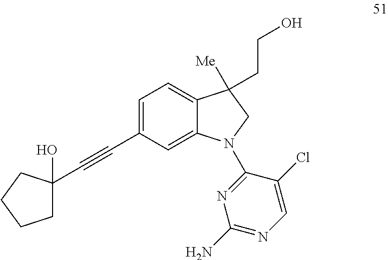

1-((1-(2-amino-5-chloro-4-pyrimidinyl)-3-(2-hydroxyethyl)-3-methyl-2,3-dihydro-1H-indol-6-yl)ethynyl)cyclopentanol (51) (prepared from components A.24 and C.36) ¹H NMR (500 MHz, DMSO-d6) δ 8.11 (s, 1H), 7.16 (d, J=7.8 Hz, 1H), 7.10 (d, J=1.5 Hz, 1H), 6.95 (dd, J=7.8 Hz, J=1.4 Hz, 1H), 6.66 (br s, 2H), 5.25 (s, 1H), 4.41 (t, J=4.9 Hz, 1H), 4.10 (d, J=10.7 Hz, 1H), 3.84 (d, J=10.7 Hz, 1H), 3.33-3.47 (m, 2H), 1.64-1.91 (m, 8H), 1.27 (s, 3H); ESI-MS: M+H⁺ 413.2 m/z; RPHPLC @ 6.110 min (95%).

### Example 52

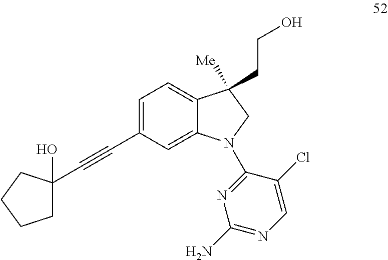

1-(03R*)-1-(2-amino-5-chloro-4-pyrimidinyl)-3-(2-hydroxyethyl)-3-methyl-2,3-dihydro-1H-indol-6-yl)ethynyl)cyclopentanol (52) The enantiomers of example 51 were separated on a preparative chiral column AD-H eluting with 15% iPrOH/hexane. the pure fractions were combined and concentrated under vacuum to give the title compound as a clear film (7 mg, 23%). The stereochemistry for the first peak to elute from the chiral column was arbitrarily assigned as R*.

¹H NMR (400 MHz, CD₃Cl₃) δ 8.01 (s, 1H), 7.55 (s, 1H), 7.08 (br m, 2H), 5.46 (br s, 2H), 4.28 (d, J=10.5 Hz, 1H), 4.04 (d, J=11.5 Hz, 1H), 3.61 (t, J=6.5 Hz, 2H), 1.96-2.07 (br m, 2H), 1.7-1.9 (br m, 8H), 1.95 (s, 3H); ESI-MS: M+H⁺413.2 m/z; RPHPLC @ 6.178 min (95%); [α]D20=-35°

### Example 53

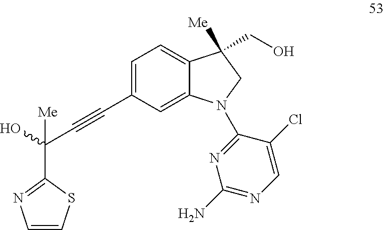

4-((3 S*)-1-(2-amino-5-chloro-4-pyrimidinyl)-3-(hydroxymethyl)-3-methyl-2,3-dihydro-1H-indol-6-yl)-2-(1,3-thiazol-2-yl)-3-butyn-2-ol (53) (prepared from components A.32 and C.4) ¹H NMR (400 MHz, DMSO-d6) δ 8.16 (br s, 1H), 7.76 (br s, 1H), 7.67 (br s, 1H), 7.19 (m, 2H), 6.96 (m, 2H), 6.64 (br s, 2H), 5.01 (t, J=5.3 Hz, 1H), 4.14 (d, J=10.3 Hz, 1H), 3.80 (d, J=10.3 Hz, 1H), 3.38 (m, 2H), 1.86 (s, 3H), 1.28 (s, 3H); ESI-MS: M+H⁺ 442.1 m/z; RPHPLC @ 5.577 min (95%). Stereochemistry is arbitrarily assigned.

### Example 54

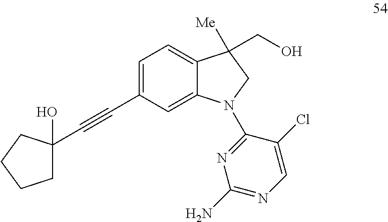

1-((1-(2-amino-5-chloro-4-pyrimidinyl)-3-(hydroxymethyl)-3-methyl-2,3-dihydro-1H-indol-6-yl)ethynyl)cyclopentanol (54) (prepared from components A.30 and C.36) ¹HNMR (500 MHz, (CD)₃SO) δ 8.10 (s, 1H), 7.17 (s, 1H), 7.16 (d, J=8.3 Hz, 1H), 6.94 (dd, J=7.4 Hz, J=1.5 Hz, 1H), 6.64 (br s, 2H), 5.24 (s, 1H), 5.00 (t, J=5.4 Hz, 1H), 4.14 (d, J=10.3 Hz, 1H), 3.80 (d, J=10.3 Hz, 1H), 3.33 (m, 2H), 1.64-1.74 (m, 4H), 1.80-1.90 (m, 4H), 1.27 (s, 3H); ESI-MS: M+H⁺ 399.1 m/z; RPHPLC @ 6.050 min (97%).

### Example 55

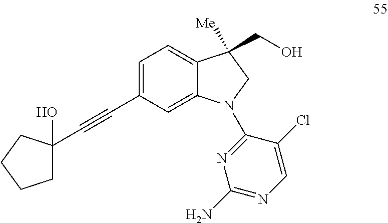

1-(((3R*)-1-(2-amino-5-chloro-4-pyrimidinyl)-3-(hydroxymethyl)-3-methyl-2,3-dihydro-1H-indol-6-yl)ethynyl)cyclopentanol (55) (prepared from components A.33 and C.36). ¹H NMR (500 MHz, DMSO-d6) δ 8.09 (s, 1H), 7.17 (s, 1H), 7.16 (d, J=8.3 Hz, 1H), 6.94 (dd, J=7.9 Hz, J=1.5 Hz, 1H), 6.64 (br s, 2H), 5.25 (s, 1H), 5.01 (t, J=5.4 Hz, 1H), 4.13 (d, J=10.3 Hz, 1H), 3.80 (d, J=10.3 Hz, 1H), 3.33 (m, 2H), 1.85-1.91 (m, 4H), 1.64-1.75 (m, 4H), 1.27 (s, 3H); ESI-MS: M+H⁺ 399.1 m/z; RPHPLC @ 6.104 min (95%); [α]D20=-40.9. Stereochemistry is arbitrarily assigned.

### Example 56

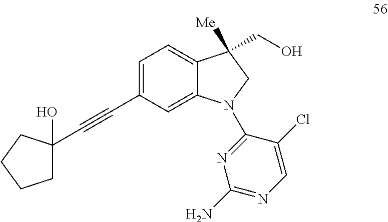

1-(((3S*)-1-(2-amino-5-chloro-4-pyrimidinyl)-3-(hydroxymethyl)-3-methyl-2,3-dihydro-1H-indol-6-yl)ethynyl)cyclopentanol (56) (prepared from components A.32 and C.36). ¹H NMR (500 MHz, DMSO-d6) δ 8.09 (s, 1H), 7.17 (s, 1H), 7.16 (d, J=8.3 Hz, 1H), 6.94 (dd, J=7.8 Hz, J=1.5 Hz, 1H), 6.64 (br s, 2H), 5.25 (s, 1H), 5.01 (t, J=5.3 Hz, 1H), 4.14 (d, J=10.8 Hz, 1H), 3.80 (d, J=10.3 Hz, 1H), 3.33 (m, 2H), 1.82-1.91 (m, 4H), 1.64-1.75 (m, 4H), 1.27 (s, 3H); ESI-MS: M+H⁺399.1 m/z; RPHPLC @ 6.104 min (95%); [α]D20=+48.2. Stereochemistry is arbitrarily assigned.

### Example 57

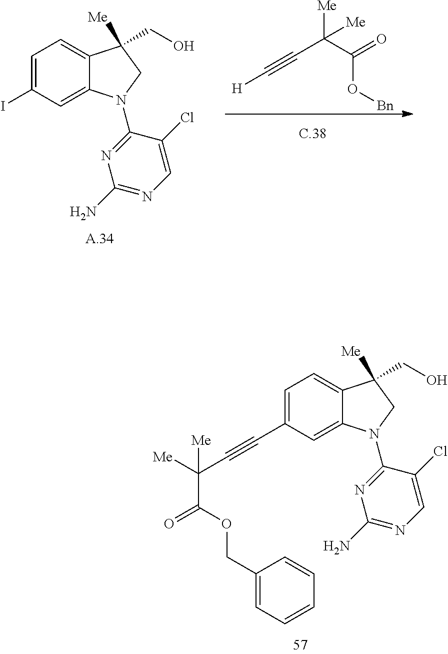

### benzyl 4-((3S*)-1-(2-amino-5-chloro-4-pyrimidinyl)-3-(hydroxymethyl)-3-methyl-2,3-dihydro-1H-indol-6-yl)-2,2-dimethyl-3-butynoate (57)

benzyl 2,2-dimethyl-3-butynoate C.38 (available from Beta Pharma Inc.) (267 mg, 1.32 mmol), and (1-(2-amino-5-chloro-4-pyrimidinyl)-6-iodo-3-methyl-2,3-dihydro-1H-indol-3-yl)methanol A.34 (138 mg, 0.33 mmol) were combined in 3 ml of DMF and 1.5 ml of triethylamine. Argon was bubbled through the solution for ˜1 min before adding PdCl₂(PPh₃)₂ (50 mg, 0.067 mmol). The reaction was heated overnight at 30° C. More benzyl 2,2-dimethyl-3-butynoate (267 mg, 1.32 mmol) and PdCl₂(PPh₃)₂ (50 mg, 0.067 mmol) were added. The solution was heated at 40° C. for 1 day before it was diluted with water and extracted with ethyl acetate. The organics were washed with Brine and dried over Na₂SO₄ before being concentrated under vacuum. The residue obtained was purified by CombiFlash chromatography eluting with a gradient of 1% methanol/dichloromethane to 2% methanol/dichloromethane. The fractions containing the product were combined and concentrated under vacuum to give benzyl 4-((3S*)-1-(2-amino-5-chloro-4-pyrimidinyl)-3-(hydroxymethyl)-3-methyl-2,3-dihydro-1H-indol-6-yl)-2,2-dimethyl-3-butynoate (57) (105 mg) as a white solid.

ESI-MS: M+H⁺ 491.2 m/z, Stereochemistry is arbitrarily assigned.

### Example 58

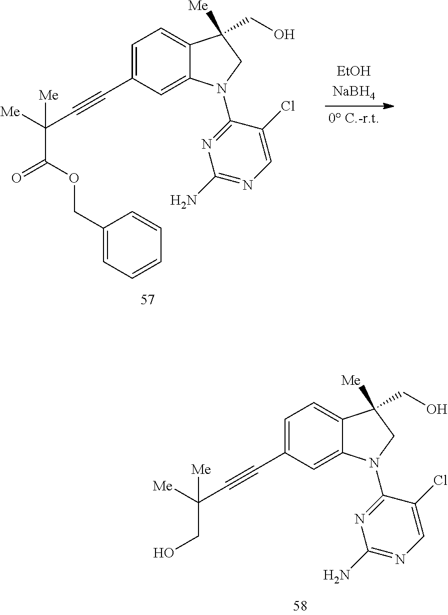

### 4-((3S*)-1-(2-amino-5-chloro-4-pyrimidinyl)-3-(hydroxymethyl)-3-methyl-2,3-dihydro-1H-indol-6-yl)-2,2-dimethyl-3-Butyn-1-ol (58)

benzyl 4-((3S*)-1-(2-amino-5-chloro-4-pyrimidinyl)-3-(hydroxymethyl)-3-methyl-2,3-dihydro-1H-indol-6-yl)-2,2-dimethyl-3-butynoate (57) (100 mg, 0.204) in absolute EtOH (5 ml) was cooled in an ice bath before adding NaBH₄ (77 mg, 2.03 mmol). The solution was then allowed to warm to room temperature. After stirring the reaction at room temperature for 3 days it was cooled in an ice bath and quenched with saturated NH₄Cl. Diluted the solution with water and extracted with ethyl acetate. The organics were then washed with Brine, dried over Na₂SO₄ and concentrated under vacuum. The residue obtained was purified by CombiFlash chromatography eluting with a gradient of 2.5% methanol/dichloromethane to 5% methanol/dichloromethane. The fractions containing the product were combined and concentrated under vacuum to give 4-((3S*)-1-(2-amino-5-chloro-4-pyrimidinyl)-3-(hydroxymethyl)-3-methyl-2,3-dihydro-1H-indol-6-yl)-2,2-dimethyl-3-butyn-1-ol (58) (25 mg, 31%).

¹H NMR (400 MHz, DMSO-d6) δ 8.09 (s, 1H), 7.16 (s, 1H), 7.13 (d, J=7.7 Hz, 1H), 6.91 (br d, J=7.6 Hz, 1H), 6.63 (br s, 2H), 4.99 (t, J=4.9 Hz, 1H), 4.91 (t, J=5.8 Hz, 1H), 4.14 (d, J=10.3 Hz, 1H), 3.79 (d, J=10.3 Hz, 2H), 3.33 (m, 4H), 1.27 (s, 3H), 1.18 (s, 6H); ESI-MS: M+H⁺387.2 m/z; RPHPLC @ 5.885 min (95%). Stereochemistry is arbitrarily assigned.

### Example 59

Examples 59-80 were prepared using same or analogous synthetic techniques and substituting with appropriate reagent as in example 1.

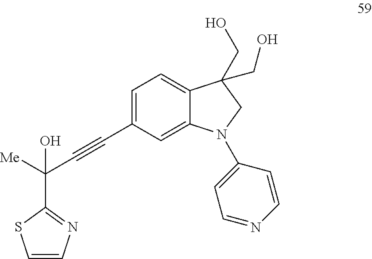

4-(3,3-bis(hydroxymethyl)-1-(4-pyridinyl)-2,3-dihydro-1H-indol-6-yl)-2-(1,3-thiazol-2-yl)-3-butyn-2-ol (59) (prepared from components A.38 and C.4). ¹H NMR (500 MHz, DMSO-d6) δ 8.39 (br m, 2H), 7.78 (d, J=3.18 Hz, 1H), 7.68 (d, J=3.48 Hz, 1H), 7.28 (d, J=7.8 Hz, 1H), 7.26 (d, J=1 Hz, 1H), 7.16 (br m, 2H), 7.00 (s, 1H), 6.95 (dd, J=7.6 Hz, J=1.25 Hz, 1H), 4.93 (s, 2H), 3.88 (s, 2H), 3.58 (m, 4H), 1.88 (s, 3H); ESI-MS: M+H⁺ 408.1 m/z; RPHPLC @ 4.559 min (93%).

### Example 60

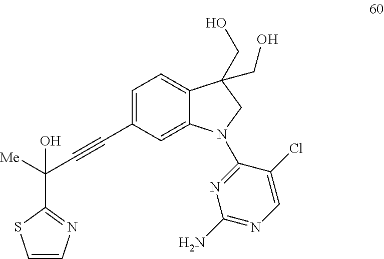

4-(1-(2-amino-5-chloro-4-pyrimidinyl)-3,3-bis(hydroxymethyl)-2,3-dihydro-1H-indol-6-yl)-2-(1,3-thiazol-2-yl)-3-butyn-2-ol (60) (prepared from components A.39 and C.4). ¹H NMR (400 MHz, DMSO-d6) δ 8.10 (s, 1H), 7.75 (d, J=3.2 Hz, 1H), 7.66 (d, J=3.2 Hz, 1H), 7.24 (d, J=1 Hz, 1H), 7.22 (d, J=7.7 Hz, 1H), 6.94 (dd, J=7.6 Hz, J=1.3, 1H), 6.93 (s, 1H), 6.61 (br s, 2H), 4.88 (t, J=5.2, 2H), 4.05 (s, 2H), 3.50-3.58 (m, 4H), 1.86 (s, 3H); ESI-MS: M+H⁺ 458.0 m/z; RPHPLC @ 4.785 min (95%).

### Example 61

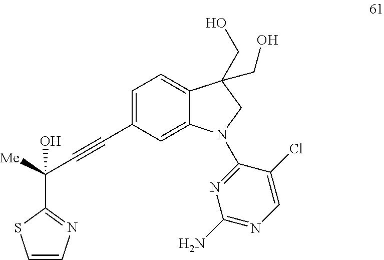

(2R)-4-(1-(2-amino-5-chloro-4-pyrimidinyl)-3,3-bis(hydroxymethyl)-2,3-dihydro-1H-indol-6-yl)-2-(1,3-thiazol-2-yl)-3-Butyn-2-ol (61) Separated the racemic mixture of example 60 on a preparative chiral column AD-H eluting with 17% iPrOH/hexane, the title compound was obtained a clear film (9 mg, 2%). Stereochemistry for the second peak to elute assigned as R based on biological activity.

¹H NMR (500 MHz, methanol-d₄) δ 8.04 (br s, 1H), 7.78 (d, J=3.4 Hz, 1H), 7.65 (br m, 1H), 7.56 (d, J=3.4 Hz, 1H), 7.27 (d, J=7.8 Hz, 1H), 7.10 (dd, J=7.6 Hz, J=1.2, 1H), 4.26 (s, 2H), 3.75 (m, 4H), 3.37 (s, 1H), 1.95 (s, 3H); ESI-MS: M+H⁺ 458.0 m/z; RPHPLC @ 4.822 min (95%).

### Example 62

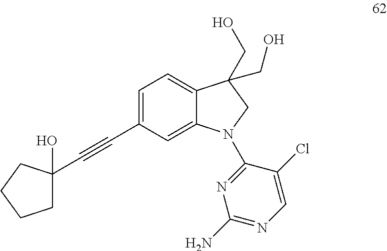

1-((1-(2-amino-5-chloro-4-pyrimidinyl)-3,3-bis(hydroxymethyl)-2,3-dihydro-1H-indol-6-yl)ethynyl)cyclopentanol (62). The title compound prepared from components A.39 and C.36 was obtained as a brownish solid (100 mg, 77%).

¹H NMR (400 MHz, DMSO-d6) δ 8.12 (br s, 1H), 7.23 (d, J=1 Hz, 1H), 7.20 (d, J=7.6 Hz, 1H), 6.92 (dd, J=7.6 Hz, J=1.3 Hz, 1H), 6.61 (br s, 2H), 5.24 (s, 1H), 4.87 (t, J=5.2 Hz, 2H), 4.05 (s, 1H), 3.50-3.58 (m, 4H), 1.82-1.92 (m, 4H), 1.64-1.74 (m, 4H); ESI-MS: M+H⁺ 415.1 m/z; RPHPLC @ 5.138 min (95%).

### Example 63

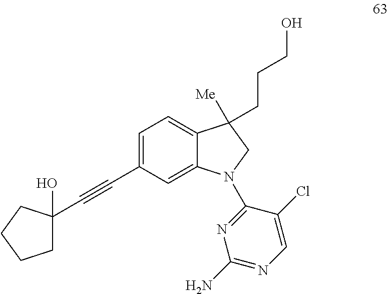

1-((1-(2-amino-5-chloro-4-pyrimidinyl)-3-(3-hydroxypropyl)-3-methyl-2,3-dihydro-1H-indol-6-yl)ethynyl)cyclopentanol (63) (prepared from components A.43 and C.36). ¹H NMR (500 MHz, DMSO-d6) δ 8.11 (s, 1H), 7.15 (m, 2H), 6.96 (d, J=7.7 Hz, 1H), 6.67 (s, 1H), 5.28 (s, 1H), 4.36 (t, J=5.1 Hz, 1H), 3.96 (d, J=10.2 Hz, 1H), 3.88 (d, J=10.3 Hz, 1H), 3.32 (m, 2H), 1.85-1.92 (m, 4H), 1.65-1.77 (m, 4H), 1.64-1.52 (m, 2H), 1.42-1.35 (m, 1H), 1.29 (s, 3H) 1.26-1.20 (m, 1H); ESI-MS: M+H⁺ 427.3 m/z; RPHPLC @ 6.254 min (95%).

### Example 64

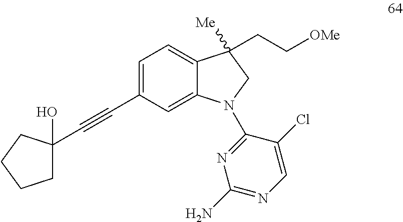

1-((1-(2-amino-5-chloro-4-pyrimidinyl)-3-(2-methoxyethyl)-3-methyl-2,3-dihydro-1H-indol-6-yl)ethynyl)cyclopentanol (64) prepared from components A.48 and C.36). ¹H NMR (500 MHz, CDCl₃) δ 8.04 (br s, 1H), 7.56 (s, 1H), 7.11 (d, J=7.8 Hz, 1H), 7.06 (d, J=7.9 Hz, 1H), 5.31 (br s, 2H), 4.30 (d, J=10.8 Hz, 1H), 3.99 (d, J=10.2 Hz, 1H), 3.26-3.35 (m, 2 H), 1.85-2.11 (m, 8H), 1.76-1.82 (m, 2H), 1.36 (s, 3H); ESI-MS: M+H⁺ 427.1 m/z; RPHPLC @ 7.083 min (94%).

### Example 65

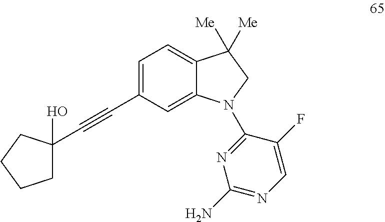

1-((1-(2-amino-5-fluoro-4-pyrimidinyl)-3,3-dimethyl-2,3-dihydro-1H-indol-6-yl)ethynyl)cyclopentanol (65) prepared from components A.54 and C.36). ¹H NMR (500 MHz, (CD)₃SO) δ 8.02 (d, J=5.4 Hz, 1H), 7.83 (br s, 1H), 7.21 (d, J=7.9 Hz, 1H), 7.00 (dd, J=7.8 Hz, J=1.5 Hz, 1H), 6.35 (br s, 2H), 5.24 (s, 1H), 3.94 (d, J=3.9 Hz, 2H), 1.87-1.92 (m, 4H), 1.65-1.77 (m, 4H), 1.29 (s, 6H); ESI-MS: M+H⁺ 367.5 m/z; RPHPLC @ 6.955 min (95%).

### Example 66

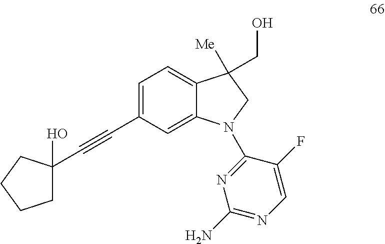

1-((1-(2-amino-5-fluoro-4-pyrimidinyl)-3-(hydroxymethyl)-3-methyl-2,3-dihydro-1H-indol-6-yl)ethynyl)cyclopentanol (66) (prepared from components A.56 and C.36). ¹H NMR (500 MHz, (CD)₃SO) δ 8.01 (d, J=5.9 Hz, 1H), 7.88 (s, 1H), 7.18 (d, J=7.8 Hz, 1H), 6.98 (dd, J=7.3 Hz, J=1.0 Hz, 1H), 6.33 (br s, 2H), 5.24 (s, 1H), 5.02 (t, J=5.4 Hz, 1H), 4.23 (dd, J=11.3 Hz, J=5.4 Hz, 1H), 3.79 (d, J=10.3 Hz, J=3.9 Hz, 1H), 3.36 (d, J=6.9 Hz, 2H), 1.88-1.92 (m, 4H), 1.65-1.76 (m, 4H), 1.28 (s, 3H); ESI-MS: M+H⁺ 383.2 m/z; RPHPLC @ 5.853 min (97%).

### Example 67

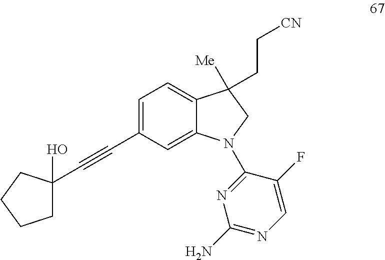

3-(1-(2-amino-5-fluoro-4-pyrimidinyl)-6-((1-hydroxycyclopentyl)ethynyl)-3-methyl-2,3-dihydro-1H-indol-3-yl)propanenitrile (67) (prepared from components A.60 and C.36). ¹H NMR (500 MHz, (CD)₃SO) δ 8.04 (br s, 1H) 7.84 (s, 1H), 7.23 (d, J=7.8 Hz, 1H), 7.01 (dd, J=7.8 Hz, J=1.5 Hz, 1H), 6.36 (br s, 2H), 5.25 (s, 1H), 4.14 (dd, J=10.8 Hz, J=4.4 Hz, 1H), 3.89 (dd, J=11.2 Hz, J=3.9 Hz, 1H), 2.41-2.48 (m, 1H), 2.23-2.30 (m, 1H), 1.91-1.99 (m, 4H), 1.90 (m, 2H), 1.65-1.76 (m, 4H); ESI-MS: M+H⁺ 406.1 m/z; RPHPLC @ 6.502 min (97%).

### Example 68

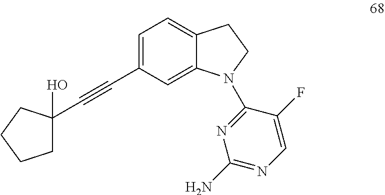

1-((1-(2-amino-5-fluoro-4-pyrimidinyl)-2,3-dihydro-1H-indol-6-yl)ethynyl)cyclopentanol (68). The title compound prepared from components A.61 and C.36, was obtained as a light brownish solid (8 mg, 3%).

¹H NMR (500 MHz, (CD)₃SO) δ 8.01 (s, 1H), 7.89 (br s, 1H), 7.20 (d, J=7.9 Hz, 1H), 6.95 (dd, J=7.8 Hz, J=1.5 Hz, 1H), 6.34 (br s, 2H), 5.24 (s, 1H), 4.18-4.23 (m, 2H), 3.14 (t, J=8.3 Hz, 2H), 1.88-1.92 (m, 4H), 1.65-1.76 (m, 4H); ESI-MS: M+H⁺ 339.4 m/z; RPHPLC @ 6.268 min (95%).

### Example 69

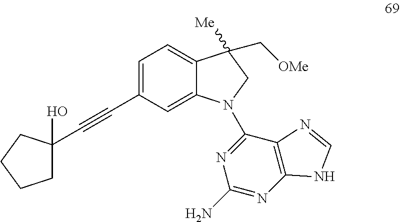

1-((1-(2-amino-9H-purin-6-yl)-3-(methoxymethyl)-3-methyl-2,3-dihydro-1H-indol-6-yl)ethynyl)cyclopentanol (69) (prepared from components A.70 and C.36). ¹H NMR (500 MHz, (CD)₃SO) δ 12.36 (br s, 1H), 8.56 (br s, 1H), 7.80 (s, 1H), 7.21 (d, J=7.8 Hz, 1H), 6.98 (br d, J=7.4 Hz, 1H), 6.03 (br s, 2H), 5.24 (s, 1H), 4.75 (d, J=11.7 Hz, 1H), 4.30 (d, J=11.7 Hz, 1H), 3.38 (m, 2H), 3.24 (s, 3H), 1.90-1.95 (m, 4H), 1.66-1.77 (m, 4H), 1.33 (s, 3H); ESI-MS: M+H⁺ 419.2 m/z; RPHPLC @ 6.418 min (95%).

### Example 70

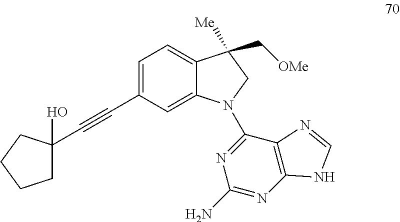

1-(((3R*)-1-(2-amino-9H-purin-6-yl)-3-(methoxymethyl)-3-methyl-2,3-dihydro-1H-indol-6-yl)ethynyl)cyclopentanol (70) The racemic mixture was separated on a preparative chiral column AD-H eluting with 30% iPrOH/hexane; the title compound was obtained as an off white solid (13 mg, 7%). Arbitrarily assigned stereochemistry as R*for the second peak to elute from the chiral column.

¹H NMR (500 MHz, (CD)₃SO) δ 12.37 (br s, 1H), 8.56 (d, J=1.4 Hz, 1H), 7.80 (s, 1H), 7.22 (d, J=7.4 Hz, 1H), 6.99 (dd, J=7.8 Hz, J=1.5 Hz, 1H), 6.03 (br s, 2H), 5.25 (s, 1H), 4.76 (d, J=11.8 Hz, 1H), 4.31 (d, J=11.8 Hz, 1H), 3.39 (m, 2H), 3.25 (s, 3H), 1.91-1.94 (m, 4H), 1.67-1.79 (m, 4H), 1.34 (s, 3H); ESI-MS: M+H⁺ 419.2 m/z; RPHPLC @ 6.412 min (95%).

[α]D20=−55°.

### Example 71

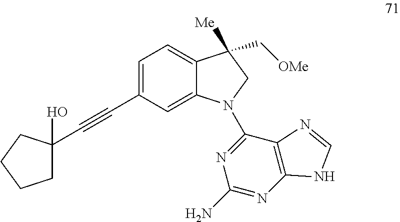

1-(((3S*)-1-(2-amino-9H-purin-6-yl)-3-(methoxymethyl)-3-methyl-2,3-dihydro-1H-indol-6-yl)ethynyl)cyclopentanol (71); The title compound (arbitrarily assigned stereochemistry as S*) was the first peak to elute from the chiral column and was obtained as an off white solid (25 mg, 15%).

¹H NMR (500 MHz, (CD)₃SO) δ 12.36 (br s, 1H), 8.56 (d, J=1.0 Hz, 1H), 7.80 (s, 1H), 7.22 (d, J=7.9 Hz, 1H), 6.99 (dd, J=7.8 Hz, J=1.5 Hz, 1H), 6.04 (br s, 2H), 5.25 (s, 1H), 4.76 (d, J=11.7 Hz, 1H), 4.31 (d, J=11.8 Hz, 1H), 3.39 (m, 2H), 3.25 (s, 3H), 1.91-1.95 (m, 4H), 1.67-1.78 (m, 4H), 1.34 (s, 3H); ESI-MS: M+ H⁺ 419.2 m/z; RPHPLC @ 6.425 min (95%).

[α]D20=+55°.

### Example 72

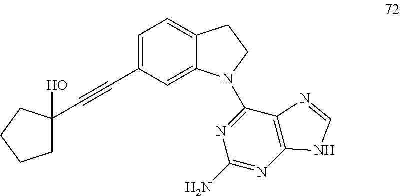

1-((1-(2-amino-9H-purin-6-yl)-2,3-dihydro-1H-indol-6-yl)ethynyl)cyclopentanol (72) (prepared from components A.72 and C.36). ¹H NMR (500 MHz, (CD)₃SO) δ 12.35 (s, 1H), 8.57 (d, J=1 Hz, 1H), 7.81 (s, 1H), 7.20 (d, J=7.4 Hz, 1H), 6.95 (dd, J=7.9 Hz, J=1.4, 1H), 6.02 (br s, 2H), 5.24 (s, 1H), 4.71 (t, J=8.3 Hz, 2H), 3.20 (t, J=8.3 Hz, 2H), 1.90-1.95 (m, 4H), 1.66-1.77 (m, 4H); ESI-MS: M+H⁺ 361.1 m/z; RPHPLC @ 6.044 min (95%).

### Example 73

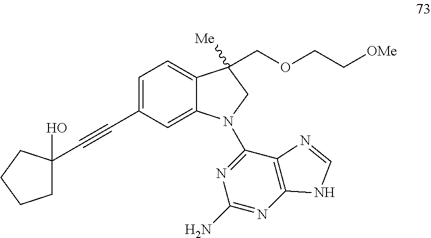

1-((1-(2-amino-9H-purin-6-yl)-3-((2-methoxyethoxy)methyl)-3-methyl-2,3-dihydro-1H-indol-6-yl)ethynyl)cyclopentanol (73) (prepared from components A.76 and C.36). ¹H NMR (500 MHz, (CD)₃SO) δ 12.37 (br s, 1H), 8.55 (br s, 1H), 7.80 (br s, 1H), 7.22 (d, J=7.3 Hz, 1H), 6.98 (br d, J=7.9 Hz, 1H), 6.04 (br s, 2H), 5.24 (br s, 1H), 4.74 (d, J=11.8 Hz, 1H), 4.31 (d, J=11.7 Hz, 1H), 3.50 (t, J=5.4 Hz, 2H), 3.47 (s, 2H), 3.40 (t, 4.4 Hz, 2H), 3.18 (s, 3H), 1.90-1.95 (m, 4H), 1.65-1.77 (m, 4H), 1.34 (s, 3H); ESI-MS: M+H⁺463.5 m/z; RPHPLC @ 6.005 min (95%).

### Example 74

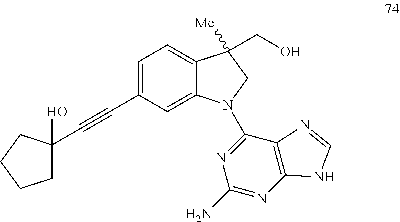

1-((1-(2-amino-9H-purin-6-yl)-3-(hydroxymethyl)-3-methyl-2,3-dihydro-1H-indol-6-yl)ethynyl)cyclopentanol (74) (prepared from components A.77 and C.36).¹H NMR (500 MHz, (CD)₃SO) δ 12.35 (br s, 1H), 8.54 (d, J=1 Hz, 1H), 7.80 (s, 1H), 7.18 (d, J=7.9 Hz, 1H), 6.98 (dd, J=7.9 Hz, J=1.5 Hz, 1H), 6.02 (br s, 2H), 5.24 (br s, 1H), 5.02 (t, J=5.4 Hz, 1H), 4.80 (d, J=11.7 Hz, 1H), 4.24 (d, J=11.8 Hz, 1H), 1.90-1.94 (m, 4H), 1.65-1.77 (m, 4H), 1.31 (s, 3H); ESI-MS: M+H⁺405.4 m/z; RPHPLC @ 5.623 min (95%).

### Example 75

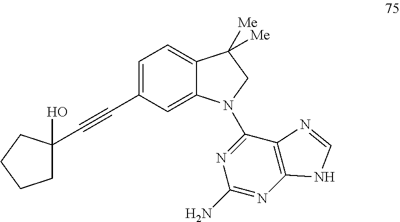

1-((1-(2-amino-9H-purin-6-yl)-3,3-dimethyl-2,3-dihydro-1H-indol-6-yl)ethynyl)cyclopentanol (75). The title compound prepared from components A.78 and C.36 was obtained as a white solid (70 mg, 44%).

¹H NMR (400 MHz, (CD)₃SO) δ 12.34 (s, 1H), 8.53 (d, J=1.3 Hz, 1H), 7.80 (s, 1H), 7.21 (d, J=7.6 Hz, 1H), 7.00 (dd, J=7.6 Hz, J=1.4 Hz, 1H), 6.03 (br s, 2H), 5.23 (s, 1H), 4.48 (s, 2 H), 1.88-1.93 (m, 4H), 1.66-1.77 (m, 4H), 1.33 (s, 6H); ESI-MS: M+H⁺ 389.5 m/z; RPHPLC @ 6.495 min (95%).

### Example 76

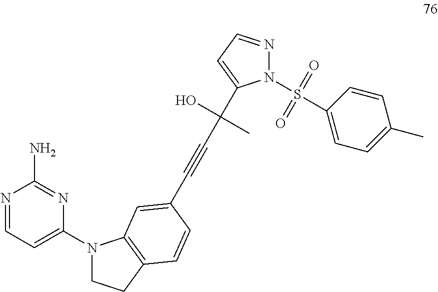

4-(1-(2-Amino-4-pyrimidinyl)-2,3-dihydro-1H-indol-6-yl)-2-(1-((4-methylphenyl)sulfonyl)-1H-pyrazol-5-yl)-3-butyn-2-ol (76) was prepared from components A.95 and C.30. ¹H NMR (500 MHz, DMSO-d₆) δ ppm 8.40 (2H, s), 7.99 (1H, s), 7.88 (2H, d, J=7.8 Hz), 7.46 (2H, d, J=7.6 Hz), 7.19 (1H, s), 6.89 (1H, d, J=7.6 Hz), 6.73 (1H, s), 6.33 (2H, s), 6.29 (1H, s), 6.04 (1H, s), 3.93-4.01 (2H, m), 3.16 (2H, s), 2.36 (3H, s), 1.75 (3H, s). MS ESI (pos.) m/e: 501.2 (M+H).

### Example 77

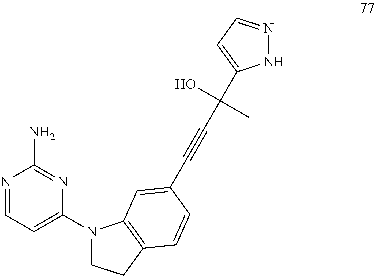

4-(1-(2-Amino-4-pyrimidinyl)-2,3-dihydro-1H-indol-6-yl)-2-(1H-pyrazol-5-yl)-3-butyn-2-ol (77). To a flask containing 4-(1-(2-amino-4-pyrimidinyl)-2,3-dihydro-1H-indol-6-yl)-2-(1-((4-methylphenyl)sulfonyl)-1H-pyrazol-5-yl)-3-butyn-2-ol (76) (21.7 mg, 0.043 mmol) in methanol (3.00 mL) was added 5 M NaOH (1.00 mL). This mixture was stirred at room temperature for 45 minutes then diluted with water. After extracting three times with ethyl acetate, the organic solvent was removed under reduced pressure. The residue was purified by silica gel flash chromatography (0-100% methanol in dichloromethane) afford a mixture of tautomers of 4-(1-(2-amino-4-pyrimidinyl)-2,3-dihydro-1H-indol-6-yl)-2-(1H-pyrazol-5-yl)-3-butyn-2-ol and 4-(1-(2-amino-4-pyrimidinyl)-2,3-dihydro-1H-indol-6-yl)-2-(1H-pyrazol-3-yl)-3-butyn-2-ol (10.2 mg, 68%). ¹H NMR (400 MHz, DMSO-d₆) δ ppm 12.55 (1 H, m), 8.39 (1H, m), 7.98 (1H, m), 7.63 (1H, m), 7.18 (1H, d, J=7.4 Hz), 6.94 (1H, m), 6.29 (2H, m), 6.04 (1H, d, J=5.9 Hz), 3.97 (2H, t, J=8.6 Hz), 3.16 (3H, t, J=8.4 Hz), 1.81 (3H, s). MS ESI (pos.) m/e: 347.2 (M+H).

Examples 78-80 were prepared using same or analogous synthetic techniques and substituting with appropriate reagent as in example 77.

### Example 78

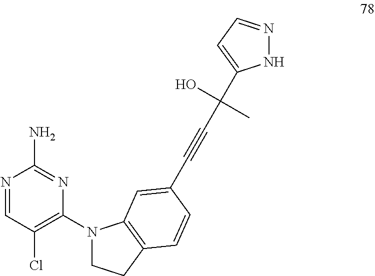

4-(1-(2-Amino-5-chloro-4-pyrimidinyl)-2,3-dihydro-1H-indol-6-yl)-2-(1H-pyrazol-5-yl)-3-butyn-2-ol (78). (prepared from components A.2 and C.30).

¹H NMR (500 MHz, DMSO-d₆) δ ppm 12.58 (1H, m), 8.11 (1H, m), 7.21 (1H, d, J=7.6 Hz), 7.15 (1H, m), 6.94 (1H, d, J=6.8 Hz), 6.65 (2H, s), 6.31 (1H, s), 4.16 (2H, t, J=8.2 Hz), 3.09 (2H, t, J=8.2 Hz), 1.77 (3H, s). MS ESI (pos.) m/e: 381.1 (M+H).

### Example 79

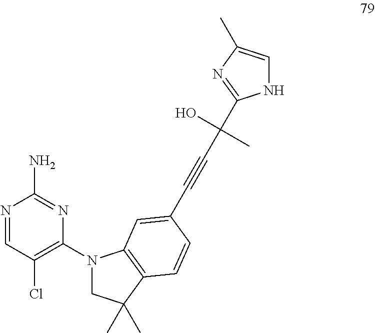

4-(1-(2-Amino-5-chloro-4-pyrimidinyl)-3,3-dimethyl-2,3-dihydro-1H-indol-6-yl)-2-(4-methyl-1H-imidazol-2-yl)-3-butyn-2-ol (79) (prepared from components A.15 and C.27). MS ESI (pos.) m/e: 423.1 (M+H).

### Example 80

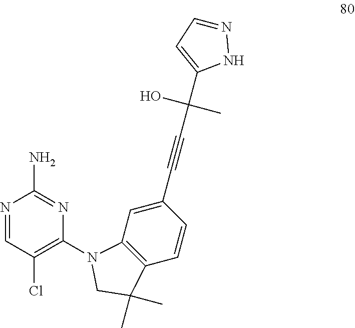

4-(1-(2-amino-5-chloropyrimidin-4-yl)-3,3-dimethylindolin-6-yl)-2-(1H-pyrazol-5-yl)but-3-yn-2-ol and 4-(1-(2-amino-5-chloropyrimidin-4-yl)-3,3-dimethylindolin-6-yl)-2-(1H-pyrazol-3-yl)but-3-yn-2-ol (80) (prepared from components A.15 and C.30). MS ESI (pos.) m/e: 409.1 (M+H).

### Example 81 and 82

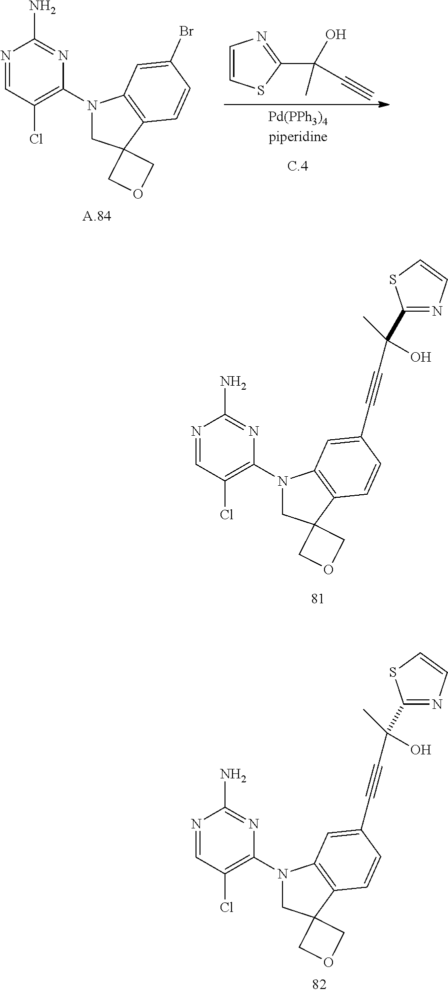

To a flask containing bromo oxetane A.84 (191.2 mg, 0.52 mmol), 2-(thiazol-2-yl)but-3-yn-2-ol C.4 (319.0 mg, 2.08 mmol), and Pd(PPh₃)₄ (90.0 mg, 0.078 mmol) was added piperidine (5.0 mL). After heating at 80° C. for 20 hours, the organic solvent was removed under reduced pressure. The residue was purified by silica gel flash chromatography (45:1:1 solution of dichloromethane: methanol: 2M ammonia in methanol) to afford mixture of enantiomers. The enantiomers were separated with Chiral HPLC using an AD-H column with isocratic 17% isopropanol in hexanes. The first eluted enantiomer (81) was a brown solid; the second eluted enantiomer (82) also a brown solid was assigned as (R)-4-(1-(2-amino-5-chloropyrimidin-4-yl)spiro[indoline-3,3′-oxetane]-6-yl)-2-(thiazol-2-yl)but-3-yn-2-ol based on biological activity: ¹H NMR (500 MHz, methanol-d4) δ ppm 8.04 (1H, s), 7.76 (1H, d, J=3.2 Hz), 7.67 (1H, d, J=7.8 Hz), 7.63 (1H, d, J=1.2 Hz), 7.55 (1H, d, J=3.4 Hz), 7.21 (1H, dd, J=7.7, 1.3 Hz), 4.89 (4H, m), 4.61 (2H, s), 1.93 (3H, m). MS ESI (pos.) m/e: 440.0 (M+H).

Examples 83-118 were prepared using the same or analogous synthetic techniques and substituting with appropriate reagents as in example 1.

### Example 83

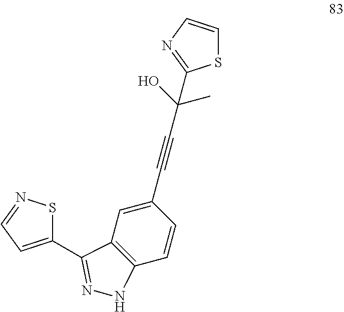

4-(3-(isothiazol-5-yl)-1H-indazol-5-yl)-2-(thiazol-2-yl)but-3-yn-2-ol (83) (prepared from components A.160 and C.4). ¹H NMR (400 MHz, DMSO-d₆) δ ppm 13.80 (1H, s), 8.66 (1H, d, J=1.6 Hz), 8.20 (1H, s), 8.14 (1H, d, J=1.2 Hz), 7.78 (1H, d, J=3.1 Hz), 7.69 (1H, d, J=3.1 Hz), 7.66 (1H, d, J=8.6 Hz), 7.47 (1H, d, J=8.6 Hz), 7.03 (1H, s), 1.92 (3H, s)

### Example 87

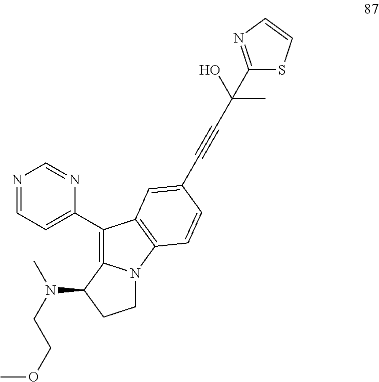

4-((R*)-1-((2-methoxyethyl)(methyl)amino)-9-(pyrimidin-4-yl)-2,3-dihydro-1H-pyrrolo[1,2-a]indol-7-yl)-2-(thiazol-2-yl)but-3-yn-2-ol (87). (Prepared from components A.150 and C.4) 1H NMR (500 MHz, DMSO-d₆) δ ppm 9.14 (1H, s), 8.79 (1H, s), 8.59 (1H, d, J=5.4 Hz), 8.29 (1H, d, J=5.4 Hz), 7.78 (1H, d, J=3.2 Hz), 7.69 (1H, d, J=3.2 Hz), 7.46 (1 H, d, J=8.3 Hz), 7.25 (1H, d, J=8.3 Hz), 7.01 (1H, s), 4.91 (1H, d, J=6.1 Hz), 4.21 (2H, t, J=8.1 Hz), 3.45 (2H, t, J=5.0 Hz), 3.25 (3H, s), 2.74 (2H, dd, J=12.6, 6.0 Hz), 2.53-2.63 (2 H, m), 2.11 (3H, s), 1.90 (3H, s)

### Example 88

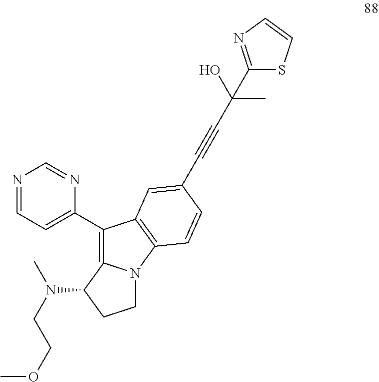

4-((S*)-1-((2-methoxyethyl)(methyl)amino)-9-(pyrimidin-4-yl)-2,3-dihydro-1H-pyrrolo[1,2-a]indol-7-yl)-2-(thiazol-2-yl)but-3-yn-2-ol (88). Prepared from components A.151 and C.4. 1H NMR (500 MHz, DMSO-d₆) δ ppm 8.92 (1H, s), 8.57 (1H, s), 8.37 (1H, d, J=5.4 Hz), 8.07 (1H, d, J=5.1 Hz), 7.56 (1H, d, J=2.7 Hz), 7.46 (1H, d, J=2.9 Hz), 7.23 (1 H, d, J=8.1 Hz), 7.03 (1H, d, J=8.3 Hz), 6.78 (1H, s), 4.69 (1H, d, J=6.6 Hz), 3.99 (2H, t, J=7.8 Hz), 3.23 (2H, s), 3.02 (3H, s), 2.48-2.55 (2H, m, J=12.0, 5.9 Hz), 2.31-2.41 (2H, m), 1.89 (3H, s), 1.68 (3H, s)

### Example 89

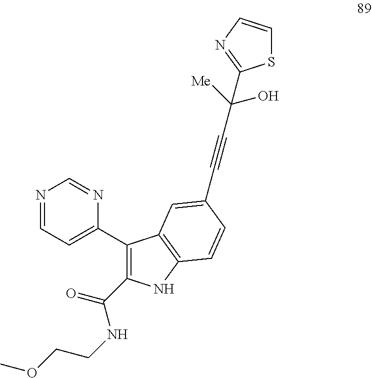

5-(3-hydroxy-3-(thiazol-2-yl)but-1-ynyl)-N-(2-methoxyethyl)-3-(pyrimidin-4-yl)-1H-indole-2-carboxamide (89). (prepared from components A.161 and C.4). ¹H NMR (400 MHz, DMSO-d₆) δ ppm 10.36 (1H, s), 9.24 (1H, s), 8.85 (1H, d, J=5.5 Hz), 8.13 (1H, s), 7.88 (1 H, d, J=5.5 Hz), 7.77 (1H, d, J=3.1 Hz), 7.68 (1H, d, J=3.1 Hz), 7.55 (1H, d, J=8.2 Hz), 7.33 (1H, d, J=8.6 Hz), 7.00 (1H, s), 3.51 (4H, s), 1.89 (3H, s)

### Example 90

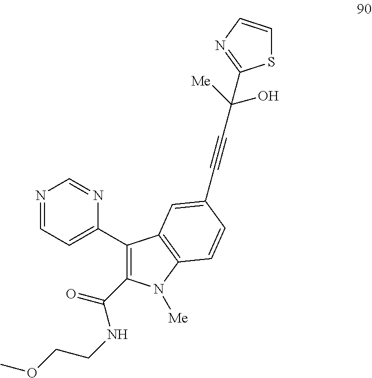

5-(3-hydroxy-3-(thiazol-2-yl)but-1-ynyl)-N-(2-methoxyethyl)-1-methyl-3-(pyrimidin-4-yl)-1H-indole-2-carboxamide (90) (prepared from components A.162 and C.4). 1H NMR (500 MHz, DMSO-d₆) δ ppm 9.23 (1H, s), 9.15 (1H, s), 8.71 (1H, d, J=5.4 Hz), 8.55 (1H, s), 7.79 (1H, d, J=3.2 Hz), 7.70 (1H, d, J=3.2 Hz), 7.65 (1H, d, J=8.6 Hz), 7.61 (1H, dd, J=5.5, 1.1 Hz), 7.38 (1H, dd, J=8.6, 1.5 Hz), 7.02 (1H, s), 3.80 (3H, s), 3.51 (4H, s), 3.29 (3H, s), 1.91 (3H, s)

### Example 91

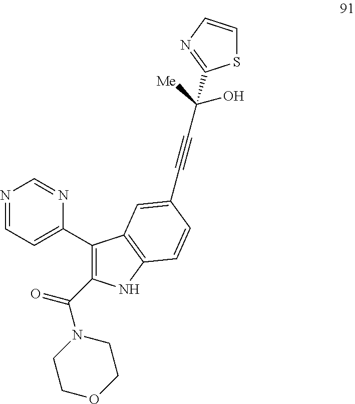

(5-((R)-3-hydroxy-3-(thiazol-2-yl)but-1-ynyl)-3-(pyrimidin-4-yl)-1H-indol-2-yl)(morpholino)methanone (91) (prepared from components A.165 and C.6). 1H NMR (500 MHz, DMSO-d₆) δ ppm 12.53 (1H, s), 9.22 (1H, s), 8.41 (1H, s), 7.78 (1H, s), 7.68 (1H, s), 7.63 (1H, s), 7.49 (1H, d, J=6.4 Hz), 7.31 (1H, d, J=6.6 Hz), 7.00 (1H, s), 3.67-3.76 (4H, m), 3.38-3.42 (2H, m), 3.12-3.21 (2H, m), 1.90 (3H, s)

### Example 92

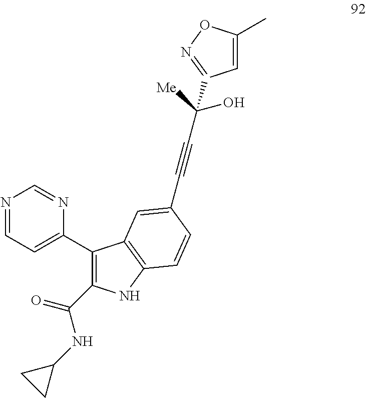

N-cyclopropyl-5-((R)-3-hydroxy-3-(4-methyl-5H-pyrrol-2-yl)but-1-ynyl)-3-(pyrimidin-4-yl)-1H-indole-2-carboxamide (92) (prepared from components A.168 and C.2). ¹H NMR (500 MHz, CHLOROFORM-d) δ ppm 11.49 (1H, s), 10.42 (1H, s), 9.11 (1H, s), 7.86 (1H, s), 7.77 (1H, d, J=5.1 Hz), 7.43 (1H, d, J=8.6 Hz), 7.33 (1H, d, J=8.6 Hz), 7.20 (3H, s), 6.12 (1H, s), 2.91-3.05 (1H, m), 2.38 (3H, s), 1.91 (3H, s), 0.82-0.88 (2H, m), 0.57-0.63 (2 H, m)

### Example 93

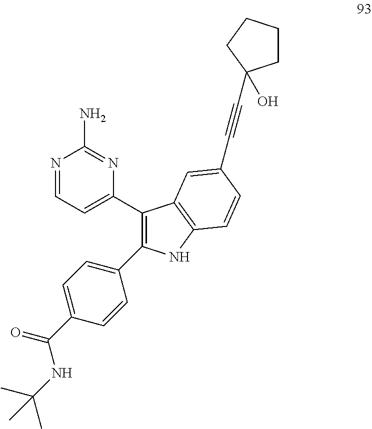

4-(3-(2-aminopyrimidin-4-yl)-5-(2-(1-hydroxycyclopentyl)ethynyl)-1H-indol-2-yl)-N-tert-butylbenzamide (93) (prepared from components A.124 and C.36). ¹H NMR (500 MHz, DMSO-d₆) δ ppm 12.00 (1H, s), 8.15 (1H, s), 8.02 (1H, d, J=5.0 Hz), 7.89 (2H, d, J=8.2 Hz), 7.85 (1H, s), 7.62 (2H, d, J=8.2 Hz), 7.42 (1H, d, J=8.2 Hz), 7.21 (1H, dd, J=8.2, 1.4 Hz), 6.58 (2H, s), 6.25 (1H, d, J=5.0 Hz), 5.23 (1H, s), 1.85-1.96 (4H, m), 1.63-1.78 (4H, m), 1.40 (9H, s); Mass Spectrum (ESI) m/e=494.2 [M+1].

### Example 94

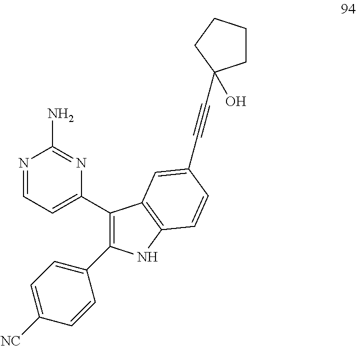

4-(3-(2-aminopyrimidin-4-yl)-5-(2-(1-hydroxycyclopentyl)ethynyl)-1H-indol-2-yl)benzonitrile (94) (prepared from components A.125 and C.36). ¹H NMR (500 MHz, DMSO-d₆) δ ppm 12.12 (1H, s), 8.09 (1H, d, J=5.0 Hz), 8.05 (1H, s), 7.93 (2H, d, J=8.2 Hz), 7.76 (2H, d, J=8.2 Hz), 7.44 (1H, d, J=8.2 Hz), 7.23 (1H, dd, J=8.5, 1.1 Hz), 6.59 (2H, s), 6.33 (1H, d, J=5.5 Hz), 5.24 (1H, s), 1.84-1.97 (4H, m), 1.62-1.80 (4H, m); Mass Spectrum (ESI) m/e=420.1 [M+1].

### Example 95

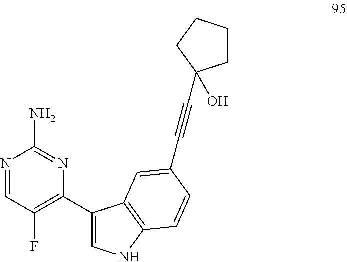

1-(2-(3-(2-amino-5-fluoropyrimidin-4-yl)-1H-indol-5-yl)ethynyl)cyclopentanol (95). (prepared from components A.129 and C.36). ¹H NMR (400 MHz, DMSO-d₆) δ ppm 11.96 (1 H, s), 8.67 (1H, s), 8.18 (1H, d, J=4.0 Hz), 8.10 (1H, d, J=2.6 Hz), 7.45 (1H, d, J=8.3 Hz), 7.22 (1H, dd, J=8.4, 1.5 Hz), 6.50 (2H, s), 5.21 (1H, s), 1.93 (4H, m), 1.72 (4H, m); Mass Spectrum (ESI) m/e=337.1 [M+1].

### Example 96

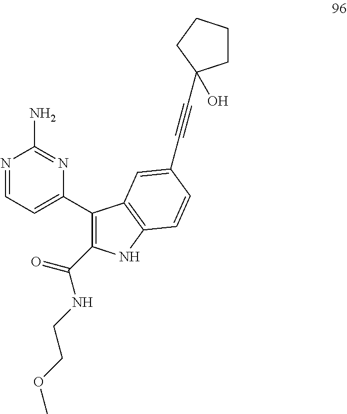

3-(2-aminopyrimidin-4-yl)-5-(2-(1-hydroxycyclopentyl)ethynyl)-N-(2-methoxyethyl)-1H-indole-2-carboxamide (96) (prepared from components A.133 and C.36). ¹H NMR (400 MHz, DMSO-d₆) δ ppm 12.29 (1H, s), 11.21 (1H, t, J=5.1 Hz), 8.41 (1H, d, J=5.1 Hz), 7.88 (1H, s), 7.53 (1H, d, J=8.4 Hz), 7.29 (1H, dd, J=8.4, 1.5 Hz), 7.00 (1H, d, J=5.1 Hz), 6.84 (2 H, s), 5.25 (1H, s), 3.50-3.62 (4H, m), 3.28 (3H, s), 1.83-1.96 (4H, m), 1.62-1.80 (4H, m); Mass Spectrum (ESI) m/e=420.1 [M+1].

### Example 97

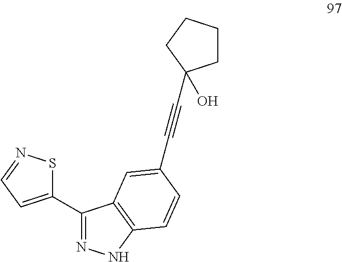

1-(2-(3-(isothiazol-5-yl)-1H-indazol-5-yl)ethynyl)cyclopentanol (97) (prepared from components A.160 and C.36).

¹H NMR (400 MHz, DMSO-d₆) δ ppm 13.76 (1H, s), 8.66 (1H, d, J=1.8 Hz), 8.16 (1H, s), 8.13 (1H, d, J=1.8 Hz), 7.64 (1H, d, J=8.4 Hz), 7.45 (1H, dd, J=8.6, 1.3 Hz), 5.31 (1H, s), 1.88-1.97 (4H, m), 1.63-1.82 (4H, m); Mass Spectrum (ESI) m/e=310.3 [M+1].

### Example 98

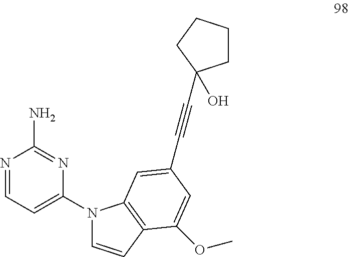

1-(2-(1-(2-aminopyrimidin-4-yl)-4-methoxy-1H-indol-6-yl)ethynyl)cyclopentanol (98) (prepared from components A.134 and C.36). ¹H NMR (400 MHz, DMSO-d₆) δ ppm 8.25-8.31 (2H, m), 7.97 (1H, d, J=3.7 Hz), 6.89-6.97 (3H, m), 6.75 (1H, d, J=3.7 Hz), 6.70 (1H, s), 5.25 (1H, s), 3.91 (3H, s), 1.95 (4H, t, J=5.9 Hz), 1.65-1.85 (4H, m); Mass Spectrum (ESI) m/e=349.1 [M+1].

### Example 99

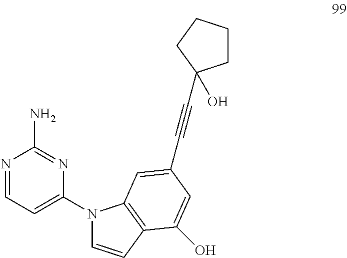

1-(2-aminopyrimidin-4-yl)-6-(2-(1-hydroxycyclopentyl)ethynyl)-1H-indol-4-ol (99) (prepared from components A.135 and C.36). ¹H NMR (400 MHz, DMSO-d₆) δ ppm 9.87 (1 H, s), 8.28 (1H, s), 8.11 (1H, s), 7.91 (1H, d, J=3.7 Hz), 6.89 (3H, d, J=5.5 Hz), 6.78 (1H, d, J=3.7 Hz), 6.58 (1H, d, J=1.1 Hz), 5.24 (1H, s), 1.88-1.96 (4H, m), 1.64-1.80 (4H, m); Mass Spectrum (ESI) m/e=335.1 [M+1].

### Example 100

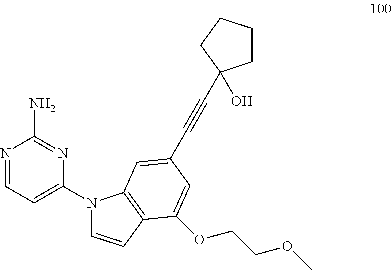

1-(2-(1-(2-aminopyrimidin-4-yl)-4-(2-methoxyethoxy)-1H-indol-6-yl)ethynyl)cyclopentanol (100) (prepared from components A.136 and C.36). ¹H NMR (400 MHz, DMSO-d₆) δ ppm 8.24-8.31 (2H, m), 7.97 (1H, d, J=4.0 Hz), 6.89-6.96 (3H, m), 6.72-6.75 (1H, m), 6.71 (1H, d, J=0.7 Hz), 5.25 (1H, s), 4.25 (2H, dd, J=5.5, 3.7 Hz), 3.70-3.77 (2H, m), 3.36 (3H, s), 1.89-1.97 (4H, m), 1.63-1.81 (4H, m); Mass Spectrum (ESI) m/e=393.1 [M+1].

### Example 101

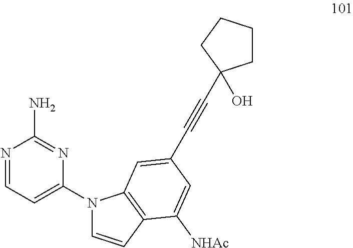

N-(1-(2-aminopyrimidin-4-yl)-6-(2-(1-hydroxycyclopentyl)ethynyl)-1H-indol-4-yl)acetamide (101) (prepared from components A.139 and C.36). ¹H NMR (500 MHz, DMSO-d₆) δ ppm 9.74 (1H, s), 8.40 (1H, s), 8.29 (1H, d, J=5.5 Hz), 8.05 (1H, d, J=3.7 Hz), 7.88 (1H, s), 7.09 (1H, d, J=3.7 Hz), 6.95 (2H, s), 6.91 (1H, d, J=5.5 Hz), 5.28 (1H, s), 2.17 (3H, s), 1.89-1.97 (4H, m), 1.65-1.79 (4H, m); Mass Spectrum (ESI) m/e=376.1 [M+1].

### Example 102

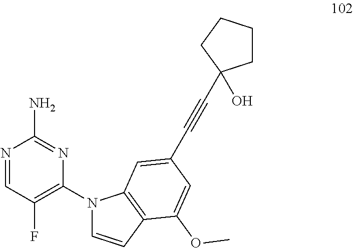

1-((1-(2-amino-5-fluoropyrimidin-4-yl)-4-methoxy-1H-indol-6-yl)ethynyl)cyclopentanol (102) (prepared from components A.88 and C.36). ¹H NMR (400 MHz, DMSO-d₆) δ ppm 8.46 (1H, d, J=4.0 Hz), 7.79 (1H, d, J=1.1 Hz), 7.70 (1H, t, J=3.3 Hz), 6.97 (2H, s), 6.77 (1 H, dd, J=3.5, 0.9 Hz), 6.71 (1H, d, J=1.1 Hz), 5.26 (1H, s), 3.92 (3H, s), 1.87-1.98 (4H, m), 1.63-1.82 (4H, m); Mass Spectrum (ESI) m/e=367.1 [M+1].

### Example 103

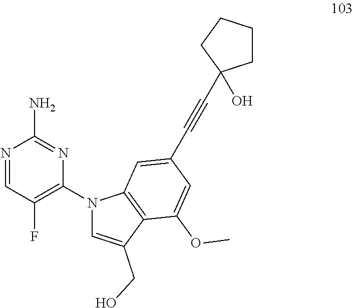

1-(2-(1-(2-amino-5-fluoropyrimidin-4-yl)-3-(hydroxymethyl)-4-methoxy-1H-indol-6-yl)ethynyl)cyclopentanol (103) (prepared from components A.142 and C.36). ¹H NMR (500 MHz, DMSO-d₆) δ ppm 8.43 (1H, d, J=3.2 Hz), 7.82 (1H, s), 7.54 (1H, s), 6.93 (2H, s), 6.67 (1H, s), 5.26 (1H, s), 5.00 (1H, t, J=5.3 Hz), 4.78 (2H, d, J=5.0 Hz), 3.88 (3H, s), 1.92 (4H, s), 1.63-1.81 (4H, m); Mass Spectrum (ESI) m/e=397.1 [M+1].

### Example 104

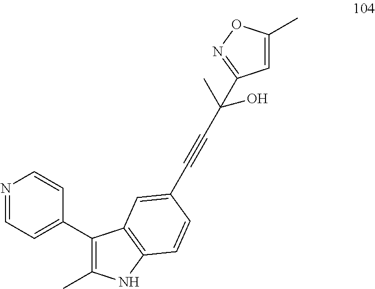

4-(2-methyl-3-(pyridin-4-yl)-1H-indol-5-yl)-2-(5-methylisoxazol-3-yl)but-3-yn-2-ol (104) (prepared from components A.143 and C.1). ¹H NMR (400 MHz, CHLOROFORM-d) δ ppm 11.65 (1H, s), 8.61 (2H, d, J=5.5 Hz), 7.65 (1H, s), 7.48 (2H, d, J=5.9 Hz), 7.37 (1H, d, J=8.6 Hz), 7.15 (1H, dd, J=8.4, 1.0 Hz), 6.35 (2H, d, J=14.5 Hz), 2.52 (3H, s), 2.40 (3H, s), 1.78 (3H, s); Mass Spectrum (ESI) m/e=358.1 [M+1].

### Example 105

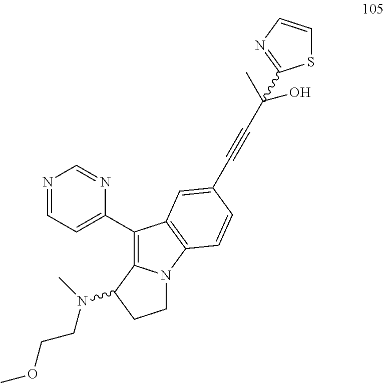

4-(1-((2-methoxyethyl)(methyl)amino)-9-(pyrimidin-4-yl)-2,3-dihydro-1H-pyrrolo[1,2-a]indol-7-yl)-2-(thiazol-2-yl)but-3-yn-2-ol (105) (prepared from components A.149 and C.4).

¹H NMR (400 MHz, DMSO-d₆) δ ppm 9.14 (1H, s), 8.79 (1H, d, J=0.8 Hz), 8.59 (1H, d, J=5.5 Hz), 8.30 (1H, dd, J=5.5, 1.2 Hz), 7.78 (1H, d, J=3.1 Hz), 7.68 (1H, d, J=3.1 Hz), 7.46 (1H, d, J=8.6 Hz), 7.25 (1H, dd, J=8.4, 1.0 Hz), 6.99 (1H, s), 4.92 (1H, dd, J=8.2, 2.7 Hz), 4.16-4.29 (2H, m), 3.46 (2H, t, J=5.5 Hz), 3.25 (3H, s), 2.69-2.81 (2H, m), 2.52-2.66 (2 H, m), 2.12 (3H, s), 1.90 (3H, s); Mass Spectrum (ESI) m/e=474.2 [M+1].

### Example 106

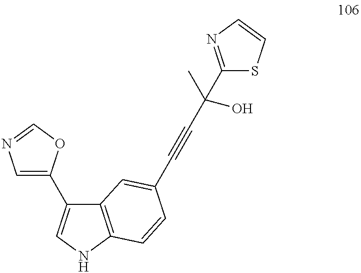

4-(3-(oxazol-5-yl)-1H-indol-5-yl)-2-(thiazol-2-yl)but-3-yn-2-ol (106) (prepared from components A.155 and C.4). ¹H NMR (400 MHz, DMSO-d₆) δ ppm 11.79 (1H, s), 8.36 (1H, s), 7.91 (1H, s), 7.88 (1H, d, J=2.7 Hz), 7.78 (1H, d, J=2.7 Hz), 7.68 (1H, d, J=3.1 Hz), 7.42-7.51 (2H, m), 7.23 (1H, d, J=8.2 Hz), 6.96 (1H, s), 1.90 (3H, s); Mass Spectrum (ESI) m/e=336.1 [M+1].

### Example 107

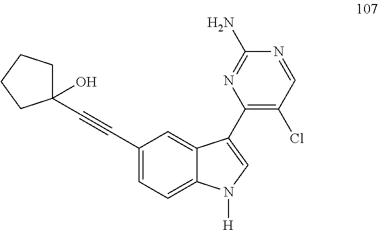

1-(2-(3-(2-amino-5-chloropyrimidin-4-yl)-1H-indol-5-yl)ethynyl)cyclopentanol (107) (prepared from components A.170 and C.36). ¹H NMR (methanol-d₄) δ 8.67 (s, 1H), 8.45 (s, 1 H), 8.21 (s, 1H), 7.43 (d, J=8.5 Hz, 1H), δ 7.29 (d, J=8.5 Hz, 1H), 2.12-2.03 (m, 4H), 1.94-1.81 (m, 4H); ms 353.4 (M+H⁺).

### Example 108

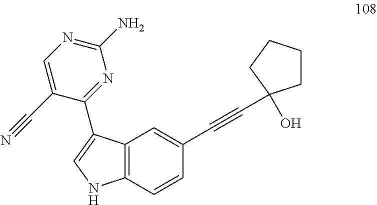

2-amino-4-(5-(2-(1-hydroxycyclopentyl)ethynyl)-1H-indol-3-yl)pyrimidine-5-carbonitrile (108) (prepared from components A.175 and C.36). ¹H NMR (methanol-d₄) δ 8.75 (s, 1H), 8.55 (s, 1H), 8.52 (s, 1H), 7.45 (d, J=8.5 Hz, 1H), δ 7.32 (d, J=8.5 Hz, 1H), 2.15-2.03 (m, 4 H), 1.95-1.78 (m, 4H); ms 344.2 (M+H⁺).

### Example 109

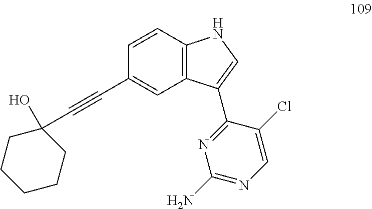

1-(2-(3-(2-amino-5-chloropyrimidin-4-yl)-1H-indol-5-yl)ethynyl)cyclohexanol (109) (prepared from components A.170 and C.9). ¹H NMR (DMSO-d₆) δ 8.50 (s, 1H), 8.39 (s, 1H), 8.27 (s, 1H), 7.45 (d, J=8.2 Hz, 1H), δ 7.21 (d, J=8.2 Hz, 1H), 6.79 (s, 2H), 5.29 (s, 1H), 1.92-1.80 (m, 2H), 1.70-1.45 (m, 7H), 1.35-1.20 (m, 1H); ms 367.1 (M+H⁺).

### Example 111

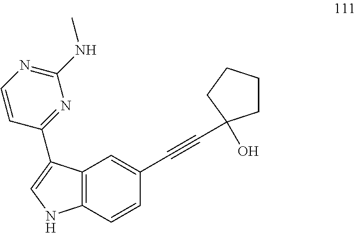

1-(2-(3-(2-(methylamino)pyrimidin-4-yl)-1H-indol-5-yl)ethynyl)cyclopentanol (111) (prepared from components A.177 and C.36). ¹H NMR (DMSO-d₆) δ 11.84 (s, 1H), 8.63 (s, 1 H), 8.28 (s, 1H), 8.16 (d, J=5.0 Hz, 1H), 7.43 (d, J=8.5 Hz, 1H), δ 7.20 (d, J=8.5 Hz, 1H), 7.01 (d, J=5.0 Hz, 1H), 6.93 (br, 1H), 5.27 (s, 1H), 2.92 (br, 1H), 2.91 (s, 3H),1.93-1.85 (m, 4 H), 1.80-1.65 (m, 4H); ms 333.1 (M+H⁺).

### Example 112

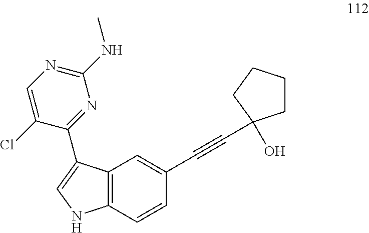

1-(2-(3-(5-chloro-2-(methylamino)pyrimidin-4-yl)-1H-indol-5-yl)ethynyl)cyclopentanol (112) (prepared from components A.172 and C.36). ¹H NMR (DMSO-d₆) δ 11.99 (s, 1H), 8.65 (br, 1H), 8.50 (s, 1H), 8.30 (s, 1H), 7.48 (d, J=8.5 Hz, 1H), 7.30 (br, 1H), 7.23 (d, J=8.5 Hz, 1H), 2.94 (br, 4H), 1.93-1.85 (m, 4H), 1.80-1.65 (m, 4H); ms 367.1 (M+H⁺).

### Example 113

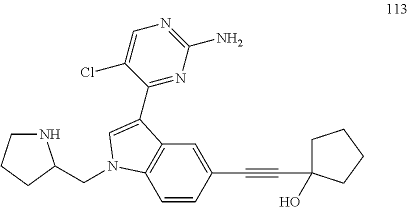

1-(2-(3-(2-amino-5-chloropyrimidin-4-yl)-1-(pyrrolidin-2-ylmethyl)-1H-indol-5-yl)ethynyl)cyclopentanol (113) (prepared from components A.178 and C.36). ¹H NMR (DMSO-d₆) δ 8.66 (s, 1H), 8.47 (s, 1H), 8.18 (s, 1H), 7.50 (d, J=8.4 Hz, 1H), 7.32 (d, J=8.4 Hz, 1H), 4.34-4.24 (m, 2H), 3.60-3.52 (m, 1H), 3.10-2.95 (m, 1H), 2.90-2.85 (m, 1H)), 2.15-1.98 (m, 4H), 1.98-1.70 (m, 7H), 1.55-1.50 (m, 1H); ms 436.1 (M+H⁺).

### Example 114

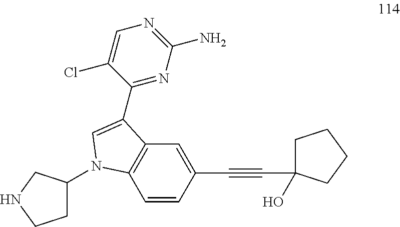

1-(2-(3-(2-amino-5-chloropyrimidin-4-yl)-1-(pyrrolidin-3-yl)-1H-indol-5-yl)ethynyl)cyclopentanol (114) (prepared from components A.179 and C.36). ¹H NMR (DMSO-d₆) δ 8.63 (s, 1H), 8.47 (s, 1H), 8.21 (s, 1H), 7.55 (d, J=8.6 Hz, 1H), 7.34 (d, J=8.6 Hz, 1H), 5.25 (br, 1H), 3.57-3.52 (m, 1H), 3.31-3.26 (m, 1H), 3.21-3.15 (m, 2H), 2.55-2.45 (m, 1H), 2.28-2.20 (m, 1H)), 2.10-2.00 (m, 5H), 1.92-1.78 (m, 4H); ms 422.2 (M+H⁺).

### Example 115

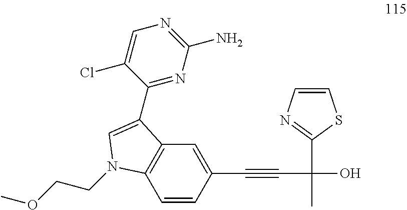

4-(3-(2-amino-5-chloropyrimidin-4-yl)-1-(2-methoxyethyl)-1H-indol-5-yl)-2-(thiazol-2-yl)but-3-yn-2-ol (115) (prepared from components A.171 and C.4). ¹H NMR (DMSO-d₆) δ 8.58 (s, 1H), 8.50 (s, 1H), 8.27 (s, 1H), 7.78 (d, J=3.1 Hz, 1H), 7.69 (d, J=3.1 Hz, 1H), 7.63 (d, J=8.8 Hz, 1H), 7.29 (d, J=8.8 Hz, 1H), 7.05-6.75 (br, 2H), 4.48 (t, J=5.3 Hz, 2 H), 3.70 (t, J=5.3 Hz, 2H), 3.24 (s, 3H), 1.93 (s, 3H); ms 454.1 (M+H⁺).

### Example 116

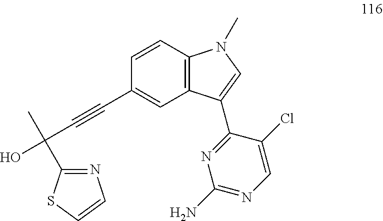

4-(3-(2-amino-5-chloropyrimidin-4-yl)-1-methyl-1H-indol-5-yl)-2-(thiazol-2-yl)but-3-yn-2-ol (116) (prepared from components A.xxx and C.4). ¹H NMR (methanol-d₄) δ 8.80 (s, 1 H), 8.40 (s, 1H), 8.16 (s, 1H), 7.79 (d, J=3.3 Hz, 1H), 7.58 (d, J=3.3 Hz, 1H), 7.42 (d, J=8.5 Hz, 1H), 7.37 (d, J=8.5 Hz, 1H), 5.51 (s, 1H), 3.90 (s, 3H), 2.01 (s, 3H); ms 410.0 (M+H⁺).

### Example 117

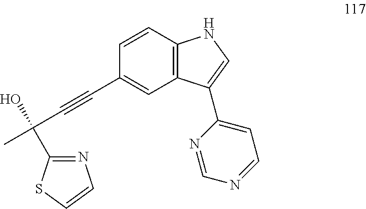

(2R)-4-(3-(pyrimidin-4-yl)-1H-indol-5-yl)-2-(thiazol-2-yl)but-3-yn-2-ol (117) (prepared from components A.184 and C.6). ¹H NMR (DMSO-d₆) δ 12.06 (s, 1H), 9.14 (s, 1H), 8.64 (s, 1H), 8.62 (s, 1H), 8.47 (d, J=2.4 Hz, 1H), 7.90 (d, J=5.6 Hz, 1H), 7.78 (d, J=2.4 Hz, 1 H), 7.68 (d, J=3.6 Hz, 1H), 7.48 (d, J=8.4 Hz, 1H), 7.24 (d, J=8.4 Hz, 1H), 6.92 (s, 1H), 1.90 (s, 3H); ms 347.1 (M+H⁺).

### Example 118

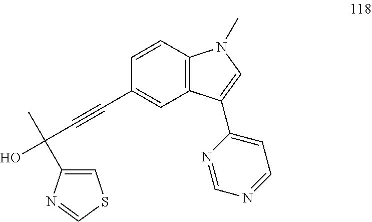

4-(1-methyl-3-(pyrimidin-4-yl)-1H-indol-5-yl)-2-(thiazol-4-yl)but-3-yn-2-ol (118) (prepared from components A.89 and C.29). ¹H NMR (DMSO-d₆) δ 9.13 (s, 1H), 9.05 (s, 1H), 8.64 (s, 1 H), 8.62 (s, 1H), 8.44 (s, 1H), 7.83 (d, J=4.2 Hz, 1H), 7.66 (s, 1H), 7.56 (d, J=8.8 Hz, 1 H), 7.32 (d, J=8.8 Hz, 1H), 6.29 (s, 1H), 3.90 (s, 3H), 1.85 (s, 3H); MS 361.1 (M+H⁺).

### Example 119

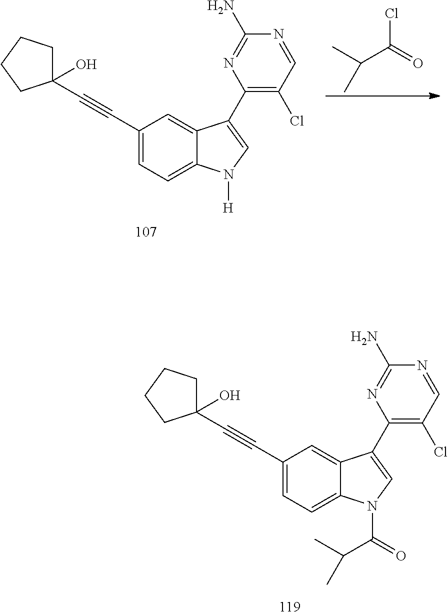

1-(3-(2-amino-5-chloropyrimidin-4-yl)-5-(2-(1-hydroxycyclopentyl)ethynyl)-1H-indol-1-yl)-2-methylpropan-1-one (119). To a mixture of 1-(2-(3-(2-amino-5-chloropyrimidin-4-yl)-1H-indol-5-yl)ethynyl)cyclopentanol (example 107) (15 mg, 0.042 mmol), triethylamine (6.5 μL, 0.046 mmol) and DMAP (1.1 mg, 0.008 mmol) in dichloromethane (2.0 mL) isobutyryl chloride (4.5 μL, 0.046 mmol) was added. The mixture was stirred for 1.5 h at room temperature, then concentrated. Purification of the residue by flash chromatography over silica gel, using 5:4.5:0.5 ethyl acetate-hexane-methanol, gave 1-(3-(2-amino-5-chloropyrimidin-4-yl)-5-(2-(1-hydroxycyclopentyl)ethynyl)-1H-indol-1-yl)-2-methylpropan-1-one (119) (5.6 mg, 31%): ¹H NMR (DMSO-d₆) δ 8.66 (s, 1H), 8.42 (d, J=8.5 Hz, 1H), 8.41 (s, 1H), 8.23 (s, 1H), 7.44 (d, J=8.5 Hz, 1H), 7.06 (s, 2H), 3.67-3.63 (m, 1H), 1.94-1.90 (m, 4H), 1.80-1.70 (m, 4 H), 1.29 (d, J=7.0 Hz, 6H); ms 423.1 (M+H⁺).

### Example 120-123

These examples were prepared by using the same or analogous synthetic techniques and substituting with appropriate reagents as example 119 starting from the compound of example 107.

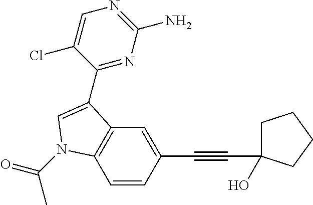

1-(3-(2-amino-5-chloropyrimidin-4-yl)-5-(2-(1-hydroxycyclopentyl)ethynyl)-1H-indol-1-yl)ethanone (120): ¹H NMR (DMSO-d₆) δ 8.57 (s, 1H), 8.41 (s, 1H), 8.37 (d, J=8.5 Hz, 1H), 8.26 (s, 1H), 7.44 (d, J=8.5 Hz, 1H), 7.05 (s, 2H), 2.76 (s, 3H), 1.96-1.92 (m, 4H), 1.80-1.70 (m, 4H); ms 395.4 (M+H⁺).

### Example 121

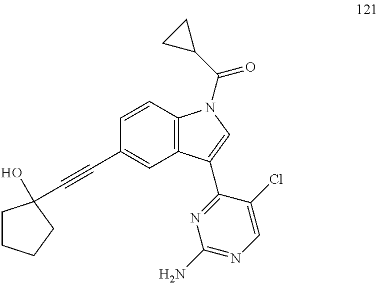

(3-(2-amino-5-chloropyrimidin-4-yl)-5-(2-(1-hydroxycyclopentyl)ethynyl)-1H-indol-1-yl)(cyclopropyl)methanone (121): ¹H NMR (DMSO-d₆) δ 8.89 (s, 1H), 8.42 (s, 1H), 8.63 (d, J=9.0 Hz, 1H), 8.24 (s, 1H), 7.43 (d, J=9.0 Hz, 1H), 7.06 (s, 2H), 2.81-2.75 (m, 1H), 1.95-1.90 (m, 4H), 1.80-1.65 (m, 4H), 1.20-1.15 (m, 4H); ms 421.4 (M+H⁺).

### Example 122

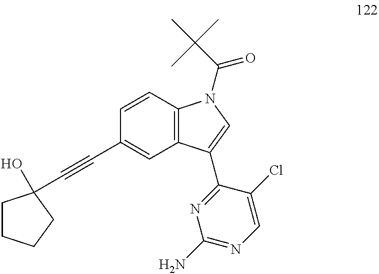

1-(3-(2-amino-5-chloropyrimidin-4-yl)-5-(2-(1-hydroxycyclopentyl)ethynyl)-1H-indol-1-yl)-2,2-dimethylpropan-1-one (122): ¹H NMR (DMSO-d₆) δ 8.77 (s, 1H), 8.41 (s, 1H), 8.39 (d, J=8.5 Hz, 1H), 8.29 (s, 1H), 7.44 (d, J=8.5 Hz, 1H), 7.07 (s, 2H), 1.96-1.85 (m, 4H), 1.80-1.70 (m, 4H), 1.52 (s, 9H); ms 437.4 (M+H⁺).

### Example 123

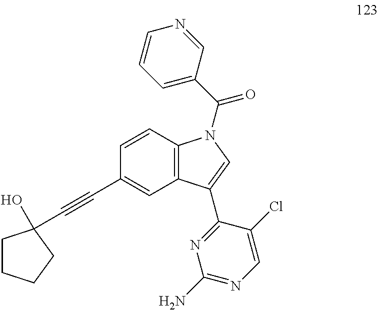

(3-(2-amino-5-chloropyrimidin-4-yl)-5-(2-(1-hydroxycyclopentyl)ethynyl)-1H-indol-1-yl)(pyridin-3-yl)methanone (123): ¹H NMR (DMSO-d₆) δ 9.03 (d, J=2.0 Hz, 1H), 8.89 (d, J=5.0 Hz, 1H), 8.39-8.34 (m, 3H), 8.28 (d, J=8.0 Hz, 1H), 8.22 (s, 1H), 7.68 (dd, J=7.5, 5.0 Hz, 1H), 7.52 (d, J=8.5 Hz, 1H), 7.07 (s, 2H), 1.96-1.85 (m, 4H), 1.81-1.70 (m, 4H); ms 458.0 (M+H⁺).

### Example 124

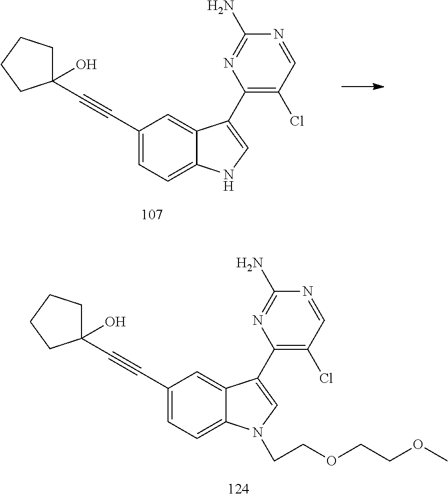

1-(2-(3-(2-amino-5-chloropyrimidin-4-yl)-1-(2-(2-methoxyethoxy)ethyl)-1H-indol-5-yl)ethynyl)cyclopentanol (124). A mixture of 1-(2-(3-(2-amino-5-chloropyrimidin-4-yl)-1H-indol-5-yl)ethynyl)cyclopentanol (107) (24.6 mg, 0.070 mmol), 1-bromo-2-(2-methoxyethoxy)ethane (0.04 mL, 0.21 mmol) and Cs₂CO₃ (45.3 mg, 0.139 mmol) in DMF (5 mL) was heated at 100° C. for 2.5 hrs. After cooled to room temperature, the mixture was diluted with ether, washed with water and brine, dried and concentrated. Purification of the residue by flash chromatography over silica gel, using 4.5:5:0.5 ethyl acetate-hexane-methanol, gave 1-(2-(3-(2-amino-5-chloropyrimidin-4-yl)-1-(2-(2-methoxyethoxy)ethyl)-1H-indol-5-yl)ethynyl)cyclopentanol (124) (20.7 mg, 65%): ¹H NMR (methanol-d₄) δ 8.69 (s, 1H), 8.50 (s, 1H), 8.20 (s, 1H), 7.49 (d, J=8.0 Hz, 1H), 7.32 (d, J=8.0 Hz, 1H), 4.44 (t, J=5.0 Hz, 2H), 3.86 (t, J=5.0 Hz, 2H), 3.56 (t, J=5.0 Hz, 2H), 3.48 (t, J=5.0 Hz, 2H), 3.28 (s, 3H), 2.18-2.03 (m, 4H), 1.94-1.78 (m, 4H); ms 455.2 (M+H⁺).

### Examples 125-139

These examples were prepared by using the same or analogous synthetic techniques and substituting with appropriate reagents as for example 124.

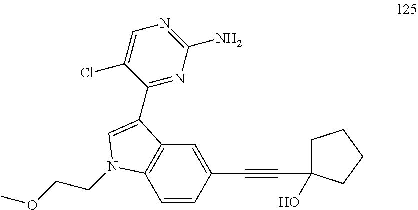

1-(2-(3-(2-amino-5-chloropyrimidin-4-yl)-1-(2-methoxyethyl)-1H-indol-5-yl)ethynyl)cyclopentanol (125): ¹H NMR (methanol-d₄) δ 8.69 (s, 1H), 8.47 (s, 1H), 8.21 (s, 1 H), 7.49 (d, J=8.5 Hz, 1H), 7.33 (d, J=8.5 Hz, 1H), 4.45 (t, J=5.0 Hz, 2H), 3.78 (t, J=5.0 Hz, 2H), 3.33 (s, 3H), 2.15-2.05 (m, 4H), 1.90-1.75 (m, 4H); ms 411.1 (M+H⁺).

### Example 126

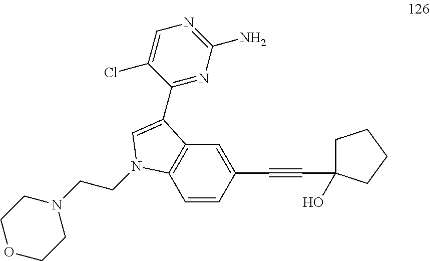

1-(2-(3-(2-amino-5-chloropyrimidin-4-yl)-1-(2-morpholinoethyl)-1H-indol-5-yl)ethynyl)cyclopentanol (126): ¹H NMR (DMSO-d₆) δ 8.59 (s, 1H), 8.58 (s, 1H), 8.26 (s, 1 H), 7.60 (d, J=8.5 Hz, 1H), δ 7.28 (d, J=8.5 Hz, 1H), 6.81 (s, 2H), 5.24 (s, 1H), 4.42 (t, J=5.0 Hz, 2H), 3.55 (br, 4H), 2.71 (t, J=5.0 Hz, 2H), 2.47 (br, 4H), 1.92-1.83 (m, 4H), 1.70-1.58 (m, 4H); ms 466.1 (M+H⁺).

### Example 127

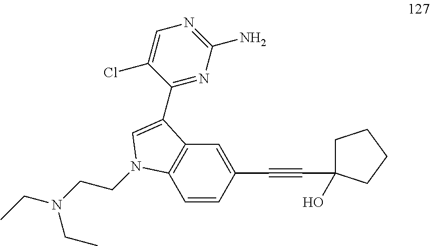

1-(2-(3-(2-amino-5-chloropyrimidin-4-yl)-1-(2-(diethylamino)ethyl)-1H-indol-5-yl)ethynyl)cyclopentanol (127): ¹H NMR (DMSO-d₆) δ 8.58 (s, 1H), 8.53 (s, 1H), 8.24 (s, 1 H), 7.57 (d, J=8.5 Hz, 1H), 7.26 (d, J=8.5 Hz, 1H), 5.24 (s, 1H), 4.33 (t, J=6.0 Hz, 2H), 2.76 (t, J=6.0 Hz, 2H), 2.48 (q, J=7.0 Hz, 4H), 1.92-1.83 (m, 4H), 1.70-1.58 (m, 4H), 0.84 (t, J=7.0 Hz, 6H); ms 452.2 (M+H⁺).

### Example 128

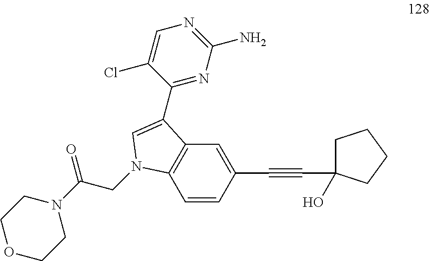

2-(3-(2-amino-5-chloropyrimidin-4-yl)-5-(2-(1-hydroxycyclopentyl)ethynyl)-1H-indol-1-yl)-1-morpholinoethanone (128): ¹H NMR (DMSO-d₆) δ 8.61 (s, 1H), 8.45 (s, 1H), 8.26 (s, 1 H), 7.45 (d, J=8.0 Hz, 1H), 7.26 (d, J=8.0 Hz, 1H), 6.83 (s, 2H), 5.37 (s, 1H), 3.72 (br, 2 H), 3.61 (br, 4H), 3.47 (br, 2H), 1.92-1.83 (m, 4H), 1.70-1.61 (m, 4H); ms 480.1 (M+H⁺).

### Example 129

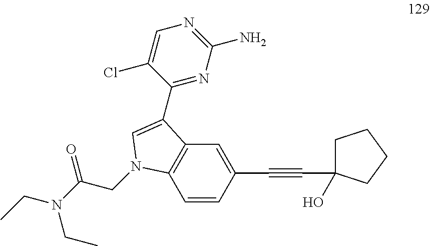

2-(3-(2-amino-5-chloropyrimidin-4-yl)-5-(2-(1-hydroxycyclopentyl)ethynyl)-1H-indol-1-yl)-N,N-diethylacetamide (129): ¹H NMR (DMSO-d₆) δ 8.60 (s, 1H), 8.47 (s, 1H), 8.23 (s, 1 H), 7.36 (d, J=8.4 Hz, 1H), 7.23 (d, J=8.4 Hz, 1H), 6.79 (s, 2H), 5.31 (s, 1H), 3.46 (q, J=6.8 Hz, 2H), 1.92-1.88 (m, 4H), 1.82-1.63 (m, 4H), 1.24 (t, J=6.8 Hz, 3H), 1.04 (t, J=6.8 Hz, 3H); ms 466.1 (M+H⁺).

### Example 130

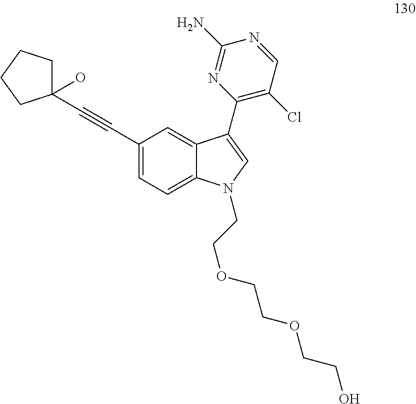

1-(2-(3-(2-amino-5-chloropyrimidin-4-yl)-1-(2-(2-hydroxyethoxy)ethoxy)ethyl)-1H-indol-5-yl)ethynyl)cyclopentanol (130): ¹H NMR (DMSO-d₆) δ 8.56 (s, 1H), 8.47 (s, 1H), 8.23 (s, 1H), 7.58 (d, J=8.2 Hz, 1H), 7.25 (d, J=8.2 Hz, 1H), 6.78 (s, 2H), 5.21 (s, 1H), 4.50 (t, J=5.6 Hz, 1H), 4.45 (t, J=4.9 Hz, 2H), 3.77 (t, J=4.9 Hz, 2H), 3.54-3.47 (m, 2H), 3.46-3.42 (m, 2H), 3.41 (t, J=4.9 Hz, 2H), 3.36 (d, J=5.6 Hz, 2H), 1.92-1.73 (m, 4H), 1.70-1.61 (m, 4H); ms 485.2 (M+H⁺).

### Example 131

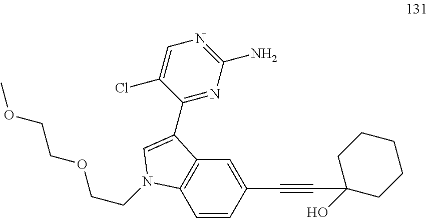

1-(2-(3-(2-amino-5-chloropyrimidin-4-yl)-1-(2-(2-methoxyethoxy)ethyl)-1H-indol-5-yl)ethynyl)cyclohexanol (131) starting with the compound of example 109. ¹H NMR (DMSO-d₆) δ 8.56 (s, 1H), 8.51 (s, 1H), 8.27 (s, 1H), 7.61 (d, J=8.0 Hz, 1H), 7.28 (d, J=8.0 Hz, 1H), 6.89 (br, 2H), 4.48 (br, 2H), 3.79 (br, 2H), 3.19 (s, 3H), 1.92-1.80 (m, 2H), 1.70-1.48 (m, 7H), 1.30-1.15 (m, 1H); ms 469.1 (M+H⁺).

### Example 132

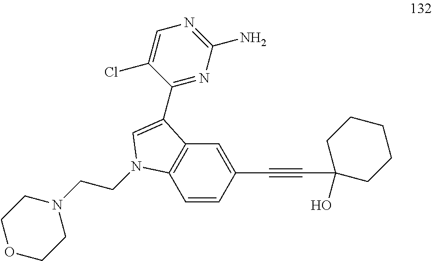

1-(2-(3-(2-amino-5-chloropyrimidin-4-yl)-1-(2-morpholinoethyl)-1H-indol-5-yl)ethynyl)cyclohexanol (132) starting with the compound of example 109. ¹H NMR (DMSO-d₆) δ 8.55 (s, 1H), 8.54 (s, 1H), 8.24 (s, 1H), 7.58 (d, J=8.5 Hz, 1H), 7.27 (d, J=8.5 Hz, 1H), 6.79 (br, 2H), 5.29 (br, 1H), 4.39 (t, J=5.0 Hz, 2H), 3.54 (br, 4H), 2.71 (t, J=5.0 Hz, 2H), 2.49 (br, 4H), 1.90-1.78 (m, 2H), 1.68-1.49 (m, 7H), 1.35-1.15 (m, 1H); ms 480.2 (M+H⁺).

### Example 133

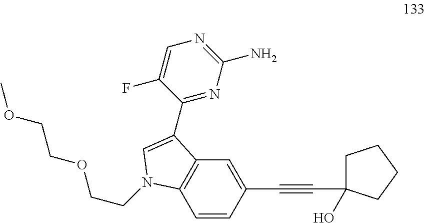

1-(2-(3-(2-amino-5-fluoropyrimidin-4-yl)-1-(2-(2-methoxyethoxy)ethyl)-1H-indol-5-yl)ethynyl)cyclopentanol (133) starting with the compound of example 95. ¹H NMR (DMSO-d₆) 8.72 (d, J=1.5 Hz, 1H), 8.27 (m, 2H), 7.64 (d, J=8.5 Hz, 1H), 7.30 (dd, J=8.5, 1.5 Hz, 1H), 6.75 (br, 2H), 4.48 (t, J=5.0 Hz, 2H), 3.80 (t, J=5.0 Hz, 2H), 3.53 (t, J=5.0 Hz, 2H), 3.38 (t, J=5.0 Hz, 2H), 3.18 (s, 3H), 1.90-1.82 (m, 4H), 1.70-1.62 (m, 4H); ms 439.1 (M+H⁺).

### Example 134

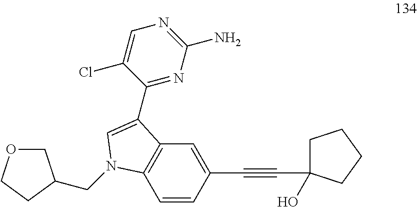

1-(2-(3-(2-amino-5-chloropyrimidin-4-yl)-1-((tetrahydrofuran-3-yl)methyl)-1H-indol-5-yl)ethynyl)cyclopentanol (134): ¹H NMR (DMSO-d₆) δ 8.55 (s, 1H), 8.51 (s, 1H), 8.26 (s, 1 H), 7.64 (d, J=8.5 Hz, 1H), 7.27 (d, J=8.5 Hz, 1H), 6.82 (br, 1H), 4.35-4.26 (m, 2H), 3.88-3.83 (m, 1H), 3.70-3.61 (m, 2H), 3.51-3.45 (m, 1H), 2.85-2.76 (m, 1H), 1.97-1.85 (m, 5 H), 1.81-1.63 (m, 5H); ms 437.2 (M+H⁺).

### Example 135

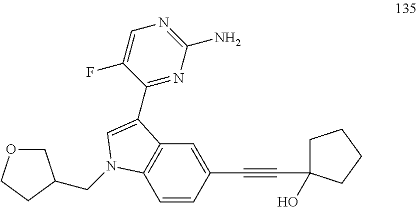

1-(2-(3-(2-amino-5-fluoropyrimidin-4-yl)-1-((tetrahydrofuran-3-yl)methyl)-1H-indol-5-yl)ethynyl)cyclopentanol (135) starting with the compound of example 95. ¹H NMR (DMSO-d₆) 8.72 (s, 1H), 8.23 (m, 2H), 7.65 (d, J=8.5 Hz, 1H), 7.30 (dd, J=8.5, 1.5 Hz, 1 H), 6.56 (br, 2H), 5.26 (s, 1H), 4.35-4.26 (m, 2H), 3.88-3.83 (m, 1H), 3.70-3.61 (m, 2H), 3.51-3.45 (m, 1H), 2.85-2.76 (m, 1H), 1.99-1.85 (m, 5H), 1.83-1.63 (m, 5H); ms 421.1 (M+H⁺).

### Example 136

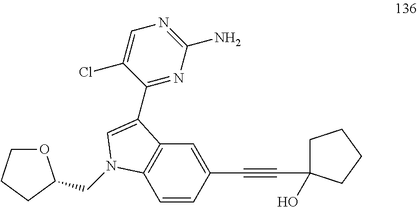

1-(2-(3-(2-amino-5-chloropyrimidin-4-yl)-1-(((S)-tetrahydrofuran-2-yl)methyl)-1H-indol-5-yl)ethynyl)cyclopentanol (136): ¹H NMR (DMSO-d₆) δ 8.56 (s, 1H), 8.47 (s, 1H), 8.26 (s, 1 H), 7.62 (d, J=8.5 Hz, 1H), 7.26 (d, J=8.5 Hz, 1H), 6.82 (br, 2H), 5.25 (s, 1H), 4.45-4.40 (m, 1H), 4.35-4.28 (m, 1H), 4.22-4.15 (m, 1H), 3.78-3.72 (m, 1H), 3.67-3.60 (m, 1H), 3.39-3.26 (m, 2H), 2.05-1.90 (m, 4H), 1.78-1.68 (m, 5H), 1.59-1.53 (m, 1H); ms 437.2 (M+H⁺).

### Example 137

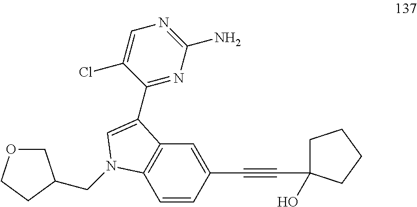

1-(2-(3-(2-amino-5-chloropyrimidin-4-yl)-1-((-tetrahydrofuran-3-yl)methyl)-1H-indol-5-yl)ethynyl)cyclopentanol (137): ¹H NMR (DMSO-d₆) δ 8.53 (s, 1H), 8.48 (s, 1H), 8.24 (s, 1 H), 7.62 (d, J=8.5 Hz, 1H), 7.26 (d, J=8.5 Hz, 1H), 6.80 (br, 2H), 5.22 (s, 1H), 4.45-4.20 (m, 1H), 3.85-3.75 (m, 2H), 3.50-3.40 (m, 1H), 3.35-3.25 (m, 2H), 2.85-2.70 (m, 1H), 1.95-1.80 (m, 5H), 1.78-1.55 (m, 5H); ms 437.2 (M+H⁺).

### Example 138

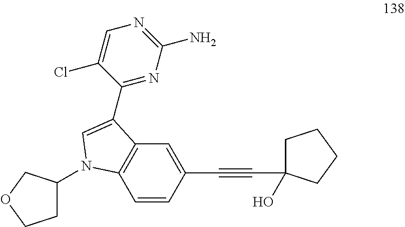

1-(2-(3-(2-amino-5-chloropyrimidin-4-yl)-1-(tetrahydrofuran-3-yl)-1H-indol-5-yl)ethynyl)cyclopentanol (138): ¹H NMR (DMSO-d₆) δ 8.55 (s, 1H), 8.42 (s, 1H), 8.27 (s, 1 H), 7.68 (d, J=8.5 Hz, 1H), 7.30 (d, J=8.5 Hz, 1H), 6.84 (br, 2H), 5.39 (s, 1H), 4.12-3.97 (m, 4H), 3.90-3.84 (m, 1H), 2.64-2.52 (m, 1H), 2.21-2.15 (m, 1H), 1.97-1.93 (m, 4H), 1.79-1.68 (m, 4H); ms 423.1 (M+H⁺).

### Example 139

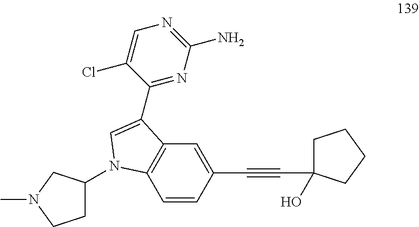

1-(2-(3-(2-amino-5-chloropyrimidin-4-yl)-1-(1-methylpyrrolidin-3-yl)-1H-indol-5-yl)ethynyl)cyclopentanol (139): ¹H NMR (DMSO-d₆) δ 8.56 (s, 1H), 8.51 (s, 1H), 8.24 (s, 1 H), 7.72 (d, J=8.6 Hz, 1H), 7.25 (d, J=8.6 Hz, 1H), 6.81 (br, 2H), 5.25 (br, 1H), 3.08-3.00 (m, 1H), 2.98-2.93 (m, 1H), 2.72-2.65 (m, 1H), 2.55-2.45 (m, 2H), 2.35 (s, 3H), 2.35-2.25 (m, 1H)), 1.98-1.90 (m, 5H), 1.80-1.65 (m, 4H); ms 436.1 (M+H⁺).

### Example 140

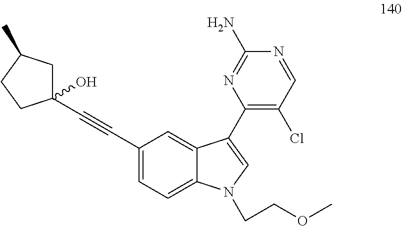

### (3R)-1-(2-(3-(2-amino-5-chloropyrimidin-4-yl)-1-(2-methoxyethyl)-1H-indol-5-yl)ethynyl)-3-methylcyclopentanol

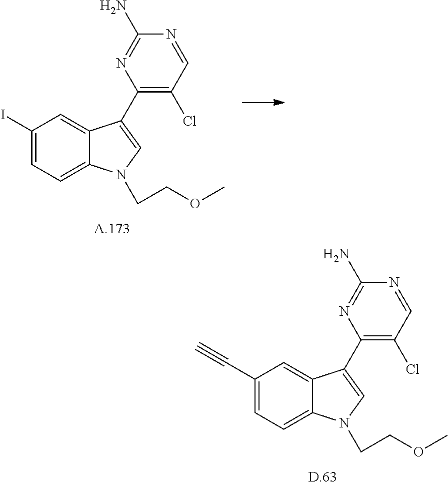

5-chloro-4-(5-ethynyl-1-(2-methoxyethyl)-1H-indol-3-yl)pyrimidin-2-amine D.63: A mixture of 5-chloro-4-(5-iodo-1-(2-methoxyethyl)-1H-indol-3-yl)pyrimidin-2-amine A.173 (340 mg, 0.794 mmol), ethynyltrimethylsilane (0.2 mL), PdCl₂(PPh₃)₂ (55.7 mg, 0.0794 mmol), and copper(I) iodide (30.2 mg, 0.159 mmol) in triethylamine (4.0 mL), DMF (4.0 mL) was stirred at room temperature under N₂ for 10 min. The resultant mixture was diluted with ether, washed with water and brine, dried and concentrated. Purification of the residue by flash chromatography over silica gel, using 5:4:0.5 ethyl acetate-hexane-methanol, gave a crude compound (320 mg); ms 399.1 (M+H⁺). The crude compound was dissolved in THF-methanol (1:2, 15 mL) and treated with K₂CO₃ (74.2 mg, 0.537 mmol). The resultant mixture was stirred at room temperature for 2 hrs and diluted with dichloromethane, filtered through a pad of Celite and concentrated to afford pure 5-chloro-4-(5-ethynyl-1-(2-methoxyethyl)-1H-indol-3-yl)pyrimidin-2-amine D.63 (182.9 mg, 71%): ¹H NMR (DMSO-d₆) δ 8.78 (s, 1H), 8.56 (s, 1H), 8.23 (s, 1H), 7.61 (d, J=8.0 Hz, 1H), 7.34 (d, J=8.0 Hz, 1H), 6.82 (s, 2H), 4.49 (t, J=4.8 Hz, 2H), 4.07 (s, 1H), 3.70 (t, J=4.8 Hz, 2H), 3.23 (s, 3H).

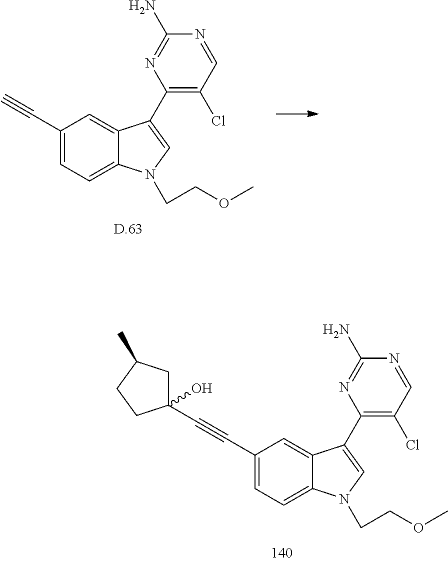

(3R)-1-(2-(3-(2-amino-5-chloropyrimidin-4-yl)-1-(2-methoxyethyl)-1H-indol-5-yl)ethynyl)-3-methylcyclopentanol (140): To 5-chloro-4-(5-ethynyl-1-(2-methoxyethyl)-1H-indol-3-yl)pyrimidin-2-amine D.63 (50.0 mg, 0.153 mmol) in THF (5.0 mL) was added BuLi (2.5M in Hex, 0.12 mL, 0.31 mmol) at 0° C., and the resultant solution was stirred for 10 min at 0° C. To the mixture (R)-3-methylcyclopentanone (0.033 mL, 0.31 mmol) was added and the stirring was continued for 2 hrs at 0° C. The resultant mixture was diluted with ether, washed with water and brine, dried and concentrated. Purification of the residue by flash chromatography over silica gel, using 5:4.5:0.2 ethyl acetate-hexane-methanol, gave (3R)-1-(2-(3-(2-amino-5-chloropyrimidin-4-yl)-1-(2-methoxyethyl)-1H-indol-5-yl)ethynyl)-3-methylcyclopentanol (140)(12.8 mg, 20%): ¹H NMR (DMSO-d₆) δ 8.56 (s, 1H), 8.47 (s, 1H), 8.25 (s, 1H), 7.60 (d, J=8.2 Hz, 1H), 7.25 (d, J=8.2 Hz, 1H), 6.82 (s, 2H), 4.47 (br, 2H), 3.71 (br, 2H), 3.24 (s, 3H), 2.31-2.22 (m, 1H), 2.22-2.10 (m, 1H), 2.00-1.89 (m, 2H), 1.89-1.82 (m, 1H), 1.58-1.48 (m, 1H), 1.41-1.33 (m, 1H), 1.32-1.20 (m, 1H), 1.10-1.05 (m, 3H); ESI-MS: 425.2 (M+H⁺).

### Example 143

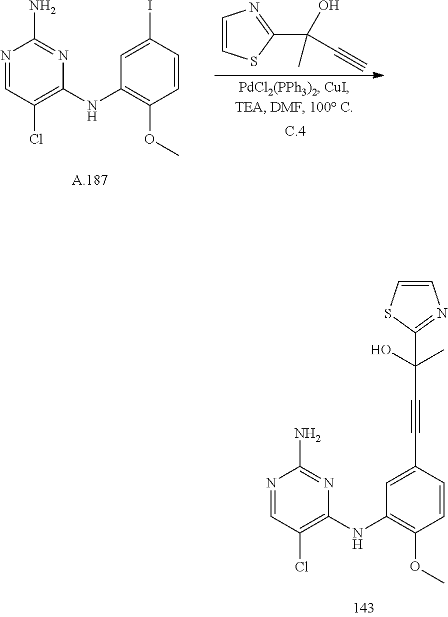

4-(3-((2-amino-5-chloro-4-pyrimidinyl)amino)-4-(methyloxy)phenyl)-2-(1,3-thiazol-2-yl)-3-butyn-2-ol (143). To a flask containing 5-chloro-N⁴-(5-iodo-2-methoxyphenyl)-pyrimidine-2,4-diamine (A.187)(128.6 mg, 0.34 mmol), 2-(thiazol-2-yl)but-3-yn-2-ol C.4 (157 mg, 1.0 mmol), copper (I) iodide (13.0 mg, 0.068 mmol), and PdCl₂(PPh₃)₂ (24 mg, 0.034 mmol) was added dry DMF (3.00 mL) and triethylamine (3.00 mL). The mixture was heated to 100° C. After 3.5 hours, the mixture was cooled to room temperature then diluted with water. After extracting three times with ethyl acetate, the organic solvent was removed under reduced pressure. The residue was purified by silica gel flash chromatography (0-100% methanol in dichloromethane) to afford 4-(3-((2-amino-5-chloro-4-pyrimidinyl)amino)-4-(methyloxy)phenyl)-2-(1,3-thiazol-2-yl)-3-Butyn-2-ol (143) (50 mg, 36.4%). ¹H NMR (500 MHz, DMSO-d₆) δ ppm 8.16 (1H, m), 7.96 (1H, m), 7.87 (1H, m), 7.76 (1H, m), 7.67 (1H, m), 7.16 (1H, d, J=7.8 Hz), 7.08 (1H, d, J=7.3 Hz), 6.93 (1H, m), 6.49 (2H, m), 3.89 (3H, s), 1.88 (3H, s). MS ESI (pos.) m/e: 402.1 (M+H).

Examples 144-152 were prepared using same or analogous synthetic techniques and substituting with appropriate reagent as in example 143.

### Example 144

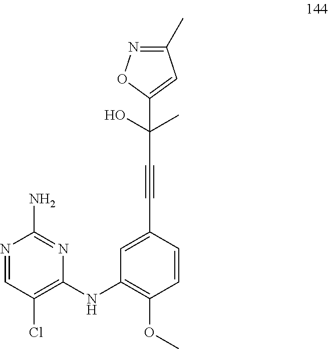

4-(3-((2-amino-5-chloro-4-pyrimidinyl)amino)-4-(methyloxy)phenyl)-2-(3-methyl-5-isoxazolyl)-3-butyn-2-ol (144) (prepared from components A.187 and C.24). ¹H NMR (500 MHz, DMSO-d₆) δ ppm 8.23 (1H, d, J=2.0 Hz), 7.97 (1H, s), 7.87 (1H, s), 7.20 (1H, dd, J=8.4, 2.1 Hz), 7.09 (1H, d, J=8.6 Hz), 6.62 (1H, s), 6.52 (2H, s), 6.38 (1H, s), 3.90 (3H, s), 2.23 (3H, s), 1.80 (3H, s). MS ESI (pos.) m/e: 400.1 (M+H).

### Example 145

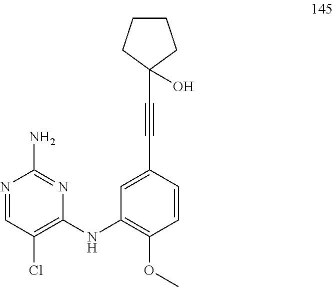

1-((3-((2-amino-5-chloro-4-pyrimidinyl)amino)-4-(methyloxy)-phenyl)ethynyl)-cyclopentanol (145) (prepared from components A.187 and C.36). ¹H NMR (500 MHz, DMSO-d₆) δ ppm 8.18 (1H, m), 7.97 (1H, s), 7.85 (1H, s), 7.12 (1H, m), 7.08 (1H, m), 6.53 (2H, s), 5.22 (1H, s), 3.89 (3H, s), 1.90 (5H, m), 1.69 (4H, m). MS ESI (pos.) m/e: 359.0 (M+H).

### Example 146

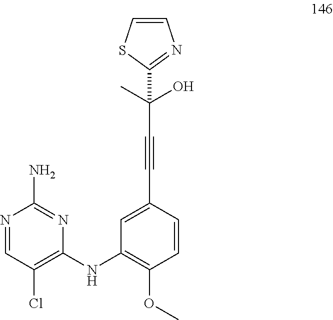

(R)-4-(3-(2-Amino-5-chloropyrimidin-4-ylamino)-4-methoxyphenyl)-2-(thiazol-2-yl)but-3-yn-2-ol (146) (prepared from components A.187 and C.6). ¹H NMR (500 MHz, DMSO-d₆) δ ppm 8.16 (1H, d, J=2.0 Hz), 7.96 (1H, s), 7.87 (1H, s), 7.76 (1H, d, J=3.4 Hz), 7.67 (1H, d, J=3.2 Hz), 7.14-7.17 (1H, m), 7.08 (1H, d, J=8.6 Hz), 6.93 (1H, s), 6.49 (2H, s), 3.89 (3H, s), 1.88 (3H, s). MS ESI (pos.) m/e: 402.1 (M+H).

### Example 147

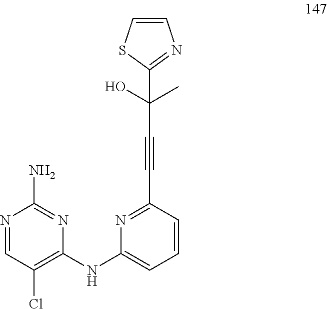

4-(6-(2-Amino-5-chloropyrimidin-4-ylamino)pyridin-2-yl)-2-(thiazol-2-yl)but-3-yn-2-ol (147) (prepared from components A.189 and E, 4). ¹H NMR (500 MHz, DMSO-d₆) δ ppm 8.25 (1H, s), 8.19 (1H, d, J=8.3 Hz), 7.87 (1H, t, J=7.8 Hz), 7.78 (1H, d, J=2.4 Hz), 7.70 (1H, d, J=2.7 Hz), 7.34 (1H, d, J=7.1 Hz), 1.88 (3H, s). MS ESI (pos.) m/e: 373.0 (M+H).

### Example 148

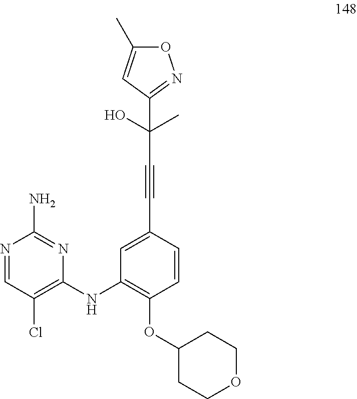

4-(3-(2-Amino-5-chloropyrimidin-4-ylamino)-4-(tetrahydro-2H-pyran-4-yloxy)phenyl)-2-(5-methylisoxazol-3-yl)but-3-yn-2-ol (148) (prepared from components A.193 and C.1). ¹H NMR (500 MHz, DMSO-d₆) δ ppm 8.34 (1H, d, J=2.0 Hz), 8.00 (2H, m), 7.17 (1H, m), 7.12 (1H, m), 6.57 (2H, s), 6.41 (2H, m), 4.75 (1H, tt, J=7.7, 3.8 Hz), 3.83 (2H, m), 3.52 (2H, ddd, J=11.4, 8.1, 3.2 Hz), 2.43 (3H, s), 2.02 (2H, m), 1.80 (3H, s), 1.67 (2H, m). MS ESI (pos.) m/e: 470.2 (M+H).

### Example 149

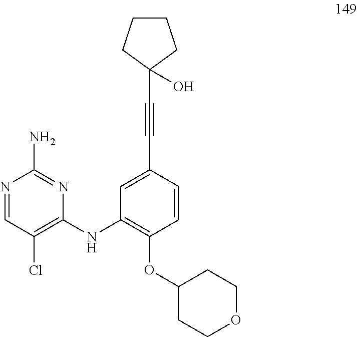

1-(2-(3-(2-Amino-5-chloropyrimidin-4-ylamino)-4-(tetrahydro-2H-pyran-4-yloxy)phenyl)ethynyl)cyclopentanol (149) (prepared from components A.193 and C.36). ¹H NMR (500 MHz, DMSO-d₆) δ ppm 8.33 (1H, d, J=2.0 Hz), 8.00 (2H, m), 7.16 (1H, m), 7.07 (1H, dd, J=8.4, 2.1 Hz), 6.59 (2H, s), 5.23 (1H, m), 4.73 (1H, dq, J=7.7, 4.0 Hz), 3.84 (2H, m), 3.52 (2H, ddd, J=11.6, 8.2, 3.2 Hz), 2.02 (2H, m), 1.94 (4H, m), 1.76 (2H, m), 1.69 (3 H, m). MS ESI (pos.) m/e: 429.1 (M+H).

### Example 150

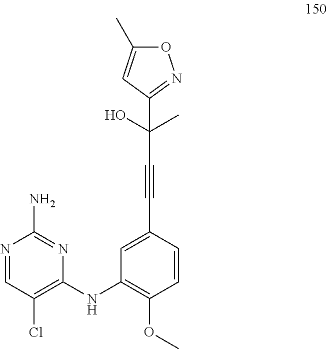

4-(3-(2-Amino-5-chloropyrimidin-4-ylamino)-4-methoxyphenyl)-2-(5-methylisoxazol-3-yl)but-3-yn-2-ol (150) (prepared from components A.187 and C.1). ¹H NMR (500 MHz, DMSO-d₆) δ ppm 8.19 (1H, t, J=2.0 Hz), 7.96 (1H, d, J=2.2 Hz), 7.87 (1H, d, J=1.5 Hz), 7.16 (1H, dt, J=8.5, 2.0 Hz), 7.08 (1H, dd, J=8.6, 2.0 Hz), 6.51 (2H, s), 6.40 (1H, d, J=2.0 Hz), 6.36 (1H, s), 3.91 (3H, m), 2.40 (3H, d, J=1.2 Hz), 1.80 (3H, d, J=1.7 Hz). MS ESI (pos.) m/e: 400.1 (M+H).

### Example 151

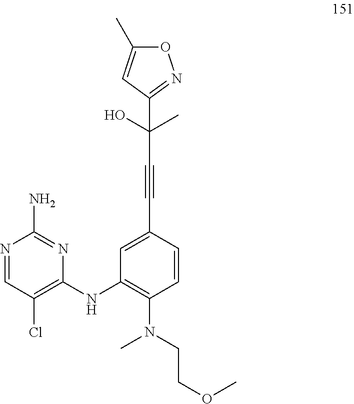

4-(3-(2-Amino-5-chloropyrimidin-4-ylamino)-4-((2-methoxyethyl)(methyl)amino)phenyl)-2-(5-methylisoxazol-3-yl)but-3-yn-2-ol (151) (prepared from components A.196 and C.1). ¹H NMR (500 MHz, DMSO-d₆) δ ppm 8.61 (1H, s), 8.32 (1H, d, J=1.7 Hz), 7.98 (1H, s), 7.26 (1 H, d, J=8.3 Hz), 7.09 (1H, dd, J=8.2, 1.6 Hz), 6.55 (2H, s), 6.42 (1H, s), 6.36 (1H, s), 3.38 (2 H, t, J=5.5 Hz), 3.19 (3H, m), 3.01 (2H, t, J=5.5 Hz), 2.67 (3H, s), 2.42 (3H, m), 1.80 (3H, s). MS ESI (pos.) m/e: 457.1 (M+H).

### Example 152

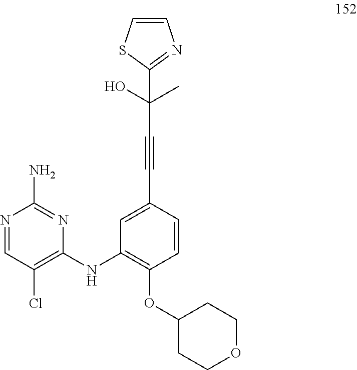

4-(3-(2-Amino-5-chloropyrimidin-4-ylamino)-4-(tetrahydro-2H-pyran-4-yloxy)phenyl)-2-(thiazol-2-yl)but-3-yn-2-ol (152) (prepared from components A.193 and E, 4). ¹H NMR (500 MHz, CHLOROFORM-d) δ ppm 8.65 (1H, d, J=2.0 Hz), 8.09 (1H, s), 7.94 (1H, s), 7.79 (1 H, d, J=3.4 Hz), 7.36 (1H, d, J=3.2 Hz), 7.25 (8H, s), 7.18 (1H, dd, J=8.4, 2.1 Hz), 6.87 (1H, d, J=8.6 Hz), 5.47 (1H, m), 4.67 (1H, m), 4.02 (2H, m), 3.64 (1H, ddd, J=11.6, 7.9, 3.4 Hz), 2.12 (4H, m), 1.90 (1H, m). MS ESI (pos.) m/e: 472.1 (M+H).

### Example 153 and 154

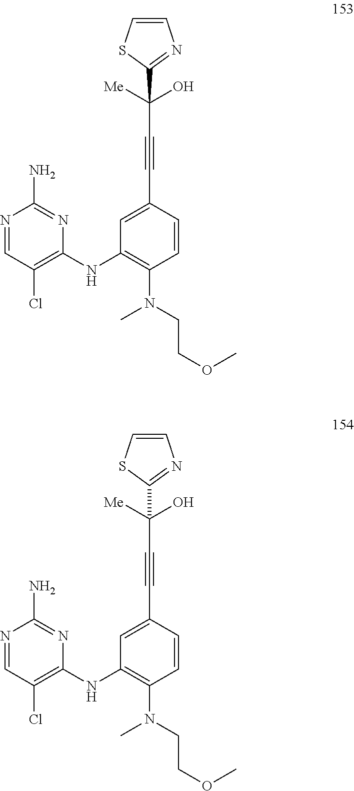

(S)-4-(3-(2-Amino-5-chloropyrimidin-4-ylamino)-4-((2-methoxyethyl)(methyl)-amino)phenyl)-2-(thiazol-2-yl)but-3-yn-2-ol (153) and (R)-4-(3-(2-Amino-5-chloropyrimidin-4-ylamino)-4-((2-methoxyethyl)(methyl)amino)phenyl)-2-(thiazol-2-yl)but-3-yn-2-ol (154). The racemic mixture of example 152 was resolved by chiral HPLC using an AD-H column with isocratic 20% isopropanol in hexanes. The first compound to elute off the column was assigned as (S)-4-(3-(2-Amino-5-chloropyrimidin-4-ylamino)-4-02-methoxyethyl)(methyl)amino)phenyl)-2-(thiazol-2-yl)but-3-yn-2-ol (153). The second compound to elute off the column was assigned as (R)-4-(3-(2-Amino-5-chloropyrimidin-4-ylamino)-4-((2-methoxyethyl)(methyl)amino)phenyl)-2-(thiazol-2-yl)but-3-yn-2-ol (154), based on bioactivity and analogy to other active isomers where the stereocenter was confirmed by crystallography.

(R)-4-(3-(2-Amino-5-chloropyrimidin-4-ylamino)-4-((2-methoxyethyl)(methyl)amino)phenyl)-2-(thiazol-2-yl)but-3-yn-2-ol (154) (8.0 mg). ¹H NMR (500 MHz, CHLOROFORM-d) δ ppm 8.77 (1H, s), 8.49 (1H, s), 7.83 (1H, s), 7.34 (1 H, s), 7.23 (1H, m), 7.17 (1H, m), 5.48 (2H, s), 3.41 (2H, t, J=5.5 Hz), 3.30 (3H, m), 3.09 (2 H, m), 2.71 (3H, s), 2.09 (3H, s). MS ESI (pos.) m/e: 459.1 (M+H).

### Example 155

Examples 155-161 were prepared using same or analogous synthetic techniques and substituting with appropriate reagent as in example 143.

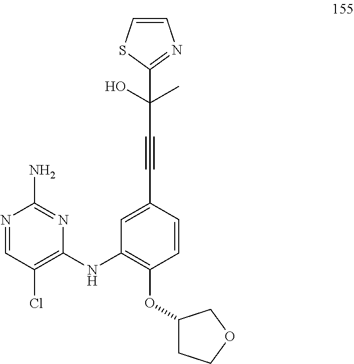

4-(3-(2-Amino-5-chloropyrimidin-4-ylamino)-4-(S)-tetrahydrofuran-3-yloxy)phenyl)-2-(thiazol-2-yl)but-3-yn-2-ol (155) (prepared from components A.199 and C.4). ¹H NMR (500 MHz, DMSO-d₆) δ ppm 8.28 (1H, d, J=1.7 Hz), 7.99 (1H, s), 7.92 (1H, s), 7.77 (1H, d, J=3.2 Hz), 7.68 (1H, d, J=3.2 Hz), 7.15 (2H, m), 6.94 (1H, s), 6.54 (2H, s), 5.17 (1H, s), 3.91 (1H, dd, J=10.4, 4.3 Hz), 3.86 (3H, m), 2.24 (1H, td, J=13.8, 8.1 Hz), 2.05 (1H, m), 1.90 (3H, s). MS ESI (pos.) m/e: 458.0 (M+H).

### Example 156

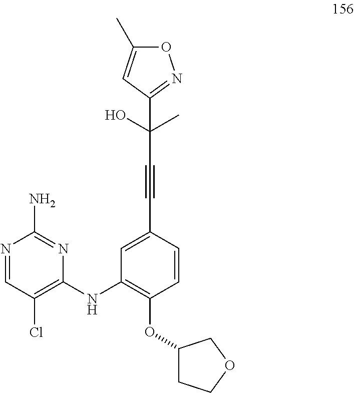

4-(3-(2-Amino-5-chloropyrimidin-4-ylamino)-4-(S)-tetrahydrofuran-3-yloxy)phenyl)-2-(5-methylisoxazol-3-yl)but-3-yn-2-ol (156) (prepared from components A.199 and C.1). ¹H NMR (500 MHz, DMSO-d₆) δ ppm 8.29 (1H, d, J=2.0 Hz), 7.98 (1H, m), 7.91 (1H, s), 7.14 (2H, m), 6.55 (2H, s), 6.41 (2H, m), 5.18 (1H, m), 3.91 (1H, dd, J=10.3, 4.4 Hz), 3.84 (3H, m), 2.40 (3H, s), 2.27 (1H, m), 2.03 (1H, m), 1.80 (3H, s). MS ESI (pos.) m/e: 456.1 (M+H).

### Example 157

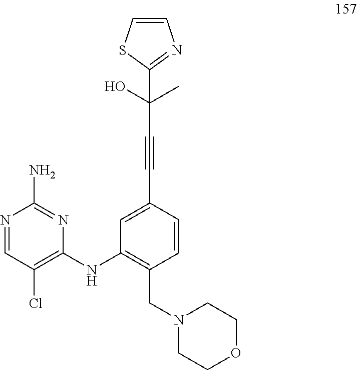

4-(3-(2-Amino-5-chloropyrimidin-4-ylamino)-4-(morpholinomethyl)phenyl)-2-(thiazol-2-yl)but-3-yn-2-ol (157) (prepared from components A.204 and C.4). ¹H NMR (500 MHz, DMSO-d₆) δ ppm 9.89 (1H, s), 8.22 (1H, d, J=1.2 Hz), 8.00 (1H, s), 7.78 (1H, d, J=3.2 Hz), 7.69 (1H, d, J=2.9 Hz), 7.25 (1H, d, J=7.8 Hz), 7.06 (1H, dd, J=7.6, 1.2 Hz), 6.99 (1H, s), 6.48 (2H, s), 3.60 (6H, m,), 2.39 (4H, m), 1.90 (3H, s). MS ESI (pos.) m/e: 471.1 (M+H).

### Example 158

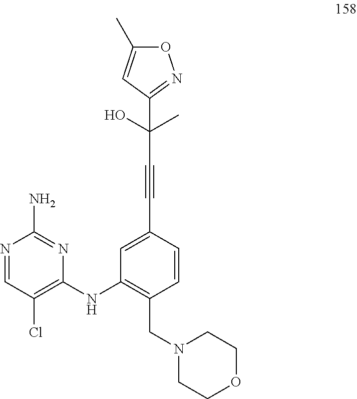

4-(3-(2-Amino-5-chloropyrimidin-4-ylamino)-4-(morpholinomethyl)phenyl)-2-(5-methylisoxazol-3-yl)but-3-yn-2-ol (158) (prepared from components A.204 and C.1). ¹H NMR (500 MHz, DMSO-d₆) δ ppm 9.89 (1H, s), 8.23 (1H, s), 7.99 (1H, s), 7.24 (1H, d, J=7.6 Hz), 7.06 (1H, d, J=7.8 Hz), 6.47 (3H, d, J=17.9 Hz), 6.36 (1H, s), 3.60 (6H, m), 2.40 (7H, m), 1.80 (3H, s). MS ESI (pos.) m/e: 469.1 (M+H).

### Example 159

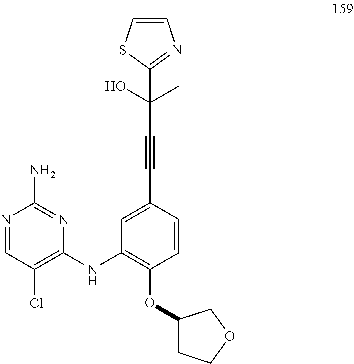

4-(3-(2-Amino-5-chloropyrimidin-4-ylamino)-4-(R)-tetrahydrofuran-3-yloxy)phenyl)-2-(thiazol-2-yl)but-3-yn-2-ol (159) (prepared from components A.207 and C.4). ¹H NMR (500 MHz, DMSO-d₆) δ ppm 8.28 (1H, s), 7.99 (1H, s), 7.92 (1H, s), 7.77 (1H, d, J=2.9 Hz), 7.68 (1H, d, J=3.2 Hz), 7.14 (2H, m), 6.94 (1H, s), 6.54 (2H, s), 5.17 (1H, s), 3.94 (1H, m), 3.79 (3H, t, J=9.0 Hz), 2.24 (1H, m), 2.00 (1H, m), 1.90 (3H, s). MS ESI (pos.) m/e: 458.0 (M+H).

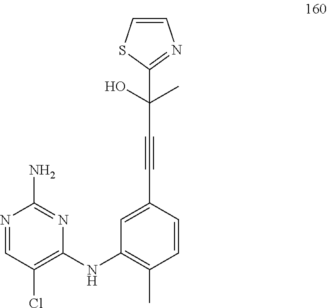

4-(3-(2-Amino-5-chloropyrimidin-4-ylamino)-4-methylphenyl)-2-(thiazol-2-yl)but-3-yn-2-ol (160) (prepared from components A.209 and C.4). ¹H NMR (500 MHz, DMSO-d₆) δ ppm 8.31 (1H, s), 7.86 (1H, s), 7.76 (1H, d, J=3.4 Hz), 7.67 (1H, d, J=3.2 Hz), 7.31 (2H, m), 7.21 (1H, m), 6.98 (1H, s), 6.22 (2H, s), 2.16 (3H, s), 1.86 (3H, s). MS ESI (pos.) m/e: 385.9 (M+H).

### Example 161

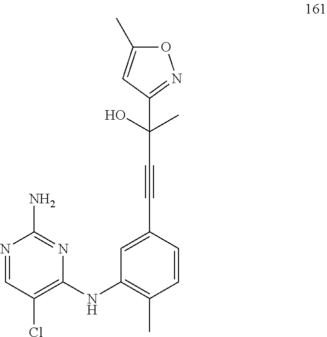

4-(3-(2-Amino-5-chloropyrimidin-4-ylamino)-4-methylphenyl)-2-(5-methylisoxazol-3-yl)but-3-yn-2-ol (161) (prepared from components A.209 and C.1). ¹H NMR (500 MHz, DMSO-d₆) δ ppm 8.30 (1H, s), 7.87 (1H, s), 7.31 (1H, s), 7.28 (1H, m), 7.22 (1H, m), 6.45 (1H, s), 6.34 (1H, s), 6.22 (2H, s), 2.35-2.41 (3H, m), 2.17 (3H, s), 1.77 (3H, s). MS ESI (pos.) m/e: 384.0 (M+H).

### Example 162 and 163

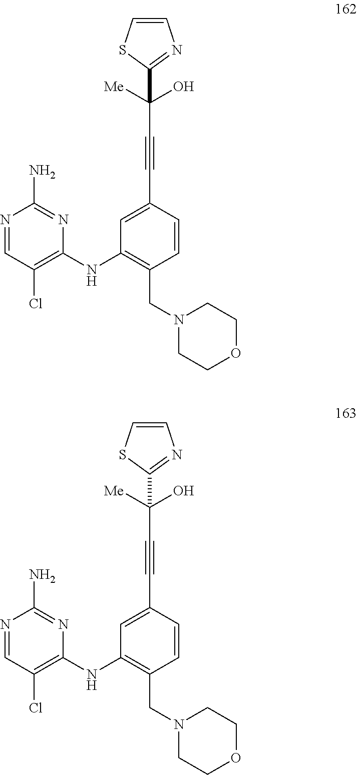

(S)-4-(3-(2-Amino-5-chloropyrimidin-4-ylamino)-4-(morpholinomethyl)phenyl)-2-(thiazol-2-yl)but-3-yn-2-ol (162) and (R)-4-(3-(2-Amino-5-chloropyrimidin-4-ylamino)-4-(morpholinomethyl)phenyl)-2-(thiazol-2-yl)but-3-yn-2-ol (163). After formation of the racemic mixture (prepared from components A.204 and C.4) using the analogous synthetic techniques as in example 143, the enantiomers were separated by chiral HPLC using an AD-H column with isocratic 15% isopropanol in hexanes. The first compound to elute off the column was assigned as (S)-4-(3-(2-amino-5-chloropyrimidin-4-ylamino)-4-(morpholinomethyl)phenyl)-2-(thiazol-2-yl)but-3-yn-2-ol (162).

The second compound to elute off the column was assigned as (R)-4-(3-(2-amino-5-chloropyrimidin-4-ylamino)-4-(morpholinomethyl)phenyl)-2-(thiazol-2-yl)but-3-yn-2-ol (163). Stereochemistry is assigned based on bioactivity and analogy to other active isomers where the stereocenter was confirmed by crystallography.

(R)-4-(3-(2-amino-5-chloropyrimidin-4-ylamino)-4-(morpholinomethyl)phenyl)-2-(thiazol-2-yl)but-3-yn-2-ol (163) (20 mg). ¹H NMR (500 MHz, DMSO-d₆) δ ppm 9.88 (1H, s), 8.21 (1H, s), 7.99 (1H, s), 7.76 (1H, d, J=3.2 Hz), 7.68 (1H, d, J=3.2 Hz), 7.24 (1H, d, J=7.8 Hz), 7.06 (1H, m), 6.97 (1H, s), 6.47 (2H, s), (6H, m), 2.38 (4H, m), 1.88 (3H, s). MS ESI (pos.) m/e: 471.1 (M+H).

### Example 165

Examples 165-175 were prepared using the same or analogous synthetic techniques and substituting with appropriate reagents as for example 143.

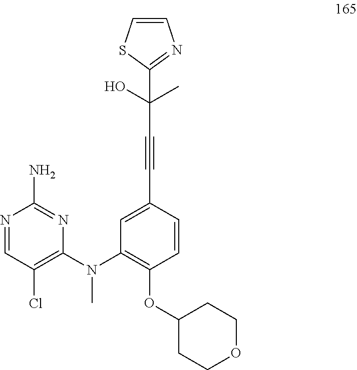

4-(3-((2-amino-5-chloropyrimidin-4-yl)(methyl)amino)-4-(tetrahydro-2H-pyran-4-yloxy)phenyl)-2-(thiazol-2-yl)but-3-yn-2-ol (165) (prepared from components A.210 and C.4). ¹H NMR (500 MHz, DMSO-d₆) δ ppm 7.76 (2H, m), 7.66 (1H, d, J=2.9 Hz), 7.27 (2H, m), 7.07 (1H, d, J=8.6 Hz), 6.96 (1H, s), 6.42 (2H, s), 4.62 (1H, dd, J=7.1, 3.4 Hz), 3.62 (2 H, m), 3.43 (2H, ddd, J=11.2, 7.5, 3.3 Hz), 3.24 (3H, s), 1.86 (5H, m), 1.44 (2H, m). MS ESI (pos.) m/e: 486.1 (M+H).

### Example 166

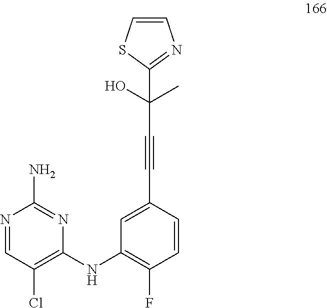

4-(3-(2-Amino-5-chloropyrimidin-4-ylamino)-4-fluorophenyl)-2-(thiazol-2-yl)but-3-yn-2-ol (166) (prepared from components A.212 and C.4). ¹H NMR (500 MHz, DMSO-d₆) δ ppm 8.46 (1H, m), 7.93 (1H, m), 7.78 (1H, m), 7.69 (1H, ms), 7.54 (1H, d, J=6.4 Hz), 7.34 (2H, m), 7.06 (1H, m), 6.34 (2H, s), 1.88 (3H, s). MS ESI (pos.) m/e: 390.1 (M+H).

### Example 167

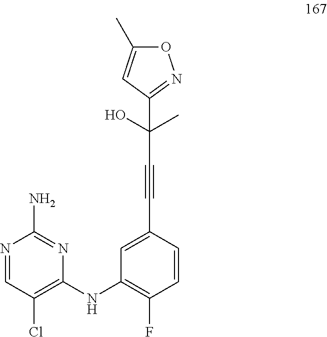

4-(3-(2-Amino-5-chloropyrimidin-4-ylamino)-4-fluorophenyl)-2-(5-methylisoxazol-3-yl)but-3-yn-2-ol (167) (prepared from components A.212 and C.1). ¹H NMR (500 MHz, DMSO-d₆) δ ppm 8.44 (1H, m), 7.92 (1H, m), 7.55 (1H, d, J=7.3 Hz), 7.28 (2H, d, J=8.6 Hz), 6.48 (1H, m), 6.34 (3H, d, J=11.0 Hz), 2.40 (3H, s), 1.78 (3H, s). MS ESI (pos.) m/e: 388.1 (M+H).

### Example 168

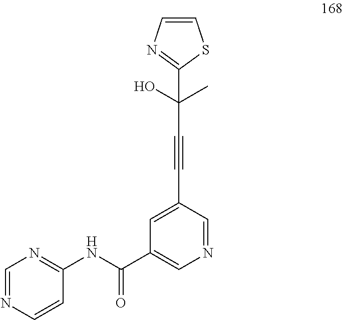

5-(3-Hydroxy-3-(thiazol-2-yl)but-1-ynyl)-N-(pyrimidin-4-yl)nicotinamide (168) (prepared from components A.215 and C.4). ¹H NMR (500 MHz, DMSO-d₆) δ ppm 11.54 (1H, s), 9.07 (1H, d, J=2.0 Hz), 8.97 (1H, s), 8.80 (1H, d, J=2.0 Hz), 8.75 (1H, d, J=5.6 Hz), 8.42 (1H, d, J=2.0 Hz), 8.19 (1H, d, J=5.9 Hz), 7.79 (1H, d, J=3.2 Hz), 7.71 (1H, d, J=3.2 Hz), 7.19 (1H, s), 1.91 (3H, s). MS ESI (pos.) m/e: 352.0 (M+H).

### Example 169

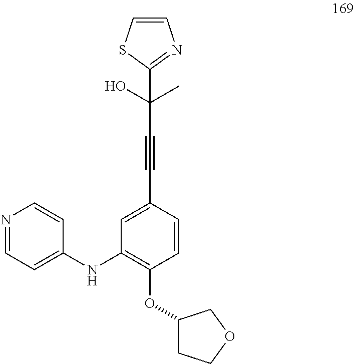

4-(3-(Pyridin-4-ylamino)-4-(S)-tetrahydrofuran-1-yloxy)phenyl)-2-(thiazol-2-yl)but-3-yn-2-ol (169). To a flask containing (S)-N-(5-iodo-2-(tetrahydrofuran-3-yloxy)phenyl)pyridin-4-amine A.217 (100.0 mg, 0.26 mmol), 2-(thiazol-2-yl)but-3-yn-2-ol C.4 (160.8 mg, 1.05 mmol), copper (I) iodide (10.0 mg, 0.053 mmol), and PdCl₂(PPh₃)₂ (18.5 mg, 0.026 mmol) was added dry DMF (2.00 mL) and triethylamine (2.00 mL). The mixture was heated to 100° C. After 2.5 hours, the solvent was removed, then the residue was purified by silica gel flash chromatography (25:1:1 solution of dichloromethane: methanol: 2M ammonia in methanol) to afford 4-(3-(pyridin-4-ylamino)-4-(S)-tetrahydrofuran-3-yloxy)phenyl)-2-(thiazol-2-yl)but-3-yn-2-ol (169) (77 mg, 72%). MS ESI (pos.) m/e: 408.1 (M+H).

### Example 170

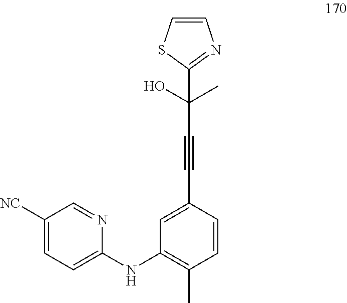

6-(5-(3-Hydroxy-3-(thiazol-2-yl)but-1-ynyl)-2-methylphenylamino)nicotinonitrile (170) (prepared from components A.219 and C.4). ¹H NMR (500 MHz, DMSO-d₆) δ ppm 9.10 (1H, s), 8.48 (1H, d, J=2.4 Hz), 7.86 (1H, dd, J=8.8, 2.2 Hz), 7.76 (1H, d, J=3.2 Hz), 7.67 (1H, d, J=3.2 Hz), 7.52 (1H, s), 7.26 (1H, d, J=7.8 Hz), 7.14 (1H, dd, J=7.8, 1.5 Hz), 7.00 (1H, s), 6.80 (1H, d, J=8.8 Hz), 2.24 (3H, m), 1.88 (3H, m). MS ESI (pos.) m/e: 361.1 (M+H).

### Example 171

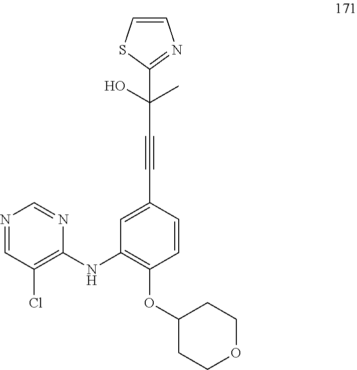

4-(3-(5-Chloropyrimidin-4-ylamino)-4-(tetrahydro-2H-pyran-4-yloxy)phenyl)-2-(thiazol-2-yl)but-3-yn-2-ol (171) (prepared from components A.220 and C.4). ¹H NMR (500 MHz, DMSO-d₆) δ ppm 8.61 (1H, s), 8.54 (1H, s), 8.42 (1H, s), 8.18 (1H, s), 7.77 (1H, d, J=3.2 Hz), 7.68 (1H, d, J=3.2 Hz), 7.20 (2H, s), 7.00 (1H, s), 4.75 (1H, dt, J=7.6, 3.9 Hz), 3.79 (2 H, m), 3.50 (2H, ddd, J=11.6, 8.2, 3.2 Hz), 2.00 (2H, m), 1.87 (3H, s), 1.64 (2H, m). MS ESI (pos.) m/e: 457.1 (M+H).

### Example 172

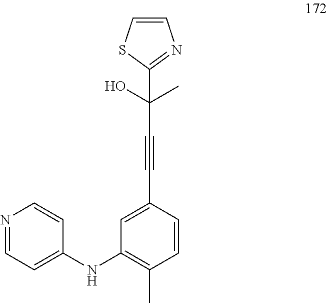

4-(4-Methyl-3-(pyridin-4-ylamino)phenyl)-2-(thiazol-2-yl)but-3-yn-2-ol (172) (prepared from components A.221 and C.4). ¹H NMR (500 MHz, DMSO-d₆) δ ppm 8.42 (1H, s), 8.15 (2H, d, J=5.1 Hz), 7.75 (1H, d, J=3.2 Hz), 7.67 (1H, d, J=3.2 Hz), 7.31 (1H, d, J=7.8 Hz), 7.22 (1H, s), 7.16 (1H, d, J=7.6 Hz), 6.99 (1H, s), 6.64 (2H, d, J=5.4 Hz), 2.19 (3H, s), 1.85 (3H, s). MS ESI (pos.) m/e: 336.1 (M+H).

### Example 173

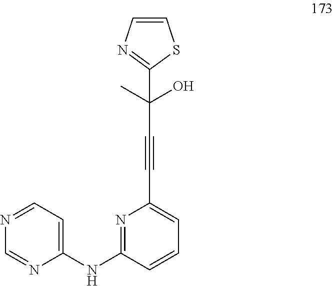

4-(6-(pyrimidin-4-ylamino)pyridin-2-yl)-2-(thiazol-2-yl)but-3-yn-2-ol (173) (prepared from components A.223 and C.4). ¹H NMR (500 MHz, DMSO-d₆) δ ppm 10.39 (1H, s), 8.69 (1H, s), 8.39 (1H, d, J=5.9 Hz), 7.92 (1H, d, J=8.6 Hz), 7.80 (2H, m), 7.69 (1H, m), 7.45 (1H, d, J=5.9 Hz), 7.17 (2H, m), 1.93 (3H, m). MS ESI (pos.) m/e: 324.0 (M+H).

### Example 174

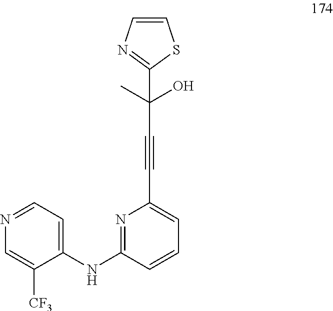

2-(thiazol-2-yl)-4-(6-(3-(trifluoromethyl)pyridin-4-ylamino)pyridin-2-yl)but-3-yn-2-ol (174) (prepared from components A.225 and C.4). ¹H NMR (500 MHz, DMSO-d₆) δ ppm 8.72 (2H, m), 8.56 (1H, d, J=5.9 Hz), 7.94 (1H, d, J=5.9 Hz), 7.78 (2H, m), 7.70 (1H, m), 7.29 (1H, d, J=8.3 Hz), 7.16 (2H, m), 1.86 (3H, s). MS ESI (pos.) m/e: 391.0 (M+H).

### Example 175

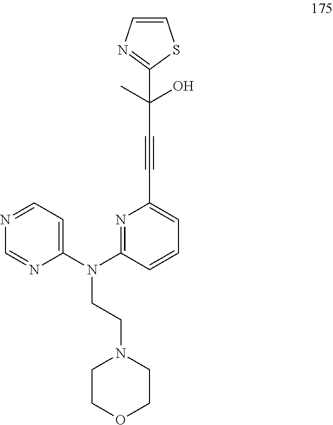

4-(6-((2-morpholinoethyl)(pyrimidin-4-yl)amino)pyridin-2-yl)-2-(thiazol-2-yl)but-3-yn-2-ol (175) (prepared from components A.226 and C.4). ¹H NMR (500 MHz, DMSO-d₆) δ ppm 8.68 (1H, s), 8.28 (1H, d, J=6.1 Hz), 7.87 (1H, m), 7.78 (1H, dd, J=3.2, 1.0 Hz), 7.70 (1H, dd, J=3.2, 1.0 Hz), 7.48 (1H, d, J=8.3 Hz), 7.35 (1H, d, J=7.6 Hz), 7.16 (1H, s), 6.84 (1H, d, J=6.1 Hz), 4.19 (2H, t, J=6.5 Hz), 3.42 (4H, m), 2.54 (2H, m), 2.32 (4H, m), 1.88 (3H, s). MS ESI (pos.) m/e: 437.0 (M+H).

### Example 176 and 177

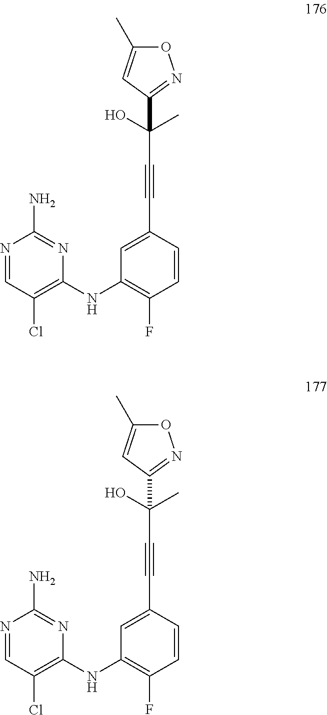

(R)-4-(3-(2-Amino-5-chloropyrimidin-4-ylamino)-4-fluorophenyl)-2-(5-methylisoxazol-3-yl)but-3-yn-2-ol (176) and (S)-4-(3-(2-amino-5-chloropyrimidin-4-ylamino)-4-fluorophenyl)-2-(5-methylisoxazol-3-yl)but-3-yn-2-ol (177). After formation of the racemic mixture prepared from components A.210 and C.1 using the analogous synthetic techniques as in example 143, the enantiomers were separated by chiral HPLC using an OJ-H column with isocratic 15% isopropanol in hexanes. The first compound to elute off the column was assigned as (R)-4-(3-(2-amino-5-chloropyrimidin-4-ylamino)-4-fluorophenyl)-2-(5-methylisoxazol-3-yl)but-3-yn-2-ol (176), based on bioactivity and analogy to other active isomers where the stereocenter was confirmed by crystallography. (R)-4-(3-(2-amino-5-chloropyrimidin-4-ylamino)-4-fluorophenyl)-2-(5-methylisoxazol-3-yl)but-3-yn-2-ol (176).

¹H NMR (500 MHz, DMSO-d₆) δ ppm 8.45 (1H, s), 7.93 (1H, s), 7.57 (1H, d, J=7.3 Hz), 7.30 (2H, s), 6.49 (1H, s), 6.35 (3H, d, J=10.8 Hz), 2.41 (3H, s), 1.79 (3H, s). MS ESI (pos.) m/e: 388.1 (M+H).

### Example 178 and 179

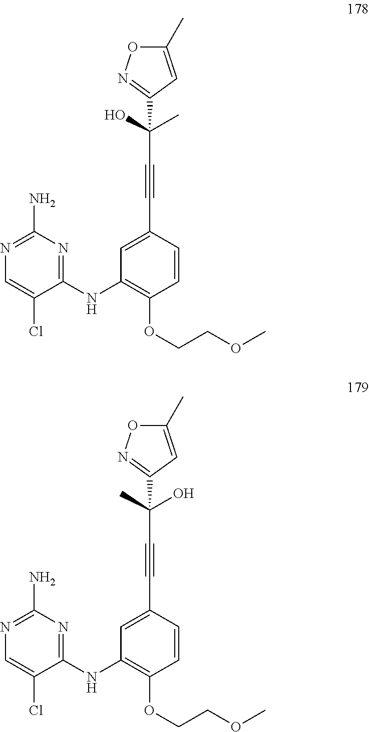

### (S)-4-(3-(2-amino-5-chloropyrimidin-4-ylamino)-4-(2-methoxyethoxy)phenyl)-2-(5-methylisoxazol-3-yl)but-3-yn-2-ol (178), and (R)-4-(3-(2-amino-5-chloropyrimidin-4-ylamino)-4-(2-methoxyethoxy)phenyl)-2-(5-methylisoxazol-3-yl)but-3-yn-2-ol (179)

After formation of the racemic mixture (0.30 g) prepared from components A.229 and C.1 using the analogous synthetic techniques as in example 143, the enantiomers were separated on a preparative chiral column (OD-H Chiralpak, 250×20 mm) using 8% isopropanol in hexane.

Stereochemistry is based on bioactivity and analogy to other active isomers where the stereocenter was confirmed by crystallography The first eluted enantiomer, assigned as (S)-4-(3-(2-amino-5-chloropyrimidin-4-ylamino)-4-(2-methoxyethoxy)phenyl)-2-(5-methylisoxazol-3-yl)but-3-yn-2-ol (178), was obtained as a light yellow solid (0.0231 g).; ¹H NMR (500 MHz, DMSO-d₆) δ ppm 8.34 (1H, d, J=1.5 Hz), 7.87-8.03 (2H, m), 7.04-7.17 (2H, m), 6.57 (2H, s), 6.38 (2H, d, J=16.6 Hz), 4.21-4.25 (2H, m), 3.66-3.69 (2H, m), 3.31 (3H, s), 2.37-2.43 (3H, m), 1.80 (3H, s); Mass Spectrum (ESI) m/e=444.1 [M+1].

The second eluted enantiomer, assigned as (R)-4-(3-(2-amino-5-chloropyrimidin-4-ylamino)-4-(2-methoxyethoxy)phenyl)-2-(5-methylisoxazol-3-yl)but-3-yn-2-ol (179) was obtained as a tan solid (0.0356 g): ¹H NMR (400 MHz, DMSO-d₆) δ ppm 8.34 (1H, d, J=1.6 Hz), 7.92-8.00 (2H, m), 7.07-7.13 (2H, m), 6.58 (2H, s), 6.35-6.42 (2H, m), 4.18-4.26 (2H, m), 3.63-3.71 (2H, m), 3.30 (3H, s), 2.40 (3H, s), 1.80 (3H, s); Mass Spectrum (ESI) m/e=444.1 [M+1].

### Example 180

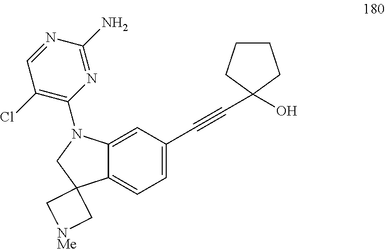

1-((1′-(2-Amino-5-chloro-4-pyrimidinyl)-1-methyl-1′,2′-dihydrospiro[azetidine-3,3′-indol]-6′-yl)ethynyl)cyclonentanol

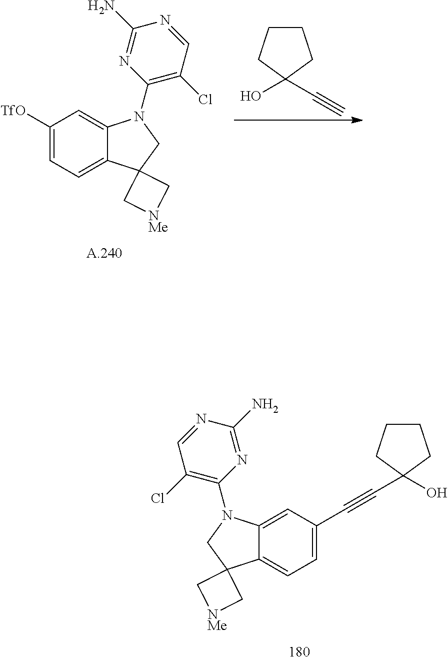

1-((1′-(2-amino-5-chloropyrimidin-4-yl)-1-methylspiro[azetidine-3,3′-indoline]-6′-yl)ethynyl)cyclopentanol (180) was prepared in analogy to the procedure of Alami, M., F. Ferri, et al. (1993). Tetrahedron Lett. 34(40): 6403-6406.

To a 50 mL single-necked round bottom flask were placed 1′-(2-amino-5-chloropyrimidin-4-yl)-1-methylspiro[azetidine-3,3′-indoline]-6′-yltrifluoromethanesulfonate A.240 (0.18 g, 0.4 mmol) and Pd(PPh₃)₄ (10 mol %, 0.046 mg). The flask was subjected to 3 cycles of evacuation and back-filling with N₂. Piperidine (10 mL) was added followed by 1-ethynylcyclopentanol C.36 (5.0 eq., 0.23 mL). The resulting mixture was stirred under N₂ at 80° C. for 1.5 h. Upon workup, the volatiles were removed in vacuo and the residue was subjected to combi-flash column chromatography (methanol/dichloromethane with triethylamine) to give 1-((1′-(2-amino-5-chloropyrimidin-4-yl)-1-methylspiro[azetidine-3,3′-indoline]-6′-yl)ethynyl)cyclopentanol (180) (0.10 g, 70% yield) as an off-white solid. ¹H NMR (400 MHz, DMSO-d₆) δ ppm 8.12 (1H, s), 7.60 (1H, d, J=8.2 Hz), 7.26 (1H, s), 7.05 (1H, d, J=7.4 Hz), 6.66 (2H, s), 5.25 (1H, s), 4.41 (2H, s), 3.43-3.77 (4H, m), 3.30 (3H, s), 1.80-1.95 (4H, m), 1.59-1.79 (4H, m). LCMS-ESI (POS), M/Z, M+1: Found 410.0.

### Example 181

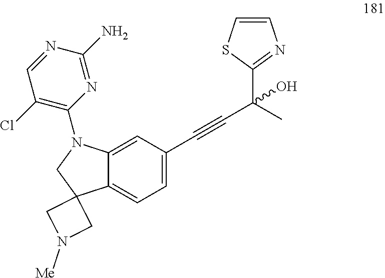

(rac)-4-(1′-(2-amino-5-chloropyrimidin-4-yl)-1-methylspiro[azetidine-3,3′-indoline]-6′-yl)-2-(thiazol-2-yl)but-3-yn-2-ol (181) was prepared from 1′-(2-amino-5-chloropyrimidin-4-yl)-1-methylspiro[azetidine-3,3′-indoline]-6′-yl trifluoromethanesulfonate A.240 and (rac)-2-(1,3-thiazol-2-yl)-3-butyn-2-ol C.4 using chemistry similar to that described in Example 180. (an off-white solid) ¹H NMR (400 MHz, DMSO-d₆) δ ppm 8.11 (1H, s), 7.76 (1H, d, J=3.5 Hz), 7.66 (1H, d, J=3.1 Hz), 7.55 (1H, d, J=7.4 Hz), 7.25 (1H, d, J=1.2 Hz), 7.05 (1H, dd, J=7.8, 1.2 Hz), 6.95 (1H, s), 6.64 (2H, br. s.), 4.35 (2H, s), 3.35-3.39 (2H, m), 3.32-3.34 (2H, m), 2.28 (3H, s), 1.86 (3H, s). LCMS-ESI (POS), M/Z, M+1: Found 453.1.

### Example 182

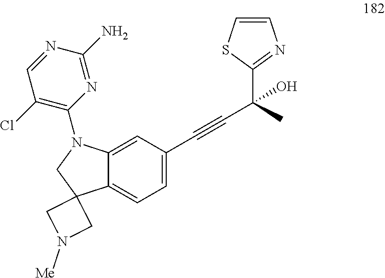

### (2R*)-4-(1′-(2-Amino-5-chloro-4-pyrimidinyl)-1-methyl-1′,2′-dihydrospiro[azetidine-3,3′-indol]-6′-yl)-2-(1,3-thiazol-2-yl)-3-butyn-2-ol

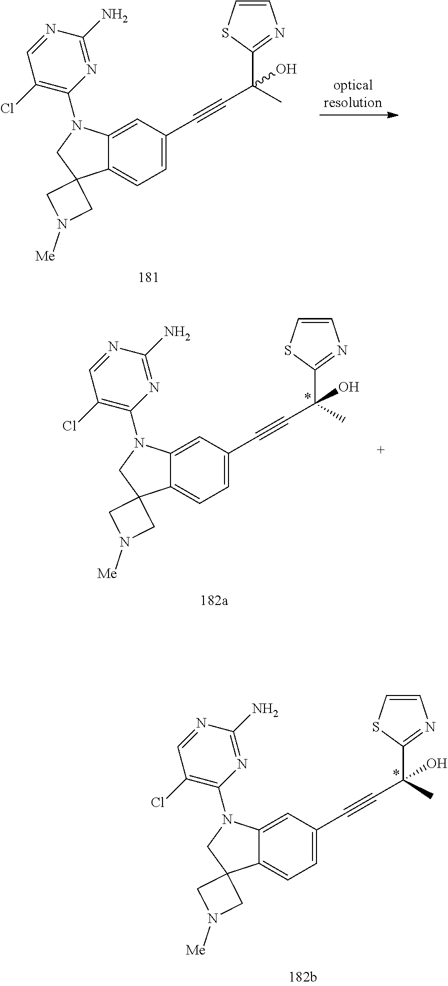

### (2S*)-4-(1′-(2-Amino-5-chloro-4-pyrimidinyl)-1-methyl-1′,2′-dihydrospiro[azetidine-3,3′-indol]-6′-yl)-2-(1,3-thiazol-2-yl)-3-Butyn-2-ol (12) (182a) and (2R*)-4-(1′-(2-Amino-5-chloro-4-pyrimidinyl)-1-methyl-1′,2′-dihydrospiro[azetidine-3,3′-indol]-6′-yl)-2-(1,3-thiazol-2-yl)-3-butyn-2-ol (182b)

Optical resolution of the racemic mixture of example 181 was accomplished by the following method. Instrument: Agilent 1100 series. Column: AD-preparative (50 mm×500 mm). Solvents: 15% isopropanol in hexanes. Gradient: isochratic. Separation quality: close to baseline separation. Stereochemistry is assigned by both biological activity and chiral HPLC elution order by analogy to defined compounds. The first peak off of the AD column provided (2 S)-4-(1′-(2-amino-5-chloro-4-pyrimidinyl)-1-methyl-1′,2′-dihydrospiro[azetidine-3,3′-indol]-6′-yl)-2-(1,3-thiazol-2-yl)-3-butyn-2-ol (182a) as an off-white solid. ¹H NMR (400 MHz, DMSO-d₆) δ ppm 8.11 (1H, s), 7.76 (1H, d, J=3.5 Hz), 7.66 (1H, d, J=3.1 Hz), 7.55 (1H, d, J=7.4 Hz), 7.25 (1H, d, J=1.2 Hz), 7.05 (1H, dd, J=7.8, 1.2 Hz), 6.95 (1H, s), 6.64 (2H, br. s.), 4.35 (2H, s), 3.35-3.39 (2H, m), 3.32-3.34 (2H, m), 2.28 (3H, s), 1.86 (3H, s). LCMS-ESI (POS), M/Z, M+1: Found 453.1.

The second eluting peak provided (2R)-4-(1′-(2-amino-5-chloro-4-pyrimidinyl)-1-methyl-1′,2′-dihydrospiro[azetidine-3,3′-indol]-6′-yl)-2-(1,3-thiazol-2-yl)-3-butyn-2-ol (182b) as an off-white solid. (96.5% ee) ¹H NMR (400 MHz, DMSO-d₆) δ ppm 8.11 (1H, s), 7.76 (1H, d, J=3.5 Hz), 7.66 (1H, d, J=3.1 Hz), 7.55 (1H, d, J=7.4 Hz), 7.25 (1H, d, J=1.2 Hz), 7.05 (1H, dd, J=7.8, 1.2 Hz), 6.95 (1H, s), 6.64 (2H, br. s.), 4.35 (2H, s), 3.35-3.39 (2H, m), 3.32-3.34 (2H, m), 2.28 (3H, s), 1.86 (3H, s). NMR. LCMS-ESI (POS), M/Z, M+1: Found 453.1.

### Example 183

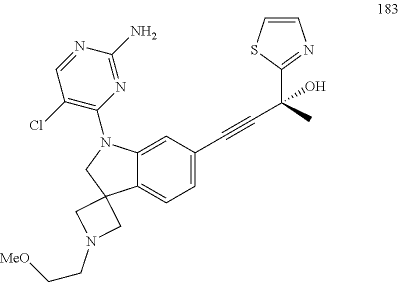

(2R)-4-(1′-(2-amino-5-chloropyrimidin-4-yl)-1-(2-methoxyethyl)spiro[azetidine-3,3′-indoline]-6′-yl)-2-(thiazol-2-yl)but-3-yn-2-ol (183) was prepared from components A.245 and A.6 using chemistry similar to that described in Example 180. ¹H NMR (400 MHz, DMSO-d₆) δ ppm 8.11 (1H, s), 7.76 (1H, d, J=3.1 Hz), 7.66 (1H, d, J=3.1 Hz), 7.55 (1H, d, J=7.8 Hz), 7.26 (1H, d, J=1.2 Hz), 7.05 (1H, dd, J=7.8, 1.2 Hz), 6.95 (1H, s), 6.64 (2H, br. s.), 4.35 (2H, s), 3.41 (2H, d, J=7.0 Hz), 3.31-3.35 (4H, m), 3.23 (3H, s), 2.61 (2H, t, J=5.9 Hz), 1.86 (2H, s). LCMS-ESI (POS), M/Z, M+1: Found 497.2.

### Example 184

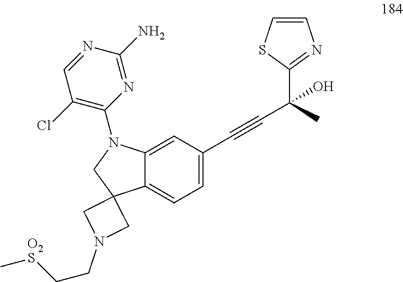

(R)-4-(1′-(2-amino-5-chloropyrimidin-4-yl)-1-(2-(methylsulfonyl)ethyl)spiro[azetidine-3,3′-indoline]-6′-yl)-2-(thiazol-2-yl)but-3-yn-2-ol (184) was prepared from components A.251 and C.6 using chemistry similar to that described in Example 180. (an off-white solid)

¹H NMR (400 MHz, CHLOROFORM-d) δ ppm 8.10 (1H, s), 7.78 (1H, d, J=3.1 Hz), 7.59 (1 H, s), 7.43 (1H, d, J=7.8 Hz), 7.35 (1H, d, J=3.1 Hz), 7.17 (1H, d, J=8.2 Hz), 4.94 (2H, s), 4.45 (2H, s), 3.71 (1H, br. s.), 3.43-3.55 (4H, m), 3.00-3.11 (7H, m), 2.05 (3H, s). LCMS-ESI (POS), M/Z, M+1: Found 545.0.

### Example 185

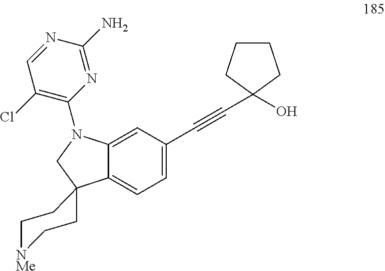

### 1-((1-(2-Amino-5-chloro-4-pyrimidinyl)-1′-methyl-1,2-dihydrospiro[indole-3,4′-piperidin]-6-yl)ethynyl)cyclopentanol (185)

To a 10 mL single-necked round bottom flask were placed 4-(6-bromo-1′-methylspiro[indoline-3,4′-piperidine]-1-yl)-5-chloropyrimidin-2-amine A.254 (0.10 g, 0.244 mmol), PdCl₂(PPh₃)₂ (20 mol %), and CuI (40 mol %). The flask was subjected to 3 cycles of evacuation and back-filling with N₂. DMSO (5 mL), triethylamine (1 mL), and 1-ethynylcyclopentanol C.36 (5.0 eq.) were added sequentially under N₂. The resulting mixture was heated at 95° C. for 14 h. Upon workup, the mixture was cooled and poured into ice and 2 N NaOH aqueous solution and extracted with ethyl acetate (2×). The combined organics were dried over Na₂SO₄ and concentrated in vacuo. The residue was subjected to combi-flash column chromatography (methanol/dichloromethane with triethylamine) to give 1-((1-(2-amino-5-chloro-4-pyrimidinyl)-1′-methyl-1,2-dihydrospiro[indole-3,4′-piperidin]-6-yl)ethynyl)cyclopentanol (185) as an off-white solid. ¹H NMR (400 MHz, DMSO-d₆) δ ppm 8.12 (1H, s), 7.20 (1H, d, J=7.7 Hz), 7.08 (1H, s), 6.95 (1H, d, J=8.1 Hz), 6.68 (2H, s), 5.25 (1H, s), 4.00 (2H, s), 2.63-2.86 (2H, m), 2.22 (3H, br. s.), 2.00-2.09 (2H, m), 1.79-1.94 (6H, m), 1.52-1.78 (6H, m). LCMS-ESI (POS), M/Z, M+1: Found 438.3.

Examples 186-197 were prepared using the procedure described in Example 185.

### Example 186

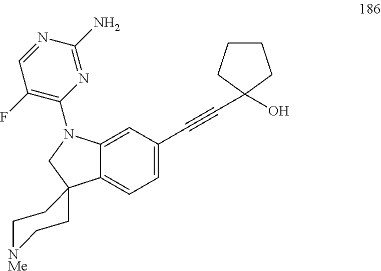

1-((1-(2-amino-5-fluoropyrimidin-4-yl)-1′-methylspiro[indoline-3,4′-piperidine]-6-yl)ethynyl)cyclopentanol(186) was prepared from components A.255 and C.36 as an off-white solid. ¹H NMR (500 MHz, DMSO-d₆) δ ppm 8.03 (1H, d, J=5.9 Hz), 7.79 (1H, s), 7.21 (1H, d, J=7.8 Hz), 6.99 (1H, dd, J=7.7, 1.3 Hz), 6.36 (2H, s), 5.25 (1H, s), 4.04 (2H, d, J=3.7 Hz), 2.74 (2H, d, J=11.5 Hz), 2.20 (3H, s), 1.79-2.01 (8H, m), 1.54-1.78 (6H, m). LCMS-ESI (POS), M/Z, M+1: Found 422.2.

### Example 187

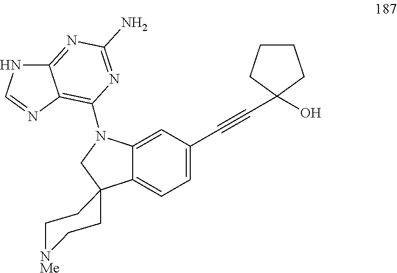

1-((1-(2-amino-9H-purin-6-yl)-1′-methylspiro[indoline-3,4′-piperidine]-6-yl)ethynyl)cyclopentanol (187) was prepared from components A.257 and C.36 as an off-white solid. ¹H NMR (400 MHz, DMSO-d₆) δ ppm 8.56 (1H, s), 7.86 (1H, s), 7.21 (1H, d, J=7.8 Hz), 6.95-7.05 (1H, m), 6.01 (2H, s), 5.24 (1H, br. s.), 4.60 (2H, s), 2.73-2.86 (2H, m), 2.23 (3H, s), 1.82-2.09 (8H, m), 1.60-1.80 (6H, m). LCMS-ESI (POS), M/Z, M+1: Found 444.2.

### Example 188

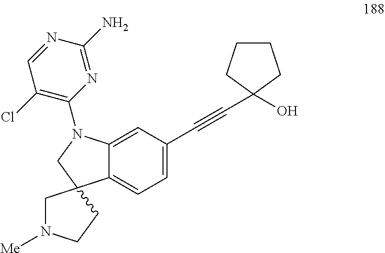

1-((1-(2-Amino-5-chloro-4-pyrimidinyl)-r-methyl-1,2-dihydrospiro[indole-3,3′-pyrrolidin]-6-yl)ethynyl)cyclopentanol (188) was prepared from components A.261 and C.36 as. ¹H NMR (400 MHz, DMSO-d₆) δ ppm 8.11 (1H, s), 7.26 (1H, d, J=7.7 Hz), 7.16 (1 H, d, J=1.1 Hz), 6.97 (1H, dd), 6.65 (2H, s), 5.25 (1H, s), 4.11 (1H, d), 4.03 (1H, d, J=10.6 Hz), 2.74 (1H, td, J=8.3, 5.7 Hz), 2.67 (1H, d, J=9.1 Hz), 2.52-2.59 (1H, m), 2.52-2.58 (2 H, m), 2.42 (1H, d, J=9.1 Hz), 2.27 (3H, s), 1.98-2.12 (2H, m), 1.80-1.94 (4H, m), 1.59-1.77 (4H, m). LCMS-ESI (POS), M/Z, M+1: Found 424.1.

### Example 189

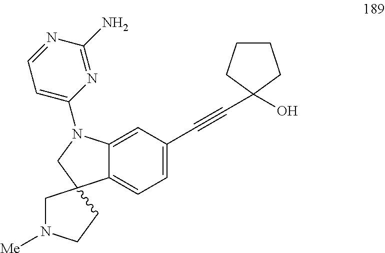

(rac)-1-((1-(2-Amino-4-pyrimidinyl)-1′-methyl-1,2-dihydrospiro[indole-3,3′-pyrrolidin]-6-yl)ethynyl)cyclopentanol (189) was prepared from components A.262 and C.36 as an off-white solid. ¹H NMR (500 MHz, DMSO-d₆) δ ppm 8.34 (1H, s), 7.97 (1H, d, J=5.9 Hz), 7.27 (1H, d, J=7.3 Hz), 6.99 (1H, d, J=7.8 Hz), 6.35 (2H, s), 6.06 (1H, d, J=5.9 Hz), 5.25 (1H, br. s.), 3.96 (1H, d), 3.87 (1H, d, J=10.3 Hz), 2.72-2.82 (1H, m), 2.68 (1H, d, J=9.3 Hz), 2.55-2.62 (1H, m), 2.47 (1H, d, J=9.3 Hz), 2.30 (3H, s), 2.00-2.14 (2H, m), 1.84-1.96 (4 H, m), 1.61-1.81 (4H, m). LCMS-ESI (POS), M/Z, M+1: Found 390.2.

### Example 190

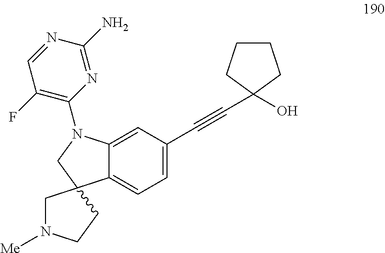

(rac)-1-((1-(2-Amino-5-fluoro-4-pyrimidinyl)-1′-methyl-1,2-dihydrospiro[indole-3,3′-pyrrolidin]-6-yl)ethynyl)cyclopentanol (190) was prepared from components A.263 and C.36 as an off-white solid. ¹H NMR (400 MHz, DMSO-d₆) δ ppm 8.02 (1H, d, J=5.9 Hz), 7.86 (1H, s), 7.28 (1H, d, J=7.7 Hz), 7.01 (1H, dd, J=7.7, 1.5 Hz), 6.34 (2H, s), 5.25 (1H, s), 4.18 (1H, dd, J=10.8, 3.8 Hz), 4.07 (1H, dd, J=10.8, 3.8 Hz), 2.67-2.80 (2H, m), 2.53-2.60 (1H, m), 2.44 (1H, d, J=9.1 Hz), 2.28 (3H, s), 2.04-2.10 (2H, m), 1.85-1.95 (4H, m), 1.64-1.78 (4H, m). LCMS-ESI (POS), M/Z, M+1: Found 408.3.

### Example 191

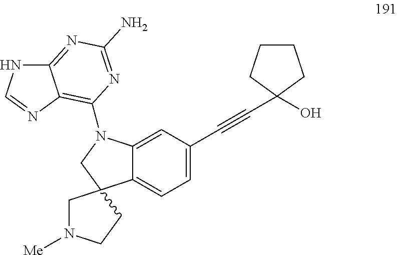

1-((1-(2-Amino-9H-purin-6-yl)-1′-methyl-1,2-dihydrospiro[indole-3,3′-pyrrolidin]-6-yl)ethynyl)cyclopentanol (191) was prepared from components A.264 and C.36 as an off-white solid. ¹H NMR (400 MHz, DMSO-d₆) δ ppm 12.36 (1H, br. s.), 8.54 (1H, s), 7.80 (1H, s), 7.29 (1H, d, J=7.7 Hz), 7.02 (1H, dd, J=7.9, 1.3 Hz), 6.04 (2H, s), 5.24 (1H, s), 4.80 (1H, d, J=11.7 Hz), 4.52 (1H, d, J=12.1 Hz), 2.78-2.88 (1H, m), 2.72 (1H, d, J=9.1 Hz), 2.53-2.61 (1H, m), 2.46 (1H, d, J=9.1 Hz), 2.30 (3H, s), 2.01-2.20 (2H, m), 1.88-1.96 (4H, m), 1.60-1.82 (4H, m). LCMS-ESI (POS), M/Z, M+1: Found 430.1.

### Example 192

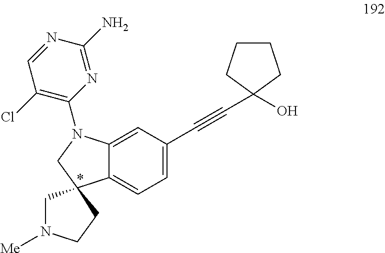

1-(((3S*)-1-(2-Amino-5-chloro-4-pyrimidinyl)-1′-methyl-1,2-dihydrospiro[indole-3,3′-pyrrolidin]-6-yl)ethynyl)cyclopentanol (192) was prepared from components A.267 and C.36 . ¹H NMR (400 MHz, DMSO-d₆) δ ppm 8.11 (1H, s), 7.26 (1H, d, J=7.7 Hz), 7.16 (1H, d, J=1.1 Hz), 6.97 (1H, dd), 6.65 (2H, s), 5.25 (1H, s), 4.11 (1H, d), 4.03 (1H, d, J=10.6 Hz), 2.74 (1H, td, J=8.3, 5.7 Hz), 2.67 (1H, d, J=9.1 Hz), 2.52-2.59 (1H, m), 2.52-2.58 (2 H, m), 2.42 (1H, d, J=9.1 Hz), 2.27 (3H, s), 1.98-2.12 (2H, m), 1.80-1.94 (4H, m), 1.59-1.77 (4H, m). LCMS-ESI (POS), M/Z, M+1: Found 424.1.

### Example 193

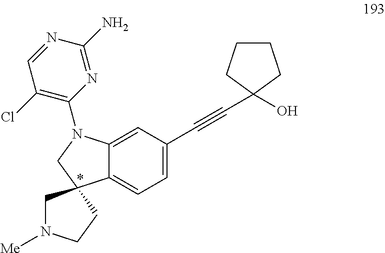

1-(((3R*)-1-(2-Amino-5-chloro-4-pyrimidinyl)-1′-methyl-1,2-dihydrospiro[indole-3,3′-pyrrolidin]-6-yl)ethynyl)cyclopentanol (193) was prepared from components A.266 and C.36. (enantiomeric with Example 192).

### Example 194

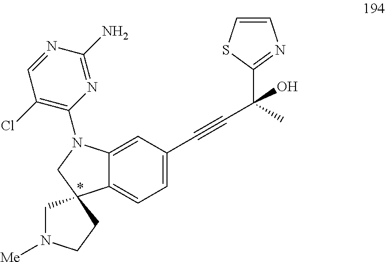

### (2S)-4-((3R*)-1-(2-Amino-5-chloro-4-pyrimidinyl)-1′-methyl-1,2-dihydrospiro[indole-3,3′-pyrrolidin]-6-yl)-2-(1,3-thiazol-2-yl)-3-butyn-2-ol (194)

### Example 195

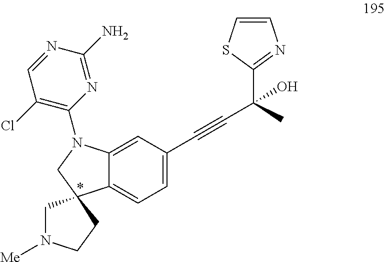

(2R)-4-((3R*)-1-(2-Amino-5-chloro-4-pyrimidinyl)-1′-methyl-1,2-dihydrospiro[indole-3,3′-pyrrolidin]-6-yl)-2-(1,3-thiazol-2-yl)-3-butyn-2-ol (195)

Resolution of the diastereomeric mixture prepared from components A.267 and C.4 using the procedure described in Example 185, was accomplished by the following method.

Instrument: Agilent 1100 series. Column: AD-preparative (50 mm×500 mm). Solvents: 17% isopropanol in hexanes. Gradient: isochratic. Separation quality: no baseline separation. Stereochemistry at the carbinol center was assigned by biological activity and relative elution order compared to compounds of established structure. The first peak to elute provided (2S)-4-((3R*)-1-(2-Amino-5-chloro-4-pyrimidinyl)-1′-methyl-1,2-dihydrospiro[indole-3,3′-pyrrolidin]-6-yl)-2-(1,3-thiazol-2-yl)-3-butyn-2-ol (194) as an off-white solid in a diastereomeric ratio of 99:1. ¹H NMR (400 MHz, DMSO-d₆) δ ppm 8.11 (1H, s), 7.74-7.77 (1H, m), 7.66 (1H, d, J=3.9 Hz), 7.28 (1H, d, J=7.4 Hz), 7.17 (1H, s), 7.00 (1H, d, J=8.2 Hz), 6.94 (1H, s), 6.65 (2H, s), 4.11 (1H, d), 4.04 (1H, d, J=10.6 Hz), 2.69-2.78 (1H, m), 2.67 (1H, d, J=9.0 Hz), 2.53-2.58 (1H, m), 2.42 (1H, d, J=9.0 Hz), 2.27 (3H, s), 1.98-2.11 (2H, m), 1.85 (3H, s). LCMS-ESI (POS), M/Z, M+1: Found 467.0.

The second peak furnished (2R)-4-((3R*)-1-(2-Amino-5-chloro-4-pyrimidinyl)-1′-methyl-1,2-dihydrospiro[indole-3,3′-pyrrolidin]-6-yl)-2-(1,3-thiazol-2-yl)-3-butyn-2-ol (195) as an off-white solid in a diastereomeric ratio of 2:98. ¹H NMR (400 MHz, DMSO-d₆) δ ppm 8.11 (1H, s), 7.76 (1H, d, J=3.1 Hz), 7.66 (1H, d, J=3.1 Hz), 7.28 (1H, d, J=7.8 Hz), 7.17 (1H, d, J=1.2 Hz), 7.00 (1H, dd, J=7.6, 1.4 Hz), 6.94 (1H, s), 6.65 (2H, s), 4.11 (1H, d), 4.04 (1H, d), 2.69-2.78 (1H, m), 2.66 (1H, d, J=8.6 Hz), 2.53-2.58 (1H, m), 2.43 (1H, d, J=9.0 Hz), 2.27 (3H, s), 1.96-2.13 (2H, m), 1.85 (3H, s). LCMS-ESI (POS), M/Z, M+1: Found 467.0.

### Example 196

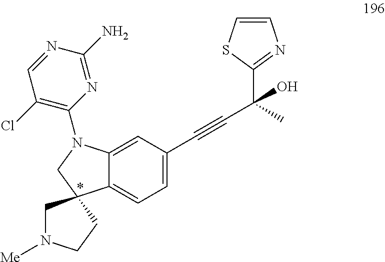

### (2S)-4-((3S*)-1-(2-Amino-5-chloro-4-pyrimidinyl)-1′-methyl-1,2-dihydrospiro[indole-3,3′-pyrrolidin]-6-yl)-2-(1,3-thiazol-2-yl)-3-butyn-2-ol (196)

### Example 197

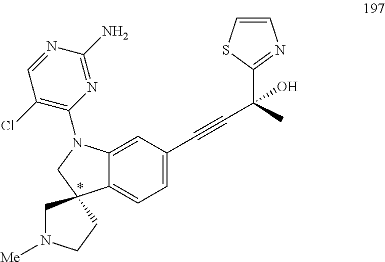

### (2R)-4-((3S*)-1-(2-Amino-5-chloro-4-pyrimidinyl)-1′-methyl-1,2-dihydrospiro[indole-3,3′-pyrrolidin]-6-yl)-2-(1,3-thiazol-2-yl)-3-butyn-2-ol (197)

Resolution of the diastereomeric mixture prepared from components A.257 and C.36 using the procedure described in Example 185, was accomplished by the following method. Instrument: Agilent 1100 series. Column: OD-preparative (50 mm×500 mm). Solvents: 9% isopropanol in hexanes. Gradient: isochratic. Separation quality: no baseline separation. The first peak to elute provided pure (2S)-4-((3 S *)-1-(2-amino-5-chloro-4-pyrimidinyl)-1′-methyl-1,2-dihydro spiro[indole-3 ,3′-pyrrolidin]-6-yl)-2-(1,3-thiazol-2-yl)-3-butyn-2-ol (196) as an off-white solid.

The second peak furnished (2R)-4-((3S*)-1-(2-Amino-5-chloro-4-pyrimidinyl)-1′-methyl-1,2-dihydrospiro[indole-3 ,3′-pyrrolidin]-6-yl)-2-(1,3-thiazol-2-yl)-3-butyn-2-ol (197) as an off-white solid in a diastereomeric ratio of (1.2:98.8). Stereochemistry at the carbinol center assigned by biological activity and analogy to compounds of established structure.

Examples 198-197 were prepared using the procedure described in Example 180.

### Example 198

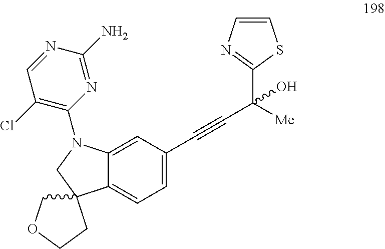

4-(1′-(2-Amino-5-chloro-4-pyrimidinyl)-1′,2′,4,5-tetrahydrospiro[furan-3,3′-indol]-6′-yl)-2-(1,3-thiazol-2-yl)-3-butyn-2-ol was prepared from components A.276 and C.4.

¹H NMR (400 MHz, DMSO-d₆) (signals for the mixture of 2 racemic diastereoisomers) δ ppm 8.13 (1H, s), 7.76 (1H, d, J=3.1 Hz), 7.66 (1H, d, J=3.1 Hz), 7.28 (1H, d, J=7.8 Hz), 7.18 (1 H, s), 7.00 (1H, d, J=7.8 Hz), 6.95 (1H, s), 6.67 (2H, s), 4.08-4.16 (2H, m), 3.94-4.02 (1 H, m), 3.84-3.93 (1H, m), 3.79 (1H, d, J=8.2 Hz), 3.65 (1H, d, J=8.6 Hz), 2.12-2.22 (2H, m), 1.86 (3H, s). LCMS-ESI (POS), M/Z, M+1: Found 454.1.

### Example 199

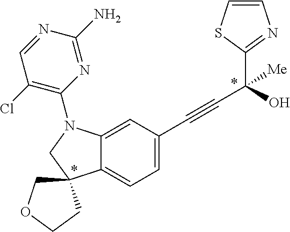

(2S)-4-((3R*)-1′-(2-Amino-5-chloro-4-pyrimidinyl)-1′,2′,4,5-tetrahydrospiro[furan-3,3′-indol]-6′-yl)-2-(1,3-thiazol-2-yl)-3-butyn-2-ol

### Example 200

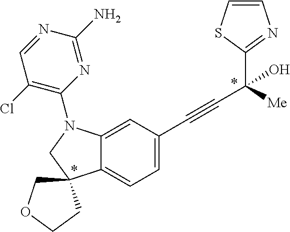

### (2R)-4-((3R*)-1′-(2-Amino-5-chloro-4-pyrimidinyl)-1′,2′,4,5-tetrahydrospiro[furan-3,3′-indol]-6′-yl)-2-(1,3-thiazol-2-yl)-3-butyn-2-ol

The diastereomeric mixture (2RS)-4-((3R*)-1′-(2-Amino-5-chloro-4-pyrimidinyl)-1′,2′,4,5-tetrahydrospiro[furan-3,3′-indol]-6′-yl)-2-(1,3-thiazol-2-yl)-3-butyn-2-ol (A.279) was prepared from components A.277 and C.4 by the procedure of example 180.

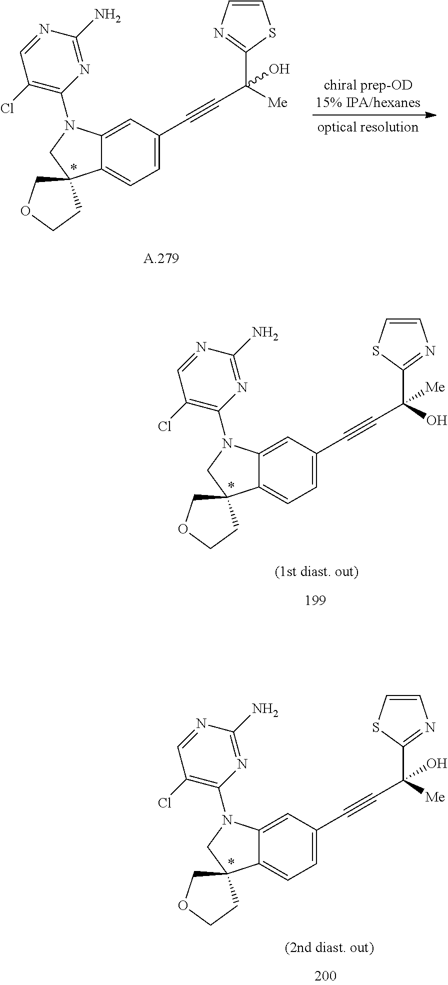

Resolution of A.279 was accomplished by the following method.

Instrument: Agilent 1100 series. Column: OD-preparative (50 mm×500 mm). Solvents: 15% isopropanol in hexanes. Gradient: isochratic. Separation quality: baseline separation. The first peak eluting off of the OD column provided (2S)-4-((3R*)-1′-(2-amino-5-chloro-4-pyrimidinyl)-1′,2′,4,5-tetrahydrospiro[furan-3,3′-indol]-6′-yl)-2-(1,3-thiazol-2-yl)-3-butyn-2-ol (199) as an off-white solid.

The second peak furnished (2R)-4-((3R*)-1′-(2-amino-5-chloro-4-pyrimidinyl)-1′,2′,4,5-tetrahydrospiro[furan-3,3′-indol]-6′-yl)-2-(1,3-thiazol-2-yl)-3-butyn-2-ol (200) as an off-white solid. Stereochemistry at the carbinol center assigned by analogy to defined structures based on biological activity.

### Example 201

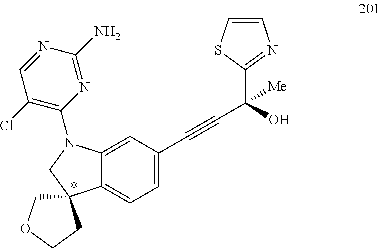

### (2S)-4-((3S*)-1′-(2-Amino-5-chloro-4-pyrimidinyl)-1′,2′,4,5-tetrahydrospiro[furan-3,3′-indol]-6′-yl)-2-(1,3-thiazol-2-yl)-3-butyn-2-ol

### Example 202

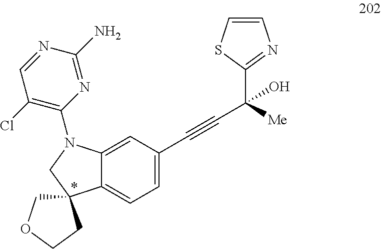

### (2R)-4-((3S*)-1′-(2-Amino-5-chloro-4-pyrimidinyl)-1′,2′,4,5-tetrahydrospiro[furan-3,3′-indol]-6′-yl)-2-(1,3-thiazol-2-yl)-3-butyn-2-ol

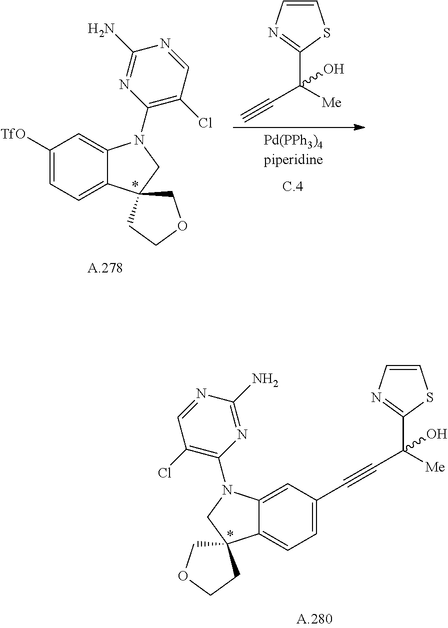

4-((3S*)-1′-(2-Amino-5-chloro-4-pyrimidinyl)-1′,2′,4,5-tetrahydrospiro[furan-3,3′-indol]-6′-yl)-2-(1,3-thiazol-2-yl)-3-butyn-2-ol A.280 was prepared from components A.278 and C.4 using chemistry similar to that described in Example 180. (an off-white solid)

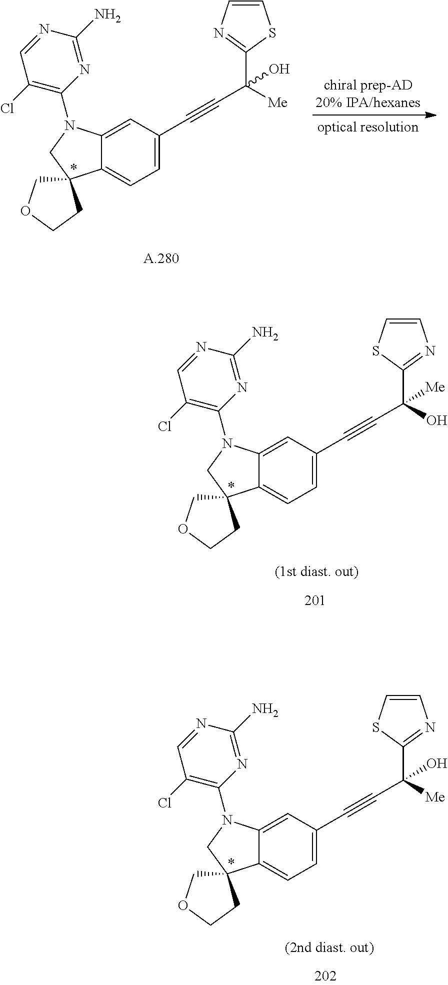

### (2S)-4-((3S*)-1′-(2-Amino-5-chloro-4-pyrimidinyl)-1′,2′,4,5-tetrahydrospiro[furan-3,3′-indol]-6′-yl)-2-(1,3-thiazol-2-yl)-3-butyn-2-ol (201) and (2R)-4-((3S*)-1′-(2-Amino-5-chloro-4-pyrimidinyl)-1′,2′,4,5-tetrahydrospiro[furan-3,3′-indol]-6′-yl)-2-(1,3-thiazol-2-yl)-3-butyn-2-ol (202)

Resolution of the diastereomeric mixture A.280 was accomplished by the following method. Instrument: Agilent 1100 series. Column: AD-preparative (50 mm×500 mm). Solvents: 20% isopropanol in hexanes. Gradient: isochratic. Separation quality: near baseline separation. The first peak eluting off of the AD column provided (2S)-4-((3S*)-1′-(2-amino-5-chloro-4-pyrimidinyl)-1′,2′,4,5-tetrahydrospiro[furan-3,3′-indol]-6′-yl)-2-(1,3-thiazol-2-yl)-3-butyn-2-ol (201) as an off-white solid.

The second peak furnished (2R)-4-((3S*)-1′-(2-amino-5-chloro-4-pyrimidinyl)-1′,2′,4,5-tetrahydrospiro[furan-3,3′-indol]-6′-yl)-2-(1,3-thiazol-2-yl)-3-butyn-2-ol (202) as an off-white solid. Stereochemistry at the carbinol center assigned by analogy to defined structures based on biological activity.

### Example 203

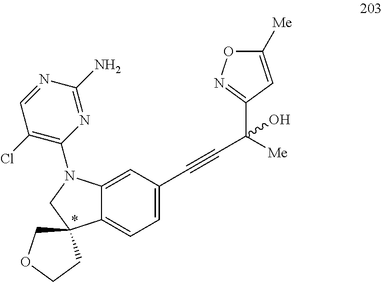

4-((3R*)-1′-(2-Amino-5-chloro-4-pyrimidinyl)-1′,2′,4,5-tetrahydrospiro[furan-3,3′-indol]-6′-yl)-2-(5-methyl-3-isoxazolyl)-3-butyn-2-ol (203) was prepared from components A.277 and C.1 using chemistry similar to that described in Example 180. (an off-white solid) ¹H NMR (400 MHz, DMSO-d₆) δ ppm 8.13 (1H, s), 7.28 (1H, d, J=7.8 Hz), 7.19 (1H, s), 7.01 (1 H, d, J=7.8 Hz), 6.70 (2H, s), 6.45 (1H, s), 6.33 (1H, s), 4.07-4.17 (2H, m), 3.93-4.02 (1 H, m), 3.89 (1H, q, J=7.8 Hz), 3.79 (1H, d, J=8.2 Hz), 3.65 (1H, dd, J=8.4, 1.4 Hz), 2.39 (3 H, s), 2.11-2.24 (2H, m), 1.77 (3H, s). LCMS-ESI (POS), M/Z, M+1: Found 452.0.

### Example 204

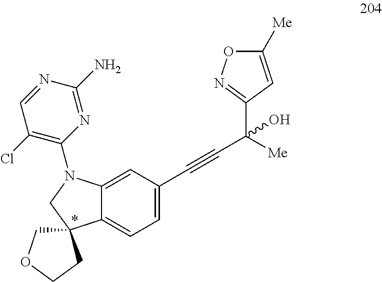

4-((3S*)-1′-(2-Amino-5-chloro-4-pyrimidinyl)-1′,2′,4,5-tetrahydrospiro[furan-3,3′-indol]-6′-yl)-2-(5-methyl-3-isoxazolyl)-3-butyn-2-ol (204) was prepared from components A.278 and C.1 using chemistry similar to that described in Example 180 as an off-white solid. ¹H NMR (400 MHz, DMSO-d₆) δ ppm 8.13 (1H, s), 7.28 (1H, d, J=7.8 Hz), 7.19 (1H, s), 7.01 (1 H, d, J=7.8 Hz), 6.70 (2H, s), 6.45 (1H, s), 6.33 (1H, s), 4.07-4.17 (2H, m), 3.93-4.02 (1 H, m), 3.89 (1H, q, J=7.8 Hz), 3.79 (1H, d, J=8.2 Hz), 3.65 (1H, dd, J=8.4, 1.4 Hz), 2.39 (3 H, s), 2.11-2.24 (2H, m), 1.77 (3H, s). LCMS-ESI (POS), M/Z, M+1: Found 452.0.

### Example 205

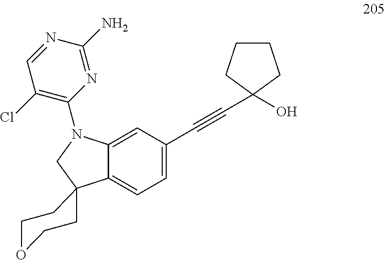

1-((1-(2-Amino-5-chloro-4-pyrimidinyl)-1,2,2′,3′,5′,6′-hexahydrospiro[indole-3,4′-pyran]-6-yl)ethynyl)cyclopentanol (205) was prepared from components A.101 and C.36 using chemistry similar to that described in Example 185 as an off-white solid in 70% yield. ¹H NMR (500 MHz, DMSO-d₆) δ ppm 8.13 (1H, s), 7.25 (1H, d, J=7.8 Hz), 7.06 (1H, d, J=1.0 Hz), 6.95 (1H, dd, J=7.8, 1.5 Hz), 6.70 (2H, s), 5.26 (1H, s), 4.12 (2H, s), 3.81-3.89 (2H, m), 3.47 (2H, t, J=11.5 Hz), 1.79-1.93 (6H, m), 1.61-1.78 (4H, m), 1.55 (2H, d, J=12.2 Hz). LCMS-ESI (POS), M/Z, M+1: Found 425.0.

### Example 206

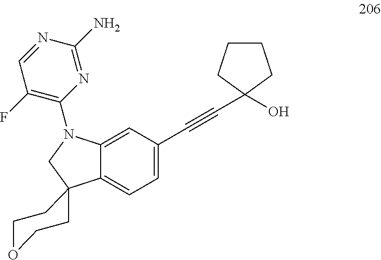

1-((1-(2-Amino-5-fluoro-4-pyrimidinyl)-1,2,2′,3′,5′,6′-hexahydrospiro[indole-3,4′-pyran]-6-yl)ethynyl)cyclopentanol (206) was prepared from components A.281 and C.36 using chemistry similar to that described in Example 185 as an off-white solid in 70% yield. ¹H NMR (500 MHz, DMSO-d₆) δ ppm 8.05 (1H, br. S.), 7.80 (1H, s), 7.26 (1H, d, J=7.8 Hz), 7.00 (1H, dd, J=7.8, 1.5 Hz), 6.37 (2H, s), 5.25 (1H, s), 4.17 (2H, d, J=3.9 Hz), 3.85 (2H, dd, J=11.7, 3.4 Hz), 3.46 (3H, t, J=11.2 Hz), 1.82-1.96 (6H, m), 1.62-1.77 (4H, m), 1.57 (2H, d, J=12.7 Hz). LCMS-ESI (POS), M/Z, M+1: Found 409.1.

### Example 207

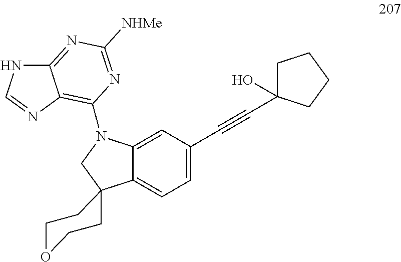

1-((1-(2-(Methylamino)-9H-purin-6-yl)-1,2,2′,3′,5′,6′-hexahydrospiro[indole-3,4′-pyran]-6-yl)ethynyl)cyclopentanol (207) was prepared from components A.283 and C.36 using chemistry similar to that described in Example 185 as an off-white solid. ¹H NMR (400 MHz, DMSO-d₆) δ ppm 12.43 (1H, br. s.), 8.69 (1H, br. s.), 7.86 (1H, s), 7.28 (1H, d, J=7.7 Hz), 7.01 (1H, dd, J=7.7, 1.5 Hz), 6.47-6.58 (1H, m), 5.26 (1H, s), 4.75 (2H, s), 3.92 (2H, dd, J=11.9, 3.5 Hz), 3.49 (2H, t, J=11.7 Hz), 2.88 (3H, d, J=4.8 Hz), 1.81-1.98 (6H, m), 1.64-1.80 (4H, m), 1.59 (2H, d, J=12.4 Hz). LCMS-ESI (POS), M/Z, M+1: Found 445.1.

### Example 208

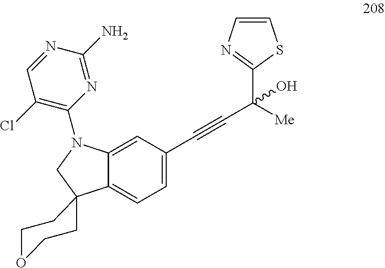

(rac)-4-(1-(2-Amino-5-chloro-4-pyrimidinyl)-1,2,2′,3′,5′,6′-hexahydrospiro[indole-3,4′-pyran]-6-yl)-2-(1,3-thiazol-2-yl)-3-butyn-2-ol (208) was prepared from components A.101 and C.4 using chemistry similar to that described in Example 185 as a light yellow solid. ¹H NMR (400 MHz, DMSO-d₆) δ ppm 8.14 (1H, s), 7.75 (1H, d, J=3.1 Hz), 7.66 (1H, d, J=3.5 Hz), 7.27 (1H, d, J=7.8 Hz), 7.07 (1H, s), 6.98 (1H, d, J=7.4 Hz), 6.94 (1H, s), 6.68 (2H, s), 4.12 (2H, s), 3.84 (2H, dd, J=11.3, 3.1 Hz), 3.47 (2H, t, J=11.9 Hz), 1.80-1.94 (5H, m), 1.55 (2H, d, J=13.3 Hz). LCMS-ESI (POS), M/Z, M+1: Found 468.1.

### Example 209

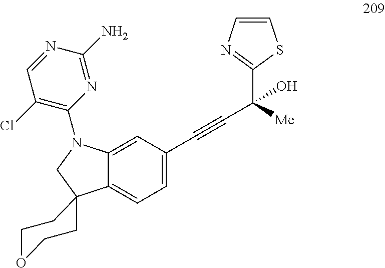

### (2R*)-4-(1-(2-amino-5-chloro-4-pyrimidinyl)-1,2,2′,3′,5′,6′-hexahydrospiro[indole-3,4′-pyran]-6-yl)-2-(1,3-thiazol-2-yl)-3-butyn-2-ol

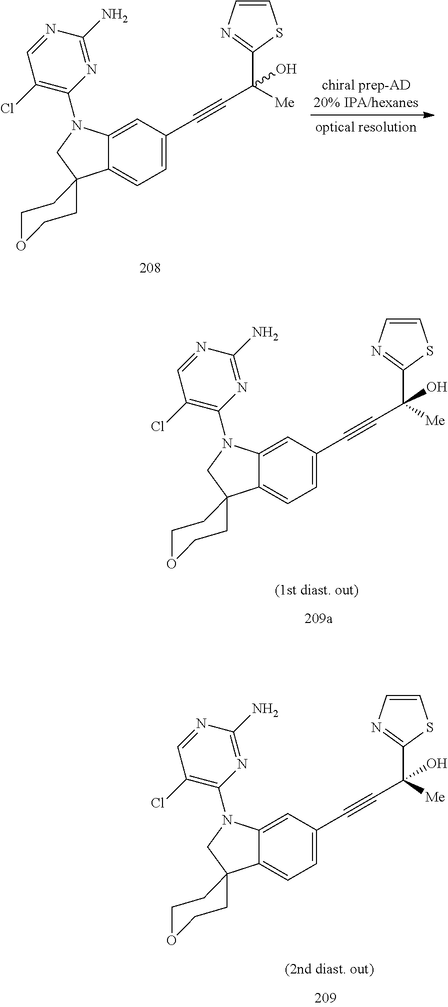

### (2S)-4-(1-(2-Amino-5-chloro-4-pyrimidinyl)-1,2,2′,3′,5′,6′-hexahydrospiro[indole-3,4′-pyran]-6-yl)-2-(1,3-thiazol-2-yl)-3-butyn-2-ol and (2R)-4-(1-(2-amino-5-chloro-4-pyrimidinyl)-1,2,2′,3′,5′,6′-hexahydrospiro[indole-3,4′-pyran]-6-yl)-2-(1,3-thiazol-2-yl)-3-butyn-2-ol (209)

Optical resolution of the racemic compound of example 208 was accomplished by the following method. Instrument: Agilent 1100 series. Column: AD-preparative (50 mm×500 mm). Solvents: 20% isopropanol in hexanes. Gradient: isochratic. Separation quality: near baseline separation. The first peak eluting off of the AD column provided (2S)-4-(1-(2-amino-5-chloro-4-pyrimidinyl)-1,2,2′,3′,5′,6′-hexahydrospiro[indole-3,4′-pyran]-6-yl)-2-(1,3-thiazol-2-yl)-3-butyn-2-ol (209a) as an off-white solid.

The second peak furnished (2R)-4-(1-(2-amino-5-chloro-4-pyrimidinyl)-1,2,2′,3′,5′,6′-hexahydrospiro[indole-3,4′-pyran]-6-yl)-2-(1,3-thiazol-2-yl)-3-butyn-2-ol (209) as an off-white solid. Stereochemistry assigned by analogy to defined structures based on biological activity.

### Example 210

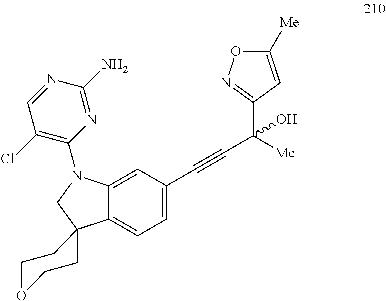

(rac)-4-(1-(2-Amino-5-chloro-4-pyrimidinyl)-1,2,2′,3′,5′,6′-hexahydrospiro[indole-3,4′-pyran]-6-yl)-2-(5-methyl-3-isoxazolyl)-3-butyn-2-ol (210) was prepared from components A.101 and C.1 using chemistry similar to that described in Example 185 as a light yellow solid.

¹H NMR (400 MHz, DMSO-d₆) δ ppm 8.14 (1H, s), 7.28 (1H, d, J=7.8 Hz), 7.10 (1H, d, J=1.2 Hz), 6.99 (1H, dd, J=7.6, 1.4 Hz), 6.69 (2H, s), 6.42 (1H, s), 6.32 (1H, s), 4.13 (2H, s), 3.84 (2H, dd, J=12.1, 2.7 Hz), 3.47 (2H, t, J=11.5 Hz), 2.39 (3H, s), 1.79-1.93 (2H, m), 1.76 (3H, s), 1.56 (2H, d, J=12.5 Hz). LCMS-ESI (POS), M/Z, M+1: Found 466.0.

### Example 211

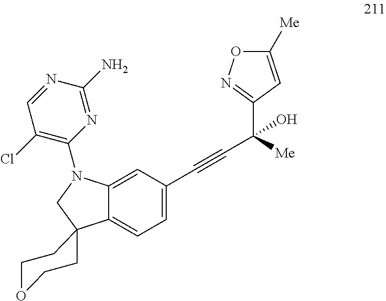

### (2R)-4-(1-(2-amino-5-chloro-4-pyrimidinyl)-1,2,2′,3′,5′,6′-hexahydrospiro[indole-3,4′-pyran]-6-yl)-2-(5-methyl-3-isoxazolyl)-3-butyn-2-ol

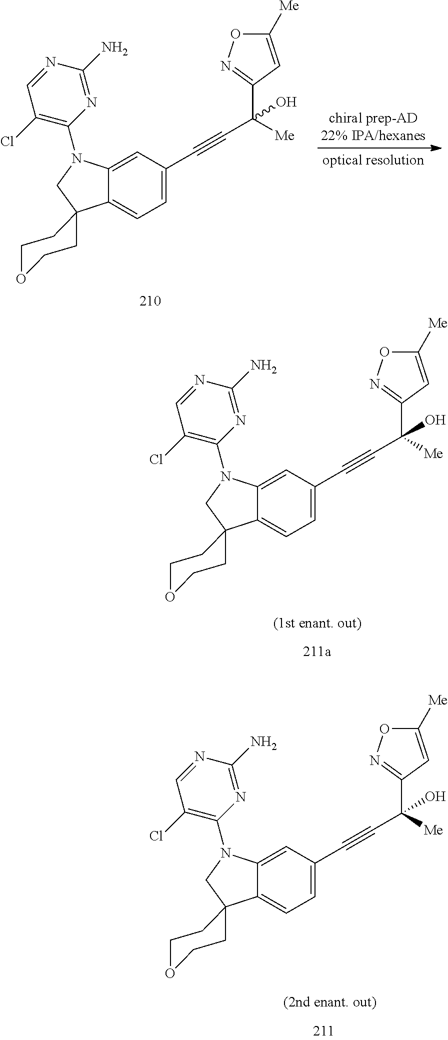

### (2S)-4-(1-(2-Amino-5-chloro-4-pyrimidinyl)-1,2,2′,3′,5′,6′-hexahydrospiro[indole-3,4′-pyran]-6-yl)-2-(5-methyl-3-isoxazolyl)-3-butyn-2-ol (211a) and (2R)-4-(1-(2-amino-5-chloro-4-pyrimidinyl)-1,2,2′,3′,5′,6′-hexahydrospiro[indole-3,4′-pyran]-6-yl)-2-(5-methyl-3-isoxazolyl)-3-butyn-2-ol (211)

Optical resolution of the racemic compound of example 210 was accomplished by the following method. Instrument: Agilent 1100 series. Column: AD-preparative (50 mm×500 mm). Solvents: 22% isopropanol in hexanes. Gradient: isochratic. Separation quality: no baseline separation. The first peak eluting off of the AD column provided (2S)-4-(1-(2-amino-5-chloro-4-pyrimidinyl)-1,2,2′,3′,5′,6′-hexahydrospiro[indole-3,4′-pyran]-6-yl)-2-(5-methyl-3-isoxazolyl)-3-butyn-2-ol (211a) in 98% ee as an off-white solid. The second peak furnished (2R)-4-(1-(2-amino-5-chloro-4-pyrimidinyl)-1,2,2′,3′,5′,6′-hexahydrospiro[indole-3,4′-pyran]-6-yl)-2-(5-methyl-3-isoxazolyl)-3-butyn-2-ol (211) (the more active enantiomer) in 98% ee as an off-white solid. Stereochemistry assigned by analogy to defined structures based on biological activity.

### Example 212

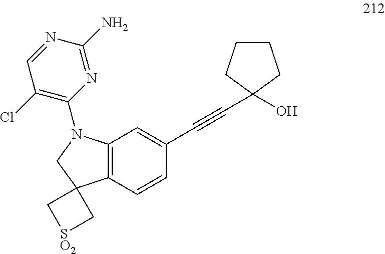

1-((1-(2-Amino-5-chloro-4-pyrimidinyl)-1′,1′-dioxido-1,2-dihydrospiro[indole-3,3′-thietan]-6-yl)ethynyl)cyclopentanol (212) was prepared as an off-white solid from components A.290 and C.36 using chemistry similar to that described in Example 185 except that piperidine was used as the reaction solvent. ¹H NMR (400 MHz, DMSO-d₆) δ ppm 8.15 (1 H, s), 7.62 (1H, d, J=7.8 Hz), 7.14 (1H, s), 7.05 (1H, dd, J=7.8, 1.2 Hz), 6.73 (2H, s), 5.29 (1 H, s), 4.61 (2H, d), 4.46-4.55 (4H, m), 1.79-1.96 (4H, m), 1.58-1.78 (4H, m). LCMS-ESI (POS), M/Z, M+1: Found 445.0.

### Example 213

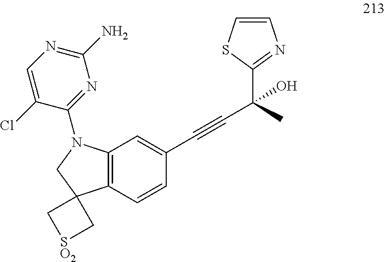

(2R)-4-(1-(2-Amino-5-chloro-4-pyrimidinyl)-1′,1′-dioxido-1,2-dihydrospiro[indole-3,3′-thietan]-6-yl)-2-(1,3-thiazol-2-yl)-3-butyn-2-ol (213) was prepared from components A.290 and C.6 using chemistry similar to that described in Example 185 as (an off-white solid). ¹H NMR (400 MHz, DMSO-d₆) δ ppm 8.16 (1H, s), 7.76 (1H, d, J=3.1 Hz), 7.59-7.70 (2H, m), 7.15 (1H, s), 7.08 (1H, dd, J=7.6, 1.4 Hz), 7.00 (1H, s), 6.73 (2H, s), 4.56-4.66 (2H, m), 4.43-4.56 (4H, m), 1.85 (3H, s). LCMS-ESI (POS), M/Z, M+1: Found 488.0.

### Example 214

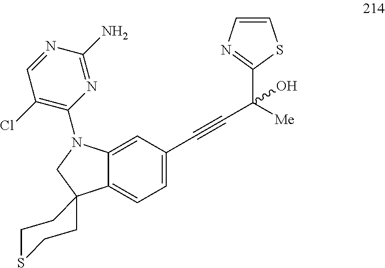

(rac)-4-(1-(2-Amino-5-chloro-4-pyrimidinyl)-1,2,2′,3′,5′,6′-hexahydrospiro[indole-3,4′-thiopyran]-6-yl)-2-(1,3-thiazol-2-yl)-3-butyn-2-ol (214) (an off-white solid) was prepared from components A.297 and C.4 using chemistry similar to that described in Example 185. ¹H NMR (400 MHz, CHLOROFORM-d) δ ppm 8.10 (1H, s), 7.77 (1H, d, J=3.1 Hz), 7.49 (1H, s), 7.35 (1H, d, J=3.1 Hz), 7.13 (2H, s), 4.99 (2H, br. s.), 4.10 (2H, s), 3.69 (1H, br. s.), 2.74-2.87 (2H, m), 2.56-2.68 (2H, m), 1.94-2.08 (7H, m). LCMS-ESI (POS), M/Z, M+1: Found 484.0.

### Example 215

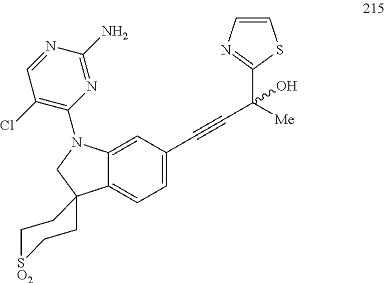

(rac)-4-(1-(2-Amino-5-chloro-4-pyrimidinyl)-1′,1′-dioxido-1,2,2′,3′,5′,6′-hexahydrospiro[indole-3,4′-thiopyran]-6-yl)-2-(1,3-thiazol-2-yl)-3-butyn-2-ol (215) (an off-white solid) was prepared from components A.301 and C.4 using chemistry similar to that described in Example 185. ¹H NMR (400 MHz, DMSO-d₆) δ ppm 8.16 (1H, s), 7.76 (1H, d, J=3.1 Hz), 7.67 (1H, d, J=3.1 Hz), 7.29 (1H, d, J=7.8 Hz), 6.93-7.05 (3H, m), 6.72 (2H, s), 4.19 (2H, s), 3.32-3.36 (2H, m), 3.08 (2H, d, J=10.6 Hz), 2.23-2.30 (2H, m), 2.05 (2H, d, J=12.5 Hz), 1.85 (3H, s). LCMS-ESI (POS), M/Z, M+1: Found 516.1.

### Example 216

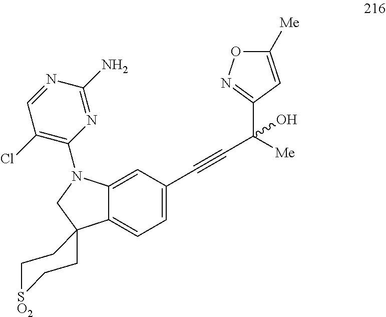

(rac)-4-(1-(2-Amino-5-chloro-4-pyrimidinyl)-1′,1′-dioxido-1,2,2′,3′,5′,6′-hexahydrospiro[indole-3,4′-thiopyran]-6-yl)-2-(5-methyl-3-isoxazolyl)-3-butyn-2-ol (216) (an off-white solid) was prepared from components A.301 and C.1 using chemistry similar to that described in Example 185. ¹H NMR (400 MHz, DMSO-d₆) δ ppm 8.16 (1H, s), 7.29 (1H, d, J=7.8 Hz), 6.93-7.06 (2H, m), 6.72 (2H, s), 6.44 (1H, s), 6.32 (1H, s), 4.19 (2H, s), 3.32-3.41 (2H, m), 3.08 (2H, d, J=13.3 Hz), 2.39 (3H, s), 2.22-2.35 (2H, m), 2.05 (2H, d, J=14.1 Hz), 1.76 (3H, s). LCMS-ESI (POS), M/Z, M+1: Found 514.2.

### Example 217

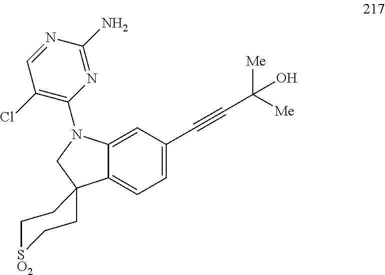

4-(1-(2-Amino-5-chloro-4-pyrimidinyl)-1′,1′-dioxido-1,2,2′,3′,5′,6′-hexahydrospiro[indole-3,4′-thiopyran]-6-yl)-2-methyl-3-butyn-2-ol (217) (an off-white solid) was prepared from components A.301 and C.35. using chemistry similar to that described in Example 185. ¹H NMR (400 MHz, DMSO-d₆) δ ppm 8.16 (1H, s), 7.26 (1H, d, J=7.8 Hz), 6.91-7.03 (2H, m), 6.72 (2H, s), 5.41 (1H, s), 4.18 (2H, s), 3.32-3.40 (2H, m), 3.08 (2H, d, J=13.3 Hz), 2.20-2.36 (2H, m), 2.05 (2H, d, J=14.1 Hz), 1.44 (6H, s). LCMS-ESI (POS), M/Z, M+1: Found 447.1.

### Example 218

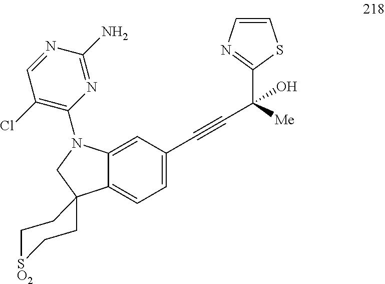

(2R)-4-(1-(2-Amino-5-chloro-4-pyrimidinyl)-1′,1′-dioxido-1,2,2′,3′,5′,6′-hexahydrospiro[indole-3,4′-thiopyran]-6-yl)-2-(1,3-thiazol-2-yl)-3-butyn-2-ol (218) (an off-white solid) was prepared from components A.301 and C.6 using chemistry similar to that described in Example 185. ¹H NMR (400 MHz, DMSO-d₆) δ ppm 8.16 (1H, s), 7.76 (1H, d, J=3.1 Hz), 7.67 (1H, d, J=3.1 Hz), 7.29 (1H, d, J=7.8 Hz), 6.93-7.05 (3H, m), 6.72 (2H, s), 4.19 (2H, s), 3.32-3.36 (2H, m), 3.08 (2H, d, J=10.6 Hz), 2.23-2.30 (2H, m), 2.05 (2H, d, J=12.5 Hz), 1.85 (3H, s). LCMS-ESI (POS), M/Z, M+1: Found 516.1.

### Example 219

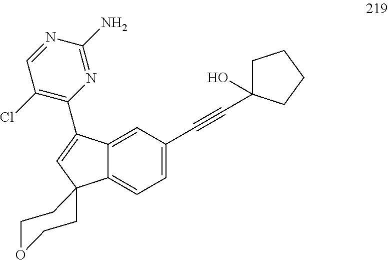

1-((3-(2-Amino-5-chloro-4-pyrimidinyl)-2′,3′,5′,6′-tetrahydrospiro[indene-1,4′-pyran]-5-yl)ethynyl)cyclopentanol (219) was prepared from components A.310 and C.36 using chemistry similar to that described in Example 185. (61% yield as an off-white solid) ¹H NMR (400 MHz, CHLOROFORM-d) δ ppm 8.38 (1H, s), 7.67 (1H, s), 7.55 (1H, s), 7.37 (2H, s), 5.17 (2H, br. s.), 4.13 (2H, ddd, J=12.1, 4.4, 2.2 Hz), 3.82 (2H, td, J=12.1, 1.8 Hz), 2.22-2.33 (2H, m), 1.99-2.13 (4H, m), 1.74-1.92 (4H, m), 1.44 (2H, d, J=13.2 Hz). LCMS-ESI (POS), M/Z, M+1: Found 422.2.

### Example 220

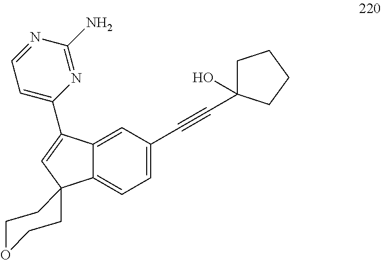

1-((3-(2-Amino-4-pyrimidinyl)-2′,3′,5′,6′-tetrahydrospiro[indene-1,4′-pyran]-5-yl)ethynyl)cyclopentanol (220) (an off-white solid) was prepared from components A.309 and C.36 using chemistry similar to that described in Example 185. ¹H NMR (400 MHz, CHLOROFORM-d) δ ppm 8.38 (1H, d, J=4.8 Hz), 8.10 (1H, s), 7.53 (1H, s), 7.34-7.43 (2 H, m), 7.00 (1H, d, J=5.1 Hz), 5.16 (2H, br. s.), 4.13 (2H, d, J=12.4 Hz), 3.83 (2H, t, J=11.9 Hz), 2.25 (2H, td, J=12.6, 4.0 Hz), 1.99-2.16 (4H, m), 1.74-1.96 (4H, m), 1.39 (1H, d, J=13.2 Hz). LCMS-ESI (POS), M/Z, M+1: Found 388.1.

### Example 221

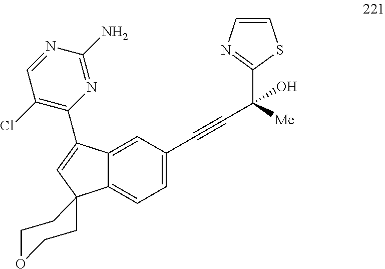

(2R)-4-(3-(2-Amino-5-chloro-4-pyrimidinyl)-2′,3′,5′,6′-tetrahydrospiro[indene-1,4′-pyran]-5-yl)-2-(1,3-thiazol-2-yl)-3-butyn-2-ol (221) (an off-white solid) was prepared from components A.310 and C.6 using chemistry similar to that described in Example 185. LCMS-ESI (POS), M/Z, M+1: Found 465.1.

### Example 222

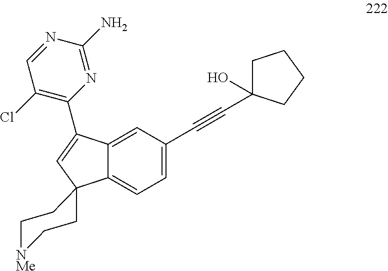

1-((3-(2-Amino-5-chloro-4-pyrimidinyl)-1′-methylspiro[indene-1,4′-piperidin]-5-yl)ethynyl)cyclopentanol (222) was prepared from components A.327 and C.36 using chemistry similar to that described in Example 185 as (an off-white solid). ¹H NMR (400 MHz, CHLOROFORM-d) δ ppm 8.35 (1H, s), 7.63 (1H, s), 7.43 (1H, s), 7.29-7.37 (2H, m), 5.31 (2H, s), 3.04 (2H, d, J=11.0 Hz), 2.27-2.50 (7H, m), 1.97-2.11 (4H, m), 1.70-1.93 (4H, m), 1.50 (2H, d, J=12.5 Hz). LCMS-ESI (POS), M/Z, M+1: Found 435.2.

### Example 223

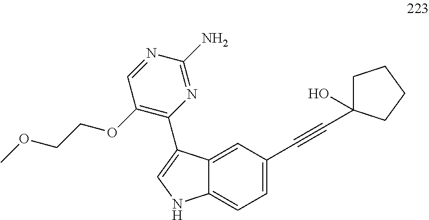

1-((3-(2-Amino-5-(2-methoxyethoxy)-4-pyrimidinyl)-1H-indol-5-yl)ethynyl)cyclopentanol

(223) was prepared from components A.313 and C.36 using chemistry similar to that described in Example 185. ¹H NMR (400 MHz, DMSO-d₆) δ ppm 11.80 (1H, d, J=2.2 Hz), 8.78 (1H, s), 8.43 (1H, d, J=2.9 Hz), 7.41 (1H, d, J=8.4 Hz), 7.19 (1H, dd, J=8.2, 1.6 Hz), 6.09 (2H, br. s.), 5.20 (1H, s), 4.12-4.21 (2H, m), 3.70-3.77 (2H, m), 3.36 (3H, s), 1.94 (4

H, t, J=5.3 Hz), 1.63-1.82 (4H, m). LCMS-ESI (POS), M/Z, M+1: Found 393.2.

### Example 224

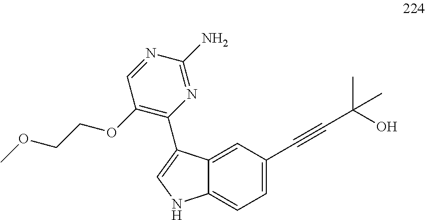

4-(3-(2-Amino-5-(2-methoxyethoxy)-4-pyrimidinyl)-1H-indol-5-yl)-2-methyl-3-butyn-2-ol

(224) was prepared from components A.313 and C.35 using chemistry similar to that described in Example 185. ¹H NMR (400 MHz, DMSO-d₆) δ ppm 11.82 (1H, br. s.), 8.80 (1H, s), 8.45 (1H, d, J=2.6 Hz), 7.43 (1H, d, J=8.4 Hz), 7.20 (1H, dd, J=8.2, 1.6 Hz), 6.12 (2H, br. s.), 5.36 (1H, s), 4.13-4.22 (2H, m), 3.71-3.78 (2H, m), 3.37 (3H, s), 1.53 (6H, s). LCMS-ESI (POS), M/Z, M+1: Found 367.3.

### Example 225

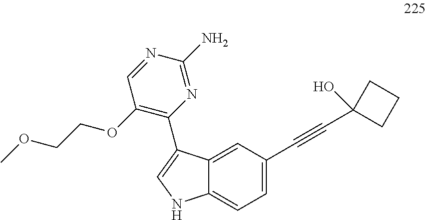

1-((3-(2-Amino-5-(2-methoxyethoxy)-4-pyrimidinyl)-1H-indol-5-yl)ethynyl)cyclobutanol

(225) was prepared from compounds A.313 and C.28 using chemistry similar to that described in Example 185. ¹H NMR (400 MHz, DMSO-d₆) δ ppm 11.81 (1H, d, J=1.8 Hz), 8.82 (1H, s), 8.43 (1H, d, J=2.9 Hz), 8.06 (1H, s), 7.42 (1H, d, J=7.7 Hz), 7.21 (1H, dd, J=8.4, 1.5 Hz), 6.10 (2H, s), 5.76 (1H, s), 4.13-4.21 (2H, m), 3.70-3.76 (2H, m), 3.36 (3H, s), 2.44 (2H, ddd, J=4.9, 2.4, 2.2 Hz), 2.17-2.28 (2H, m), 1.76-1.86 (2H, m). LCMS-ESI (POS), M/Z, M+1: Found 379.1.

### Example 226

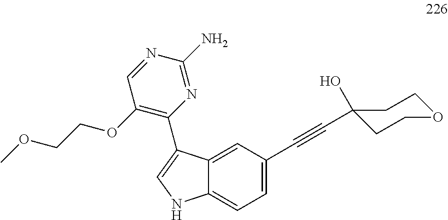

4-((3-(2-Amino-5-(2-methoxyethoxy)-4-pyrimidinyl)-1H-indol-5-yl)ethynyl)tetrahydro-2H-pyran-4-ol (226) was prepared from components A.313 and C.40 using chemistry similar to that described in Example 185. ¹H NMR (400 MHz, CHLOROFORM-d) δ ppm 11.82 (1H, d, J=2.2 Hz), 8.81 (1H, d, J=1.5 Hz), 8.43 (1H, d, J=2.6 Hz), 8.06 (1H, s), 7.43 (1H, dd, J=8.4, 0.7 Hz), 7.22 (1H, dd, J=8.4, 1.5 Hz), 6.08 (2H, s), 5.61 (1H, s), 4.12-4.20 (2H, m), 3.76-3.85 (2H, m), 3.70-3.76 (2H, m), 3.63 (2H, ddd, J=11.3, 8.4, 2.9 Hz), 3.36 (3H, s), 1.88-1.98 (2H, m), 1.73 (2H, ddd, J=12.6, 8.8, 3.5 Hz). LCMS-ESI (POS), M/Z, M+1:

Found 409.1.

### Example 227

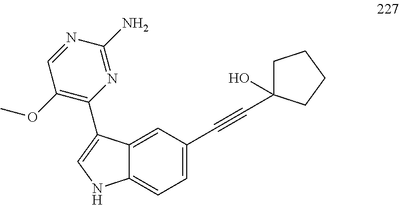

1-((3-(2-Amino-5-methoxy-4-pyrimidinyl)-1H-indol-5-yl)ethynyl)cyclopentanol (227) was prepared from components A.329 and C.36 using chemistry similar to that described in Example 185. ¹H NMR (500 MHz, DMSO-d₆) δ ppm 11.77 (1H, br. s.), 8.78 (1H, s), 8.29 (1 H, d, J=2.9 Hz), 7.42 (1H, d, J=8.3 Hz), 7.19 (1H, dd, J=8.6, 1.7 Hz), 6.25 (2H, s), 5.21 (1H, s), 3.88 (3H, s), 1.94 (4H, t, J=4.9 Hz), 1.65-1.83 (4H, m). LCMS-ESI (POS), M/Z, M+1:

Found 349.1.

### Example 228

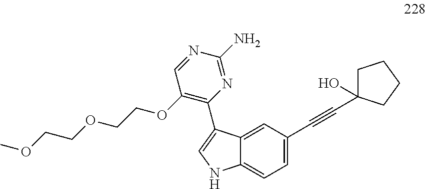

1-((3-(2-Amino-5-(2-(2-methoxyethoxy)ethoxy)-4-pyrimidinyl)-1H-indol-5-yl)ethynyl)cyclopentanol (228) was prepared from components A.330 and C.36 using chemistry similar to that described in Example 185. ¹H NMR (400 MHz, DMSO-d₆) δ ppm 11.76 (1H, s), 8.78 (1H, d, J=0.7 Hz), 8.45 (1H, s), 8.05 (1H, s), 7.42 (1H, d, J=8.4 Hz), 7.18 (1H, dd, J=8.4, 1.8 Hz), 6.08 (2H, s), 5.21 (1H, s), 4.12-4.19 (2H, m), 3.78-3.84 (2

H, m), 3.59-3.65 (2H, m), 3.48-3.53 (2H, m), 3.26 (3H, s), 1.89-1.97 (4H, m), 1.64-1.80 (4H, m). LCMS-ESI (POS), M/Z, M+1: Found 437.1.

### Example 229

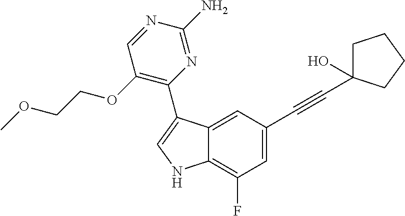

1-((3-(2-Amino-5-(2-methoxyethoxy)-4-pyrimidinyl)-7-fluoro-1H-indol-5-yl)ethynyl)cyclopentanol (229) was prepared from components A.328 and C.36 using chemistry similar to that described in Example 185. ¹H NMR (400 MHz, DMSO-d₆) δ ppm 12.33 (1H, br. s.), 8.62 (1H, s), 8.46 (1H, d, J=1.8 Hz), 8.10 (1H, br. s.), 7.02 (1H, d, J=11.7 Hz), 6.15 (2H, s), 5.24 (1H, s), 4.12-4.23 (2H, m), 3.67-3.77 (2H, m), 3.36 (3H, s), 1.87-2.03 (4H, m), 1.61-1.82 (4H, m). LCMS-ESI (POS), M/Z, M+1: Found 411.2.

### Example 230

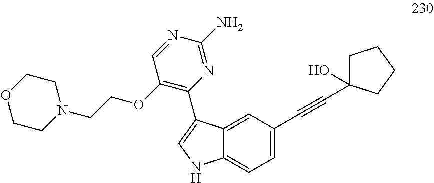

1-((3-(2-Amino-5-(2-(4-morpholinyl)ethoxy)-4-pyrimidinyl)-1H-indol-5-yl)ethynyl)cyclopentanol (230) was prepared from components A.331 and C.36 using chemistry similar to that described in Example 185. LCMS-ESI (POS), M/Z, M+1: Found 448.1.

### Example 231

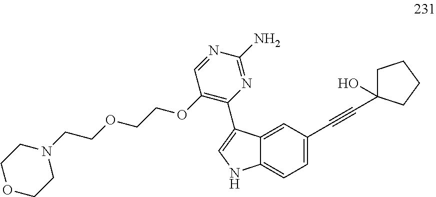

1-((3-(2-Amino-5-(2-(2-(4-morpholinyl)ethoxy)ethoxy)-4-pyrimidinyl)-1H-indol-5-yl)ethynyl)cyclopentanol (231) was prepared from components A.332 and C.36 using chemistry similar to that described in Example 185. LCMS-ESI (POS), M/Z, M+1: Found 492.3.

### Example 232

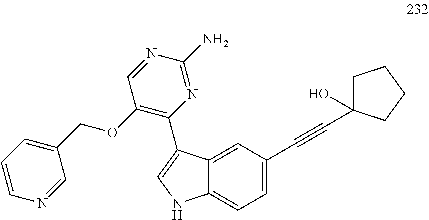

1-((3-(2-Amino-5-(3-pyridinylmethoxy)-4-pyrimidinyl)-1H-indol-5-yl)ethynyl)cyclopentanol (232) was prepared from components A.333 and C.36 using chemistry similar to that described in Example 185. ¹H NMR (400 MHz, DMSO-d₆) δ ppm 11.74 (1H, br. s.), 8.66-8.79 (2H, m), 8.58 (1H, br. s.), 8.19 (1H, d, J=1.5 Hz), 8.14 (1H, s), 7.92 (1H, d, J=8.4 Hz), 7.34-7.50 (2H, m), 7.18 (1H, d, J=8.4 Hz), 6.14 (2H, s), 5.21 (3 H, s), 1.88-2.00 (4H, m), 1.64-1.81 (4H, m). LCMS-ESI (POS), M/Z, M+1: Found 426.2.

### Example 233

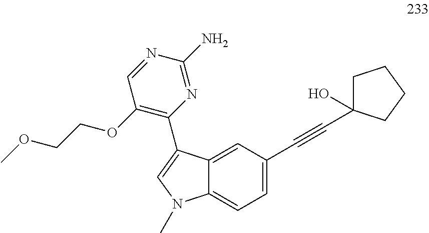

1-((3-(2-Amino-5-(2-methoxyethoxy)-4-pyrimidinyl)-1-methyl-1H-indol-5-yl)ethynyl)cyclopentanol (233) was prepared from components A.317 and C.36 using chemistry similar to that described in Example 185. ¹H NMR (400 MHz, DMSO-d₆) δ ppm 8.80 (1H, d, J=1.5 Hz), 8.42 (1H, s), 8.07 (1H, br. s.), 7.49 (1H, d, J=8.4 Hz), 7.26 (1H, dd, J=8.4, 1.8 Hz), 6.12 (2H, s), 5.22 (1H, s), 4.12-4.19 (2H, m), 3.86 (3H, s), 3.74 (2H, dd, J=5.5, 3.7 Hz), 3.39 (3H, s), 1.87-1.98 (4H, m), 1.65-1.81 (4H, m). LCMS-ESI (POS),

M/Z, M+1: Found 407.5.

### Example 234

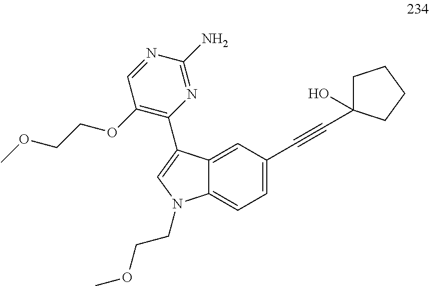

1-((3-(2-Amino-5-(2-methoxyethoxy)-4-pyrimidinyl)-1-(2-methoxyethyl)-1H-indol-5-yl)ethynyl)cyclopentanol (234) was prepared from components A.319 and C.36 using chemistry similar to that described in Example 185. LCMS-ESI (POS), M/Z, M+1: Found 451.1.

### Example 235

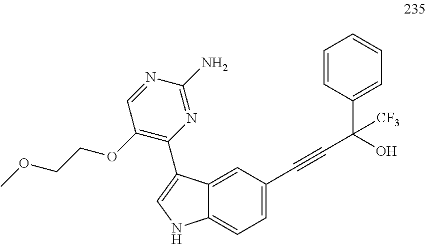

4-(3-(2-Amino-5-(2-methoxyethoxy)-4-pyrimidinyl)-1H-indol-5-yl)-1,1,1-trifluoro-2-phenyl-3-butyn-2-ol (235) was prepared from components A.313 and C.51 using chemistry similar to that described in Example 185. ¹H NMR (400 MHz, DMSO-d₆) δ ppm 11.92 (1H, s), 8.93 (1H, s), 8.46 (1H, d, J=2.6 Hz), 8.08 (1H, s), 7.78-7.87 (3H, m), 7.42-7.54 (4H, m), 7.35 (1H, dd, J=8.4, 1.8 Hz), 6.07 (2H, s), 4.14-4.22 (2H, m), 3.70-3.76 (2H, m), 3.36 (3H, s). LCMS-ESI (POS), M/Z, M+1: Found 483.4.

### Example 236

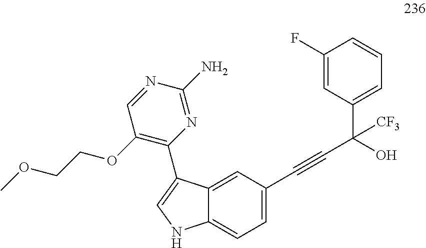

4-(3-(2-Amino-5-(2-methoxyethoxy)-4-pyrimidinyl)-1H-indol-5-yl)-1,1,1-trifluoro-2-(3-fluorophenyl)-3-butyn-2-ol (236) was prepared from components A.313 and C.52 using chemistry similar to that described in Example 185. ¹H NMR (400 MHz, DMSO-d₆) δ ppm 11.94 (1H, d, J=2.2 Hz), 8.95 (1H, s), 8.46 (1H, d, J=2.6 Hz), 8.08 (1H, s), 8.02 (1H, s), 7.69 (1H, d, J=8.1 Hz), 7.48-7.60 (3H, m), 7.27-7.39 (2H, m), 6.08 (2H, s), 4.13-4.21 (2 H, m), 3.69-3.78 (2H, m), 3.36 (3H, s). LCMS-ESI (POS), M/Z, M+1: Found 501.4.

### Example 237

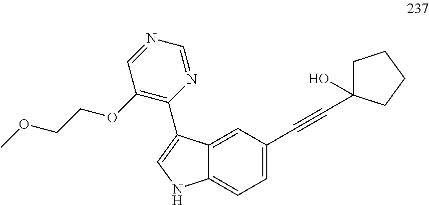

1-((3-(5-(2-Methoxyethoxy)-4-pyrimidinyl)-1H-indol-5-yl)ethynyl)cyclopentanol (237) was prepared from components A.315 and C.36 using chemistry similar to that described in Example 185. ¹H NMR (400 MHz, DMSO-d₆) δ ppm 11.96 (1H, br. s.), 8.85 (2H, s), 8.43-8.58 (2H, m), 7.46 (1H, d, J=8.4 Hz), 7.22 (1H, dd, J=8.2, 1.6 Hz), 5.75 (1H, s), 5.27 (1H, s), 4.36-4.48 (2H, m), 3.80-3.87 (2H, m), 3.39 (3H, s), 1.87-1.97 (4H, m), 1.62-1.81 (4 H, m). LCMS-ESI (POS), M/Z, M+1: Found 378.4.

### Example 238

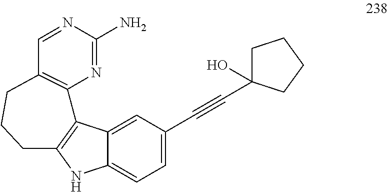

### 1-((2-Amino-5,6,7,8-tetrahydropyrimido[4′,′:3,4]cyclohepta[1,2-b]indol-11-yl)ethynyl)cyclopentanol (238)

was prepared from components A.324 and C.36 using chemistry similar to that described in Example 185. ¹H NMR (400 MHz, DMSO-d₆) δ ppm 11.59 (1H, s), 8.72 (1H, d, J=1.5 Hz), 7.92 (1H, br. s.), 7.26 (1H, d, J=8.4 Hz), 7.12 (1H, dd, J=8.2, 1.6 Hz), 6.19 (2H, s), 5.18 (1H, s), 3.17 (2H, t, J=6.4 Hz), 2.59-2.65 (2H, m), 1.85-1.98 (6H, m), 1.62-1.82 (4H, m). LCMS-ESI (POS), M/Z, M+1: Found 359.2.

### Example 239

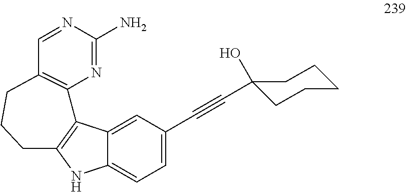

1-((2-Amino-5,6,7,8-tetrahydropyrimido[4′,′:3,4]cyclohepta[1,2-b]indol-11-yl)ethynyl)cyclohexanol (239) was prepared from components A.324 and C.37 using chemistry similar to that described in Example 185. ¹H NMR (400 MHz, DMSO-d₆) δ ppm 11.59 (1H, s), 8.71 (1H, s), 7.27 (1H, d, J=8.1 Hz), 7.13 (1H, dd, J=8.2, 1.6 Hz), 6.17 (2H, br. s.), 5.27 (1H, s), 3.17 (2H, t, J=6.6 Hz), 2.60-2.66 (2H, m), 1.81-2.04 (4H, m), 1.46-1.75 (8H, m). LCMS-ESI (POS), M/Z, M+1: Found 373.3.

### Example 240

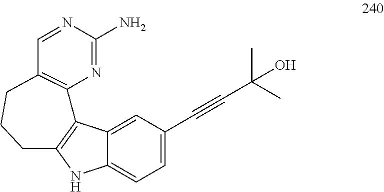

4-(2-Amino-5,6,7,8-tetrahydropyrimido[4′,5′:3,4]cyclohepta[1,2-b]indol-11-yl)-2-methyl-3-butyn-2-ol (240) was prepared from components A.324 and C.35 using chemistry similar to that described in Example 185. ¹H NMR (400 MHz, DMSO-d₆) δ ppm 11.59 (1H, s), 8.71 (1 H, s), 7.92 (1H, s), 7.26 (1H, d, J=8.4 Hz), 7.11 (1H, d, J=8.4 Hz), 6.19 (2H, s), 5.32 (1H, s), 3.17 (2H, t, J=6.6 Hz), 2.59-2.66 (2H, m), 1.86-2.02 (2H, m), 1.50 (6H, s). LCMS-ESI (POS), M/Z, M+1: Found 333.1.

### Example 241

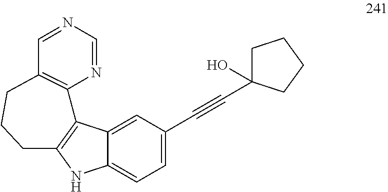

1-(5,6,7,8-Tetrahydropyrimido[4′,′:3,4]cyclohepta[1,2-b]indol-11-ylethynyl)cyclopentanol (241) was prepared from components A.325 and C.36 using chemistry similar to that described in Example 185. LCMS-ESI (POS), M/Z, M+1: Found 344.1.

### Example 242

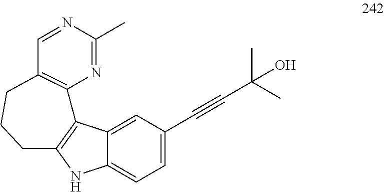

2-Methyl-4-(2-methyl-5,6,7,8-tetrahydropyrimido[4′,′:3,4]cyclohepta[1,2-b]indol-1′-yl)-3-butyn-2-ol (242) was prepared from components A.326 and C.35 using chemistry similar to that described in Example 185. ¹H NMR (400 MHz, DMSO-d₆) δ ppm 12.04 (1H, s), 8.76 (1 H, s), 8.43 (1H, s), 7.35 (1H, d, J=8.4 Hz), 7.20 (1H, d, J=8.4 Hz), 2.81-2.94 (2H, m), 2.72 (3H, s), 2.40-2.44 (2H, m), 1.94-2.10 (2H, m), 1.50 (6H, s). LCMS-ESI (POS), M/Z, M+1: Found 332.2.

### Example 243

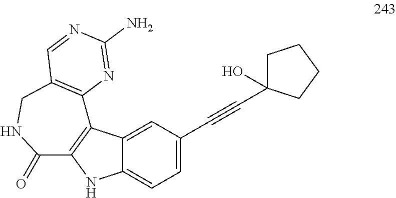

### 2-Amino-11-((1-hydroxycyclopentyl)ethynyl)-5,8-dihydropyrimido[4′,′:5,6]azepino[3,4-b]indol-7(6H)-one (243)

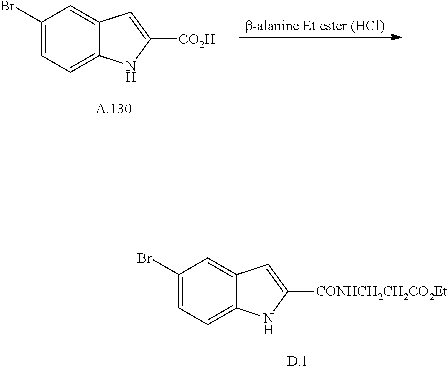

### Ethyl N-((5-bromo-1H-indol-2-yl)carbonyl)-beta-alaninate D.1

Ref. Chacun-Lefèvre, L., B. Joseph, et al. (2000). Tetrahedron 56(26): 4491-4499.

To a stirred ice-cooled solution of β-analine ethyl ester (HCl salt) (1.1 eq., 7.2 g) and DMAP (1.6 eq., 8.2 g) in dichloromethane (200 mL) was portion-wise added 5-bromo-1H-indole-2-carboxylic acid A.130 (10 g, 41.7 mmol) over a period of 10 min. The resulting mixture was stirred at 0° C. for 10 min before EDCI (1.1 eq., 8.8 g) was added in 3 portions over a period of 10 min. The resulting mixture was stirred at 0° C. and gradually warmed up to rt and stirred at ambient temperature for 19 h. Upon workup, the volatiles were removed in vacuo and the residue was diluted with water and extracted with ethyl acetate (2×). The combined organics were sequentially washed with water (2×), 2 N HCl aqueous solution (2×), and brine (2×), and dried over Na₂SO₄. The residue was triturated with ethyl acetate/hexanes to give pure ethyl N-((5-bromo-1H-indol-2-yl)carbonyl)-beta-alaninate D.1 (13.3 g, 94% yield) as an off-white solid. ¹H NMR (400 MHz, DMSO-d₆) δ ppm 11.77 (1H, s), 8.62 (1H, t, J=5.7 Hz), 7.84 (1H, d, J=1.8 Hz), 7.33-7.43 (1H, m), 7.23-7.33 (1H, m), 7.07 (1H, s), 4.07 (2H, q, J=7.0 Hz), 3.40-3.59 (2H, m), 2.59 (2H, t, J=7.0 Hz), 1.18 (3H, t, J=7.1 Hz). LCMS-ESI (POS), M/Z, M+1: Found 339.0 and 341.0.

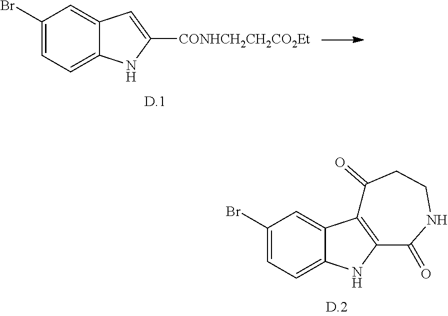

### 7-Bromo-3,4-dihydroazepino[3,4-b]indole-1,5(2H,10H)-dione D.2

Step 1. A mixture of ethyl N-((5-bromo-1H-indol-2-yl)carbonyl)-beta-alaninate D.1 (8.0 g, 23.6 mmol) and lithium hydroxide monohydrate (5 eq., 5.0 g) in a mixed solvent consisting of methanol (100 mL) and water (30 mL) was heated at reflux for 2 h. After cooling, the mixture was poured into ice and 2N HCl aqueous solution and extracted with ethyl acetate (2×). The combined organics were washed with brine (2×), dried over Na₂SO₄, and concentrated in vacuo. The residue was triturated with ethyl acetate/hexanes to give the intermediate, N-((5-bromo-1H-indol-2-yl)carbonyl)-beta-alanine (7.2 g, ˜quant. yield) as an off-white solid. ¹H NMR (400 MHz, DMSO-d₆) δ ppm 12.24 (1H, br. s.), 11.77 (1H, br. s.), 8.61 (1H, t, J=5.1 Hz), 7.83 (1H, s), 7.38 (1H, d, J=8.4 Hz), 7.24-7.32 (1H, m), 7.08 (1H, s), 3.48 (2H, q, J=6.7 Hz), 2.50-2.60 (2H, m). LCMS-ESI (POS), M/Z, M+1: Found 331.2 and 313.2.

Step 2. To a preheated mixture of polyphosphoric acid (16.0 g) and P₂O₅ (2.5 g) at 110° C. was added N-((5-bromo-1H-indol-2-yl)carbonyl)-beta-alanine (4.0 g, 12.8 mmol). The resulting mixture was stirred at 120° C. for 4.5 h. Upon workup, the mixture was cooled and quenched with ice water and extracted with ethyl acetate (3×). The combined organics were washed with brine (3×), dried over Na₂SO₄, and concentrated in vacuo. The residue was triturated with ethyl acetate/hexanes to give 7-bromo-3,4-dihydroazepino[3,4-b]indole-1,5(2H,10H)-dione D.2 (2.5 g, 70% yield) as an off-white solid. ¹H NMR (400 MHz, DMSO-d₆) δ ppm 12.66 (1H, br. s.), 8.81 (1H, t, J=4.6 Hz), 8.44 (1H, s), 7.41-7.63 (2H, m), 3.41-3.59 (2H, m), 2.77-2.96 (2H, m). LCMS-ESI (POS), M/Z, M+1: Found 293.2 and 295.2.

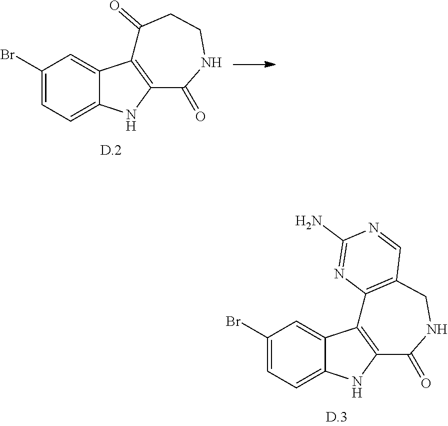

### 2-Amino-11-bromo-5,8-dihydropyrimido[4′,′:5,6]azepino[3,4-b]indol-7(6H)-one

7-Bromo-3,4-dihydroazepino[3,4-b]indole-1,5(2H,10H)-dione D.2 (0.47 g, 1.6 mmol) was treated neat with t-BuOCH(NMe₂)₂ (2.0 eq., 0.56 g) at 105° C. under N₂ for 40 min. The mixture was cooled before n-propanol (10 mL), guanidine hydrochloride (5.0 eq., 0.77 g), and sodium methoxide (5.53 M in methanol) (3.0 eq., 0.9 mL) were added sequentially. The resulting mixture was stirred at 95° C. for 30 min. Upon workup, the mixture was diluted with ice water and the precipitated was collected by vacuum filtration (repeated twice) to give 2-amino-1′-bromo-5,8-dihydropyrimido[4′,′:5,6]azepino[3,4-b]indol-7(6H)-one D.3 (0.47 g, 85% yield) as a yellow solid. ¹H NMR (400 MHz, DMSO-d₆) δ ppm 8.70 (1H, s), 8.48 (1H, br. s.), 8.23 (1H, s), 7.33-7.52 (2H, m), 6.67 (2H, br. s.), 3.96 (2H, s). LCMS-ESI (POS), M/Z, M+ 1: Found 344.2 and 346.3.

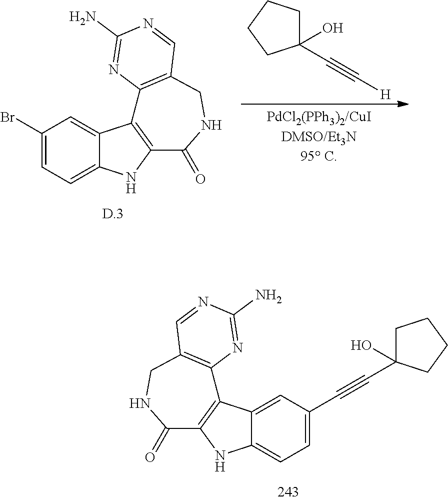

2-Amino-1′-((1-hydroxycyclopentyl)ethynyl)-5,8-dihydropyrimido[4′,′:5,6]azepino[3,4-b]indol-7(6H)-one (243) was prepared from 2-amino-1′-bromo-5,8-dihydropyrimido[4′,′:5,6]azepino[3,4-b]indol-7(6H)-one (D.3) and C.36 using chemistry similar to that described in Example 185. ¹H NMR (400 MHz, DMSO-d₆) δ ppm 12.41 (1H, s), 8.56 (1H, s), 8.49 (1H, t, J=5.1 Hz), 8.23 (1H, s), 7.49 (1H, d, J=8.4 Hz), 7.33 (1H, dd, J=8.6, 1.6 Hz), 6.67 (2H, s), 5.25 (1H, s), 3.96 (2H, d, J=4.8 Hz), 1.88-1.97 (4H, m), 1.65-1.81 (4H, m). LCMS-ESI (POS), M/Z, M+1: Found 374.4.

### Example 244

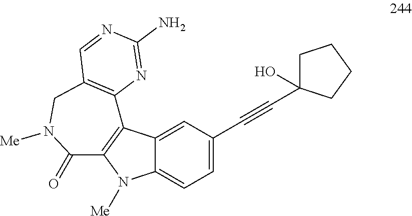

### 2-Amino-1′-((1-hydroxycyclopentypethynyl)-6,8-dimethyl-5,8-dihydropyrimido[4′,′:5,6]azepino[3,4-b]indol-7(6H)-one

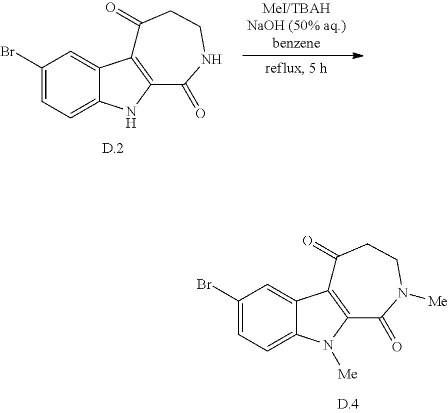

7-Bromo-2,10-dimethyl-3,4-dihydroazepino[3,4-b]indole-1,5(2H,10H)-dione (D.4) was prepared from 7-bromo-3,4-dihydroazepino[3,4-b]indole-1,5(2H,10H)-dione D.2 via bis-alkylation with iodomethane under phase transfer catalysis in benzene/50% aqueous sodium hydroxide at reflux for 5 hr.

¹H NMR (500 MHz, DMSO-d₆) δ ppm 8.35 (1H, d, J=1.0 Hz), 7.67 (1H, d, J=8.8 Hz), 7.54 (1H, ddd, J=8.8, 2.0, 1.0 Hz), 3.98 (3H, s), 3.76 (2H, br. s.), 3.17 (3H, s), 2.81-2.93 (2H, m). LCMS-ESI (POS), M/Z, M+ 1: Found 321.2 and 323.2.

2-Amino-1′-((1-hydroxycyclopentyl)ethynyl)-6,8-dimethyl-5,8-dihydropyrimido[4′,5′:5,6]azepino[3,4-b]indol-7(6H)-one (244) was prepared from 7-bromo-2,10-dimethyl-3,4-dihydroazepino[3,4-b]indole-1,5(2H,10H)-dione D.4 using chemistry similar to that described in Example 243. ¹H NMR (400 MHz, DMSO-d₆) δ ppm 8.51 (1H, s), 8.35 (1H, br. s.), 7.64 (1H, d, J=8.8 Hz), 7.40 (1H, dd, J=8.6, 1.6 Hz), 6.77 (2 H, s), 5.27 (1H, s), 4.21 (2H, br. s.), 3.99 (3H, s), 3.12 (3H, s), 1.85-1.98 (4H, m), 1.64-1.83 (4H, m). LCMS-ESI (POS), M/Z, M+1: Found 402.4.

### Example 245

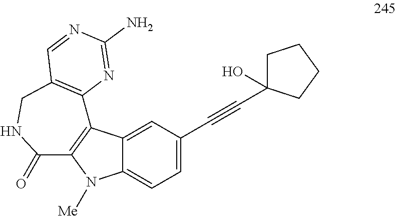

### 2-Amino-1′-((1-hydroxycyclopentyl)ethynyl)-8-methyl-5,8-dihydropyrimido[4′,′:5,6]azepino[3,4-b]indol-7(6H)-one

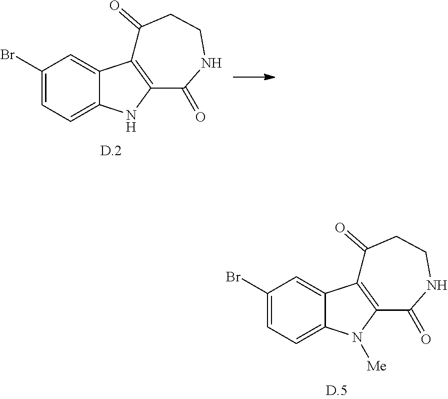

7-Bromo-10-methyl-3,4-dihydroazepino[3,4-b]indole-1,5(2H,10H)-dione (D.5) was prepared in 89% yield from 7-bromo-3,4-dihydroazepino[3,4-b]indole-1,5(2H,10H)-dione D.2 via mono-alkylation with iodomethane using potassium carbonate in acetonitrile at reflux. ¹H NMR (400 MHz, DMSO-d₆) δ ppm 8.86 (1H, t, J=6.0 Hz), 8.42 (1H, d, J=2.2 Hz), 7.68 (1H, d, J=8.8 Hz), 7.52-7.58 (1H, m), 4.01 (3H, s), 3.37-3.47 (2H, m), 2.75-2.86 (2H, m).

LCMS-ESI (POS), M/Z, M+1: Found 307.3 and 309.3.

2-Amino-1′-((1-hydroxycyclopentyl)ethynyl)-8-methyl-5,8-dihydropyrimido[4′,5′:5,6]azepino[3,4-b]indol-7(6H)-one (245) was prepared from 7-bromo-10-methyl-3,4-dihydroazepino[3,4-b]indole-1,5(2H,10H)-dione D.5 using chemistry similar to that described in Example 243. ¹H NMR (400 MHz, DMSO-d₆) δ ppm 8.60 (1H, t, J=5.9 Hz), 8.54 (1H, d, J=1.1 Hz), 8.24 (1H, br. s.), 7.64 (1H, d, J=8.4 Hz), 7.40 (1H, dd, J=8.6, 1.6 Hz), 6.72 (2H, s), 5.27 (1H, s), 4.01 (3H, s), 3.91 (2H, d, J=5.9 Hz), 1.88-1.96 (4 H, m), 1.63-1.81 (4H, m). LCMS-ESI (POS), M/Z, M+1: Found 388.4.

### Example 246

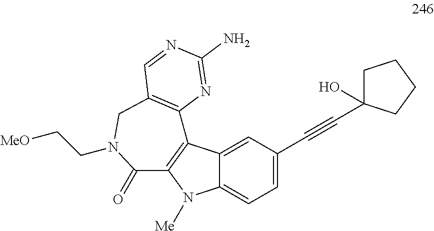

### 2-Amino-1′-((1-hydroxycyclopentyl)ethynyl)-6-(2-methoxyethyl)-8-methyl-5,8-dihydropyrimido[4′,′:5,6]azepino[3,4-b]indol-7(6H)-one

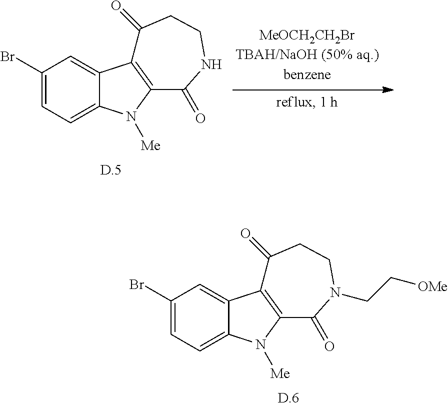

7-Bromo-2-(2-methoxyethyl)-10-methyl-3,4-dihydroazepino[3,4-b]indole-1,5(2H,10H)-dione (D.6) was prepared from 7-bromo-10-methyl-3,4-dihydroazepino[3,4-b]indole-1,5(2H,10H)-dione D.5 via alkylation under phase-transfer conditions with 1-bromo-2-methoxyethane. ¹H NMR (400 MHz, DMSO-d₆) δ ppm 8.36 (1H, d, J=1.5 Hz), 7.68 (1H, d, J=9.1 Hz), 7.51-7.58 (1H, m), 3.98 (3H, s), 3.78 (4H, br. s.), 3.57 (2H, t, J=5.5 Hz), 3.28 (3 H, s), 2.83-2.91 (2H, m). LCMS-ESI (POS), M/Z, M+1: Found 365.3 and 367.3.

2-Amino-1′-((1-hydroxycyclopentyl)ethynyl)-6-(2-methoxyethyl)-8-methyl-5,8-dihydropyrimido[4′,5′:5,6]azepino[3,4-b]indol-7(6H)-one (246) was prepared from 7-bromo-2-(2-methoxyethyl)-10-methyl-3,4-dihydroazepino[3,4-b]indole-1,5(2H,10H)-dione D.6 using chemistry similar to that described in Example 243. ¹H NMR (400 MHz, DMSO-d₆) δ ppm 8.52 (1H, d, J=1.5 Hz), 8.34 (1H, s), 7.64 (1H, d, J=8.8 Hz), 7.40 (1H, dd, J=8.6, 1.6 Hz), 6.74 (2H, s), 5.27 (1H, s), 4.23 (2H, s), 3.99 (3H, s), 3.43-3.56 (2H, m), 3.24-3.28 (2 H, m), 3.22 (3H, s), 1.87-1.98 (4H, m), 1.66-1.82 (4H, m). LCMS-ESI (POS), M/Z, M+1: Found 446.4.

### Example 247

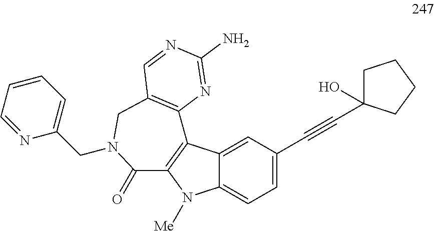

2-Amino-1′-((1-hydroxycyclopentyl)ethynyl)-8-methyl-6-(2-pyridinylmethyl)-5,8-dihydropyrimido[4′,′:5,6]azepino[3,4-b]indol-7(6H)-one (247) was prepared from 7-bromo-10-methyl-3,4-dihydroazepino[3,4-b]indole-1,5(2H,10H)-dione (D.5) using chemistry similar to that described in Example 246. ¹H NMR (500 MHz, DMSO-d₆) δ ppm 8.61 (1H, s), 8.52 (1H, s), 8.49 (1H, d, J=4.4 Hz), 8.14 (1H, s), 7.76 (1H, d, J=7.8 Hz), 7.66 (1H, d, J=8.8 Hz), 7.41 (1H, dd, J=8.8, 1.5 Hz), 7.36 (1H, dd, J=7.8, 4.9 Hz), 6.75 (2H, br. s.), 5.27 (1H, s), 4.81 (2H, s), 4.27 (2H, br. s.), 4.03 (3H, s). LCMS-ESI (POS), M/Z, M+1: Found 479.4.

### Example 248

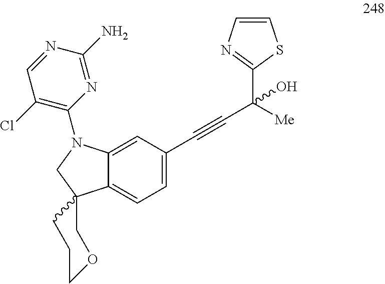

4-(1-(2-Amino-5-chloro-4-pyrimidinyl)-1,2,5′,6′-tetrahydro-4′H-spiro[indole-3,3′-pyran]-6-yl)-2-(1,3-thiazol-2-yl)-3-butyn-2-ol (248) was prepared using chemistry similar to that described in Example 180. (an off-white solid) LCMS-ESI (POS), M/Z, M+1: Found 468.1.

### Example 249

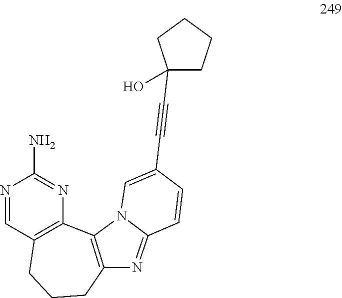

### 1-((2-amino-6,7-dihydro-5H-pyrido[1″,2″:1′,2′]imidazo[4′,′:6,7]cyclohepta[1,2-d]pyrimidin-11-yl)ethynyl)cyclopentanol (249)

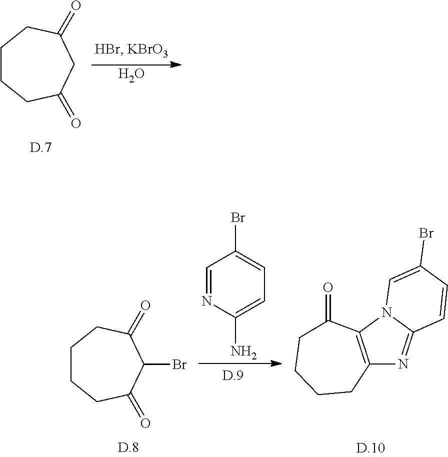

2-Bromo-6,7,8,9-tetrahydro-10H-cyclohepta[4,5]imidazo[1,2-a]pyridin-10-one (D.10): To a solution of 1,3-cycloheptanedione D.7 (5.04 g, 40.0 mmol) in water (20 mL) were added 48% HBr (4.53 mL, 40.0 mmol) and potassium bromate (2.23 g, 13.3 mmol) in water (20 mL). The mixture was stirred at room temperature for 30 min. The formed oil was extracted using ethyl ether and organic layer was washed with brine then dried. The solvent was evaporated to give compound D.8 as a brown oil. The brown oil was dissolved in ethylene glycol dimethyl ether (40 mL) and 5-bromopyridin-2-amine D.9 (5.16 g, 30 mmol) was added. The mixture thus obtained was stirred at 90° C. for 1 hr then cooled to room temperature. To the reaction mixture was added sodium bicarbonate (5.04 g, 60 mmol) and the mixture was stirred at 90° C. for additional 1 hr. The reaction mixture was poured into water (300 mL) and extracted with dichloromethane. The organic layer was washed with brine and dried. The solvent was evaporated under vacuum and the residue was purified by flash chromatography on silica gel eluting with 30% ethyl acetate in hexane to give the titled compound D.10 as a white solid (4.74 g, 43%) ¹H NMR (500 MHz, CDCl₃) δ 9.87 (1H, d, J=5.0 Hz), 7.54 (1H, d, J=5.0 Hz), 7.53 (1H, s), 3.22 (2H, m), 2.86 (2H, m), 2.05-1.97 (5H, m) ppm; LCMS-ESI (POS), M/Z, M+1: Found 279.0, Calculated 279.0.

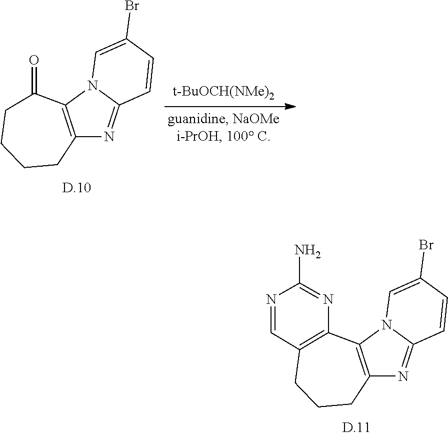

11-Bromo-6,7-dihydro-5H-pyrido[1″,2″:1′,2′]imidazo[4′,′:6,7]cyclohepta[1,2-d]pyrimidin-2-amine (D.11): A mixture of 2-bromo-6,7,8,9-tetrahydro-10H-cyclohepta[4,5]imidazo[1,2-a]pyridin-10-one (D.10) and t-butoxybis(dimethylamine)-methane (261 mg, 1.5 mmol) was heated at 100° C. for 2 hr. To the reaction mixture were added guanidine hydrochloride (287 mg, 3 mmol), 30% sodium methoxide (375 uL, 2 mmol) and 1-propanol (2 mL). The mixture thus obtained was stirred at 85° C. for 20 hr. The mixture was concentrated and the residue was purified by flash chromatography on silica gel eluting with 2% methanol in dichloromethane to give the titled compound D.11 as light yellow solid (256 mg, 78%) ¹H NMR (500 MHz, CDCl₃) δ 1025 (1H, s), 8.09 (1H, s), 7.50 (1H, d, J=10 Hz), 7.50 (1H, d, J=10 Hz), 5.16 (2H, brs), 3.34 (2H, t, J=8.4 Hz), 2.75 (2H, t, J=6.5 Hz), 2.08 (2H, m) ppm; LCMS-ESI (POS), M/Z, M+1: Found 330.1, Calculated 330.0.

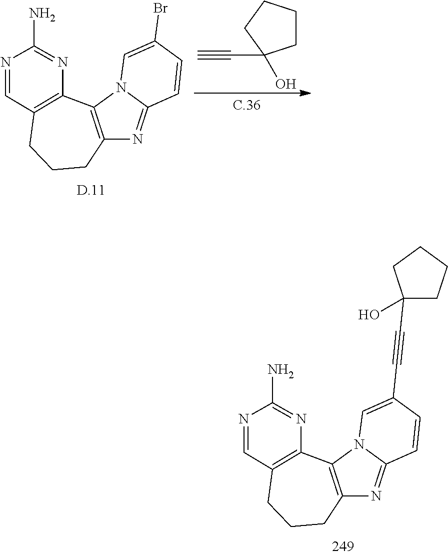

1-((2-Amino-6,7-dihydro-5H-pyrido[1″,2″:1′,2′]imidazo[4′,′:6,7]cyclohepta[1,2-d]pyrimidin-11-yl)ethynyl)cyclopentanol (249): To a degassed solution of 11-bromo-6,7-dihydro-5H-pyrido[1″,2″:1′,2′]imidazo[4′,′:6,7]cyclohepta[1,2-d]pyrimidin-2-amine (D.11) in DMF (1 mL) were added triethylamine (500 uL), CuI (4 mg, 0.02 mmol), and 1-ethylnyl-cyclopentanol C.36 (88 mg, 0.8 mmol, 77.5 uL). The mixture thus obtained was stirred at 80° C. under argon atmosphere overnight. The mixture was concentrated and the residue was purified by flash chromatography on silica gel eluting with dichloromethane/methanol/NH₄OH (100:10:1) to give 1-((2-amino-6,7-dihydro-5H-pyrido[1″,2″:1′,2′]imidazo-[4′,′:6,7]cyclohepta[1,2-d]pyrimidin-11-yl)ethynyl)cyclopentanol (249) as a light yellow solid (52 mg, 79%) LCMS-ESI (POS), M/Z, M+1: Found 361.1, Calculated 361.2.

### Example 250

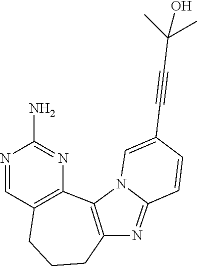

4-(2-Amino-6,7-dihydro-5H-pyrido[1″,2″:1′,2′]imidazo[4′,′:6,7]cyclohepta[1,2-d]pyrimidin-11-yl)-2-methyl-3-butyn-2-ol (250) was prepared from components D.11 and C.35 using chemistry similar to that described in example 249. ¹H NMR (500 MHz, methanol-d₄) δ 10.27 (1H, s), 8.06 (1H, s), 7.75 (1H, d, J=10 Hz), 7.37 (1H, d, J=10 Hz), 3.38 (2H, t, 8.2 Hz), 2.85 (2H, t, J=6.4 Hz), 2.13 (2H, m), 1.62 (6H, s) ppm; LCMS-ESI (POS), M/Z, M+1: Found 334.3, Calculated 334.2.

### Example 251

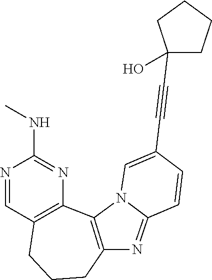

1-((2-(methylamino)-6,7-dihydro-5H-pyrido[1″,2″:1′,2′]imidazo[4′,5′:6,7]-cyclohepta[1,2-d]pyrimidin-11-yl)ethynyl)cyclopentanol (251) was prepared using chemistry similar to that described in example 249. ¹H NMR (500 MHz, methanol-d₄) δ 10.27 (1H, s), 8.10 (1H, s), 7.80 (1H, d, J=12 Hz), 7.77 (1H, d, J=12 Hz), 3.38 (2H, t, 8.2 Hz), 3.15 (3H, s), 2.85 (2H, t, J=6.4 Hz), 2.13 (2H, m), 2.05 (4H, m), 1.80-1.62 (4H, m) ppm; LCMS-ESI (POS), M/Z, M+1: Found 374.0, Calculated 374.2.

### Example 252

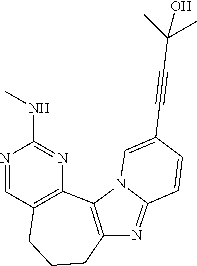

2-methyl-4-(2-(methylamino)-6,7-dihydro-5H-pyrido[1″,2″:1′,2′]imidazo[4′,′:6,7]-cyclohepta[1,2-d]pyrimidin-11-yl)-3-butyn-2-ol (252) was prepared using chemistry similar to that described in example 249. ¹H NMR (500 MHz, methanol-d₄) δ 10.27 (1H, s), 8.10 (1H, s), 7.81-7.77 (2H, m), 3.38 (2H, m), 3.31 (3H, s), 3.16 (2H, m), 2.88 (2H, m), 2.14 (2H, m), 1.60 (6H, s) ppm; LCMS-ESI (POS), M/Z, M+1: Found 348.1, Calculated 348.2.

### Example 253

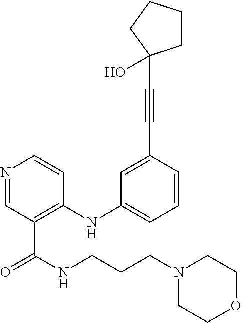

### 4-((3-((1-hydroxycyclopentyl)ethynyl)phenyl)amino)-N-(3-(4-morpholinyl)propyl)-3-pyridinecarboxamide

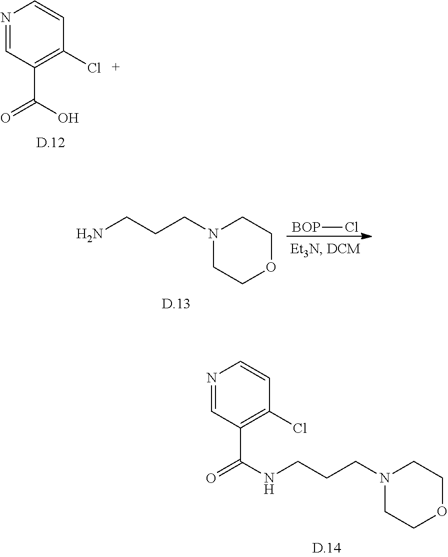

4-Chloro-N-(3-morpholinopropyl)nicotinamide (D.14): A solution of 4-chloronicotinic acid D.12 (157 mg, 1.0 mmol), N-(3-aminopropyl)morpholine D.13 (173 mg, 1.2 mmol), BOP-Cl (255 mg, 1.0 mmol), and triethylamine (140 uL, 1.0 mmol) in dichloromethane (2 mL) was stirred at room temperature for 2 hr. The reaction mixture was diluted with dichloromethane (10 mL) and washed with water. The organic layer was concentrated and the residue was purified by flash chromatography on silica gel eluting with 2.5% methanol in dichloromethane to give 4-chloro-N-(3-morpholinopropyl)nicotinamide (D.14) as a colorless oil (163 mg, 58%). ¹H NMR (500 MHz, methanol-d₄) δ 8.77 (1H, s), 8.55 (1H, d, J=7.0 Hz), 7.88 (1H, br s), 7.37 (1H, d, J=7.0 Hz), 3.62-3.56 (6H, m), 2.56 (1H, t, J=7.5 Hz), 2.48 (4H, m), 1.81 (2H, q, J=7.5 Hz) ppm; LCMS-ESI (POS), M/Z, M+1: Found 284.1, Calculated 281.1.

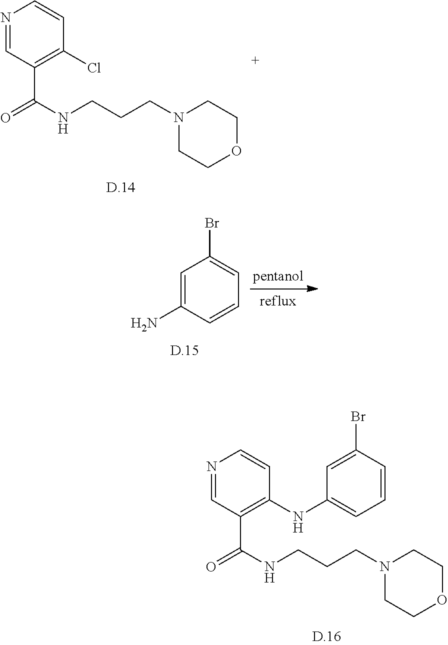

### 4-((3-Bromophenyl)amino)-N-(3-(4-morpholinyl)propyl)-3-pyridinecarboxamide (D.16)

A solution of D.14 (78 mg, 0.276 mmol) and 3-bromoaniline D.15 (172 mg, 1.0 mmol) in pentanol (2 mL) was refluxed for 3 hr. The reaction mixture was concentrated and the residue was purified by flash chromatography on silica gel eluting with dichloromethane/methanol/NH4OH (100:10:1) to give D.16 as a colorless oil (80 mg, 70%) ¹H NMR (500 MHz, methanol-d₄) δ 11.62 (1H, s), 9.14 (1H, s), 9.00 (1H, s), 8.03 (1H, d, J=8.9 Hz), 7.54 (1H, dd, J=10 Hz, J=2Hz), 7.46 (1H, t, J=2Hz), 7.39 (1H, t, J=10 Hz), 7.24 (1H, dd, J=10 Hz, J=2Hz), 7.10 (1H, d, J=8.9 Hz), 4.02-3.89 (4H, m), 3.58-3.52 (4H, m), 3.25 (2H, m), 2.94 (2H, m), 2.15 (2H, m) ppm; LCMS-ESI (POS), M/Z, M+1: Found 419.0, Calculated 419.1.

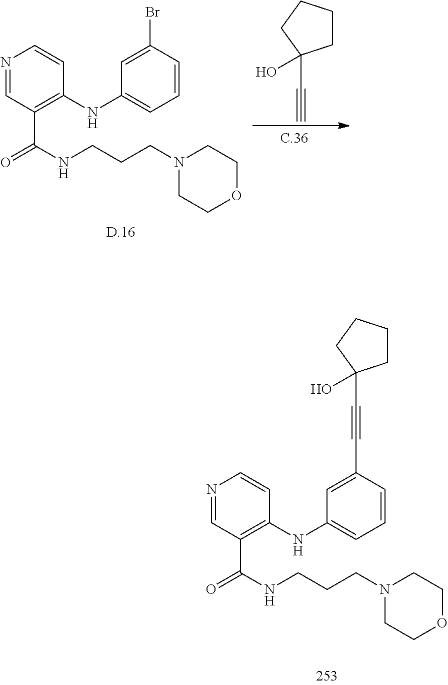

4-((3-((1-Hydroxycyclopentyl)ethynyl)phenyl)amino)-N-(3-(4-morpholinyl)propyl)-3-pyridinecarboxamide (253) was prepared from components D.16 and C.36 using chemistry similar to that described in example 249. ¹H NMR (500 MHz, methanol-d₄) δ 11.55 (1H, s), 9.20 (1H, s), 8.94 (1H, s), 8.00 (1H, d, J=9.0 Hz), 7.45-7.44 (2H, m), 7.34 (1H, s), 7.22 (1H, m), 6.98 (1H, d, J=9.0 Hz), 4.00-3.95 (4H, m), 3.60-3.54 (4H, m), 3.26 (2H, m), 2.93 (2H, m), 2.15 (2H, m), 2.08-2.03 (4H, m), 1.90-1.79 (4H, m) ppm; LCMS-ESI (POS), M/Z, M+1: Found 449.3, Calculated 449.2.

### Example 254

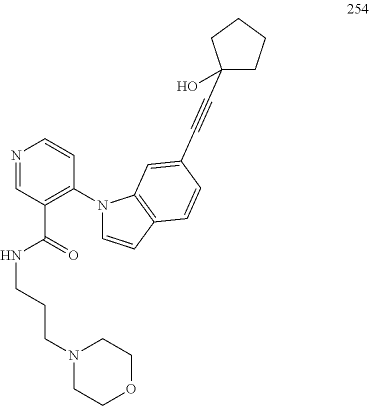

### 4-(6-((1-hydroxycyclopentypethynyl)-1H-indol-1-yl)-N-(3-(4-morpholinyl)propyl)-3-pyridinecarboxamide (254)

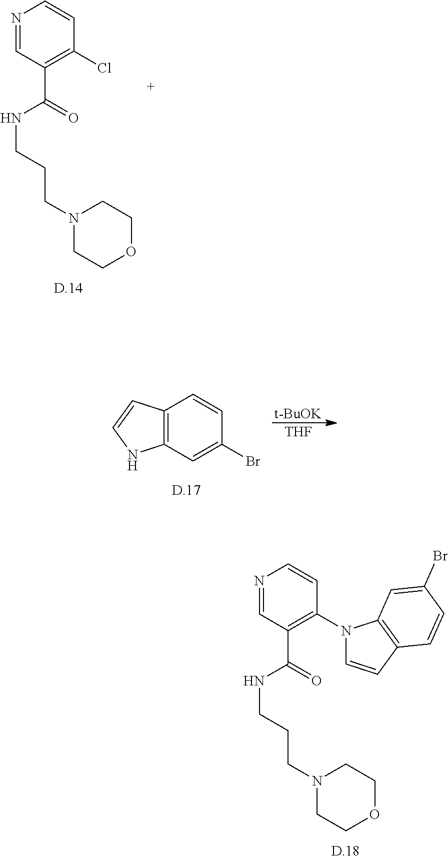

### 4-(6-Bromo-1H-indol-1-yl)-N-(3-(4-morpholinyl)propyl)-3-pyridinecarboxamide (D.18)

To a solution of D.14 (134 mg, 0.473 mmol) and 6-bromoindole D.17 (186 mg, 0.95 mmol) in THF (2 mL) was added potassium t-butoxide (112 mg, 1.0 mmol). The resulting mixture was stirred at room temperature for 12 hr, pH adjusted to 8 with saturated aqueous ammonium chloride, then extracted with dichloromethane. The organic layer was dried and concentrated. The residue was purified by flash chromatography on silica gel eluting with 5% methanol in dichloromethane to give 4-(6-bromo-1H-indol-1-yl)-N-(3-(4-morpholinyl)propyl)-3-pyridinecarboxamide (D.18) as white solid (124 mg, 61%) LCMS-ESI (POS), M/Z, M+1: Found 443.2, Calculated 443.1.

4-(6-((1-Hydroxycyclopentyl)ethynyl)-1H-indol-1-yl)-N-(3-(4-morpholinyl)propyl)-3-pyridinecarboxamide (254) was prepared from components D.18 and C.36 using chemistry similar to that described in example 249. ¹H NMR (500 MHz, methanol-d₄) δ 11.00 (1H, s), 9.31 (1H, s), 9.00 (1H, s), 8.87 (1H, m), 7.81 (1H, m), 7.57 (2H, m), 7.42 (1H, m), 6.81 (1H, m), 4.01-3.95 (4H, m), 3.44-3.41 (4H, m), 3.01 (2H, m), 2.82 (2H, m), 2.11-2.02 (6H, m), 1.86-1.77 (4H, m) ppm; LCMS-ESI (POS), M/Z, M+1: Found 473.4, Calculated 473.3.

### Example 255

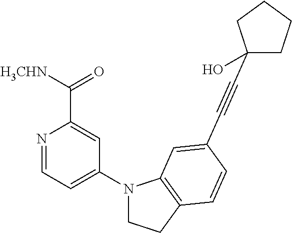

### 4-(6-((1-hydroxycyclopentyl)ethynyl)-2,3-dihydro-1H-indol-1-yl)-N-methyl-2-pyridinecarboxamide (255)

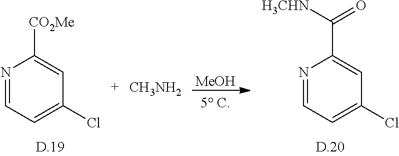

4-Chloro-N-methylpicolinamide (D.20): A solution of methyl 4-chloropicolinate (D.19) (1.72 g, 10 mmol) in methanol (10 mL) was treated with 2 M methylamine in methanol (20 mL) at 0° C. The mixture was stirred at 5° C. for 3 hr. The reaction mixture was concentrated and the residue was dissolved in ethyl acetate and washed with brine, dried, concentrated to give 4-chloro-N-methylpicolinamide (D.20) as colorless oil (1.70 g, 99%). LCMS-ESI (POS), M/Z, M+1: Found 171.1, Calculated 171.0.

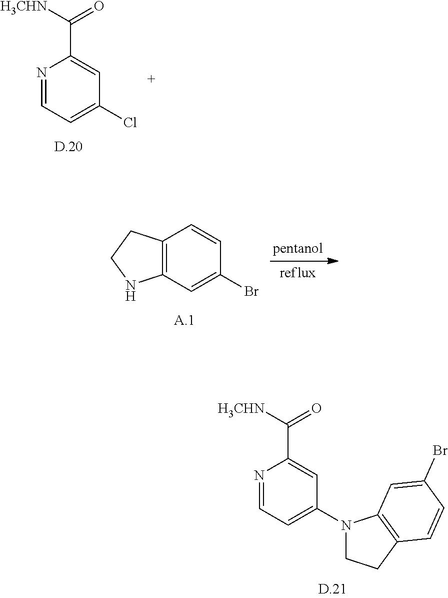

4-(6-Bromo-2,3-dihydro-1H-indol-1-yl)-N-methyl-2-pyridinecarboxamide (D.21): A mixture of D.20 (340 mg, 2.0 mmol) and 6-bromoindoline A.1 (396 mg, 2.0 mmol) in 1-pentanol (2 mL) was refluxed for 16 hr. The reaction mixture was concentrated and the residue was purified by flash chromatography on silica gel eluting with 2.5% methanol in dichloromethane to give 4-(6-bromo-2,3-dihydro-1H-indol-1-yl)-N-methyl-2-pyridinecarboxamide (D.21) as light brown solid (620 mg, 98%). ¹H NMR (500 MHz, methanol-d₄) δ 8.34 (2H, m), 7.88 (1H, s), 7.50 (1H, s), 7.25 (1H, m), 7.11 (1H, d, J=7.5 Hz), 7.07 (1H, d, J=7.5 Hz), 4.13 (2H, t, J=10.2 Hz), 3.16 (2H, t, J=10.2 Hz), 3.05 (3H, d, J=6.3 Hz) ppm; LCMS-ESI (POS), M/Z, M+1: Found 332.1, Calculated 332.0.

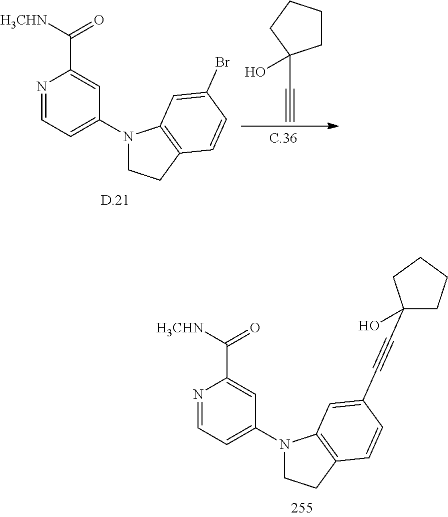

4-(6-((1-hydroxycyclopentyl)ethynyl)-2,3-dihydro-1H-indol-1-yl)-N-methyl-2-pyridinecarboxamide(255): Using the procedure described for the preparation of 249, 125 mg (69%) of 255 was obtained as a yellow solid from 166 mg (0.5 mmol) of D.21 and 220 mg (2.0 mmol) of 1-ethynyl-cyclopentanol C.36. ¹H NMR (500 MHz, methanol-d₄) δ 9.65 (1H, s), 8.35 (1H, s), 8.13 (1H, m), 7.54 (2H, m), 7.32 (1H, d, J=7.5 Hz), 7.24 (1H, d, J=7.5 Hz), 4.35 (2H, t, J=10 Hz), 3.30 (2H, t, J=10 Hz), 3.08 (3H, br s), 2.15-2.05 (4H, m), 1.95-1.75 (4H, m) ppm; LCMS-ESI (POS), M/Z, M+1: Found 362.2, Calculated 362.2.

### Example 256

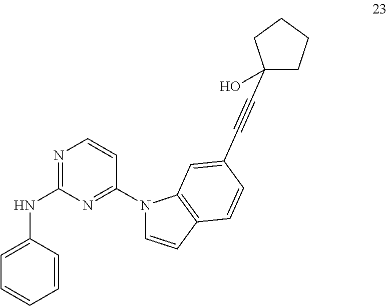

### 1-((1-(2-(phenylamino)-4-pyrimidinyl)-1H-indol-6-yl)ethynyl)cyclopentanol

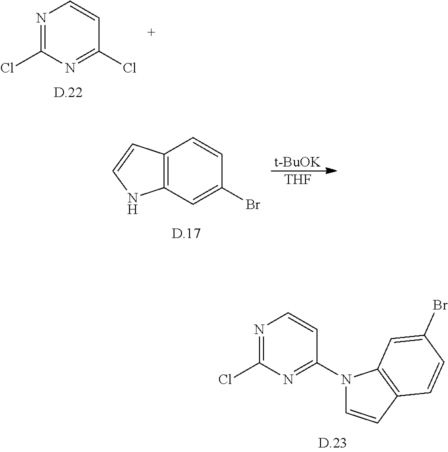

6-Bromo-1-(2-chloropyrimidin-4-yl)-1H-indole (D.23): To a solution of 6-bromoindole (D.17) (1.96 g, 10 mmol) in THF (20 mL) was added potassium t-butoxide (1.34 mg, 12 mmol) and stirred at room temperature for 1 hr. To the reaction mixture was added 2,4-dichloropyrimidine (D.22) (1.49 g, 10 mmol) and stirred at room temperature additional 2 hr. The reaction mixture was quenched with Sat's NH₄Cl and diluted with ethyl acetate, washed with brine. The organic layer was dried over MgSO₄ and concentrated. The residue was purified by flash chromatography on silica gel to give 6-bromo-1-(2-chloropyrimidin-4-yl)-1H-indole (D.23) as white solid (1.92 g, 62%).LCMS-ESI (POS), M/Z, M+1: Found 308.0, Calculated 308.0.

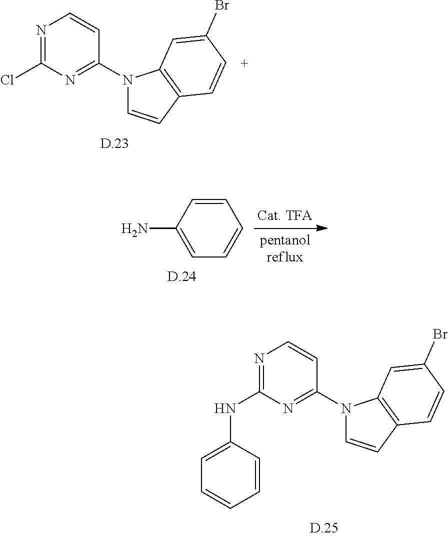

4-(6-Bromo-1H-indol-1-yl)-N-phenylpyrimidin-2-amine (D.25): A mixture of D.23 (170 mg, 0.55 mmol), aniline (102 mg, 1.10 mmol), and TFA (10 uL) in pentanol (2 mL) heated at 140° C. for 2 hr. The reaction mixture was cooled to room temperature and the precipitate was collected by filtration and the solid was washed with ethanol. The solid was re-crystallized in ethanol to give 4-(6-bromo-1H-indol-1-yl)-N-phenylpyrimidin-2-amine (D.25) as white needle shape crystal (150 mg, 75%). LCMS-ESI (POS), M/Z, M+1: Found 365.1, Calculated 365.0.

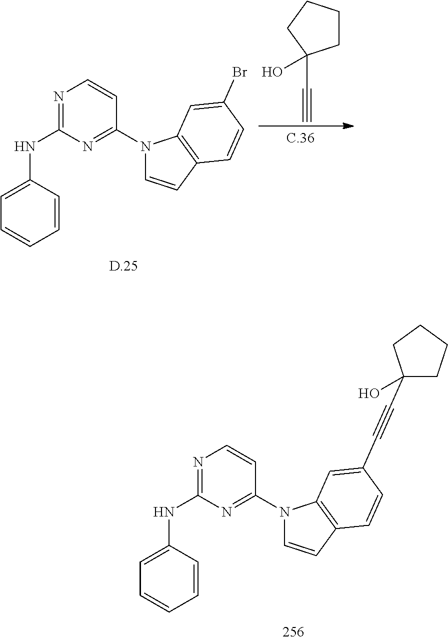

1-((1-(2-(Phenylamino)-4-pyrimidinyl)-1H-indol-6-yl)ethynyl)cyclopentanol (256): Using the procedure described for the preparation of 249, 90 mg (64%) of 256 was obtained as light yellow solid from 130 mg (0.356 mmol) of D.25 and 158 mg (1.44 mmol) of 1-ethynyl-cyclopentanol C.36. ¹H NMR (500 MHz, methanol-d₄) δ 8.95 (1H, s), 8.45 (1H, s), 8.25 (1H, d, J=7.5 Hz), 7.65 (2H, m), 7.55-7.44 (4H, m), 7.34 (1H, dd, J=8.2; 1.8 Hz), 7.20 (1H, dd, J=8.2; 7.5 Hz), 6.82 (1H, d, J=8.2 Hz), 6.75 (1H, d, J=5 Hz), 2.10-2.00 (4H, m), 1.95-1.75 (4H, m) ppm; LCMS-ESI (POS), M/Z, M+1: Found 395.2, Calculated 395.2.

### Example 257

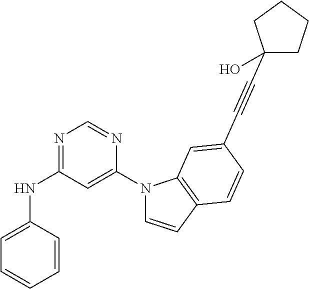

### 1-((1-(6-(phenylamino)-4-pyrimidinyl)-1H-indol-6-yl)ethynyl)cyclopentanol (257)

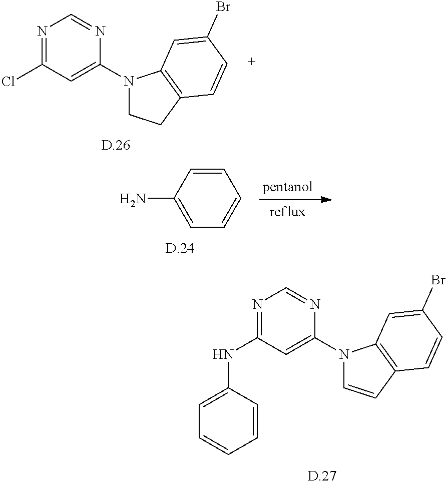

6-(6-Bromo-1H-indol-1-yl)-N-phenylpyrimidin-4-amine (D.27): A mixture of D.26 (331 mg, 1.0 mmol) and aniline (D.24) (200 mg, 2.0 mmol) in 1-pentanol (4 mL) was heated at 140° C. for 3 hr. The reaction mixture was cooled to room temperature and the precipitate was collected by filtration and the solid was recrystallized in ethanol to give compound D.27 as yellow needle-shaped crystals (350 mg, 96%). ¹H NMR (500 MHz, methanol-d₄) δ 8.62 (1H, s), 8.55 (1H, d, J=2.1 Hz), 7.58 (1H, d, J=4.9 Hz), 7.52-7.46 (3H, m), 7.42-7.31 (4H, m), 6.71 (1H, s), 6.68 (1H, d, J=4.6 Hz) ppm; LCMS-ESI (POS), M/Z, M+1: Found 365.1, Calculated 365.0.

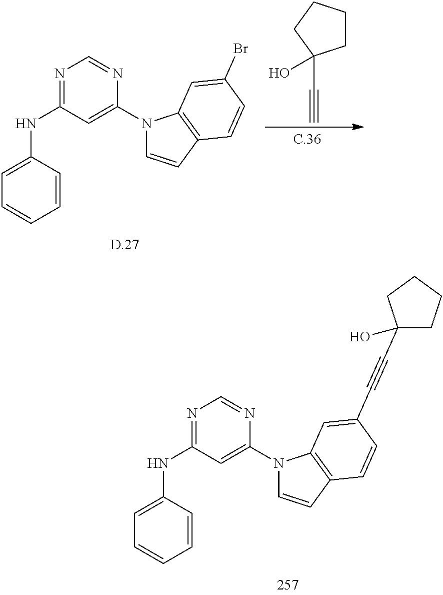

1-((1-(6-(Phenylamino)-4-pyrimidinyl)-1H-indol-6-yl)ethynyl)cyclopentanol: Using the procedure described for the preparation of example 249, 148 mg (78%) of compound 257 was obtained as a light yellow solid from 175 mg (0.48 mmol) of D.27 and 220 mg (2.0 mmol) of 1-ethynyl-cyclopentanol C.36. ¹H NMR (500 MHz, methanol-d₄) δ 8.59 (1H, s), 8.50 (1H, br s), 8.31 (1H, s), 7.63 (1H, d, J=4.6 Hz), 7.52-7.48 (3H, m), 7.43-7.40 (2H, m), 7.35-7.27 (2H, m), 6.71 (1H, s), 6.68 (1H, d, J=4.6 Hz), 2.15-2.05 (4H, m), 1.95-1.75 (4H, m) ppm; LCMS-ESI (POS), M/Z, M+1: Found 395.2, Calculated 395.2.

### Example 258

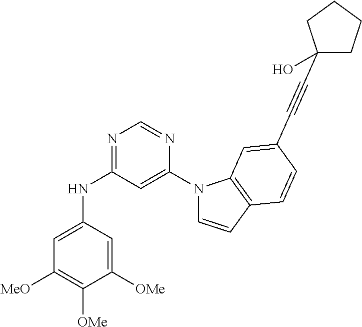

1-((1-(6-((3,4,5-tris(methyloxy)phenyl)amino)-4-pyrimidinyl)-1H-indol-6-yl)ethynyl)cyclopentanol (258)

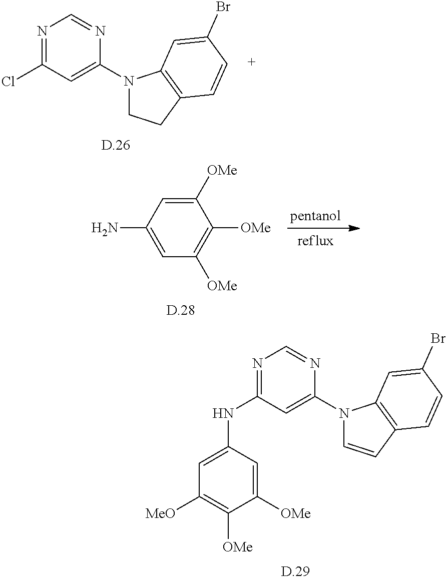

6-(6-Bromo-1H-indol-1-yl)-N-(3,4,5-tris(methyloxy)phenyl)-4-pyrimidinamine (D.29): A mixture of D.26 (331 mg, 1.0 mmol) and 3,4,5-trimethoxyaniline (366 mg, 2.0 mmol), and TFA (10 uL) in 1-pentanol (4 mL) was heated at 140° C. for 2 hr. The mixture was concentrated and the residue was purified by flash chromatography on silica gel eluting with 30% ethyl acetate in hexane to give compound D.29 as a light yellow solid (448 mg, 98%). LCMS-ESI (POS), M/Z, M+1: Found 455.0, Calculated 455.1.

1-((1-(6-((3,4,5-tris(methyloxy)phenyl)amino)-4-pyrimidinyl)-1H-indol-6-yl)ethynyl)cyclopentanol: Using the procedure described for the preparation of 249, 122 mg (50%) of compound 258 was obtained as yellow solid from 228 mg (0.5 mmol) of D.29 and 220 mg (2.0 mmol) of 1-ethynyl-cyclopentanol C.36. ¹H NMR (500 MHz, methanol-d₄) δ 8.58 (1H, s), 8.18 (1H, s), 7.85 (1H, br s), 7.77 (1H, d, J=7.5 Hz), 7.50 (1H, d, J=10 Hz), 7.25 (1H, d, J=10 Hz), 6.78 (1H, s), 6.66 (2H, m), 3.88 (9H, s), 2.15-2.08 (4H, m), 1.95-1.75 (4H, m) ppm; LCMS-ESI (POS), M/Z, M+1: Found 485.2, Calculated 485.2.

### Example 259

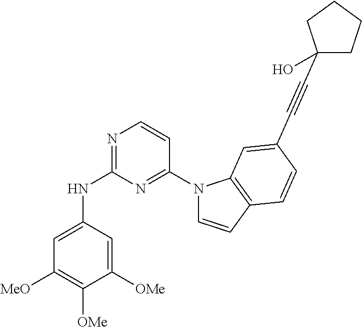

### 1-((1-(2-((3,4,5-tris(methyloxy)phenyl)amino)-4-pyrimidinyl)-1H-indol-6-yl)-ethynyl)cyclopentanol (259)

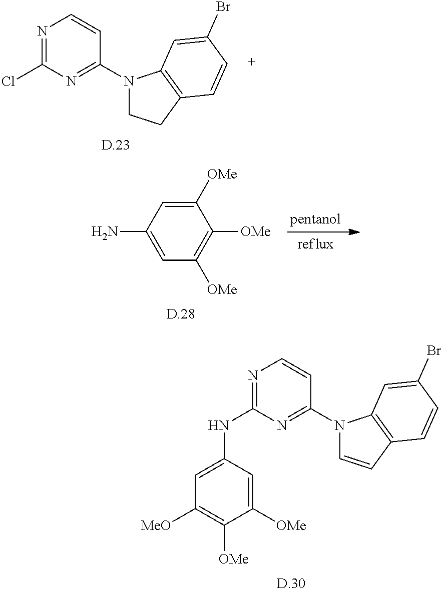

### 4-(6-Bromo-1H-indol-1-yl)-N-(3,4,5-tris(methyloxy)phenyl)-2-pyrimidinamine (D.30)

Using the procedure described for the preparation of D.29, 227 mg (77%) of D.30 was obtained as white solid from 200 mg (0.65 mmol) of D.23 and 238 mg (1.3 mmol) of 3,4,5-trimethoxyaniline. LCMS-ESI (POS), M/Z, M+1: Found 455.1, Calculated 455.1.

1-((1-(2-((3,4,5-Tris(methyloxy)phenyl)amino)-4-pyrimidinyl)-1H-indol-6-yl)-ethynyl)cyclopentanol (259): Using the procedure described for the preparation of 249, 15 mg (10%) of compound 259 was obtained as a yellow solid from 135 mg (0.3 mmol) of D.30 and 132 mg (1.2 mmol) of 1-ethynyl-cyclopentanol C.36. LCMS-ESI (POS), M/Z, M+1: Found 485.3, Calculated 485.2.

### Example 260

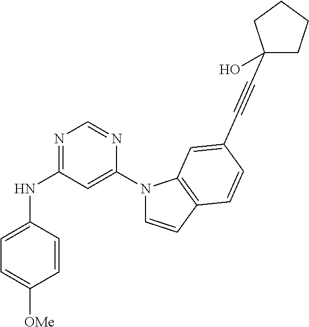

### 1-((1-(6-((4-(methyloxy)phenyl)amino)-4-pyrimidinyl)-1H-indol-6-yl)-ethynyl)cyclopentanol

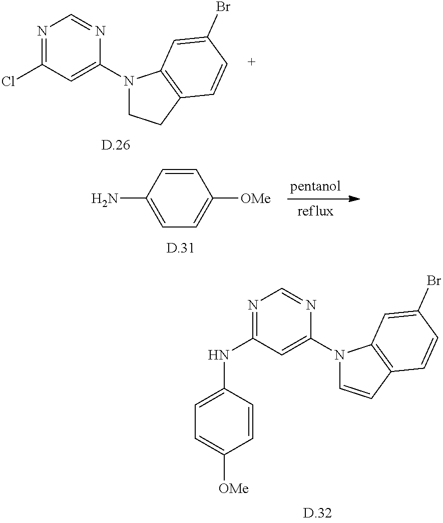

6-(6-Bromo-1H-indol-1-yl)-N-(4-(methyloxy)phenyl)-4-pyrimidinamine (D.32): Using the procedure described for the preparation of D.27, 356 mg (90%) of D.32 was obtained as a light yellow solid from 331 mg (1.0 mmol) of D.26 and 246 mg (2.0 mmol) of p-anisidine D.31. LCMS-ESI (POS), M/Z, M+1: Found 395.1, Calculated 395.0.

1-((1-(6-((4-(methyloxy)phenyl)amino)-4-pyrimidinyl)-1H-indol-6-yl)-ethynyl)-cyclopentanol(260): Using the procedure described for the preparation of 249, 95 mg (50%) of compound 260 was obtained as a light yellow solid from 178 mg (0.45 mmol) of D.32 and 200 mg (2.0 mmol) of 1-ethynyl-cyclopentanol C.36. ¹H NMR (500 MHz, methanol-d₄) δ 8.57 (1H, s), 8.25 (1H, s), 7.83 (1H, br s), 7.66 (1H, d, J=7.5 Hz), 7.52 (1H, d, J=10.2 Hz), 7.31-7.25 (3H, m), 7.05-6.98 (2H, m), 6.65 (1H, d, J=7.5 Hz), 6.56 (1H, s), 3.86 (3H, s), 2.15-2.05 (4H, m), 1.95-1.75 (4H, m) ppm; LCMS-ESI (POS), M/Z, M+1: Found 425.2, Calculated 425.2.

### Example 261

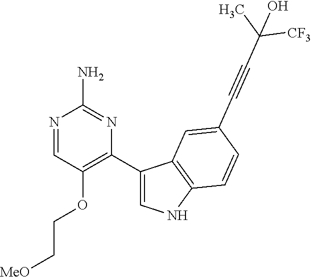

4-(3-(2-Amino-5-((2-(methyloxy)ethyl)oxy)-4-pyrimidinyl)-1H-indol-5-yl)-1,1,1-trifluoro-2-methyl-3-butyn-2-ol (261): Using the procedure described for the preparation of example 1, 32 mg (25%) of compound 261 was obtained as a yellow solid from 109 mg (0.3 mmol) of A.313 and 232 mg (1.2 mmol) of 57% 1,1,1-trifluoro-2-methylbut-3-yn-2-ol A.53 in THF. ¹H NMR (500 MHz, methanol-d₄) δ 12.45 (1H, s), 8.90 (1H, s), 8.70 (1H, s), 8.02 (1H, s), 7.52 (1H, d, J=8.2 Hz), 7.35 (1H, d, J=8.2 Hz), 7.00 (1H, br s), 4.25 (2H, m), 3.75 (2H, m), 3.38 (3H, s), 2.51 (3H, m) ppm; LCMS-ESI (POS), M/Z, M+1: Found 421.1, Calculated 421.1.

### Example 262

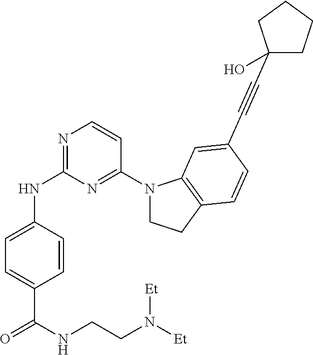

### N-(2-(diethylamino)ethyl)-4-((4-(6-((1-hydroxycyclopentyl)ethynyl)-2,3-dihydro-1H-indol-1-yl)-2-pyrimidinyl)amino)benzamide (262)

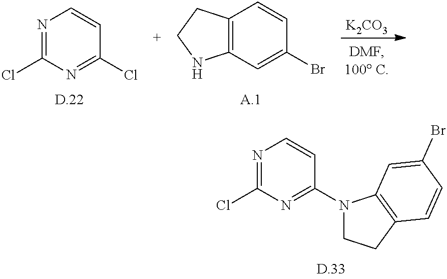

6-Bromo-1-(2-chloro-4-pyrimidinyl)-2,3-dihydro-1H-indole (D.33) To a hot solution of 2,4-dichloropyrimidine D.22 (4.5 g, 30 mmol) in DMF (20 mL) were added potassium carbonate (2.76 g, 20 mL) and 6-bromoindoline A.1 (3.98 g, 20 mmol). The resulting mixture was stirred at 100° C. overnight. The mixture was cooled to room temperature and poured into water (100 mL) and the precipitate was collected by filtration and washed with methanol to give 6-bromo-1-(2-chloro-4-pyrimidinyl)-2,3-dihydro-1H-indole (D.33) as a light yellow solid (4.45 g, 72%). LCMS-ESI (POS), M/Z, M+1: Found 310.0, Calculated 310.0.

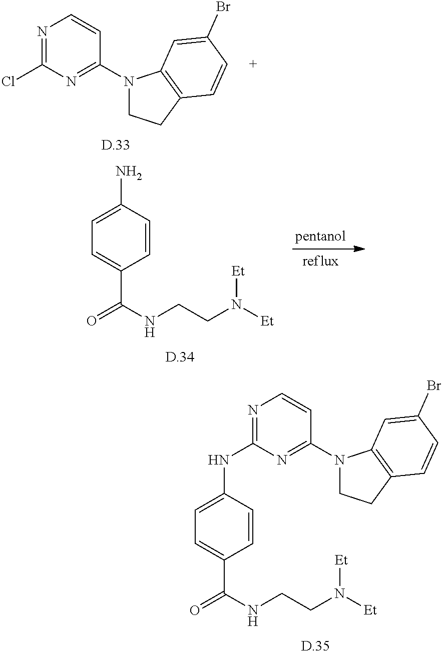

4-((4-(6-Bromo-2,3-dihydro-1H-indol-1-yl)-2-pyrimidinyl)amino)-N-(2-(diethylamino)ethyl)benzamide(D.35): Using the procedure described for the preparation of D.29, 195 mg (77%) of D.35 was obtained as a white solid from 155 mg (0.5 mmol) of D.33 and 271 mg (1.0 mmol) of procainamide hydrochloride D.34 . ¹H NMR (500 MHz, methanol-d₄) δ 9.62 (1H, s), 8.54 (1H, s), 8.24 (1H, d, J=7.2 Hz), 8.19 (1H, br s), 7.81 (2H, d, J=10.5 Hz), 7.77 (2H, d, J=10.5 Hz), 7.18 (1H, d, J=9.5 Hz), 7.10 (1H, d, J=10.5 Hz), 6.35 (1H, d, J=7.2 Hz), 4.07 (2H, t, J=10.5 Hz), 3.32 (4H, m), 3.16 (2H, t, J=10.5 Hz), 2.56-2.50 (5H, m), 0.99 (6H, t, J=7.5 Hz) ppm; LCMS-ESI (POS), M/Z, M+1: Found 509.2, Calculated 509.2.

N-(2-(diethylamino)ethyl)-4-((4-(6-((1-hydroxycyclopentyl)ethynyl)-2,3-dihydro-1H-indol-1-yl)-2-pyrimidinyl)amino)benzamide (262): Using the procedure described for the preparation of 249, 68 mg (81%) of 262 was obtained as yellow solid from 80 mg (0.157 mmol) of D.35 and 69 mg (0.628 mmol) of 1-ethynylcyclopentanol C.36. ¹H NMR (500 MHz, methanol-d₄) δ 8.51 (1H, s), 8.23 (1H, d, J=7.4 Hz), 7.99 (1H, d, J=10.7 Hz), 7.80 (1H, d, J=10.7 Hz), 7.69 (1H, s), 7.36 (1H, br s), 7.20 (1H, d, J=8.5 Hz), 7.16 (1H, d, J=8.5 Hz), 6.21 (1H, d, J=7.4 Hz), 4.10 (2H, t, J=10.5 Hz), 3.59 (2H, q, J=4.0 Hz), 3.29 (2H, t, J=10.5 Hz), 2.76 (2H, t, J=4.0 Hz), 2.69 (4H, q, J=9.1 Hz), 2.10 (4H, m), 1.95-1.75 (4H, m), 1.16 (6H, t, J=9.1 Hz) ppm; LCMS-ESI (POS), M/Z, M+1: Found 539.4, Calculated 539.3.

### Example 263

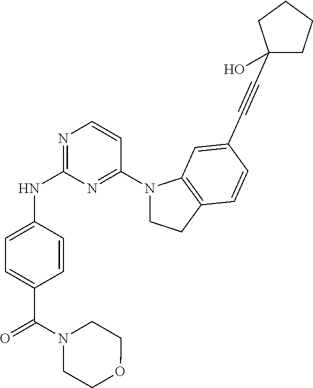

### 1-((1-(2-((4-(4-morpholinylcarbonyl)phenyl)amino)-4-pyrimidinyl)-2,3-dihydro-1H-indol-6-yl)ethynyl)cyclopentanol (263)

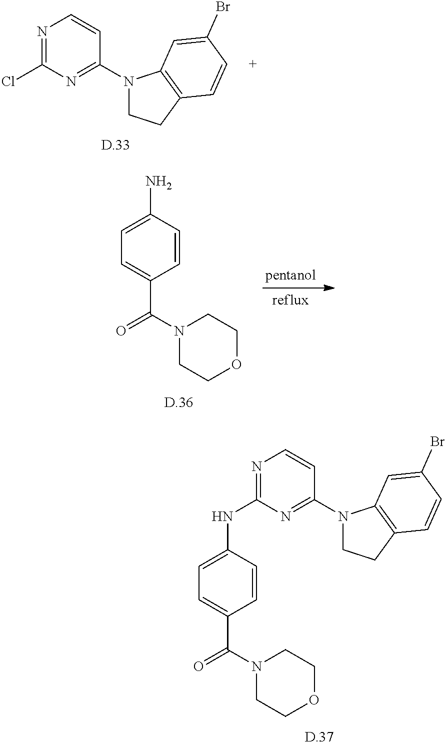

### (4-(4-(6-Bromoindolin-1-yl)pyrimidin-2-ylamino)phenyl)(morpholino)methanone (D.37)

Using the procedure described for the preparation of compound D.29, 212 mg (88%) of D.37 was obtained as white solid from 155 mg (0.5 mmol) of D.33 and 155 mg (0.75 mmol) of 4-aminophenyl(morpholino)methanone D.36. ¹H NMR (500 MHz, methanol-d₄) δ 9.70 (1H, s), 8.65 (1H, s), 8.33 (1H, d, J=6.8 Hz), 7.88 (1H, d, J=9.8 Hz), 7.48 (1H, d, J=9.8 Hz), 7.26 (1H, d, J=8.2 Hz), 7.19 (1H, d, J=8.2 Hz), 6.43 (1H, d, J=6.8 Hz), 4.16 (2H, t, J=10.0 Hz), 3.70 (4H, m), 3.62 (4H, m), 3.25 (2H, m) ppm; LCMS-ESI (POS), M/Z, M+1: Found 480.1, Calculated 480.1.

1-((1-(2-((4-(4-Morpholinylcarbonyl)phenyl)amino)-4-pyrimidinyl)-2,3-dihydro-1H-indol-6-yl)ethynyl)cyclopentanol (263): Using the procedure described for the preparation of 249, 60 mg (47%) of 263 was obtained as yellow solid from 120 mg (0.25 mmol) of D.37 and 110 mg (1.0 mmol) of 1-ethynylcyclopentanol C.36. ¹H NMR (500 MHz, methanol-d₄) δ 8.98 (1H, s), 7.83 (1H, d, J=8.4 Hz), 7.58 (1H, d, J=8.4 Hz), 7.22 (1H, d, J=8 Hz), 7.16 (1H, d, J=8 Hz), 6.25 (1H, br s), 4.15 (2H, t, J=7 Hz), 3.80-3.65 (8H, m), 3.33 (2H, t, J=7 Hz), 2.20-1.75 (8H, m) ppm; LCMS-ESI (POS), M/Z, M+1: Found 510.3, Calculated 510.2.

### Example 264

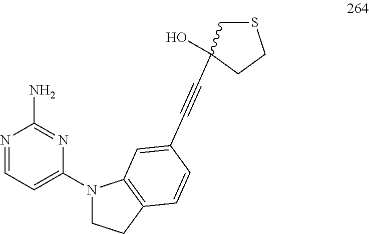

3-((1-(2-amino-4-pyrimidinyl)-2,3-dihydro-tH-indol-6-yl)ethynyl)tetrahydro-3-thiopheneol (264): Using the procedure described for the preparation of 249, 34 mg (40%) of 264 was obtained as a yellow solid from 73 mg (0.25 mmol) of A.95 and 64 mg (0.5 mmol) of 3-ethynyltetrahydrothiophen-3-ol C.43. ¹H NMR (500 MHz, methanol-d₄) δδ 8.62 (1H, s), 8.05 (1H, d, J=9.0 Hz), 7.25 (1H, d, J=10.0 Hz), 7.15 (1H, d, J=10.0 Hz), 6.29 (1H, d, J=9.0 Hz), 4.15 (2H, t, J=10.5 Hz), 3.29 (2H, t, J=10.5 Hz), 3.15-3.05 (4H, m), 2.50 (1H, m), 2.30 (1H, m) ppm; LCMS-ESI (POS), M/Z, M+1: Found 367.2, Calculated 367.2.

### Example 265

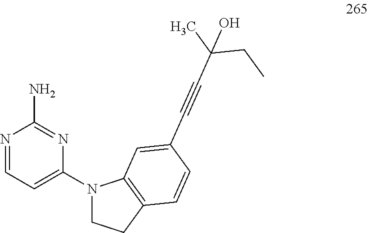

### 1-(1-(2-Amino-4-pyrimidinyl)-2,3-dihydro-1H-indol-6-yl)-3-methyl-1-pentyn-3-ol (265)

Using the procedure described for the preparation of 249, 102 mg (66%) of 265 was obtained as a yellow solid from 145 mg (0.5 mmol) of A.95 and 196 mg (2.0 mmol) of 3-methyl-1-pentyn-3-ol C.44. ¹H NMR (500 MHz, methanol-d₄) δ 8.48 (1H, s), 8.08 (1H, d, J=7.0 Hz), 7.22 (1H, d, J=10.0 Hz), 7.05 (1H, d, J=10.0 Hz), 6.40 (2H, s), 6.13 (1H, d, J=7.0 Hz), 5.34 (1H, s), 4.06 (2H, t, J=10.5 Hz), 3.25 (2H, t, J=10.5 Hz), 1.75 (2H, m), 1.54 (3H, s), 1.11 (3H, t, J=7.4 Hz) ppm; LCMS-ESI (POS), M/Z, M+1: Found 309.2, Calculated 309.2.

### Example 266

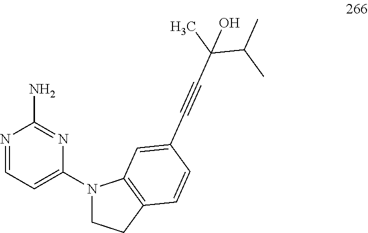

1-(1-(2-Amino-4-pyrimidinyl)-2,3-dihydro-1H-indol-6-yl)-3,4-dimethyl-1-pentyn-3-ol (266): Using the procedure described for the preparation of 249, 95 mg (59%) of 266 was obtained as a yellow solid from 145 mg (0.5 mmol) of A.95 and 224 mg (2.0 mmol) of 3,4-dimethyl-1-pentyn-3-ol C.45. ¹H NMR (500 MHz, methanol-d₄) δ 8.48 (1H, s), 8.08 (1H, d, J=7.0 Hz), 7.22 (1H, d, J=9.5 Hz), 7.04 (1H, d, J=9.5 Hz), 6.38 (2H, br s), 6.13 (1H, d, J=7.0 Hz), 5.26 (1H, s), 4.06 (2H, t, J=10.5 Hz), 3.40 (2H, t, J=10.5 Hz), 1.90 (1H, q, J=8.4 Hz), 1.50 (3H, s), 1.11 (3H, d, J=8.4 Hz), 1.08 (3H, d, J=8.4 Hz) ppm; LCMS-ESI (POS), M/Z, M+1: Found 323.3, Calculated 323.3.

### Example 267

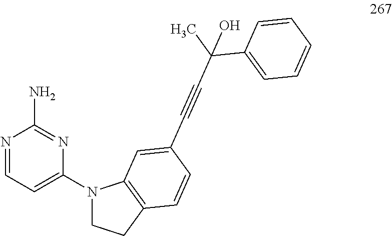

### 4-(1-(2-Amino-4-pyrimidinyl)-2,3-dihydro-1H-indol-6-yl)-2-phenyl-3-butyn-2-ol (267)

Using the procedure described for the preparation of 249, 125 mg (71%) of 267 was obtained as a light yellow solid from 145 mg (0.5 mmol) of A.95 and 292 mg (2.0 mmol) of 2-phenyl-3-butyn-2-ol C.50. ¹H NMR (500 MHz, methanol-d₄) δ 8.45 (1H, s), 7.88 (3H, m), 7.47 (3H, m), 7.17 (2H, m), 5.98 (1H, br s), 5.23 (2H, br s), 3.97 (2H, m), 3.22 (2H, m), 1.99 (3H, s) ppm; LCMS-ESI (POS), M/Z, M+1: Found 357.2, Calculated 357.2.

### Example 268

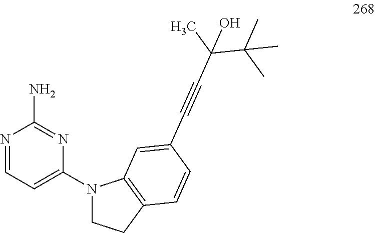

1-(1-(2-Amino-4-pyrimidinyl)-2,3-dihydro-1H-indol-6-yl)-3,4,4-trimethyl-1-pentyn-3-ol (268): Using the procedure described for the preparation of 249, 97 mg (58%) of 268 was obtained as light yellow solid from 145 mg (0.5 mmol) of A.95 and 252 mg (2.0 mmol) of 3,4,4-trimethyl-1-pentyn-3-ol C.46. ¹H NMR (500 MHz, methanol-d₄) δ 8.39 (1H, s), 7.62 (1H, m), 7.18 (1H, d, J=7.5 Hz), 6.94 (1H, d, J=7.5 Hz), 6.25 (2H, br s), 6.15 (1H, br s), 5.12 (1H, s), 3.97 (2H, t, J=8.8 Hz), 3.16 (2H, t, J=8.8 Hz), 1.43 (3H, s), 1.05 (9H, s) ppm; LCMS-ESI (POS), M/Z, M+1: Found 337.3, Calculated 337.2.

### Example 269

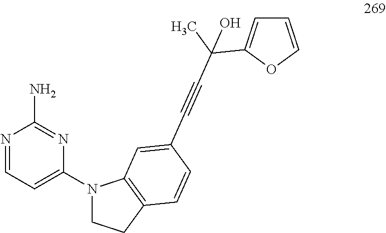

4-(1-(2-Amino-4-pyrimidinyl)-2,3-dihydro-1H-indol-6-yl)-2-(2-furanyl)-3-butyn-2-ol (269): Using the procedure described for the preparation of 249, 85 mg (49%) of 269 was obtained as light yellow solid from 145 mg (0.5 mmol) of A.95 and 272 mg (2.0 mmol) of 2-(2-furanyl)-3-butyn-2-ol C.54. ¹H NMR (500 MHz, methanol-d₄) δ 8.53 (1H, s), 8.20 (1H, br s), 7.72 (1H, d, J=1.7 Hz), 7.29 (1H, d, J=7.6 Hz), 7.08 (1H, d, J=7.6 Hz), 6.54-6.52 (2H, m), 6.42 (1H, s), 6.27 (1H, s), 6.15 (1H, d, J=5.0 Hz), 4.07 (2H, t, J=8.5 Hz), 3.40 (1H, s), 3.26 (1H, t, J=8.5 Hz), 1.89 (3H, s) ppm; LCMS-ESI (POS), M/Z, M+1: Found 347.2, Calculated 347.1.

### Example 270

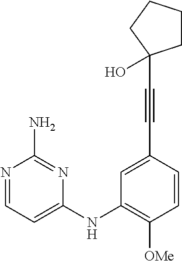

### 1-((3-((2-amino-4-pyrimidinyl)amino)-4-(methyloxy)phenyl)ethynyl)cyclopentanol (270)

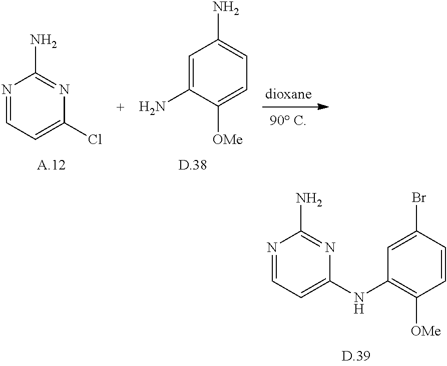

N˜4˜(5-Bromo-2-(methyloxy)phenyl)-2,4-pyrimidinediamine (D.39): Using the procedure described for the preparation of A.95, 286 mg (97%) of D.39 was obtained as yellow solid from 166 mg (1.0 mmol) of A.12 and 202 mg (1.0 mmol) of 5-bromo-2-methoxyaniline D.38. ¹H NMR (500 MHz, methanol-d₄) δ 610.50 (1H, s), 8.30 (2H, br s), 8.09 (1H, d, J=7.0 Hz), 7.50 (2H, m), 7.21 (1H, d, J=7.0 Hz), 6.50 (1H, br s), 3.94 (3H, s) ppm; LCMS-ESI (POS), M/Z, M+1: Found 295.1, Calculated 295.1.

### 1-((3-((2-Amino-4-pyrimidinyl)amino)-4-(methyloxy)phenyl)ethynyl)cyclopentanol (270)

Using the procedure described for the preparation of 249, 63 mg (39%) of 270 was obtained as light yellow solid from 147 mg (0.5 mmol) of D.39 and 220 mg (2.0 mmol) of 1-ethynylcyclopentanol C.36. ¹H NMR (500 MHz, methanol-d₄) δ 8.22 (1H, s), 8.00 (1H, d, J=5.8 Hz), 7.20 (1H, d, J=8.4 Hz), 7.08 (1H, s), 6.89 (1H, d, J=8.4 Hz), 6.17 (1H, d, J=5.8 Hz), 5.31 (2H, br s), 3.96 (3H, s), 2.20-2.15 (4H, m), 2.00-1.87 (4H, m) ppm; LCMS-ESI (POS), M/Z, M+1: Found 325.2, Calculated 325.2.

### Example 271

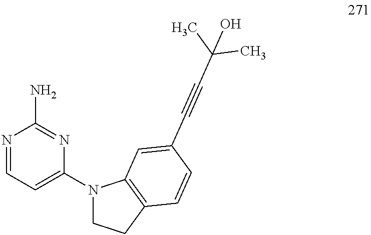

### 4-(1-(2-Amino-4-pyrimidinyl)-2,3-dihydro-1H-indol-6-yl)-2-methyl-3-butyn-2-ol (271)

Using the procedure described for the preparation of 249, 52 mg (35%) of 271 was obtained as light yellow solid from 145 mg (0.5 mmol) of A.95 and 168 mg (2.0 mmol) of 2-methyl-3-butyn-2-ol C.35. ¹H NMR (500 MHz, methanol-d₄) δ 9.00 (1H, s), 8.07 (1H, d, J=8.4 Hz), 7.25 (1H, d, J=8.6 Hz), 7.03 (1H, d. J=8.4 Hz), 6.45 (2H, br s), 6.12 (1H, d, J=8.6 Hz), 5.47 (1H, s), 4.05 (2H, t, J=7 Hz), 2.25 (2H, t, J=7 Hz), 1.58 (6H, s) ppm; LCMS-ESI (POS), M/Z, M+1: Found 295.2, Calculated 295.1.

### Example 272

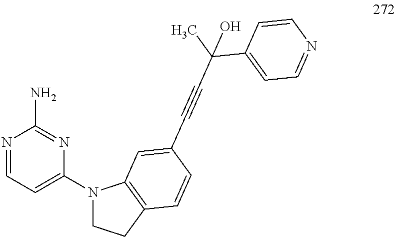

4-(1-(2-Amino-4-pyrimidinyl)-2,3-dihydro-1H-indol-6-yl)-2-(4-pyridinyl)-3-butyn-2-ol (272): Using the procedure described for the preparation of 249, 24 mg (13%) of 272 was obtained as white solid from 145 mg (0.5 mmol) of A.95 and 220 mg (1.5 mmol) of 2-(pyridin-4-yl)but-3-yn-2-ol C.47. LCMS-ESI (POS), M/Z, M+1: Found 358.1, Calculated 358.2.

### Example 273

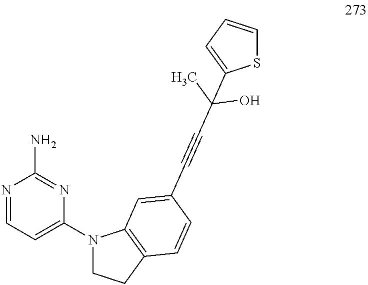

### 4-(1-(2-Amino-4-pyrimidinyl)-2,3-dihydro-1H-indol-6-yl)-2-(2-thienyl)-3-butyn-2-ol (273)

Using the procedure described for the preparation of 249, 87 mg (48%) of 273 was obtained as off-white solid from 145 mg (0.5 mmol) of A.95 and 228 mg (1.5 mmol) of 2-(thiophen-2-yl)but-3-yn-2-ol C.42 . ¹H NMR (500 MHz, CDCl₃) δ 8.46 (1H, s), 8.00 (1H, d, J=5.8 Hz), 7.43 (1H, d, J=5.0 Hz), 7.22-7.20 (2H, m), 7.00-6.99 (2H, m), 6.46 (1H, s), 6.33 (2H, br s), 6.05 (1H, d, J=5.8 Hz), 4.00 (2H, t, J=8.6 Hz), 3.18 (2H, t, J=8.6 Hz), 1.86 (3H, s) ppm; LCMS-ESI (POS), M/Z, M+1: Found 363.2, Calculated 363.1.

### Example 274

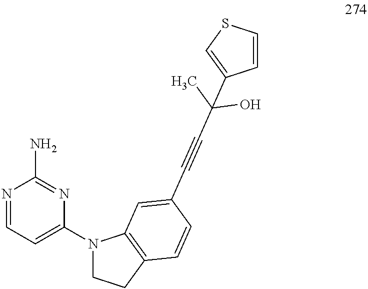

4-(1-(2-Amino-4-pyrimidinyl)-2,3-dihydro-1H-indol-6-yl)-2-(3-thienyl)-3-butyn-2-ol (274): Using the procedure described for the preparation of 249, 120 mg (66%) of 274 was obtained as a yellow solid from 145 mg (0.5 mmol) of A.95 and 304 mg (2 mmol) of 2-(thiophen-3-yl)but-3-yn-2-ol C.59. ¹H NMR (500 MHz DMSO-d6) δ 8.46 (1H, s), 8.00 (1H, d, J=5.7 Hz), 7.52-7.49 (2H, m), 7.24 (1H, d, J=5.0 Hz), 7.20 (1H, d, J=7.6 Hz), 7.00 (1H, d, J=7.6 Hz), 6.35 (2H, br s), 6.05 (1H, s), 6.01 (1H, d, J=4.7 Hz), 3.98 (2H, t, J=8.6 Hz), 3.18 (2H, t, J=8.6 Hz), 1.74 (3H, s) ppm; LCMS-ESI (POS), M/Z, M+1: Found 363.2, Calculated 363.1.

### Example 275

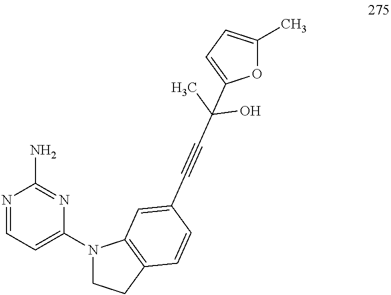

4-(1-(2-Amino-4-pyrimidinyl)-2,3-dihydro-1H-indol-6-yl)-2-(5-methyl-2-furanyl)-3-Butyn-2-ol (275): Using the procedure described for the preparation of 249, 94 mg (52%) of 275 was obtained as a yellow solid from 145 mg (0.5 mmol) of A.95 and 272 mg (2 mmol) of 2-(5-methylfuran-2-yl)but-3-yn-2-ol C.55. LCMS-ESI (POS), M/Z, M+1: Found 361.2, Calculated 361.2.

### Example 276

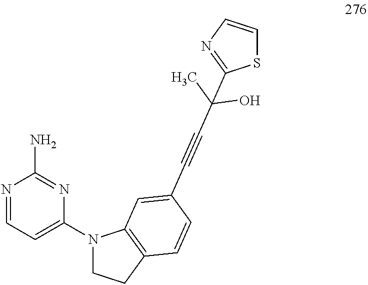

4-(1-(2-Amino-4-pyrimidinyl)-2,3-dihydro-1H-indol-6-yl)-2-(1,3-thiazol-2-yl)-3-butyn-2-ol (276): Using the procedure described for the preparation of 249, 72 mg (40%) of 276 was obtained as light a yellow solid from 145 mg (0.5 mmol) of A.95 and 306 mg (2 mmol) of 2-(thiazol-2-yl)but-3-yn-2-ol C.4. ¹H NMR (400 MHz, DMSO-d6) δ 8.52 (1H, s), 8.08 (1H, d, J=5.8 Hz), 7.86 (1H, m), 7.77 (1H, m), 7.26 (1H, d, J=7.6 Hz), 7.06-7.03 (2H, m), 6.40 (2H, br s), 6.13 (1H, d, J=5.8 Hz), 4.07 (2H, t, J=8.5 Hz), 3.26 (2H, t, J=8.5 Hz), 2.00 (3H, s) ppm; LCMS-ESI (POS), M/Z, M+1: Found 364.2, Calculated 364.1.

### Example 277

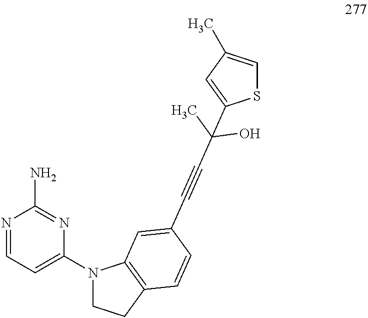

4-(1-(2-amino-4-pyrimidinyl)-2,3-dihydro-1H-indol-6-yl)-2-(4-methyl-2-thienyl)-3-butyn-2-ol (277): Using the procedure described for the preparation of 249, 88 mg (47%) of 277 was obtained as a yellow solid from 145 mg (0.5 mmol) of A.95 and 332 mg (2 mmol) of 2-(4-methylthiophen-2-yl)but-3-yn-2-ol C.60. ¹H NMR (400 MHz, DMSO-d6) δ 8.54 (1H, s), 8.08 (1H, d, J=5.7 Hz), 7.29 (1H, d, J=7.6 Hz), 7.10-7.07 (2H, m), 6.45 (1H, s), 6.40 (2H, br s), 6.14 (1H, d, J=5.7 Hz), 4.07 (2H, t, J=8.5 Hz), 3.27 (2H, t, J=8.5 Hz), 2.28 (3H, s), 1.92 (3H, s) ppm; LCMS-ESI (POS), M/Z, M+1: Found 377.2, Calculated 377.1.

### Example 278

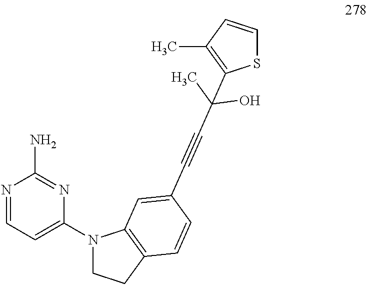

4-(1-(2-Amino-4-pyrimidinyl)-2,3-dihydro-1H-indol-6-yl)-2-(3-methyl-2-thienyl)-3-butyn-2-ol (278): Using the procedure described for the preparation of 249, 115 mg (61%) of 278 was obtained as off-white solid from 145 mg (0.5 mmol) of A.95 and 332 mg (2 mmol) of 2-(3-methylthiophen-2-yl)but-3-yn-2-ol C.61. ¹H NMR (400 MHz, DMSO-d6) δ 8.53 (1H, s), 8.09 (1H, d, J=5.7 Hz), 7.32 (1H, d, J=5.0 Hz), 7.31 (1H, d, J=7.6 Hz), 7.08 (1H, d, J=7.6 Hz), 6.93 (1H, d, J=5.0 Hz), 6.54 (1H, s), 6.35 (2H, br s), ppm; LCMS-ESI (POS), M/Z, M+1: Found 377.2, Calculated 377.1.

### Example 279

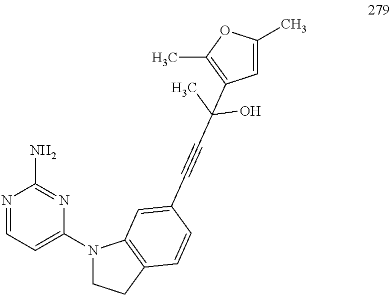

4-(1-(2-Amino-4-pyrimidinyl)-2,3-dihydro-1H-indol-6-yl)-2-(2,5-dimethyl-3-furanyl)-3-Butyn-2-ol (279): Using the procedure described for the preparation of 249, 98 mg (50%) of 279 was obtained as a yellow solid from 145 mg (0.5 mmol) of A.95 and 328 mg (2 mmol) of 2-(2,5-dimethylfuran-3-yl)but-3-yn-2-ol C.56. LCMS-ESI (POS), M/Z, M+1: Found 375.2, Calculated 375.2.

### Example 280

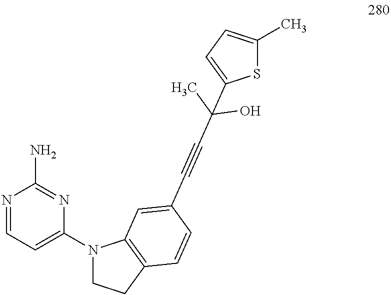

4-(1-(2-Amino-4-pyrimidinyl)-2,3-dihydro-1H-indol-6-yl)-2-(5-methyl-2-thienyl)-3-butyn-2-ol (280): Using the procedure described for the preparation of 249, 97 mg (52%) of 280 was obtained as light yellow solid from 145 mg (0.5 mmol) of A.95 and 328 mg (2 mmol) of 2-(5-methylthiophen-2-yl)but-3-yn-2-ol C.62. ¹H NMR (400 MHz, CDCl₃) δ 8.51 (1H, s), 8.04 (1H, d, J=6.0 Hz), 7.36 (1H, s), 7.20 (1H, d, J=6.0 Hz), 7.18 (1H, d, J=6.0 Hz), 7.14 (1H, d, J=3.5 Hz), 6.71 (1H, d, J=3.5 Hz), 6.08 (1H, d, J=6.0 Hz), 5.22 (2H, s), 4.07 (2H, t, J=8.4 Hz), 3.28 (2H, t, J=8.4 Hz), 2.57 (3H, s), 2.07 (3H, s) ppm; LCMS-ESI (POS), M/Z, M+1: Found 377.2, Calculated 377.1.

### Example 281

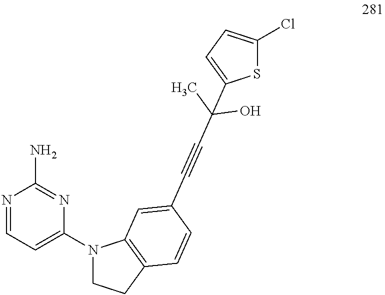

4-(1-(2-Amino-4-pyrimidinyl)-2,3-dihydro-1H-indol-6-yl)-2-(5-chloro-2-thienyl)-3-butyn-2-ol (281): Using the procedure described for the preparation of 249, 105 mg (53%) of 281 was obtained as a light yellow solid from 145 mg (0.5 mmol) of A.95 and 372 mg (2.0 mmol) of 2-(5-chlorothiophen-2-yl)but-3-yn-2-ol C.63. ¹H NMR (400 MHz, CDCl₃) δ 8.51 (1H, s), 8.00 (1H, d, J=6.0 Hz), 7.36 (1H, s), 7.21 (1H, d, J=6.0 Hz), 7.19 (1H, d, J=6.0 Hz), 7.12 (1H, d, J=3.8 Hz), 6.86 (1H, d, J=3.8 Hz), 6.07 (1H, d, J=6.0 Hz), 5.30 (2H, s), 4.07 (2H, t, J=8.5 Hz), 3.29 (2H, t, J=8.5 Hz), 2.05 (3H, s) ppm; LCMS-ESI (POS), M/Z, M+1: Found 397.2, Calculated 397.1.

### Example 282

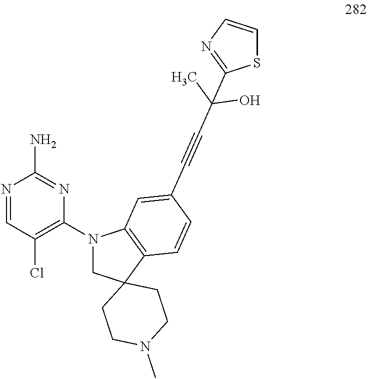

4-(1-(2-Amino-5-chloro-4-pyrimidinyl)-1′-methyl-1,2-dihydrospiro[indole-3,4′-piperidin]-6-yl)-2-(1,3-thiazol-2-yl)-3-butyn-2-ol (282): Using the procedure described for the preparation of 249, 72 mg (38%) of 282 was obtained as a light yellow solid from 204 mg (0.5 mmol) of D.37 and 306 mg (2 mmol) of 2-(thiazol-2-yl)but-3-yn-2-ol C.4. ¹H NMR (400 MHz, CDCl₃) δ 8.15 (1H, s), 7.86 (1H, d. J=3.2 Hz), 7.60 (1H, s), 7.42 (1H, d, J=3.2 Hz), 7.36 (1H, s), 7.19 (2H, s), 5.20 (2H, s), 4.19 (2H, s), 2.95 (2H, m), 2.43 (3H, s), 2.20-2.00 (7H, m), 1.79 (2H, m), 1.36 (2H, m) ppm; LCMS-ESI (POS), M/Z, M+1: Found 481.2, Calculated 481.1.

### Example 283

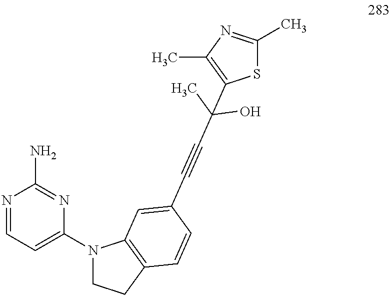

4-(1-(2-Amino-4-pyrimidinyl)-2,3-dihydro-1H-indol-6-yl)-2-(2,4-dimethyl-1,3-thiazol-5-yl)-3-butyn-2-ol (283): Using the procedure described for the preparation of 249, 59 mg (30%) of 283 was obtained as a yellow solid from 145 mg (0.5 mmol) of A.95 and 362 mg (2 mmol) of 2-(2,4-dimethylthiazol-5-yl)but-3-yn-2-ol C.69. LCMS-ESI (POS), M/Z, M+1: Found 392.2, Calculated 391.1.

### Example 284

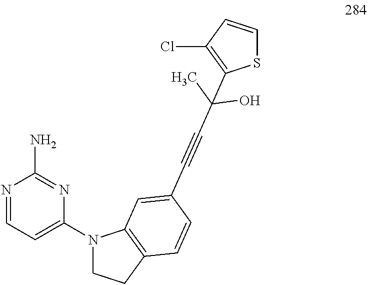

4-(1-(2-Amino-4-pyrimidinyl)-2,3-dihydro-1H-indol-6-yl)-2-(3-chloro-2-thienyl)-3-butyn-2-ol (284): Using the procedure described for the preparation of 249, 133 mg (67%) of 284 was obtained as a light yellow solid from 145 mg (0.5 mmol) of A.95 and 372 mg (2 mmol) of 2-(3-chlorothiophen-2-yl)but-3-yn-2-ol C.64. ¹H NMR (400 MHz, DMSO-d6) δ 8.44 (1H, s), 7.99 (1H, d, J=5.8 Hz), 7.49 (1H, d, J=5.3 Hz), 7.20 (1H, d, J=7.5 Hz), 7.20 (1H, d, J=5.3 Hz), 6.97 (1H, d, J=7.5 Hz), 6.81 (1H, s), 6.24 (2H, s), 6.05 (1H, d, J=5.8 Hz), 3.98 (2H, t, J=8.5 Hz), 3.17 (2H, t, J=8.5 Hz), 1.87 (3H, s) ppm; LCMS-ESI (POS), M/Z, M+1: Found 397.2, Calculated 397.1.

### Example 285

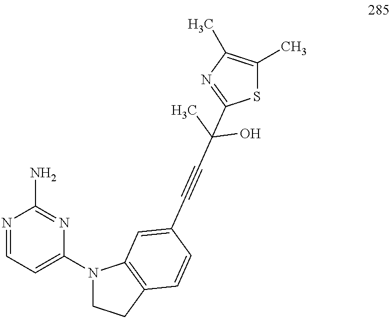

4-(1-(2-Amino-4-pyrimidinyl)-2,3-dihydro-1H-indol-6-yl)-2-(4,5-dimethyl-1,3-thiazol-2-yl)-3-Butyn-2-ol (285): Using the procedure described for the preparation of 249, 39 mg (20%) of 285 was obtained as a yellow solid from 145 mg (0.5 mmol) of A.95 and 362 mg (2 mmol) of 2-(4,5-dimethylthiazol-2-yl)but-3-yn-2-ol C.67. ¹H NMR (400 MHz, CDCl₃) δ 8.40 (1H, s), 7.90 (1H, d, J=5.8 Hz), 7.15-7.04 (2H, m), 5.95 (1H, d, J=5.7 Hz), 5.40 (2H, s), 3.86 (2H, t, J=8.5 Hz), 3.44 (1H, s), 3.15 (2H, t, J=8.5 Hz), 2.36 (6H, s), 2.02 (3H, s) ppm; LCMS-ESI (POS), M/Z, M+1: Found 392.2, Calculated 392.1.

### Example 286

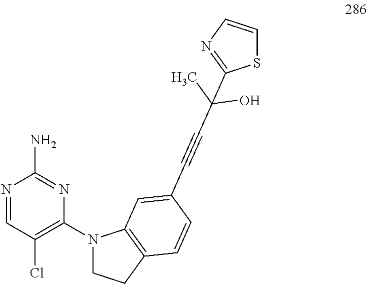

4-(1-(2-Amino-5-chloro-4-pyrimidinyl)-2,3-dihydro-1H-indol-6-yl)-2-(1,3-thiazol-2-yl)-3-Butyn-2-ol (286): Using the procedure described for the preparation of 249, 37 mg (37%) of 286 was obtained as a light yellow solid from 81 mg (0.25 mmol) of A.2 and 152 mg (1 mmol) of 2-(thiazol-2-yl)but-3-yn-2-ol C.4. LCMS-ESI (POS), M/Z, M+1: Found 398.2, Calculated 398.1.

### Example 287

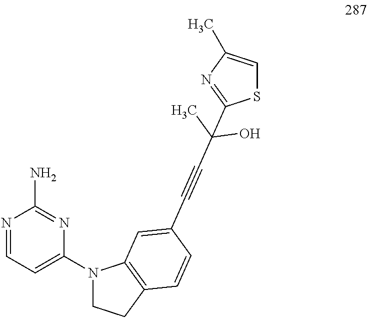

4-(1-(2-Amino-4-pyrimidinyl)-2,3-dihydro-1H-indol-6-yl)-2-(4-methyl-1,3-thiazol-2-yl)-3-Butyn-2-ol (287): Using the procedure described for the preparation of 249, 30 mg (16%) of 287 was obtained as a yellow solid from 145 mg (0.5 mmol) of A.95 and 334 mg (1 mmol) of 2-(4-methylthiazol-2-yl)but-3-yn-2-ol C.66. LCMS-ESI (POS), M/Z, M+1: Found 378.2, Calculated 378.1.

### Example 288

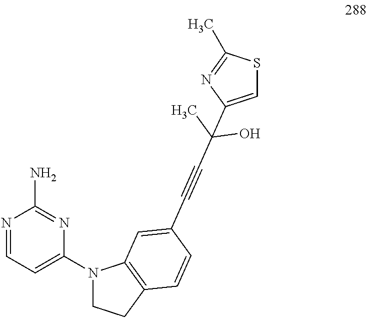

4-(1-(2-Amino-4-pyrimidinyl)-2,3-dihydro-1H-indol-6-yl)-2-(2-methyl-1,3-thiazol-4-yl)-3-butyn-2-ol (288): Using the procedure described for the preparation of 249, 32 mg (34%) of 288 was obtained as a yellow solid from 73 mg (0.25 mmol) of A.95 and 114 mg (0.68 mmol) of 2-(2-methylthiazol-4-yl)but-3-yn-2-ol C.68. ¹H NMR (400 MHz, CDCl₃) δ 8.42 (1H, s), 8.03 (1H, d, J=6.0 Hz), 7.13 (1H, d, J=7.5 Hz), 7.09 (1H, d, J=7.5 Hz), 6.05 (1H, d, J=6.0 Hz), 5.16 (2H, s), 4.03 (2H, t, J=8.5 Hz), 3.22 (2H, t, J=8.5 Hz), 2.76 (3H, s), 2.00 (3H, s) ppm; LCMS-ESI (POS), M/Z, M+1: Found 378.2, Calculated 378.1.

### Example 289

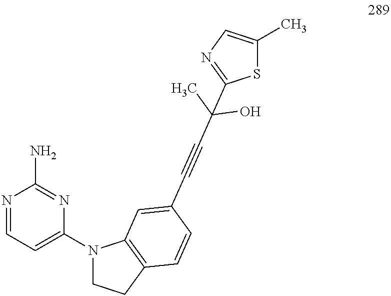

4-(1-(2-Amino-4-pyrimidinyl)-2,3-dihydro-1H-indol-6-yl)-2-(5-methyl-1,3-thiazol-2-yl)-3-Butyn-2-ol (289): Using the procedure described for the preparation of 249, 50 mg (26%) of 289 was obtained as a light yellow solid from 145 mg (0.5 mmol) of A.95 and 334 mg (2.0 mmol) of 2-(5-methylthiazol-2-yl)but-3-yn-2-ol C.65. ¹H NMR (400 MHz, CDCl₃) δ 8.42 (1H, s), 8.01 (1H, d, J=5.6 Hz), 7.43 (1H, s), 7.11 (2H, m), 6.01 (1H, d, J=5.6 Hz), 3.99 (2H, t, J=8.5 Hz), 3.20 (2H, t, J=8.5 Hz), 2.49 (3H, s), 2.05 (3H, s) ppm; LCMS-ESI (POS), M/Z, M+1: Found 378.2, Calculated 378.1.

### Example 290

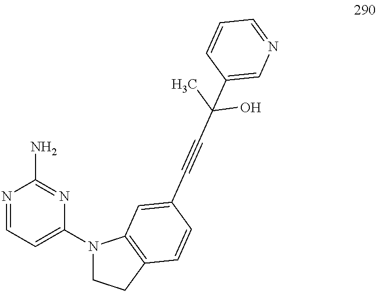

4-(1-(2-Amino-4-pyrimidinyl)-2,3-dihydro-1H-indol-6-yl)-2-(3-pyridinyl)-3-butyn-2-ol (290): Using the procedure described for the preparation of 249, 110 mg (59%) of 290 was obtained as a light yellow solid from 145 mg (0.5 mmol) of A.95 and 294 mg (2.0 mmol) of 2-(pyridin-3-yl)but-3-yn-2-ol C.48. ¹H NMR (400 MHz, CDCl₃) δ 9.07 (1H, s), 8.56 (1H, d, J=5.8 Hz), 8.44 (1H, s), 8.07 (1H, m), 7.90 (1H, m), 7.33-7.26 (2H, m), 7.16-7.10 (2H, m), 5.97 (1H, d, J=5.8 Hz), 5.22 (2H, s), 3.99 (2H, t, J=8.5 Hz), 3.21 (2H, t, J=8.5 Hz), 1.92 (3H, s) ppm; LCMS-ESI (POS), M/Z, M+1: Found 358.3, Calculated 358.2.

### Example 291

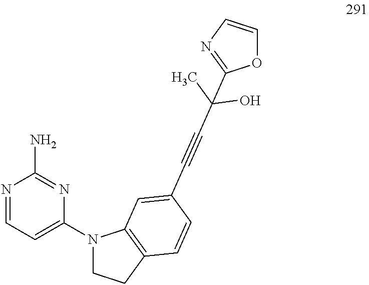

4-(1-(2-amino-4-pyrimidinyl)-2,3-dihydro-1H-indol-6-yl)-2-(1,3-oxazol-2-yl)-3-butyn-2-ol (291): Using the procedure described for the preparation of 249, 38 mg (44%) of 291 was obtained as a yellow solid from 73 mg (0.25 mmol) of A.95 and 68 mg (0.5 mmol) of 2-(oxazol-2-yl)but-3-yn-2-ol C.57. LCMS-ESI (POS), M/Z, M+1: Found 348.2, Calculated 348.1.

### Example 292

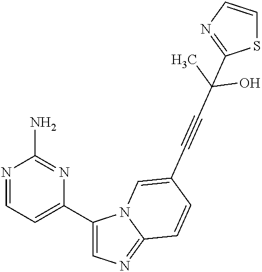

### 4-(3-(2-Amino-4-pyrimidinyl)imidazo[1,2-a]pyridin-6-yl)-2-(1,3-thiazol-2-yl)-3-butyn-2-ol (292)

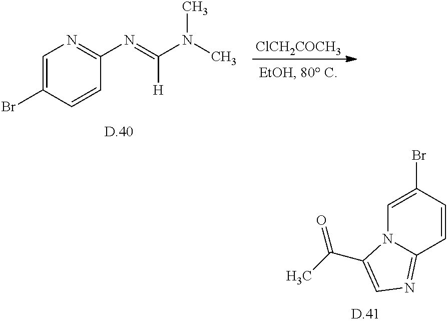

1-(6-BromoH-imidazo[1,2-a]pyridin-3-yl)ethanone (D.41): A mixture of (E)-N′-(5-bromopyridin-2-yl)-N,N-dimethylformamidine D.40 (3.42 g, 15 mmol) and chloroacetone (1.39 g, 15 mmol) in ethanol (10 mL) was stirred at 80° C. for 5 hr. The reaction mixture was concentrated and the residue was purified by flash chromatography to give the titled compound D.41 as light yellow solid (1.71 g, 48%) LCMS-ESI (POS), M/Z, M+1: Found 239.1, Calculated 239.0.

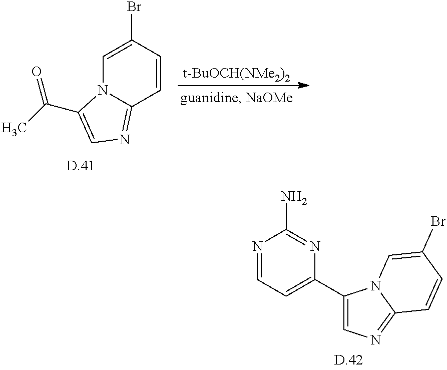

4-(6-BromoH-imidazo[1,2-a]pyridin-3-yl)pyrimidin-2-amine (D.42): A mixture of D.41 (1.2 g, 5.0 mmol) and t-butoxy-bis(dimethylamino)methane (1.31 g, 7.5 mmol, 1.55 mL) was heated at 100° C. for 2 hr. To the reaction mixture was added guanidine hydrochloride (1.91 g, 20 mmol), 30% sodium methoxide (2.8 mL, 15 mmol), and 1-propanol (10 mL). The mixture thus obtained was stirred at 85° C. for 15 hr. The reaction mixture was cooled to room temperature and the precipitate was collected to give the titled compound (D.42) as white solid (1.37 g, 95%). ¹H NMR (400 MHz, DMSO-d6) δ 10.23 (1H, d, J=2Hz), 8.53 (1H, s), 8.25 (1H, d, J=5.3 Hz), 7.72 (1H, d, J=9.5 Hz), 7.58 (1H, dd, J=9.5 Hz, J=2Hz), 7.15 (1H, d, J=5.3 Hz), 6.92 (2H, br s) ppm; LCMS-ESI (POS), M/Z, M+1: Found 290.0, Calculated 290.0.

4-(3-(2-Amino-4-pyrimidinyl)imidazo[1,2-a]pyridin-6-yl)-2-(1,3-thiazol-2-yl)-3-butyn-2-ol (292): Using the procedure described for the preparation of 249, 26 mg (81%) of 292 was obtained as a light yellow solid from 26 mg (0.089 mmol) of D.42 and 54 mg (0.35 mmol) of 2-(thiazol-2-yl)but-3-yn-2-ol C.4. LCMS-ESI (POS), M/Z, M+1: Found 363.2, Calculated 363.1.

### Example 293

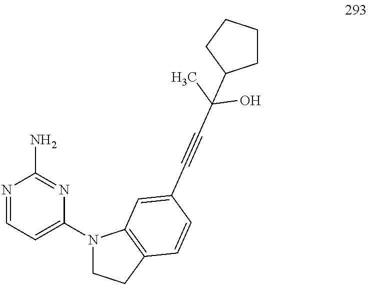

4-(1-(2-Amino-4-pyrimidinyl)-2,3-dihydro-1H-indol-6-yl)-2-cyclopentyl-3-butyn-2-ol (293): Using the procedure described for the preparation of 249, 56 mg (58%) of 293 was obtained as light yellow solid from 73 mg (0.25 mmol) of A.95 and 138 mg (1.0 mmol) of 2-cyclopentylbut-3-yn-2-ol C.34. ¹H NMR (400 MHz, CDCl₃) δ 8.39 (1H, s), 8.20 (1H, d, J=6.0 Hz), 7.12 (1H, d, J=7.6 Hz), 7.02 (1H, d. J=7.6 Hz), 6.05 (1H, d, J=6.0 Hz), 5.00 (2H, s), 4.02 (2H, t, J=8.5 Hz), 3.21 (2H, t, J=8.5 Hz), 2.23 (1H, m), 1.87-1.55 (8H, m), 1.60 (3H, s) ppm; LCMS-ESI (POS), M/Z, M+1: Found 349.3, Calculated 349.2.

### Example 294

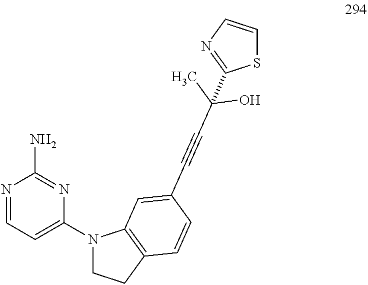

(2R)-4-(1-(2-amino-4-pyrimidinyl)-2,3-dihydro-1H-indol-6-yl)-2-(1,3-thiazol-2-yl)-3-Butyn-2-ol (294): Using the procedure described for the preparation of 249, 20 mg (28%) of 294 was obtained as a light yellow solid from 58 mg (0.2 mmol) of A.95 and 64 mg (0.418 mmol) of (R)-2-(thiazol-2-yl)but-3-yn-2-ol C.6. ¹H NMR (400 MHz, CDCl₃) δ 8.43 (1H, s), 8.02 (1H, d, J=6.0 Hz), 7.80 (1H, d, J=3.2 Hz), 7.35 (1H, d, J=3.2 Hz), 7.13 (1H, d, J=7.7 Hz), 7.10 (1H, d, J=7.7 Hz), 6.02 (1H, d, J=6.0 Hz), 5.20 (2H, br s), 4.00 (2H, t, J=8.5 Hz), 3.21 (2H, t, J=8.5 Hz), 2.07 (3H, s) ppm; LCMS-ESI (POS), M/Z, M+1: Found 364.2, Calculated 364.1.

### Example 295

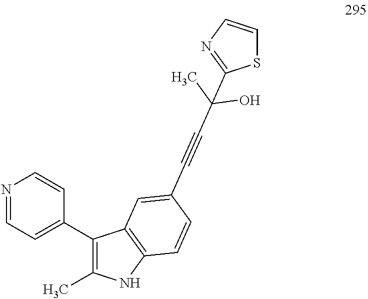

4-(2-Methyl-3-(4-pyridinyl)-1H-indol-5-yl)-2-(1,3-thiazol-2-yl)-3-butyn-2-ol (295): Using the procedure described for the preparation of 249, 96 mg (53%) of 295 was obtained as light yellow solid from 144 mg (0.5 mmol) of D.43 and 306 mg (2.0 mmol) of 2-(thiazol-2-yl)but-3-yn-2-ol C.4. ¹H NMR (400 MHz, methanol-d₄) δ 7.07 (1H, d, J=5 Hz), 6.32 (1H, s), 6.28 (1H, s), 6.09-6.06 (3H, m), 5.84 (1H, d, J=8.4 Hz), 5.76 (1H, d, J=8.4 Hz), 1.83 (6H, s) ppm; LCMS-ESI (POS), M/Z, M+1: Found 360.2, Calculated 360.1.

### Example 296

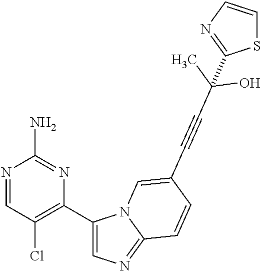

### (2R)-4-(3-(2-amino-5-chloro-4-pyrimidinyl)imidazo[1,2-a]pyridin-6-yl)-2-(1,3-thiazol-2-yl)-3-butyn-2-ol (296)

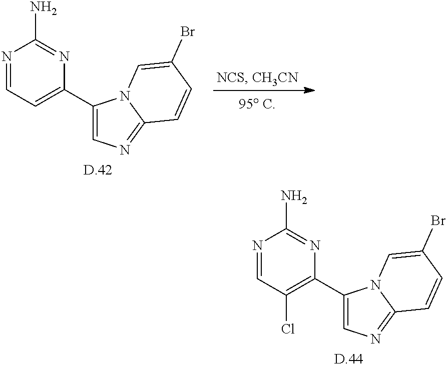

4-(6-Bromoimidazo[1,2-a]pyridin-3-yl)-5-chloro-2-pyrimidinamine (D.44): To a solution of D.42 (245 mg, 0.845 mmol) in acetonitrile (5 mL) was added N-chlorosuccinimide (133 mg, 1.0 mmol). The reaction mixture thus obtained was stirred at 95° C. for 18 hr. The reaction mixture was concentrated and the residue was purified by flash chromatography on silica gel eluting with 1.25% methanol in dichloromethane to give compound D.44 as a light yellow solid (250 mg, 91%) ¹H NMR (400 MHz, DMSO-d6) δ 9.99 (1H, s), 8.63 (1H, s), 8.43 (1H, s), 7.77 (1H, d, J=9.4 Hz), 7.64 (1H, d, J=9.4 Hz), 7.16 (2H, br s) ppm; LCMS-ESI (POS), M/Z, M+1: Found 324.0, Calculated 324.0.

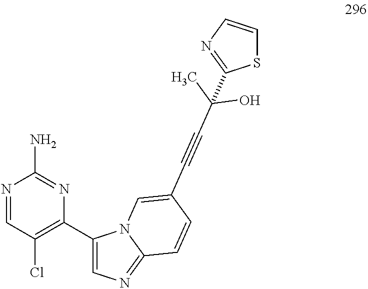

(2R)-4-(3-(2-amino-5-chloro-4-pyrimidinyl)imidazo[1,2-a]pyridin-6-yl)-2-(1,3-thiazol-2-yl)-3-butyn-2-ol (296): Using the procedure described for the preparation of 249, 43 mg (43%) of 296 was obtained as a light yellow solid from 81 mg (0.25 mmol) of D.44 and 306 mg (2.0 mmol) of (R)-2-(thiazol-2-yl)but-3-yn-2-ol C.6. ¹H NMR (400 MHz, CDCl₃) δ 9.75 (1H, s), 8.73 (1H, s), 8.31 (1H, s), 7.80 (1H, d, J=5.0 Hz), 7.65 (1H, d, J=5.0 Hz), 7.38-7.33 (2H, m), 5.32 (2H, br s), 2.09 (3H, s) ppm; LCMS-ESI (POS), M/Z, M+1: Found 397.1, Calculated 397.1.

### Example 297

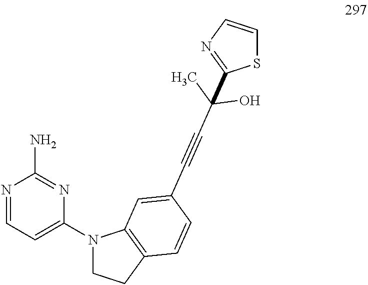

(2S)-4-(1-(2-amino-4-pyrimidinyl)-2,3-dihydro-1H-indol-6-yl)-2-(1,3-thiazol-2-yl)-3-butyn-2-ol (297): Using the procedure described for the preparation of 249, 18 mg (25%) of 297 was obtained as a light yellow solid from 58 mg (0.2 mmol) of A.95 and 92 mg (0.6 mmol) of (S)-2-(thiazol-2-yl)but-3-yn-2-ol C.5. LCMS-ESI (POS), M/Z, M+1: Found 364.2, Calculated 364.1.

### Example 298

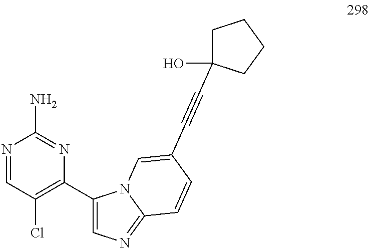

1-((3-(2-amino-5-chloro-4-pyrimidinyl)imidazo[1,2-a]pyridin-6-yl)ethynyl)-cyclopentanol (298): Using the procedure described for the preparation of 249, 60 mg (68%) of 298 was obtained as light yellow solid from 81 mg (0.25 mmol) of D.44 and 220 mg (2.0 mmol) of 1-ethynyl-cyclopentanol C.36. ¹H NMR (400 MHz, CDCl₃) δ 9.77 (1H, s), 8.77 (1H, d, J=1.6 Hz)), 8.38 (1H, s), 7.70 (1H, d, J=9.2 Hz), 7.38 (1H, dd, J=9.2 Hz, J=1.6 Hz), 5.20 (2H, s), 2.21-1.66 (8H, m) ppm; LCMS-ESI (POS), M/Z, M+1: Found 354.2, Calculated 354.1.

### Example 299

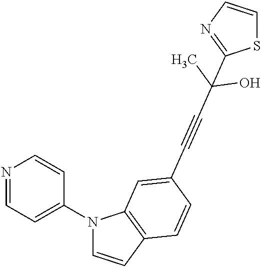

### 4-(1-(4-pyridinyl)-1H-indol-6-yl)-2-(1,3-thiazol-2-yl)-3-butyn-2-ol

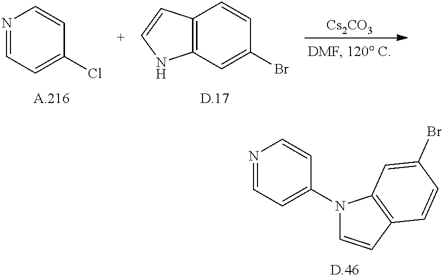

6-Bromo-1-(pyridin-4-yl)-1H-indole (D.46): A mixture of 4-chloropyridine hydrochloride (A.216) (1.5 g, 10 mmol), 6-bromoindole D.17 (1.96 g, 10 mmol), and cesium carbonate (9.8 g, 30 mmol) in DMF (20 mL) was stirred at 120° C. for 24 hr and cooled to room temperature. After filtration, the filtrate was concentrated and the residue was dissolved in ethyl acetate and washed with 5% sodium bicarbonate, brine then dried. The solvent was evaporated and the residue was purified by chromatography on silica gel eluting with 25-100% ethyl acetate in hexane to give compound D.46 as a light yellow solid (896 mg, 34%). ¹H NMR (400 MHz, CDCl₃) δ 8.77 (2H, d, J=5 Hz), 7.86 (1H, s), 7.56 (1H, d, J=7.4 Hz), 7.46 (2H, d, J=5 Hz), 7.38-7.34 (2H, m), 6.75 (1H, d, J=3.4 Hz) ppm; LCMS-ESI (POS), M/Z, M+1: Found 273.0, Calculated 273.0.

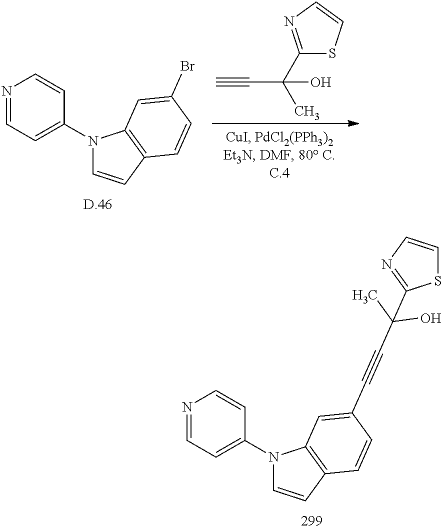

4-(1-(4-Pyridinyl)-1H-indol-6-yl)-2-(1,3-thiazol-2-yl)-3-butyn-2-ol (299): Using the procedure described for the preparation of 249, 41 mg (24%) of 299 was obtained as a light yellow solid from 136 mg (0.5 mmol) of D.46 and 306 mg (2.0 mmol) of 2-(thiazol-2-yl)but-3-yn-2-ol C.4. ¹H NMR (400 MHz, CDCl₃) δ 8.76 (2H, d, J=5 Hz), 7.81 (1H, s), 7.80 (1H, d, J=3.2 Hz), 7.62 (1H, d, J=8.2 Hz), 7.45 (2H, d, J=5 Hz), 7.43 (1H, d, J=3.2 Hz), 7.35 (1H, d, J=3.2 Hz), 7.32 (1H, d, J=8.2 Hz), 6.75 (1H, d, J=3.2 Hz), 2.09 (3H, s) ppm; LCMS-ESI (POS), M/Z, M+1: Found 346.2, Calculated 346.1.

### Example 300

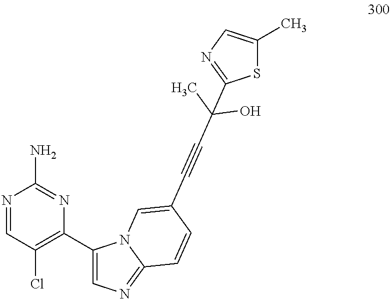

4-(3-(2-Amino-5-chloro-4-pyrimidinyl)imidazo[1,2-a]pyridin-6-yl)-2-(5-methyl-1,3-thiazol-2-yl)-3-butyn-2-ol (300): Using the procedure described for the preparation of 249, 44 mg (22%) of 300 was obtained as white solid from 162 mg (0.5 mmol) of D.44 and 250 mg (1.5 mmol) of 2-(5-methylthiazol-2-yl)but-3-yn-2-ol C.65. ¹H NMR (400 MHz, methanol-d₄) δ 10.09 (1H, s), 8.73 (1H, s), 8.34 (1H, s), 7.70 (1H, d, J=9.0 Hz), 7.76 (1H, d, J=9.0 Hz), 7.45 (1H, s), 2.51 (3H, s), 1.96 (3H, s) ppm; LCMS-ESI (POS), M/Z, M+1: Found 411.2, Calculated 411.1.

### Example 301

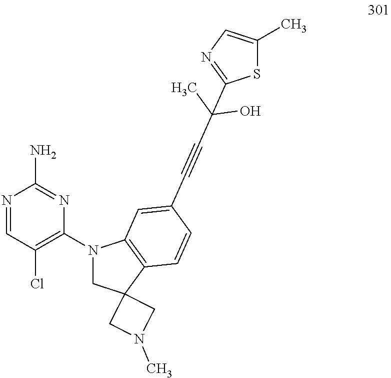

### 4-(1′-(2-Amino-5-chloro-4-pyrimidinyl)-1-methyl-1′,2′-dihydrospiro[azetidine-3,3′-indol]-6′-yl)-2-(5-methyl-1,3-thiazol-2-yl)-3-butyn-2-ol (301)

Using the procedure described for the preparation of 249, 103 mg (89%) of 301 was obtained as a white solid from 112 mg (0.25 mmol) of D.45 and 84 mg (1.5 mmol) of 2-(5-methylthiazol-2-yl)but-3-yn-2-ol C.65. ¹H NMR (400 MHz, CDCl₃) δ 8.08 (1H, s), 7.63 (1H, s), 7.48 (1H, d, J=7.7 Hz), 7.41 (1H, d, J=1.2 Hz), 7.17 (1H, dd, J=7.7 Hz, J=1.2 Hz), 5.02 (2H, br s), 3.48-3.42 (4H, m), 2.49 (3H, s), 2.42 (3H, s), 2.02 (3H, s) ppm; LCMS-ESI (POS), M/Z, M+1: Found 467.2, Calculated 467.1.

### Example 302

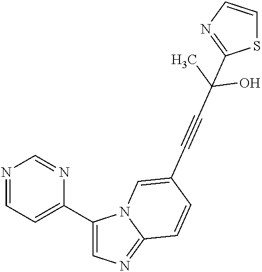

### 4-(3-(4-pyrimidinyl)imidazo[1,2-a]pyridin-6-yl)-2-(1,3-thiazol-2-yl)-3-butyn-2-ol (302)

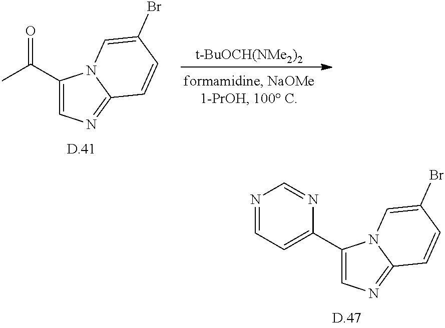

6-Bromo-3-(pyrimidin-4-yl)H-imidazo[1,2-a]pyridine (D.47): To a mixture of D.41 (119 mg, 0.5 mmol) and t-butyl-bis(dimethylamine)methane (131 mg, 0.75 mmol, 155 uL) was added dichloromethane. The mixture stirred at 100° C. to evaporate all dichloromethane. The experiment was repeated one more time then to the reaction mixture were added formamidine acetate (156 mg, 1.5 mmol), 30% sodium methoxide (186 uL, 1.0 mmol), and 1-propanol (2 mL). The mixture thus obtained was stirred at 100° C. for 20 hr. The reaction mixture was concentrated and the residue was purified by flash chromatography on silica gel eluting with 2.5% methanol in dichloromethane to give compound D.47 as a light yellow solid (45 mg, 33%) ¹H NMR (400 MHz, CDCl₃) δ 10.60 (1H, s), 9.27 (1H, s), 8.72 (1H, d, J=5.5 Hz), 8.35 (1H, s), 7.68-7.66 (2H, m), 7.50 (1H, d, J=7.8 Hz) ppm; LCMS-ESI (POS), M/Z, M+1: Found 275.0, Calculated 275.0.

4-(3-(4-pyrimidinyl)imidazo[1,2-a]pyridin-6-yl)-2-(1,3-thiazol-2-yl)-3-butyn-2-ol (302): Using the procedure described for the preparation of 249, 21 mg (48%) of 302 was obtained as a white solid from 35 mg (0.127 mmol) of D.47 and 61 mg (0.4 mmol) of 2-(thiazol-2-yl)but-3-yn-2-ol C.4. ¹H NMR (400 MHz, CDCl₃) δ 10.13 (1H, s), 9.23 (1H, s), 8.67 (1H, d, J=5.6 Hz), 8.33 (1H, s), 7.82 (1H, d, J=3.2 Hz), 7.70 (1H, d, J=9.2 Hz), 7.63 (1H, d, J=5.6 Hz), 7.42 (1H, d, J=9.2 Hz), 7.39 (1H, d, J=3.2 Hz), 5.32 (1H, br s), 2.14 (3H, s) ppm; LCMS-ESI (POS), M/Z, M+1: Found 348.2, Calculated 348.1.

### Example 303

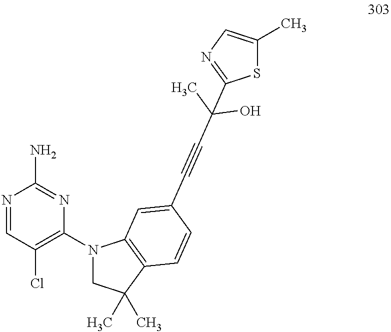

4-(1-(2-Amino-5-chloro-4-pyrimidinyl)-3,3-dimethyl-2,3-dihydro-1H-indol-6-yl)-2-(5-methyl-1,3-thiazol-2-yl)-3-butyn-2-ol (303): To a degassed solution of A.15 (100 mg, 0.25 mmol) in piperidine (1 mL) were added tetrakis(triphenylphosphine)palladium (0) (29 mg, 0.025 mmol), CuI (5 mg, 0.025 mmol), and 2-(5-methylthiazol-2-yl)but-3-yn-2-ol C.65 (84 mg, 0.5 mmol). The resulting mixture was stirred at room temperature for 20 min under argon atmosphere. The reaction mixture was concentrated and the residue was purified by flash chromatography on silica gel eluting with 30-100% ethyl acetate in hexane to give compound 303 as a light yellow solid (91 mg, 84%). ¹H NMR (400 MHz, CDCl₃) δ 8.03 (1H, s), 7.58 (1H, s), 7.40 (1H, s), 7.12 (1H, d, J=7.7 Hz), 7.07 (1H, d, J=7.7 Hz), 5.27 (2H, br s), 4.02 (2H, m), 2.46 (3H, s), 2.06 (3H, s), 1.32 (6H, s) ppm; LCMS-ESI (POS), M/Z, M+1: Found 340.2, Calculated 340.1.

### Example 304

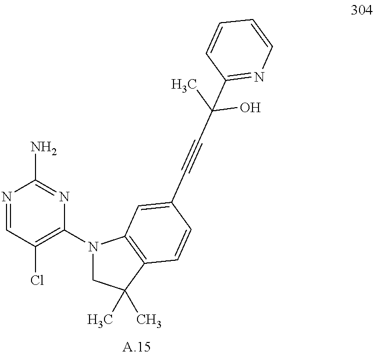

4-(1-(2-Amino-5-chloro-4-pyrimidinyl)-3,3-dimethyl-2,3-dihydro-1H-indol-6-yl)-2-(2-pyridinyl)-3-Butyn-2-ol (304): Using the procedure described for example 303, 76 mg (72%) of 304 was obtained as a yellow solid from 100 mg (0.25 mmol) of A.15 and 74 mg (0.5 mmol) of 2-(pyridin-2-yl)but-3-yn-2-ol C.49. ¹H NMR (400 MHz, CDCl₃) δ 8.62 (1H, d, J=5 Hz), 8.07 (1H, s), 7.78 (1H, t, J=7.7 Hz), 7.70 (1H, d, J=7.7 Hz), 7.54 (1H, s), 7.29 (1H, m), 7.10 (1H, d, J=7.5 Hz), 7.08 (1H, d, J=7.5 Hz), 5.00 (2H, br s), 4.02 (2H, s), 1.88 (3H, s), 1.34 (6H, s) ppm; LCMS-ESI (POS), M/Z, M+1: Found 420.2, Calculated 420.1.

### Example 305

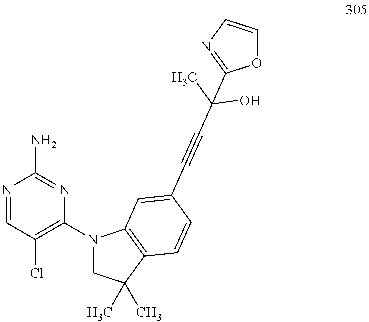

4-(1-(2-Amino-5-chloro-4-pyrimidinyl)-3,3-dimethyl-2,3-dihydro-1H-indol-6-yl)-2-(1,3-oxazol-2-yl)-3-Butyn-2-ol (305): Using the procedure described in example 303, 107 mg (100%) of compound 305 was obtained as a yellow solid from 100 mg (0.25 mmol) of A.15 and 68 mg (0.5 mmol) of 2-(oxazol-2-yl)but-3-yn-2-ol C.57. ¹H NMR (400 MHz, CDCl₃) δ 8.03 (1H, s), 7.66 (1H, s), 7.59 (1H, s), 7.13-7.07 (3H, m), 5.36 (2H, br s), 4.02 (2H, s), 2.03 (3H, s), 1.32 (6H, s) ppm; LCMS-ESI (POS), M/Z, M+1: Found 410.1, Calculated 410.1.

### Example 306

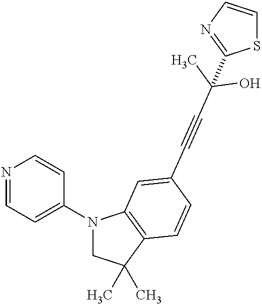

### (2R)-4-(3,3-dimethyl-1-(4-pyridinyl)-2,3-dihydro-1H-indol-6-yl)-2-(1,3-thiazol-2-yl)-3-Butyn-2-ol (306)

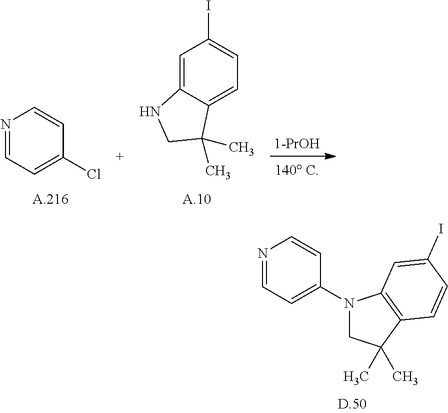

6-Iodo-3,3-dimethyl-1-(pyridin-4-yl)indoline (D.50): A mixture of 4-chloropyridine (A.216)(574 mg, 3.82 mmol) and A.10 (522 mg, 1.91 mmol) in 1-pentanol (5 mL) was stirred at 140° C. overnight. The reaction mixture was concentrated and the residue was purified by flash chromatography on silica gel eluting with 0.25% NH₄OH and 2.5% methanol in dichloromethane to give compound D.50 as a yellow solid (580 mg, 88%). ¹H NMR (400 MHz, CDCl₃) δ 8.46 (2H, d, J=6.0 Hz), 7.65 (1H, s), 7.27 (1H, d, J=7.8 Hz), 7.03 (2H, d, J=6.0 Hz), 6.91 (1H, d, J=7.8 Hz), 3.72 (2H, s), 1.36 (6H, s) ppm; LCMS-ESI (POS), M/Z, M+1: Found 351.0, Calculated 351.1.

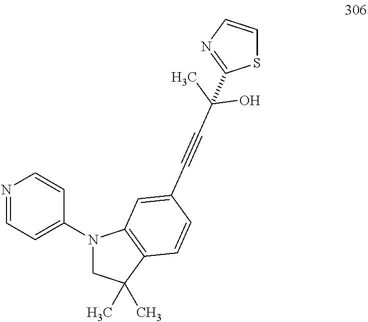

(2R)-4-(3,3-Dimethyl-1-(4-pyridinyl)-2,3-dihydro-1H-indol-6-yl)-2-(1,3-thiazol-2-yl)-3-butyn-2-ol (306): Using the procedure described in example 303, 91 mg (98%) of 306 was obtained as a yellow solid from 88 mg (0.25 mmol) of D.50 and 57 mg (0.375 mmol) of (R)-2-(thiazol-2-yl)but-3-yn-2-ol C.6 . ¹H NMR (400 MHz, CDCl₃) δ 8.42 (2H, d, J=6.0 Hz), 7.79 (1H, d, J=3.2 Hz), 7.36 (1H, s), 7.34 (1H, d, J=3.2 Hz), 7.09 (2H, m), 7.02 (2H, d, J=6.0 Hz), 3.71 (2H, s), 2.07 (3H, s), 1.38 (6H, s) ppm; LCMS-ESI (POS), M/Z, M+1: Found 376.1, Calculated 376.1.

### Example 307

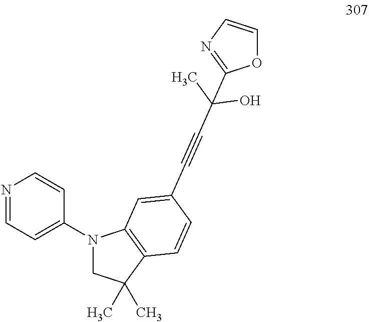

4-(3,3-Dimethyl-1-(4-pyridinyl)-2,3-dihydro-1H-indol-6-yl)-2-(1,3-oxazol-2-yl)-3-butyn-2-ol (307): Using the procedure described in example 303, 89 mg (100%) of 307 was obtained as a yellow solid from 88 mg (0.25 mmol) of D.50 and 68 mg (0.5 mmol) of (2-(oxazol-2-yl)but-3-yn-2-ol C.57. ¹H NMR (400 MHz, CDCl₃) δ 7.70 (2H, d, J=6.0 Hz), 7.38 (1H, s), 7.34 (1H, s), 7.11 (2H, m), 7.10-7.08 (4H, m), 3.71 (2H, s), 2.05 (3H, s), 1.37 (6H, s) ppm; LCMS-ESI (POS), M/Z, M+1: Found 360.1, Calculated 360.1.

### Example 308

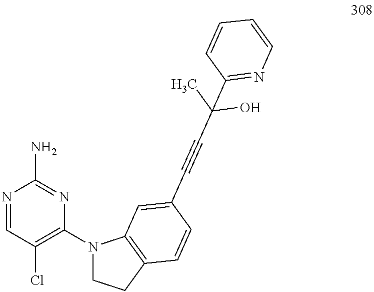

4-(1-(2-Amino-5-chloro-4-pyrimidinyl)-2,3-dihydro-1H-indol-6-yl)-2-(2-pyridinyl)-3-butyn-2-ol (308): Using the procedure described in example 303, 28 mg (78%) of 308 was obtained as a yellow solid from 35 mg (0.094 mmol) of A.3 and 29 mg (0.2 mmol) of 2-(pyridin-2-yl)but-3-yn-2-ol C.49. ¹H NMR (400 MHz, CDCl₃) δ 8.55 (1H, d, J=5.0 Hz), 8.06 (1H, s), 7.77 (1H, t, J=7.8 Hz), 7.70 (1H, d, J=7.8 Hz), 7.55 (1H, s), 7.27 (1H, m), 7.12 (1H, d, J=7.6 Hz), 7.05 (1H, d, J=7.6 Hz), 5.02 (2H, br s), 4.30 (2H, t, J=8.5 Hz), 3.13 (2H, t, J-8.5 Hz), 1.88 (3H, s) ppm; LCMS-ESI (POS), M/Z, M+1: Found 392.2, Calculated 392.1.

### Example 309

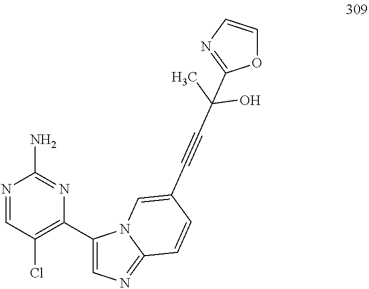

4-(3-(2-Amino-5-chloro-4-pyrimidinyl)imidazo[1,2-a]pyridin-6-yl)-2-(1,3-oxazol-2-yl)-3-Butyn-2-ol (309): Using the procedure described for the preparation of 249, 81 mg (50%) of 309 was obtained as a yellow solid from 162 mg (0.5 mmol) of D.44 and 272 mg (2.0 mmol) of 2-(oxazol-2-yl)but-3-yn-2-ol C.57. LCMS-ESI (POS), M/Z, M+1: Found 381.0, Calculated 381.0.

### Example 310

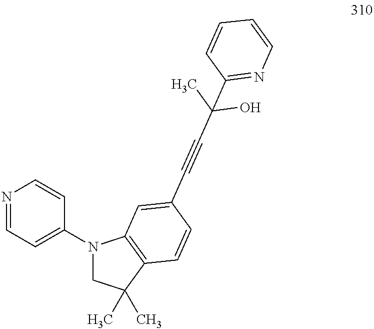

4-(3,3-Dimethyl-1-(4-pyridinyl)-2,3-dihydro-1H-indol-6-yl)-2-(2-pyridinyl)-3-butyn-2-ol (310): Using the procedure described in example 303, 83 mg (90%) of 310 was obtained as a yellow solid from 88 mg (0.25 mmol) of D.50 and 73 mg (0.5 mmol) of 2-(pyridin-2-yl)but-3-yn-2-ol C.49. ¹H NMR (400 MHz, CDCl₃) δ 8.56 (2H, d, J=6.0 Hz), 7.81 (1H, t, J=7.8 Hz), 7.70 (1H, d, J=7.8 Hz), 7.39 (1H, s), 7.29 (2H, m), 7.11-7.05 (4H, m), 3.73 (2H, s), 1.90 (3H, s), 1.37 (6H, s) ppm; LCMS-ESI (POS), M/Z, M+1: Found 370.2, Calculated 370.2.

### Example 311

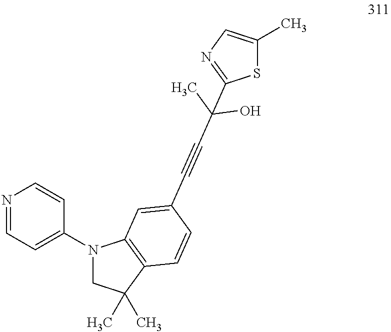

4-(3,3-Dimethyl-1-(4-pyridinyl)-2,3-dihydro-1H-indol-6-yl)-2-(5-methyl-1,3-thiazol-2-yl)-3-butyn-2-ol (311): Using the procedure described in example 303, 97 mg (100%) of 311 was obtained as yellow solid from 88 mg (0.25 mmol) of D.50 and 84 mg (0.5 mmol) of 2-(5-methylthiazol-2-yl)but-3-yn-2-ol C.65. ¹H NMR (400 MHz, CDCl₃) δ 8.44 (2H, d, J=5.6 Hz), 7.41 (1H, s), 7.38 (1H, s), 7.10 (1H, d, J=7.6 Hz), 7.07 (1H, d, J=7.6 Hz), 7.04 (2H, d, J=5.6 Hz), 3.72 (2H, s), 2.49 (3H, s), 2.03 (3H, s), 1.37 (6H, s) ppm; LCMS-ESI (POS), M/Z, M+1: Found 390.2, Calculated 390.2.

### Example 312

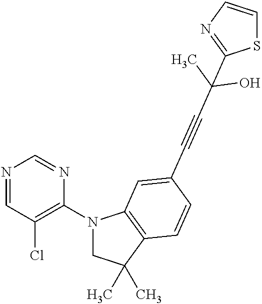

### 4-(1-(5-chloro-4-pyrimidinyl)-3,3-dimethyl-2,3-dihydro-1H-indol-6-yl)-2-(1,3-thiazol-2-yl)-3-butyn-2-ol (312)

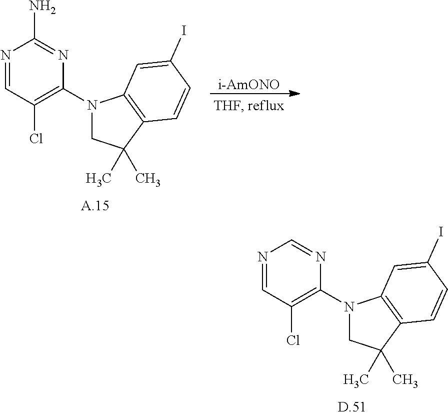

1-(5-Chloropyrimidin-4-yl)-6-iodo-3,3-dimethylindoline (D.51): To a solution of A.15 (100 mg, 0.25 mmol) in THF (5 mL) was added isoamyl nitrite (59 mg, 0.5 mmol, 67 uL) dropwise at room temperature then the mixture was stirred at 80° C. for 14 hr. The reaction mixture was concentrated and the residue was purified by flash chromatography on silica gel eluting with 10-60% ethyl acetate in hexane to give compound D.51 as a colorless oil (30 mg, 31%). LCMS-ESI (POS), M/Z, M+1: Found 386.0, Calculated 386.0.

4-(1-(5-Chloro-4-pyrimidinyl)-3,3-dimethyl-2,3-dihydro-1H-indol-6-yl)-2-(1,3-thiazol-2-yl)-3-butyn-2-ol (312): Using the procedure described in example 303, 30 mg (94%) of 312 was obtained as a yellow solid from 30 mg (0.078 mmol) of D.51 and 24 mg (0.16 mmol) of 2-(thiazol-2-yl)but-3-yn-2-ol C.4. ¹H NMR (400 MHz, CDCl₃) δ 8.69 (1H, s), 8.48 (1H, s), 8.78 (1H, d, J=2.9 Hz), 7.62 (1H, s), 7.34 (1H, d, J=2.9 Hz), 7.17 (1H, d, J=7.7 Hz), 7.11 (1H, d, J-7.7 Hz), 4.09 (2H, s), 2.05 (3H, s), 1.36 (6H, s) ppm; LCMS-ESI (POS), M/Z, M+1: Found 411.2, Calculated 411.1.

### Example 313

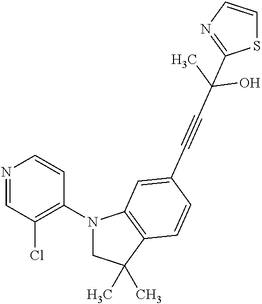

### 4-(1-(3-Chloro-4-pyridinyl)-3,3-dimethyl-2,3-dihydro-1H-indol-6-yl)-2-(1,3-thiazol-2-yl)-3-butyn-2-ol (313)

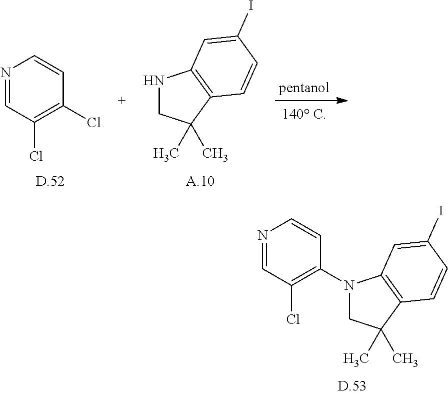

1-(3-Chloropyridin-4-yl)-6-iodo-3,3-dimethylindoline (D.53): A mixture of 3,4-dichloropyridine D.52 (740 mg, 5 mmol) and compound A.10 (1.25 g, 4.0 mmol) in 1-pentanol (5 mL) was stirred at 140° C. overnight. The reaction mixture was concentrated and the residue was purified by flash chromatography on silica gel eluting with 0.2% NH₄OH and 2% methanol in dichloromethane to give compound D.53 as a brown oil (1.02 g, 67%). ¹H NMR (400 MHz, CDCl₃) δ 8.57 (1H, s), 8.38 (1H, d, J=5.5 Hz), 7.30 (1H, d, J=5.5 Hz), 7.26 (1H, d, J=7.8 Hz), 7.09 (1H, s), 6.91 (1H, d, J=7.8 Hz) ppm; LCMS-ESI (POS), M/Z, M+1: Found 395.0, Calculated 395.0.

4-(1-(3-Chloro-4-pyridinyl)-3,3-dimethyl-2,3-dihydro-1H-indol-6-yl)-2-(1,3-thiazol-2-yl)-3-butyn-2-ol (313): Using the procedure described in example 303, 81 mg (99%) of 313 was obtained as a yellow solid from 77 mg (0.2 mmol) of D.53 and 60 mg (0.4 mmol) of 2-(thiazol-2-yl)but-3-yn-2-ol C.4. ¹H NMR (400 MHz, CDCl₃) δ 8.53 (1H, s), 8.33 (1H, d, J=5.5 Hz), 7.76 (1H, d, J=3.2 Hz), 7.32-7.28 (2H, m), 7.10 (1H, d, J=7.6 Hz), 7.06 (1H, d, J=7.6 Hz), 6.85 (1H, s), 5.13 (1H, br s), 3.79 (2H, s), 2.04 (3H, s), 1.35 (6H, s) ppm; LCMS-ESI (POS), M/Z, M+1: Found 410.2, Calculated 410.1.

### Example 314

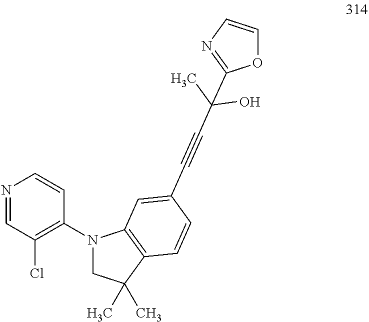

4-(1-(3-chloro-4-pyridinyl)-3,3-dimethyl-2,3-dihydro-1H-indol-6-yl)-2-(1,3-oxazol-2-yl)-3-butyn-2-ol (314): Using the procedure described in example 303, 78 mg (99%) of 314 was obtained as a light yellow solid from 77 mg (0.2 mmol) of D.53 and 54 mg (0.4 mmol) of 2-(oxazol-2-yl)but-3-yn-2-ol C.57. ¹H NMR (400 MHz, CDCl₃) δ 8.53 (1H, s), 8.33 (1H, d, J=5.3 Hz), 7.64 (1H, s), 7.27 (1H, d, J=5.3 Hz), 7.11 (1H, s), 7.09 (1H, d, J=7.6 Hz), 7.03 (1H, d, J=7.6 Hz), 6.83 (1H, s), 3.79 (2H, s), 2.01 (3H, s), 1.34 (6H, s) ppm; LCMS-ESI (POS), M/Z, M+1: Found 394.2, Calculated 394.1.

### Example 315

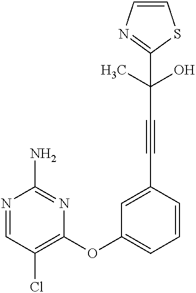

### 4-(3-((2-amino-5-chloro-4-pyrimidinyl)oxy)phenyl)-2-(1,3-thiazol-2-yl)-3-butyn-2-ol (315)

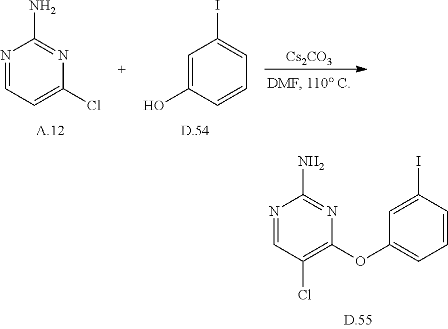

5-Chloro-4-(3-iodophenoxy)pyrimidin-2-amine (D.55): A mixture of A.12 (150 mg, 0.91 mmol), 3-iodophenol D.54 (400 mg, 1.82 mmol), and cesium carbonate (650 mg, 2.0 mmol) in DMF (5 mL) was stirred at 110° C. overnight. After filtration, the filtrate was concentrated and the residue was purified by flash chromatography on silica gel eluting with 2% methanol in dichloromethane to give compound D.55 as a light yellow solid (305 mg, 100%). ¹H NMR (400 MHz, CDCl₃) δ 8.16 (1H, d, J=5.6 Hz), 7.61 (1H, m), 7.53 (1H, s), 7.17-7.12 (2H, m), 6.18 (1H, d, J=5.6 Hz), 5.08 (2H, br s) ppm; LCMS-ESI (POS), M/Z, M+1: Found 348.0, Calculated 348.0.

4-(3-((2-amino-5-chloro-4-pyrimidinyl)oxy)phenyl)-2-(1,3-thiazol-2-yl)-3-butyn-2-ol (315): Using the procedure described in example 303, 52 mg (76%) of 315 was obtained as a light yellow solid from 63 mg (0.2 mmol) of D.55 and 60 mg (0.4 mmol) of 2-(thiazol-2-yl)but-3-yn-2-ol C.4. ¹H NMR (400 MHz, CDCl₃) δ 8.11 (1H, br s), 7.76 (1H, m), 7.30-7.28 (3H, m), 7.17 (1H, s), 7.07 (1H, s), 6.10 (1H, m), 5.41 (2H, br s), 2.05 (3H, s) ppm; LCMS-ESI (POS), M/Z, M+1: Found 373.0, Calculated 373.0.

### Example 316

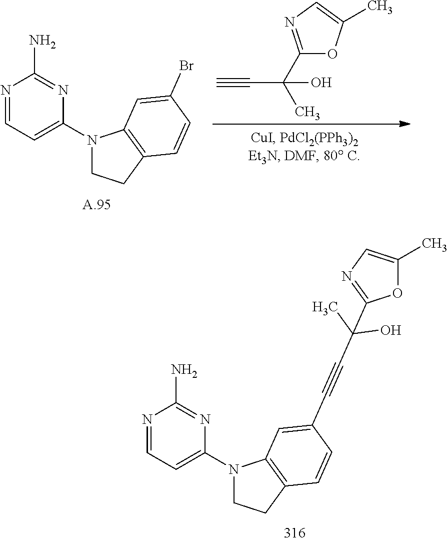

4-(1-(2-Amino-4-pyrimidinyl)-2,3-dihydro-1H-indol-6-yl)-2-(5-methyl-1,3-oxazol-2-yl)-3-Butyn-2-ol (316): Using the procedure described for the preparation of compound 249, 30 mg (17%) of 316 was obtained as a light yellow solid from 146 mg (0.5 mmol) of A.95 and 227 mg (1.5 mmol) of 2-(5-methyloxazol-2-yl)but-3-yn-2-ol C.58. LCMS-ESI (POS), M/Z, M+1: Found 362.2, Calculated 362.2.

### Example 317

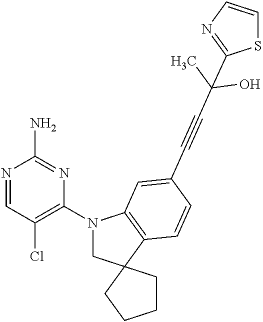

### 4-(1′-(2-amino-5-chloro-4-pyrimidinyl)-1′,2′-dihydrospiro[cyclopentane-1,3′-indol]-6′-yl)-2-(1,3-thiazol-2-yl)-3-butyn-2-ol (317)

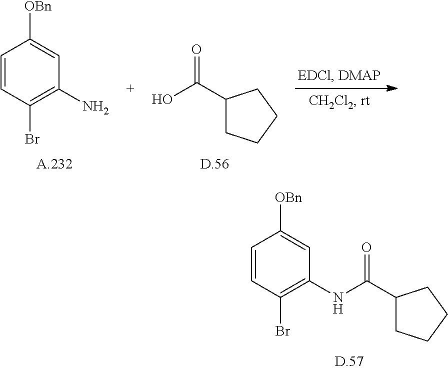

N-(5-(Benzyloxy)-2-bromophenyl)cyclopentanecarboxamide (D.57): To a solution of 5-(benzyloxy)-2-bromoaniline A.232 (6.9 g, 25 mmol) in dichloromethane (50 mL) were added cyclopentanecarboxylic acid D.56 (3.6 g, 25 mmol), EDCI (4.8 g, 25 mmol), DMAP (3.05 g, 25 mmol). The mixture was stirred at room temperature overnight. The reaction mixture was concentrated and the residue was dissolved in ethyl acetate (250 mL) and washed with 1N HCl, brine then dried. The solvent was evaporated and the residue was purified by flash chromatography on silica gel eluting with 10-30% dichloromethane in hexane to give compound D.57 as a white solid (9 g, 96%). LCMS-ESI (POS), M/Z, M+1: Found 374.1, Calculated 374.1.

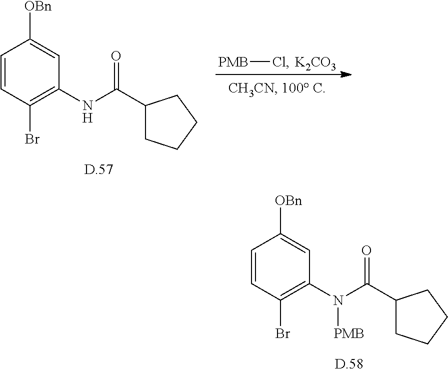

### N-(4-Methoxybenzyl)-N-(5-(benzyloxy)-2-bromophenyl)cyclopentanecarboxamide (D.58)

A mixture of D.57 (8.7 g, 23.26 mmol), 4-methoxybenzyl chloride (7.29 g, 46.52 mmol), and potassium carbonate (9.68 g, 70 mmol) in acetonitrile (250 mL) was stirred at 100° C. for 24 hr. After filtration, the filtrate was concentrated and the residue was purified by chromatography on silica gel eluting with 20-70% ethyl acetate in hexane to give compound D.58 as a white solid (8.21 g, 72%). ¹H NMR (400 MHz, CDCl₃) δ 7.54 (1H, d, J=8.8 Hz), 7.39-7.28 (5H, m), 7.11 (2H, d, J=8.5 Hz), 6.86 (1H, m), 6.81 (2H, d, J=8.5 Hz), 6.31 (1H, d, J=3.0 Hz), 5.58 (1H, d, J=14.2 Hz), 4.87 (1H, d, J=12 Hz), 4.81 (1H, d, J=12 Hz), 3.92 (1H, d, J=14 2 Hz), 3.81 (3H, s), 2.30 (1H, m), 1.96 (1H, m), 1.75-1.55 (4H, m), 1.43-1.39 (3H, m) ppm; LCMS-ESI (POS), M/Z, M+1: Found 494.2, Calculated 494.1.

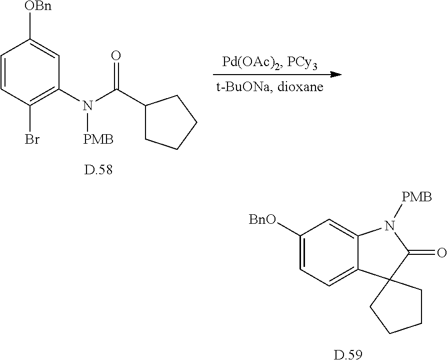

6′-(Benzyloxy)-1′-(4-methoxybenzyl)spiro[cyclopentane-1,3′-indol]-2′(1′H)-one(D.59): A mixture of D.58 (7.0 g, 14.20 mmol), tricyclohexylphosphine (398 mg, 1.42 mmol), palladium (II) acetate (319 mg, 1.42 mmol), t-BuONa (2047 mg, 21.3 mmol) in anhydrous dioxane (70 mL) was stirred at 60° C. overnight. The reaction mixture was poured into iced sat's NH₄Cl then extracted with ethyl acetate (×2). The organic layers were washed with brine and dried. The solvent was evaporated and residue was purified by chromatography on silica gel eluting with 20-50% ethyl acetate in hexane to give compound D.59 as a light yellow oil (4.18 g, 82%). ¹H NMR (400 MHz, CDCl₃) δ 7.41-7.36 (4H, m), 7.20 (2H, m), 7.09 (2H, m), 6.84 (2H, m), 6.58 (1H, m), 6.42 (1H, s), 5.00 (2H, s), 4.80 (2H, s), 3.80 (3H, s), 2.23-2.11 (2H, m), 2.09 (2H, m), 1.99 (2H, m), 1.85 (2H, s) ppm; LCMS-ESI (POS), M/Z, M+1: Found 414.3, Calculated 414.2.

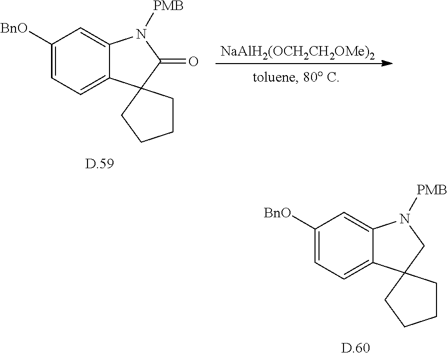

### 6′-(Benzyloxy)-1′-(4-methoxybenzyl)-1′,2′-dihydrospiro[cyclopentane-1,3′-indole] (D.60)

To a solution of D.59 (650 mg, 1.57 mmol) in toluene (15 mL) at 80° C. was added slowly 70% sodium bis(2-methoxyethoxyl)-aluminum hydride solution in toluene (1.37 mL, 4.8 mmol). The reaction mixture was stirred at 80° C. for 1.5 hr. The reaction was quenched with 2N NaOH at 0° C. then poured into iced 2N NaOH and extracted with ethyl acetate. The organic layers were dried and concentrated. The residue was purified by chromatography on silica gel eluting with 0-40% ethyl acetate in hexane to give compound D.60 as a colorless oil (584 mg, 93%). ¹H NMR (400 MHz, CDCl₃) δ 7.45-7.31 (5H, m), 7.28-7.25 (2H, m), 6.95-6.84 (3H, m), 6.32 (1H, d, J=7.4 Hz), 6.23 (1H, br s), 5.02 (2H, s), 4.20 (2H, s), 3.83 (3H, s), 3.14 (2H, s), 1.81-1.66 (8H, m) ppm; LCMS-ESI (POS), M/Z, M+1: Found 400.3, Calculated 400.2.

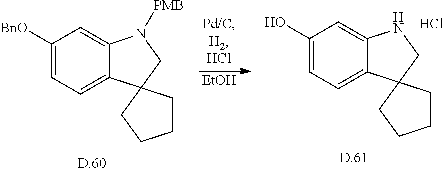

1′,2′-Dihydrospiro[cyclopentane-1,3′-indol]-6′-ol (D.61): A suspension of D.60 (584 mg, 1.46 mmol), 10% palladium on carbon (60 mg) in ethanol (20 mL) containing 2 mL of con. HCl was stirred under H₂ atmosphere for 16 hr. After filtration, the filtrate was concentrated to give compound D.61 as a yellow powder (220 mg, 100%) which was used without further purification. LCMS-ESI (POS), M/Z, M+1: Found 190.2, Calculated 190.1.

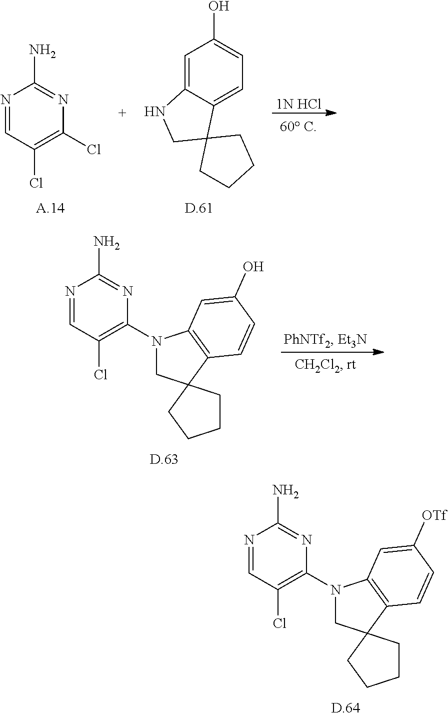

1′-(2-Amino-5-chloro-4-pyrimidinyl)-1′,2′-dihydrospiro[cyclopentane-1,3′-indol]-6′-yl trifluoromethanesulfonate(D.64): A mixture of D.61 (220 mg, 1.2 mmol) and 2-amino-4,5-dichloropyrimidine A.14 (230 mg, 1.4 mmol) in 1N HCl aqueous solution (5 mL) was stirred at 60° C. overnight. The reaction mixture was lyophilized and the solid thus obtained was dissolved in dichloromethane (15 mL). To the solution were added triethylamine (1.13 g, 11.2 mmol, 1558 uL) and N-phenyl-bis(trifluoromethane-sulfonimide (714 mg, 3 mmol). The mixture was stirred at room temperature for 2 hr. The reaction mixture was concentrated and the residue was purified by chromatography on silica gel eluting with 15-50% ethyl acetate in hexane to give compound D.64 as a white solid (295 mg, 45%). ¹H NMR (400 MHz, CDCl₃) δ 8.11 (1H, s), 7.51 (1H, d, J=2.2 Hz), 7.16 (1H, d, J=8.2 Hz), 6.87 (1H, dd, J=8.2 Hz, J=2.2 Hz), 4.97 (2H, br s), 4.17 (2H, s), 1.89-1.79 (8H, m) ppm; LCMS-ESI (POS), M/Z, M+1: Found 449.1, Calculated 449.1.

4-(1′-(2-amino-5-chloro-4-pyrimidinyl)-1′,2′-dihydrospiro[cyclopentane-1,3′-indol]-6′-yl)-2-(1,3-thiazol-2-yl)-3-Butyn-2-ol (317): To a degassed solution of D.64 (88 mg, 0.20 mmol) in piperidine (1 mL) were added tertrakis(triphenylphosphine)palladium (23 mg, 0.02 mmol) and 2-(thiazol-2-yl)but-3-yn-2-ol C.4 (67 mg, 0.40 mmol). The resulting mixture was stirred at 80° C. for 6 hr under argon atmosphere. The reaction mixture was concentrated and residue was purified by chromatography on silica gel eluting with 0.25% NH₄OH and 2.5% methanol in dichloromethane to give compound 317 as a light yellow solid (75 mg, 83%). ¹H NMR (400 MHz, CDCl₃) δ 8.02 (1H, s), 7.78 (1H, d, J=3.2 Hz), 7.59 (1H, s), 7.31 (1H, d, J=3.2 Hz), 7.13 (1H, d, J=7.7 Hz), 7.07 (1H, d, J=7.7 Hz), 5.31 (1H, s), 5.30 (2H, s), 4.06 (2H, s), 2.06 (3H, s), 1.84-1.76 (8H, m) ppm; LCMS-ESI (POS), M/Z, M+1: Found 452.2, Calculated 452.1.

### Example 318

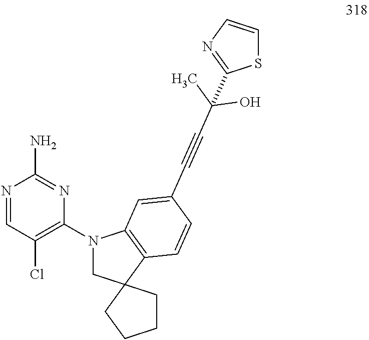

2R)-4-(1′-(2-amino-5-chloro-4-pyrimidinyl)-1′,2′-dihydrospiro[cyclopentane-1,3′-indol]-6′-yl)-2-(1,3-thiazol-2-yl)-3-butyn-2-ol (318): Using the procedure described for the preparation of compound 317, 80 mg (89%) of 318 was obtained as an off-white solid from 88 mg (0.2 mmol) of D.64 and 67 mg (0.4 mmol) of (R)-2-(thiazol-2-yl)but-3-yn-2-ol C.6. ¹H NMR (400 MHz, CDCl₃) δ 8.02 (1H, s), 7.78 (1H, d, J=3.2 Hz), 7.59 (1H, s), 7.31 (1H, d, J=3.2 Hz), 7.13 (1H, d, J=7.7 Hz), 7.07 (1H, d, J=7.7 Hz), 5.20 (2H, br s), 4.07 (2H, s), 2.06 (3H, s), 1.84-1.76 (8H, m) ppm; LCMS-ESI (POS), M/Z, M+1: Found 452.0, Calculated 452.1.

### Example 319

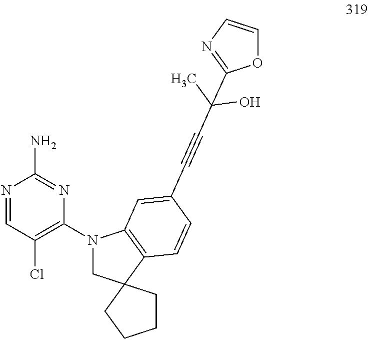

4-(1′-(2-Amino-5-chloro-4-pyrimidinyl)-1′,2′-dihydrospiro[cyclopentane-1,3′-indol]-6′-yl)-2-(1,3-oxazol-2-yl)-3-butyn-2-ol (319): Using the procedure described for the preparation of compound 317, 60 mg (69%) of 319 was obtained as a light yellow solid from 88 mg (0.2 mmol) of D.64 and 55 mg (0.4 mmol) of 2-(oxazol-2-yl)but-3-yn-2-ol C.57 . ¹H NMR (400 MHz, CDCl₃) δ 8.01 (1H, s), 7.64 (1H, s), 7.58 (1H, s), 7.12 (1H, s), 7.09 (1H, d, J=8 Hz), 7.06 (1H, d, J=8 Hz), 5.42 (2H, br s), 5.30 (1H, s), 4.05 (2H, s), 2.02 (3H, s), 1.82-1.74 (8H, m) ppm; δ ppm; LCMS-ESI (POS), M/Z, M+1: Found 436.2, Calculated 436.2.

### Biological Testing

### NIK Assays

### Biochemical (Enzyme) Assays

Two formats of biochemical assays are implemented: (1) chemiluminescent (CL) assay for NIK IC₅₀ determination; (2) homogenous time resolved fluorescence (HTRF) assay for NIK Kᵢ measurement. Both assays utilize a truncated form of NIK containing only the catalytic domain with substantially increased autophosphorylation activity compared to the full-length protein. The NIK proteins are purified with baculovirus expression system and further biotinylated with N-hydroxysuccinamide biotin ester (Sigma). For the CL assay, 50 nM of biotinylated-NIK are incubated with compounds and 50 μM of ATP in a buffer containing 20 mM of Tris-HCl pH7.5, 40 mM of MgCl₂, and 1.5 mM of DTT at room temperature for 1 hour. The reaction mixtures are then immobilized on a Streptavidin-coated plate (Pierce). The autophosphorylation activity is detected with an anti-phopho-serine/threonine antibody (Upstate) and a horseradish peroxidase (HRP)-conjugated secondary antibody (Promega). For the HTRF assay, 10 nM of enzyme are incubated with compounds in the presence of 20, 100, 200, or 500 μM of ATP in a buffer with 20 mM of Hepes pH7.5, 20 mM of MgCl₂, 0.2% of Tween 20 and 1 mM of DTT at room temperature for 2 hours. The reactions are stopped by adding 5 mM of EDTA, 12.5 ng/ml of Eu³′-labeled anti-phopho-serine/threonine antibody and 1 nM of Streptavidin-Alexa647 (Invitrogen). After 2 hour incubation, the fluorescence is measure at 615 nm and 665 nm, while the specific enzymatic activity is calculated by the signal ratio between 665 nm and 615 nm.

### Biological Assay Results

Examples of compounds of the invention that have an Kᵢ activity lower than 1 μM in the homogenous time resolved fluorescence (HTRF) assay for NIK are Example 1-5, 7, 8, 12-16, 18-23, 25, 26, 29-34, 36-40, 44-50, 52-80, 82-91, 93-114, 116-122, 124-137, 139, 143-146, 148-152, 154-167, 171, 173, 176, 179-181, 182-188, 190-193, 195, 197, 198, 200, 202-208, 209, 210, 211-225, 227-229, 231-234, 237-241, 243-250, 256, 259, 262, 264, 265, 267, 269-271, 273-278, 280-282, 284, 288-312, and 314-319. Examples ranging from 1 μM to 30 μM are Example 6, 9-11, 17, 24, 28, 35, 41-43, 81, 92, 147, 153, 168-170, 172, 174, 175, 177, 178, 182a, 194, 196, 199, 201, 209a, 211a, 226, 230, 236, 242, 251-255, 257, 258, 260, 261, 266, 268, 272, 279, 283, 285, 287, and 313.

Examples of compounds of the invention that have an IC₅₀ activity lower than 1 μM in the chemiluminescent (CL) assay for NIK are Example 51, 115, 123, 138, 140, 189, 235, 263, and 286.

### Cell-Based Assay (HT29 IC₅₀)

To determine cellular potency of NIK inhibitors, an enzyme-linked DNA protein interaction assay (ELDIA) that quantitatively measures the p52 level in the cells is established. HT-29 cells are incubated with different concentrations of compounds for 1 hour and further stimulated with 100 ng/ml of LTα/β2 (R&D System) for 4-6 hours. Equal amounts of whole cell lysates are then incubated with the double stranded DNA oligonucleotide containing p52 binding site immobilized on a Streptavidin-coated plate in a binding buffer consisting of 5 mM of HEPES pH7.5, 120 mM of KCl, 8% of glycerol, 1% of BSA, 2 mM of DTT, and 10 μg/ml of Herring sperm DNA. After 1 hour incubation, the DNA-bound p52 proteins will be detected by an anti-p52 monoclonal antibody (Upstate) and a HRP-linked secondary antibody.

### Formulations

Also embraced within this invention is a class of pharmaceutical compositions comprising the active compounds of the current invention in association with one or more non-toxic, pharmaceutically-acceptable carriers and/or diluents and/or adjuvants (collectively referred to herein as “carrier” materials) and, if desired, other active ingredients. The active compounds of the present invention may be administered by any suitable route, preferably in the form of a pharmaceutical composition adapted to such a route, and in a dose effective for the treatment intended. The compounds and compositions of the present invention may, for example, be administered orally, mucosally, topically, rectally, pulmonarily such as by inhalation spray, or parentally including intravascularly, intravenously, intraperitoneally, subcutaneously, intramuscularly intrasternally and infusion techniques, in dosage unit formulations containing conventional pharmaceutically acceptable carriers, adjuvants, and vehicles.

The pharmaceutically active compounds of this invention can be processed in accordance with conventional methods of pharmacy to produce medicinal agents for administration to patients, including humans and other mammals.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are tablets or capsules. For example, these may contain an amount of active ingredient from about 1 to 2000 mg, preferably from about 1 to 500 mg. A suitable daily dose for a human or other mammal may vary widely depending on the condition of the patient and other factors, but, once again, can be determined using routine methods.

The amount of compounds which are administered and the dosage regimen for treating a disease condition with the compounds and/or compositions of this invention depends on a variety of factors, including the age, weight, sex and medical condition of the subject, the type of disease, the severity of the disease, the route and frequency of administration, and the particular compound employed. Thus, the dosage regimen may vary widely, but can be determined routinely using standard methods. A daily dose of about 0.01 to 500 mg/kg, preferably between about 0.01 and about 50 mg/kg, and more preferably about 0.01 and about 30 mg/kg body weight may be appropriate. The daily dose can be administered in one to four doses per day.

For therapeutic purposes, the active compounds of this invention are ordinarily combined with one or more adjuvants appropriate to the indicated route of administration. If administered per os, the compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

In the case of psoriasis and other skin conditions, it may be preferable to apply a topical preparation of compounds of this invention to the affected area two to four times a day.

Formulations suitable for topical administration include liquid or semi-liquid preparations suitable for penetration through the skin (e.g., liniments, lotions, ointments, creams, or pastes) and drops suitable for administration to the eye, ear, or nose. A suitable topical dose of active ingredient of a compound of the invention is 0.1 mg to 150 mg administered one to four, preferably one or two times daily. For topical administration, the active ingredient may comprise from 0.001% to 10% w/w, e.g., from 1% to 2% by weight of the formulation, although it may comprise as much as 10% w/w, but preferably not more than 5% w/w, and more preferably from 0.1% to 1% of the formulation.

When formulated in an ointment, the active ingredients may be employed with either paraffinic or a water-miscible ointment base. Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound, which enhances absorption or penetration of the active ingredient through the skin or other affected areas. Examples of such dermal penetration enhancers include DMSO and related analogs.

The compounds of this invention can also be administered by a transdermal device. Preferably transdermal administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier, which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base, which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, sodium lauryl sulfate, glyceryl distearate alone or with a wax, or other materials well known in the art.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredients are dissolved or suspended in suitable carrier, especially an aqueous solvent for the active ingredients. The active ingredients are preferably present in such formulations in a concentration of 0.5 to 20%, advantageously 0.5 to 10% and particularly about 1.5% w/w.

Formulations for parenteral administration may be in the form of aqueous or non-aqueous isotonic sterile injection solutions or suspensions. These solutions and suspensions may be prepared from sterile powders or granules using one or more of the carriers or diluents mentioned for use in the formulations for oral administration or by using other suitable dispersing or wetting agents and suspending agents. The compounds may be dissolved in water, polyethylene glycol, propylene glycol, ethanol, corn oil, cottonseed oil, peanut oil, sesame oil, benzyl alcohol, sodium chloride, tragacanth gum, and/or various buffers. Other adjuvants and modes of administration are well and widely known in the pharmaceutical art. The active ingredient may also be administered by injection as a composition with suitable carriers including saline, dextrose, or water, or with cyclodextrin (ie. Captisol), cosolvent solubilization (ie. propylene glycol) or micellar solubilization (ie. Tween 80).

The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed, including synthetic mono- or diglycerides. In addition, fatty acids such as oleic acid find use in the preparation of injectables.

For pulmonary administration, the pharmaceutical composition may be administered in the form of an aerosol or with an inhaler including dry powder aerosol.

Suppositories for rectal administration of the drug can be prepared by mixing the drug with a suitable non-irritating excipient such as cocoa butter and polyethylene glycols that are solid at ordinary temperatures but liquid at the rectal temperature and will therefore melt in the rectum and release the drug.

The pharmaceutical compositions may be subjected to conventional pharmaceutical operations such as sterilization and/or may contain conventional adjuvants, such as preservatives, stabilizers, wetting agents, emulsifiers, buffers etc. Tablets and pills can additionally be prepared with enteric coatings. Such compositions may also comprise adjuvants, such as wetting, sweetening, flavoring, and perfuming agents.

The foregoing is merely illustrative of the invention and is not intended to limit the invention to the disclosed compounds. Variations and changes, which are obvious to one skilled in the art are intended to be within the scope and nature of the invention, which are defined, in the appended claims.

From the foregoing description, one skilled in the art can easily ascertain the essential characteristics of this invention, and without departing from the spirit and scope thereof, can make various changes and modifications of the invention to adapt it to various usages and conditions.

No unacceptable toxological effects are expected when compounds of the present invention are administered in accordance with the present invention.

All mentioned references, patents, applications and publications, are hereby incorporated by reference in their entirety, as if here written.

1. A compound of formula I 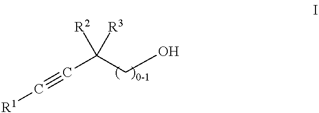
enantiomers, diastereomers and salts thereof wherein
R¹ is selected from 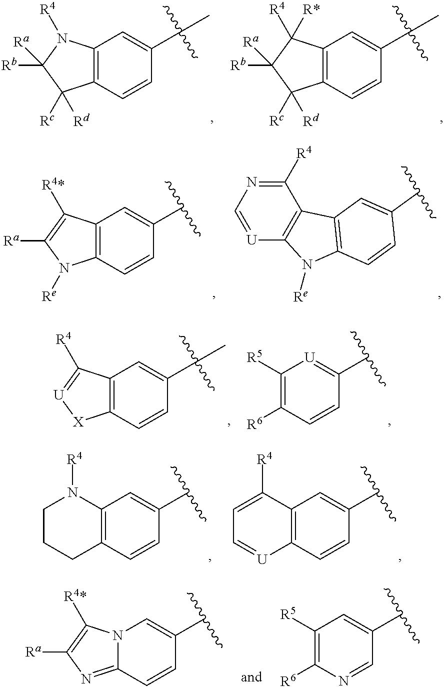
any of which may be optionally substituted with one or more R^{x} groups as allowed by valance;
provided that when R¹ is 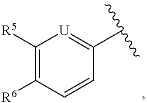
where U is CH and R⁶ is H, then R⁵
is other than 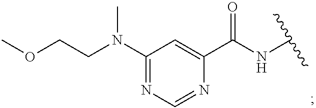
R² is alkyl or haloalkyl;
R³ is alkyl, cycloalkyl, haloalkyl, —C(═O)R⁷, —C(═O)OR⁷, —C(═O)NR⁸R⁹, aryl or heteroaryl wherein either of said aryl or heteroaryl may be optionally substituted with one or more R^{x} as allowed by valance;
or R² and R³ together with the carbon atom to which they are attached may combine to form 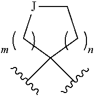
which may be optionally substituted with one or more R^{x} groups as allowed by valance;
R⁴ and R⁴* are independently
i) pyridyl, pyrimidyl, pyrazinyl, triazinyl, purinyl, pyrrolopyrimidyl, triazolopyrimidyl, furopyrimidyl, thienopyrimidyl, oxazolopyrimidyl, or thiazolopyrimidyl, each of which is substituted with at least one R¹⁰ group, and any of which may be optionally substituted with one or more R^{x} groups as allowed by valance; or
ii) oxazolyl, isoxazolyl, oxadiazolyl, thiazolyl, isothiazolyl, thiadiazolyl, or —C(═O)R⁷* any of which may be optionally substituted with one or more R^{x} groups as allowed by valance;
provided R⁴ is other than —C(═O)R⁷* when R¹ is 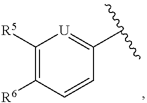
where U is CH and R⁶ is H, and R⁵ is either —(CH₂)ₖ—N(R⁸)(R⁴) or —(CH₂)ₖ—R⁴;
R⁵ is —(CH₂)ₖ—R⁴, —(CH₂)ₖ—N(R⁸)(R⁴), —(CH₂)ₖ—OR⁴, —(CH₂)ₖ—C(═O)R⁴; —(CH₂)ₖ—C(═O)OR⁴, —(CH₂)ₖ—C(═O)N(R⁸)(R⁴) or —(CH₂)ₖ—NR⁸—(C═O)R⁴;
R⁶ is H, halo, alkyl, —(CH₂)ₖ—OR¹¹, —(CH₂)ₖ—N(R¹²)(R¹³), —(CH₂)ₖ—C(═O)R¹¹, —(CH₂)ₖ—C(═O)OR¹¹;
R⁷, R⁷* and R⁷⁺are each independently
(i) H, or
(ii) alkyl, alkenyl, alkynyl, cycloalkyl, heterocyclyl, aryl, heteroaryl, cycloalkylalkyl, heterocyclylalkyl, arylalkyl, heteroarylalkyl, alkoxyalkyl, hydroxyalkyl or NR⁸R⁹-alkyl any of which may be optionally substituted with one or more R^{x} groups as allowed by valance;
R⁸, R⁹, R⁸⁺and R⁹⁺ are each independently
(i) H;
(ii) alkyl, alkenyl, alkynyl, cycloalkyl, heterocyclyl, aryl, heteroaryl, cycloalkylalkyl, heterocyclylalkyl, arylalkyl, heteroarylalky, alkoxyalkyl, hydroxyalkyl or (NR¹²R¹³)-alkyl, any of which may be optionally substituted with one or more R^{x} groups as allowed by valance;
(iii) or R⁸ and R⁹ together with the nitrogen atom to which they are attached may combine to form a heterocyclyl ring optionally substituted with one or more R^{x} groups as allowed by valance;
(iv) or R⁸⁺ and R⁹⁺ together with the nitrogen atom to which they are attached may combine to form a heterocyclyl ring optionally substituted with one or more R^{x} groups as allowed by valance;
R¹⁰ is H, —NR¹⁴R¹⁵, or —C(═O)NR¹⁴R¹⁵;
R¹¹ is
(i) H, or
(ii) alkyl, alkenyl, alkynyl, cycloalkyl, heterocyclyl, aryl, heteroaryl, cycloalkylalkyl, heterocyclylalkyl, arylalkyl or heteroarylalkyl any of which may be optionally substituted with one or more R^{x} groups as allowed by valance;
R¹² and R¹³ are each independently
(i) H;
(ii) alkyl, alkenyl, alkynyl, cycloalkyl, heterocyclyl, aryl, heteroaryl, cycloalkylalkyl, heterocyclylalkyl, arylalkyl, heteroarylalkyl, alkoxyalkyl, hydroxyalkyl or (NR⁸R⁹)-alkyl, any of which may be optionally substituted with one or more R^{x} groups as allowed by valance;
(iii) or R¹² and R¹³ together with the nitrogen atom to which they are attached may combine to form a heterocyclyl ring optionally substituted with one or more R^{x} groups as allowed by valance;
R¹⁴ and R¹⁵ are each independently
(i) H;
(ii) alkyl, alkenyl, alkynyl, cycloalkyl, heterocyclyl, aryl, heteroaryl, cycloalkylalkyl, heterocyclylalkyl, arylalkyl, heteroarylalkyl, alkoxyalkyl, hydroxyalkyl or (NR¹²R¹³)-alkyl, any of which may be optionally substituted with one or more R^{x} groups as allowed by valance;
(iii) or R¹⁴ and R¹⁵ together with the nitrogen atom to which they are attached may combine to form a heterocyclyl ring optionally substituted with one or more R^{x} groups as allowed by valance;
R^{a}, R^{b}, R^{c} R^{d} and R* are each independently H or R^{x}
or R^{a} and R^{b} together with the carbon atom to which they are attached may combine to form a carbonyl group;
or R^{b} together with either R^{c} or R* may combine to form a bond;
or R^{a} and R^{e}, together with the atoms to which they are respectively attached, may combine to form 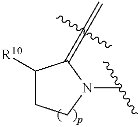
which may be optionally substituted with one or more R^{x} groups as allowed by valance;
or R^{a} and R⁴*, together with the atoms to which they are respectively attached, may combine to form 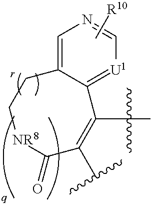
which may be optionally substituted with one or more R^{x} groups as allowed by valance;
or R^{c} and R^{d} together with the carbon atom to which they are each attached may combine to form 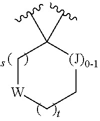
which may be optionally substituted with one or more R^{x} groups as allowed by valance;
R^{e} is H, R⁴, or R^{x};
R^{x} is an optional substituent independently selected at each occurrence from halo, cyano, nitro, oxo, alkyl, haloalkyl, alkenyl, alkynyl, cycloalkyl, cycloalkenyl, heterocyclo, aryl, heteroaryl, arylalkyl, heteroarylalkyl, cycloalkylalkyl, heterocycloalkyl, -(alkylene)_{q}-OR⁷⁺, -(alkylene)_{q}-S(O)ₙR⁷⁺, -(alkylene)_{q}-NR⁸⁺R⁹⁺, -(alkylene)_{q}-C(═O)R⁷⁺, -(alkylene)_{q}-C(═S)R⁷⁺, -(alkylene)_{q}-C(═O)OR⁷⁺, -(alkylene)_{q}-OC(═O)R⁷⁺, -(alkylene)_{q}-C(═S)OR⁷⁺, -(alkylene)_{q}-C(═O)NR⁸⁺R⁹⁺, -(alkylene)_{q}-C(═S)NR⁸⁺R⁹⁺, -(alkylene)_{q}-N(R¹⁵)C(═O)NR⁸⁺R⁹⁺, -(alkylene)_{q}-N(R¹⁵)C(═S)NR⁸⁺R⁹⁺, -(alkylene)_{q}-N(R¹⁵)C(═O)R⁷⁺, -(alkylene)_{q}-N(R¹⁵)C(═S)R⁷⁺, -(alkylene)_{q}-OC(═O)NR⁸⁺R⁹⁺, -(alkylene)_{q}-OC(═S)NR⁸⁺R⁹⁺, -(alkylene)_{q}-SO₂NR⁸⁺R⁹⁺, -(alkylene)_{q}-N(R¹⁵)SO₂e, -(alkylene)_{q}-N(R¹⁵)SO₂NR⁸⁺R⁹⁺, -(alkylene)_{q}-N(R¹⁵)C(═O)OR⁷⁺, -(alkylene)_{q}-N(R¹⁵)C(═S)OR⁷⁺, or -(alkylene)_{q}-N(R¹⁵)SO₂R⁷⁺;
wherein alkylene, alkyl, haloalkyl, alkenyl, alkynyl, cycloalkyl, cycloalkenyl, heterocyclo, aryl, heteroaryl, arylalkyl, heteroarylalkyl, cycloalkylalkyl, and heterocycloalkyl groups may be further independently substituted with one or more -(alkylene)_{q}-CN, -(alkylene)_{q}-OR⁷⁺, -(alkylene)_{q}-S(O)ₙR⁷⁺, -(alkylene)_{q}-NR⁸⁺R⁹⁺, -(alkylene)_{q}-C(═O)R⁷⁺, -(alkylene)_{q}-C(═S)R⁷⁺, -(alkylene)_{q}-C(═O)OR⁷⁺, -(alkylene)_{q}-OC(═O)R⁷⁺, -(alkylene)_{q}-C(═S)OR⁷⁺, -(alkylene)_{q}-C(═O)NR⁸⁺R⁹⁺, -(alkylene)_{q}-C(═S)NR⁸⁺R⁹⁺, -(alkylene)_{q}-N(R¹⁵)C(═O)NR⁸⁺R⁹⁺, -(alkylene)_{q}-N(R¹⁵)C(═S)NR⁸⁺R⁹⁺, -(alkylene)_{q}-N(R¹⁵)C(═O)R⁷⁺, -(alkylene)_{q}-N(R¹⁵)C(═S)R⁷⁺, -(alkylene)_{q}-OC(═O)NR⁸⁺R⁹⁺, -(alkylene)_{q}-OC(═S)NR⁸⁺R⁹⁺, -(alkylene)_{q}-SO₂NR⁸⁺R⁹⁺, -(alkylene)_{q}-N(R¹⁵)SO₂R⁷⁺, -(alkylene)_{q}-N(R¹⁵)SO₂NR⁸⁺R⁹⁺, -(alkylene)_{q}-N(R¹⁵)C(═O)OR⁷⁺, -(alkylene)_{q}-N(R¹⁵)C(═S)OR⁷⁺, or -(alkylene)_{q}-N(R¹⁵)SO₂R⁷⁺;
J and W are independently —CH₂—, —N(R⁸)—, —O—, or —S(═O)ᵥ—,
X is —O— or —S(═O)ᵥ—
U and U¹ are independently CH or N
k at each occurrence is independently 0, 1, 2 or 3;
m is 1, 2 or 3
n is 0, 1 or 2
p is 1, 2 or 3
q at each occurrence is independently 0 or 1
r is 0, 1, 2 or 3;
s is 1, 2 or 3
t is 0, 1 or 2
v at each occurrence is independently 0, 1 or 2

2. A compound of claim 1 wherein R¹ is selected from

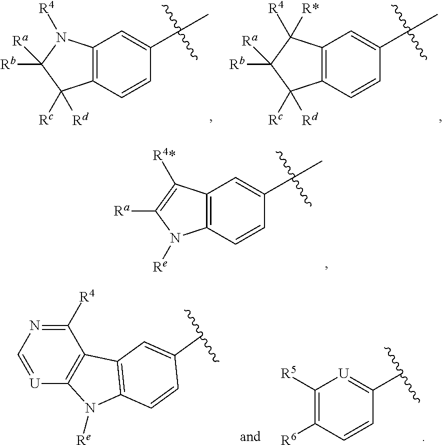

3. A compound of claim 1 wherein
R² is alkyl; and
R³ is pyrrolyl, furanyl, thienyl, imidazolyl, pyrazolyl, oxazolyl, isoxazolyl, thiazolyl, isothiazolyl, phenyl, pyridyl, pyrimidyl, pyrazinyl, or oxadiazolyl any of which may be optionally substituted with one or more R^{x} as allowed by valance.
or R² and R³ together with the carbon atom to which they are attached may combine to form 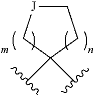
which may be optionally substituted with one or more R^{x} groups as allowed by valance;

4. A compound of claim 2 wherein
R² is alkyl; and
R³ is pyrrolyl, furanyl, thienyl, imidazolyl, pyrazolyl, oxazolyl, isoxazolyl, thiazolyl, isothiazolyl, phenyl, pyridyl, pyrimidyl, pyrazinyl, or oxadiazolyl any of which may be optionally substituted with one or more R^{x} as allowed by valance;
or R² and R³ together with the carbon atom to which they are attached may combine to form 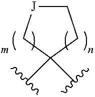
which may be optionally substituted with one or more R^{x} groups as allowed by valance;

5. A compound of claim 3 wherein R², R³ and the carbon atom to which they are attached are selected from 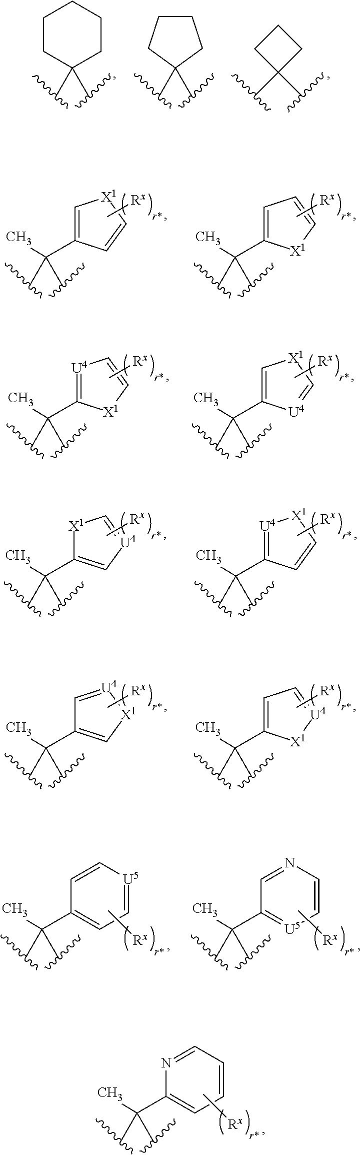
wherein
U⁴ and U⁵ are each independently N or CH
X¹ is NH, O or S(O)ᵥ
r* is 0 or an integer up to three as allowed by valance.

6. A compound of claim 4 wherein R², R³ and the carbon atom to which they are attached are selected from 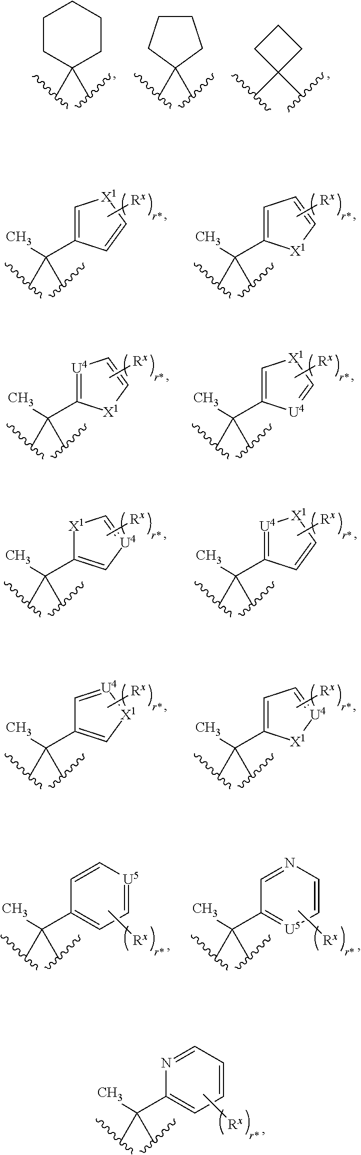
wherein
U⁴ and U⁵ are each independently N or CH
X¹ is NH, O or S(O)ᵥ
r* is 0 or an integer up to three as allowed by valance.

7. A compound according to claim 1, wherein R⁴ is 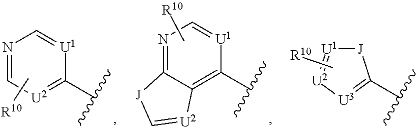
or —C(═O)R⁷*,
any of which may be optionally substituted, as allowed by valance, with up to three R^{x} groups independently selected from halo, cyano, alkyl, haloalkyl, alkenyl, alkynyl, aryl, -(alkylene)_{q}-NR⁸⁺R⁹⁺, -(alkylene)_{q}-C(═O)NeR⁹⁺, -(alkylene)_{q}-OR⁷⁺,
wherein R¹⁰ is H, —NR¹⁴R¹⁵, or —C(═O)NR¹⁴R¹⁵; and
U¹, U² and U³ are independently CH or N.

8. A compound of claim 7 wherein R⁴ is

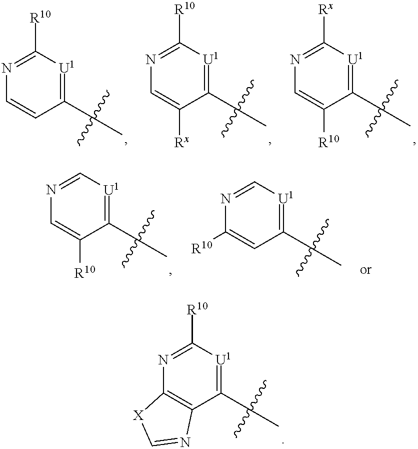

9. A compound of claim 1 having the following formula IA

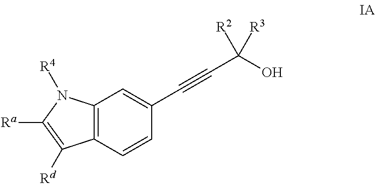

10. A compound of claim 9 wherein
R² is alkyl; and
R³ is pyrrolyl, furanyl, thienyl, imidazolyl, pyrazolyl, oxazolyl, isoxazolyl, thiazolyl, isothiazolyl, phenyl, pyridyl, pyrimidyl, pyrazinyl, or oxadiazolyl any of which may be optionally substituted with one or more R^{x} as allowed by valance;
or R² and R³ together with the carbon atom to which they are attached may combine to form 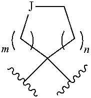
which may be optionally substituted with one or more R^{x} groups as allowed by valance;

11. A compound of claim 10 wherein R², R³ and the carbon atom to which they are attached are selected from 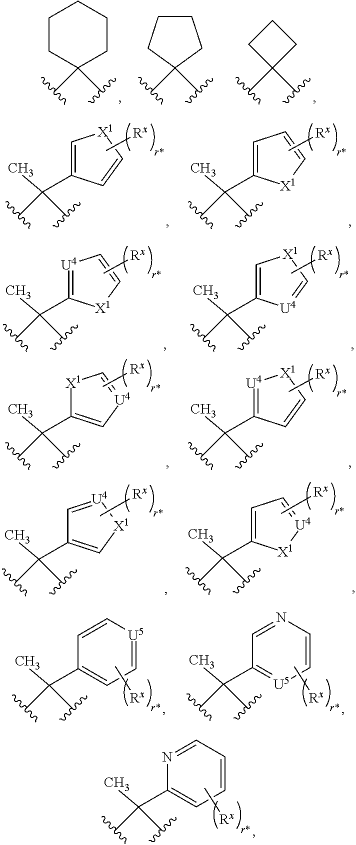
wherein
U⁴ and U⁵ are each independently N or CH
X¹ is NH, O or S(O)ᵥ
r* is 0 or an integer up to three as allowed by valance.

12. A compound according to claim 9, wherein R⁴ is 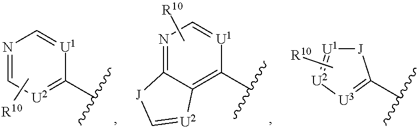
or —C(═O)R⁷*,
any of which may be optionally substituted, as allowed by valance, with up to three R^{x} groups independently selected from halo, cyano, alkyl, haloalkyl, alkenyl, alkynyl, aryl, -(alkylene)_{q}-NR⁸⁺R⁹⁺, -(alkylene)_{q}-C(═O)NR⁸⁺R⁹⁺, -(alkylene)_{q}-OR⁷⁺,
wherein R¹⁰ is H, —NR¹⁴R¹⁵, or —C(═O)NR¹⁴R¹⁵; and
U¹, U² and U³ are independently CH or N.

13. A compound of claim 12 wherein R⁴ is

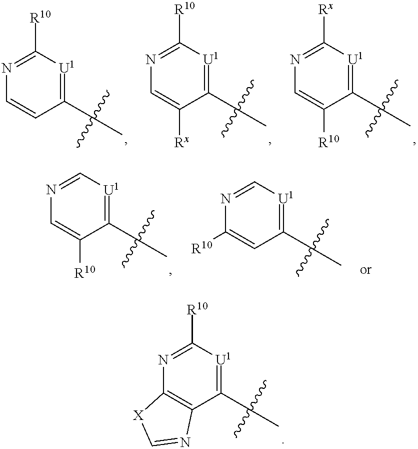

14. A compound of claim 1 having the following formula IB 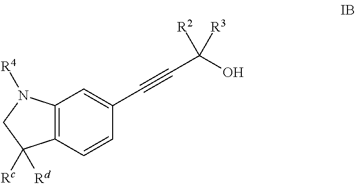
wherein
R^{c} and R^{d} are independently hydrogen, alkyl, (hydroxy)alkyl, (alkoxy)alkyl and ((alkoxy)alkoxy)alkyl;
or R^{e} and R^{d} together with the carbon atom to which they are each attached may combine to form 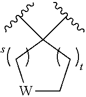
which may be optionally substituted with one
or more R^{x} groups as allowed by valance;

15. A compound of claim 14 wherein
R² is alkyl; and
R³ is pyrrolyl, (uranyl, thienyl, imidazolyl, pyrazolyl, oxazolyl, isoxazolyl, thiazolyl, isothiazolyl, phenyl, pyridyl, pyrimidyl, pyrazinyl, or oxadiazolyl any of which may be optionally substituted with one or more R^{x} as allowed by valance;
or R² and R³ together with the carbon atom to which they are attached may combine to form 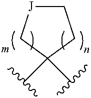
which may be optionally substituted with one or more R^{x} groups as allowed by valance;

16. A compound of claim 15 wherein R², R³ and the carbon atom to which they are attached are selected from 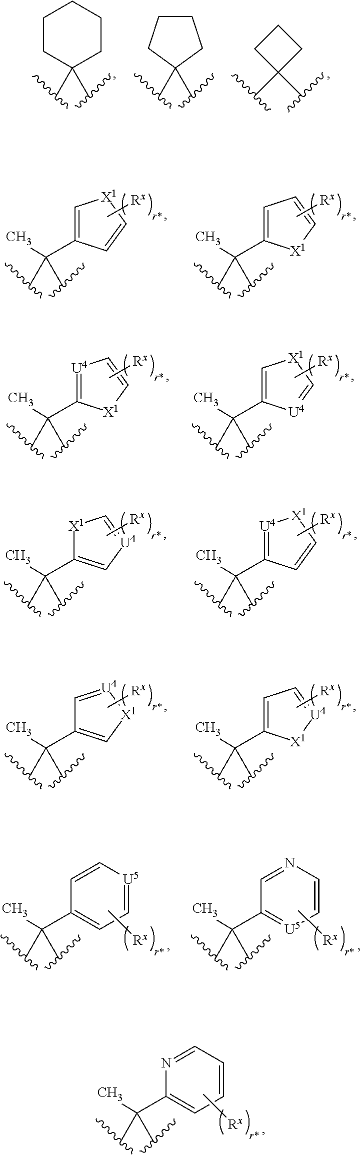
wherein
U⁴ and U⁵ are each independently N or CH
X¹ is NH, O or S(O)ᵥ
r* is 0 or an integer up to three as allowed by valance.

17. A compound according to claim 14, wherein R⁴ is 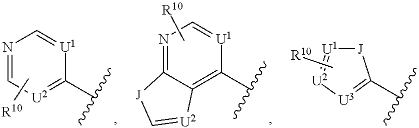
or —C(═O)R⁷*,
any of which may be optionally substituted, as allowed by valance, with up to three R^{x} groups independently selected from halo, cyano, alkyl, haloalkyl, alkenyl, alkynyl, aryl, -(alkylene)_{q}-NR⁸⁺R⁹⁺, -(alkylene)_{q}-C(═O)NR⁸⁺R⁹⁺, -(alkylene)_{q}-OR⁷⁺,
wherein R¹⁰ is H, —NR¹⁴R¹⁵, or —C(═O)NR¹⁴R¹⁵; and
U¹, U² and U³ are independently CH or N.

18. A compound of claim 17 wherein R⁴ is

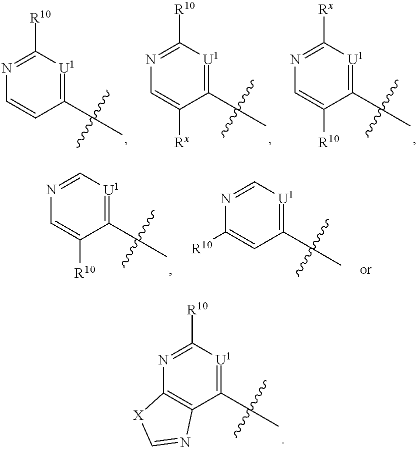

19. A compound of claim 1 having the following formula IC

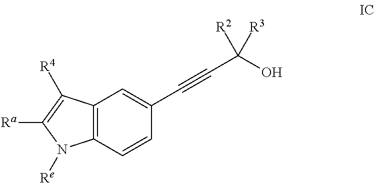

20. A compound of claim 19 wherein
R² is alkyl; and
R³ is pyrrolyl, furanyl, thienyl, imidazolyl, pyrazolyl, oxazolyl, isoxazolyl, thiazolyl, isothiazolyl, phenyl, pyridyl, pyrimidyl, pyrazinyl, or oxadiazolyl any of which may be optionally substituted with one or more R^{x} as allowed by valance;
or R² and R³ together with the carbon atom to which they are attached may combine to form 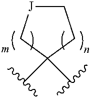
which may be optionally substituted with one or more R^{x} groups as allowed by valance;

21. A compound of claim 20 wherein R², R³ and the carbon atom to which they are attached are selected from 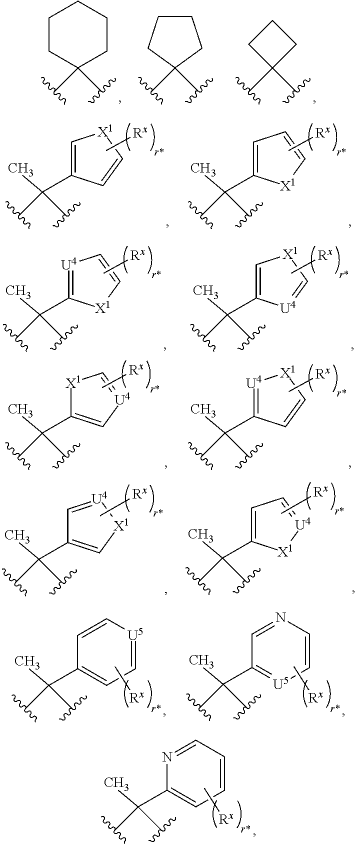
wherein
U⁴ and U⁵ are each independently N or CH
X¹ is NH, O or S(O)ᵥ
r* is 0 or an integer up to three as allowed by valance.

22. A compound according to claim 19, wherein R⁴ is 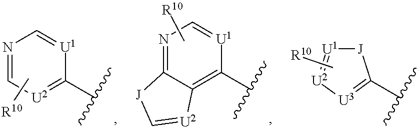
or —C(═O)R⁷*,
any of which may be optionally substituted, as allowed by valance, with up to three R^{x} groups independently selected from halo, cyano, alkyl, haloalkyl, alkenyl, alkynyl, aryl, -(alkylene)_{g}—NR⁸⁺R⁹⁺, -(alkylene)_{q}-C(═O)NR⁸⁺R⁹⁺, -(alkylene)_{q}-OR⁷⁺,
wherein R¹⁰ is H, —NR¹⁴R¹⁵, or —C(═O)NR¹⁴R¹⁵; and
U¹, U² and U³ are independently CH or N.

23. A comnound of claim 22 wherein R⁴ is

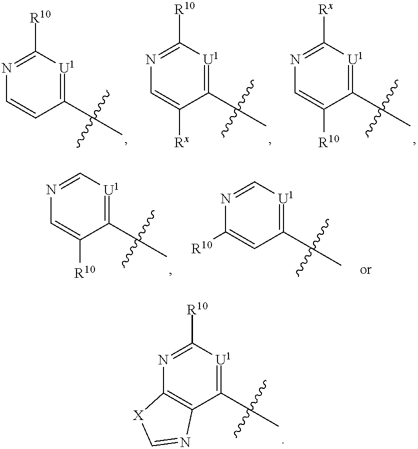

24. A pharmaceutical composition comprising a compound according to claim 1 together with a pharmaceutically acceptable vehicle, adjuvant or diluent.

25. A method of treating an inflammatory condition comprising administering to a patient in need of such treatment a pharmaceutically effective amount of a compound according to claim 1.

26. A method of claim 25 wherein the inflammatory condition is selected from asthma, COPD, rhinitis, multiple sclerosis, IBD, arthritis, rheumatoid arthritis, dermatitis endometriosis and transplant rejection.

27. A method of treating lymphoma, leukemia or multiple myeloma comprising administering to a patient in need of such treatment a pharmaceutically effective amount of a compound according to claim 1.
